(19) United States
(12) Patent Application Publication
(10) Pub. No.: US 2011/0229451 A2
(43) Pub. Date: Sep. 22, 2011

(54) TEMPERATURE SENSITIVE MUTANTS OF MATRIX METALLOPROTEASES AND USES THEREOF

(75) Inventors: Louis Bookbinder, San Diego, CA (US); Gregory Frost, Del Mar, CA (US); Gilbert Keller, Belmont, CA (US); Gerhard Frey, San Diego, CA (US); Hwai Chang, San Marcos, CA (US); Jay Short, Del Mar, CA (US)

(73) Assignee: HALOZYME, INC., San Diego, CA (US)

(21) Appl. No.: 12/660,894

(22) Filed: Mar. 5, 2010

Related U.S. Application Data
(60) Provisional application No. 61/209,366, filed on Mar. 6, 2009.

(51) Int. Cl. A61K 38/48; A61K 38/47; A61P 17/02; A61P 19/00; A61P 17/00; C12N 9/50; C12N 9/96; C07H 21/04; C12N 15/63; C12N 5/10

(52) U.S. Cl. 424/94.62; 424/94.67; 435/219; 435/188; 536/23.2; 435/320.1; 435/325

(57) ABSTRACT

Provided are modified matrix metalloprotease (MMP) enzymes that exhibit temperature-dependent activity and uses thereof. The MMPs can be used, for example, to treat ECM-mediated diseases or disorders characterized by increased deposition or accumulation of one or more ECM components.

### RELATED APPLICATIONS

Benefit of priority is claimed to U.S. Provisional Application Ser. No. 61/209,366, to Louis Bookbinder, Gregory I. Frost, Gilbert Keller, Gerhard Johann Frey, Hwai Wen Chang and Jay Milton Short, entitled “Temperature Sensitive Mutants of Matrix Metalloproteases and Uses Thereof,” filed Mar. 6, 2009. The subject matter of the above-noted application is incorporated by reference in its entirety.

This application is related to International PCT Application Serial No. PCT/US2010/026444, entitled “Temperature Sensitive Mutants of Matrix Metalloproteases and Uses Thereof,” which claims priority to U.S. Provisional Application Ser. No. 61/209,366. This application is related to International PCT Application Serial No. PCT/US2009/001486 to Gilbert Keller and Gregory Frost, and to U.S. application Ser. No. 12/381,063 to Gilbert Keller and Gregory Frost, each entitled “In Vivo Temporal Control of Activatable Matrix-Degrading Enzymes,” and each which claim priority to U.S. Provisional Application Ser. No. 61/068,667 and to U.S. Provisional Application Ser. No. 61/127,725.

The subject matter of the above-noted related applications is incorporated by reference in its entirety.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy # 1 Replacement 04/28/10 and Copy # 2 Replacement 04/28/10), the contents of which are incorporated by reference in their entirety. The computer-readable file on each of the aforementioned compact discs, created on Apr. 28, 2010, is identical, 12.8 megabytes in size, and titled 3077SEQ.002.txt.

### FIELD OF THE INVENTION

Provided are modified matrix metalloprotease (MMP) enzymes that exhibit temperature-dependent activity and uses thereof. MMPs having a controlled duration of action can be used, for example, to treat ECM-mediated diseases or disorders characterized by increased deposition or accumulation of one or more ECM components.

### BACKGROUND

The extracellular matrix (ECM) provides a critical structural support for cells and tissues. Defects or changes in the extracellular matrix as a result of excessive deposition or accumulation of ECM components can lead to ECM-mediated diseases or conditions. Among these are collagen-mediated diseases or conditions characterized by the presence of abundant fibrous septae of collagen. Often the only approved treatment for such diseases or conditions is surgery, which can be highly invasive. Other treatments, such as needle aponeurotomy for the treatment of Dupuytren's syndrome or liposuction for cellulite, also are highly invasive.

Bacterial collagenase (also called matrix metalloproteinase-1; MMP-1), an enzyme active at neutral pH that degrades collagen, has been used to treat ECM-mediated conditions such as cellulite (see e.g., published U.S. application serial No. US20070224184); Dupuytren's syndrome (see e.g. U.S. RE39941; U.S. Pat. No. 5,589,171; U.S. Pat. No. 6,086,872); and Peyronie's disease (see e.g., U.S. Pat. No. 6,022,539). Collagenase, however, irreversibly cleaves collagens of type I, II and III. Bacterial collagenase also cleaves type IV collagen, associated with blood vessels, and thus its administration can cause haemorrhage and leaky blood vessels. The prolonged activity of collagenase limits the dosages that can be administered and also risks side effects associated with prolonged activity. Hence, there is a need for alternative treatments of ECM-mediated diseases and conditions. Accordingly, it is among the objects herein to provide alternatives for the treatment of ECM-mediated diseases and conditions.

### SUMMARY

Provided are modified matrix metalloprotease (MMP) enzymes and their use, among others, for treating ECM-mediated diseases or conditions. The enzymes include modified MMPs that are modified to exhibit activity at temperatures different from the unmodified enzymes. Hence, provided are temperature-sensitive mutants of MMP. In particular, the mutants are more active at a lower temperature then a higher temperature and typically are substantially inactive at the higher temperature. For example, the mutants are more active at a temperature that is or is about 25° C. then at a higher temperature that is or is about between 34° C. to 37° C. The mutants also retain an activity of the unmodified enzyme at the lower temperature.

Hence, provided herein are modified matrix metalloproteases (MMP). The MMPs contain one or more modification(s) in the sequence of amino acid residues of an MMP polypeptide or modifications in an allelic or species variant of the MMP, or modifications in a mature form thereof, or a catalytically active fragment of the MMP. The modifications, which are in the primary amino acid sequence, include amino acid replacement(s), insertion(s), deletion(s) and combinations thereof. The MMP can include only one modification, only 2, only 3, 4, 5, 6, 7, 8, 9, 10, 11, 12, 13, 14, 15, 16, 17, 18, 19, 20 or more replacements. The modification be effected on a wildtype MMP, or on an MMP already modified for some other purpose or activity or already mutated. The modification(s) provided herein, confer to the MMP, allelic or species variant thereof or an active fragment thereof, a ratio of enzymatic activity at a permissive temperature compared to at a nonpermissive temperature of at least 1.2, 1.3, 1.4, 1.5, 1.6, 1.7, 1.8, 1.9, 2.0, 2.5, 3.0, 3.5, 4.0, 4.5, 5.0, 6.0, 7.0, 8.0, 9.0, 10.0, 20.0, 30, 40, 50, 60, 70, 80, 90, 100 or more. The MMP can include only 1, only 2, 4, 5, 6, 7, 8, 9, 10, 11, 12, 13, 14, 15, 16, 17, 18, 19, 20 or more replacements to confer a specified ratio of enzymatic activity.

In some embodiments, the modified MMP polypeptide can retain the modified activity of a wildtype MMP at the permissive temperature. For example, it can retain or exhibit at least or about 10%, 20%, 30%, 40%, 50%, 60%, 70%, 80%, 90%, 100%, 110%, 120%, 140%, 150% or more activity.

The modified MMPs include, but are not limited to, collagenases, gelatinases, stromelysins, matrilysins, metalloelastases, enamelysins and membrane-type MMPs, allelic or species variants thereof and active fragments thereof that include such modification. Exemplary MMPs, include those listed in the Tables herein, such as MMP-1 (collagenase-1), MMP-8 (collagenase-2), MMP-13 (collagenase-3), MMP-18 (collagenase-4), MMP-2 (gelatinase A), MMP-9 (gelatinase B), MMP-3 (stromelysin-1), MMP-10 (stromelysin-2), MMP-11 (atromelysin-3; stromelysin-3), MMP-7 (matrilysin), MMP-26 (matrilysin-2), MMP-12 (metalloelastase), MMP-14 (MT1-MMP), MMP-15 (MT2-MMP), MMP-16 (MT3-MMP), MMP-17 (MT4-MMP), MMP-24 (MT5-MMP), MMP-25 (MT6-MMP), MMP-20 (enamelysin), MMP-19, MMP-21, MMP-23, CA-MMP, MMP-27, CMMP and MMP-28 (epilysin). These include allelic variants and species variants as well as active fragments thereof. The allelic and species variants contain the corresponding modification, which readily can be identified, such as by alignment. The active fragment, includes at least one such modification.

The modified MMPs include those that have lower activity at the nonpermissive temperature than the MMP that does not include the modification at the nonpermissive temperature. The permissive temperature can be lower or higher than the nonpermissive temperature. The modified MMPs can have altered activity compared to the unmodified MMP. The activity can be reduced, such as less than 95%, 90%, 80%, 70%, 60%, 50%, 40%, 30%, 25%, 20%, 15%, 10%, 5%, 3%, 1% or less than the activity of the unmodified MMP. The activity also can be increased, such as by the same percentages. Permissive temperatures include, but are not limited to, 21° C., 22° C., 23° C., 24° C., 25° C., 26° C., 27° C., 28° C., 29° C. or 30° C. or about 20° C., 21° C., 22° C., 23° C., 24° C., 25° C., 26° C., 27° C., 28° C., 29° C. or 30° C., such as at or about 25° C. Nonpermissive temperatures include, but are not limited to, 34° C., 35° C., 36° C., 37° C., 38° C. or 39° C. or about 34° C., 35° C., 36° C., 37° C., 38° C. or 39° C. For example, in one embodiment, the nonpermissive temperature is or is about 34° C. or 37° C. and the permissive temperature is 25° C. or about 25° C.

In some embodiments, only a catalytically active fragment is provided or used in any of the methods herein. The catalytically active fragment can be linked, such as fusion protein or chemical conjugate to additional amino acids derived from a different protein, or to another moiety, such as a therapeutic agent. When a catalytically active fragment, such as a catalytic domain is provided, it contains at least one of the amino acid replacements that confer the ratio of enzymatic activity.

Provided herein are modified MMP-1 polypeptides. Exemplary modified MMP-1 polypeptides are any provided herein having a sequence of amino acids set forth in any of SEQ ID NOS:3-705, 779-3458, 3507-3536 or allelic or species variants thereof, zymogen forms, mature forms, or catalytically active fragments thereof.

Among the modified MMPs provided herein that contain a modification that confers a ratio as noted above, are those in which the modification is an amino acid replacement(s), and the replacement(s) is at a position corresponding any one or more positions 84, 85, 95, 98, 99, 100, 103, 104, 105, 106, 109, 110, 111, 112, 118, 123, 124, 126, 147, 150, 151, 152, 153, 155, 156, 158, 159, 170, 171, 176, 178, 179, 180, 181, 182, 183, 185, 187, 188, 189, 190, 191, 192, 194, 195, 197, 198, 206, 207, 208, 210, 211, 212, 218, 223, 227, 228, 229, 230, 233, 234, 237, 240, 251, 254, 255, 256, 257 and 258 in an MMP-1 polypeptide comprising the sequence of amino acids set forth in SEQ ID NO:2 or in corresponding residues in an MMP polypeptide. As described herein, corresponding residues can be identified, for example, using standard alignment programs among proteins with substantial homology.

In particular, provided are modified MMP-1 polypeptides, where the unmodified MMP-1 polypeptide contains the sequence of amino acids set forth in SEQ ID NO:2 or is an allelic or species variant thereof or a mature form thereof that contains an amino acid replacement. Such modifications include, but are not limited to, T84F, E85F, L95K, L95I, R98D, I99Q, E100V, E100R, E100S, E100T, E100F, E100I, E100N, T103Y, P104A, P104M, D105A, D105F, D105G, D105I, D105L, D105N, D105R, D105S, D105T, D105W, D105E, L106C, L106S, A109H, D110A, V111R, D112S, A118T, S123V, N124D, T126S, G147P, R150P, R150V, R150D, R150I, R150H, D151G, N152A, N152S, S153T, F155L, F155A, D156H, D156L, D156A, D156W, D156V, D156K, D156T, D156R, D156M, P158T, P158G, P158K, P158N, G159V, G159T, G159M, G159I, G159W, G159L, G159C, P170D, P170A, G171P, G171E, G171D, A176F, A176W, F178T, F178L, D179N, D179V, D179C, E180Y, E180R, E180T, E180F, E180G, E180S, E180N, E180D, E181T, D181L, D181K, D181C, D181G, E182T, E182Q, E182M, E182G, E183G, R183S, T185R, T185Y, T185H, T185G, T185V, T185Q, T185A, T185E, T185D, N187R, N187M, N187W, N187F, N187K, N187I, N187A, N187G, N187C, N187H, F188V, R189N, R189T, R189Q, E190G, E190Y, E190D, Y191V, N192H, N192S, N192D, N192C, H194P, R195C, R195W, R195L, R195G, R195Q, R195A, R195D, R195V, A197V, A197C, A198G, A198L, A198M, G206A, G206S, L207R, L207V, L207I, L207G, S208R, S208L, S210V, S210A, T211L, D212G, D212H, Y218S, F223C, F223E, F223G, F223A, F223S, F223K, F223M, V227C, V227D, V227E, V227L, V227S, V227W, V227G, V227H, V227Q, V227R, Q228P, L229A, L229T, L29I, A230V, D233E, I234A, I234T, I234E, I234Q, I237L, I237W, I237N, I240S, I240A, I240C, I251 S, I251W, Q254S, T255H, P256C, K257P, K257T and A258P, such as L95K, D105I, D105N, D105L, D105A, D105G, R150P, D156R, D156H, D156K, D156T, G159V, G159T, D179N, E180T, E180F, E182T, T185Q, N187I, A198L, V227E, I234E and I240S, or L95K, D105N, R150P, D156K, D156T, G159V, D179N, E180T, A198L, V227E, and I240S.

Other modified MMP polypeptides are those where the modification is an amino acid replacement(s) and the replacement(s) is at a position corresponding any one or more of positions 95, 105, 150, 151, 155, 156, 159, 176, 179, 180, 181, 182, 185, 187, 195, 198, 206, 210, 212, 218, 223, 227, 228, 229, 230, 233, 234, and 240 in an MMP-1 polypeptide having a sequence of amino acids set forth in SEQ ID NO:2 or in corresponding residues in an MMP polypeptide; where the modification(s) confers to the MMP, allelic or species variant thereof or an active fragment thereof, a ratio of enzymatic activity at a permissive temperature compared to at a nonpermissive temperature of at least 1.5. Such modifications, with reference to MMP-1, include, but are not limited to, L95K, D105A, D105F, D105G, D105I, D105L, D105N, D105R, D105S, D105T, D105W, R150P, D151G, F155A, D156K, D156T, D156L, D156A, D156W, D156V, D156H, D156R, G159V, G159T, A176F, D179N, E180Y, E180T, E180F, D181L, D181K, E182T, E182Q, T185R, T185H, T185Q, T185A, T185E, N187R, N187M, N187F, N187K, N187I, R195V, A198L, A198M, G206A, G206S, S210V, Y218S, F223E, V227C, V227E, V227W, Q228P, L229T, L229I, D233E, I234A, I234T, I234E, I240S, and I240C.

Other modified MMP polypeptides are those where the modification is an amino acid replacement(s) and the replacement(s) is at a position corresponding any one or more positions 95, 105, 150, 156, 159, 179, 180, 182, 185, 187, 195, 198, 212, 223, 227, 234, and 240 in an MMP-1 polypeptide having a sequence of amino acids set forth in SEQ ID NO:2 or in corresponding residues in an MMP polypeptide; and the modified MMP polypeptide retains at least or about 30%, 40%, 50%, 60%, 70%, 80%, 90%, 100%, 110%, 120%, 140%, 150% or more activity at 25° C. compared to wildtype MMP-1 at 25° C. This includes modified MMP polypeptides where a modification is selected from among L95K, D105A, D105G, D105I, D105L, D105N, D105S, D105W, D105T, R150P, D156K, D156T, D156V, D156H, D156R, G159V, G159T, D179N, E180Y, E180T, E180F, E182T, T185H, T185Q, T185E, N187M, N187K, N187I, R195V, A198L, F223E, V227E, I234E and I240S.

Among the modified MMP polypeptides are those in which the activity of the polypeptide is reversible upon exposure to the nonpermissive temperature, such as, for example, where upon exposure to the nonpermissive temperature and return to the permissive temperature the polypeptide exhibits at or about 120%, 125%, 130%, 140%, 150%, 160%, 170%, 180%, 200% or more or the activity compared to at the nonpermissive temperature. These include modified MMP polypeptides where the modification is an amino acid replacement(s) and the replacement(s) is at a position corresponding to any one or more positions D105A, D105F, D105G, D105S, D105T, R150P, G159T, E180Y, E180T, E180F, T185H, T185Q, T185A, T185E, N187R, N187M, N187K, R195V, A198L, A198M, S210V, Y218S, F223E, V227W, L229I and I240C in an MMP polypeptide.

Among the modified MMP polypeptides are those in which the activity of the polypeptide is irreversibly inactive upon exposure to the nonpermissive temperature, such as for example, modified MMP polypeptides, that, upon exposure to the nonpermissive temperature and return to the permissive temperature the polypeptide, exhibit at or about 50%, 60%, 70%, 80%, 90%, 100%, 105%, 110%, 115%, or less than 120% the activity at the non-permissive temperature. These include, but are not limited to, modified MMP polypeptides with a modification in an MMP polypeptide selected from among L95K, D105I, D105L, D105N, D105R, D105W, D151G, F155A, D156K, D156T, D156L, D156A, D156W, D156V, D156H, D156R, G159V, A176F, D179N, D181L, D181K, E182T, E182Q, T185R, N187F, N187I, G206A, G206S, V227C, V227E, Q228E, L229T, D233E, I234A, I234T, I234E and I240S.

Any of the modified MMP-1 polypeptides provided herein above can further include an activity mutation, whereby the mutation confers increased activity compared to the MMP-1 not containing the modification. For example, such a modified MMP-1 polypeptide can include amino acid replacement(s) at a position corresponding to any one or more of positions 81, 84, 85, 86, 87, 89, 104, 105, 106, 107, 108, 109, 124, 131, 133, 134, 135, 143, 146, 147, 150, 152, 153, 154, 157, 158, 160, 161, 164, 166, 167, 180, 183, 189, 190, 207, 208, 211, 213, 214, 216, 218, 220, 222, 223, 224, 225, 226, 227, 228, 229, 230, 231, 232, 235, 236, 238, 239, 244, 249, 254, 256, 257 and 258 in an MMP-1 polypeptide comprising the sequence of amino acids set forth in SEQ ID NO:2. For example, amino acid replacement can be F81L, F81A, F81G, F81Q, F81R, F81H, T84H, T84L, T84D, T84R, T84G, T84A, E85S, E85V, G86S, N87P, N87R, N87G, N87Q, R89A, R89T, R89G, R89K, P104E, P104D, P104Q, D105V, L106V, P107T, P107S, P107A, R108E, R108A, R108K, R108S, A109S, A109R, A109G, A109M, A109V, N124G, T131D, K132R, V133T, V133L, S134E, S134D, E135M, S143I, R146S, G147R, G147F, R150E, R150G, R150M, T150T, R150A, R150N, R150K, R150L, R150V, R150D, N152G, N152F, N152L, N152I, S153T, S153P, S153F, S153D, S153Y, P154S, P154I, G157F, P158V, P158I, G160Q, N161L, N161R, N161Y, N161E, N161T, N161I, N161V, N161F, N161Q, H164S, F166W, Q167R, Q167A, Q167S, Q167F, Q167P, Q167T, Q167V, Q167M, E180D, R183S, R189N, R189T, R189Q, E190D, L207M, S208K, S208R, S208L, T211N, I213G, G214L, G214E, L216I, Y218W, S220R, S220A, S220Q, S220T, S220G, S220M, S220V, S220N, T222R, T222P, T222S, T222F, T222N, F223Y, F223H, 2224Q, S224K, S224D, G225Q, G225E, G225H, D226S, D226E, D226P, D226I, V227T, Q228A, Q228D, Q228E, Q228G, Q228H, Q228K, Q228L, Q228M, Q228N, Q228R, Q228S, Q228T, Q228W, Q228Y, L229Q, L229P, L229V, A230G, A230W, A230D, A230I, A230S, A230C, A230V, A230T, A230M, A230N, A230H, Q231I, Q231A, Q231F, Q231D, Q231G, Q231V, Q231W, Q231S, Q231H, Q231M, D232H, D232G, D232R, D232P, D232Y, D232S, D232F, D232V, D232K, D232W, D232Q, D232E, D232T, D232L, D235G, D235A, D235L, D235E, D235R, D235Q, D235T, D235N, G236M, G236R, G236S, G236T, G236C, G236K, G236E, G236L, G236N, Q238T, A239S, A239V, A239L, A239I, A239G, A239K, A239H, A239R, S244W, S244Q, Q249W, Q254S, P256S, K257E, K257R, or A258P. Exemplary modified MMP-1 polypeptides containing at least one temperature sensitive mutant and at least one activity mutant include those having amino acid replacements S208K/G159V; S208K/D179N; S208K/V227E; G214E/G159V; G214E/D179N; and I213G/D179N.

Also provided herein are modified MMP-1 polypeptides that are activity mutants, whereby the modified MMP-1 polypeptide exhibits increased activity compared to the MMP-1 not containing the modification. Exemplary activity mutants are any having an amino acid replacement in the above paragraph, and further herein in Section D.2.

MMPs that can be modified include, but are not limited to, MMP-1, MMP-8, MMP-13, MMP-18, MMP-2, MMP-9, MMP-3, MMP-10, MMP-7, MMP-6, MMP-12, and allelic or species variants, mature forms, or catalytically active fragments thereof. Exemplary modified MMPs include any in which the unmodified MMP polypeptide has a sequence of amino acids set forth in any of SEQ ID NOS: 1, 711, 714, 717, 720, 723, 726, 729, 732, 735, 738, 741, 744, 747, 750, 753, 756, 759, 762, 765, 768, 771, 774 or 777, zymogen forms, allelic or species variants thereof or active fragments thereof. Such modified MMPs can have a modification at a corresponding position in the MMP compared to any of the modifications in MMP-1 provided herein. Exemplary of such corresponding positions are set forth in FIGS. 2 and 3, and exemplary mutations set forth in Section D herein. These include, for example polypeptides containing amino acid replacement(s) at a position corresponding to any two or more positions 95, 105, 151, 156, 159, 176, 179, 180, 181, 182, 185, 195, 198, 206, 210, 212, 218, 223, 228, 229, 233, 234, and 240 in an MMP-1 polypeptide having a sequence of amino acids set forth in SEQ ID NO:2 or in corresponding residues in an MMP polypeptide.

Provided are modified MMP polypeptides with two or more modifications, where at least one of the modifications confers the ratio, or where two do so, or more do so. The modified MMP polypeptides can contain 3, 4, 5, 6, 7, 8, 9, 10, 11, 12, 13, 14, 15, 16, 17, 18, 19, 20 or more modifications. Some or all of these can confer or contribute to a desired ratio of activity between the permissive and non-permissive temperature. Exemplary of modified MMP polypeptides are those that contain two or more amino acid replacement(s) and the replacement(s) are at a position corresponding to any two or more of positions 95, 105, 150, 156, 159, 179, 180, 182, 185, 187, 198, 227, 234 and 240 in an MMP-1 polypeptide having a sequence of amino acids set forth in SEQ ID NO:2 or in corresponding residues in an MMP polypeptide, such as, for example, where the two or more modifications in an MMP polypeptide are selected from among L95K, D105N, R150P, D156K, D156T, G159V, D179N, E180T, A198L, V227E, and I240S, or any where the two or more modifications in an MMP polypeptide are selected from among any set forth in Table 15.

As noted, the modified MMP polypeptide can be a zymogen, an active enzyme, can contain only a catalytically active fragment, such as the catalytic active domain, or can lack all or a portion of a proline rich linker and/or a hemopexin domain.

The modified MMP polypeptides can contain one or more additional modifications in addition to those that confer the activity ratio, such as, but not limited to, modifications that confer increased stability, increased half-life, altered substrate specificity and/or increased resistance to inhibitors. For example, the modified MMP polypeptide can be glycosylated as expressed or can be modified to be glycosylated, or can contain other modifications, such as PEGylation. The modified MMP polypeptide can be a fusion protein with another protein, such as an Fc fusion, or it can be provided as a dimer or a heterodimer or other multimer.

Also provided are nucleic acid molecules and/or vectors that encode any of the modified MMP polypeptides. Vectors include prokaryotic, viral and eukaryotic vectors, including mammalian vector and yeast vectors, such as, for example, adenovirus, an adeno-associated virus, a retrovirus, a herpes virus, a lentivirus, a poxvirus, a cytomegalovirus and Pichia vectors and artificial chromosomes. Cells, including prokaryotic, such as bacterial and algael cells, and eukaryotic, such as mammalian cells, containing the vectors are provided. The cells can express the modified MMP polypeptide, which can be encoded by nucleic acid that directs its secretion or trafficking to other loci in a cell. Methods for producing the MMPs by expressing the encoded MMP in a cell are provided. The MMPs provided herein can be provided in lyophilized or other dried or non-liquid forms.

Also provided are compositions, including pharmaceutical compositions, containing any or mixtures of the modified MMP polypeptides. The pharmaceutical compositions can be formulated for treatment of any disease amenable to treatment by an MMP, and particularly in the methods provided herein, for treatment of disease or conditions of the extracellular matrix (ECM). The compositions can be formulated for single dosage administration and contain multiple dosages or can require dilution or addition of other agents. Amounts per dosage, include for example, 10 μg to 100 mg, 50 μg to 75 mg, 100 μg to 50 mg, 250 μg to 25 mg, 500 μg to 10 mg, 1 mg to 5 mg, or 2 mg to 4 mg per dosage.

Also provided are uses of the modified MMPs for treating a disease or condition of the ECM or formulation of a medicament therefore, and methods for treating a disease or condition of the extracellular matrix (ECM), and processes for treating a disease or condition of the ECM. In practicing the methods, the MMP polypeptide or pharmaceutical compositions containing the MMP polypeptide is administered to the ECM with an activator that when administered or provided to the ECM, provides a temperature activating condition for the enzyme such that the MMP is active. The modified MMP polypeptide is more active at a permissive temperature then at the nonpermissive physiologic temperature, and the activating condition is not present in the ECM prior to administration of the activator.

Also provided herein are methods for treating a disease or condition of the ECM by administering to the ECM a modified MMP-1 polypeptide or composition thereof, or other modified MMP, that exhibits temperature sensitivity, whereby the modified MMP-1 exhibits activity at a permissive temperature that is below the physiologic temperature of the body. In the method, the MMP-1 is administered at or below the permissive temperature. The modified MMP-1 can be mixed with a composition that is at or below the permissive temperature immediately before administration or it can be provided in a composition that is at or below the permissive temperature. In the methods, prior to administration, the ECM can be cooled to below the physiological temperature of the body, for example, by using a cold pack administered at the locus of administration of the MMP. Further, conditional activation of the MMP can be controlled for a predetermined time. For example, the ECM can be maintained at below the physiological temperature of the body for a predetermined time.

Also provided herein are methods similar to above, whereby the modified MMP is active at a permissive temperature that is above the physiologic temperature of the body. Hence, the MMP, when administered at or above the permissive temperature, can be mixed with a composition that is at or above the permissive temperature immediately before administration or it can be provided in a composition that is at or above the permissive temperature. Conditional activation can be achieved by exposure of the locus of administration by heat to warm the ECM. This can be for a predetermined time.

In the methods, uses and processes herein, the MMP can be a zymogen that is processed, such by a processing agent, before administration. Processing agents include, but are not limited to, plasmin, plasma kallikrein, trypsin-1, trypsin-2, neutrophil elastase, cathepsin G, tryptase, chymase, proteinase-3, proteinase-3, furin, urinary plasminogen activator (uPA), an active MMP, 4-aminophenylmercuric acetate (AMPA), HgCl₂, N-ethylmaleimide, sodium dodecyl sulfate (SDS), chaotropic agents, oxidized glutathione, reactive oxygen, Au(I) salts, acidic pH and heat. The modified MMP includes any provided herein, including, but are not limited to, modified MMP-1, MMP-2, MMP-3, MMP-7, MMP-10, MMP-26 and MT1-MMP. The processing agent is purified away from the modified MMP polypeptide before administration as can any non-active cleavage products of the MMP polypeptide. The modified MMP polypeptide is administered in an amount to treat the disease or condition under the activating conditions (i.e., during the period when it is exposed to the permissive temperature). The activator can be administered or provided prior to, simultaneously, subsequently or intermittently from the MMP. Exemplary activator include, a hot pack or a cold pack, a hot or cold liquid, buffer or solution, such as provision of the MMP in chilled buffer, wherein the chilled buffer is the activator. The buffer can be chilled to 4° C., 5° C., 6° C., 7° C., 8° C., 9° C., 10° C., 11° C., 12° C., 13° C., 14° C., 15° C., 16° C., 17° C., 18° C., 19° C., 20° C. or more or about any of these temperatures.

Administration can be effected by any suitable route, including but not limited to, subcutaneous, intramuscular, intralesional, intradermal, topical, transdermal, intravenous, oral and rectal administration, such as for example, sub-epidermal administration, including, subcutaneous administration.

The modified MMP polypeptide can be administered simultaneously, intermittently, sequentially or in the same composition with other active agents, such as a pharmacologic agent, including, for example, a small molecule drug compound (i.e., a compound that is not a macromolecule or biomolecule), dispersing agents, anesthetics and vasoconstrictors and combinations thereof. Exemplary of dispersing agents is a hyaluronan-degrading enzyme, such as, for example, a hyaluronidase. Exemplary of hyaluronidases is PH20, such as a soluble truncated form thereof, including, a hyaluronidase that contains or has a sequence of amino acids set forth in SEQ ID NO:3475, or an allelic or species variant or other variant thereof, including those having at least 60%, 70%, 80%, 90%, 91%, 92%, 93%, 95% 95%, 96%, 97%, 98%, 99% or more sequence identity to the sequence of amino acids set forth in SEQ ID NO:3475, such as 91% or greater sequence identity. The hyaluronidase can be one that is glycosylated. The anesthetics include any suitable anesthetic, such as, for example, lidocaine. The vasoconstrictor can be any suitable vasoconstrictor, such as an alpha adrenergic receptor agonist, such as, for example, levonordefrin, epinephrine or norepinephrine. In the methods, the other agent can be administered prior to administration of the MMP.

The ECM component that is affected by the treatment can include, for example, a collagen, an elastin, a fibronectin or a proteoglycan. The component affected depends upon the MMP selected. Where the ECM component is collagen, the collagen can be selected from among type I, type II, type III or type IV collagen. In any embodiment, the MMP is selected to be one that degrades a particular target, such as selection of a collagenase where the target is collagen. Mixtures of MMP can be used to degrade a plurality of ECM components. Diseases and conditions treated include, collagen-mediated diseases or conditions, such as, but not limited to, cellulite, Dupuytren's disease, Peyronie's disease, Ledderhose fibrosis, stiff joints, existing scars, scleroderma, lymphedema and collagenous colitis, herniated discs, stiff joints, such as a frozen shoulder, scars, such as a scar resulting from among surgical adhesions or keloids, hypertrophic scars and depressed scars.

Also provided are combinations of any modified MMP polypeptide provided herein and an activator thereof. Also provided are kits containing the combinations and one or more of a device for administration and, optionally instructions for administration, and other containers and components, such as reducing agents that increase activity, such as for enzyme with free sulfhydryl groups.

### BRIEF DESCRIPTION OF THE FIGURES

FIG. 1 is an alignment of zymogen MMPs, indicating the propeptide, the catalytic domain, linker region, hemopexin domains 1-4, fibronectin type II repeats, the basic region, the cysteine switch, the calcium (Ca) binding sites I and II, and the zinc binding site. The alignment includes zymogen MMPs, including MMP-1 (SEQ ID NO:2), MMP-8 (amino acids 21-467 of SEQ ID NO:711), MMP-13 (amino acids 20-471 of SEQ ID NO:714), MMP-18 (amino acids 18-467 of SEQ ID NO:717), MMP-2 (amino acids 30-660 of SEQ ID NO:720), MMP-9 (amino acids 20-707 of SEQ ID NO:723), MMP-3 (amino acids 18-477 of SEQ ID NO:726), MMP-10 (amino acids 18-476 of SEQ ID NO:729), MMP-11 (amino acids 32-488 of SEQ ID NO:732), MMP-7 (amino acids 18-267 of SEQ ID NO:735), MMP-26 (amino acids 18-261 of SEQ ID NO:738), MMP-12 (amino acids 17-470 of SEQ ID NO:741), and MMP-19 (amino acids 19-508 of SEQ ID NO:765). A “*” means that the residues or nucleotides in that column are identical in all sequences in the alignment, a “:” means that conserved substitutions have been observed, and a “.” means that semi-conserved substitutions are observed.

FIG. 2 is an alignment of the catalytic domains of exemplary MMPs, indicating exemplary conserved and conservative amino acid residues. It is understood that other conserved and conservative amino acid residues exist between and among MMPs. Thus, this figure and identification of residues is not intended to limit corresponding residues between and among MMPs. The exemplary MMPs include: MMP-1 (amino acids 81-242 of SEQ ID NO:2), MMP-8 (amino acids 101-242 of SEQ ID NO:711), MMP-13 (amino acids 104-248 of SEQ ID NO:714), MMP-18 (amino acids 100-246 of SEQ ID NO:717), MMP-2 (amino acids 110-417 of SEQ ID NO:720), MMP-9 (amino acids 94-425 of SEQ ID NO:723), MMP-3 (amino acids 100-247 of SEQ ID NO:726), MMP-10 (amino acids 99-246 of SEQ ID NO:729), MMP-11 (amino acids 98-228 of SEQ ID NO:732), MMP-7 (amino acids 95-242 of SEQ ID NO:735), MMP-26 (amino acids 90-236 of SEQ ID NO:738), MMP-12 (amino acids 106-247 of SEQ ID NO:741), and MMP-19 (amino acids 98-239 of SEQ ID NO:765). Exemplary conserved and conservative positions between and among MMPs are highlighted.

FIG. 3 is an alignment similar to that depicted in FIG. 2. In the alignment, exemplary conserved and conservative positions corresponding to MMP-1 activity mutants are highlighted between and among other MMPs.

### DETAILED DESCRIPTION

Outline

A. Definitions
B. Overview—Temperature Sensitive Matrix Metalloproteases
C. Matrix Metalloproteases and the Extracellular Matrix
   1. The Extracellular Matrix
      a. Components of the ECM
         i. Collagens
         ii. Elastin
         iii. Fibronectin
         iv. Glycosaminoglycans (GAGs)
         1) Proteoglycans
         2) Hyaluronic Acid
      b. Histology of the Skin
         i. The Epidermis
         ii. The Dermis
         iii. The Hypodermis
      c. Diseases of the ECM
   2. Matrix Metalloproteases
      a. Function
      b. Structure and Activation
      3. Matrix Metalloprotease 1 (MMP-1)
D. Modified Matrix Metalloprotease-1 Polypeptides
   1. Temperature-Sensitive Matrix Metalloprotease-1 (tsMMP-1 Mutants)
      Exemplary Temperature Sensitive Modifications
   2. Matrix Metalloprotease Activity Mutants
   3. Combinations
   4. Additional Modifications
   5. Other MMPs
E. Methods of Producing Nucleic Acids Encoding tsMMPs, and Polypeptides Thereof
   1. Vectors and Cells
   2. Expression
      a. Prokaryotic Cells
      b. Yeast Cells
      c. Insect Cells
      d. Mammalian Cells
      e. Plants
   3. Purification Techniques
F. Preparation, Formulation and Administration of tsMMPs
   1. Injectables, solutions and emulsions
      Lyophilized Powders
   2. Topical Administration
   3. Compositions for other routes of administration
   4. Activator
   5. Combination Therapies
      Hyaluronidases
G. Packaging and Articles of Manufacture of tsMMPs
   1. Single Chamber Apparatus
   2. Dual Chamber Apparatus
   3. Kits
H. Methods of Assessing Activity of tsMMPs
   1. Methods of Assessing Enzymatic Activity
   2. Methods of Assessing ECM Degradation
      a. In vitro assays
      b. In vivo assays
      c. Non-human animal models
I. Exemplary Methods of Treating Diseases or Defects of ECM
   1. Collagen-Mediated Diseases or Conditions
      a. Cellulite
      b. Dupuytren's Disease
      c. Peyronie's Disease
      d. Ledderhose Fibrosis
      e. Stiff Joints
      f. Existing Scars
         i. Surgical Adhesions
         ii. Keloids
         iii. Hypertrophic scars
         iv. Depressed Scars
      g. Scleroderma
      h. Lymphedema
      i. Collagenous colitis
   2. Spinal Pathologies
J. Examples

### A. DEFINITIONS

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, the extracellular matrix (ECM) refers to a complex meshwork structure that surrounds and provides structural support to cells of specialized tissues and organs. The ECM is made up of structural proteins such as collagen and elastin; specialized proteins such as fibronectin; and proteoglycans. The exact biochemical composition varies from tissue to tissue. In the skin, for example, it is the dermal layer that contains the ECM. Reference to the “interstitium” is used interchangeably herein to refer to the ECM.

As used herein, components of the ECM refers to any material produced by cells of connective tissue and secreted into the interstitium. For purposes herein, reference to ECM components refers to proteins and glycoproteins, and not to other cellular components or other components of the ECM. Exemplary ECM components include, but are not limited to, collagen, fibronectin, elastin and proteoglycans.

As used herein, a matrix degrading enzyme refers to any enzyme that degrades one or more components of the ECM. Matrix-degrading enzymes include proteases, which are enzymes that catalyze the hydrolysis of covalent peptide bonds. Matrix-degrading enzyme include any known to one of skill in the art. Exemplary matrix-degrading enzymes include matrix metalloproteases, allelic or species variants or other variants thereof.

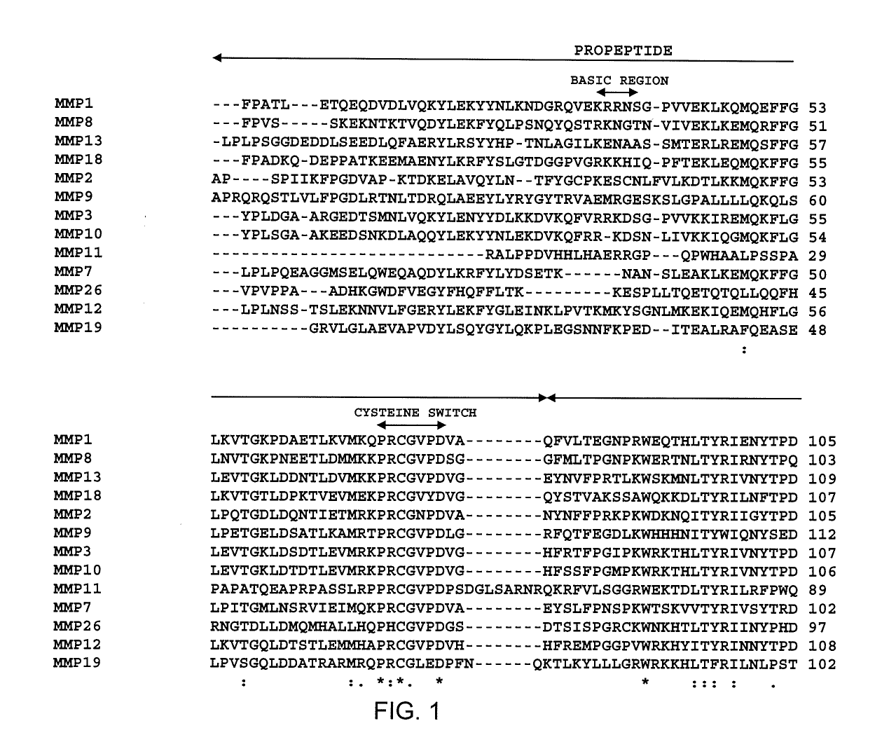

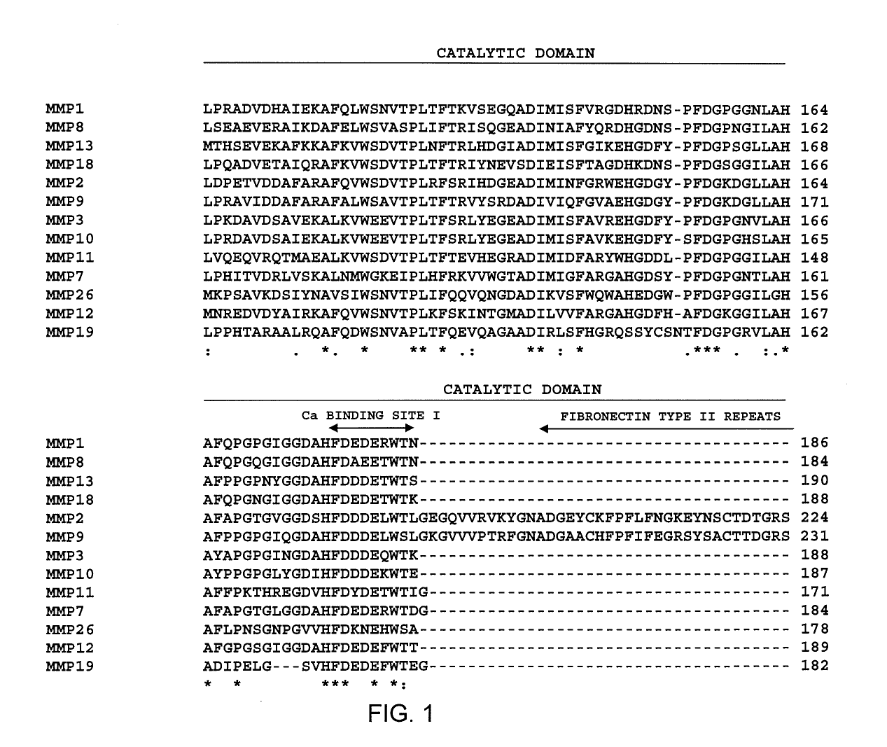

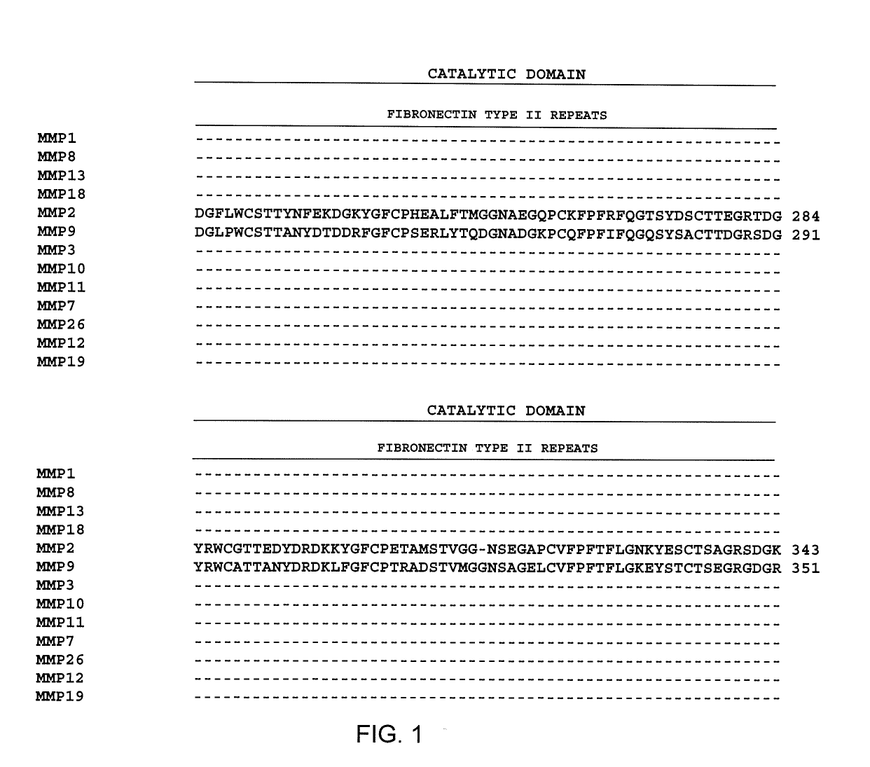

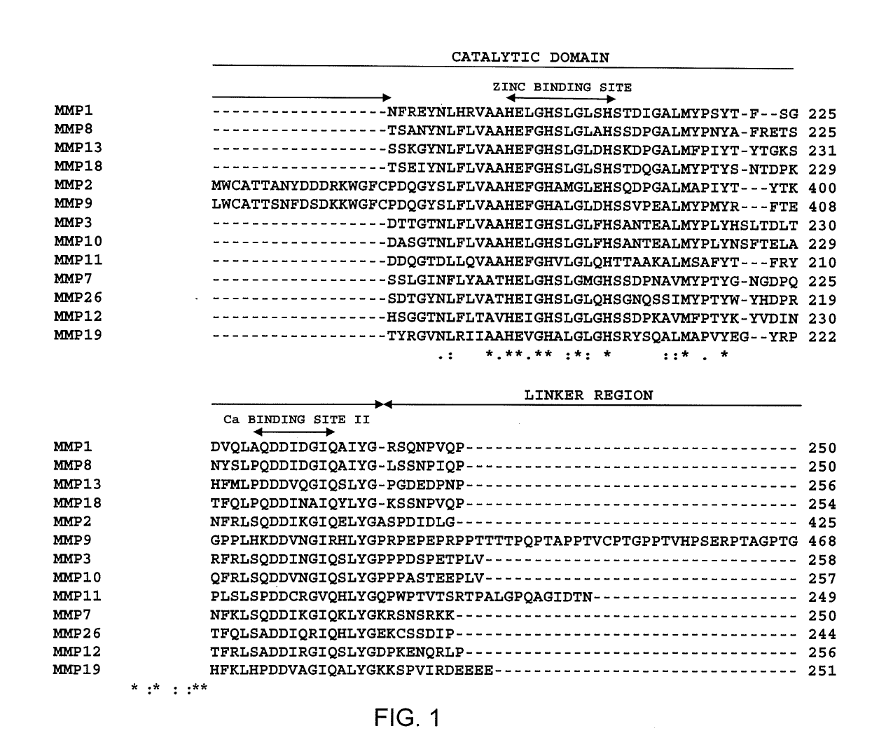

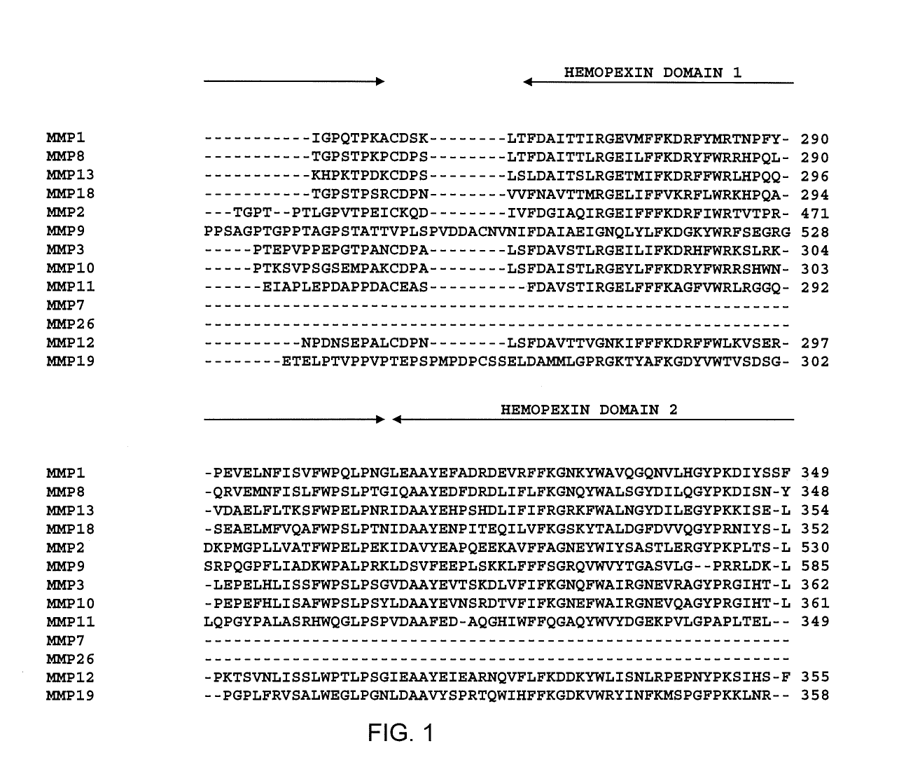

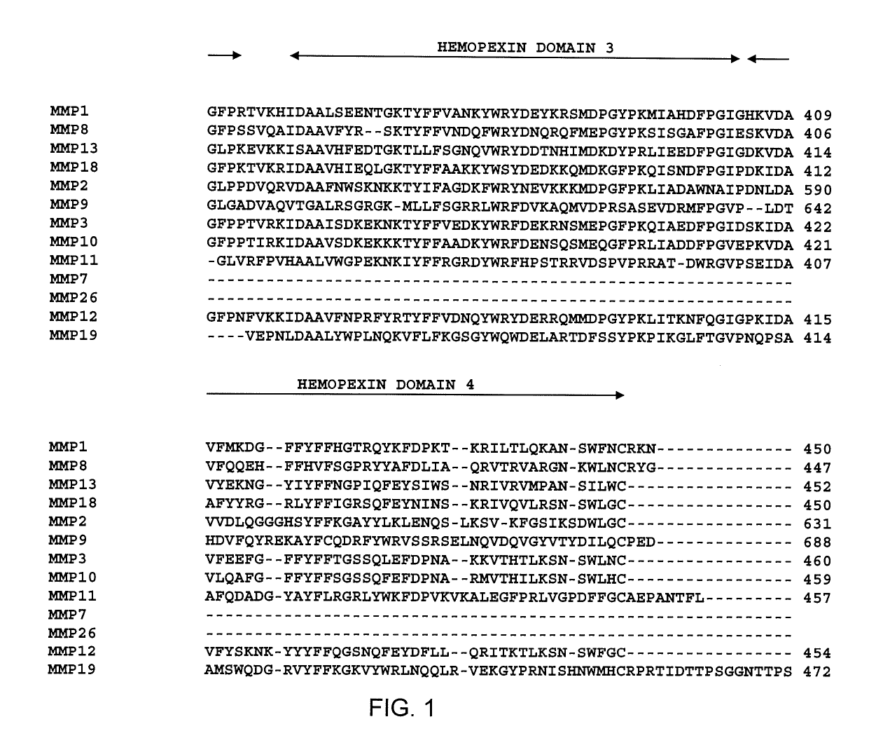

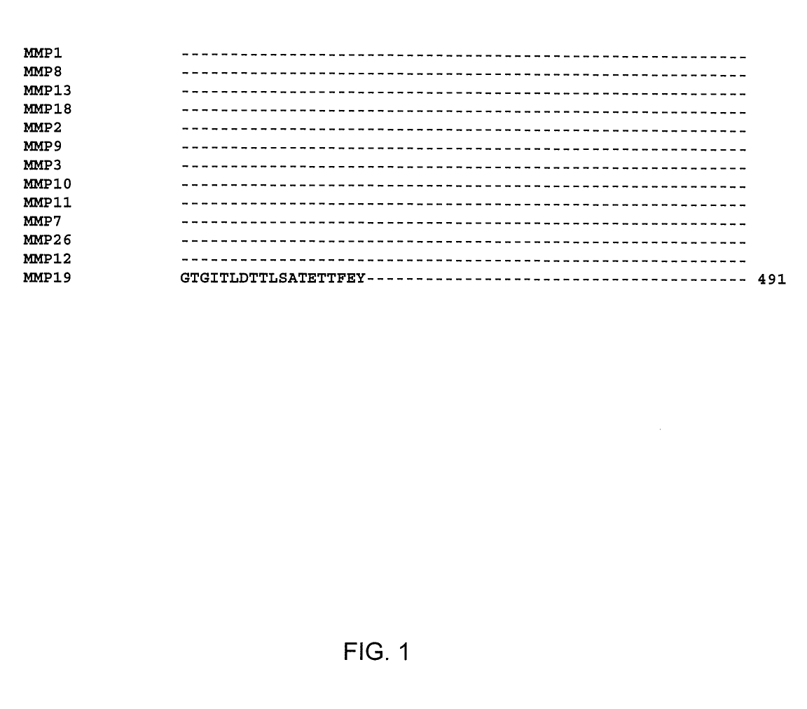

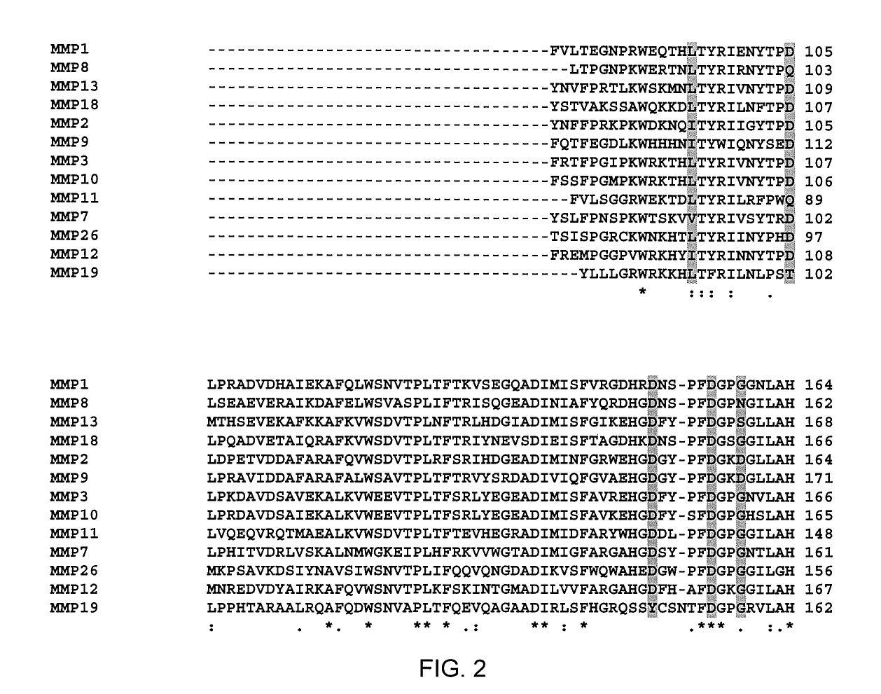

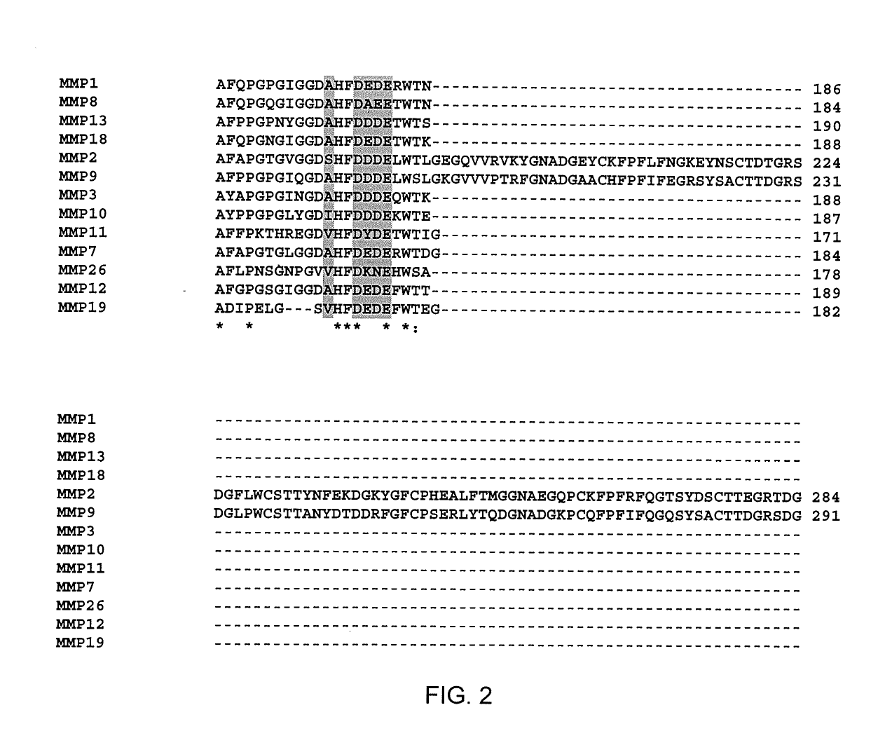

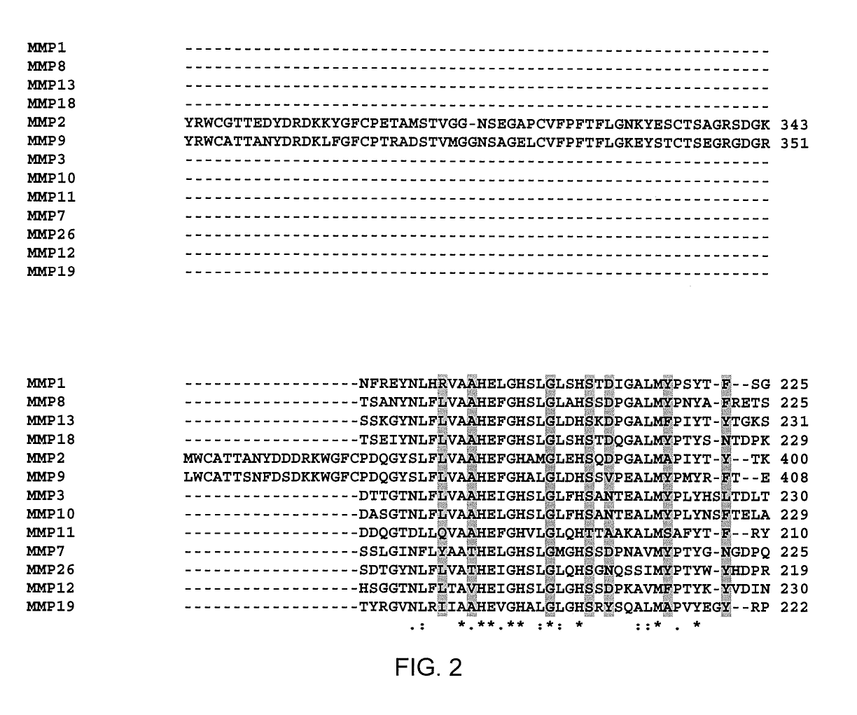

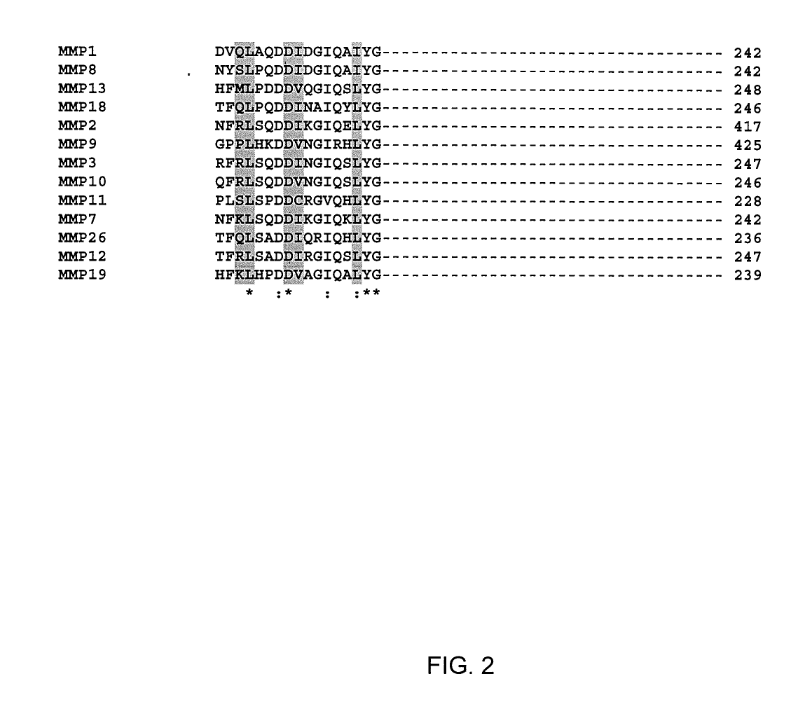

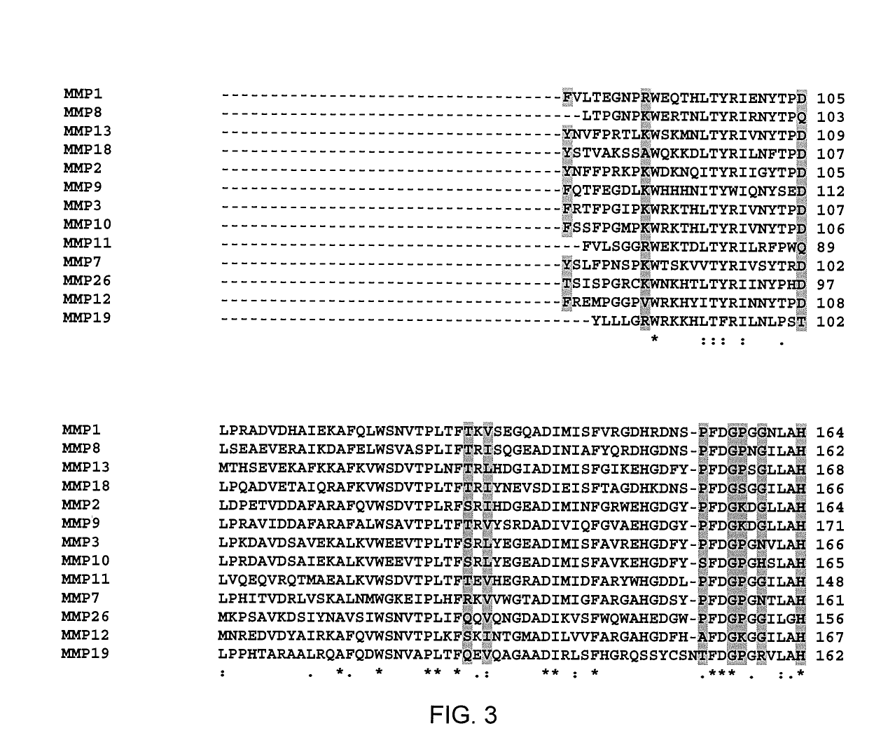

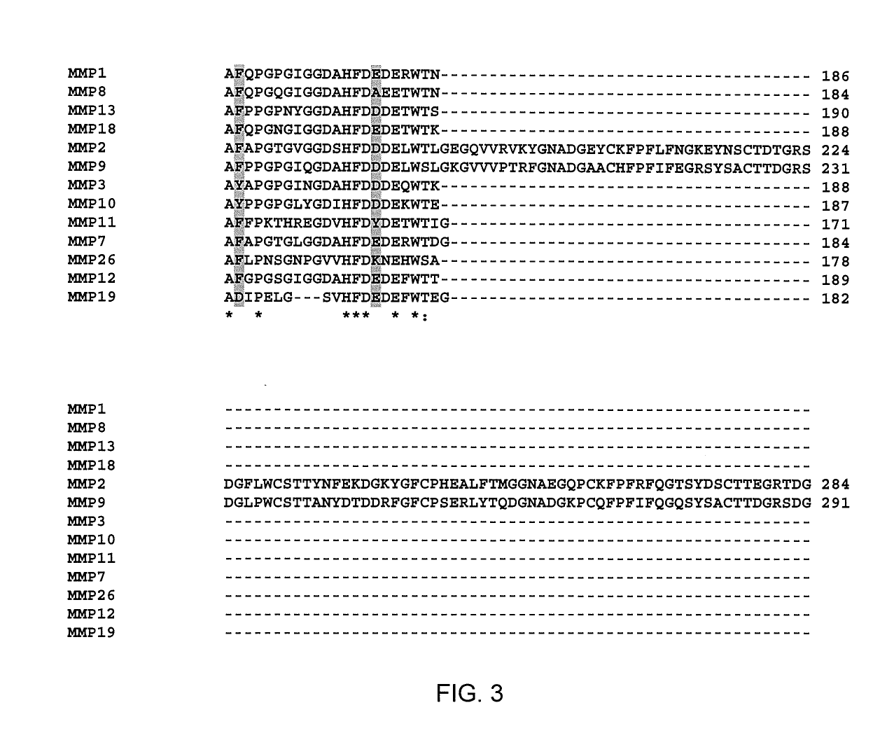

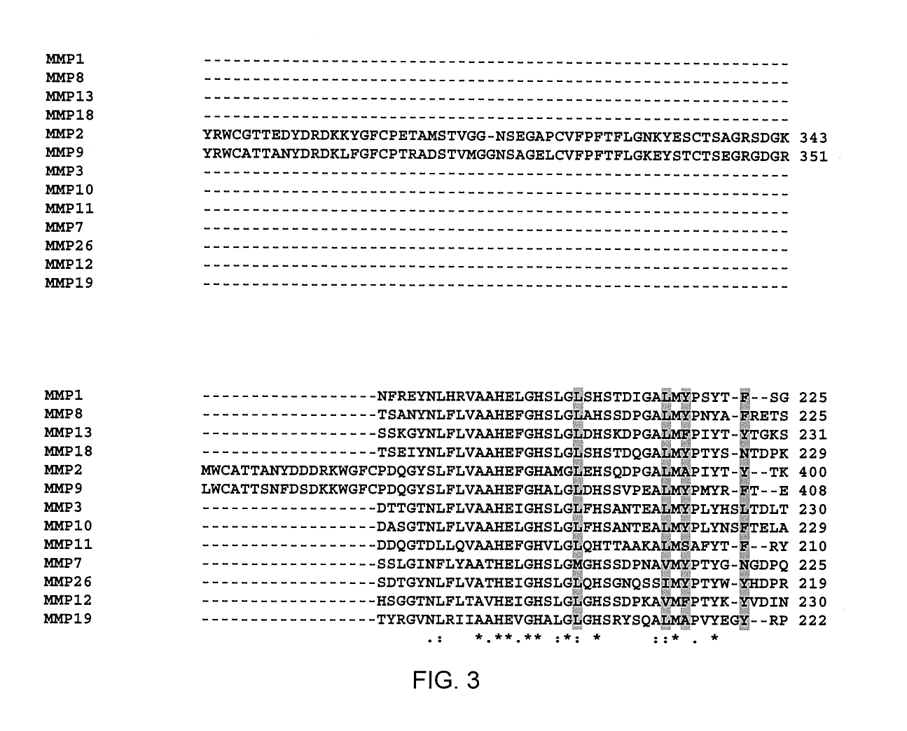

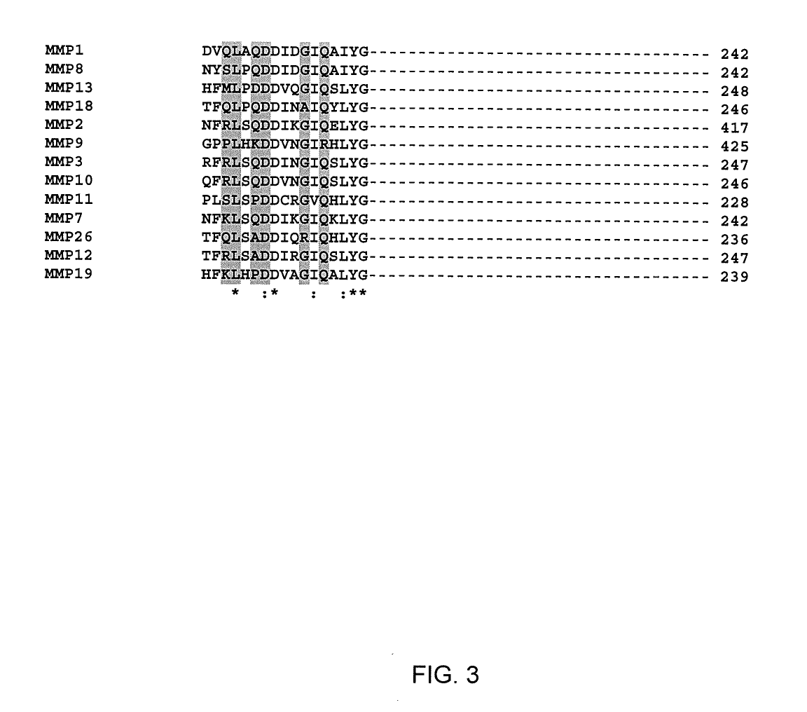

As used herein, a matrix metalloprotease (MMP) refers to a type of matrix degrading enzyme that is a zinc-dependent endopeptidase that contain an active site Zn²⁺ required for activity. MMPs include enzymes that degrade components of the ECM including, but not limited to, collagen, fibronectin, elastin and proteoglycans. MMPs generally contain a propeptide, a catalytic domain, a proline linker and a hemopexin (also called haemopexin-like C-terminal) domain. Some MMPs contain additional domains. Exemplary MMPs are set forth in Table 5. Reference to an MMP includes all forms, for example, the precursor form (containing the signal sequence), the proenzyme form (containing the propeptide), the processed active form, and forms thereof lacking one or more domains. For example, reference to an MMP refers to MMPs containing only the catalytically active domain. Domains of exemplary MMPs are identified in FIG. 1. MMPs also include allelic or species variants or other variants thereof.

As used herein, a modified matrix degrading enzyme or a modified MMP (also interchangeably referred to as a variant or mutant) refers to an enzyme that has one or more modifications in primary sequence compared to a wildtype enzyme. The one or more mutations can be one or more amino acids replacements (substitutions), insertions, deletions, and any combination thereof. A modified enzyme includes those with 1, 2, 3, 4, 5, 6, 7, 8, 9, 10, 11, 12, 13, 14, 15, 16, 17, 18, 19, 20 or more modified positions. The modifications can provide altered properties of the enzyme. Exemplary of modifications include those described herein that confer temperature-sensitive activity of the enzyme. Other modifications include those that confer altered substrate specificity, stability and/or sensitivity to inhibitors (e.g. TIMPs). A modified enzyme typically has 60%, 70%, 80%, 85%, 90%, 91%, 92%, 93%, 94%, 95%, 96%, 97%, 98%, 99% or more sequence identity to a corresponding sequence of amino acids of a wildtype enzyme. Typically, a modified enzyme retains an activity or sufficient activity (e.g. degradation of an ECM component) of a wildtype enzyme. It is understood that modifications conferring temperature sensitivity retain an activity or sufficient activity at the requisite temperature compared to a wildtype enzyme at the physiologic temperature.

As used herein, an activity mutant or mutation or variant or modification refers to a modified enzyme, for example a modified matrix metalloprotease such as a modified MMP-1, that exhibits increased enzymatic activity compared to the enzyme that does not contain the particular modification. For example, the enzyme exhibits 1.2-fold to 100-fold or higher increased enzymatic activity, for example, 1.2-fold, 1.3, 1.4, 1.5, 1.6, 1.7, 1.8, 1.9, 2.0, 3.0, 4.0, 5.0, 6.0, 7.0, 8.0, 9.0, 10, 20, 30, 40, 50, 60, 70, 80, 90, 100-fold or more increased enzymatic activity. It is understood that in determining enzymatic activity, the enzymatic activity of the mutant and the unmodified enzyme (e.g. wildtype) is measured under the same assay conditions. Reference to an activity mutant herein is not dependent on temperature. For example, an activity mutant provided herein can exhibit increased activity compared to the enzyme that does not contain the modification at both the permissive and nonpermissive temperature.

As used herein, a temperature sensitive (ts) mutant or mutation or variant or modification conferring temperature sensitivity refers to a polypeptide that is modified to exhibit higher enzymatic activity at some temperatures called permissive temperatures compared to other temperatures called nonpermissive temperatures. Generally, a temperature-sensitive mutant exhibits higher enzymatic activity at lower temperatures then at higher temperatures.

As used herein, permissive temperature is the temperature at which a polypeptide exhibits a higher enzymatic activity then at a second temperature called the nonpermissive temperature. Hence, the modified enzymes provided herein exhibit different activities at different temperatures that is higher at one temperature then at another temperature. The temperature at which it exhibits more activity is the permissive temperature. For example, the permissive temperature is a temperature that is below the physiological temperature of the body, for example, 18° C. to 30° C., and in particular 20° C. to 25° C. Hence, the enzyme exhibits increased activity at a temperature below the physiological temperature of the body then activity at the physiological temperature of the body, such as exists in the interstitium. For example, the permissive temperature is or is about 18° C. 19° C., 20° C., 21° C., 22° C., 23° C., 24° C., 25° C., 26° C., 27° C., 28° C., 29° C. or 30° C.

As used herein, a nonpermissive temperature is the temperature where a polypeptide exhibits lower enzymatic activity then at the permissive temperature and exhibits reduced activity compared to the enzyme that is not modified. Temperature-sensitive mutants provided herein exhibit enzymatic activity at the nonpermissive temperature that is at or about 1%, 2%, 3%, 4%, 5%, 6%, 7%, 8%, 9%, 10%, 20%, 30%, 40%, 50%, 60%, 70%, 80% or 90% up to less then 100% the activity at the permissive temperature. The temperature sensitive mutants provided herein also exhibit 1%, 2%, 3%, 4%, 5%, 6%, 7%, 8%, 9%, 10%, 20%, 30%, 40%, 50%, 60%, 70%, 80% or 90% up to less then 100% of the activity at the nonpermissive temperature compared to the enzyme that is not modified (e.g. wildtype enzyme) at the nonpermissive temperature. For example, the nonpermissive temperature is a temperature that is near to, at or above the physiological temperature of the body, for example, 32° C. to 39° C., for example, 32° C., 33° C., 34° C., 35° C., 36° C., 37° C., 38° C., or 39° C.

As used herein, the ratio of enzymatic activity at the permissive temperature compared to the nonpermissive temperature refers to the relation of enzymatic activity at the permissive and nonpermissive temperatures. It is expressed by the quotient of the division of the activity at the permissive temperature by the activity at the nonpermissive temperature. It is understood that in determining enzymatic activity and the ratio of enzymatic activity, the enzymatic activity at the permissive and nonpermissive temperatures is measured under the same assay conditions, except for the difference in temperature.

As used herein, physiological temperature refers to temperature conditions maintained in the body, which is approximately 37° C., for example, at or about 34° C., 35° C., 36° C., 37° C., 38° C. or 39° C. It is understood that the normal range of a human body temperature varies depending on factors such as the rate of metabolism, the particular organ and other factors. For purposes herein, physiological temperature is the temperature that exists for a non-fasting, comfortably dressed subject that is indoors in a room that is kept at a normal room temperature (e.g. 22.7 to 24.4° C.).

As used herein, reversible refers to a modified enzyme whose activity at the permissive temperature is capable of being recovered or partially recovered upon exposure to the nonpermissive temperature and reexposure to the permissive temperature. Hence, the activity of a reversible enzyme once it is exposed to the nonpermissive temperature is the same or substantially retained compared to the activity of the enzyme exposed only to the permissive conditions and is greater then the activity of the enzyme exposed only to the nonpermissive temperature. For example, upon return to permissive conditions from nonpermissive conditions, reversible enzymes exhibit at or about 120%, 125%, 130%, 140%, 150%, 160%, 170%, 180%, 200% or more the activity of the enzyme exposed only to the nonpermissive temperatures and retain the activity of the enzyme exposed only to the permissive temperature.

As used herein, irreversible or nonreversible refers to a modified enzyme whose enzymatic activity at the permissive temperature is not recovered upon exposure to the nonpermissive temperature and reexposure to the permissive temperature. Hence, the activity of an irreversible enzyme once it is exposed to the nonpermissive temperature is less then the activity of the enzyme exposed only to the permissive temperature and also is less then or the same or substantially the same as the activity of the enzyme exposed only to the nonpermissive conditions. For example, upon return to permissive conditions, irreversible enzymes exhibit at or about 20%, 30%, 40%, 50%, 60%, 70%, 80%, 90%, 100%, 105%, 110%, 115%, or 120% the activity at nonpermissive temperatures and less then 100% of the activity at the activity of the enzyme exposed only to the permissive temperature.

As used herein, a domain refers to a portion (a sequence of three or more, generally 5 or 7 or more amino acids) of a polypeptide that is a structurally and/or functionally distinguishable or definable. For example, a domain includes those that can form an independently folded structure within a protein made up of one or more structural motifs (e.g. combinations of alpha helices and/or beta strands connected by loop regions) and/or that is recognized by virtue of a functional activity, such as kinase activity. A protein can have one, or more than one, distinct domain. For example, a domain can be identified, defined or distinguished by homology of the sequence therein to related family members, such as homology and motifs that define an extracellular domain. In another example, a domain can be distinguished by its function, such as by enzymatic activity, e.g. kinase activity, or an ability to interact with a biomolecule, such as DNA binding, ligand binding, and dimerization. A domain independently can exhibit a function or activity such that the domain independently or fused to another molecule can perform an activity, such as, for example proteolytic activity or ligand binding. A domain can be a linear sequence of amino acids or a non-linear sequence of amino acids from the polypeptide. Many polypeptides contain a plurality of domains. For example, the domain structure of MMPs is set forth in FIG. 1. Those of skill in the art are familiar with domains and can identify them by virtue of structural and/or functional homology with other such domains.

As used herein, a catalytic domain refers to any part of a polypeptide that exhibits a catalytic or enzymatic function. Such domains or regions typically interact with a substrate to result in catalyis thereof. For MMPs, the catalytic domain contains a zinc binding motif, which contains the Zn²⁺ ion bound by three histidine residues and is represented by the conserved sequence HExxHxxGxxH.

As used herein, a proline rich linker (also called the hinge region) refers to a flexible hinge or linker region that has no determinable function. Such a region is typically is found between domains or regions and contributes to the flexibility of a polypeptide.

As used herein, a hemopexin binding domain or haemopexin-like C-terminal domain refers to the C-terminal region of MMP. It is a four bladed β-propeller structure, which is involved in protein-protein interactions. For example, the hemopexin binding domain of MMPs interact with various substrates and also interact with inhibitors, for example, tissue inhibitor of metalloproteases (TIMPs).

As used herein, consisting essentially of or recitation that a polypeptide consists essentially of a particular domain, for example the catalytic domain means that the only MMP portion of the polypeptide is the domain or a catalytically active portion thereof. The polypeptide optionally can include additional non-MMP-derived sequences of amino acids, typically at least 3, 4, 5, 6 or more, such as by insertion into another polypeptide or linkage thereto.

As used herein, a “zymogen” refers to an enzyme that is an inactive precursor of and requires some change, such as chemical modification or proteolysis of the polypeptide, to become active. Some zymogens also require the addition of co-factors such as, but not limited to, pH, ionic strength, metal ions, reducing agents, or temperature for activation. Zymogens include the proenzyme form of enzymes. Hence, zymogens, generally, are inactive and can be converted to a mature polypeptide by chemical modification or catalytic or autocatalytic cleavage of the proregion from the zymogen in the presence or absence of additional cofactors.

As used herein, a pro segment or proregion or propeptide refers to a region or a segment that is cleaved to produce a mature protein. A propeptide is a sequence of amino acids positioned at the amino terminus of a mature polypeptide and can be as little as a few amino acids or can be a multidomain structure. This can include segments that function to suppress the enzymatic activity by masking the catalytic machinery. Propeptides also can act to maintain the stability of an enzyme.

As used herein, a “processing agent” refers to an agent that activates a MMP by facilitating removal of the propeptide or proregion from the zymogen or inactive form of the enzyme. A processing agent includes chemical agents, proteases and other agents such as acidic pH or heat. Exemplary processing agents include, but are not limited to, trypsin, furin, or 4-aminophenylmercuric acetate (AMPA). Other exemplary processing agents are listed in Table 4.

As used herein, a “catalytically active fragment” refers to a polypeptide fragment that contains the catalytically active domain of the enzyme. It is understood that reference to a catalytically active fragment does not necessarily mean that the fragmnent exhibits activity, but only that is contains the catalytically active domain or portion thereof that is required for activity. Hence, a catalytically active fragment is the portion that, under appropriate conditions (e.g. permissive temperature), can exhibit catalytic activity. For example, a catalytically active fragment of a tsMMP-1 (containing at least one mutation that confers a temperature sensitive phenotype) exhibits activity when it is provided at the requisite permissive temperature (e.g. 18° C. to 25° C.), but exhibits substantially reduced or no activity at the non-permissive temperature (e.g. physiological temperature of the body).

As used herein, an active enzyme refers to an enzyme that exhibits enzymatic activity. For purposes herein, active enzymes are those that cleave any one or more components of the ECM, such as collagen. Active enzymes include those that are processed from the zymogen form into the mature form.

As used herein, reference to the “mature” form or “processed mature” form of an enzyme refers to enzymes that do not include the prosegment or proregion of the enzyme. It can be produced from the zymogen or pro-enzyme by activation cleavage in which a prosegment or proregion of the proenzyme is processed to produce the mature form. Hence, a processed mature enzyme lacks the sequence of amino acids that correspond to the prosegment or proregion. It is understood that reference to a processed mature form of an enzyme includes synthetic sequences, and thus does not necessarily require that the enzyme actually is processed to remove the prosegment or proregion. It is understood that any MMP enzyme that lacks the prosegment or proregion sequence is a mature enzyme. For example, SEQ ID NO:709 is the mature sequence of MMP-1. The processed mature form of an enzyme can exhibit activity, and is thus an active enzyme, under appropriate conditions. For example, under physiological conditions, the mature form of MMP-1 is an active enzyme. In contrast, tsMMP-1 variants provided herein exhibit enzymatic activity at the permissive temperature of 18° C. to 25° C. and substantially reduced or no activity at the physiological temperature of the body.

As used herein, an activating condition refers to any physical condition or combination of conditions that is required for an enzyme's activity. For purposes herein, an activating condition for an activatable matrix-degrading enzyme (AMDE), for example, a matrix metalloprotease (MMP) includes those that are not present at the site of administration, for example, not present in the extracellular matrix, in amounts (i.e. quantity, degree, level or other physical measure) required for activation of the enzyme. Exemplary of activating conditions include temperature. For example, in the case of the interstitium, the physiological temperature is at or about 37° C. An activating condition is a temperature that is not at or about 37° C., but that is cooler or warmer. By virtue of the fact that the activating condition is not present at the site of administration of the enzyme, but must be added exogenously, the activating condition will dissipate over time as the temperature adjusts, such that the activating condition is no longer present to activate the enzyme. Hence, the enzyme will be active for a limited or predetermined time upon administration.

As used herein, an activator refers to any composition or other material or item that provides an activating condition for an activatable matrix-degrading enzyme. For purposes herein, an activator refers to any item that is capable of providing a temperature condition at the permissive temperature of the enzyme. Examples of activators include, but are not limited to hot or cold buffers or hot or cold packs.

As used herein, an “activatable matrix-degrading enzyme (AMDE)” refers to a matrix degrading enzyme that requires an activating condition in order to be active. For purposes herein, for example, an AMDE is substantially inactive in the ECM unless exposed to activators before, with or subsequent to administration of the AMDE, thereby providing an activating condition for the enzyme. Hence, activation of a activatable enzymes is controlled by exogenous conditions so that the period of time at an in vivo locus or site during which the enzyme is active can be predetermined and/or controlled as a result of the dissipation and/or neutralization of the activation condition (i.e. temporally controllable or time-controlled). Thus, by virtue of exposure to an activating condition, the enzymes are active for a limited time and/or to a limited extent in the ECM (i.e. are conditionally active). The extent and time of activation can be controlled by selection of activator or activating conditions, and can be for a predetermined time. For example, temperature sensitive enzyme, such as a tsMMP, is activatable in that it can be activated by exposure to the activating condition of temperature, such as provided by a cold buffer or other liquid solution. Upon administration of the activated enzyme with the activator to the physiologic temperature environment of the ECM, the temperature will adjust to and eventually return to the physiologic temperature in a time period that can be predetermined based upon the initial temperature of the activator, the site of administration, the depth of administration and other factors, such that the enzyme will become inactive or less active.

As used herein, a “therapeutically effective amount” or a “therapeutically effective dose” refers to an agent, compound, material, or composition containing a compound that is at least sufficient to produce a therapeutic effect.

As used herein, an enzyme that is active for a limited time or for limited duration refers to an active enzyme having activity that dissipates and/or is neutralized over time. Thus, by virtue of the absence of an activation condition, the enzyme is rendered inactive.

As used herein, predetermined time means a limited time that is known before and can be controlled. The dissipation and/or neutralization of an activation condition required for an enzyme's activity can be titrated or otherwise empirically determined so that the time required for an active enzyme to become inactive is known. For purposes herein, for example, an enzyme can be active for a predetermined time that is or is about 1 minutes, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hour, 3 hour, or 4 hour. The predetermined time can be controlled by the subject or the treating physician, for example, where a cold pack or hot pack is used as the activator. Further, it is understood that reversible enzymes can be re-activated by exposure to permissive conditions, and thereby can be active for an additional predetermined time.

As used herein, sub-epidermal administration refers to any administration that results in delivery of the enzyme under the outer-most layer of the skin. Sub-epidermal administration does not include topical application onto the outer layer of the skin. Examples of sub-epidermal administrations include, but are not limited to, subcutaneous, intramuscular intralesional and intradermal routes of administration.

As used herein, substrate refers to a molecule that is cleaved by an enzyme. Minimally, a target substrate includes a peptide containing the cleavage sequence recognized by the protease, and therefore can be two, three, four, five, six or more residues in length. A substrate also includes a full-length protein, allelic variant, isoform or any portion thereof that is cleaved by an enzyme. Additionally, a substrate includes a peptide or protein containing an additional moiety that does not affect cleavage of the substrate by the enzyme. For example, a substrate can include a four amino acid peptide, or a full-length protein chemically linked to a fluorogenic moiety.

As used herein, cleavage refers to the breaking of peptide bonds or other bonds by an enzyme that results in one or more degradation products.

As used herein, activity refers to a functional activity or activities of a polypeptide or portion thereof associated with a full-length (complete) protein. Functional activities include, but are not limited to, biological activity, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide.

As used herein, enzymatic activity or catalytic activity or cleavage activity refers to the activity of a protease as assessed in in vitro proteolytic assays that detect proteolysis of a selected substrate.

As used herein, an inactive enzyme refers to an enzyme that exhibits substantially no activity (i.e. catalytic activity or cleavage activity), such as less than 10% of the maximum activity of the enzyme. The enzyme can be inactive by virtue of its conformation, the absence of an activating conditions required for its activity, or the presence of an inhibitor or any other condition or factor or form that renders the enzyme substantially inactive.

As used herein, “retains an activity” refers to the activity exhibited by a modified MMP polypeptide at a particular condition compared to at another condition or to another polypeptide. For example, it is the activity a modified MMP polypeptide exhibits as compared to an unmodified MMP polypeptide of the same form and under the same conditions. It also can be the activity a modified MMP polypeptide exhibits as compared to the modified MMP polypeptide under different conditions, for example, different temperature conditions. Generally, a modified MMP polypeptide that retains an activity exhibits increased or decreased activity compared to an unmodified polypeptide under the same conditions or compared to the unmodified polypeptide under different conditions. For example, the modified MMP polypeptide can retain 40%, 50%, 60%, 70%, 80%, 90%, 100%, 110%, 120%, 130%, 140%, 150%, 160%, 170%, 180%, 190%, 200%, 300%, 400%, 500% or more of the enzymatic activity.

As used herein, a human protein is one encoded by a nucleic acid molecule, such as DNA, present in the genome of a human, including all allelic variants and conservative variations thereof. A variant or modification of a protein is a human protein if the modification is based on the wildtype or prominent sequence of a human protein.

As used herein, hyaluronidase refers to an enzyme that degrades hyaluronic acid. Hyaluronidases include bacterial hyaluronidases (EC 4.2.99.1), hyaluronidases from leeches, other parasites, and crustaceans (EC 3.2.1.36), and mammalian-type hyaluronidases (EC 3.2.1.35). Hyaluronidases also include any of non-human origin including, but not limited to, murine, canine, feline, leporine, avian, bovine, ovine, porcine, equine, piscine, ranine, bacterial, and any from leeches, other parasites, and crustaceans. Exemplary non-human hyaluronidases include any set forth in any of SEQ ID NOS: 3482-3505. Exemplary human hyaluronidases include HYAL1 (SEQ ID NO:3469), HYAL2 (SEQ ID NO:3470), HYAL3 (SEQ ID NO:3471), HYAL4 (SEQ ID NO:3472), and PH20 (SEQ ID NO:3473). Also included amongst hyaluronidases are soluble human PH20 and soluble rHuPH20.

Reference to hyaluronidases includes precursor hyaluronidase polypeptides and mature hyaluronidase polypeptides (such as those in which a signal sequence has been removed), truncated forms thereof that have activity, and includes allelic variants and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the precursor polypeptide set forth any of SEQ ID NO: 3473 or the mature form thereof. Hyaluronidases also include those that contain chemical or posttranslational modifications and those that do not contain chemical or posttranslational modifications. Such modifications include, but are not limited to, PEGylation, albumination, glycosylation, farnesylation, carboxylation, hydroxylation, phosphorylation, and other polypeptide modifications known in the art.

As used herein, soluble human PH20 or sHuPH20 include mature polypeptides lacking all or a portion of the glycosylphospatidylinositol (GPI) attachment site at the C-terminus such that upon expression, the polypeptides are soluble. Exemplary sHuPH20 polypeptides include mature polypeptides having an amino acid sequence set forth in any one of SEQ ID NOS:3476-3481. The precursor polypeptides for such exemplary sHuPH20 polypeptides include an amino acid signal sequence. Exemplary of a precursor is set forth in SEQ ID NO:3473, which contains a 35 amino acid signal sequence at amino acid positions 1-35. Soluble HuPH20 polypeptides can be degraded during or after the production and purification methods described herein.

As used herein, soluble rHuPH20 refers to a soluble form of human PH20 that is recombinantly expressed in Chinese Hamster Ovary (CHO) cells. Soluble rHuPH20 is encoded by nucleic acid that includes the signal sequence and is set forth in SEQ ID NO:3475. Also included are DNA molecules that are allelic variants thereof and other soluble variants. The nucleic acid encoding soluble rHuPH20 is expressed in CHO cells which secrete the mature polypeptide. As produced in the culture medium there is heterogeneity at the C-terminus so that the product includes a mixture of species of SEQ ID NOS:3476-3481. Corresponding allelic variants and other variants also are included. Other variants can have 60%, 70%, 80%, 85%, 90%, 91%, 92%, 93%, 94%, 95%, 96%, 97%, 98%, 99% or more sequence identity with any of SEQ ID NOS:3476-3481 as long they retain a hyaluronidase activity and are soluble.

As used herein, hyaluronidase activity refers to any activity exhibited by a hyaluronidase polypeptide. Such activities can be tested in vitro and/or in vivo and include, but are not limited to, enzymatic activity, such as to effect cleavage of hyaluronic acid, ability to act as a dispersing or spreading agent and antigenicity.

As used herein, the residues of naturally occurring α-amino acids are the residues of those 20 α-amino acids found in nature which are incorporated into protein by the specific recognition of the charged tRNA molecule with its cognate mRNA codon in humans.

As used herein, nucleic acids include DNA, RNA and analogs thereof, including peptide nucleic acids (PNA) and mixtures thereof. Nucleic acids can be single or double-stranded. When referring to probes or primers, which are optionally labeled, such as with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally a probe or primer contains at least 14, 16 or 30 contiguous nucleotides of sequence complementary to or identical to a gene of interest. Probes and primers can be 10, 20, 30, 50, 100 or more nucleic acids long.

As used herein, a peptide refers to a polypeptide that is from 2 to 40 amino acids in length.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain).

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243: 3552-3559 (1969), and adopted 37 C.F.R. §§1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | proline |
| K | Lys | Lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | asparagine |
| B | Asx | Asn and/or Asp |
| C | Cys | Cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence (Table 1) and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues, to an amino-terminal group such as NH₂ or to a carboxyl-terminal group such as COOH.

As used herein, “naturally occurring amino acids” refer to the 20 L-amino acids that occur in polypeptides.

As used herein, “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally-occurring amino acids and include, but are not limited to, the D-isostereomers of amino acids. Exemplary non-natural amino acids are described herein and are known to those of skill in the art.

As used herein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Such substitutions can be made in accordance with those set forth in TABLE 2 as follows:

TABLE 2

| Original residue | Exemplary conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (B) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, a DNA construct is a single or double stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule can not be paired. Such unpaired ends will, in general, not exceed 20 nucleotides in length.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

“Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exists a number of methods to measure identity between two polynucleotide or polypeptides, the term “identity” is well known to skilled artisans (Carillo, H. & Lipton, D., SIAM J Applied Math 48:1073 (1988)).

As used herein, homologous (with respect to nucleic acid and/or amino acid sequences) means about greater than or equal to 25% sequence homology, typically greater than or equal to 25%, 40%, 50%, 60%, 70%, 80%, 85%, 90% or 95% sequence homology; the precise percentage can be specified if necessary. For purposes herein the terms “homology” and “identity” are often used interchangeably, unless otherwise indicated. In general, for determination of the percentage homology or identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence homology, the number of conserved amino acids is determined by standard alignment algorithms programs, and can be used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

Whether any two molecules have nucleotide sequences or amino acid sequences that are at least 60%, 70%, 80%, 85%, 90%, 95%, 96%, 97%, 98% or 99% “identical” or “homologous” can be determined using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA (Atschul, S. F., et al., J Molec Biol 215:403 (1990)); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” or “homology” represents a comparison between a test and a reference polypeptide or polynucleotide. As used herein, the term at least “90% identical to” refers to percent identities from 90 to 99.99 relative to the reference nucleic acid or amino acid sequence of the polypeptide. Identity at a level of 90% or more is indicative of the fact that, assuming for exemplification purposes a test and reference polypeptide length of 100 amino acids are compared, no more than 10% (i.e., 10 out of 100) of the amino acids in the test polypeptide differs from that of the reference polypeptide. Similar comparisons can be made between test and reference polynucleotides. Such differences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often by manual alignment without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “primer” refers to a nucleic acid molecule that can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g. by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application. Complementary, when referring to two nucleotide sequences, means that the two sequences of nucleotides are capable of hybridizing, typically with less than 25%, 15% or 5% mismatches between opposed nucleotides. If necessary, the percentage of complementarity will be specified. Typically the two molecules are selected such that they will hybridize under conditions of high stringency.

As used herein, substantially identical to a product means sufficiently similar so that the property of interest is sufficiently unchanged so that the substantially identical product can be used in place of the product.

As used herein, it also is understood that the terms “substantially identical” or “similar” varies with the context as understood by those skilled in the relevant art.

As used herein, an allelic variant or allelic variation references any of two or more alternative forms of a gene occupying the same chromosomal locus. Allelic variation arises naturally through mutation, and can result in phenotypic polymorphism within populations. Gene mutations can be silent (no change in the encoded polypeptide) or can encode polypeptides having altered amino acid sequence. The term “allelic variant” also is used herein to denote a protein encoded by an allelic variant of a gene. Typically the reference form of the gene encodes a wildtype form and/or predominant form of a polypeptide from a population or single reference member of a species. Typically, allelic variants, which include variants between and among species typically have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% identity or greater with a wildtype and/or predominant form, including 96%, 97%, 98%, 99% or greater identity with a wildtype and/or predominant form of a polypeptide. Reference to an allelic variant herein generally refers to variations n proteins among members of the same species.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include substitutions, deletions and insertions of nucleotides. An allele of a gene also can be a form of a gene containing a mutation.

As used herein, species variants refer to variants in polypeptides among different species, including different mammalian species, such as mouse and human.

As used herein, a splice variant refers to a variant produced by differential processing of a primary transcript of genomic DNA that results in more than one type of mRNA.

As used herein, modification is in reference to modification of a sequence of amino acids of a polypeptide or a sequence of nucleotides in a nucleic acid molecule and includes deletions, insertions, and replacements of amino acids and nucleotides, respectively. Methods of modifying a polypeptide are routine to those of skill in the art, such as by using recombinant DNA methodologies.

As used herein, the term promoter means a portion of a gene containing DNA sequences that provide for the binding of RNA polymerase and initiation of transcription. Promoter sequences are commonly, but not always, found in the 5′ non-coding region of genes.

As used herein, isolated or purified polypeptide or protein or biologically-active portion thereof is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

The term substantially free of cellular material includes preparations of proteins in which the protein is separated from cellular components of the cells from which it is isolated or recombinantly-produced. In one embodiment, the term substantially free of cellular material includes preparations of enzyme proteins having less that about 30% (by dry weight) of non-enzyme proteins (also referred to herein as a contaminating protein), generally less than about 20% of non-enzyme proteins or 10% of non-enzyme proteins or less that about 5% of non-enzyme proteins. When the enzyme protein is recombinantly produced, it also is substantially free of culture medium, i.e., culture medium represents less than about or at 20%, 10% or 5% of the volume of the enzyme protein preparation.

As used herein, the term substantially free of chemical precursors or other chemicals includes preparations of enzyme proteins in which the protein is separated from chemical precursors or other chemicals that are involved in the synthesis of the protein. The term includes preparations of enzyme proteins having less than about 30% (by dry weight) 20%, 10%, 5% or less of chemical precursors or non-enzyme chemicals or components.

As used herein, synthetic, with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by recombinant means by using recombinant DNA methods means the use of the well known methods of molecular biology for expressing proteins encoded by cloned DNA.

As used herein, vector (or plasmid) refers to discrete elements that are used to introduce a heterologous nucleic acid into cells for either expression or replication thereof. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, an expression vector includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, vector also includes “virus vectors” or “viral vectors.” Viral vectors are engineered viruses that are operatively linked to exogenous genes to transfer (as vehicles or shuttles) the exogenous genes into cells.

As used herein, operably or operatively linked when referring to DNA segments means that the segments are arranged so that they function in concert for their intended purposes, e.g., transcription initiates in the promoter and proceeds through the coding segment to the terminator.

As used herein the term assessing is intended to include quantitative and qualitative determination in the sense of obtaining an absolute value for the activity of a protease, or a domain thereof, present in the sample, and also of obtaining an index, ratio, percentage, visual or other value indicative of the level of the activity. Assessment can be direct or indirect and the chemical species actually detected need not of course be the proteolysis product itself but can for example be a derivative thereof or some further substance. For example, detection of a cleavage product of a substrate, such as by SDS-PAGE and protein staining with Coomasie blue.

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities can be observed in in vitro systems designed to test or use such activities. Thus, for purposes herein a biological activity of a protease is its catalytic activity in which a polypeptide is hydrolyzed.

As used herein equivalent, when referring to two sequences of nucleic acids, means that the two sequences in question encode the same sequence of amino acids or equivalent proteins. When equivalent is used in referring to two proteins or peptides, it means that the two proteins or peptides have substantially the same amino acid sequence with only amino acid substitutions that do not substantially alter the activity or function of the protein or peptide. When equivalent refers to a property, the property does not need to be present to the same extent (e.g., two peptides can exhibit different rates of the same type of enzymatic activity), but the activities are usually substantially the same.

As used herein, “modulate” and “modulation” or “alter” refer to a change of an activity of a molecule, such as a protein. Exemplary activities include, but are not limited to, biological activities, such as signal transduction. Modulation can include an increase in the activity (i.e., up-regulation or agonist activity) a decrease in activity (i.e., down-regulation or inhibition) or any other alteration in an activity (such as a change in periodicity, frequency, duration, kinetics or other parameter). Modulation can be context dependent and typically modulation is compared to a designated state, for example, the wildtype protein, the protein in a constitutive state, or the protein as expressed in a designated cell type or condition.

As used herein, a composition refers to any mixture. It can be a solution, suspension, liquid, powder, paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a kit is a packaged combination that optionally includes other elements, such as additional reagents and instructions for use of the combination or elements thereof.

As used herein, “disease or disorder” refers to a pathological condition in an organism resulting from cause or condition including, but not limited to, infections, acquired conditions, genetic conditions, and characterized by identifiable symptoms. Diseases and disorders of interest herein are those involving components of the ECM.

As used herein, an ECM-mediated disease or condition is one where any one or more ECM components is involved in the pathology or etiology. For purposes herein, an ECM-mediated disease or conditions includes those that are caused by an increased deposition or accumulation of one or more ECM component. Such conditions include, but are not limited to, cellulite, Duputyren's syndrome, Peyronie's disease, frozen shoulders, existing scars such as keloids, scleroderma and lymphedema.

As used herein, “treating” a subject with a disease or condition means that the subject's symptoms are partially or totally alleviated, or remain static following treatment. Hence treatment encompasses prophylaxis, therapy and/or cure. Prophylaxis refers to prevention of a potential disease and/or a prevention of worsening of symptoms or progression of a disease. Treatment also encompasses any pharmaceutical use of a modified interferon and compositions provided herein.

As used herein, a pharmaceutically effective agent, includes any therapeutic agent or bioactive agents, including, but not limited to, for example, anesthetics, vasoconstrictors, dispersing agents, conventional therapeutic drugs, including small molecule drugs and therapeutic proteins.

As used herein, treatment means any manner in which the symptoms of a condition, disorder or disease or other indication thereof is/are ameliorated or otherwise beneficially altered.

As used herein therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject. A therapeutically effective amount effects treatment.

As used herein, the term “subject” refers to an animal, including a mammal, such as a human being.

As used herein, a patient refers to a human subject.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, prevention or prophylaxis refers to methods in which the risk of developing disease or condition is reduced.

As used herein, an effective amount is the quantity of a therapeutic agent necessary for preventing, curing, ameliorating, arresting or partially arresting a symptom of a disease or disorder.

As used herein, unit dose form refers to physically discrete units suitable for human and animal subjects and packaged individually as is known in the art.

As used herein, a single dosage formulation refers to a formulation for direct administration.

As used herein, an “article of manufacture” is a product that is made and sold. As used throughout this application, the term is intended to encompass activatable matrix degrading enzymes contained in articles of packaging.

As used herein, fluid refers to any composition that can flow. Fluids thus encompass compositions that are in the form of semi-solids, pastes, solutions, aqueous mixtures, gels, lotions, creams and other such compositions.

As used herein, a “kit” refers to a combination of an activatable matrix-degrading enzyme provided herein and another item for a purpose including, but not limited to, activation, administration, diagnosis, and assessment of a biological activity or property. Kits optionally include instructions for use.

As used herein, a cellular extract or lysate refers to a preparation or fraction which is made from a lysed or disrupted cell.

As used herein, animal includes any animal, such as, but are not limited to primates including humans, gorillas and monkeys; rodents, such as mice and rats; fowl, such as chickens; ruminants, such as goats, cows, deer, sheep; ovine, such as pigs and other animals. Non-human animals exclude humans as the contemplated animal. The enzymes provided herein are from any source, animal, plant, prokaryotic and fungal. Most enzymes are of animal origin, including mammalian origin.

As used herein, a control refers to a sample that is substantially identical to the test sample, except that it is not treated with a test parameter, or, if it is a plasma sample, it can be from a normal volunteer not affected with the condition of interest. A control also can be an internal control.

As used herein, the singular forms “a,” “an” and “the” include plural referents unless the context clearly dictates otherwise. Thus, for example, reference to a compound, comprising “an extracellular domain” includes compounds with one or a plurality of extracellular domains.

As used herein, ranges and amounts can be expressed as “about” a particular value or range. About also includes the exact amount. Hence “about 5 bases” means “about 5 bases” and also “5 bases.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

### B. OVERVIEW—TEMPERATURE SENSITIVE MATRIX METALLOPROTEASES AND OTHER MODIFIED METALLOPROTEASES

Provided herein are modified MMP polypeptides, for example temperature sensitive (ts) mutants of matrix metalloproteases (tsMMPs), that degrade one or more components of the extracellular matrix (ECM). The tsMMPs can degrade one or more components of the ECM in a temperature-dependent manner. In particular, mutants provided herein degrade a collagen. In some examples, the mutants display higher activity at lower temperatures (e.g. 25° C.) then at higher temperatures, for example, physiologic temperatures (e.g. 37° C.). In other examples, the mutants display higher activity at physiologic temperatures then at lower temperatures. Thus, the activation of the tsMMPs, for example upon administration to the body, can be temporally and conditionally controlled by virtue of changes in temperature.

Uncontrolled MMP activity can be highly disruptive to tissue integrity. By virtue of the conditional activation of activatable tsMMPs, temporary activation is achieved, thereby regulating the duration of enzymatic action on extracellular matrix (ECM) components to reduce deleterious side effects associated with unwanted prolonged activation of enzymes. This is an advantage of the present tsMMPs over existing collagenase treatments. Hence, an advantage of such mutants is that their activity can be regulated, thereby permitting the use of tsMMPs to treat diseases and/or conditions of the ECM.

Modified MMP polypeptides provided herein are modified to exhibit temperature sensitivity via increased activity at a permissive temperature compared to a nonpermissive temperature and/or are modified as activity mutants to exhibit increased activity compared to the MMP polypeptide not containing the modification. The modified MMP polypeptides provided herein are modified, for example, by amino acid substitution, insertion or replacement. For example, tsMMPs contain one or more amino acid replacements in their primary sequence rendering the protein more active at permissive temperatures then at non-permissive temperatures. Modified MMP polypeptides provided herein can contain 1, 2, 3, 4, 5, 6, 7, 8, 9, 10, 11, 12, 13, 14, 15, 16, 17, 18, 19, 20 or more amino acid modifications. In particular, modified MMP polypeptides, for example tsMMPs, provided herein contain 1, 2, 3, 4, 5, 6, 7, 8, 9 or 10 amino acids modifications.

tsMMPs provided herein are activatable at a permissive temperature, but are less active or inactive at other non-permissive temperatures. The tsMMPs provided herein have a ratio of activity at a permissive temperature compared to a non-permissive temperature that is or is about 1.1, 1.2, 1.3, 1.4, 1.5, 1.6, 1.7, 1.8, 1.9, 2.0, 2.5, 3.0, 3.5, 4.0, 4.5, 5, 5.5, 6, 6.5, 7, 7.5, 8, 8.5, 9, 9.5, 10, 15, 20, 30, 40, 50 or more. Thus, the activity of the tsMMPs provided herein at the non-permissive temperature is or is about 70%, 65%, 60%, 55%, 50%, 45%, 40%, 35%, 30%, 25%, 20%, 15%, 10%, 5%, 4%, 3%, 2%, 1%, 0.5% or less of the activity at a permissive temperature.

For example, MMPs that are normally active at physiological temperature (e.g. 37° C.) are modified and enzymes selected that are active at lower temperatures, i.e. temperatures below the physiological temperature of the body (e.g. less than 37° C.; e.g. at or about 20° C., 21° C., 22° C., 230C, 240C, 250C, 260C, 270C, 28° C., 290C or 30° C.), but that are less active or inactive at physiologic temperature. Such modified enzymes can be used as activatable matrix-degrading enzymes (AMDE) where the activation condition is low temperature. The activation of the enzyme is temporally controlled as the in vivo temperature returns to the physiological temperature of 37° C. Thus, for example, tsMMPs provided herein are active at a permissive temperature that is at or about 25° C., but are less active at higher temperatures such as at or about 330C, 340C, 35° C., 360C, 370C, 38° C. or 39° C. The tsMMPs provided herein have a ratio of activity at the permissive temperature of at or about 25° C. compared to a non-permissive temperature of at or about 34° C. or 37° C., for example, 33° C., 34° C., 35° C., 36° C., 37° C., 38° C. or 39° C., that is or is about 1.1, 1.2, 1.3, 1.4, 1.5, 1.6, 1.7, 1.8, 1.9, 2.0, 2.5, 3.0, 3.5, 4.0, 4.5, 5, 5.5, 6, 6.5, 7, 7.5, 8, 8.5, 9, 9.5, 10, 15, 20, 30, 40, 50 or more. Thus, the activity of the tsMMPs provided herein at the non-permissive temperature of at or about 34° C. or 37° C. is or is about 70%, 65%, 60%, 55%, 50%, 45%, 40%, 35%, 30%, 25%, 20%, 15%, 10%, 5%, 4%, 3%, 2%, 1%, 0.5% or less of the activity at the permissive temperature at or about 25° C.

For example, modified MMPs polypeptides provided herein, in particular modified MMP-1 polypeptides, that exhibit temperature sensitivity are conditionally active and can be used in uses, methods and processes of treating ECM-mediated diseases and disorders. For example, such tsMMP polypeptides are active at a permissive temperature that is below the normal temperature of the ECM. Thus, when administered to the ECM at or below the permissive temperature, the enzymes exhibit activity. In one example, before administration, a tsMMP, for example tsMMP-1, can be reconstituted in a cold buffer and/or can be stored at a cold temperature that that is at or below the permissive temperature. The tsMMP exhibits activity when exposed to the permissive temperature (e.g. 18° C. to 25° C.). As the tsMMP is exposed to a steadily warmer temperature approaching or reaching the nonpermissive temperature, for example upon administration to the body due to the physiologic temperature of the body, the activity of the MMP is reduced. Thus, the tsMMP exhibits conditional activity, conditioned upon maintenance of a permissive temperature. For example, the activity of the ECM can be controlled for a predetermined time by maintaining the ECM below the physiological temperature of the body.

Thus, where the activating condition is temperature, an activator can be provided that exposes the tsMMP to the permissive temperature required for activation. The exposure to the activator can be in vitro or in vivo. The activator can be exposed to the tsMMP prior to, simultaneously, subsequently or intermittently upon in vivo administration. The activator can provide the requisite heat or cold required for activation. For example, where the activating condition is low temperature, the activator can be provided as a cold buffer or as an ice pack to be applied to the site of administration. Where the activating condition is heat, the activator can be provided as a warm buffer or as a heat pack to be applied to the site of administration. The activating condition also can be provided by storage of the tsMMP at the permissive temperature immediately and just prior to use. The duration of exposure to the activator can be continuous, can be for a predetermined time, or can be intermittent (for example, if the tsMMP is reversible). Thus, the time period permitting activation is flexible and can be adapted to the particular enzyme that is used, the disease or condition being treated, the site of administration or other factors. It is within the level of the skilled artisan to determine the duration of exposure to the activator.

In the absence of exposure to the activator providing the activating condition, the tsMMPs present at the non-permissive temperature are inactive or substantially inactive compared to the activity at the permissive temperature. The activating condition of a permissive temperature (e.g. low temperature) not normally present at the site of administration permits the temporal regulation of, and alteration of, the physiological parameters of organs and tissues, such as the interstitium that exhibits a physiologic temperature of approximately 37° C. Under normal physiological conditions, the temperature of the interstitium is approximately 37° C. Thus, for example, tsMMPs active at low temperatures, when present in the interstitium would normally be catalytically inactive because of the physiologic temperature of the interstitium. When the temperature of the interstitium is temporarily rendered cold, for example, by exposure to a cold buffer or to a cold pack administered on the adjacent surface, tsMMPs when administered to the interstitium will become activated. When the temperature increases and returns to physiological levels, then the tsMMPs become inactive or substantially inactive and cease to exert their enzymatic activity. Hence, by taking advantage of the requirement for exogenous activating conditions, tsMMPs are activatable and can be made temporally active for a limited duration during use, such as upon in vivo administration to the body.

The tsMMPs provided herein include those that are irreversibly inactive following exposure to non-permissive temperatures. Such mutants are active when exposed to permissive temperature conditions (e.g. 25° C.), but are less active or inactive when the temperature is altered to a non-permissive temperatures (e.g. 37° C., such as can occur upon in vivo administration to the body and removal of an exogenous activator (e.g. cold pack)). Upon return to permissive conditions, irreversible tsMMP polypeptides provided herein exhibit at or about 50%, 60%, 70%, 80%, 90%, 100%, 105%, 110%, 115%, or 120% the activity at non-permissive temperatures. The activity is not reversible.

Also provided herein are tsMMPs that are reversibly inactive following exposure to a non-permissive temperature. Such mutants are active when exposed to a permissive temperature condition, but are less active or inactive when the temperature is altered to a non-permissive temperatures. Upon renewed exposure to an activating condition providing the permissive temperature (e.g. cold pack), the activity of the tsMMP is restored, thereby rendering the enzyme sufficiently active to degrade one or more components of the ECM. For example, upon return to permissive conditions from nonpermissive conditions, reversible tsMMP polypeptides provided herein exhibit at or about 120%, 125%, 130%, 140%, 150%, 160%, 170%, 180%, 200% or more the activity at non-permissive temperatures.

tsMMPs provided herein retain one or more activities of wildtype MMP, for example, enzymatic activity for cleavage of an ECM component such as collagen. For example, a tsMMP provided herein retains an activity at the permissive temperature that is or is about 30%, 40%, 50%, 60%, 70%, 80%, 90%, 100%, 110%, 120%, 140%, 150% or more the activity of wildtype MMP at the permissive temperature. Thus, tsMMPs provided herein include those that are more active than wildtype MMP-1 at the permissive temperature, and also those that are less active than wildtype MMP-1 at the permissive temperature. Generally, tsMMPs provided herein, however, are less active then wildtype MMP-1 at the nonpermissive temperature. For example, tsMMPs provided herein exhibit 95%, 90%, 80%, 70%, 65%, 60%, 55%, 50%, 45%, 40%, 35%, 30%, 25%, 20%, 15%, 10%, 5%, 4%, 3%, 2%, 1%, 0.5%, generally 40%, 30%, 25%, 20%, 15%, 10%, or 5% residual activity of wildtype MMP-1 at physiologic temperature (e.g. 34 or 37° C.).

Typically, modified MMP polypeptides, for example tsMMPs, provided herein are zymogens (containing a propeptide) or processed enzymes (e.g. mature enzymes, lacking a propeptide), or catalytically active forms thereof. As discussed below, most enzymes, including MMPs, are zymogens and require an initial processing event for activity by removal of a propeptide segment from the N-terminal end of the polypeptide. A processing agent, such as a protease or chemical agent, directly or indirectly initiates one or more cleavage events to generate an active MMP by virtue of removal of the propeptide segment and/or conformational changes that expose the active site of the MMP. Hence, normally, upon processing of an enzyme to a mature form, the enzyme is active. The activity of a processed enzyme is not reversible, thereby leading to uncontrolled degradation of the ECM upon administration of the processed enzyme to the body. It is contemplated herein that modification of the enzyme to additionally confer temperature sensitivity provides a mechanism to conditionally and temporally control activation of the MMP to avoid continued activation of the processed MMP.

Any MMP, whether synthetic or isolated from natural sources, such as those set forth in Table 5 or elsewhere herein, mature forms thereof lacking the propeptide, and catalytically active forms including polypeptides containing only the catalytically active domain or a portion thereof, and allelic or species variants or other variants thereof, or any known to those of skill in the art can be modified as described herein to be temperature sensitive and/or have increased activity and is intended for use in the compositions, combinations, methods and apparatus provided herein. It is understood that any modified enzyme form provided herein exhibits increased activity and/or temperature sensitivity, i.e. the enzyme is activatable due to the requirement of a temperature activating condition. Exemplary MMPs that can be modified, for example to be temperature sensitive, are set forth in Table 1 and include, for example, any of SEQ ID NOS: 1, 711, 714, 717, 720, 723, 726, 729, 732, 735, 738, 741, 744, 747, 750, 753, 756, 759, 762, 765, 768, 771, 774 or 777, zymogen forms or mature forms thereof, catalytically active forms thereof, and allelic or species variants or other variants thereof, so long as the other forms contain the mutation conferring temperature sensitivity and/or increased activity. For example, SEQ ID NO:2 is the zymogen form of SEQ ID NO: 1. FIG. 1 exemplifies the zymogen form of other exemplary MMPs. One of skill in the art knows or could identify tsMMPs. For example, one of skill in the art could use routine molecular biology techniques to introduce amino acid mutation(s) herein into an MMP, and test each for enzyme activation under temperature permissive and non-permissive temperatures to assess the requirement of an exogenous activating condition for sustained or reversible activation of any desired enzyme. Exemplary assays for enzyme activation are provided herein and known in the art.

Hence, modified MMP polypeptides, for example tsMMPs, provided herein include zymogen forms (e.g. proenzyme), processed mature forms lacking a propeptide, and polypeptides containing only the catalytically active domains thereof. For example, tsMMPs include zymogen forms (e.g. proenzyme), processed mature forms lacking a propeptide, and polypeptides containing only the catalytically active domains thereof, so long as the tsMMPs exhibits enzymatic activity at the permissive temperature. Exemplary of such a tsMMP is a tsMMP-1. tsMMP-1 provided herein contains one or more amino acid modifications in its primary sequence corresponding to amino acid replacements in a wildtype MMP-1 set forth in SEQ ID NO:2. Exemplary modifications are described elsewhere herein in Section D. The modified MMPs, for example tsMMP-1 mutants or activity mutants, provided herein include those that are zymogens or those that are in a mature form lacking a propeptide. The zymogen or mature polypeptides provided herein include those that are full-length, include all or a portion of the proline rich linker or the hemopexin binding domain, lack all or a portion of the proline rich linker or the hemopexin binding domain, or polypeptides that include only the catalytically active domains thereof (e.g. corresponding to amino acids 81-242 of the sequence of amino acids set forth in SEQ ID NO: 1) so long as the tsMMP-1 retains enzymatic activity at the permissive temperature and/or exhibits increased activity.

It is understood that when provided in zymogen form, the modified MMP polypeptides, for example tsMMPs, are inactive and that processing by a processing agent is required for activity. Generally, the processing of the enzyme is effected prior to use, such as prior to administration in vivo. For example, the processing agent can be applied simultaneously, intermittently or subsequently to exposure of the tsMMP to the activating condition (e.g. low temperature) and administration to the body. Generally, the processing agent is chosen that is acceptable for administration to a subject. If desired, the processing agent can be dialyzed or otherwise purified away from the enzyme preparation before administrations. Thus, for zymogen forms of the enzyme, two steps are required for activation: 1) exposure to a processing agent; and 2) exposure to an activating condition. Whether in zymogen or processed form, exposure of the tsMMP to an activator at the permissive temperature temporally controls activity of a tsMMP.

Modified MMP polypeptides, for example tsMMPs, provided herein can be further modified to alter any one or more properties or activities. For example, altered properties or activities include, but are not limited to, modification that render the enzyme more stable, alter the substrate specificity and/or increase resistance to one or more inhibitors. In one example, modified MMP polypeptides, for example tsMMPs, can be modified to alter its substrate specificity. For example, an enzyme can be modified to have increased specificity for a particular substrate. Thus, for example, a modified MMP polypeptide, which exhibits substrate specificity for type I and type IV collagen can be modified so that it has increased substrate specificity for type I collagen, and not type IV collagen, and vice versa. If desired, enzyme stability also can be increased by PEGylation or glycosylation of the enzyme.

Modifications of polypeptides can be achieved by routine molecular biology techniques, and are within the skill of one in the art. For purposes herein, modified MMP polypeptides, for example tsMMPs, retain one or more activities of the wildtype MMP at the permissive temperature. Retained activity can be 40%, 50%, 60%, 70%, 80%, 90%, 95% or more activity of the wildtype MMP at the permissive temperature. Modified enzymes can be tested for their substrate specificity using routine assays for substrate cleavage such as is described herein, or known in the art. For example, substrate cleavage can be assessed on fluorogenic peptides or on purified proteins. Cleavage can be assessed using in vitro or in vivo assays. For example, cleavage can be assessed by incubating the enzyme with the substrate, and then running the mixture on an SDS-PAGE gel. Degradation can be assessed by Western Blot or by using standard protein stains such as Coomasie Blue or Silver Stain reagents.

The modified MMP polypeptides, for example tsMMPs, are provided herein as compositions, combinations and containers. The modified MMPs, for example tsMMP, are provided in a therapeutically effective amount, that when activated, degrade one or more components of the ECM upon administration, such as upon sub-epidermal administration. The resulting modified MMPs, for example tsMMPs, can be used as therapeutics to treat ECM-mediated diseases or conditions. A description of compositions, combinations, containers and methods of using activatable matrix-degrading proteins is provided in related U.S. Provisional Application Nos. 61/068,667 and 61/127,725, U.S. patent application Ser. No. 12/81,063 and International PCT Application No. PCT/US2009/001489, each incorporated by reference in their entirety. Such description of the compositions, combinations, containers and methods can be used for the purpose of preparing and providing compositions, combinations and containers of modified MMPs, for example tsMMPs, and use thereof for treating ECM-mediated diseases and conditions.

For example, the tsMMPs are provided in compositions, combinations and/or containers with an activator that provides the activating condition. In some examples, modified MMPs, for example tsMMPs, also are provided in compositions, combinations and/or containers with a processing agent. The activator and/or processing agent can be in the same composition or in separate compositions and in the same container or separate containers with the tsMMP. In addition, the modified MMPs, for example tsMMP, also can be combined or provided in combination, such as in containers, with other agents such as any one or more of an anesthetic, alpha-adrenergic agent, dispersing agent, or therapeutic agent. The modified MMPs, for example tsMMPs, can be provided in the same or separate composition as other agents and/or can be provided in the same or separate containers.

The modified MMPs, for example tsMMPs, can be provided as a liquid or in lyophilized form at a therapeutically effective concentration. Alternatively, the tsMMPs can be provided as a concentrated liquid, such that addition of a sufficient amount of activator results in a therapeutically effective concentration of enzyme. The enzymes can be provided as a solution or suspension or encapsulated into a suitable delivery vehicle, such as a liposome, glass particle, capillary tube, drug delivery vehicle, gelatin, gel, tablet, capsule, pill, time release coating, as well as transdermal patch preparation and dry powder inhalers or other such vehicle. The activator typically is provided as a liquid solution or suspension for administration into the interstitium either alone or following reconstitution of and/or exposure to the tsMMP. In some examples, the activator is provided exogenously and applied at the site of administration. For example, an activator can be a hot or cold pack that can be applied to the site of administration, e.g. the skin, prior to, simultaneously, subsequently or intermittently following administration of a tsMMP. As described below, kits containing these combinations and also articles of manufacture, such as containers, also are provided.

Thus, when desired, the tsMMP enzyme is subjected to activating conditions in which the enzyme is exposed to an activator to generate an enzyme that is active. Exposure to an activator can be achieved in vitro or in vivo. For example, where an activatable enzyme and activator are separately provided, they can be administered together or separately. Where administered separately, the tsMMP can be administered simultaneously, subsequently or intermittently from the activator. In another example, the tsMMP, in a lyophilized or concentrated liquid form, can be reconstituted with the activator just prior to use. In such an example, the mixture of the tsMMP and activator are administered together. Such methods of activation can be empirically determined by one of skill in the art, and may differ depending on the choice of enzyme and activator, and the method of treatment and treatment regime desired.

The tsMMP, can be provided in an article or manufacture alone or in combination with the activator. For example, if the enzyme is provided in combination with the activator, an article of manufacture can contain an enzyme, either lyophilized or in liquid form, in one compartment, and buffer that is cold or can be rendered cold in an adjacent compartment. The compartments can be separated by a dividing member. Articles of manufacture can additionally contain a processing agent. Such articles of manufacture are described elsewhere herein.

The combinations of also can further contain other agents, discussed in detail below. For example, modified MMP polypeptides, for example tsMMP, are provided in combinations containing one or more of a anesthetic, vasoconstrictor, dispersing agent or other therapeutic agent.

The following sections provide a general overview of the extracellular matrix and diseases thereof, and provide exemplary MMPs for preparation as modified MMPs, for example as temperature-sensitive activatable enzymes; methods of making such modified MMPs; exemplary modified MMPs, for example tsMMPs, that are modified MMP-1 polypeptides; compositions and combinations thereof, and methods of using modified MMP, for example modified MMP-1 polypeptides or compositions to treat ECM-mediated diseases and conditions.

### C. MATRIX METALLOPROTEASES AND THE EXTRACELLULAR MATRIX

Provided herein are modified matrix metalloproteases (MMPs). The modified MMPs include those that are activatable by temperature and degrade one or more protein components of the extracellular matrix (ECM) in a temperature controlled manner by virtue of increased activity at a permissive temperature compared to a non-permissive temperature. Hence, the modified MMPs are temperature sensitive. By virtue of such temporal in vivo activation, diseases and/or conditions of the ECM can be treated. In another example, also provided herein are modified MMPs that exhibit increased activity compared to an MMP not containing the modifications. Mutations that confer increased activity can be combined with at least one mutation that confers temperature sensitivity to generate modified MMP polypeptides that have increased activity at the permissive temperature compared to the tsMMP not containing the activity mutation. The modified MMP polypeptides, for example tsMMPs, can degrade any component of the ECM; enzyme selection can depend upon the targeted component and/or the particular disease or condition to be treated.

1. The Extracellular Matrix

The ECM makes up the connective tissue or interstitium that surrounds the spaces outside cells and the vascular and lymphatic system, thereby providing mechanical and structural support with and between different tissues. The complex and dynamic microenvironment of the ECM represents a structural and signaling system within connective tissues, such as the skin. Due to the complex nature of the ECM, it can serve diverse functions such as providing support and anchorage for cells, segregating tissues, regulating intercellular communication, and sequestering cellular growth factors. Defects or changes in the organization, or make-up, of the ECM can contribute to a number of diseases or conditions. For example, changes in the synthesis, degradation and organization of collagen fibers contribute to lipodystrophy (e.g., cellulite) and lymphedema.

The ECM is composed of fibrous structural proteins, such as collagens, polysaccharides, such as proteoglycans and hyaluronic acid, and adhesion proteins that link components of the matrix to each other and to cells. Some connective tissues, such as tendon and cartilage, are principally made up of ECM. The ECM making up the connective tissue of the skin, however, also is distributed with fibroblasts, blood vessels and other components. The ECM also serves as the space where water and its dissolved constituents move from the blood plasma to the lymphatics. The interstitial fluid is nearly isosmotic with the cytoplasm and is bicarbonate buffered providing an extracellular environment that is at neutral pH.

a. Components of the ECM

The ECM (also called the interstitial matrix) is a complex three-dimensional dynamic structure that contains numerous structural macromolecules including fibrous proteins such as collagens, elastin and fibronectin, in which glycosyaminoglycans (GAGs) form a hydrated gel-like substance. The components of the ECM are produced by resident cells, typically fibroblasts or cells of the fibroblast family, and are secreted via exocytosis where they interact with other components of the ECM. It is the variation in the relative amount and the way in which the components organize and form together that give rise to diverse connective tissues such as bone, skin or cornea (Albert et al., “Cell Junctions, Cell Adhesions and the Extracellular Matrix.” Molecular Biology of the Cell. New York: Garland Publishers, 1994. Page 972.)

i. Collagens

Collagen is the major structural constituent of connective tissues, such as the skin, and plays a role in the development and maintenance of tissue architecture, tissue strength and cell-cell interactions. Collagens include a family of structurally-related proteins of the ECM that contain one or more domains having the conformation of a collagen triple helix (Van der Rest et al. (991) FASEB J., 5:2814-2823). Collagens contain a Gly-X-Y repeating structure, which allows collagen chains to twist into a helical structure. Each collagen molecule contains three chains twisted around each other to form a triple helix, designated α1-α3. The triple helix structure provides a high mechanical strength to a collagen molecule. There are at least 27 different types of collagens, which differ in amino acid sequence and chain composition. For example, depending on the type of collagen, the three chains forming the triple helix can be the same or different. Collagens can be homotrimeric (i.e. all three polypeptide chains of the triple helix are made up of the same collagen) or can be heterotypic (i.e. fibrils made of more than one collagen type). Collagens can be divided into several families depending on the structure they form. These include fibrillar collagens (also called interstitial collagens; e.g., Type I, II, III, V and XI) and non-fibrillar collagens such as facit (e.g., Type IX, XII, XIV), short chain (e.g., Type VIII, X), basement membrane (e.g., Type IV), and other collagens (e.g., Type VI, VII, and XIII). Table 3 below sets forth common collagen types and their representative location (Van der Rest et al. (1991) FASEB J., 5:2814-2823); www.collagenlife.com/page_{—}11167323108078.html; www.indstate.edu/theme/mwking/extracellularmatrix.html).

Among the interstitial collagens, collagen molecules associate to form large fibrils, which have a distinctive banding pattern. The banding pattern results from overlap between adjacent molecules. The strength of collagen fibers is based on a multiplicity of intra- and intermolecular linkages of the collagen fibers that form the dense collagen fiber network of connective tissues. The most common of fibrillar collagens include type I, II and III collagens. Type I collagen is found in most connective tissues such as skin, bone, tendon and cornea, and is a made up of two α1(I) chains and one α2(I) chain ([α1(I)]₂ α2(I)). Type II collagen is homotrimeric ([α1(II)]₃) and is predominantly found in the cartilage. Type III collagen also is homotrimeric ([α1(III)]₃) and is predominantly found in the skin and vessels.

Not all collagens form fibril networks. For example, the basement membrane type IV collagen is non-fibrous and has non-helical interruptions in the helix, which acts as a hinge giving the molecule greater flexibility. Thus, type IV collagen forms a sheet made by a meshwork of filaments rather than by linear fibrils.

The most abundant protein of the skin is collagen, which is primarily made up of type I (80-85%) and type III (8-11%) collagen. Type I collagen associates with type III collagen to form the major collagen fibers of the dermis. The tensile strength of skin is due predominantly to these fibrillar collagen molecules, which assemble into microfibrils in a head-to-tail and staggered side-to-side lateral arrangement. Collagen molecules become cross-linked to adjacent collagen molecules, creating additional strength and stability in collagen fibers. For example, type V collagen also associates with type I/III collagen fibers, and regulates the fibril diameter. Other collagen types in the skin include, for example, type IV, type VI, type VII, type XII, type XIV and type XVII.

TABLE 3
Types of Collagens

| | | |
|---|---|---|
| Type | Molecule Composition | Representative tissue |

Fibrillar Collagens

| | | |
|---|---|---|
| I | [α1(I)]₂ [α2(I)] | Skin, bone, tendon, dentin, |
| | | ligaments, interstitial tissues |
| II | [α1(II)]₃ | Cartilage, vitreous humor |
| III | [α1(III)]₃ | Skin, muscle, blood vessels; |
| | | frequently associated with type |
| | | I |
| V | [α1(V)][α2(v)][α3(V)] | Similar to Type I, also cell |
| | | cultures, fetal tissues; |
| | | associates with Type I |
| XI | [α1(XI)][α2(XI)][α3(XI)] | Cartilage, intervertebral |
| | | cartilage and bone enamel |

Non-fibrillar collagens

| | | |
|---|---|---|
| IV | [α1(IV)]₂ [α2(IV)] | Basement membrane |
| VI | [α1(VI)[]α2(VI)][α3(VI)] | Most interstitial tissues; |
| | | associates with type I |
| VII | [α1(VII)]₃ | epithelia |
| VIII | [α1(VIII)]₃ | Unknown, some endothelial |
| | | cells |
| IX | [α1(IX)][α2(IX)][α3(IX)] | Cartilage; associates with Type |
| | | II |
| X | [α1(X)]₃ | Heterotrophic and mineralizing |
| | | cartilage |
| XII | [α1(XII)]₃ | Ligaments, tendons and tooth |
| | | enamel; interacts with types I |
| | | and III |

ii. Elastin

A network of elastic fibers in the ECM provides flexibility to tissues that require resilience to recoil after stretching, such as the skin, arteries and lungs. The main component of elastic fibers is the elastin molecule, which creates cross-links to adjacent elastin molecules. These molecules form a core of elastic fibers and are covered by fibrillin, a large glycoprotein that binds to elastin and is important for the integrity of elastic fibers.

iii. Fibronectin

Fibronectin is a glycoprotein that exists as a pair of two large subunits joined by a pair of disulfide bonds near the carboxyl termini. Each subunit contains functionally distinct domains specific for other matrix macromolecules and receptors on the surface of cells. For example, distinct domains on fibronectin bind collagen (separate domains for types I, II and III), heparin, fibrin and cell surface receptors such as integrins. Fibronectin is present in both plasma and tissue. In tissue, fibronectin functions to link together different types of ECM molecules and cells. It also contains an important cell-binding domain made up of the three amino acids, Arg-Gly-Asp (RGD), which is recognized by integrin receptors in the plasma membranes of cells. The binding of fibronectin molecules to integrin receptors on cells leads to the stimulation of signaling pathways that promote cell attachment, migration and differentiation. These characteristics allow fibronectin to play an important role in cell adhesion and to communicate signals between cells and components of the ECM.

iv. Glycosaminoglycans (GAGs)

GAGs are unbranched polysaccharide chains made of repeating disaccharide units that are strongly hydrophilic. GAGs are highly negatively charged and therefore attract osmotically active Na⁺, causing large amounts of water to be drawn into their structure to keep the ECM hydrated. GAGs, such as dermatan sulfate, typically contain multiple glycosaminoglycan chains of 70-200 sugars long (formed from repeating disaccharide units) that branch from a linear protein core. This results in GAGs occupying a huge volume relative to their mass and forming gels at very low concentrations. The hydrophilic nature of GAGs causes a swelling pressure, or turgor, which allows the ECM to withstand compression forces.

In the ECM, GAGs are attached to ECM proteins to form proteoglycans or, in the case of hyaluronic acid (also called hyaluronan), exist as a non-proteoglycan matrix component. Extracellular proteoglycans are large, highly hydrated molecules that help cushion cells in the ECM. Glycosaminoglycans such as hyaluronan contribute to the “ground substance” by creating a barrier to bulk fluid flow through the interstitial collagenous matrix by way of their viscosity and water of hydration. Proteoglycans and non-proteoglycan GAGs associate to form large polymeric complexes in the ECM. They associate with each other, and also with fibrous proteins such as collagen.

1) Proteoglyeans

There are three main types of GAGs that form proteoglycans of the ECM, including dermatan sulfate and chondroitin sulfate, heparin and heparan sulfate, and keratan sulfate. Generally, a proteoglycan is 95% carbohydrate by weight, typically in the form of long unbranched GAG chains. Besides providing hydrated space around cells, proteoglycans also regulate traffic of molecules and cells, bind signaling molecules thereby playing a role in cell activation, and bind other secreted proteins such as proteases and protease inhibitors to regulate the activities of secreted proteins (Albert et al., “Cell Junctions, Cell Adhesions and the Extracellular Matrix” Molecular Biology of the Cell. New York: Garland Publishers, 1994. pp. 972-978). For example, the heparin sulfate chains of proteoglycans bind to several different growth factors, including fibroblast growth factors (FGFs), helping them to bind to their specific cell surface receptors.

Aggrecan is a proteoglycan, which principally contains chondroitin sulfate and heparan sulfate GAGs, and is typically found in cartilage forming large aggregates with hyaluronan to provide mechanical support. Decorin is another exemplary GAG of connective tissues made up primarily of chondroitin sulfate and dermatan sulfate GAGs. It binds to type I collagen fibrils. Perlecan and betaglycan are other exemplary proteoglycans of the ECM. Not all proteoglycans are associated with the ECM: for example, serglycin is associated with secretory vesicles where it helps to package and store secretory molecules, and syndecans are found on the cell surface and act as co-receptors (Albert et al., “Cell Junctions, Cell Adhesions and the Extracellular Matrix” Molecular Biology of the Cell, New York: Garland Publishers, 1994. pp. 972-978).

Heparan sulfate proteoglycans (HSPGs) are ubiquitous macromolecules associated with the cell surface and extracellular matrix (ECM) of a wide range of cells of vertebrate and invertebrate tissues (Wight, T. N., Kinsella, M. G., and Qwarnstromn, E. E. (1992) Curr. Opin. Cell Biol., 4, 793-801; Jackson, R. L., Busch, S. J., and Cardin, A. L. (1991) Physiol. Rev., 71, 481-539; Wight, T. N. (1989) Arteriosclerosis, 9, 1-20; Kjellen, L., and Lindahl, U. (1991) Annu. Rev. Biochem., 60, 443-475; and Ruoslahti, E., and Yamaguchi, Y. (1991) Cell, 64, 867-869). The basic HSPG structure has a protein core to which several linear heparan sulfate chains are covalently attached. The polysaccharide chains are typically composed of repeating hexuronic and D-glucosamine disaccharide units that are substituted to a varying extent with N- and O-linked sulfate moieties and N-linked acetyl groups. Studies on the involvement of ECM molecules in cell attachment, growth and differentiation revealed a central role of HSPGs in embryonic morphogenesis, angiogenesis, metastasis, neurite outgrowth and tissue repair. The heparan sulfate (HS) chains, which are unique in their ability to bind a multitude of proteins, ensure that a wide variety of effector molecules cling to the cell surface. HSPGs are also prominent components of blood vessels. In large vessels they are concentrated mostly in the intima and inner media, whereas in capillaries they are found mainly in the subendothelial basement membrane where they support proliferating and migrating endothelial cells and stabilize the structure of the capillary wall. The ability of HSPGs to interact with ECM macromolecules such as collagen, laminin and fibronectin, and with different attachment sites on plasma membranes suggests a key role for this proteoglycan in the self-assembly and insolubility of ECM components, as well as in cell adhesion and locomotion.

2) Hyaluronic Acid

Hyaluronic acid (HA; also called hyaluronan) is a large GAG that attracts water, and when bound to water exists in a viscous, gel-like form. Thus, HA serves as a lubricant, holding together gel-like connective tissues. HA is a polymer of disaccharides (sometimes as many as 25,000 repeats in length) and is composed of repeating units of two modified simple sugars: glucuronic acid and N-acetyl glucosamine. HA is part of the ECM of many connective tissues. HA is found in the greatest amount in the skin with almost 50% of the body's HA found in the skin. The HA provides continuous moisture to the skin by binding up water. Decreased production of HA, such as by age, results in wrinkled and unhealthy skin.

HA, principally through its receptor CD44, also functions to regulate cell behavior during embryonic development and morphogenesis, wound healing, repair and regeneration, inflammation and tumor progression and invasion (Harada et al. (2006) J. Biol. Chem., 8:5597-5607). HA is degraded by hyaluronidases. The degradation products of HA can be found in increased amounts in damaged or growing tissues, and in a variety of inflammatory conditions. HA fragments promote angiogenesis and can stimulate cytokine production by macrophages and dendritic cells in tissue injury and skin transplant.

b. Histology of the Skin

The skin helps to maintain the body's temperature at a physiologic temperature of 37° C. The skin is composed of several distinct layers, principally the epidermis and dermis. The epidermis is a specialized epithelium derived from the ecotoderm, and beneath this is the dermis, which is a derivative of the mesoderm and is a vascular dense connective tissue. These two layers are firmly adherent to one another and form a region which varies in overall thickness form approximately 0.5 to 4 mm in different areas of the body. Beneath the dermis is a layer of loose connective tissue, which varies from areolar to adipose in character. This is referred to as the hypodermis, but is typically considered not to be part of the skin. The dermis is connected to the hypodermis by connect tissue fibers that pass from one layer to the other.

i. The Epidermis

The epidermis is the skin layer directly above the dermis, and is the surface layer of the skin. The principle function of the epidermis is to act as a protective barrier against water loss, chemical injury and invading pathogens. The epidermis is a thin layer of approximately fifteen cell layers that is about 0.1 to 1.5 millimeters thick composed primarily of keratinocytes (Inlander, Skin, New York, N.Y.: People's Medical Society, 1-7 (1998)). The epidermis is itself divided into several layers (e.g., stratum basale, stratum spinosum, stratum granulosum, stratum lucidum, stratum corneum) based on the state of differentiation of the keratinocytes. Keratinocytes originate in the basal layer from keratinocyte stem cells. As the keratinocytes grow and divide, they undergo gradual differentiation eventually reaching the stratum corneum where they form a layer of enucleated, flattened, highly keratinized cells called squamous cells (also called corneocytes). Besides being made up of corneocytes, the stratum corneum also contains sebum. The sebum is secreted by sebaceous glands, which are usually found in hair-covered areas connected to hair follicles. Sebum is a slightly acid layer that helps to hold the corneocytes together and holds moisture in. This acidity is due to the presence of amphoteric amino acids, lactic acid and fatty acids that make up sebum. Thus, the pH of the skin surface is normally between 5 and 6, typically about 5.5. Sebum acts to waterproof hair and skin, and keep them from becoming dry, brittle and cracked, and it also inhibits the growth of microorganisms on skin. The term “acid mantle” refers to the presence of the water-soluble substances on most regions of the skin.

ii. The Dermis

The connective tissue of the skin is called the dermis. The dermis is 1.5 to 4 millimeters thick. In the skin, the dermis contains ECM components; the main protein components are collagen and elastin. The dermis also is home to most of the skin's structures, including sweat and oil glands that secrete substances through openings in the skill called pores, or comedos, hair follicles, nerve endings, and blood and lymph vessels (Inlander, Skin, New York, N.Y.: People's Medical Society, 1-7 (1998)). In addition, the dermis contains blood vessels that play a role in temperature regulation.

iii. The Hypodermis

Below the dermis is the hypodermis, which is a fatty layer and is the deepest layer of the skin. It acts as an insulator for body heat conservation and as a shock absorber for organ protection (Inlander, Skin, New York, N.Y.: People's Medical Society, 1-7 (1998)). In addition, the hypodermis also stores fat for energy reserves.

c. Diseases of the ECM

Certain diseases and conditions result from defects or changes in the architecture of the extracellular matrix due to aberrant expression or production of ECM components. For example, in some inflammatory conditions such as occur upon wound healing, cytokines are secreted, which stimulate fibroblasts to secrete ECM components such as collagen. The ECM components accumulate and become locally deposited, resulting in a wide range of fibrotic conditions. Matrix deposition is a frequent feature in many chronic inflammatory diseases and in other diseases and conditions. Included among these are collagen-mediated disease conditions such as, but not limited to, scars such as keloid and hypertrophic scars, Duputyren's syndrome, Peyronie's disease and lymphedema. Cellulite also is a prominent disease of the ECM that, in addition to increased adipogenicity, is characterized by alterations in the connective tissue matrix resulting in an abnormal fibrous septae network of collagen (Rawlings et al. (2006) Int. J. Cos. Science, 28:175-190).

Diseases and conditions of the ECM that are characterized by aberrant expression or overproduction of matrix components, resulting in their accumulation and unwanted deposition, can be treated by the tsMMPs provided herein. By virtue of the temporal activation of such enzymes upon in vivo administration, the treatment of such diseases and conditions is regulated to limit the enzymatic degradation of the matrix components. For example, by limiting the duration of action of matrix degradation, unwanted side effects associated with uncontrolled protein degradation is minimized.

2. Matrix Metalloproteases

Provided herein are modified MMPs that are temperature sensitive (tsMMPs). The modified MMPs include those that exhibit increased activity at a lower temperature then a higher temperature and also those that exhibit increased activity at a higher temperature then a lower temperature. The tsMMPs are provided as compositions, combinations and containers, and can be used in methods, processes and uses to treat ECM-mediated diseases or conditions. MMPs are matrix-degrading enzymes that degrade protein components of the extracellular matrix (ECM), including, but not limited to, collagen, elastin, fibronectin and proteoglycans. By virtue of their ability to cleave one or more ECM components, activatable tsMMPs provided herein can be used to modify the matrix of tissues, particularly those exhibiting structural defects or changes due to excess of one or more ECM protein or unwanted accumulation of fibrous tissue rich in one or more ECM protein, such as collagen. Thus, such enzymes are useful in treating diseases or conditions in which ECM proteins are involved.

a. Function

Matrix metalloproteinases (MMPs) are a family of zinc-dependent and calcium-dependent endopeptidases. For example, MMPs contain an active site Zn²⁺ required for activity. Most MMPs are involved in degradation of the extracellular matrix. For example, many of these enzymes can cleave components of the basement membrane and extracellular matrix. They are involved in tissue remodeling, for example, in processes such as wound healing, pregnancy and angiogenesis. In addition, MMPs also can process a number of cell-surface cytokines, receptors and other soluble proteins. The proteolytic activity of MMPs act as an effector mechanism of tissue remodeling in physiologic and pathologic conditions, and as modulator of inflammation. The excess synthesis and production of MMPs leads to accelerated degradation of the ECM which is associated with a variety of diseases and conditions such as, for example, bone homeostasis, arthritis, cancer, multiple sclerosis and rheumatoid arthritis. In the context of neuroinflammatory diseases, MMPs have been implicated in processes such as (a) blood-brain barrier (BBB) and blood-nerve barrier opening, (b) invasion of neural tissue by blood-derived immune cells, (c) shedding of cytokines and cytokine receptors, and (d) direct cellular damage in diseases of the peripheral and central nervous system (Leppert et al. Brain Res. Rev. 36(2-3): 249-57 (2001); Borkakoti et al. Prog. Biophys. Mol. Biol. 70(1): 73-94 (1998)). The enzymes are specifically regulated by endogenous inhibitors called tissue inhibitors of matrix metalloproteases (TIMPs).

b. Structure and Activation

Generally, MMPs contain three common domains: the pro-peptide, the catalytic domain and the hemopexin-like C-terminal domain. MMPs are synthesized as zymogens. Zymogen activation prevents unwanted protein degradation that could occur if proteases were always present in active form. Generally, zymogens contain N-terminal portions (or prosegments or proregions or propeptide) that sterically block the active site of the protease and prevent access of substrates to the active site of the protease. The propeptide also acts to stabilize the polypeptide. The propeptide of zymogen forms of MMPs range in size from about 80-100 residues in length. The propeptide of MMPs contains a cysteine residue generally contained in the conserved sequence PRCxxPD (with the exception of MMP-23, which contains the critical cysteine and different surrounding amino acids). The cysteine residue interacts with the zinc in the active site and prevents binding and cleavage of the substrate, thereby keeping the enzyme in an inactive form. Thus, upon secretion from a preproenzyme form, the proenzyme (containing the propeptide) is inactive. For example, in MMP-1 the propeptide cysteine residue corresponds to amino acid residue 73 in the sequence of amino acids set forth in SEQ ID NO:2.

MMPs require processing for activation. Generally, processing involves removal of the propeptide and/or conformational changes of the enzyme to generate a processed mature form. Processing of the enzyme by removal of the propeptide is required for activity of MMPs. For normal MMPs (e.g. wildtype) that are not conditionally active as provided herein, the processed mature form is an active enzyme. Thus, it is understood that wildtype MMPs in their processed mature form are enzymatically active, and thus for these enzymes this is the active form. tsMMPs provided herein, however, also additionally require the permissive temperature condition to be fully active.

Processing (and thereby activation) can be induced by processing agents such as proteases, including other previously activated MMPs; by chemical activation, such as thiol-modifying agents (4-aminophenylmercuric acetate, HgCl₂ and N-ethylmaleimide), oxidized glutathione, SDS, chaotropic agents and reactive oxygens; and by low pH or heat treatment. For example, Table 4 below lists exemplary processing agents (see also Visse et al. (2003) Circ. Res., 92:827-839; Khan et al. (1998) Protein Science, 7:815-836; Okada et al. (1988) Biochem J., 254:731-741; Okada & Nakanashi (1989) FEBS Lett., 249:353-356; Nagase et al. (1990) Biochemistry, 29:5783-5789; Koklitis et al. (1991) Biochem J., 276:217-221; Springman et al. (1990) PNAS, 87:364-8; Murphy et al. (1997) Matrix Biol., 15:511-8).

TABLE 4
Zymogen Activators (i.e. processing agents)

Proteolytic Compounds

| | |
|---|---|
| Proteases | Plasmin |
| | Plasma kallikrein |
| | Trypsin-1 (Trypsin I) |
| | Trypsin-2 (Trypsin II) |
| | Neutrophil elastase |
| | Cathepsin G |
| | Tryptase |
| | Chymase |
| | Proteinase-3 |
| | Furin |
| | uPA |
| | MMPs, including MMP-1, MMP-2, |
| | MMP-3,MMP-7, MMP-10, MMP-26, |
| | and MTI-MMP |

Non-Proteolytic Compounds

| | |
|---|---|
| Thiol-modifying Agents | 4-aminophenylmercuric acetate (AMPA) |
| | HgCl₂ |
| | N-ethylmaleimide |
| Conformational Perturbants | Sodium dodecyl sulfate (SDS) |
| | Chaotropic agents |
| Other Chemical Agents | Oxidized glutathione (GSSG) |
| | Reactive oxygen |
| | Au(I) salts |

Other Activating Conditions

Acidic pH
Heat

MMP activation occurs in a stepwise manner. For example, activation by proteases involves a first proteolytic attack of a bait region (corresponding to amino acids 32-38 of proMMP-1 (SEQ ID NO:2)), an exposed loop region found between the first and second helices of the pro-peptide. The sequence of the bait region confers cleavage specificity. Following initial cleavage, the remaining propeptide is destabilized allowing for intermolecular processing by other partially active MMP intermediates or active MMPs. For example, the protease plasmin activates both proMMP-1 and proMMP-3. Once activated, MMP-3 effects the final activation of proMMP-1. Alternatively, activation by chemicals, for example APMA, initially causes the modification of the propeptide cysteine residue, which in turn causes partial activation and intramolecular cleavage of the propeptide. The remaining segment of propeptide is then processed by other proteases or MMPs.

Metalloproteinases contain a Zn²⁺ ion at the active center of the enzyme required for catalytic activity. Generally, these enzymes have a common zinc binding motif (HExxHxxGxxH) in their active site, and a conserved methionine turn following the active site. The zinc binding motif at the active site of a metalloproteinase includes two histidine residues whose imidazole side-chains are ligands to the Zn²⁺. During catalysis, the Zn²⁺ promotes nucleophilic attack on the carbonyl carbon by the oxygen atom of a water molecule at the active site. An active site base (a glutamate residue in carboxypeptidases) facilitates this reaction by extracting a proton from the attacking water molecule. Thus, the glutamate (E) residue activates a zinc-bound H₂O molecule, thereby providing the nucleophile that cleaves peptide bonds. Mutation of any one of the histidines ablates catalytic activity. The catalytic domain also contains two calcium binding sites on either side of the zinc binding motif. The Ca²⁺ binding sites are characterized as being a highly conserved Glu- and Asp-rich region.

Many MMPs also contain a flexible proline-rich hinge region, which is up to about 75 amino acids long, but has no known structure. MMPs also contain a hemopexin-like C-terminal domain that functions in substrate recognition and also interacts with inhibitors, in particular tissue inhibitor of metalloproteinases (TIMPs). MMP-7, MMP-23 and MMP-26 do not contain a hemopexin domain. MMP-2 and MMP-9 also contain an insert in the catalytic domain made up of three tandem repeats of fibronectin type II modules that confer gelatin-binding properties to these enzymes.

There are over 25 MMPs known and they are grouped into different families depending on function, substrate specificity and/or sequence similarity. The families of MMPs include collagenases, gelatinases, stromelysins and matrilysins. Among the various families, some MMPs contain additional domains. For example, membrane-type MMPs contain a transmembrane or a GPI-anchoring domain. Exemplary MMPs are set forth in Table 5. The sequence identifiers (SEQ ID NO) for the nucleotide sequence and encoded amino acid sequence of the precursor polypeptide for each of the exemplary proteases is depicted in the Table. The sequence identifiers (SEQ ID NO) for the amino acid sequence of the preproprotein and the zymogen-activated processed mature form of the protein (lacking the propeptide) also are depicted in the Table. The location of domains also is indicated. Those of skill in the art are familiar with such domains and can identify them by virtue of structural and/or functional homology with other such domains. It is understood that polypeptides and the description of domains thereof are theoretically derived based on homology analysis and alignments with similar polypeptides. Thus, the exact locus can vary, and is not necessarily the same for each polypeptide. Variations of MMPs also exist among allelic and species variants and other variants known in the art, and such variants also are contemplated for modification as activatable tsMMPs as described herein below. The Table also sets forth exemplary ECM target substrates for each enzyme. Reference to such substrates is for reference and exemplification, and are not intended to represent an exhaustive list of all target substrates. One of skill in the art knows or can empirically determine ECM target substrates for a desired enzyme using routine assays, such as any described herein.

TABLE 5
Metalloprotease

| | | Enzyme | | | | |
|---|---|---|---|---|---|---|
| | | databank | | | | |
| | | access | | SEQ ID NO | | |
| | | code (EC) | | | | Mature |
| | | www.expasy.ch/ | | | | (processed |
| | | sprot/ | Genbank | Precursor | | form) |
| Protease | Substrate | elizyme.html | No. | nt | Aa | aa |

Collagenases:

| | | | | | | |
|---|---|---|---|---|---|---|
| MMP-1 | collagen I, II, | 3.4.24.7 | P03956, | 708 | 1 | 709 |
| (collagenase-1) | III, VII, VIII, | | NM_002421 | | (ss aa 1-19; | |
| | X, XI, gelatin, | | | | pp aa 20-99) | |
| | proteoglycan, | | | | | |
| | fibronectin, | | | | | |
| | glycoprotein | | | | | |
| MMP-8 | collagen I, II, | 3.4.24.34 | P22894 | 710 | 711 | 712 |
| (collagenase-2) | III, aggrecan | | NM_002424 | | (ss aa 1-20; | |
| | | | | | pp aa 21-100) | |
| MMP-13 | collagen I, II, | 3.4.24.- | P45452 | 713 | 714 | 715 |
| (collagenase-3) | III, IV, VI, | | NM_002427 | | (ss aa 1-19; | |
| | IX, X, XIV, | | | | pp aa 20-103) | |
| | gelatin, | | | | | |
| | proteoglycan, | | | | | |
| | fibronectin, | | | | | |
| | glycoprotein | | | | | |
| MMP-18 | collagen I | 3.4.24.- | Xenopus | 716 | 717 | 718 |
| (collagenase-4) | | | laevis | | (ss aa 1-17; | |
| | | | O13065 | | pp aa 18-99) | |

Gelatinases:

| | | | | | | |
|---|---|---|---|---|---|---|
| MMP-2 | gelatins, | 3.4.24.24 | P08253 | 719 | 720 | 721 |
| (gelatinase | collagen I, II, | | NM_004530 | | (ss aa 1-29; | |
| A) | III, IV, V, | | | | pp 30-109) | |
| | VII, X, XI, | | | | | |
| | elastin, | | | | | |
| | fibronectin, | | | | | |
| | laminin, | | | | | |
| | proteoglycan, | | | | | |
| | glycoprotein | | | | | |
| MMP-9 | gelatin, | 3.4.24.35 | P14780 | 722 | 723 | 724 |
| (gelatinase | collagen IV, | | NM_004994 | | (ss aa 1-19; | |
| B) | V, VI, XIV, | | | | pp aa 20-93) | |
| | elastin, | | | | | |
| | laminin, | | | | | |
| | proteoglycan, | | | | | |
| | glycoprotein | | | | | |

Stromelysins:

| | | | | | | |
|---|---|---|---|---|---|---|
| MMP-3 | fibronectin, | 3.4.24.17 | P08254 | 725 | 726 | 727 |
| (stromelysin-1) | elastin, | | NM_00242 | | (ss aa 1-17; | |
| | laminin, | | | | pp aa 18-99) | |
| | gelatin, | | | | | |
| | proteoglycan, | | | | | |
| | glycoprotein, | | | | | |
| | collagen III, | | | | | |
| | IV, V, VII, | | | | | |
| | IX, X, XI | | | | | |
| MMP-10 | collagen III, | 3.4.24.22 | P09238 | 728 | 729 | 730 |
| (stromelysin-2) | IV, V, elastin, | | NM_002425 | | (ss aa 1-17; | |
| | gelatin, | | | | pp aa 18-98) | |
| | fibronectin, | | | | | |
| | aggrecan | | | | | |
| MMP-11 | Gelatin, | 3.4.24.- | P24347 | 731 | 732 | 733 |
| (stromelysin-3) | fibronectin, | | X57766 | | (ss aa 1-31; | |
| | laminin, | | | | pp aa 32-97) | |
| | collagen IV | | | | | |

Matrilysins:

| | | | | | | |
|---|---|---|---|---|---|---|
| MMP-7 | fibronectin, | 3.4.24.23 | P09237 | 734 | 735 | 736 |
| (matrilysin) | laminin, | | NM_002423 | | (ss aa 1-17; | |
| | elastin, | | | | pp aa 18-94) | |
| | gelatin, | | | | | |
| | collagen I, IV, | | | | | |
| | proteoglycan, | | | | | |
| | glycoprotein | | | | | |
| MMP-26 | collagen IV, | 3.4.24.- | Q9NRE1 | 737 | 738 | 739 |
| (matrilysin-2) | fibronectin, | | NM_021801 | | (ss aa 1-17; | |
| | gelatin, | | | | pp aa 18-89) | |
| | proteoglycan | | | | | |

Metalloelastase:

| | | | | | | |
|---|---|---|---|---|---|---|
| MMP-12 | elastin, | 3.4.24.65 | P39900 | 740 | 741 | 742 |
| (metalloelastase) | fibronectin, | | NM_002426 | | (ss aa 1-16; | |
| | laminin, | | | | pp aa 17-105) | |
| | collagen I, IV, | | | | | |
| | V, gelatin, | | | | | |
| | proteoglycan, | | | | | |
| | glycoprotein | | | | | |

Membrane-type MMPs:

| | | | | | | |
|---|---|---|---|---|---|---|
| MMP-14 | Collagen I, II, | 3.4.24.80 | P50281 | 743 | 744 | 745 |
| (MT1-MMP) | III, gelatin, | | NM_004995 | | (ss aa 1-20; | |
| Transmembrane | aggrecan, | | | | pp aa 21-111) | |
| | fibronectin, | | | | | |
| | laminin, | | | | | |
| | proteoglycan, | | | | | |
| | glycoprotein | | | | | |
| MMP-15 | aggrecan, | EC | P51511 | 746 | 747 | 748 |
| (MT2-MMP) | fibronectin, | 3.4.24.- | NM_002428 | | (ss aa 1-41; | |
| Transmembrane | laminin, | | | | pp aa 42-13 1) | |
| | glycoprotein | | | | | |
| MMP-16 | Collagen III, | EC | P51512 | 749 | 750 | 751 |
| (MT3-MMP) | fibronectin, | 3.4.24.- | NM_005941 | | (ss aa 1-31; | |
| Transmembrane | laminin, | | | | pp aa 32-119) | |
| | gelatin, | | | | | |
| | proteoglycan | | | | | |
| MMP-17 | gelatin | EC | Q9ULZ9 | 752 | 753 | 754 |
| (MT4-MMP) | | 3.4.24.- | AB021225 | | (ss aa 1-38; | |
| GPI anchor | | | | | pp aa 39-128) | |
| MMP-24 | fibronectin, | EC | Q9Y5R2 | 755 | 756 | 757 |
| (MT5-MMP) | gelatin, | 3.4.24.- | NM_006690 | | (ss aa 1-52; | |
| Transmembrane | proteoglycan | | | | pp aa 53-155) | |
| MMP-25 | collagen IV, | EC | Q9NPA2 | 758 | 759 | 760 |
| (MT6-MMP) | gelatin, | 3.4.24.- | NM_022468 | | (ss aa 1-21; | |
| GPI anchor | fibronectin, | | | | pp aa 22-107) | |
| | proteoglycan | | | | | |

Enamelysin:

| | | | | | | |
|---|---|---|---|---|---|---|
| MMP-20 | aggrecan | EC | O60882 | 761 | 762 | 763 |
| (enamelysin) | | 3.4.24.- | Y12779 | | (ss aa 1-22; | |
| | | | | | pp aa 23-107) | |

Other:

| | | | | | | |
|---|---|---|---|---|---|---|
| MMP-19 | collagen IV, | EC | Q99542 | 764 | 765 | 766 |
| | gelatin, | 3.4.24.- | NM_002429 | | (ss aa 1-18; | |
| | laminin, | | | | pp aa 19-97) | |
| | aggrecan, | | | | | |
| | fibronectin, | | | | | |
| | glycoprotein | | | | | |
| MMP-21 | gelatin | EC | Q8N119 | 767 | 768 | 769 |
| | | 3.4.24.- | NM_147191 | | (ss aa 1-24; | |
| | | | | | ppaa25-144) | |
| MMP-23 | gelatin | EC | O75900 | 770 | 771 | 772 |
| CA-MMP | | 3.4.24.- | AJ005256 | | | |
| MMP-27 | gelatin | EC | Q9H30 | 6 773 | 774 | 775 |
| CMMP | | 3.4.24.- | NM_022122 | | (ss aa 1-17; | |
| | | | | | pp aa 18-98) | |
| MMP-28 | | EC | Q9H239 | 776 | 777 | 778 |
| (epilysin) | | 3.4.24.- | NM_024302 | | (ss aa 1-22; | |
| | | | | | pp aa 23-122) | |

3. Matrix Metalloprotease-1 (MMP-1)

MMP-1 (also called collagenase) is encoded by a nucleic acid molecule set forth in SEQ ID NO:708 resulting in a pre-procollagenase (SEQ ID NO:1), which is co-translationally processed to generate a procollagenase zymogen form (SEQ ID NO:2). Procollagenase contains a propeptide of 80 amino acids (corresponding to amino acid residues 1-80 of the sequence of amino acids set forth in SEQ ID NO:2), a catalytic domain of 162 amino acids (corresponding to amino acid residues 81-242 of the sequence of amino acids set forth in SEQ ID NO:2), a 16-residue linker (corresponding to amino acid residues 243-258 of the sequence of amino acids set forth in SEQ ID NO:2) and a hemopexin (Hpx) domain of 189 amino acid residues (corresponding to amino acid residues 259-450 of the sequence of amino acids set forth in SEQ ID NO:2). Upon processing, the propeptide is removed, resulting in a processed mature form having a sequence of aminoacids set forth in SEQ ID NO: 709.

As noted above, MMP-1 cleaves collagen type I and collagen type III, which are the most abundant protein of the skin. These collagen types are associated with many of the conditions of the ECM as described herein in Section 1. In contrast, other collagens, for example collagen type IV, is a major component of the basal lamina of blood vessels. Hence, targeting of type IV collagen, for example, can lead to leaky blood vessels, which can be a side effect of treatments that are meant to target the extracellular matrix as described herein. For example, bacterial collagenase, a known treatment for cellulite, can induce haemorrhages (see e.g. Vargaftig et al. (2005) Inflammation Research, 6:627-635). Thus, an advantage of the use of MMP-1, and in particular tsMMP-1 that can be conditionally or temporally controlled, as a therapeutic agent to treat conditions of the ECM is that it does not cleave type IV collagen.

### D. MODIFIED MATRIX METALLOPROTEASE-1 POLYPEPTIDES

Provided herein are modified MMP-1 polypeptides. In one example, modified MMP-1 polypeptides provided herein exhibit temperature sensitivity, whereby the modified polypeptide exhibits higher activity at a permissive temperature than a non-permissive temperature. Also provided herein are modified MMP-1 polypeptides that exhibit increased activity compared to the unmodified MMP-1 not containing the modification (e.g. wildtype) at both permissive and non-permissive temperatures. In an additional example, provided herein are modified MMP-1 polypeptides that exhibit modifications that both increase temperature sensitivity and activity.

Modifications provided herein of a starting, unmodified reference polypeptide include amino acid replacements or substitutions, additions or deletions of amino acids, or any combination thereof. For example, modified MMP-1 polypeptides include those with 1, 2, 3, 4, 5, 6, 7, 8, 9, 10, 11, 12, 13, 14, 15, 16, 17, 18, 19, 20 or more modified positions. Also provided herein are modified MMP-1 polypeptides with two or more modifications compared to a starting reference MMP-1 polypeptide. Modified MMP-1 polypeptides include those with 2, 3, 4, 5, 6, 7, 8, 9, 10, 11, 12, 13, 14, 15, 16, 17, 18, 19, 20 or more modified positions. In some examples, modified MMP-1 polypeptide provided herein contain only a single modification. In other examples, modified MMP-1 polypeptides provided herein contain two, three, four, five or six modifications. In additional examples, any modification(s) provided herein can be combined with any other modification known to one of skill in the art so long as the resulting modified MMP-1 polypeptide retains enzymatic activity when it is in its processed mature form. Where the modified MMP-1 contains a mutation conferring temperature sensitivity, the enzymatic activity of such combination mutant is greater at the permissive temperature compared to the non-permissive temperature. Modified MMP-1 polypeptides provided herein can be assayed for enzymatic activity under various conditions (e.g. permissive and non-permissive temperatures) to identify those that retain enzymatic activity.

Modifications in an MMP-1 polypeptide can be made to any form of an MMP-1 polypeptide, including inactive (e.g. zymogen) or processed mature forms (activated form), allelic and species variants, splice variants, variants known in the art, or hybrid or chimeric MMP-1 polypeptides. For example, modifications provided herein can be made in a precursor MMP-1 polypeptide set forth in SEQ ID NO:1, an inactive pro-enzyme MMP-1 containing the propeptide set forth in SEQ ID NO:2, a mature MMP-1 polypeptide lacking the propeptide set forth in SEQ ID NO:709, or any species, allelic or modified variant and active fragments thereof that has 40%, 50%, 60%, 70%, 80%, 90%, 91%, 92%, 93%, 94%, 95%, 96%, 97%, 98%, or 99% sequence identity to any of the MMP-1 polypeptides set forth in SEQ ID NOS: 1, 2 or 709. Modifications also can be in an MMP-1 polypeptide lacking one or more domains, so long as the MMP-1 polypeptide retains enzymatic activity. For example, modifications can be in an MMP-1 polypeptide that includes only the catalytic domain (corresponding to amino acids 81-242) of the proenzyme MMP-1 polypeptide set forth in SEQ ID NO:2). Modifications also can be made in an MMP-1 polypeptide lacking all or a portion of the proline rich linker (corresponding to amino acids 243-258 of the proenzyme MMP-1 polypeptide set forth in SEQ ID NO:2) and/or lacking all or a portion of the hemopexin binding domain (corresponding to amino acids 259-450 of the proenzyme MMP-1 polypeptide set forth in SEQ ID NO:2). Allelic variants and other variants of MMP-1 polypeptides include, but are not limited to, any of MMP-1 polypeptide containing any one or more amino acid variant set forth in SEQ ID NO:3506 and 3549. Exemplary species variants for modification herein include, but are not limited to, pig, rabbit, bovine, horse, rat, and mouse, for example, set forth in any of SEQ ID NOS:3459-3464.

Modifications in an MMP-1 polypeptide provided herein, for example in an MMP-1 containing a modification to confer temperature sensitivity and/or increased activity, can be made to an MMP-1 polypeptide that also contains other modifications, such as those described in the art, including modification of the primary sequence and modifications not in the primary sequence of the polypeptide. It is understood that modifications in an allelic or species variant or other variant include modification in any form thereof such as an active or inactive form, a form including only the catalytic domain, or a form lacking all or a portion of the proline rich linker or the hemopexin binding domain. As discussed herein below, corresponding MMP-1 modifications can be made to similar forms of other MMP polypeptides.

Hence, the resulting modified MMP-1 polypeptides include those that are inactive zymogen proenzymes and those that are processed mature polypeptides. For example, any modified polypeptide provided herein that is a zymogen proenzyme can be activated by a processing agent to generate a processed mature MMP-1 polypeptide. Activity of MMP-1 polypeptides are typically exhibited in its processed mature form following cleavage of the propeptide and/or intermolecular and intramolecular processing of the enzyme to remove the propeptide (see e.g. Visse et al. (2003) Cir. Res., 92:827-839). As noted elsewhere herein, tsMMP's require permissive temperature to be fully active.

The modifications provided herein can be made by standard recombinant DNA techniques such as are routine to one of skill in the art. Any method known in the art to effect mutation of any one or more amino acids in a target protein can be employed. Methods include standard site-directed mutagenesis (using e.g. a kit, such as QuikChange available from Stratagene) of encoding nucleic acid molecules, or by solid phase polypeptide synthesis methods.

Other modifications that are or are not in the primary sequence of the polypeptide also can be included in a modified MMP-1 polypeptide, or conjugate thereof, including, but not limited to, the addition of a carbohydrate moiety, the addition of a polyethylene glycol (PEG) moiety, the addition of an Fc domain, etc. For example, such additional modifications can be made to increase the stability or half-life of the protein.

Exemplary of such modified MMP-1 polypeptides are set forth in any of SEQ ID NOS:3-705, 779-3458 and 3532 and processed mature forms and other forms thereof, and allelic and species variants thereof.

1. Temperature-Sensitive Matrix

Metalloprotease-1 (tsMMP-1) Mutants

Provided herein are tsMMP-1 polypeptides that are temperature sensitive by virtue of modifications in the primary sequence of the polypeptide compared to an unmodified MMP-1 polypeptide. The tsMMP-1 polypeptides exhibit increased enzymatic activity at a permissive temperature compared with activity of the tsMMP-1 polypeptide at a non-permissive temperature. For example, tsMMP-1 polypeptides provided herein exhibit increased enzymatic activity at a low temperature that is less then 37° C., for example, that is at or about 18° C., 19° C., 20° C., 21° C., 22° C., 23° C., 24° C., 25° C., 26° C., 27° C., 28° C., 29° C. or 30° C., in particular at or about 18° C. to 25° C., for example at or about 25° C. compared to a non-permissive high temperature that is at or about 34° C., 35° C., 36° C., 37° C., 38° C. or 39° C., in particular at or about 34° C. or 37° C. Due to the temperature-dependent activity of tsMMP-1 polypeptides, the activity of MMP-1 can be conditionally controlled, thereby temporally regulating activation to prevent prolonged and unwanted degradation of the ECM. In particular, such tsMMP-1 polypeptides can be used in uses, processes or methods to treat diseases or conditions of the ECM, for example, to treat collagen-mediated diseases or conditions such as cellulite.

The tsMMP-1 polypeptides provided herein have a ratio of activity at a permissive temperature compared to a non-permissive temperature that is or is about 1.1, 1.2, 1.3, 1.4, 1.5, 1.6, 1.7, 1.8, 1.9, 2.0, 2.5, 3.0, 3.5, 4.0, 4.5, 5, 5.5, 6, 6.5, 7, 7.5, 8, 8.5, 9, 9.5, 10, 15, 20, 30, 40, 50, 60, 70, 80, 90, 100 or more. Thus, the activity of tsMMP-1 polypeptides provided herein at the non-permissive temperature is or is about 70%, 65%, 60%, 55%, 50%, 45%, 40%, 35%, 30%, 25%, 20%, 15%, 10%, 5%, 4%, 3%, 2%, 1%, 0.5% or less of the activity at a permissive temperature. tsMMPs-1 polypeptides provided herein retain one or more activities of wildtype MMP-1 polypeptide at the permissive temperature, for example, enzymatic activity for cleavage of an ECM component such as collagen. Typically, such activity is substantially unchanged (less than 1%, 5%, 10%, 20% or 30% changed) compared to a wildtype or starting protein. In other examples, the activity of a modified MMP-1 polypeptide is increased or is decreased as compared to a wildtype or starting MMP-1 polypeptide. Activity is assessed at the permissive temperature and is compared to the activity of a starting, unmodified MMP-1 polypeptide (i.e. polypeptide not containing the modification) at the permissive temperature or a non-permissive temperature. For example, a tsMMP-1 polypeptide provided herein retains an activity at the permissive temperature that is or is about 10%, 20%, 30%, 40%, 50%, 60%, 70%, 80%, 90%, 100%, 110%, 120%, 140%, 150% or more the activity of wildtype MMP-1 at the permissive temperature or non-permissive temperature. Activity can be assessed in vitro, ex vivo or in vivo and can be compared to that of the unmodified MMP-1 polypeptide, such as for example, an inactive MMP-1 polypeptide set forth in SEQ ID NO:2 activated by a processing agent, or any other MMP-1 polypeptide known to one of skill in the art that is used as the starting material. As discussed elsewhere herein, it is understood that the zymogen inactive form of an MMP-1 or a modified MMP-1 must be processed to a processed mature form required for activity before use or measurement of an activity.

### Exemplary Temperature Sensitive Modifications

Provided herein are modified tsMMP-1 polypeptides containing one or more amino acid modifications in a starting, unmodified MMP-1 polypeptide. Typically, the modification is an amino acid replacement. The amino acid replacement or replacements can be at any one or more positions corresponding to any of the following positions: 84, 85, 95, 98, 99, 100, 103, 104, 105, 106, 109, 110, 111, 112, 118, 123, 124, 126, 147, 150, 151, 152, 153, 155, 156, 158, 159, 170, 171, 176, 178, 179, 180, 181, 182, 183, 185, 187, 188, 189, 190, 191, 192, 194, 195, 197, 198, 206, 207, 208, 210, 211, 212, 218, 223, 227, 228, 229, 230, 233, 234, 237, 240, 251, 254, 255, 256, 257, 258, 259 of an unmodified MMP-1 polypeptide having a sequence of amino acids set forth in SEQ ID NO:2, or at a corresponding position in an allelic or species variant or other variant of an MMP-1 polypeptide that has at least or at least about 60%, 70%, 75%, 80%, 85%, 90%, 91%, 92%, 93%, 94%, 95%, 96%, 97%, 98%, 99% or more sequence identity to an MMP-1 polypeptide set forth in SEQ ID NO:2. Amino acid replacements include replacement of amino acids to an acidic (D or E); basic (H, K or R); neutral (C, N, Q, T, Y, S, G) or hydrophobic (F, M, W, I V, L A, P) amino acid residue. For example, amino acid replacements at the noted positions include replacement by amino acid residues E, H, R, C, Q, T, S, G, M, W, I, V, L, A, P, N, F, D, Y or K.

Such modified MMP-1 polypeptides include MMP-1 polypeptides that are temperature sensitive by virtue of increased activity at the permissive temperature of 25° C. compared to the non-permissive temperatures of 34° C. or 37° C. For example, modified MMP-1 polypeptides provided herein can include polypeptides having an amino acid modification corresponding to any one or more modifications of T84F (i.e. replacement of T by F at a position corresponding to position 84 of an MMP-1 polypeptide set forth in SEQ ID NO:2), E85F, L95K, L95I, R98D, I99Q, E100V, E100R, E100S, E100T, E100F, E100I, E100N, T103Y, P104A, P104M, D105A, D105F, D105G, D105I, D105L, D105N, D105R, D105S, D105T, D105W, D105E, L106C, L106S, A109H, D110A, V111R, D112S, A118T, S123V, N124D, T126S, G147P, R150P, R150V, R150D, R150I, R150H, D151G, N152A, N152S, S153T, F155L, F155A, D156H, D156L, D156A, D156W, D156V, D156K, D156T, D156R, D156M, P158T, P158G, P158K, P158N, G159V, G159T, G159M, G159I, G159W, G159L, G159C, P170D, P170A, G171P, G171E, G171D, A176F, A176W, F178T, F178L, D179N, D179V, D179C, E180Y, E180R, E180T, E180F, E180G, E180S, E180N, E180D, D181T, D181L, D181K, D181C, D181G, E182T, E182Q, E182M, E182G, R183G, R183S, T185R, T185Y, T185H, T185G, T185V, T185Q, T185A, T185E, T185D, N187R, N187M, N187W, N187F, N187K, N187I, N187A, N187G, N187C, N187H, F188V, R189N, R189T, R189Q, E190G, E190Y, E190D, Y191V, N192H, N192S, N192D, N192C, H194P, R195C, R195W, R195L, R195G, R195Q, R195A, R195D, R195V, A197C, A197V, A198G, A198L, A198M, G206A, G206S, L207R, L207V, L207I, L207G, S208R, S208L, S210V, S210A, T211L, D212G, D212H, Y218S, F223C, F223E, F223G, F223A, F223S, F223K, F223M, V227C, V227D, V227E, V227L, V227S, V227W, V227G, V227H, V227Q, V227R, Q228P, L229A, L229T, L229I, A230V, D233E, I234A, I234T, I234E, I234Q, I237L, I237W, I237N, I240S, I240A, I240C, I251 S, I251W, Q254S, T255H, P256C, K257P, K257T, A258P and C259Q. Exemplary modified MMP-1 polypeptides have a sequence of amino acids set forth in any of SEQ ID NOS:6, 18, 22, 25, 27, 29, 31-33, 35-36, 38-39, 41,43,55-56, 59, 70, 95-96, 99-101, 105, 110-111, 113-115, 122, 125, 129-133, 148, 150, 159-160, 170, 174, 177, 179, 181-185, 195, 197, 200, 203, 209, 218-219, 222, 224, 231-233, 235, 238-239, 241, 246, 248, 252-255, 260-264, 267, 269, 273, 275, 279, 282, 284-286, 299, 301, 305, 317, 324, 341, 343, 354, 365, 367, 369, 374-376, 381, 383-385, 387-388, 390, 393-394, 397, 399, 420, 429, 436, 438, 440, 460, 466-467, 476, 483, 488, 495, 500, 502, 504, 506, 508, 511-512, 524, 543, 554-555, 572-573, 581, 583, 607, 611, 613, 616, 620, 648, 653, 660, 664-665, 669, 678, 703, 847, 866, 1083, 1109, 1172, 1177, 1183, 1188, 1237, 1271, 1277, 1301, 1414, 1516, 1520, 1567, 1975, 2023, 2031, 2075, 2078, 2080, 2083, 2281, 2299, 2403, 2411, 2423-2424, 2486, 2495-2497, 2552, 2563, 2703, 2715, 2753, 3066, 3074, 3076, 3317, 3321, 3373, 3385, 3407, 3439, 3428, 3458, 3532 and processed mature forms and other forms thereof, and allelic and species variants thereof.

In some examples, such modified MMP-1 polypeptides include polypeptides having an amino acid replacement or replacements at any one or more positions corresponding to any of the following positions: 95, 100, 103, 105, 150, 151, 153, 155, 156, 159, 171, 176, 179, 180, 181, 182, 185, 187, 190, 191, 192, 194, 195, 198, 206, 207, 210, 212, 218, 223, 227, 228, 229, 230, 233, 234, 237, 240 and 259 of an unmodified MMP-1 polypeptide having a sequence of amino acids set forth in SEQ ID NO:2, or at a corresponding position in an allelic or species variant or other variant of an MMP-1 polypeptide that has at least or at least about 60%, 70%, 75%, 80%, 85%, 90%, 91%, 92%, 93%, 94%, 95%, 96%, 97%, 98%, 99% or more sequence identity to an MMP-1 polypeptide set forth in SEQ ID NO:2. For example, modified MMP-1 polypeptides provided herein include polypeptides having an amino acid modification corresponding to any one or more modifications of L95K, E100V, T103Y, D105A, D105F, D105G, D105I, D105L, D105N, D105R, D105S, D105T, D105W, R150P, D151G, S153T, F155L, F155A, D156H, D156L, D156A, D156W, D156V, D156K, D156T, D156R, G159V, G159T, G171P, A176F, D179N, E180Y, E180R, E180T, E180F, D181T, D181L, D181K, E182T, E182Q, T185R, T185Y, T185H, T185G, T185V, T185Q, T185A, T185E, N187R, N187M, N187W, N187F, N187K, N187I, N187A, E190G, Y191V, N192H, N192S, N192D, N192C, H194P, R195C, R195W, R195L, R195G, R195Q, R195A, R195D, R195V, A198G, A198L, A198M, G206A, G206S, L207R, L207V, S210V, D212G, Y218S, F223C, F223E, F223G, F223A, F223S, V227C, V227D, V227E, V227L, V227S, V227W, Q228P, L229A, L229T, L229I, A230V, D233E, I234A, I234T, I234E, I234Q, I237L, I240S, I240A, I240C, and C259Q. Such modified MMP-1 polypeptides exhibit at least 1.2 times or more activity at the permissive temperature of 25° C. compared to the non-permissive temperatures of 34° C. or 37° C., for example, 1.2, 1.3, 1.4, 1.5, 1.6, 1.7, 1.8, 1.9, 2.0, 2.5, 3.0, 3.5, 4.0, 4.5, 5, 5.5, 6, 6.5, 7, 7.5, 8, 8.5, 9, 9.5, 10, 15, 20, 30, 40, 50, 60, 70, 80, 90, 100 or more times the activity. Exemplary of such modified MMP-1 polypeptides have a sequence of amino acids set forth in any of SEQ ID NOS:6, 25, 27, 29, 31-33, 35-36, 38-39, 59, 70, 95-96, 99-101, 105, 111, 113-115, 125, 132, 148, 160, 177, 181-182, 185, 195, 200, 209, 218-219, 232-233, 235, 238-239, 241, 246, 248, 253-254, 261-264, 267, 269, 273, 275, 279, 282, 284-286, 299, 301, 305, 317, 324, 341, 354, 365, 369, 374-375, 381, 383-384, 388, 393, 397, 399, 420, 429, 436, 438, 440, 460, 466-467, 476, 483, 488, 495, 512, 524, 543, 572, 583, 607, 611, 613, 616, 620, 648, 653, 665, 678, 703, 3076 and 3532 and processed mature forms and other forms thereof, and allelic and species variants thereof.

In other examples, such modified MMP-1 polypeptides include polypeptides having an amino acid replacement or replacements at any one or more positions corresponding to any of the following positions: 95, 105, 150, 151, 155, 156, 159, 176, 179, 180, 181, 182, 185, 187, 195, 198, 206, 210, 212, 218, 223, 227, 228, 229, 230, 233, 234, 240, 259 of an unmodified MMP-1 polypeptide having a sequence of amino acids set forth in SEQ ID NO:2, or at a corresponding position in an allelic or species variant or other variant of an MMP-1 polypeptide that has at least or at least about 60%, 70%, 75%, 80%, 85%, 90%, 91%, 92%, 93%, 94%, 95%, 96%, 97%, 98%, 99% or more sequence identity to an MMP-1 polypeptide set forth in SEQ ID NO:2. For example, modified MMP-1 polypeptides provided herein include polypeptides having an amino acid modification corresponding to any one or more modifications of L95K, D105A, D105F, D105G, D105I, D105L, D105N, D105R, D105S, D105T, D105W, R150P, D151G, F155A, D156K, D156T, D156L, D156A, D156W, D156V, D156H, D156R, G159V, G159T, A176F, D179N, E180Y, E180T, E180F, D181L, D181K, E182T, E182Q, T185R, T185H, T185Q, T185A, T185E, N187R, N187M, N187F, N187K, N187I, R195V, A198L, A198M, G206A, G206S, S210V, Y218S, F223E, V227C, V227E, V227W, Q228P, L229T, L229I, D233E, I234A, I234T, I234E, I240S, I240C and C259Q. Such modified MMP-1 polypeptides exhibit at least 1.5 times or more activity at the permissive temperature of 25° C. compared to the non-permissive temperatures of 34° C. or 37° C., for example, 1.5, 1.6, 1.7, 1.8, 1.9, 2.0, 2.5, 3.0, 3.5, 4.0, 4.5, 5, 5.5, 6, 6.5, 7, 7.5, 8, 8.5, 9, 9.5, 10, 15, 20, 30, 40, 50, 60, 70, 80, 90, 100 or more times the activity. Exemplary of such modified MMP-1 polypeptides have a sequence of amino acids set forth in any of SEQ ID NOS:6, 25, 27, 29, 31-33, 35-36, 38-39, 59, 70, 96, 99-101, 105, 111, 113-115, 125, 132, 148, 160, 181-182, 185, 195, 209, 218-219, 232-233, 235, 238, 248, 253-254, 261-262, 264, 284, 301, 305, 317, 324, 341, 354, 365, 384, 388, 397, 420, 429, 436, 440, 460, 467, 476, 483, 488, 3532 and processed mature forms and other forms thereof, and allelic and species variants thereof.

In additional examples, modified MMP-1 polypeptides provided herein include modified MMP-1 polypeptides that are temperature sensitive at the permissive temperature of 25° C. and exhibit at least 30%, for example, 30%, 40%, 50%, 60%, 70%, 80%, 90%, 100%, 110%, 120%, 140%, 150% or more activity at 25° C. compared to wildtype MMP-1 at 25° C. For example, tsMMP-1 polypeptides that exhibit increased activity compared to wildtype MMP-1 include polypeptides having an amino acid replacement or replacements at any one or more positions corresponding to any of the following positions: 95, 105, 150, 156, 159, 179, 180, 182, 185, 187, 195, 198, 212, 223, 227, 234, and 240 of an unmodified MMP-1 polypeptide having a sequence of amino acids set forth in SEQ ID NO:2, or at a corresponding position in an allelic or species variant or other variant of an MMP-1 polypeptide that has at least or at least about 60%, 70%, 75%, 80%, 85%, 90%, 91%, 92%, 93%, 94%, 95%, 96%, 97%, 98%, 99% or more sequence identity to an MMP-1 polypeptide set forth in SEQ ID NO:2. For example, modified tsMMP-1 polypeptides provided herein that have increased activity at the permissive temperature of 25° C. compared to wildtype MMP-1 include polypeptides having an amino acid modification corresponding to any one or more modifications L95K, D105A, D105G, D105I, D105L, D105N, D105S, D105W, D105T, R150P, D156K, D156T, D156V, D156H, D156R, G159V, G159T, D179N, E180Y, E180T, E180F, E182T, T185H, T185Q, T185E, N187M, N187K, N187I, R195V, A198L, F223E, V227E, I234E and I240S. Exemplary of such modified MMP-1 polypeptides have a sequence of amino acids set forth in any of SEQ ID NOS:6, 27, 29, 31-32, 35-36, 38-39, 59, 99-101, 105, 113, 125, 132, 160, 181-182, 185, 219, 232-233, 238, 253, 262, 264, 284, 305, 365, 384, 460, 488 or processed mature forms and other forms thereof, and allelic and species variants thereof.

In particular, modified MMP-1 polypeptides provided herein that are temperature sensitive have an amino acid replacement or replacements at any one or more positions corresponding to any of the following positions: 95, 105, 150, 156, 159, 179, 180, 182, 185, 187, 198, 227, 234 and 240 of an unmodified MMP-1 polypeptide having a sequence of amino acids set forth in SEQ ID NO:2, or at a corresponding position in an allelic or species variant or other variant of an MMP-1 polypeptide that has at least or at least about 60%, 70%, 75%, 80%, 85%, 90%, 91%, 92%, 93%, 94%, 95%, 96%, 97%, 98%, 99% or more sequence identity to an MMP-1 polypeptide set forth in SEQ ID NO:2. Such modified MMP-1 polypeptides provided herein include polypeptides having an amino acid modification corresponding to any one or more modifications L95K, D105I, D105N, D105L, D105A, D105G, R150P, D156R, D156H, D156K, D156T, G159V, G159T, D179N, E180T, E180F, E182T, T185Q, N187I, A198L, V227E, I234E and I240S. More particularly, modified MMP-1 polypeptides provided herein include polypeptides having an amino acid modification corresponding to any one or more modifications L95K, D105N, R150P, D156K, D156T, G159V, D179N, E180T, A198L, V227E, and I240S.

Modified MMP-1 polypeptides provided herein include those that exhibit reversible or irreversible (also called non-reversible) temperature-dependent activity. In all cases, modified MMP-1 polypeptides provided herein above exhibit increased activity at a permissive temperature (e.g. 25° C.) compared to a non-permissive temperatures (e.g. 34° C. or 37° C.) For non-reversible polypeptides, exposure to the non-permissive temperature prior to, subsequently or intermittently from exposure to the permissive temperature renders the polypeptide irreversibly inactive. Thus, a modified MMP-1 polypeptide that is returned to temperature permissive conditions, for example 25° C., exhibits the same or similar activity of the MMP-1 polypeptide at non-permissive temperatures, for example, 34° C. or 37° C. For example, upon return to permissive conditions, irreversible modified MMP-1 polypeptides provided herein exhibit at or about 50%, 60%, 70%, 80%, 90%, 100%, 105%, 110%, 115%, or 120% the activity at non-permissive temperatures. Exemplary non-reversible modified MMP-1 polypeptides provided herein include polypeptides having an amino acid modification corresponding to any one or more modifications L95K, D105I, D105L, D105N, D105R, D105W, D151G, F155A, D156K, D156T, D156L, D156A, D156W, D156V, D156H, D156R, G159V, A176F, D179N, D181L, D181K, E182T, E182Q, T185R, N187F, N187I, G206A, G206S, V227C, V227E, Q228E, L229T, D233E, I234A, I234T, I234E, I240S, for example, any set forth in any of SEQ ID NOS:6, 25, 27, 35-36,38,70,96,99-101, 105, 111, 113-115, 132, 148, 160, 195, 209, 218-219, 235, 261, 264, 317, 324, 384, 388, 403, 429, 440, 460, 467, 476, 488, or processed mature forms and other forms thereof, and allelic and species variants thereof.

For reversible polypeptides, exposure to the non-permissive temperature prior to, subsequently or intermittently from exposure to the permissive temperature renders the polypeptide reversibly active. Thus, a modified MMP-1 polypeptide that is returned to temperature permissive conditions recovers activity, and thereby exhibits increased activity at the permissive temperature compared to the non-permissive temperature. In such examples, the recovered activity can be complete or partial. Thus, a modified MMP-1 polypeptide that is returned to temperature permissive conditions, for example 25° C., exhibits an increased activity compared to activity at non-permissive temperatures, for example, 34° C. or 37° C. For example, upon return to permissive conditions, reversible modified MMP-1 polypeptides provided herein exhibit at or about 120%, 125%, 130%, 140%, 150%, 160%, 170%, 180%, 200% or more of the activity at non-permissive temperatures. Exemplary reversible modified MMP-1 polypeptides provided herein include polypeptides having an amino acid modification corresponding to any one or more modifications D105A, D105F, D105G, D105S, D105T, R150P, G159T, E180Y, E180T, E180F, T185H, T185Q, T185A, T185E, N187R, N187M, N187K, R195V, A198L, A198M, S210V, Y218S, F223E, V227W, L229I and I240C, for example, any set forth in any of SEQ ID NOS: 29, 31-33, 39, 59, 125, 181-182, 185, 232-233, 238, 248, 253-254, 262, 284, 301, 305, 341, 354, 365, 397, 436, 483, or processed mature forms and other forms thereof, and allelic and species variants thereof.

2. Matrix Metalloprotease-1 Activity Mutants

Also provided herein are modified MMP-1 polypeptides that exhibit increased activity compared to wild-type MMP-1 at the permissive and non-permissive temperature. Unlike tsMMP-1 polypeptides provided herein, such activity mutants exhibit increased activity at both the permissive and non-permissive temperature compared to the MMP-1 not containing the modification (e.g. wildtype). For example, modified MMP-1s that are provided herein have increased activity compared to wildtype at a low temperature that is less then 37° C., for example, that is at or about 18° C., 19° C., 20° C., 21° C., 22° C., 23° C., 24° C., 25° C., 26° C., 27° C., 28° C., 29° C. or 30° C., in particular at or about 18° C. to 25° C., for example at or about 25° C. Modified MMP-1 s that are provided herein that have increased activity also exhibit increased activity compared to wild-type at higher temperature that is at or about 34° C., 35° C., 36° C., 37° C., 38° C. or 39° C., in particular at or about 34° C. or 37° C. The modified MMP-1s provided herein exhibit 1.1-fold, 1.2, 1.3, 1.4, 1.5, 1.6, 1.7, 1.8, 1.9, 2.0. 3.0. 4.0.5.0, 6.0, 7.0, 8.0. 9.0, 10.0, 20.0 or more increased activity than an MMP-1 not containing the modification (e.g. wildtype) at the same temperature (permissive or non-permissive). For example, the modified MMP-1s provided herein exhibit 110%, 120%, 130%, 140%, 150%, 160%, 170%, 180%, 190%, 200%, 250%, 300%, 400%, 500%, 600%, 700%, 800%, 900%, 1000% or more increased activity than an MMP-1 not containing the modification (e.g. wildtype) at the same temperature (permissive or non-permissive).

Typically, the modification is an amino acid replacement. The amino acid replacement or replacements can be at any one or more positions corresponding to any of the following positions: 81, 84, 85, 86, 87, 89, 104, 105, 106, 107, 108, 109, 124, 131, 133, 134, 135, 143, 146, 147, 150, 152, 153, 154, 157, 158, 160, 161, 164, 166, 167, 180, 183, 189, 190, 207, 208, 211, 213, 214, 216, 218, 220, 222, 223, 224, 225, 226, 227, 228, 229, 230, 231, 232, 235, 236, 238, 239, 244, 249, 254, 256, 257, 258 of an unmodified MMP-1 polypeptide having a sequence of amino acids set forth in SEQ ID NO:2, or at a corresponding position in an allelic or species variant or other variant of an MMP-1 polypeptide that has at least or at least about 60%, 70%, 75%, 80%, 85%, 90%, 91%, 92%, 93%, 94%, 95%, 96%, 97%, 98%, 99% or more sequence identity to an MMP-1 polypeptide set forth in SEQ ID NO:2. Amino acid replacements include replacement of amino acids to an acidic (D or E); basic (H. K or R); neutral (C, N, Q, T, Y, S, G) or hydrophobic (F, M, W, I V, L A, P) amino acid residue. For example, amino acid replacements at the noted positions include replacement by amino acid residues E, H, R, C, Q, T, S, G, M, W, I, V, L, A, P, N, F, D, Y or K.

For example, modified MMP-1 polypeptides provided herein can include polypeptides having an amino acid modification corresponding to any one or more modifications of F81 L (i.e. replacement of F by L at a position corresponding to position 81 of an MMP-1 polypeptide set forth in SEQ ID NO:2), F81A, F81G, F81Q, F81R, F81H, T84H, T84L, T84D, T84R, T84G, T84A, E85S, E85V, G86S, N87P, N87R, N87G, N87Q, R89A, R89T, R89G, R89K, P104E, P104D, P104Q, D105V, L106V, P107T, P107S, P107A, R108E, R108A, R108K, R108S, A109S, A109R, A109G, A109M, A109V, N124G, T131D, K132R, V133T, V133L, S134E, S134D, E135M, S143I, R146S, G147R, G147F, R150E, R150G, R150M, T150T, R150A, R150N, R150K, R150L, R150V, R150D, N152G, N152F, N152L, N152I, S153T, S153P, S153F, S153D, S153Y, P154S, P154I, G157F, P158V, P158I, G160Q, N161L, N161R, N161Y, N161E, N161T, N161I, N161V, N161F, N161Q, H164S, F166W, Q167R, Q167A, Q167S, Q167F, Q167P, Q167T, Q167V, Q167M, E180D, R183S, R189N, R189T, R189Q, E190D, L207M, S208K, S208R, S208L, T211N, I213G, G214L, G214E, L216I, Y218W, S220R, S220A, S220Q, S220T, S220G, S220M, S220V, S220N, T222R, T222P, T222S, T222F, T222N, F223Y, F223H, 2224Q, S224K, S224D, G225Q, G225E, G225H, D226S, D226E, D226P, D226I, V227T, Q228A, Q228D, Q228E, Q228G, Q228H, Q228K, Q228L, Q228M, Q228N, Q228R, Q228S, Q228T, Q228W, Q228Y, L229Q, L229P, L229V, A230G, A230W, A230D, A230I, A230S, A230C, A230V, A230T, A230M, A230N, A230H, Q231I, Q231A, Q231F, Q231D, Q231G, Q231V, Q231W, Q231S, Q231H, Q231M, D232H, D232G, D232R, D232P, D232Y, D232S, D232F, D232V, D232K, D232W, D232Q, D232E, D232T, D232L, D235G, D235A, D235L, D235E, D235R, D235Q, D235T, D235N, G236M, G236R, G236S, G236T, G236C, G236K, G236E, G236L, G236N, Q238T, A239S, A239V, A239L, A239I, A239G, A239K, A239H, A239R, S244W, S244Q, Q249W, Q254S, P256S, K257E, K257R, or A258P.

In particular, modified MMP-1 polypeptides provided herein having increased activity have an amino acid modification corresponding to any one or more modifications of N161I, S208K, I213G, G214E, Q228A, Q228D, Q228E, Q228G, Q228H, Q228K, Q228L, Q228M, Q228N, Q228R, Q228S, Q228W, Q228Y, L229V, A230G, A230D, A230S, A230C, A230T, A230M, A230N, A230H, Q231A, Q231D, Q231G, Q231V, Q231S, D232H, D232G, D232P, D232V, D232K, D232W, D232Q, D232E, or D232T. In one example, activity mutants of MMP-1 provided herein including modified MMP-1 polypeptides having one of more modifications of S208K, I213G, or G214E.

Exemplary modified MMP-1 polypeptides have a sequence of amino acids set forth in any of SEQ ID NOS: 37, 41, 42, 44, 46, 48, 51, 53, 56, 57, 58, 174, 358, 366, 373, 391, 402, 403, 404, 405, 406, 408, 409, 410, 411, 412, 414, 415, 418, 419, 428, 437, 439, 535, 543, 544, 546, 553, 573, 662, 687, 689, 692, 693, 695, 697, 698, 700, 701, 702, 703, 781, 783, 786, 795, 796, 790, 838, 836, 840, 852, 846, 853, 864, 870, 884, 911, 897, 903, 899, 938, 941, 948, 934, 1160, 1159, 1166, 1194, 1205, 1207, 1215, 1217, 1219, 1225, 1233, 1239, 1245, 1246, 1248, 1251, 1530, 1653, 1675, 1699, 1707, 1710, 1711, 1741, 1895, 1947, 1961, 1968, 2024, 2025, 2028, 2030, 2043, 2048, 2087, 2088, 2098, 2111, 2114, 2116, 2117, 2118, 2124, 2125, 2121, 2126, 2176, 2218, 2228, 2241, 2231, 2233, 2235, 2236, 2239, 2242, 2423, 2495, 2496, 2497, 2702, 2703, 2715, 2743 2767, 2776, 2791, 2828, 2874, 2887, 2876, 2877, 2878, 2880, 2882, 2885, 2912, 2914, 2917, 2919, 2926, 2927, 2930, 2934, 2947, 2948, 2953, 2965, 2974, 2979, 2983, 2984, 2986, 2993, 2994, 2995, 2996, 2997, 2998, 2999, 3001, 3003, 3004, 3005, 3006, 3009, 3010, 3011, 3012, 3013, 3014, 3016, 3018, 3019, 3021, 3022, 3025, 3027, 3028, 3029, 3032, 3038, 3039, 3042, 3044, 3046, 3047, 3049, 3051, 3057, 3086, 3100, 3101, 3102, 3108, 3109, 3113, 3114, 3115, 3181, 3187, 3282, 3373, 3412, 3422, 3424, or 3458 and processed mature forms and other forms thereof, and allelic and species variants thereof.

3. Combinations

Provided herein are modified MMP-1 polypeptides that contain 2, 3, 4, 5, 6, 7, 8, 9, 10, 11, 12, 13, 14, 15, 16, 17, 18, 19, 20 or more modifications compared to a starting or reference MMP-1 polypeptide. Modified MMP-1 polypeptides provided herein can contain any two or more modifications provided above. The two or more modifications can include two or more temperature-sensitive modifications, two or more activity modifications, or at least one temperature sensitive modification and at least one activity modification.

For example, modified MMP-1 polypeptides provided herein contain amino acid replacements at any two or more positions corresponding to any of the following positions: 84, 85, 95, 98, 99, 100, 103, 104, 105, 106, 109, 110, 111, 112, 118, 123, 124, 126, 147, 150, 151, 152, 153, 155, 156, 158, 159, 170, 171, 176, 178, 179, 180, 181, 182, 183, 185, 187, 188, 189, 190, 191, 192, 194, 195, 197, 198, 206, 207, 208, 210, 211, 212, 218, 223, 227, 228, 229, 230, 233, 234, 237, 240, 251, 254, 255, 256, 257, 258 or 259 of an unmodified MMP-1 polypeptide having a sequence of amino acids set forth in SEQ ID NO:2, or at a corresponding position in an allelic or species variant or other variant of an MMP-1 polypeptide that has at least or at least about 60%, 70%, 75%, 80%, 85%, 90%, 91%, 92%, 93%, 94%, 95%, 96%, 97%, 98%, 99% or more sequence identity to an MMP-1 polypeptide set forth in SEQ ID NO:2 Generally, such combination mutants are temperature sensitive and exhibit increased enzymatic activity at a permissive temperature compared with activity of the tsMMP-1 polypeptide at a non-permissive temperature. Typically, combination mutants also retain activity at the permissive temperature compared to the single mutant MMP-1 polypeptides alone or compared to an unmodified MMP-1 polypeptide not containing the amino acid changes (e.g. a wildtype MMP-1 polypeptide set forth in SEQ ID NO:2 or active forms or other forms thereof) at the permissive or non-permissive temperature.

Exemplary MMP-1 combination mutants provided herein contain amino acid replacements at any two or more positions corresponding any of the following positions: 95, 105, 150, 156, 159, 179, 180, 182, 185, 187, 198, 227, 234 and 240 of an unmodified MMP-1 polypeptide having a sequence of amino acids set forth in SEQ ID NO:2, or at a corresponding position in an allelic or species variant or other variant of an MMP-1 polypeptide that has at least or at least about 60%, 70%, 75%, 80%, 85%, 90%, 91%, 92%, 93%, 94%, 95%, 96%, 97%, 98%, 99% or more sequence identity to an MMP-1 polypeptide set forth in SEQ ID NO:2. For example, modified MMP-1 polypeptides provided herein include polypeptides having amino acid modification corresponding to any two or more modifications L95K, D105I, D105N, D105L, D105A, D105G, R150P, D156R, D156H, D156K, D156T, G159V, G159T, D179N, E180T, E180F, E182T, T185Q, N187I, A198L, V227E, I234E and I240S. More particularly, modified MMP-1 polypeptides provided herein include polypeptides having amino acid modification corresponding to any two or more modifications L95K, D105N, R150P, D156K, D156T, G159V, D179N, E180T, A198L, V227E, and I240S. It is understood that at least two different positions are modified in the combination mutants provided herein. Exemplary MMP-1 combination mutant polypeptides provided herein are set forth in Table 15 in Example 3. For example, combination mutants provided herein that exhibit temperature sensitivity include D156K/G159V/D179N; R150P/V227E; D156T/V227E; G159V/A198L; D105N/A198L; D179N/V227E; A198L/V227E; E180T/V227E; D179N/A198L; D156K/D179N; D105N/R150P/D156K/G159V/D179N/E180T; D105N/R150P/E180T; G159V/I240S; D156T/D179N/I240S; D156T/G159V; R150P/E180T; D156T/D179N; D179N/I240S; L95K D156T/D179N; G159V/D179N; L95K/D105N/E180T; R150P/D156T/A198L; L95K/D105N/R150P/D156T/G159V/A198L/V227E/I240S; L95K/R150P; or D105N/E180T. Exemplary modified MMP-1 polypeptides have a sequence of amino acids set forth in any of SEQ ID NOS: 3507-3531 and processed mature forms and other forms thereof, and allelic and species variants thereof.

Combination mutants provided herein also can include amino acid modification C259Q and at least one other modification. The other modification can be another temperature sensitive modification, for example, any of modifications L95K, D105I, D105N, D105L, D105A, D105G, R150P, D156R, D156H, D156K, D156T, G159V, G159T, D179N, E180T, E180F, E182T, T185Q, N187I, A198L, V227E, I234E and I240S. Exemplary of such combination mutants include C259Q/D105N; C259Q/R150P; C259Q/G159V; C259Q/D179N/ or C259Q/E180T, for example, as set forth in SEQ ID NOS: 3533-3537.

Also included among the combination mutants provided herein are MMP-1 polypeptides that contain at least one temperature sensitive modification and at least one activity modification, and retain temperature sensitivity. For example, such combination mutants exhibit increased activity at a permissive temperature compared to a non-permissive temperature as described herein above. Any one or more of the temperature sensitive mutants provided in Section D.1 above can be combined with any one or more of the activity mutants provided in Section D.2 above. For example, a combination mutant provided herein contains at least one modification of L95K, D105I, D105N, D105L, D105A, D105G, R150P, D156R, D156H, D156K, D156T, G159V, G159T, D179N, E180T, E180F, E182T, T185Q, N187I, A198L, V227E, I234E and I240S and at least one modification of N161I, S208K, I213G, G214E, Q228A, Q228D, Q228E, Q228G, Q228H, Q228K, Q228L, Q228M, Q228N, Q228R, Q228S, Q228W, Q228Y, L229V, A230G, A230D, A230S, A230C, A230T, A230M, A230N, A230H, Q231A, Q231D, Q231G, Q231V, Q231S, D232H, D232G, D232P, D232V, D232K, D232W, D232Q, D232E, or D232T. For example, a combination mutant provided herein contains at least one modification of L95K, D105N, R150P, D156K, D156T, G159V, D179N, E180T, A198L, V227E, or I240S and at least one modification of S208K, I213G, or G214E. Exemplary combination mutants provided herein include S208K/G159V; S208K/D179N; S208K/V227E; G214E/G159V; G214E/D179N; or I213G/D179N, for example, as set forth in any of SEQ ID NOS: 3541-3546.

4. Additional Modifications

Any modified MMP-1 polypeptide provided herein also can contain one or more other modifications described in the art. The additional modifications can include, for example, any amino acid substitution, deletion or insertion known in the art. In addition to containing one or more modification(s) described above in Sections D.1 and D.2, any modified MMP-1 polypeptide provided herein can contain 1, 2, 3, 4, 5, 6, 7, 8, 9, 10, 11, 12, 13, 14, 15, 16, 17, 18, 19, 20 or more additional modifications. Typically, MMP-1 polypeptides retain enzymatic activity of wildtype MMP-1 at the permissive or non-permissive temperature, or exhibit increased enzymatic activity of wildtype MMP-1. Generally, where at least one modification is a temperature sensitive mutation, the MMP-1 polypeptide also exhibits increased activity at the permissive temperature (e.g. 25° C.) compared to the non-permissive temperature (e.g. 34° C. or 37° C.). The additional modifications can confer additional properties to the enzyme, for example, increased stability, increased half-life and/or increased resistance to inhibitors, for example, TIMP. The additional modifications include modifications to the primary sequence of the polypeptide, as well as other modification such as PEGylation and glycosylation of the polypeptide. Generally, such polypeptides include one or more modifications provided herein and exhibit increased activity at the lower temperature then at the higher temperature. For example, any of the amino acid replacements, including allelic variants and other variants known in the art, as set forth in SEQ ID NO:3506 or 3549, can be included herein. Exemplary modifications that can be included in a polypeptide provided herein include, but are not limited to, modifications T4P, Q10P, R30M, R30S, T96R, A114V, F166C, I172V, D181H, R189T, H199A; E200A, G214E, D232N, D233G, R243S, Q254P, I271A, R272A, T286A, I298T, E314G, F315S, V374M, R386Q, S387T, G391S, and T432A of a polypeptide set forth in SEQ ID NO:2.

5. Other MMPs

Matrix metalloproteases are highly homologous polypeptides and exhibit similar specificities for extracellular matrix components. Exemplary sequences of MMPs are set forth in Table 5, for example, any set forth in SEQ ID NOS:1, 711, 714, 717, 720, 723, 726, 729, 732, 735, 738, 741, 744, 747, 750, 753, 756, 759, 762, 765, 768, 771, 774 or 777 or zymogen forms, processed mature forms or other forms thereof, or allelic or species variants thereof. FIG. 1 provides an alignment of the zymogen form of exemplary MMP polypeptides. Thus, any of the modifications provided herein in an MMP-1 can be made in any other MMP polypeptide. Hence, based on the description herein, any MMP, species, allelic variant or other variant, can be made temporally active (reversible or irreversible) by virtue of activity at a permissive temperature (generally a lower temperature) compared to a nonpermissive temperature (generally a higher temperature). Such tsMMP mutants can be used by one of skill in the art and used in compositions, processes or methods for the treatment of ECM-mediated diseases or conditions.

It is within the level of one of skill in the art to align various MMPs to MMP-1 (for example set forth in SEQ ID NO:2) and identify corresponding residues. Any of the modifications provided herein can be made in any other MMP at the corresponding residue. One of skill in the art can test the activity of the resulting modified polypeptide for enzymatic activity and/or temperature sensitivity at a permissive temperature compared to a non-permissive temperature. In particular, it is understood that conservative amino acid differences at a corresponding position in an MMP are functionally invariant. Thus, where a residue in MMP-1 aligns with a conservative residue thereto in another MMP, it is understood that such a residue is contemplated for modification herein. For example, position 95 in an MMP-1 set forth in SEQ ID NO:2 is a leucine (L). Alignment of SEQ ID NO:2 with other MMPs shows that position 95 in other MMPs is a leucine, isoleucine (I) or valine (V) residue (see FIG. 1). Each of L, I and V are conservative residues.

In particular, provided herein are modified MMP polypeptides that are modified by one or more amino acid replacement to confer temperature sensitivity and/or increased activity by effecting a corresponding MMP-1 modification at a corresponding residue Exemplary modifications provided herein include modification of any MMP, for example, an MMP-8, MMP-13, MMP-18, MMP-2, MMP-9, MMP-3, MMP-10, MMP-11, MMP-7, MMP-26 and MMP-12, at any one or more positions corresponding to any of the following positions: 95, 105, 151, 156, 159, 176, 179, 180, 181, 182, 185, 195, 198, 206, 210, 212, 218, 223, 228, 229, 233, 234, and 240 of an unmodified MMP-1 polypeptide having a sequence of amino acids set forth in SEQ ID NO:2. In other example, exemplary modifications provided herein include modification of any MMP, for example, an MMP-8, MMP-13, MMP-18, MMP-2, MMP-9, MMP-3, MMP-10, MMP-11, MMP-7, MMP-26 and MMP-12, at any one or more positions corresponding to any of the following positions: 81, 89, 109, 131, 133, 154, 157, 158, 160, 164, 166, 180, 207, 216, 218, 223, 228, 229, 231, 232, 236, 238, 256. The modification includes any one or more of the modifications provided herein in sections D.1 and D.2 at the corresponding position to the recited position in MMP-1. For example, residue 95 in an MMP-1 polypeptide set forth in SEQ ID NO:2 corresponds to residue 113 in an MMP-8 polypeptide set forth in SEQ ID NO:711. Thus, provided herein are modified MMP-8 polypeptides having an amino acid modification L113K of an unmodified MMP-8 polypeptide having a sequence of amino acids set forth in SEQ ID NO:711. Similar modifications are provided herein based on this description.

Any modified MMP polypeptide provided herein also can contain one or more other modifications described in the art. The additional modifications can include, for example, any amino acid substitution, deletion or insertion known in the art. In addition to containing one or modification described above in Sections D.1 and D.2, any modified MMP polypeptide provided herein can contain 1, 2, 3, 4, 5, 6, 7, 8, 9, 10, 11, 12, 13, 14, 15, 16, 17, 18, 19, 20 or more additional modifications, so long as the resulting MMP polypeptides exhibits increased activity at the permissive temperature (e.g. 25° C.) compared to the non-permissive temperature (e.g. 34° C. or 37° C.) and retains activity of wildtype MMP at the permissive or non-permissive temperature. The additional modifications can confer additional properties to the enzyme, for example, increased stability, increased half-life and/or increased resistance to inhibitors, for example, TIMP. The additional modifications include modifications to the primary sequence of the polypeptide, as well as other modification such as PEGylation and glycosylation of the polypeptide. Generally, such polypeptides include one or more modifications provided herein and exhibit increased activity at the lower temperature then a higher temperature. Exemplary modifications that can be included in a polypeptide provided herein include, but are not limited to, any modifications set forth in Table 6, below.

TABLE 6
Exemplary modifications in MMPs

| | SEQ | |
|---|---|---|
| MMP | ID NO | Amino Acid Modifications |
| MMP-8 | 711 | S3C; T32I; K87E; E153G; D193V; S229T; |
| | | N246Y; L249V; Q251A; Q251D; Q251G; |
| | | Q251V; Q251S; D252H; D252G; D252P; |
| | | D252V; D252K; D252W; D252Q; D252E; |
| | | D252T; K460T |
| MMP-13 | 714 | H2L; A8V; F75S; D89H; L254V; D257H; |
| | | D257G; D257P; D257V; D257K; D257W; |
| | | D257Q; D257E; D257T; D390G; 1427T |
| MMP-2 | 720 | A27S; R101H; D210Y; A228T; F239L; E404K; |
| | | L433V; Q435A;Q435D; Q435G; Q435V; |
| | | Q435S; D436H; D436G; D436P; D436V; |
| | | D436K; D436W; D436Q; D436E; |
| | | D436T; A447V; T498M; V620I; V621L; S6441 |
| MMP-9 | 723 | A20V; N38S; E82K; N127K; L187F; R239H; |
| | | T258I; Q279R; L431V; D434H; D434G; D434P; |
| | | D434V; D434K; D434W; D434Q; D434E; |
| | | D434T; F571V; P574R; R668Q |
| MMP-3 | 726 | K45E; H113P; R248W; L251V; Q253A; Q253D; |
| | | Q253G; Q253V; Q253S; D254H; D254G; |
| | | D254P; D254V; D254K; D254W; D254Q; |
| | | D254E; D254T |
| MMP-10 | 729 | L4V; V8G; R53K; G65R; E142Q; L250V; |
| | | Q252A; Q252D; Q252G; Q252V; Q252S; |
| | | D253H; D253G; D253P; D253V; D253K; |
| | | D253W; D253Q; D253E; D253T; F226L; |
| | | G282E; L440F; H475L |
| MMP-11 | 732 | V38A; E44K; P6IL; S86P; D166N; F182S; |
| | | L245V; D248H; D248G; D248P; D248V; |
| | | D248K; D248W; D248Q; D248E; D248T; |
| | | Q323H |
| MMP-7 | 735 | C7W; R77R; S115T; G137D; P241L; L246V; |
| | | Q248A; Q248D; Q248G; Q248V; Q248S; |
| | | D249H; D249G; D249P; D249V; D249K; |
| | | D249W; D249Q; D249E; D249T |
| MMP-26 | 738 | K43E; S46L; Q239A; Q239D; Q239E; Q239G; |
| | | Q239H; Q239K; Q239L; Q239M; Q239N; |
| | | Q239R; Q239S; Q239W; Q239Y; Q239V; |
| | | L240V; D243H; D243G; D243P; D243V; |
| | | D243K; D243W; D243Q; D243E; D243T; |
| | | I260M |
| MMP-12 | 741 | L250V; D253H; D253G; D253P; D253V; |
| | | D253K; D253W; D253Q; D253E; D253T; |
| | | N357S; F468L; G469R |
| MMP-19 | 765 | R103C; L243V; D246H; D246G; D246P; |
| | | D246V; D246K; D246W; D246Q; D246E; |
| | | D246T; P245S; P488T; T491M |

### E. METHODS OF PRODUCING NUCLEIC ACIDS ENCODING tsMMPS AND POLYPEPTIDES THEREOF

Modified MMP polypeptides, for example tsMMPs set forth herein, can be obtained by methods well known in the art for protein purification and recombinant protein expression. Any method known to those of skill in the art for identification of nucleic acids that encode desired genes can be used. Any method available in the art can be used to obtain a full length (i.e., encompassing the entire coding region) cDNA or genomic DNA clone encoding a desired MMP, such as from a cell or tissue source. Modified or variant tsMMPs, can be engineered from a wildtype polypeptide, such as by site-directed mutagenesis.

Polypeptides can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding a desired polypeptide, including for example, polymerase chain reaction (PCR) methods. A nucleic acid containing material can be used as a starting material from which a desired polypeptide-encoding nucleic acid molecule can be isolated. For example, DNA and mRNA preparations, cell extracts, tissue extracts, fluid samples (e.g. blood, serum, saliva), samples from healthy and/or diseased subjects can be used in amplification methods. Nucleic acid libraries also can be used as a source of starting material. Primers can be designed to amplify a desired polypeptide. For example, primers can be designed based on expressed sequences from which a desired polypeptide is generated. Primers can be designed based on back-translation of a polypeptide amino acid sequence. Nucleic acid molecules generated by amplification can be sequenced and confirmed to encode a desired polypeptide.

Additional nucleotide sequences can be joined to a polypeptide-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to a polypeptide-encoding nucleic acid molecule. Examples of such sequences include, but are not limited to, promoter sequences designed to facilitate intracellular protein expression, and secretion sequences, for example heterologous signal sequences, designed to facilitate protein secretion. Such sequences are known to those of skill in the art. For example, exemplary heterologous signal sequences include, but are not limited to, human kappa IgG heterologous signal sequence set forth in SEQ ID NO:3468. For bacterial expression, and exemplary heterologous signal sequence is the pelB leader sequence, for example, as set forth in SEQ ID NO: 3547. Additional nucleotide residues sequences such as sequences of bases specifying protein binding regions also can be linked to enzyme-encoding nucleic acid molecules. Such regions include, but are not limited to, sequences of residues that facilitate or encode proteins that facilitate uptake of an enzyme into specific target cells, or otherwise alter pharmacokinetics of a product of a synthetic gene. For example, enzymes can be linked to PEG moieties.

In addition, tags or other moieties can be added, for example, to aid in detection or affinity purification of the polypeptide. For example, additional nucleotide residues sequences such as sequences of bases specifying an epitope tag or other detectable marker also can be linked to enzyme-encoding nucleic acid molecules. Exemplary of such sequences include nucleic acid sequences encoding a His tag (e.g., 6×His, HHHHHH; SEQ ID NO:3465) or Flag Tag (DYKDDDDK; SEQ ID NO:3467).

The identified and isolated nucleic acids can then be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasmids such as pCMV4, pBR322 or pUC plasmid derivatives or the Bluescript vector (Stratagene, La Jolla, Calif.). Other expression vectors include the pET303CTHis (SEQ ID NO:3466; Invitrogen, CA) or pET-26B vector (SEQ ID NO:3548) expression vector exemplified herein. The insertion into a cloning vector can, for example, be accomplished by ligating the DNA fragment into a cloning vector which has complementary cohesive termini. Insertion can be effected using TOPO cloning vectors (INVITROGEN, Carlsbad, Calif.). If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized oligonucleotides encoding restriction endonuclease recognition sequences. In an alternative method, the cleaved vector and protein gene can be modified by homopolymeric tailing. Recombinant molecules can be introduced into host cells via, for example, transformation, transfection, infection, electroporation and sonoporation, so that many copies of the gene sequence are generated.

In specific embodiments, transformation of host cells with recombinant DNA molecules that incorporate the isolated protein gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA.

1. Vectors and Cells

For recombinant expression of one or more of the desired proteins, such as any described herein, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals also can be supplied by the native promoter for enzyme genes, and/or their flanking regions.

Also provided are vectors that contain a nucleic acid encoding the enzyme. Cells containing the vectors also are provided. The cells include eukaryotic and prokaryotic cells, and the vectors are any suitable for use therein.

Prokaryotic and eukaryotic cells, including endothelial cells, containing the vectors are provided. Such cells include bacterial cells, yeast cells, fungal cells, Archea, plant cells, insect cells and animal cells. The cells are used to produce a protein thereof by growing the above-described cells under conditions whereby the encoded protein is expressed by the cell, and recovering the expressed protein. For purposes herein, for example, the enzyme can be secreted into the medium.

Provided are vectors that contain a sequence of nucleotides that encodes the proenzyme polypeptide coupled to the native or heterologous signal sequence, as well as multiple copies thereof. The vectors can be selected for expression of the enzyme protein in the cell or such that the enzyme protein is expressed as a secreted protein. The proenzyme (i.e. zymogen) form of the enzyme can be purified for use as an activatable, i.e. conditional active, enzyme herein. Alternatively, upon secretion the prosegment can be cleaved by chemical agents or catalytically or autocatalytically to generate a mature enzyme by the use of a processing agent. This processing step can be performed during the purification step and/or immediately before use of the enzyme. If desired, the processing agent can be dialyzed away or otherwise purified away from the purified protein before use. Alternative or additionally, if necessary, the enzyme can be purified such that the prosegment is removed from the preparation.

A variety of host-vector systems can be used to express the protein coding sequence. These include but are not limited to mammalian cell systems transfected with plasmid DNA or infected with virus (e.g. vaccinia virus, adenovirus and other viruses); insect cell systems infected with virus (e.g. baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination). Expression of nucleic acid sequences encoding protein, or domains, derivatives, fragments or homologs thereof, can be regulated by a second nucleic acid sequence so that the genes or fragments thereof are expressed in a host transformed with the recombinant DNA molecule(s). For example, expression of the proteins can be controlled by any promoter/enhancer known in the art. In a specific embodiment, the promoter is not native to the genes for a desired protein. Promoters which can be used include but are not limited to the SV40 early promoter (Bernoist and Chambon, Nature 290:304-310 (1981)), the promoter contained in the 3′ long terminal repeat of Rous sarcoma virus (Yamamoto et al. Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al., Nature 296:39-42 (1982)); prokaryotic expression vectors such as the β-lactamase promoter (Jay et al., (1981) Proc. Nail. Acad. Sci. USA 78:5543) or the tac promoter (DeBoer et al., Proc. Natl. Acad. Sci. USA 80:21-25 (1983)); see also “Useful Proteins from Recombinant Bacteria”: in Scientific American 242:79-94 (1980)); plant expression vectors containing the nopaline synthase promoter (Herrara-Estrella et al., Nature 303:209-213 (1984)) or the cauliflower mosaic virus 35S RNA promoter (Gardner et al., Nucleic Acids Res. 9:2871 (1981)), and the promoter of the photosynthetic enzyme ribulose bisphosphate carboxylase (Herrera-Estrella et al., Nature 310:115-120 (1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 38:639-646 (1984); Omnitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 315:115-122 (1985)), immunoglobulin gene control region which is active in lymphoid cells (Grosschedl et al., Cell 38:647-658 (1984); Adams et al., Nature 318:533-538 (1985); Alexander et al., Mol. Cell Biol. 7:1436-1444 (1987)), mouse mammary tumor virus control region which is active in testicular, breast, lymphoid and mast cells (Leder et al., Cell 45:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-1648 (1985); Hammer et al., Science 235:53-58 1987)), alpha-1 antitrypsin gene control region which is active in liver (Kelsey et al., Genes and Devel. 1:161-171 (1987)), beta globin gene control region which is active in mycloid cells (Magram et al., Nature 315:338-340 (1985); Kollias et al., Cell 46:89-94 (1986)), myelin basic protein gene control region which is active in oligodendrocyte cells of the brain (Readhead et al., Cell 48:703-712 (1987)), myosin light chain-2 gene control region which is active in skeletal muscle (Shani, Nature 314:283-286 (1985)), and gonadotrophic releasing hormone gene control region which is active in gonadotrophs of the hypothalamus (Mason et al., Science 234:1372-1378 (1986)).

In a specific embodiment, a vector is used that contains a promoter operably linked to nucleic acids encoding a desired protein, or a domain, fragment, derivative or homolog, thereof, one or more origins of replication, and optionally, one or more selectable markers (e.g., an antibiotic resistance gene). Exemplary plasmid vectors for transformation of E. coli cells, include, for example, the pQE expression vectors (available from Qiagen, Valencia, Calif.; see also literature published by Qiagen describing the system). pQE vectors have a phage T5 promoter (recognized by E. coli RNA polymerase) and a double lac operator repression module to provide tightly regulated, high-level expression of recombinant proteins in E. coli, a synthetic ribosomal binding site (RBS II) for efficient translation, a 6×His tag coding sequence, t₀ and T1 transcriptional terminators, ColE1 origin of replication, and a beta-lactamase gene for conferring ampicillin resistance. The pQE vectors enable placement of a 6×His tag at either the N- or C-terminus of the recombinant protein. Such plasmids include pQE 32, pQE 30, and pQE 31 which provide multiple cloning sites for all three reading frames and provide for the expression of N-terminally 6×His-tagged proteins. Other exemplary plasmid vectors for transformation of E. coli cells, include, for example, the pET expression vectors (see, U.S. Pat. No. 4,952,496; available from NOVAGEN, Madison, Wis.; see, also literature published by Novagen describing the system). Such plasmids include pET 11a, which contains the T7lac promoter, T7 terminator, the inducible E. coli lac operator, and the lac repressor gene; pET 12a-c, which contains the T7 promoter, T7 terminator, and the E. coli ompT secretion signal; and pET 15b and pET19b (OVAGEN, Madison, Wis.), which contain a His-Tag™ leader sequence for use in purification with a His column and a thrombin cleavage site that permits cleavage following purification over the column, the T7-lac promoter region and the T7 terminator, and pET-26B (SEQ ID NO:3548). An additional pET vector is pET303CTHis (set forth in SEQ ID NO: 3466; Invitrogen, CA), which cont ins a T7lac promoter, T7 terminator, the inducible E. coli lac operator, a beta-lactamase gene for conferring ampicillin resistance, and also a His-Tag sequence for use in purification.

Exemplary of a vector for mammalian cell expression is the HZ24 expression vector. The HZ24 expression vector was derived from the pCI vector backbone (Promega). It contains DNA encoding the Beta-lactamase resistance gene (AmpR), an F1 origin of replication, a Cytomegalovirus immediate-early enhancer/promoter region (CMV), and an SV40 late polyadenylation signal (SV40). The expression vector also has an internal ribosome entry site (IRES) from the ECMV virus (Clontech) and the mouse dihydrofolate reductase (DHFR) gene.

2. Expression

Modified MMP polypeptides, for example tsMMPs, can be produced by any method known to those of skill in the art including in vivo and in vitro methods. Desired proteins can be expressed in any organism suitable to produce the required amounts and forms of the proteins, such as for example, needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Many expression vectors are available and known to those of skill in the art and can be used for expression of proteins. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

Modified MMP polypeptides, for example tsMMPs, also can be utilized or expressed as protein fusions. For example, an enzyme fusion can be generated to add additional functionality to an enzyme. Examples of enzyme fusion proteins include, but are not limited to, fusions of a signal sequence, a tag such as for localization, e.g. a his₆ tag or a myc tag, or a tag for purification, for example, a GST fusion, and a sequence for directing protein secretion and/or membrane association.

Generally, modified MMP polypeptides, for example tsMMPs, are expressed in an inactive zymogen form. Zymogen conversion can be achieved by exposure to chemical agents, to other proteases or to autocatalysis to generate a mature enzyme as described elsewhere herein. Any form of an enzyme is contemplated herein. It is understood that, if provided and expressed in a zymogen form, that it is activated prior to use by a processing agent.

a. Prokaryotic Cells

Prokaryotes, especially E. coli, provide a system for producing large amounts of proteins. Transformation of E. coli is simple and rapid technique well known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated kPL promoter.

Proteins, such as any provided herein, can be expressed in the cytoplasmic environment of E. coli. The cytoplasm is a reducing environment and for some molecules, this can result in the formation of insoluble inclusion bodies. Reducing agents such as dithiothreotol and β-mercaptoethanol and denaturants, such as guanidine-HCl and urea can be used to resolubilize the proteins. An alternative approach is the expression of proteins in the periplasmic space of bacteria which provides an oxidizing environment and chaperonin-like and disulfide isomerases and can lead to the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader (SEQ ID NO: 3547) from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast Cells

Yeasts such as Saccharomyces cerevisae, Schizosaccharomyces pombe, Yarrowia lipolytica, Kluyveromyces lactis and Pichia pastoris are well known yeast expression hosts that can be used for production of proteins, such as any described herein. Yeast can be transformed with episomal replicating vectors or by stable chromosomal integration by homologous recombination. Typically, inducible promoters are used to regulate gene expression. Examples of such promoters include GAL1, GAL7 and GAL5 and metallothionein promoters, such as CUP1, AOX1 or other Pichia or other yeast promoter. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

c. Insect Cells

Insect cells, particularly using baculovirus expression, are useful for expressing polypeptides such as matrix-degrading enzymes. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the bombyx mori nuclear polyhedrosis virus (BmNPV) and an insect cell line such as Sf9 derived from Spodoptera frugiperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express proteins including tsMMPs. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. IRES elements also can be added to permit bicistronic expression with another gene, such as a selectable marker. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase (DHFR) and thymidine kinase. For example, expression can be performed in the presence of methotrexate to select for only those cells expressing the DHFR gene. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NS0 (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. Examples include CHO-S cells (Invitrogen, Carlsbad, Calif., cat #11619-012) and the serum free EBNA-1 cell line (Pham et al., (2003) Biotechnol. Bioeng. 84:332-42.). Cell lines also are available that are adapted to grow in special mediums optimized for maximal expression. For example, DG44 CHO cells are adapted to grow in suspension culture in a chemically defined, animal product-free medium.

e. Plants

Transgenic plant cells and plants can be used to express proteins such as any described herein. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline synthase promoter, the ribose bisphosphate carboxylase promoter and the ubiquitin and UBQ3 promoters.

Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce matrix-degrading enzymes. Because plants have different glycosylation patterns than mammalian cells, this can influence the choice of protein produced in these hosts.

3. Purification Techniques

Method for purification of polypeptides, including modified MMP polypeptides such as tsMMPs or other proteins, from host cells will depend on the chosen host cells and expression systems. For secreted molecules, proteins are generally purified from the culture media after removing the cells. For intracellular expression, cells can be lysed and the proteins purified from the extract. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary, the proteins can be extracted and further purified using standard methods in the art. If there are free cysteines, these can be replaced with other amino acids, such as serine. Replacement of free cysteines can prevent unwanted aggregation.

Generally, modified MMP polypeptides, such as tsMMPs, are expressed and purified to be in an inactive form (zymogen form) for subsequent activation as described in the systems and methods provided herein. Hence, following expression, mature forms can be generated by the use of a processing agent and chemical modification, catalysis and/or autocatalysis to remove the prosegment. Generally, a processing agent is chosen that is acceptable for administration to a subject. If necessary, additional purification steps can be performed to remove the processing agent from the purified preparation. In addition, if necessary, additional purification steps can be performed to remove the prosegment from the purified preparation. Activation can be monitored by SDS-PAGE (e.g., a 3 kilodalton shift) and by enzyme activity (cleavage of a fluorogenic substrate). Where an active enzyme is desired, typically, an enzyme is allowed to achieve >75% activation before purification. Typically, MMPs are rendered active by activation cleavage removing the propeptide or prosegment to generate a mature enzyme from a zymogen form. In some applications under nonpermissive temperatures, however, tsMMPs are inactive in their mature form until exposure to the requisite permissive temperature as described herein. For example, many MMPs provided herein are not active or substantially inactive at the non-permissive temperature.

Proteins, such as modified MMP polypeptides, for example, tsMMPs, can be purified using standard protein purification techniques known in the art including but not limited to, SDS-PAGE, size fraction and size exclusion chromatography, ammonium sulfate precipitation and ionic exchange chromatography, such as anion exchange. Affinity purification techniques also can be utilized to improve the efficiency and purity of the preparations. For example, antibodies, receptors and other molecules that bind MMPs can be used in affinity purification. Expression constructs also can be engineered to add an affinity tag to a protein such as a myc epitope, GST fusion or His₆ and affinity purified with myc antibody, glutathione resin and Ni-resin, respectively. Purity can be assessed by any method known in the art including gel electrophoresis and staining and spectrophotometric techniques.

### F. PREPARATION, FORMULATION AND ADMINISTRATION OF tsMMPS

The pharmaceutical compositions provided herein contain modified MMP polypeptides as described herein, for example tsMMPs and/or activity mutants. The compounds can be formulated into suitable pharmaceutical preparations such as solutions, suspensions, tablets, dispersible tablets, pills, capsules, powders, sustained release formulations or elixirs, for oral administrate, as well as transdermal patch preparation and dry powder inhalers. Typically, the compounds are formulated into pharmaceutical compositions using techniques and procedures well known in the art (see e.g., Ansel Introduction to Pharmaceutical Dosage Forms, Fourth Edition, 1985, 126). The pharmaceutical compositions are administered prior to, simultaneously, subsequently or intermittently with an activator that provides the requisite temperature for activation.

A selected modified MMP polypeptide, for example tsMMP, is included in an amount sufficient that, when activated to a mature form and, if necessary, exposed to the permissive temperature, exerts a therapeutically useful effect in the absence of undesirable side effects on the patient treated. The composition containing the modified MMP polypeptide, for example tsMMP, can include a pharmaceutically acceptable carrier. Therapeutically effective concentration can be determined empirically by testing the compounds in known in vitro and in vivo systems, such as the assays provided herein. The concentration of a selected modified MMP polypeptide, for example tsMMP, in the composition depends on absorption, inactivation and excretion rates of the complex, the physicochemical characteristics of the complex, the dosage schedule, and amount administered as well as other factors known to those of skill in the art. For example, it is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope thereof.

The amount of a selected modified MMP polypeptide, for example tsMMP, to be administered for the treatment of a disease or condition, for example an ECM-mediated disease or condition such as cellulite or lymphedema, can be determined by standard clinical techniques. In addition, in vitro assays and animal models can be employed to help identify optimal dosage ranges. The precise dosage, which can be determined empirically, can depend on the particular enzyme, the route of administration, the type of disease to be treated and the seriousness of the disease. Exemplary dosages range from or about 10 μg to 100 mg, particularly 50 μg to 75 mg, 100 μg to 50 mg, 250 μg to 25 mg, 500 μg to 10 mg, 1 mg to 5 mg, or 2 mg to 4 mg. The particular dosage and formulation thereof depends upon the indication and individual. If necessary dosage can be empirically determined. Typically the dosage is administered for indications described herein in a volume of 1-100 ml, particularly, 1-50 ml, 10-50 ml, 10-30 ml, 1-20 ml, or 1-10 ml volumes following reconstitution, such as by addition of an activator (e.g. a cold buffer). Typically, such dosages are from at or about 100 μg to 50 mg, generally 1 mg to 5 mg, in a 10-50 ml final volume.

A modified MMP polypeptide, for example tsMMP, can be administered at once, or can be divided into a number of smaller doses to be administered at intervals of time. Selected modified MMP polypeptides, for example tsMMPs, can be administered in one or more doses over the course of a treatment time for example over several hours, days, weeks, or months. In some cases, continuous administration is useful. It is understood that the precise dosage and course of administration depends on the methods and system of activation contemplated.

Also, it is understood that the precise dosage and duration of treatment is a function of the disease being treated and can be determined empirically using known testing protocols or by extrapolation from in vivo or in vitro test data. It is to be noted that concentrations and dosage values also can vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of compositions and combinations containing them. The compositions can be administered hourly, daily, weekly, monthly, yearly or once. Generally, dosage regimens are chosen to limit toxicity. It should be noted that the attending physician would know how to and when to terminate, interrupt or adjust therapy to lower dosage due to toxicity, or bone marrow, liver or kidney or other tissue dysfunctions. Conversely, the attending physician would also know how to and when to adjust treatment to higher levels if the clinical response is not adequate (precluding toxic side effects).

Pharmaceutically acceptable compositions are prepared in view of approvals for a regulatory agency or other agency prepared in accordance with generally recognized pharmacopeia for use in animals and in humans. Compositions can take the form of solutions, suspensions, emulsion, tablets, pills, capsules, powders, and sustained release formulations. A composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and other such agents. The formulation should suit the mode of administration.

Pharmaceutical compositions can include carriers such as a diluent, adjuvant, excipient, or vehicle with which an enzyme is administered. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents.

Formulations are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable derivatives thereof. Pharmaceutically therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging. Generally, dosage forms or compositions containing active ingredient in the range of 0.005% to 100% with the balance made up from non-toxic carrier can be prepared.

Compositions can be formulated for administration by any route known to those of skill in the art including intramuscular, intravenous, intradermal, intralesional, intraperitoneal injection, subcutaneous, epidural, nasal, oral, vaginal, rectal, topical, local, otic, inhalational, buccal (e.g., sublingual), and transdermal administration or any route. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Compositions also can be administered with other biologically active agents, either sequentially, intermittently or in the same composition. Administration also can include controlled release systems including controlled release formulations and device controlled release, such as by means of a pump.

The most suitable route in any given case depends on a variety of factors, such as the nature of the disease, the progress of the disease, the severity of the disease the particular composition which is used. For purposes herein, it is desired that modified MMP polypeptides, for example tsMMPs, are administered so that they reach the interstitium of skin or tissues. Thus, direct administration under the skin, such as by sub-epidermal administration methods, is contemplated. These include, for example, subcutaneous, intradermal and intramuscular routes of administration. Thus, in one example, local administration can be achieved by injection, such as from a syringe or other article of manufacture containing a injection device such as a needle. Other modes of administration also are contemplated. Pharmaceutical compositions can be formulated in dosage forms appropriate for each route of administration.

In one example, pharmaceutical preparation can be in liquid form, for example, solutions, syrups or suspensions. If provided in liquid form, the pharmaceutical preparation of tsMMP, for example, can be provided as a concentrated preparation to be diluted to a therapeutically effective concentration upon exposure to the permissive temperature, for example, addition of the activator (e.g. a cold buffer). The activator can be added to the preparation prior to administration, or the activator can be added simultaneously, intermittently or sequentially with the tsMMP preparation. Further, if provided in liquid form, the temperature of the preparation can be regulated prior to use in order to achieve a desired temperature for activation. For example, the liquid preparation can be chilled in an ice bucket or in a cold fridge or cold room prior to use and administration. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

In another example, pharmaceutical preparations can be presented in lyophilized form for reconstitution with water or other suitable vehicle before use. For example, the pharmaceutical preparations of tsMMP can be reconstituted with a solution containing an activator at the requisite temperature, generally a cold buffer or liquid solution or a room temperature buffer or liquid solution. Alternatively, once reconstituted, the preparation can be regulated prior to use in order to achieve a desired temperature for activation. For example, the reconstituted liquid preparation can be stored at temperatures that are below the physiological temperature of the body, e.g. at 4° C. to 25° C.

Typically, modified MMP polypeptides provided herein are prepared in compositions containing requisite metals required for activity. For example, MMPs are Zn-dependent and Ca-dependent polypeptides. It is within the level of one of skill in the art to empirically determine the optimal concentration of zinc and calcium required for activity. Where the modified MMP polypeptide is a tsMMP, the optimal concentration of zinc and calcium is a concentration that maintains the temperature-sensitive phenotype. For example, as described herein (e.g. Examples 13 and 14) the presence of zinc can affect the temperature sensitive phenotype of MMP polypeptides. For example, the optimal concentration of ZnCl₂ in MMP compositions provided herein is typically less than 0.01 mM, for example, 0.0005 mM to 0.009 mM, and in particular 0.0005 mM to 0.005 mM, for example 0.001 mM. The optimal concentration of CaCl₂ is typically greater than about 1 mM, for example, 2 mM to 50 mM, in particular 5 mM to 20 mM, for example 10 mM to 15 mM, such as 10 mM. Other metals also can be included in the compositions as required for activity.

Administration methods can be employed to decrease the exposure of modified MMP polypeptides to degradative processes, such as proteolytic degradation and immunological intervention via antigenic and immunogenic responses. Examples of such methods include local administration at the site of treatment. PEGylation of therapeutics has been reported to increase resistance to proteolysis, increase plasma half-life, and decrease antigenicity and immunogenicity. Examples of PEGylation methodologies are known in the art (see for example, Lu and Felix, Int. J. Peptide Protein Res., 43: 127-138, 1994; Lu and Felix, Peptide Res., 6: 142-6, 1993; Felix et al., Int. J. Peptide Res., 46: 253-64, 1995; Benhar et al., J. Biol. Chem., 269: 13398-404, 1994; Brumeanu et al., J Immunol., 154: 3088-95, 1995; see also, Caliceti et al (2003) Adv. Drug Deliv. Rev. 55(10):1261-77 and Molineux (2003) Pharmacotherapy 23 (8 Pt 2):3S-8S). PEGylation also can be used in the delivery of nucleic acid molecules in vivo. For example, PEGylation of adenovirus can increase stability and gene transfer (see, e.g., Cheng et al. (2003) Pharm. Res. 20(9): 1444-51).

1. Injectables, Solutions and Emulsions

Parenteral administration, generally characterized by injection, either subcutaneously, intramuscularly or intradermally is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. The pharmaceutical compositions also may contain other minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the compositions generally includes sub-epidermal routes of administration such as intradermal, subcutaneous and intramuscular administrations. If desired, intravenous administration also is contemplated. Injectables are designed for local and systemic administration. For purposes herein, local administration is desired for direct administration to the affected interstitium. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous. If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEENs 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art. The unit-dose parenteral preparations are packaged in an ampoule, a vial or a syringe with a needle. The volume of liquid solution or reconstituted powder preparation, containing the pharmaceutically active compound, is a function of the disease to be treated and the particular article of manufacture chosen for package. For example, for the treatment of cellulite, it is contemplated that for parenteral injection the injected volume is or is about 10 to 50 milliliters. All preparations for parenteral administration must be sterile, as is known and practiced in the art.

Lyophilized Powders

Of interest herein are lyophilized powders, which can be reconstituted for administration as solutions, emulsions and other mixtures. They may also be reconstituted and formulated as solids or gels.

The sterile, lyophilized powder is prepared by dissolving a compound of inactive enzyme in a buffer solution. The buffer solution may contain an excipient which improves the stability or other pharmacological component of the powder or reconstituted solution, prepared from the powder. Subsequent sterile filtration of the solution followed by lyophilization under standard conditions known to those of skill in the art provides the desired formulation. Briefly, the lyophilized powder is prepared by dissolving an excipient, such as dextrose, sorbital, fructose, corn syrup, xylitol, glycerin, glucose, sucrose or other suitable agent, in a suitable buffer, such as citrate, sodium or potassium phosphate or other such buffer known to those of skill in the art. Then, a selected enzyme is added to the resulting mixture, and stirred until it dissolves. The resulting mixture is sterile filtered or treated to remove particulates and to insure sterility, and apportioned into vials for lyophilization. Each vial will contain a single dosage (1 mg-1 g, generally 1-100 mg, such as 1-5 mg) or multiple dosages of the compound. The lyophilized powder can be stored under appropriate conditions, such as at about 4° C. to room temperature.

Reconstitution of this lyophilized powder with a buffer solution provides a formulation for use in parenteral administration. The solution chosen for reconstitution can be any buffer. For reconstitution about 1 μg-20 mg, preferably 10 μg-1 mg, more preferably about 100 μg is added per mL of buffer or other suitable carrier. The precise amount depends upon the indication treated and selected compound. Such amount can be empirically determined.

2. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e. q., U.S. Pat. Nos. 4,044,126, 4,414,209, and 4,364,923, which describe aerosols for delivery of a steroid useful for treatment inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically diameters of less than 50 microns, preferably less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients also can be administered.

Formulations suitable for transdermal administration are provided. They can be provided in any suitable format, such as discrete patches adapted to remain in intimate contact with the epidermis of the recipient for a prolonged period of time. Such patches contain the active compound in optionally buffered aqueous solution of, for example, 0.1 to 0.2M concentration with respect to the active compound. Formulations suitable for transdermal administration also can be delivered by iontophoresis (see, e.g., Pharmaceutical Research 3(6), 318 (1986)) and typically take the form of an optionally buffered aqueous solution of the active compound.

3. Compositions for Other Routes of Administration

Depending upon the condition treated other routes of administration, such as topical application, transdermal patches, oral and rectal administration are also contemplated herein. For example, pharmaceutical dosage forms for rectal administration are rectal suppositories, capsules and tablets for systemic effect. Rectal suppositories include solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration are manufactured using the same pharmaceutically acceptable substance and by the same methods as for formulations for oral administration.

Formulations suitable for rectal administration can be provided as unit dose suppositories. These can be prepared by admixing the active compound with one or more conventional solid carriers, for example, cocoa butter, and then shaping the resulting mixture.

For oral administration, pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Pharmaceutical compositions also can be administered by controlled release formulations and/or delivery devices (see, e.g., in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566).

Various delivery systems are known and can be used to administer selected tsMMPs, such as but not limited to, encapsulation in liposomes, microparticles, microcapsules, recombinant cells capable of expressing the compound, receptor mediated endocytosis, and delivery of nucleic acid molecules encoding selected matrix-degrading enzymes such as retrovirus delivery systems.

Hence, in certain embodiments, liposomes and/or nanoparticles also can be employed with administration of matrix-degrading enzymes. Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 angstroms containing an aqueous solution in the core.

Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one can operate at the same time. Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use herein, and such particles can be easily made.

4. Activator

Generally, a tsMMP is administered in the presence of an activator that provides the requisite permissive temperature for activation of the enzyme. In other words, tsMMP provided herein are provided for administration at the requisite permissive temperature. Thus, activators provided herein include any that are capable of providing a temperature condition, hot or cold, and that do not exist at the site of administration unless provided exogenously. Thus, tsMMPs can be regulated by controlling the timing and duration of exposure to the temperature condition. An activator is chosen such that it provides a warm or cold temperature depending on the particular enzyme and the permissive temperature requirements provided for activation.

For example where the permissive temperature is 25° C. an activator includes a buffer or other liquid diluent that is at or about 25° C., 24° C., 23° C., 22° C., 21° C., 20° C., 19° C., 18° C., 17° C., 16° C., 15° C., 14° C., 13° C., 12° C., 11° C., 10° C., 9° C., 8° C., 7° C., 6° C., 5° C. or less. In other words, the tsMMP is provided and/or exposed to a buffer or other liquid diluent that is at or about 25° C., 24° C., 23° C., 22° C., 21° C., 20° C., 19° C., 18° C., 17° C., 16° C., 15° C., 14° C., 13° C., 12° C., 11° C., 10° C., 9° C., 8° C., 7° C., 6° C., 5° C. or less. The buffer or liquid can be provided in the same composition as the tsMMP or in a separate composition. When provided separately, it can be administered prior to, simultaneously, subsequently or intermittently from the tsMMP. Upon administration in vivo where the physiologic temperature is at or about 37° C., the temperature of the buffer will warm up to a temperature providing the permissive temperature for activation of the tsMMP (which could occur immediately or almost immediately depending on the temperature of the liquid). Due to the physiologic temperature conditions in vivo, the temperature will warm to non-permissive conditions, thereby resulting in inactivation of the enzyme and temporal control thereof.

In another example, the activator can be a cold pack or a hot pack, depending on the particular enzyme and the permissive temperature provided. Such activators include, but are not limited to ice wraps, gel ice packs, cold therapy, ice packs, cold compress, ice blankets, or other similar items. In other words, the site of locus of administration of the tsMMP can be exposed to the cold pack or hot pack in order to cool or warm the site of administration below or above the physiological temperature of the body, respectively, prior to, concurrently or subsequently with administration of the tsMMP to the same locus. For example, the cold pack can be frozen (e.g. ice pack), or can be a liquid cold pack maintained at a temperature that is 4° C., 5° C., 6° C., 7° C., 8° C., 9° C., 10° C., 11° C., 12° C., 13° C., 14° C., 15° C. or more. A cold or hot pack can be applied directly to the locus of treatment, and generally is applied locally to the skin at the site of administration of the tsMMP. One of skill in the art can empirically determine the length of time required for application depending of the particular target depth of the tissue that is being treated, the particular enzyme that is being used, and other factors based on known testing protocols or extrapolation from in vivo or in vitro test data. The hot pack or cold pack can be applied prior to, subsequently, simultaneously or intermittently from the tsMMP. For example, if the particular enzyme is reversibly active, the cold pack can be applied intermittently over a course of hours or days. It is understood that it is customary for a subject to feel cold, aching and burning and numbness upon administration of a cold pack, and such symptoms can be monitored by the subject or a treating physician.

In particular embodiments, the tsMMP is exposed to a temperature that is at or below the permissive temperature of the body immediately before administration. For example, the tsMMP is stored at a cold temperature and/or is reconstituted in a cold buffer. In some examples, the locus of administration of the tsMMP also is exposed cold by exposure to a cold pack to cool the site of administration below the physiologic temperature of the body. Upon administration of the tsMMP, the tsMMP is exposed to the permissive temperature, which will steadily warm to the nonpermissive physiologic temperature of the body (e.g. about 37° C.). Where the temperature reaches the nonpermissive temperature, the tsMMP is rendered inactive or substantially inactive. Hence, activation of the tsMMP is conditionally controlled. The duration of time of exposure to a permissive temperature below the physiological temperature of the body can be controlled by continued exposure to a cold pack at the site of administration for a predetermined length of time.

In another embodiment, the tsMMP is exposed to a temperature that is at or above the permissive temperature of the body immediately before administration. For example, the tsMMP is stored at a warm temperature and/or is reconstituted in a warm buffer that is above the physiological temperature of the body. In some examples, the locus of administration of the tsMMP also is warmed by exposure to a hot pack to warm the site of administration above the physiologic temperature of the body. Upon administration of the tsMMP, the tsMMP is exposed to the permissive temperature, which will steadily cool to the nonpermissive physiologic temperature of the body (e.g. about 37° C.). Where the temperature reaches the nonpermissive temperature, the tsMMP is rendered inactive or substantially inactive. Hence, activation of the tsMMP is conditionally controlled. The duration of time of exposure to a permissive temperature above the physiological temperature of the body can be controlled by continued exposure to a hot pack at the site of administration for a predetermined length of time.

5. Combination Therapies

Any of the modified MMP polypeptides, for example tsMMPs, described herein can be further co-formulated or co-administered together with, prior to, intermittently with, or subsequent to, other therapeutic or pharmacologic agents or procedures. Such agents include, but are not limited to, other biologics, small molecule compounds, dispersing agents, anesthetics, vasoconstrictors and surgery, and combinations thereof. For example, for any disease or condition, including all those exemplified above, for which other agents and treatments are available, selected modified MMPs, for example tsMMPs, for such diseases and conditions can be used in combination therewith. In another example, a local anesthetic, for example, lidocaine can be administered to provide pain relief. In some examples, the anesthetic can be provided in combination with a vasoconstrictor to increase the duration of the anesthetic effects. Any of the pharmacological agents provided herein can be combined with a dispersion agent that facilitates access into the tissue of pharmacologic agents, for example, following subcutaneous administration. Such substances are known in the art and include, for example, soluble glycosaminoglycanase enzymes such as members of the hyaluronidase glycoprotein family (US20050260186, US20060104968).

Compositions of modified MMPs, for example tsMMPs, provided herein can be co-formulated or co-administered with a local anesthesia. Anesthesias include short-acting and long-lasting local anesthetic drug formulations. Short-acting local anesthetic drug formulations contain lidocaine or a related local anesthetic drug dissolved in saline or other suitable injection vehicle. Typically, local anesthesia with short-acting local anesthetics last approximately 20-30 minutes. Exemplary anesthetics include, for example, non-inhalation local anesthetics such as ambucaines; amoxecaines; amylocalnes; aptocaines; articaines; benoxinates; benzyl alcohols; benzocaines; betoxycaines; biphenamines; bucricaines; bumecaines; bupivacaines; butacaines; butambens; butanilicaines; carbizocaines; chloroprocaine; clibucaines; clodacaines; cocaines; dexivacaines; diamocaines; dibucaines; dyclonines; elucaines; etidocaines; euprocins; fexicaines; fomocaines; heptacaines; hexylcaines; hydroxyprocaines; hydroxytetracaines; isobutambens; ketocaines; leucinocaines; lidocaines; mepivacaines; meprylcaines; octocaines; orthocaines; oxethacaines; oxybuprocaines; phenacaines; pinolcaines; piperocaines; piridocaines; polidocanols; pramocaines; prilocalnes; procaines; propanocaines; propipocaines; propoxycaines; proxymetacaines; pyrrocaines; quatacaines; quinisocaines; risocaines; rodocaines; ropivacaines; salicyl alcohols; suicaines; tetracaines; trapencaines; and trimecaines; as well as various other non-inhalation anesthetics such as alfaxalones; amolanones; etoxadrols; fentanyls; ketamines; levoxadrols; methiturals; methohexitals; midazolams; minaxolones; propanidids; propoxates; pramoxines; propofols; remifentanyls; sufentanyls; tiletamines; and zolamine. The effective amount in the formulation will vary depending on the particular patient, disease to be treated, route of administration and other considerations. Such dosages can be determined empirically.

Due to the short half-life of local anesthetics, it is often desirable to co-administer or co-formulate such anesthetics with a vasoconstrictor. Examples of vasoconstrictors include alpha adrenergic receptor agonists including catecholamines and catecholamine derivatives. Particular examples include, but are not limited to, levonordefrin, epinephrine and norepinephrine. For example, a local anesthetic formulation, such as lidocaine, can be formulated to contain low concentrations of epinephrine or another adrenergic receptor agonist such as levonordefrin. Combining local anesthetics with adrenergic receptor agonists is common in pharmaceutical preparations (see e.g., U.S. Pat. Nos. 7,261,889 and 5,976,556). The vasoconstrictor is necessary to increase the half-life of anesthetics. The vasoconstrictor, such as epinephrine, stimulates alpha-adrenergic receptors on the blood vessels in the injected tissue. This has the effect of constriction the blood vessels in the tissue. The blood vessel constriction causes the local anesthetic to stay in the tissue much longer, resulting in a large increase in the duration of the anesthetic effect.

Generally, a vasoconstrictor is used herein in combination with an anesthetic. The anesthetic agent and vasoconstrictor can be administered together as part of a single pharmaceutical composition or as part of separate pharmaceutical compositions acting together to prolong the effect of the anesthesia, so long as the vasoconstrictor acts to constrict the blood vessels in the vicinity of the administered anesthetic agent. In one example, the anesthetic agent and vasoconstrictor are administered together in solution. In addition, the anesthetic agent and vasoconstrictor can be formulated together or separate from the activatable matrix-degrading enzyme and activator. Single formulations are preferred. The anesthetic agent and vasoconstrictor can be administered by injection, by infiltration or by topical administration, e.g., as part of a gel or paste. Typically, the anesthetic agent and vasoconstrictor are administered by injection directly into the site to be anesthetized, for example, by subcutaneous administration. The effective amount in the formulation will vary depending on the particular patient, disease to be treated, route of administration and other considerations. Such dosages can be determined empirically. For example, exemplary amounts of lidocaine are or are about 10 mg to 1000 mg, 100 mg to 500 mg, 200 mg to 400 mg, 20 mg to 60 mg, or 30 mg to 50 mg. The dosage of lidocaine administered will vary depending on the individual and the route of administration. Epinephrine can be administered in amounts such as, for example, 10 μg to 5 mg, 50 μg to 1 mg, 50 μg to 500 μg, 50 μg to 250 μg, 100 μg to 500 μg, 200 μg to 400 μg, 1 mg to 5 mg or 2 mg to 4 mg. Typically, epinephrine can be combined with lidocaine in a 1:100,000 to 1:200,000 dilution, which means that 100 ml of anesthetic contains 0.5 to 1 mg of epinephrine. Volumes administered can be adjusted depending on the disease to be treated and the route of administration. It is contemplated herein that 1-100 ml, 1-50 ml, 10-50 ml, 10-30 ml, 1-20 ml, or 1-10 ml, typically 10-50 ml of an anesthetic/vasoconstrictor formulation can be administered subcutanously for the treatment of an ECM-mediated disease or condition, such as cellulite. The administration can be subsequent, simultaneous or intermittent with administration of an activatable matrix-degrading enzyme and activator.

Compositions of modified MMP polypeptides, for example tsMMPs, provided herein also can be co-formulated or co-administered with a dispersion agent. The dispersion agent also can be co-formulated or co-administered with other pharmacological agents, such as anesthetics, vasoconstrictors, or other biologic agents. Exemplary of dispersion agents are glycosaminoglycanases that open channels in the interstitial space through degradation of glycosaminoglycans. These channels can remain relatively open for a period of 24-48 hours depending on dose and formulation. Such channels can be used to facilitate the diffusion of exogenously added molecules such as fluids, small molecules, proteins (such as matrix degrading enzymes), nucleic acids and gene therapy vectors and other molecules less than about 500 nm in size. In addition, it is thought that the formation of such channels can facilitate bulk fluid flow within an interstitial space, which can in turn promote the dispersion or movement of a solute (such as a detectable molecule or other diagnostic agent, an anesthetic or other tissue-modifying agent, a pharmacologic or pharmaceutically effective agent, or a cosmetic or other esthetic agent) that is effectively carried by the fluid in a process sometimes referred to as “convective transport” or simply convection. Such convective transport can substantially exceed the rate and cumulative effects of molecular diffusion and can thus cause the therapeutic or other administered molecule to more rapidly and effectively perfuse a tissue. Furthermore, when an agent, such as a modified MMP, for example a tsMMP, anesthetic or other agent, is co-formulated or co-administered with a glycosaminoglycanase and both are injected into a relatively confined local site, such as a site of non-intravenous parenteral administration (e.g., intradermal, subcutaneous, intramuscular, or into or around other internal tissues, organs or other relatively confined spaces within the body), then the fluid associated with the administered dose can both provide a local driving force (i.e. hydrostatic pressure) as well as lower impedance to flow (by opening channels within the interstitial matrix), both of which could increase fluid flow, and with it convective transport of the therapeutic agent or other molecule contained within the fluid. As a result, the use of glycosaminoglycanases can have substantial utility for improving the bioavailability as well as manipulating other pharmacokinetic and/or pharmacodynamic characteristics of co-formulated or co-administered agents, such as matrix degrading enzymes.

Hyaluronidases

Exemplary of glycosaminoglycanases are hyaluronidases. Hyaluronidases are a family of enzymes that degrade hyaluronic acid. By catalyzing the hydrolysis of hyaluronic acid, a major constituent of the interstitial barrier, hyaluronidase lowers the viscosity of hyaluronic acid, thereby increasing tissue permeability. There are three general classes of hyaluronidases: Mammalian-type hyaluronidases, (EC 3.2.1.35) which are endo-beta-N-acetylhexosaminidases with tetrasaccharides and hexasaccharides as the major end products. They have both hydrolytic and transglycosidase activities, and can degrade hyaluronan and chondroitin sulfates (CS), generally C4-S and C6-S; Bacterial hyaluronidases (EC 4.2.99.1), which degrade hyaluronan and to various extents, CS and DS. They are endo-beta-N-acetylhexosaminidases that operate by a beta elimination reaction that yields primarily disaccharide end products; and Hyaluronidases (EC 3.2.1.36) from leeches, other parasites, and crustaceans that are endo-beta-glucuronidases that generate tetrasaccharide and hexasaccharide end products through hydrolysis of the beta 1-3 linkage.

There are six hyaluronidase-like genes in the human genome, HYAL1 (SEQ ID NO:3469), HYAL2 (SEQ ID NO: 3470), HYAL3 (SEQ ID NO:3471), HYAL4 (SEQ ID NO:3472), PH20/SPAM1 (SEQ ID NO:3473) and one expressed pseudogene, HYALPI. Among hyaluronidases, PH20 is the prototypical neutral active enzyme, while the others exhibit no catalytic activity towards hyaluronan or any known substrates, or are active only under acidic pH conditions. The hyaluronidase-like enzymes can also be characterized by those which are generally locked to the plasma membrane via a glycosylphosphatidyl inositol anchor such as human HYAL2 and human PH20 (Danilkovitch-Miagkova, et al. (2003) Proc Natl Acad Sci USA. 100(8):4580-5), and those which are generally soluble such as human HYAL1 (Frost et al., (1997) Biochem Biophys Res Commun. 236(1): 10-5). N-linked glycosylation of some hyaluronidases can be very important for their catalytic activity and stability. While altering the type of glycan modifying a glycoprotein can have dramatic affects on a protein's antigenicity, structural folding, solubility, and stability, many enzymes are not thought to require glycosylation for optimal enzyme activity. Hyaluronidases are, therefore, unique in this regard, in that removal of N-linked glycosylation can result in near complete inactivation of the hyaluronidase activity. For such hyaluronidases, the presence of N-linked glycans is critical for generating an active enzyme.

Human PH20 (also known as sperm surface protein PH20) is naturally involved in sperm-egg adhesion and aids penetration by sperm of the layer of cumulus cells by digesting hyaluronic acid. The PH20 mRNA transcript (corresponding to nucleotides 1058-2503 of the sequence set forth in SEQ ID NO:3474) is normally translated to generate a 509 amino acid precursor protein containing a 35 amino acid signal sequence at the N-terminus (amino acid residue positions 1-35) and a 19 amino acid GPI anchor at the C-terminus (corresponding to amino acid residues 491-509). The precursor sequence is set forth in SEQ ID NO:3473. An mRNA transcript containing a mutation of C to T at nucleotide position 2188 of the sequence of nucleic acids set forth in SEQ ID NO:3474 also exists and is a silent mutation resulting in the translated product set forth in SEQ ID NO: 3473. The mature PH20 is, therefore, a 474 amino acid polypeptide corresponding to amino acids 36-509 of the sequence of amino acids set forth in SEQ ID NO:3473. There are potential N-linked glycosylation sites required for hyaluronidases activity at N82, N166, N235, N254, N368, N393, N490 of human PH20 exemplified in SEQ ID NO: 3473. Disulfide bonds form between the cysteine residues C60 and C351 and between C224 and C238 (corresponding to amino acids set forth in SEQ ID NO:3473) to form the core hyaluronidase domain. Additional cysteines are required in the carboxy terminus for neutral enzyme catalytic activity such that amino acids 36 to 464 of SEQ ID NO:3473 contain the minimally active human PH20 hyaluronidase domain.

Soluble forms of recombinant human PH20 have been produced and can be used in the methods described herein for co-administration or co-formulation with tsMMPs, activators, anesthetics, vasoconstrictors, other pharmacologic or therapeutic agents, or combinations thereof, to permit the diffusion into tissues. The production of such soluble forms of PH20 is described in related application Ser. Nos. 11/065,716 and 11/238,171. Soluble forms include, but are not limited to, any having C-terminal truncations to generate polypeptides containing amino acid 1 to amino acid 442, 443, 444, 445, 446 and 447 of the sequence of amino acids set forth in SEQ ID NOS:3476-3481. Exemplary of such a polypeptides are those generated from a nucleic acid molecule encoding amino acids 1-482 set forth in SEQ ID NO:3475. Resulting purified rHuPH20 can be heterogenous due to peptidases present in the culture medium upon production and purification. Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g. DG44 CHO cells).

The soluble PH20 can be administered by any suitable route as described elsewhere herein. Typically, administration is by parenteral administration, such as by intradermal, intramuscular, subcutaneous or intravascular administration. The compounds provided herein can be formulated for parenteral administration by injection, e.g., by bolus injection or continuous infusion. Formulations for injection can be presented in unit dosage form, e.g., in ampoules or in multi-dose containers, with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use. For example, provided herein are parenteral formulations containing an effective amount of soluble PH20, such as 10 Units to 500,000 Units, 100 Units to 100,000 Units, 500 Units to 50,000 Units, 1000 Units to 10,000 Units, 5000 Units to 7500 Units, 5000 Units to 50,000 Units, or 1,000 Units to 10,000 Units, generally 10,000 to 50,000 Units, in a stabilized solution or suspension or a lyophilized from. The formulations can be provided in unit-dose forms such as, but not limited to, ampoules, syringes and individually packaged tablets or capsules. The dispersing agent can be administered alone, or with other pharmacologically effective agents in a total volume of 1-100 ml, 1-50 ml, 10-50 ml, 10-30 ml, 1-20 ml, or 1-10 ml, typically 10-50 ml.

In one example of a combination therapy, it is contemplated herein that an anesthetic, vasoconstrictor and dispersion agent are co-administered or co-formulated with a tsMMP to be administered subsequently, simultaneously or intermittently therewith. An exemplary formulation is one containing lidocaine, epinephrine and a soluble PH20, for example, a soluble PH20 set forth in SEQ ID NO:3476. Soluble PH20 can be mixed directly with lidocaine (Xylocalne), and optionally with epinephrine. The formulation can be prepared in a unit dosage form, such as in a syringe. For example, the lidocaine/epinephrine/soluble PH20 formulation can be provided in a volume, such as 1-100 ml, 1-50 ml, 10-50 ml, 10-30 ml, 1-20 ml, or 1-10 ml, typically 10-50 ml, prepackaged in a syringe for use.

In the combination therapies, the other pharmacologic agents, such as a lidocaine/epinephrine/soluble PH20 formulation, can be co-administered together with or in close temporal proximity to the administration of an activatable matrix-degrading enzyme (and activator). Typically it is preferred that an anesthetic and/or dispersion agent be administered shortly before (e.g. 5 to 60 minutes before) or, for maximal convenience, together with the pharmacologic agent. As will be appreciated by those of skill in the art, the desired proximity of co-administration depends in significant part on the effective half lives of the agents in the particular tissue setting, and the particular disease being treated, and can be readily optimized by testing the effects of administering the agents at varying times in suitable models, such as in suitable animal models.

### G. PACKAGING AND ARTICLES OF MANUFACTURE OF tsMMPS

Pharmaceutical compounds of modified MMPs, for example tsMMPs, or nucleic acids encoding modified MMPs, or a derivative or variant thereof can be packaged as articles of manufacture containing packaging material, a pharmaceutical composition which is effective for treating the disease or disorder, and a label that indicates that selected modified MMP or nucleic acid molecule is to be used for treating the disease or disorder. Instructions for use can be provided. For example, instructions can be provided that specify that the tsMMP is to be reconstituted with the accompanying liquid buffer or solution, kept cold, immediately before administration. Instructions also can be provided for administration of a cold pack at the site of administration of the tsMMP. Combinations of a modified MMP, for example tsMMP, or derivative or variant thereof and an activator (e.g. cold pack or liquid buffer) also can be packaged in an article of manufacture. In some examples, combinations also can include a processing agent.

The articles of manufacture provided herein contain packaging materials. Packaging materials for use in packaging pharmaceutical products are well known to those of skill in the art. See, for example, U.S. Pat. Nos. 5,323,907, 5,052,558 and 5,033,252, each of which is incorporated herein in its entirety. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. The articles of manufacture can include a needle or other injection device so as to facilitate administration (e.g. sub-epidermal administration) for local injection purposes. A wide array of formulations of the compounds and compositions provided herein are contemplated as are a variety of treatments for any ECM-mediated disease or disorder.

The choice of package depends on the tsMMP and activator (if included therewith), and whether such compositions will be packaged together or separately. In general, the packaging is non-reactive with the compositions contained therein such that activation of the tsMMP does not occur prior to addition of the activator. In one example, the modified MMP can be packaged in lyophilized form with a buffer or diluent for reconstitution. The buffer or diluent can be stored separately at a temperature providing the activated condition, or can be provided in a form capable of providing the activating condition when desired. For example, instructions can be provided to chill or cool and or warm the buffer or diluent before use. Alternatively, instructions can be provided to activate the enzyme by use of a cold pack or heat pack at the locus of administration, for example, following reconstitution of the enzyme and administration thereof.

Exposure to the activator can occur at any time preceding administration of the tsMMP by exposure of the tsMMP to the requisite permissive temperature. For example, the container can have a single compartment containing the tsMMP and being amenable to addition of the activator (e.g. cold or room temperature liquid buffer or solution) by the user, for example through an opening in the compartment. Any container or other article of manufacture that is amenable to having a defining space for containment of the tsMMP and that is amenable to simple manipulation to permit addition of the final components necessary for activation is contemplated. The activator is added prior to use. Exposure to the activator also can occur following administration to the interstitium. For example, if heat is the activator, a tsMMP can be administered and the local injection site subjected to heat. If colder temperatures are the activator, a tsMMP can be administered and the local injection site subjected to cold, e.g. by a cold pack.

In other examples, the tsMMP is packaged in a container with the activator such that activation of the matrix-degrading enzyme is amenable to activation by the user at will in the container. Generally, examples of such containers include those that have an enclosed, defined space that contains the matrix-degrading enzyme, and a separate enclosed, defined space containing the activator such that the two spaces are separated by a readily removable membrane which, upon removal, permits the components to mix and thereby react, resulting in activation of the protease. The container can be stored under conditions such that the activator is at or near the requisite permissive temperature for activation of the MMP. Alternatively, only the side of the container containing the activator can be cooled or warmed to the desired temperature (e.g. by exposing it to an ice wrap or other temperature condition) just prior to use and reconstitution of the enzyme. Any container or other article of manufacture is contemplated, so long as the tsMMP is separated from the activator. Exposure of the activator to the tsMMP is prior to use. For example, the physical separation means are those that are readily removed by the user, to permit mixing, resulting in activation of the enzyme. For example, an article of manufacture can contain a tsMMP in one compartment and an activator (e.g. cold or room temperature liquid buffer or solution) in an adjacent compartment. The compartments are separated by a dividing member, such as a membrane, that, upon compression of the article or manufacture ruptures permitting separated components to mix. For suitable embodiments see e.g., containers described in U.S. Pat. Nos. 3,539,794 and 5,171,081.

Following are some examples of the packaging requirements of various end uses of activatable matrix-degrading enzymes. These are offered as examples only and in no way are intended as limiting.

1. Single Chamber Apparatus

Among the simplest embodiments herein, are those in which the apparatus contains a single chamber or container and, if needed, ejection means. Single chamber housings or containers include any item in which a tsMMP is included in the container. The tsMMP is housed in the vessel in liquid phase or as a powder or other paste or other convenient composition. The vessel or liquid can be stored at a temperature that is at or below the permissive temperature and/or cooled to at or below the permissive temperature prior to administration. Alternatively, a tsMMP is reconstituted with an appropriate liquid diluent or buffer and the activator is applied locally to the site of administration (e.g. cold pack) or is administered separately at the site of administration. Kits containing the item and the activator also are provided.

2. Dual Chamber Apparatus

An example of an apparatus contemplated for use herein is a dual chamber container. In general, this apparatus has two chambers or compartments thereby maintaining the tsMMP from an activator capable of providing the activating condition until activation is desired. The apparatus can include a mixing chamber to permit mixing of the components prior to dispensing from the apparatus. Alternatively, mixing can occur by ejection of the activator from one chamber into a second chamber containing the tsMMP. For example, the activatable tsMMP can be provided in lyophilized form, and reconstitution can be achieved by ejection of the activator (e.g. e.g. cold or room temperature buffer or liquid solution) from a first chamber into the second chamber containing the lyophilized enzyme. It is understood that the temperature of the entire apparatus can be controlled together and/or the chamber containing the activator can be brought to the desired temperature prior to use and reconstitution of the enzyme.

In one embodiment, a dual chamber apparatus employs a mechanical pump mechanism in its operation. In such an example, the dispensing apparatus maintains the components in separate chambers. A pump mechanism is operated to withdraw the contents from each chamber and into a mixing chamber, or from one chamber into the second chamber. Upon mixing, the mixed composition is activated by reaction of the components in the chambers. The pump mechanism can be manually operated, for example, by a plunger. Exemplary of such dual chamber apparatus include dual chamber syringes (see e.g., U.S. Pat. Nos. 6,972,005, 6,692,468, 5,971,953, 4,529,403, 4,202,314, 4,214,584, 4,983,164, 5,788,670, 5,395,326; and Intl. Patent Appl. Nos. WO2007006030, WO2001047584).

Another embodiment of a dual chamber fluid dispensing apparatus contemplated for use herein takes the form of a compressible bottle or tube or other similar device. The device has two compartments within it that keep the components separated. The cap of the device can serve as a mixing chamber, a mixing chamber can be positioned between the two chambers and the cap, or mixing can be achieved within one of the chambers. The components are forced by compression from the separate compartments into the mixing chamber. They are then dispensed from the mixing chamber. For example, the mixed contents can be removed from the device by attaching a plunger/syringe apparatus to the dispensing end and withdrawing the contents therethrough. Such devices are known in the art (see e.g., Intl. Patent Appln. No. WO1994015848).

3. Kits

Selected modified MMP polypeptides, for example tsMMPs, and/or articles of manufacture thereof also can be provided as kits. The kits optionally can include an activator and/or processing agent. Kits can include a pharmaceutical composition described herein and an item for administration provided as an article of manufacture. For example a selected tsMMP can be supplied with a device for administration, such as a syringe, an inhaler, a dosage cup, a dropper, or an applicator. The compositions can be contained in the item for administration or can be provided separately to be added later. Generally, kits contain an item with a tsMMP, and optionally a processing agent and/or an activator capable of providing the activating condition. The kit can, optionally, include instructions for application including dosages, dosing regimens, instructions for using the activator (e.g. warming or cooling the buffer or applying a cold or hot pack), and instructions for modes of administration. Kits also can include a pharmaceutical composition described herein and an item for diagnosis. For example, such kits can include an item for measuring the concentration, amount or activity of the selected protease in a subject.

### H. METHODS OF ASSESSING ACTIVITY OF tsMMPS

1. Methods of Assessing Enzymatic Activity

Modified MMPs, including tsMMPs, can be tested for their enzymatic activity against known substrates. Activity assessment can be performed in the presence or absence of an activator and at varying temperatures. Activity assessments can be performed on conditioned medium or other supernatants or on purified protein.

Enzymatic activity can be assessed by assaying for substrate cleavage using known substrates of the enzyme. The substrates can be in the form of a purified protein or provided as peptide substrates. For example, enzymatic activity of MMP can be assessed by cleavage of collagen. Cleavage of a purified protein by an enzyme can be assessed using any method of protein detection, including, but not limited to, HPLC, SDS-PAGE analysis, ELISA, Western blotting, immunohistochemistry, immunoprecipitation, NH2-terminal sequencing, protein labeling and fluorometric methods. For example, Example 5 describes an assay to assess enzymatic activity for cleavage of a collagen that is FITC-labeled. Fluorescence of the supernatant is an indication of the enzymatic activity of the protein and can be normalized to protein concentration and a standard curve for specific activity assessment.

In addition, enzymatic activity can be assessed on tetrapeptide substrates. The use of fluorogenic groups on the substrates facilitates detection of cleavage. For example, substrates can be provided as fluorogenically tagged tetrapeptides of the peptide substrate, such as an ACC- or 7-amino-4-methyl courmarin (AMC)-tetrapeptide. Other fluorogenic groups are known and can be used and coupled to protein or peptide substrates. These include, for example, 7-amino-4-methyl-2-quinolinone (AMeq), 2-naphthylamine (HNap) and 7 amino-4-methylcoumarin (NHMec) (Sarath et al. “Protease Assay Methods,” in Proteolytic Enzymes: A Practical Approach. Ed. Robert J. Beynon and Judith S. Bond. Oxford University Press, 2001. pp. 45-76). Peptide substrates are known to one of skill in the art, as are exemplary fluorogenic peptide substrates. For example, exemplary substrates for MMP include, peptide IX, designated as Mca-K-P-L-G-L-Dpa-A-R-NH₂ (SEQ ID NO:707; Mca=(7-Methoxycoumarin-4-yl)acetyl; Dpa=N-3-(2,4,-Dinitrophenyl)-L-2,3-diaminopropionyl; R&D Systems, Minneapolis, Minn., Cat# ESOIO) and variations thereof such as with different fluorogenic groups. Enzyme assays to measure enzymatic activity by fluorescence intensity are standard and are typically performed as a function of incubation time of the enzyme and substrate (see e.g., Dehrmann et al. (1995) Arch. Biochem. Biophys., 324:93-98; Barrett et al. (1981) Methods Enzymol., 80:536-561). Exemplary assays using fluorescence substrates are described in Example 2 herein.

While detection of fluorogenic compounds can be accomplished using a fluorometer, detection can be accomplished by a variety of other methods well known to those of skill in the art. Thus, for example, when the fluorophores emit in the visible wavelengths, detection can be simply by visual inspection of fluorescence in response to excitation by a light source. Detection also can be by means of an image analysis system utilizing a video camera interfaced to a digitizer or other image acquisition system. Detection also can be by visualization through a filter, as under a fluorescence microscope. The microscope can provide a signal that is simply visualized by the operator. Alternatively, the signal can be recorded on photographic film or using a video analysis system. The signal also can simply be quantified in real time using either an image analysis system or a photometer.

Thus, for example, a basic assay for enzyme activity of a sample involves suspending or dissolving the sample in a buffer (at the pH optima of the particular protease being assayed) adding to the buffer a fluorogenic enzyme peptide indicator, and monitoring the resulting change in fluorescence using a spectrofluorometer as shown in e.g., Harris et al., (1998) J Biol Chem 273:27364. The spectrofluorometer is set to excite the fluorophore at the excitation wavelength of the fluorophore. The fluorogenic enzyme indicator is a substrate sequence of an enzyme (e.g. of a protease) that changes in fluorescence due to a protease cleaving the indicator.

2. Methods of Assessing ECM Degradation

The degradation of extracellular matrix proteins by modified MMPs, for example tsMMPs, including, but not limited to, those described above, such as tsMMP-1, can be assessed in vitro or in vivo. Assays for such assessment are known to those of skill in the art, and can be used to test the activities of a variety of modified MMPs, for example tsMMPs, on a variety of extracellular matrix proteins, including, but not limited to collagen (I, II, III and IV), fibronectin, vitronectin and proteoglycans. Assays can be performed at permissive and non-permissive temperatures. Experiments also can be performed in the presence of an MMP that is not modified to be temperature sensitive. It is understood that assays for enzymatic activity are performed subsequent to activation of the enzyme by a processing agent. As a further control, activity of the zymogen enzyme also can be assessed.

a. In Vitro Assays

Exemplary in vitro assays include assays to assess the degradation products of extracellular matrix proteins following incubation with a modified MMP, for example tsMMP. In some examples, the assays detect a single, specific degradation product. In other examples, the assays detect multiple degradation products, the identity of which may or may not be known. Assessment of degradation products can be performed using methods well known in the art including, but not limited to, HPLC, CE, Mass spectrometry, SDS-PAGE analysis, ELISA, Western blotting, immunohistochemistry, immunoprecipitation, NH2-terminal sequencing, and protein labeling. Extracellular matrix degradation products can be visualized, for example, by SDS-PAGE analysis following incubation with MMPs, such as tsMMPs, for an appropriate amount of time at an appropriate temperature. For example, collagen can be incubated with mature modified MMP, for example tsMMP, and subjected to SDS-PAGE using, for example, a 4-20% Tris/glycine gel to separate the products. Coomassie staining of the gel facilitates visualization of smaller degradation products, or disappearance of collagen bands, compared to intact collagen. Immunoblotting using, for example, a polyclonal Ig specific to the extracellular matrix protein also can be used to visualize the degradation products following separation with SDS-PAGE.

Assays that specifically detect a single product following degradation of an extracellular matrix protein also are known in the art and can be used to assess the ability of a tsMMP to degrade an extracellular matrix protein. For example, the hydroxyproline (HP) assay can be used to measure degradation of collagen. 4-hydroxyproline is a modified imino acid that makes up approximately 12% of the weight of collagen. HP assays measure the amount of solubilized collagen by determining the amount of HP in the supernatant following incubation with a matrix-degrading enzyme (see e.g., Reddy and Enwemeka (1996) Clinical Biochemistry 29:225-229). Measurement of HP can be effected by, for example, colorimetric methods, high performance liquid chromatography, mass spectrometry and enzymatic methods (see e.g., Edwards et al., (1980) Clin. Chim. Acta 104:161-167; Green (1992) Anal. Biochem. 201:265-269; Tredget et al., (1990) Anal. Biochem. 190:259-265; Ito et al., (1985) Anal. Biochem. 151:510-514; Garnero et al. (1998) J. Biol. Chem. 273:32347-32352).

The collagen source used in such in vitro assays can include, but is not limited to, commercially available purified collagen, bone particles, skin, cartilage and rat tail tendon. Collagenolytic activity of a modified MMP, such as tsMMP such as tsMMP-1, can be assessed by incubating the activated enzyme with an insoluble collagen suspension, followed by hydrolysis, such as with HCl. The amount of hydroxyproline derived from the solubilized (degraded) collagen can be determined by spectrophotometric methods, such as measuring the absorbance at 550 nm following incubation with Ehrlich's reagent. In some examples, the collagen source is rat or pig skin explant that is surgically removed from anesthetized animals and then perfused with the tsMMP, for example, tsMMP-1, prior to, subsequently, simultaneously or intermittently with a temperature activator. HP levels in the perfusates can then be assessed. In a modification of this method, the effect on the fibrous septae in the explants can also be assessed. Briefly, following perfusion with the enzyme, the explants are cut into small pieces and embedded in paraffin and analyzed by microscopy following Masson's Trichrome staining for visualization of collagen. The number of collagen fibrous septae can be visualized and compared to tissue that has not been treated with a enzyme.

Assays to detect degradation of specific collagens also are known in the art. Such assays can employ immunological methods to detect a degradation product unique to the specific collagen. For example, the degradation of collagen I by some MMPs releases telopeptides with different epitopes that can be detected using immunoassays. Such assays detect the cross-linked N-telopeptides (Tx) and the cross-linked C-telopeptides (CTx and ICTP), each of which contain unique epitopes. Typically, CTx assays utilize the CrossLaps (Nordic Biosciences) antibodies that recognize the 8 amino acid sequence EKAHD-β-GGR octapeptide, where the aspartic acid is in β-isomerized configuration, in the C-terminal telopeptide region of the α1 chain (Eastell (2001) Bone Markers: Biochemical and Clinical Perspectives, pg 40). Immunoassays to detect ICTP also are known in the art and can be used to detect degradation of collagen I (U.S. Pat. No. 5,538,853). In other examples, immunoassays, such as, for example, ELISAs, can be used to detect NTx following incubation of collagen type I with proteases such as an MMP (Atley et al., (2000) Bone, 26:241-247). Other antibodies and assays specific for degraded collagens are known in the art and can be used to detect degradation by matrix-degrading enzymes. These include antibodies and assays specific for degraded collagen I (Hartmann et al (1990) Clin. Chem. 36:421-426), collagen II (Hollander et al (1994) J. Clin. Invest. 93:1722-1732), collagen III (U.S. Pat. No. 5,342,756), and collagen IV (Wilkinson et al (1990) Anal. Biochem. 185:294-6).

b. In Vivo Assays

Assays to detect the in vivo degradation of ECM also are known in the art. Such assays can utilize the methods described above to detect, for example, hydroxyproline and N- and C-telopeptides and degraded collagens or other ECM in biological samples such as urine, blood, serum and tissue. Detection of degraded ECM can be performed following administration to the patient of one or more enzymes. Detection of pyridinoline (PYD) and deoxypyridinoline (DPYD), also can be used to assess degradation of collagen. Also known as hydroxylysylpyridinoline and lysylpyridinoline, respectively, PYD and DPYD are the two nonreducible trivalent cross-links that stabilize type I collagen chains and are released during the degradation of mature collagen fibrils. Pyridinoline is abundant in bone and cartilage, whereas deoxypyridinoline is largely confined to bone. Type III collagen also contains pyridinoline cross-links at the amino terminus. Total PYD and DPYD can be measured, for example, in hydrolyzed urine samples or serum by fluorometric detection after reversed-phase HPLC (Hata et al (1995) Clin. Chimica. Acta. 235:221-227).

c. Non-Human Animal Models

Non-human animal models can be used to assess the activity of matrix-degrading enzymes. For example, non-human animals can be used as models for a disease or condition. Non-human animals can be injected with disease and/or phenotype-inducing substances prior to administration of enzymes. Genetic models also are useful. Animals, such as mice, can be generated which mimic a disease or condition by the overexpression, underexpression or knock-out of one or more genes. For example, animal models are known in the art for conditions including, but not limited to, Peyronie's Disease (Davila et al. (2004) Biol. Reprod., 71:1568-1577), tendinosis (Warden et al., (2006) Br. J. Sports Med. 41:232-240) and scleroderma (Yamamoto (2005) Cur. Rheum. Rev. 1:105-109).

Non-human animals also can be used to test the activity of enzymes in vivo in a non-diseased animal. For example, enzymes can be administered to, non-human animals, such as, a mouse, rat or pig, and the level of ECM degradation can be determined. In some examples, the animals are used to obtain explants for ex vivo assessment of ECM degradation. In other examples, ECM degradation is assessed in vivo. For example, collagen degradation of the skin of anesthetized animals can be assessed. Briefly, an MMP, such as a tsMMP-1, is perfused prior to, simultaneously, subsequently or intermittently with a temperature activator via insertion of a needle into the dermal layer of the skin of the tail. Perfusate fractions are collected from the tail skin and analyzed for collagen degradation by hydroxyproline analysis. Other methods can be used to detect degradation including, but not limited to, any of the assays described above, such as immunoassays to detect specific degradation products.

### I. EXEMPLARY METHODS OF TREATING DISEASES OR DEFECTS OF ECM

The modified MMPs, for example tsMMPs, provided herein can be used for treatment of any condition mediated by any one or more ECM components. This section provides exemplary uses of, and administration methods for, modified MMPs, such as tsMMPs. These described therapies are exemplary and do not limit the applications of enzymes. Such methods include, but are not limited to, methods of treatment of any ECMI condition or disease that is caused by excess, aberrant or accumulated expression of any one or more ECM component. Exemplary of diseases or conditions to be treated are any mediated by collagen, elastin, fibronectin, or a glycosaminoglycan such as a proteoglycan. For example, exemplary of collagen-mediated diseases or disorders include, but are not limited to, cellulite, Dupuytren's disease (also called Dupuytren's contracture), Peyronie's disease, frozen shoulder, chronic tendinosis or scar tissue of the tendons, localized scleroderma and lymphedema. It is within the skill of a treating physician to identify such diseases or conditions.

The particular disease or condition to be treated dictates the enzyme that is selected. For example, treatment of a collagen-mediated disease or disorder can be effected by administration of a modified MMP, for example tsMMP, that cleaves collagen. For example, a modified MMP-1, for example tsMMP-1, can be selected for cleaving collagen. Such MMPs include modified forms on any MMP listed above in Table 5, and/or known to one of skill in the art. tsMMPs, and systems and methods for activation can be chosen accordingly to treat a particular disease or condition.

Treatment of diseases and conditions with modified MMPs, for example tsMMPs, can be effected by any suitable route of administration using suitable formulations as described herein including, but not limited to, subcutaneous injection, intramuscular, intradermal, oral, and topical and transdermal administration. As described above, a route of administration of modified MMPs, for example tsMMPs, typically is chosen that results in administration under the skin directly to the affected site. Exemplary of such routes of administration include, but are not limited to, subcutaneous, intramuscular, or intradermal.

If necessary, a particular dosage and duration and treatment protocol can be empirically determined or extrapolated. For example, exemplary doses of recombinant and native active MMPs or modified MMPs, for example tsMMPs, can be used as a starting point to determine appropriate dosages. Dosage levels can be determined based on a variety of factors, such as body weight of the individual, general health, age, the activity of the specific compound employed, sex, diet, time of administration, rate of excretion, drug combination, the severity and course of the disease, and the patient's disposition to the disease and the judgment of the treating physician. The amount of active ingredient that can be combined with the carrier materials to produce a single dosage form will vary depending upon the particular matrix-degrading enzyme, the host treated, the particular mode of administration, and the activating condition required for activation, and/or the predetermined or length of time in which activation is desired. The pharmaceutical compositions typically should provide a dosage of from about 1 μg/ml to about 20 mg/ml. Generally, dosages are from or about 10 μg/ml to 1 mg/ml, typically about 100 μg/ml, per single dosage administration. It is understood that the amount to administer will be a function of the tsMMP and the activating condition chosen, the indication treated, and possibly side effects that will be tolerated. Dosages can be empirically determined using recognized models for each disorder. Also, as described elsewhere herein, modified MMPs, for example tsMMPs, can be administered in combination with other agents sequentially, simultaneously or intermittently. Exemplary of such agents include, but are not limited to, lidocaine, epinephrine, a dispersing agent such as hyaluronidase and combinations thereof.

Upon improvement of a patient's condition, a maintenance dose of a compound or compositions can be administered, if necessary; and the dosage, the dosage form, or frequency of administration, or a combination thereof can be modified. In some cases, a subject can require intermittent treatment on a long-term basis upon any recurrence of disease symptoms.

Descriptions of the involvement of collagen to collagen-mediated diseases or conditions is provided below as an example of the role of ECM components in diverse disease and conditions. Such descriptions are meant to be exemplary only and are not limited to a particular modified MMP or tsMMP or to a particular ECM-mediated diseases or conditions. One of skill in the art can select a modified MMP, for example, tsMMP and activating condition for activation thereof, to be used in the treatment of any desired ECM-mediated disease, based on the ability of a particular enzyme to cleave or degrade an ECM component involved in the particular disease or condition. For example, as described herein, MMP-1 cleaves type I and type III collagens, such as those abundant in the skin. Hence, a modified MMP-1 can be used for treatments, uses and processes for treating a collagen-mediated disease or condition. The particular treatment and dosage can be determined by one of skill in the art. Considerations in assessing treatment include, for example, the disease to be treated, the ECM component involved in the disease, the severity and course of the disease, whether the modified MMP, for example tsMMP, is administered for preventive or therapeutic purposes, previous therapy, the patient's clinical history and response to therapy, and the discretion of the attending physician.

Collagen-Mediated Diseases or Conditions

Collagen is a major structural constituent of mammalian organisms and makes up a large portion of the total protein content of the skin and other parts of the animal body. Numerous diseases and conditions are associated with excess collagen deposition, for example, due to erratic accumulation of fibrous tissue rich in collagen or other causes. Collagen-mediated diseases or conditions (also referred to as fibrotic tissue disorders) are known to one of skill in the art (see e.g., published U.S. Application No. 20070224183; U.S. Pat. Nos. 6,353,028; 6,060,474; 6,566,331; 6,294,350). Excess collagen has been associated with diseases and conditions, such as, but not limited to, fibrotic diseases or conditions resulting in scar formation, cellulite, Dupuytren's syndrome, Peyronie's disease, frozen shoulder, localized scleroderma, lymphedema, Interstitial cystitis (IC), Telangrectase, Barrett's metaplasia, Pneumatosis cytoides intestinalis, collagenous colitis. For example, disfiguring conditions of the skin, such as wrinkling, cellulite formation and neoplastic fibrosis result from excessive collagen deposition, which produces unwanted binding and distortion of normal tissue architecture.

Modified MMP polypeptides, for example tsMMPs described herein, including but not limited to modified MMP-1 and tsMMP-1, can be used to treat collagen-mediated diseases or conditions. Exemplary of tsMMPs for treatment of diseases and conditions described herein is a tsMMP-1 that is more active at a non-permissive temperature that is below the physiological temperature of the body such as at or about 25° C. compared to the nonpermissive physiologic temperature at the site of administration. For example, temporary cooling of the extracellular matrix, such as the skin interstitium, can be achieved by infusing a cold buffered solution or other liquid directly at the affected site and/or applying a cold pack directly to the locus of administration. In one example, a cold buffer can be administered via sub-epidermal administration, i.e. under the skin, such that administration is effected directly at the site where ECM components are present and accumulated. Other methods of activation can be employed, and are known to one of skill in the art in view of the descriptions herein.

a. Cellulite

Modified MMP polypeptides, for example, tsMMPs, such as those described herein, including a modified MMP-1 polypeptide or tsMMP-1, can be used to treat cellulite. In normal adipose tissues, a fine mesh of blood vessels and Iymph vessels supplies the tissue with necessary nutrients and oxygen, and takes care of the removal of metabolized products. For example, triglycerides are stored in individual adipocytes that are grouped into capillary rich lobules. Each fat lobule is composed of adipocytes. Vertical strands of collagen fibers named fibrous septae separate the fat lobules and tether the overlying superficial fascia to the underlying muscle.

Cellulite is typically characterized by dermal deterioration due to a breakdown in blood vessel integrity and a loss of capillary networks in the dermal and subdermal levels of the skin. The vascular deterioration tends to decrease the dermal metabolism. This decreased metabolism hinders protein synthesis and repair processes, which results in dermal thinning. The condition is further characterized by fat cells becoming engorged with lipids, swelling and clumping together, as well as excess fluid retention in the dermal and subdermal regions of the skin. The accumulation of fat globules or adipose cells creates a need for a bigger blood supply to provide extra nourishment. To provide the blood to tissues, new capillaries are formed, which release more filtrate resulting in a saturation of tissues with interstitial fluid causing edema in the adipose tissues. Abundant reticular fibers in the interstitial tissues accumulate and thicken around the aggregated adipose cells; they form capsules or septa, which gradually transform into collagen fibers and are felt as nodules. The formation of these septa further occludes fat cells. Collagen fibers are also laid down in the interstitial tissue spaces, rendering the connective tissue sclerotic (hard).

Hence, as the condition further progresses, hard nodules of fat cells and clumps of fats surrounded by septa form in the dermal region. This leads to the surface of the skin displaying considerable heterogeneity and being characterized as having a “cottage cheese” or “orange peel” appearance. The dimpling occurs when the fibrous septae that connect the skin to the dermis and deeper tissue layers tighten and pull in the skin. Thus, the “orange peel” appearance of cellulite is due to the deformation of the fat lobules as a result of outward forces on the adipose tissue. The fat lobules can be large, for example up to 1 cm wide, and easily protrude into the overlying dermis, causing a visible deformation on the surface of the skin. The net result is the undulating appearance of the outer skin as the fat pushes upwards. As the connective septae run in the same direction as these outward forces, they can offer no counter force to keep the adipose from protruding into the dermis.

Cellulite is more prevalent among females than males. The prevalence of cellulite is estimated between 60% and 80% of the female population and its severity tends to worsen with obesity. Recently, a published study showed by in vivo magnetic resonance imaging that women with cellulite have a higher percentage of perpendicular fibrous septae than women without cellulite or men (Querleux et al., (2002) Skin Research and Technology, 8:118-124). Cellulite occurs most often on the hips, thighs and upper arms. For example, premenopausal females tend to accumulate fat subcutaneously, primarily in the gluteal/thigh areas where cellulite is most common. Clinically, cellulite is accompanied by symptoms that include thinning of the epidermis, reduction and breakdown of the microvasculature leading to subdermal accumulations of fluids, and subdermal agglomerations of fatty tissues.

b. Dupuytren's Disease

Modified MMP polypeptides, for example tsMMPs, such as a modified MMP-1 or a tsMMP-1 such as those described herein, can be used to treat Dupuytren's syndrome (also called Dupuytren's contracture). Dupuytren's contracture (also known as Morbus Dupuytren) is a fixed flexion contracture of the hand where the fingers bend towards the palm and cannot be fully extended. A similar lesion sometimes occurs in the foot. The connective tissue within the hand becomes abnormally thick and is accompanied by the presence of nodules containing fibroblasts and collagen, particularly type III collagen. The fibrous cord of collagen is often interspersed with a septa-like arrangement of adipose tissue. These present clinically as mattress-type “lumps” of varying sized and in Dupuytren's disease are termed nodules. This can cause the fingers to curl, and can result in impaired function of the fingers, especially the small and ring fingers. Dupuytren's disease occurs predominantly in men. It is generally found in middle aged and elderly persons, those of Northern European ancestry, and in those with certain chronic illnesses such as diabetes, alcoholism and smoking.

Dupuytren's disease is a slowly progressive disease that occurs over many years causing fixed flexion deformities in the metacarpophalangeal (MP) and proximal interphalangeal (PIP) joints of the fingers. The small and ring fingers are the most often affected. The disease progresses through three stages (Luck et al. (1959) J Bone Joint Surg., 41A:635-664). The initial proliferative stage is characterized by nodule formation in the palmar fascia in which a cell known as the myofibroblast appears and begins to proliferate. The involutional or mid-disease stage involves myofibroblast proliferation and active type III collagen formation. In the last or residual phase, the nodule disappears leaving acellular tissue and thick bands of collagen. The ratio of type III collagen to type I collagen increases. Treatment of Dupuytren's disease with an activatable-matrix degrading enzyme is typically in the mid-disease and residual disease stages.

c. Peyronie's Disease

Modified MMP-1, for example tsMMPs, such as a modified MMP-1 or a tsMMP-1 such as those described herein, can be used to treat Peyronie's disease. Peyronie's disease is a connective tissue disorder involving the growth of fibrous plaques in the soft tissue of the penis affecting as many as 1-4% of men. Collagen is the major component of the plaque in Peyronie's disease. Specifically, the fibrosing process occurs in the tunica albuginea, a fibrous envelope surrounding the penile corpora cavemosa. The pain and disfigurement associated with Peyronie's disease relate to the physical structure of the penis in which is found two erectile rods, called the corpora cavernosa, a conduit (the urethra) through which urine flows from the bladder, and the tunica which separates the cavernosa from the outer layers of skin of the penis. A person exhibiting Peyronie's disease will have formation(s) of plaque or scar tissue between the tunica and these outer layers of the skin (referred to as “subdermal” in this application). The scarring or plaque accumulation of the tunica reduces its elasticity causes such that, in the affected area, it will not stretch to the same degree (if at all) as the surrounding, unaffected tissues. Thus, the erect penis bends in the direction of the scar or plaque accumulation, often with associated pain of some degree. In all but minor manifestations of Peyronie's disease, the patient has some degree of sexual dysfunction. In more severe cases, sexual intercourse is either impossible, or is so painful as to be effectively prohibitive.

Empirical evidence indicates an incidence of Peyronie's disease in approximately one percent of the male population. Although the disease occurs mostly in middle-aged men, younger and older men can acquire it. About 30 percent of men with Peyronie's disease also develop fibrosis (hardened cells) in other elastic tissues of the body, such as on the hand or foot. Common examples of such other conditions include Dupuytren's contracture of the hand and Ledderhose Fibrosis of the foot.

d. Ledderhose Fibrosis

Modified MMP polypeptides, for example tsMMPs, for example, a modified MMP-1 or tsMMP-1 such as those described herein, can be used to treat Ledderhose fibrosis. Ledderhose fibrosis is similar to Dupuytren's disease and Peyronie's disease, except that the fibrosis due to fibroblast proliferation and collagen deposition occurs in the foot. Ledderhose disease is characterized by plantar fibrosis over the medial sole of the foot, and is sometimes referred to as plantar fibrosis.

e. Stiff Joints

Modified MMP polypeptides, for example tsMMPs, such as a modified MMP-1 or a tsMMP-1 such as those described herein, can be used to treat stiffjoints, for example, frozen shoulder. Frozen shoulder (adhesive capsulitis) is a chronic fibrozing condition of the capsule of the joint characterized by pain and loss of motion or stiffness in the shoulder. It affects about 2% of the general population. Frozen shoulder results from increased fibroblast matrix synthesis. The synthesis is caused by an excessive inflammatory response resulting in the overproduction of cytokines and growth factors. Fibroblasts and myofibroblasts lay down a dense matrix of collagen in particular, type-I and type-III collagen within the capsule of the shoulder. This results in a scarred contracted shoulder capsule and causes joint stiffness.

Other examples of stiffjoints include, but are not limited to, those caused by capsular contractures, adhesive capsulitis and arthrofibrosis, which result from musculoskeletal surgery. Such stiffjoints can occur in joints, including, for example, joints of the knees, shoulders, elbows, ankles and hips. Like frozen shoulder, such joint diseases are caused by increased matrix synthesis and scar formation. The stiff joints inevitably can cause abnormally high forces to be transmitted to the articular cartilage of the affected area. Over time, these forces result in the development of degenerative joint disease and arthritis. For example, in arthrofibrosis and capsular contracture, fibroblasts form excessive amounts of matrix in response to local trauma, such as joint dislocation.

f. Existing Scars

Modified MMP-1, for example tsMMPs, such as a modified MMP-1 or tsMMP-1 such as those described herein, can be used to treat existing scars. Collagen is particularly important in the wound healing process and in the process of natural aging, where it is produced by fibroblast cells. In some cases, however, an exaggerated healing response can result in the production of copious amounts of healing tissue (ground substance), also termed scar tissue. For example, various skin traumas such as burns, surgery, infection, wounds and accident are often characterized by the erratic accumulation of fibrous tissue rich in collagen. There also is often an increased proteoglycan content. In addition to the replacement of the normal tissue that has been damaged or destroyed, excessive and disfiguring deposits of new tissue sometimes form during the healing process. The excess collagen deposition has been attributed to a disturbance in the balance between collagen synthesis and collagen degradation. Including among scars are, for example, chronic tendinosis or scar tissue of the tendons, surgical adhesions, keloids, hypertrophic scars, and depressed scars.

i. Surgical Adhesions

Surgical adhesions are attachments of organs or tissues to each other through scar formation, which can cause severe clinical problems. The formation of some scar tissue after surgery or tissue injury is normal. In some cases, however, the scar tissue overgrows the region of injury and creates surgical adhesions, which tend to restrict the normal mobility and function of affected body parts. In particular, fibroblast proliferation and matrix synthesis is increased locally following such soft tissue injury. Adhesions then form when the body attempts to repair tissue by inducing a healing response. For example, this healing process can occur between two or more otherwise healthy separate structures (such as between loops of bowel following abdominal surgery). Alternately, following local trauma to a peripheral nerve, fibrous adhesions can form, resulting in severe pain during normal movement.

ii. Keloids

Keloids are scars of connective tissue containing hyperplastic masses that occur in the dermis and adjacent subcutaneous tissue, most commonly following trauma. Keloids generally are fibrous nodules that can vary in color from pink or red to dark brown. Keloids form in scar tissue as a result of overgrowth of collagen, which participates in wound repair. Keloid lesions are formed when local skin fibroblasts undergo vigorous hyperplasia and proliferation in response to local stimuli. The resulting lesion can result in a lump many times larger than the original scar. In addition to occur as a result of wound or other trauma, keloids also can form from piercing, pimples, a scratch, severe acne, chickenpox scarring, infection at a wound site, repeated trauma to an area, or excessive skin tension during wound closure.

iii. Hypertrophic Scars

Hypertrophic scars are raised scars that form at the site of wounds. They generally do not grow beyond the boundaries of the original wound. Like keloid scars, hypertrophic scars are a result of the body overproducing collagen.

iv. Depressed Scars

Depressed scars generally result from an inflammatory episode and are characterized by contractions of the skin, and leave a cosmetically displeasing and permanent scar. The most common example is scarring that occurs following inflammatory acne. The depression occurs as a normal consequence of wound healing, and the scar tissue causing the depression is predominantly made up of collagen resulting from fibroblast proliferation and metabolism.

g. Scleroderma

Modified MMP polypeptides, for example tsMMPs, for example, a modified MMP-1 or a tsMMP-1 such as those described herein, can be used to treat scleroderma. Scleroderma is characterized by a thickening of the collagen. The more common form of the disease, localized scleroderma, affects only the skin, usually in just a few places, and sometimes the face. It is sometimes referred to as CREST syndrome. Symptoms include hardening of the skin and associated scarring. The skin also appears reddish or scaly, and blood vessels can be more visible. In more serious cases, scleroderma can affect the blood vessels and internal organs. Diffuse scleroderma can be fatal as a result of heart, kidney lung or intestinal damage, due to musculoskeletal, pulmonary, gastrointestinal, renal and other complications.

The condition is characterized by collagen buildup leading to loss of elasticity. The overproduction of collagen has been attributed to autoimmune dysfunction, resulting in accumulation of T cells and production of cytokines and other proteins that stimulate collagen deposition from fibroblasts.

h. Lymphedema

Modified MMP polypeptides, for example tsMMPs, for example, a modified MMP-1 or tsMMP-1 such as those described herein, can be used to treat lymphedema. Lymphedema is an accumulation of lymphatic fluid that causes swelling in the arms and legs. Lymphedema can progress to include skin changes such as, for example, lymphostatic fibrosis, sclerosis and papillomas (benign skin tumors) and swelling. Tissue changes associated with lymphedema include proliferation of connective tissue cells, such as fibroblasts, production of collagen fibers, an increase in fatty deposits and fibrotic changes. These changes occur first at the lower extremities, i.e. the fingers and toes. Lymphedema can be identified based on the degree of enlargement of the extremities. For example, one method to assess lymphedema is based on identification of 2-cm or 3-cm difference between four comparative points of the involved and uninvolved extremities.

i. Collagenous Colitis

Modified MMP polypeptides, for example tsMMPs, such as a modified MMP-1 or a tsMMP-1 such as those described herein, can be used to treat collagenous colitis. Collagenous colitis was first described as chronic watery diarrhea (Lindstrom et al. (1976) Pathol. Eur., 11:87-89). Collagenous colitis is characterized by collagen deposition, likely resulting from an imbalance between collagen production by mucosal fibroblasts and collagen degradation. It results in secretory diarrhea. The incidence of collagenous colitis is similar to primary biliary cirrhosis. The disease has an annual incidence of 1.8 per 100,000 and a prevalence of 15.7 per 100,000, which is similar to primary biliary cirrhosis (12.8 per 100,000) and lower than ulcerative colitis (234 per 100,000), Crohn's disease (146 per 100,000) or celiac disease (5 per 100,000). In patients with chronic diarrhea, about 0.3 to 5% have collagenous colitis. Collagenous colitis is an inflammatory disease resulting in increased production of cytokines and other agents that stimulate the proliferation of fibroblasts, resulting in increased collagen accumulation.

2. Spinal Pathologies

As described herein, the modified MMPs provided herein can be used to treat diseases and conditions of the ECM or involving the ECM. These include spinal pathologies, typically referred to as herniated disc or bulging discs, that can be treated by administering an MMP provided herein and activating as described herein. Herniated discs that can be treated include protruded and extruded discs. A protruded disc is one that is intact but bulging. In an extruded disk, the fibrous wrapper has torn and nucleus pulposus (NP) has oozed out, but is still connected to the disk. While the NP is not the cause of the herniation, the NP contributes to pressure on the nerves causing pain. The NP contains hyaluronic acid, chondrocytes, collagen fibrils, and proteoglycan aggrecans that have hyaluronic long chains which attract water. Attached to each hyaluronic chain are side chains of chondroitin sulfate and keratan sulfate.

Herniated discs have been treated with chemonucleolytic drugs, such as chymopapain and a collagenase, typically by local introduction of the drug into the disc. A chemonucleolytic drug degrades one or more components of the NP, thereby relieving pressure. Chemonucleolysis is effective on protruded and extruded disks. Chemonucleolysis has been used treat lumbar (lower) spine and cervical (upper spine) hernias. Hence, the MMPs provided herein can be used as chemonucleolytic drugs and administered, such as by injection, to the affected disc, under conditions that activate the MMP.

### J. EXAMPLES

The following examples are included for illustrative purposes only and are not intended to limit the scope of the invention.

### Example 1

### Cloning and Expression of hMMP-1

A. Cloning and High-Throughput Expression of hMMP-1 Library

In this example, a human matrix metalloprotease 1 (hMMP-1) library was created by cloning DNA encoding human MMP-1 into a plasmid followed by transformation and protein expression/isolation. The library was created by introducing mutations in a parent human MMP-1 DNA sequence having the sequence of nucleotides set forth in SEQ ID NO:706, which encodes the inactive zymogen proMMP-1 (set forth in SEQ ID NO:2), to generate single amino acid variants of MMP-1 across the catalytic domain and proline rich linker domain of the polypeptide. The hMMP-1 library was designed to contain at least 15 amino acid variants at each of 178 amino acids positions within the catalytic domain (amino acids 81-242 of SEQ ID NO:2) and the linker region (amino acids 243-258 of SEQ ID NO:2) of human MMP-1 (See Table 7, below).

TABLE 7
hMMP-1 Library

| Amino | | |
|---|---|---|
| Acid | Amino Acid Substitutions | SEQ ID NOS |
| F81 | E; H; R; C; Q; T; S; G; M; W; | 780-781, 783-784, 786, 787, |
| | I; V; L; A; P | 789-797 |
| V82 | R; C; N; Q; T; Y; S; G; F; M; | 802-816 |
| | W; I; L; A; P | |
| L83 | D; E; H; R; C; Q; T; Y; S; G; | 817-819, 821-822, 824-825, |
| | M; W; I; A; P | 826, 827-828, 830-832, 834- |
| | | 835 |
| T84 | D; E; H; R; C; Q; Y; S; G; F; | 836-838, 840-841, 843-847, |
| | I; V; L; A; P | 850-854 |
| E85 | K; R; C; N; Q; T; Y; S; G; F; | 857-867, 870-873 |
| | M; V; L; A; P | |
| G86 | D; H; K; C; N; T; Y; S; F; | 874, 876-877, 879-880, 882- |
| | M; W; I; V; L; P | 890, 892 |
| N87 | E; H; R; C; Q; Y; S; G; F; M; | 894-895, 897-899, 901-905, |
| | I; V; L; A;P | 907-911 |
| P88 | D; E; H; K; R; C; Q; T; Y; G; | 912-917,919-921,923,926- |
| | W; I; V; L; A | 930 |
| R89 | E; H; K; N; T; Y; S; G; F; M; | 932-934, 936, 938-944, 946- |
| | W; V; L; A; P | 949 |
| W90 | E; H; R; N; Q; T; S; G; F; M; | 951-952, 954, 956-958, 960- |
| | I; V; L; A; P | 968 |
| E91 | D; H; R; C; N; T; Y; S; G; F; | 969-970, 972-974, 976-980, |
| | W; I; V; L; A | 982-986 |
| Q92 | E; K; R; N; T; Y; S; G; F; W; | 989, 991-992, 994-999, 1001- |
| | I; V; L; A; P | 1006 |
| T93 | D; E; K; R; N; S; G; F; M; W; | 1007-1008, 1010-1011, 1013, |
| | I; V; L; A; P | 1016-1025 |
| H94 | D; E; R; N; T; S; G; F; M; W; | 1026-1027, 1029, 1031, 1033, |
| | I; V; L; A; P | 1035-1044 |
| L95 | D; E; H; K; R; C; T; Y; S; G; | 3-8, 11-14, 17-21 |
| | W; I; V; A; P | |
| T96 | E; H; R; C; N; Q; S; G; F; W; | 1046-1047, 1049-1052, 1054- |
| | I; V; L; A; P | 1056, 1058-1063 |
| Y97 | D; E; H; K; R; N; Q; T; S; G; | 1064-1068, 1070-1074, 1077, |
| | W; V; L; A; P | 1079-1082 |
| R98 | D; E; H; K; C; Y; S; G; F; M; | 1083-1087, 1091-1096, 1098- |
| | W; V; L; A; P | 1101 |
| I99 | E; H; R; C; N; Q; T; Y; S; G; | 1103-1104, 1106-1114, 1116- |
| | F; W; V; L; A; P | 1120 |
| E100 | D; H; R; N; T; Y; S; G; F; M; | 497-498, 500, 502, 504-513, |
| | W; I; V; L; P | 515 |
| N101 | D; H; K; R; C; T; Y; S; F; M; | 1121, 1123-1126, 1128-1130, |
| | W; V; L A; P | 1132-1134, 1136-1139 |
| Y102 | D; E; K; R; C; N; Q; S; G; F; M; | 1140-1141, 1143-1147, 1149- |
| | V; L; A; P | 1152, 1155-1158 |
| T103 | D; E; K; R; C; N; Q; Y; S; G; | 516-517, 519-526, 529,531- |
| | W; V; L; A; P | 534 |
| P104 | D; E; H; R; C; Q; T; Y; S; G; | 1159-1161, 1163-1164, 1166- |
| | F; M; V; L; A | 1172, 1175-1177 |
| D105 | E; R; C; N; T; S; G; F; M; W; | 22, 25-27, 29, 31-40 |
| | I; V; L; A; P | |
| L106 | D; H; R; C; N; T; Y; S; G; F; | 1178, 1180, 1182-1184, 1186- |
| | M; I; V; A; P | 1191, 1193-1196 |
| P107 | D; K; R; C; T; Y; S; G; F; M; | 1197, 1200-1202, 1205-1215 |
| | W; I; V; L; A | |
| R108 | E; K; C; N; T; Y; S; G; F; W | 1217, 1219-1221, 1223-1227, |
| | I; V; L; A; P | 1229-1234 |
| A109 | D; E; H; R; N; Q; T; Y; S; G; | 1235-1237, 1239, 1241-1246, |
| | M; W; I V; L; | 1248-1252 |
| D110 | H; R; C; Q; T; Y; S; G; F; M; | 1255, 1257-1258, 1260-1266, |
| | I; V; L; A; P | 1268-1272 |
| V111 | D; E; K; R; C; Q; T; Y; S; G; | 1273-1274, 1276-1278, 1280- |
| | W; I; L; A; P | 1284, 1287-1291 |
| D112 | H; K; R; C; Q; T; Y; S; G; F; | 1293-1296, 1298-1310 |
| | M; W; I; V; L; A; P | |
| H113 | D; E; R; N; T; Y; S; G; F; M; | 1311-1312, 1314, 1316, 1318- |
| | W; V;L; A;P | 1324, 1326-1329 |
| A114 | E; R; C; N; Q; T; S; G; F; M; | 1331, 1334-1338, 1340-1348 |
| | W; I; V; L; P | |
| I115 | D; E; H; K; R; C; Q; T; S; G; | 1349-1354, 1356-1357, 1359- |
| | F; W; V; L; P | 1361, 1363-1365, 1367 |
| E116 | D; H; K; R; C; N; Q; S; G; F; | 1368-1374, 1377-1380, 1382, |
| | M; I; L; A; P | 1384-1386 |
| K117 | D; E; H; R; N; Q; T; Y; S; G; | 1387-1390, 1392-1398, 1400, |
| | F; W; L; A; P | 1403-1405 |
| A118 | D; E; H; K; R; Q; T; S; G; F; | 1406-1410, 1413-1414, 1416- |
| | W; I; V; L; P | 1418, 1420-1424 |
| F119 | E; H; K; R; C; N; T; Y; S; G; | 1426-1431, 1433-1436, 1438, |
| | W; V; L; A; P | 1440-1443 |
| Q120 | D; E; H; K; R; C; N; T; Y; G; | 1444-1452, 1454, 1456-1457, |
| | M; W; V; A; P | 1459, 1461-1462 |
| L121 | E; H; K; R; C; N; Q; T; S; G; | 1464-1471, 1473-1475, 1478- |
| | F; I; V; A; P | 1481 |
| W122 | E; H; K; R; N; Q; T; Y; S; G; | 1483-1486, 1488-1494, 1497- |
| | F; V; L; A; P | 1500 |
| S123 | D; H; K; R; C; N; Q; T; Y; G; | 1501, 1503-1519 |
| | F; M; W; I; V; L; A; P | |
| N124 | D; K; R; C; T; S; G; F; M; W; | 1520, 1523-1525, 1527, 1529- |
| | I; V; L; A; P | 1538 |
| V125 | D; E; H; R; C; Q; T; Y; S; G; | 1539-1541, 1543-1544, 1546- |
| | F; M; W A; P | 1553, 1556-1557 |
| T126 | E; H; K; R; N; Q; S; G; F; M; | 1559-1562, 1564-1565, 1567- |
| | W; V; L; A; P | 1571, 1573-1576 |
| P127 | E; H; K; R; C; Q; T; S; F; M; | 1578-1582, 1584-1585, 1587, |
| | W; I; V; L; A | 1589-1595 |
| L128 | D; K; R; C; Q; T; S; G; F; M; | 1596, 1599-1601, 1603-1604, |
| | W; I; V; A; P | 1606-1614 |
| T129 | E; H; K; R; C; Y; S; G; F; M; | 1616-1620, 1623-1627, 1629- |
| | I; V; L; A; P | 1633 |
| F130 | E; H; K; R; C; N; T; Y; S; G; | 1635-1640, 1642-1645, 1648- |
| | I; V; L; A; P | 1652 |
| T131 | D; E; H; R; C; Q; Y; S; G; F; | 1653-1655, 1657-1658, 1660- |
| | M; I; L; A; P | 1665, 1667, 1669-1671 |
| K132 | D; E; H; R; T; Y; S; G; F; M; | 1672-1675, 1679-1684, 1686- |
| | I; V; L; A; P | 1690 |
| V133 | D; E; H; K; R; C; N; T; S; G; | 1691-1697, 1699, 1701-1702, |
| | M; W; L; A; P | 1704-1705, 1707-1709 |
| S134 | D; E; H; K; R; C; N; Q; T; Y; | |
| | G; V; A; P | 1710-1720, 1725-1728 |
| E135 | D; H; R; N; Q; T; S; F; M; W; | 1729-1730, 1732, 1734-1736, |
| | I; V; L; A; P | 1738, 1740-1747 |
| G136 | D; E; H; R; C; N; T; S; M; W; | 1748-1750, 1752-1754, 1756, |
| | I; V; L; A; P | 1758, 1760-1766 |
| Q137 | E; H; K; R; C; N; T; Y; S; G; | 1768-1778, 1780, 1783-1785 |
| | F; W; L; A; P | |
| A138 | D; E; H; R; C; Q; T; S; G; M; | 1786-1788, 1790-1791, 1793- |
| | W; I;V ; L; P | 1794, 1796-1797, 1799-1804 |
| D139 | E; H; R; C; N; Y; S; G; F; M; | 1805-1806, 1808-1810, 1813- |
| | W; I; V; L; A; P | 1823 |
| I140 | D; E; H; K; R; C; T; Y; G; F; | 1824-1829, 1832-1833, 1835- |
| | M; W; V; L; A | 1841 |
| M141 | D; E; H; R; C; N; T; Y; S; G; | 1843-1845, 1847-1849, 1851- |
| | W; I; L; A; P | 1854, 1856-1857, 1859-1861 |
| I142 | K; R; N; Q; T; Y; S; G; F; M; | 1865-1866, 1868-1880 |
| | W; V; L; A; P | |
| S143 | E; H; R; C; N; Q; T; Y; G; M; | 1882-1883, 1885-1891, 1893- |
| | W; I; L; A; P | 1895, 1897-1899 |
| F144 | E; H; K; R; C; N; Q; T; S; G; | 1901-1908, 1910-1913, 1915- |
| | M; W; V; L;P | 1916, 1918 |
| V145 | D; E; H; K; R; C; N; Q; T; S; | 1919-1927, 1929-1930, 1933, |
| | G; W; L; A;P | 1935-1937 |
| R146 | D; E; H; K; C; N; Q; T; Y; S; | 1938-1947, 1949, 1953-1956 |
| | F; V; L; A; P | |
| G147 | E; H; R; C; Q; T; S; F; M; W; | 1958-1959, 1961-1962, 1964- |
| | I; V; L; A; P | 1965, 1967-1975 |
| D148 | E; K; R; C; N; T; S; G; M; W; | 1976, 1978-1981, 1983, 1985- |
| | I; V; L; A; P | 1986, 1988-1994 |
| H149 | E; R; C; N; Q; T; Y; S; G; W; | 1996, 1998-2005, 2008-2013 |
| | I; V; L; A; P | |
| R150 | D; E; H; K; N; T; S; G; M; W; | 41-44, 46, 48, 50-51, 53-59 |
| | I; V; L; A; P | |
| D151 | K; R; N; Q; T; Y; S; C; F; M; | 62-63, 65-73, 75-78 |
| | W; V; L; A; P | |
| N152 | D; H; K; R; C; T; Y; S; G; F; | 2014, 2016-2019, 2021-2025, |
| | W; I; L; A; P | 2027-2028, 2030-2032 |
| S153 | D; H; K; R; C; Q; T; Y; G; F; | 535, 537-540, 542-546, 549- |
| | I; V; L; A; P | 553 |
| P154 | H; K; R; C; N; Q; T; Y; S; F; | 2035-2043, 2045, 2047-2051 |
| | W; I; V; L; A | |
| F155 | E; H; R; N; Q; T; Y; S; G; M; | 80-81, 83, 85-92, 94-97 |
| | W; V; L; A; P | |
| D156 | E; H; K; R; C; T; Y; S; G; M; | 98-102, 105-108, 110-111, |
| | W; V; L; A; P | 113-116 |
| G157 | D; H; K; R; N; Q; T; Y; S; F; | 2052, 2054-2056, 2059-2065, |
| | M; V; L; A; P | 2068-2071 |
| P158 | D; K; R; C; N; Q; T; Y; S; G; | 2072, 2075-2084, 2086-2090 |
| | F; W; I; V; L; A; | |
| G159 | E; K; R; C; Q; T; Y; S; M; W; | 118, 120-122, 124-127, 129- |
| | I; V; L; A; P | 135 |
| G160 | E; H; R; C; N; Q; T; S; M; W; | 2092-2093, 2095-2099, 2101, |
| | I; V; L; A; P | 2103-2109 |
| N161 | E; H; R; C; Q; T; Y; S; G; F; | 2111-2112, 2114-2121, 2123- |
| | W; I; V; L; P | 2126, 2128 |
| L162 | D; E; R; C; Q; T; Y; S; G; F; | 2129-2130, 2133-2134, 2136- |
| | M; W; I; A; P | 2144, 2146-2147 |
| A163 | E; K; R; C; N; Q; T; Y; S; G; | 2149, 2151-2160, 2163-2166 |
| | F; I; V; L; P | |
| H164 | E; K; R; C; N; Q; Y; S; G; F; | 2168-2173, 2175-2179, 2182- |
| | M; V; L; A; P | 2185 |
| A165 | D; H; K; R; N; Q; T; S; G; F; | 2186, 2188-2190, 2192-2194, |
| | M; W; V; L; P | 2196-2200, 2202-2204 |
| F166 | E; H; K; R; C; N; S; G; M; | 2206-2211, 2215-2223 |
| | W; I; V; L; A; P | |
| Q167 | D; E; K; R; N; T; Y; S; G; F; | 2224-2225, 2227-2228, 2230- |
| | M; V; L; A; P | 2236, 2239-2242 |
| P168 | D; H; R; C; N; T; S; G; F; M; | 2243, 2245, 2247-2249, 2251, |
| | W; I; V; L; A | 2253-2261 |
| G169 | D; E; H; R; C; Q; T; S; M; W; | 2262-2264, 2266-2267, 2269- |
| | I; V; L; A; P | 2270, 2272, 2274-2280 |
| P170 | D; H; K; R; C; Q; T; S; G; F; | 2281, 2283-2286, 2288-2289, |
| | M; W; I; L; A | 2291-2296, 2298-2299 |
| G171 | D; E; H; K; R; C; N; Q; Y; S; | 554-561, 563-564, 566-567, |
| | M; W; L; A; P | 570-572 |
| I172 | D; E; R; C; N; Q; T; Y; G; M; | 2300-2301, 2304-2309, 2311, |
| | W; V; L; A; P | 2313-2318 |
| G173 | D; K; R; C; N; T; Y; S; F; M; | 2319, 2322-2325, 2327-2332, |
| | W; V; L; A; P | 2334-2337 |
| G174 | D; E; H; R; N; T; Y; S; F; M; | 2338-2340, 2342, 2344, 2346- |
| | W; V; L; A; P | 2351, 2353-2356 |
| D175 | E; H; R; C; N; Q; T; Y; S; G; | 2357-2358, 2360-2368, 2371- |
| | F; I; V; L; A; P | 2375 |
| A176 | D; E; K; R; C; N; Q; T; S; G; | 136-137, 139-144, 146-148, |
| | F; W; V; L; P | 150, 152-154 |
| H177 | D; R; C; N; Q; T; Y; S; G; W; | 2376, 2379-2386, 2389-2394 |
| | I; V; L; A; P | |
| F178 | E; H; K; R; C; Q; T; Y; S; G; | 2396-2400, 2402-2406, 2408- |
| | W; I; V; L; A; P | 2413 |
| D179 | E; K; R; C; N; Q; T; S; G; W; | 155, 157-162, 164-165, 168- |
| | I; V; L; A; P | 173 |
| E180 | D; K; R; C; N; Q; T; Y; S; G; | 174, 176-186, 188, 191-192 |
| | F; M; I; A; P | |
| D181 | E; K; R; C; Q; T; Y; S; G; F; | 193, 195-197, 199-205, 208- |
| | M; V; L; A; P | 211 |
| E182 | D; R; C; Q; T; Y; S; G; F; M; | 212, 215-216, 218-226, 228- |
| | W; I; L; A; P | 230 |
| R183 | E; H; K; C; N; T; S; G; M; W; | 2415-2419, 2421, 2423-2424, |
| | I; V; L; A; P | 2426-2432 |
| W184 | E; H; R; N; Q; T; S; G; F; M; | 2434-2435, 2437, 2439-2441, |
| | I; V; L; A; P | 2443-2451 |
| T185 | D; E; H; R; C; N; Q; Y; S; G; | 231-233, 235-241, 244, 246- |
| | W; V; L; A; P | 249 |
| N186 | D; E; H; R; C; Q; T; Y; S; G; | 2452-2454, 2456-2463, 2467- |
| | F; V; L; A; P | 2470 |
| N187 | D; H; K; R; C; T; S; C; F; M; | 250, 252-255, 257, 259-264, |
| | W; I; L; A; P | 266-268 |
| F188 | D; E; H; K; R; N; Q; S; G; W; | 2471-2475, 2477-2478, 2481- |
| | I; V; L; A;P | 2482, 2484-2489 |
| R189 | D; E; H; K; C; N; Q; T; Y; G; | 2490-2498, 2500, 2503, 2505- |
| | W; V; L; A; P | 2508 |
| E190 | D; H; K; R; C; T; Y; S; G; M; | 573-577, 580-583, 585, 587- |
| | I; V; L; A; P | 591 |
| Y191 | D; E; H; K; R; C; Q; T; S; G; | 592-597, 599-602, 605, 607- |
| | W; V; L; A; P | 610 |
| N192 | D; H; K; R; C; Q; T; S; G; M; | 611,613-618,620-621,623- |
| | W; V; L; A; P | 624, 626-629 |
| L193 | D; E; K; R; N; Q; T; Y; S; G; | 2509-2510, 2512-2513, 2515- |
| | F; W; I; A; P | 2521, 2523-2524, 2526-2527 |
| H194 | E; K; Q; T; Y; S; G; F; M; W; | 631-632, 636-648 |
| | I; V; L; A; P | |
| R195 | D; E; K; C; Q; T; Y; S; G; F; | 269-270, 272-273, 275-280, |
| | W; V; L; A; P | 282, 284-287 |
| V196 | D; E; H; K; R; Q; T; Y; S; G; | 2528-2532, 2535-2539, 2541, |
| | M; I; L; A; P | 2543-2546 |
| A197 | E; H; R; C; N; Q; T; Y; S; G; | 2548-2549, 2551-2558, 2561- |
| | W; I; V; L; P | 2565 |
| A198 | D; E; H; K; R; T; Y; S; G; F; | 288-292, 296-302, 304-306 |
| | M; W; V; L; P | |
| H199 | E; K; R; C; N; T; S; G; M; W; | 2567-2571, 2573, 2575-2576, |
| | I; V; L; A; P | 2578-2584 |
| E200 | D; R; C; N; T; Y; S; G; F; M; | 2585, 2588-2590, 2592-2600, |
| | W; I; V; A; P | 2602-2603 |
| L201 | D; E; K; R; N; Q; T; S; G; M; | 2604-2605, 2607-2608, 2610- |
| | W; I; V; A; P | 2612, 2614-2615, 2617-2622 |
| G202 | D; E; H; K; R; C; T; Y; S; M; | 2623-2628, 2631-2633, 2635, |
| | I; V; L; A; P | 2637-2641 |
| H203 | D; E; R; C; N; Q; T; Y; S; G; | 2642-2643, 2645-2652, 2656- |
| | I; V; L; A; P | 2660 |
| S204 | D; H; K; R; N; Q; T; Y; G; W; | 2661, 2663-2665, 2667-2671, |
| | I; V; L; A; P | 2674-2679 |
| L205 | D; E; R; C; N; Q; T; S; G; M; | 2680-2681-2684-2688, 2690- |
| | W; I; V; A; P | 2691, 2693-2698 |
| G206 | D; E; H; R; C; Q; T; S; M; W; | 307-309, 311-312, 314-315, |
| | I; V; L; A; P | 317, 319-325 |
| L207 | D; H; K; R; N; Q; Y; S; G; M; | 649, 651-653, 655-656, 658- |
| | W; I; V; A; P | 660, 662-667 |
| S208 | D; E; K; R; C; N; Q; T; C; F; | 2669-2700, 2702-2707, 2709- |
| | W; V; L; A; P | 2710, 2712, 2714-2717 |
| H209 | D; R; C; N; Q; T; Y; S; G; F; | 2718, 2721-2729, 2731, 2733- |
| | W; V; L; A; P | 2736 |
| S210 | H; K; R; C; N; Q; T; G; F; W; | 328-334, 336-337, 339-344 |
| | I; V; L; A; P | |
| T211 | D; H; K; R; N; Q; S; G; F; M; | 2737, 2739-2741, 2743-2744, |
| | W; V; L; A; P | 2746-2750, 2752-2755 |
| D212 | E; H; K; R; N; Q; T; Y; S; G; | |
| | F; V; L; A; P | 668-671, 673-679, 683-686 |
| I213 | D; E; H; K; R; C; N; Q; T; S; | 2756-2764, 2766-2769, 2771- |
| | G; F; M; V; L; A; P | 2774 |
| G214 | D; E; R; C; Q; T; Y; S; F; M; | 2775-2776, 2779-2780, 2782- |
| | I; V; L; A; P | 2787, 2789-2793 |
| A215 | D; H; K; R; C; N; Q; T; S; G; | 2794, 2796-2802, 2804-2805, |
| | M; W; I; V; L; P | 2807-2812 |
| L216 | D; E; K; R; C; Q; T; S; G; M; | 2813-2814, 2816-2818, 2820 |
| | W; I; V; A;P | 2821, 2823-2824, 2826-2831 |
| M217 | D; H; K; R; C; N; Q; T; Y; S; | 2832, 2834-2843, 2846, 2848- |
| | G; I; L; A; P | 2850 |
| Y218 | D; E; R; C; N; Q; S; G; F; W; | 345-346, 349-352, 354-356, |
| | I; V; L; A; P | 358-363 |
| P219 | D; E; H; K; R; C; Q; T; S; G; | 2851-2856, 2858-2859, 2861- |
| | F; W; V; L; A | 2863, 2865, 2867-2869 |
| S220 | E; H; K; R; N; Q; T; G; F; M; | 2871-2874, 2876-2878, 2880- |
| | I; V; L; A; P | 2882, 2884-2888 |
| Y221 | E; K; R; C; N; Q; T; S; G; M; | 2890, 2892-2899, 2901-2902, |
| | W; V; L; A; P | 2904-2907 |
| T222 | D; H; R; C; N; Y; S; C; F; M; | 2908, 2910, 2912-2914, 2916- |
| | W; I; V; L; A; P | 2926 |
| F223 | E; H; K; R; C; N; Q; T; Y; S; | 365-376, 380-382 |
| | G; M; L; A; P | |
| S224 | D; H; K; R; C; Q; T; G; M; W; | 2927, 2929-2932, 2934-2935, |
| | I; V; L; A; P | 2937, 293 9-2945 |
| G225 | D; E; H; K; R; C; N; Q; T; S; | 2946-2954, 2956, 2958-2959, |
| | M; W; V; A; P | 2961, 2963-2964 |
| D226 | E; H; R; C; N; T; S; C; M; W; | 2965-2966, 2968-2970, 2972, |
| | I; V; L; A; P | 2974-2975, 2977-2983 |
| V227 | D; E; H; K; R; C; Q; T; Y; S; | 383-388, 390-394, 397, 399- |
| | G; W; L; A; P | 401 |
| Q228 | D; E; H; K; R; N; T; Y; S; G; | 402-406, 408-412, 414-415, |
| | M; W; L; A; P | 418-420 |
| L229 | D; E; H; R; C; Q; T; Y; G; M; | 421-423, 425-426, 428-430, |
| | W; I; V; A; P | 432, 434-439 |
| A230 | D; H; R; C; N; T; Y; S; G; M; | 687, 689, 691-693, 695-698, |
| | W; I; V; L; P | 700-705 |
| Q231 | D; H; R; C; Y; S; G; F; M; W; | 2984, 2986, 2988-2989, 2992- |
| | I; V; L; A; P | 3002 |
| D232 | E; H; K; R; N; Q; T; Y; S; G; | 3003-3006, 3008-3014, 3016, |
| | F; W; V; L; P | 3018-3019, 3021 |
| D233 | E; K; R; N; Q; T; S; G; M; W; | 440, 442-443, 445-447, 449- |
| | I; V; L; A; P | 450, 452-458 |
| I234 | D; E; H; C; N; Q; T; Y; G; M; | 459-461, 464-468, 470, 472- |
| | W; V; L; A; P | 477 |
| D235 | E; H; R; C; N; Q; T; Y; S; G; | 3022-3023, 3025-3032, 3036- |
| | I; V; L; A; P | 3040 |
| G236 | D; E; K; R; C; N; T; Y; S; F; | 3041-3042, 3044-3 047, 3049- |
| | M; I; V; L; P | 3053, 3055-3057, 3059 |
| I237 | D; E; K; R; C; N; Q; T; Y; S; | 3060-3061, 3063-3071, 3074, |
| | G; W; L; A; P | 3076-3078 |
| Q238 | E; H; K; R; C; N; T; Y; S; G; | 3080-3090, 3092-3093, 3095, |
| | F; W; I; L; P | 3097 |
| A239 | D; H; K; R; C; Q; T; Y; S; G; | 3099, 3100-3103, 3105-3110, |
| | F; W; I; V; L; P | 3112-3116 |
| I240 | D; K; R; C; Q; T; Y; S; G; F; | 478, 481-483, 485-491, 493- |
| | M; V; L; A; P | 496 |
| Y241 | D; H; R; N; Q; T; S; G; M; W; | 3117, 3119, 3121, 3123-3127 |
| | I; V; L; A; P | 3129-3135 |
| G242 | E; H; K; R; N; T; Y; S; F; W; | 3137-3140, 3142, 3144-3147, |
| | I; V; L; A; P | 3149-3154 |
| R243 | D; H; K; C; N; Q; T; Y; S; G; | 3155, 3157-3165, 3169-3173 |
| | I; V; L; A; P | |
| S244 | D; E; H; R; Q; T; Y; C; F; M; | 3174-3176, 3178, 3181-3187, |
| | W; V; L; A; P | 3189-3192 |
| Q245 | E; H; K; R; C; T; S; G; F; M; | 3194-3198, 3200, 3202-3209, |
| | W; I; V; L; P | 3211 |
| N246 | D; K; R; C; Q; T; Y; S; G; F; | 3212, 3215-3223, 3225-3230 |
| | W; I; V; L; A; P | |
| P247 | D; E; H; K; R; N; Q; T; S; G; | 3231-3235, 3237-3239, 3241- |
| | F; I; V; L; A | 3243, 3246-3249 |
| V248 | E; H; K; R; C; Q; T; Y; S; G; | |
| | F; M; W; I; L; A | 3251-3255, 3257-3267 |
| Q249 | E; H; K; R; C; N; T; Y; G; W; | 3270-3277, 3279, 3282-3287 |
| | I; V; L; A; P | |
| P250 | D; K; R; N; Q; T; Y; S; G; F; | 3288, 3291-3292, 3294-3302, |
| | M; W; V; L; A | 3304-3306 |
| I251 | D; E; K; R; C; Q; T; Y; S; G; | 3307-3308, 3310-3312, 3314- |
| | W; V; L; A; P | 3318, 3321-3325 |
| G252 | D; E; H; K; R; C; T; S; F; M; | |
| | W; I; V; L; A; P | 3326-3331, 3334, 3336-3344 |
| P253 | E; K; R; C; N; Q; T; Y; G; M; | 3346, 3348-3354, 3356, 3358- |
| | W; I; V; L; A | 3363 |
| Q254 | D; E; R; C; T; Y; S; G; F; W; | 3364-3365, 3368-3369, 3371- |
| | I; V; L; A; P | 3375, 3377-3382 |
| T255 | E; H; K; R; C; N; Q; S; G; F; | 3384-3390, 3392-3394, 3397- |
| | I; V; L; A; P | 3401 |
| P256 | E; K; R; C; N; Q; Y; S; G; F; | 3403, 3405-3409, 3411-3415, |
| | M; I; V; L; A | 3417-3420 |
| K257 | E; R; C; N; T; S; G; F; M; W; | 3422, 3424-3426, 3428, 3430- |
| | I; V; L; A; P | 3439 |
| A258 | D; E; R; N; Q; T; Y; G; F; M; | 3440-3441, 3444, 3446-3449, |
| | W; I; V; L; P | 3451-3458 |

The cDNA encoding each individual hMMP-1 mutant was generated by changing the wildtype codon, encoding each of the 178 amino acids positions identified in Table 8 below, to a codon encoding the desired amino acid substitution. The wildtype codons are set forth in SEQ ID NO:706. SEQ ID NO:706 also depicts the encoded amino acids. The amino acids substitutions and corresponding mutated codons are listed in Table 8, below.

TABLE 8
Codons encoding each amino acid substitution

| Muta- | | Muta- | | Muta- | | Muta- | Co- |
|---|---|---|---|---|---|---|---|
| tion | Codon | tion | Codon | tion | Codon | tion | don |
| F81C | TGT | T84L | TTG | N87S | AGT | W90H | CAT |
| F81E | GAG | T84D | GAT | N87I | ATT | W90M | ATG |
| F81I | ATT | T84R | CGG | N87C | TGT | W90R | CGG |
| F81L | CTG | T84I | ATT | N87A | GCG | W90E | GAG |
| F81P | CCT | T84S | TCT | N87G | GGT | W90N | AAT |
| F81S | TCT | T84G | GGT | N87Y | TAT | W90Q | CAG |
| F81A | GCG | T84Q | GAG | N87E | GAG | E91N | AAT |
| F81M | ATG | T84P | CCT | N87H | CAT | E91R | CGG |
| F81G | GGG | T84A | GCG | N87Q | GAG | E91W | TGG |
| F81T | ACG | T84C | TGT | P88C | TGT | E91G | GGG |
| F81Q | GAG | T84V | TAT | P88K | AAG | E91V | GTG |
| F81R | CGT | T84F | TTT | P88W | TGG | E91Y | TAT |
| F81W | TGG | E85L | CTG | P88G | GGG | E91C | TGT |
| F81H | CAT | E8SQ | CAG | P88L | CTG | E91H | CAT |
| F81V | GTG | E85P | CCT | P88Q | GAG | E91T | ACG |
| V82I | ATT | E85T | ACT | P88A | GCG | E91S | AGT |
| V82C | TGT | E85K | AAG | P88T | ACG | E91A | GCG |
| V82A | GCG | E85M | ATG | P88Y | TAT | E91I | ATT |
| V82P | CCG | E85G | GGT | P88R | CGG | E91D | GAT |
| V82Y | TAT | E85R | CGT | P88H | CAT | E91F | TTT |
| V82M | ATG | E85S | TCT | P88I | ATT | E91L | TTG |
| V82Q | CAG | E85C | TGT | P88V | GTG | Q92V | GTT |
| V82F | TTT | E85Y | TAT | P88E | GAG | Q92Y | TAT |
| V82W | TGG | E85A | GCG | P88D | GAT | Q92L | CTG |
| V82N | AAT | E85N | AAT | R89V | GTG | Q92N | AAT |
| V82R | CGT | E8SV | GTG | R89W | TGG | Q92E | GAG |
| V82G | GGT | E85F | TTT | R89M | ATG | Q92I | ATT |
| V82S | TCG | GS6L | CTT | R89A | GCG | Q92T | ACT |
| V82L | TTG | G86P | CCG | R89T | ACG | Q92G | GGT |
| V82T | ACT | G86I | ATT | R89G | GGG | Q92P | CCG |
| L83A | GCG | G86T | ACT | R89S | TCT | Q92W | TGG |
| L83C | TGT | G86H | CAT | R89K | AAG | Q92F | TTT |
| L83D | CAT | G86D | GAT | R89F | TTT | Q92S | TCG |
| L83E | GAG | G86N | AAT | R89Y | TAT | Q92R | CGG |
| L83G | GGT | G86S | AGT | R89N | AAT | Q92K | AAG |
| L83H | CAT | G86K | AAG | R89H | CAT | Q92A | GCT |
| L83I | ATT | G86W | TGG | R89L | TTG | T93A | GCG |
| L83M | ATG | G86Y | TAT | R89E | GAG | T93L | CTT |
| L83P | CCG | G86V | GTT | R89P | CCT | T93M | ATG |
| L83Q | GAG | G86C | TGT | W90L | HG | T93N | AAT |
| L83R | CGG | G86M | ATG | W90G | GOG | T93V | GTG |
| L83S | AGT | G86F | TTT | W90P | CCG | T93I | ATT |
| L83T | ACG | N87M | ATG | W90T | ACT | T93D | GAT |
| L83W | TGG | N87L | CTG | W90S | TCG | T93S | TCG |
| L83Y | TAT | N87P | CGG | W90V | GTG | T93R | CGG |
| T84V | GTT | N87V | GTT | W90I | ATT | T93W | TGG |
| T84E | GAG | N87R | CGT | W90A | GCT | T93F | TTT |
| T84H | CAT | N87F | TTT | W90F | TTT | T93P | CCT |
| T93G | GGG | Y97R | CGT | E100L | CTG | T103R | CGG |
| T93K | AAG | Y97V | GTG | E100H | CAT | T103Y | TAT |
| T93E | GAG | Y97A | GCT | E100D | GAT | T103N | AAT |
| H94L | CTG | Y97P | CCT | E100M | ATG | T103C | TGT |
| H94S | TCG | Y97L | CTT | E100G | GGT | T103Q | CAG |
| H94M | ATG | Y97T | ACG | E100W | TGG | T103W | TGG |
| H94R | CGG | Y97K | AAG | E100Y | TAT | T103P | CCG |
| H94E | GAG | Y97W | TGG | E100R | CGT | T103A | GCG |
| H94I | ATT | Y97H | CAT | E100S | TCT | T103G | GGG |
| H94D | GAT | Y97S | TCG | E100T | ACG | T103K | AAG |
| H94P | CCG | Y97E | GAG | E100F | TTT | P104G | GGG |
| H94A | GCG | Y97D | GAT | E100T | ATT | P104E | GAG |
| H94N | AAT | Y97N | AAT | E100N | AAT | P104T | ACT |
| H94F | TTT | Y97G | GGT | N101M | ATG | P104F | TTT |
| H94G | GGG | Y97Q | CAG | N101F | TTT | P104R | CGT |
| H94T | ACT | R98H | CAT | N101L | TTG | P104D | GAT |
| H94V | GTG | R98K | AAG | N101Y | GTG | P104C | TGT |
| H94W | TGG | R9SC | TGT | N101H | CAT | P104Q | CAG |
| L95E | GAG | R98L | CTG | N101R | CGG | P104V | GTG |
| L9SY | TAT | R9SM | ATG | N101C | TGT | P104Y | TAT |
| L95R | CGG | R9SF | TTT | N101T | ACT | P104H | CAT |
| L95A | GCT | R98W | TGG | N101P | CCT | P104L | TTG |
| L95G | GGG | R98Y | TAT | N101W | TGG | P104S | TCG |
| L95K | AAG | R98P | CCT | N101K | AAG | P104A | GCG |
| L95S | AGT | R98E | GAG | N101S | TCG | P104M | ATG |
| L95T | ACG | R98A | GCG | N101D | GAT | D105A | GCT |
| L95H | CAT | R98G | GGG | N102A | GCG | D105C | TGT |
| L95W | TGG | R98V | GTT | N101Y | TAT | D105F | TTT |
| L95V | GTG | R98S | TCG | Y102R | CGT | D105G | GGT |
| L95C | TGT | R98D | GAT | Y102K | AAG | D105I | ATT |
| L95P | CCT | I99C | TGT | Y102V | GTG | D105L | CTG |
| L95D | GAT | I99E | GAG | Y102M | ATG | D105M | ATG |
| L95I | ATT | I99G | GGG | Y102P | CCG | D105N | AAT |
| T96E | GAG | I99H | CAT | Y102N | AAT | D105P | CCT |
| T96R | CGG | I99N | AAT | Y102G | GGG | D105R | CGG |
| T96P | CCG | I99P | CCT | Y102L | CTG | D105S | TCG |
| T96S | TCG | I99T | ACG | Y102D | GAT | D105T | ACG |
| T96A | GCG | I99V | GTT | Y102S | TCG | D105V | GTT |
| T96L | TTG | I99A | GCG | Y102F | TTT | D105W | TGG |
| T96W | TGG | I99F | TTT | Y102A | GCT | D105E | GAG |
| T96N | AAT | I99L | CTG | Y102E | GAG | L106P | CCG |
| T96G | GGT | I99R | CGT | Y102Q | CAG | L106D | GAT |
| T96F | TTT | I99S | TCG | Y102C | TGT | L106N | AAT |
| T96Q | CAG | I99Q | CAG | T103E | GAG | L106G | GGT |
| T96H | CAT | I99W | TGG | T103D | GAT | L106M | ATG |
| T96V | GTT | I99Y | TAT | T103S | AGT | L106A | GCT |
| T96I | ATT | E100V | GTT | T103L | CTG | L106R | CGG |
| T96C | TGT | E100P | CCG | T103V | GTT | L106Y | TAT |
| L106T | ACG | A109V | GTT | D112I | ATT | E116A | GCG |
| L106V | GTG | A109E | GAG | D112Y | TAT | E116C | TGT |
| L106H | CAT | A109L | CTT | D112L | TTG | E116D | GAT |
| L106F | TTT | A109H | CAT | H113T | ACT | E116F | TTT |
| L106I | ATT | D110P | CCT | H113L | CTG | E116G | GGT |
| L106C | TGT | D110F | TTT | H113M | ATG | E116H | CAT |
| L106S | TCT | D110Q | CAG | H113S | TCG | E116I | ATT |
| P107L | TTG | D110R | CGG | H113N | AAT | E116K | AAG |
| P107W | TGG | D110M | ATG | H113R | AGG | E116L | CTG |
| P107T | ACT | D110H | CAT | H113A | GCT | E116M | ATG |
| P107S | TCG | D110I | ATT | H113E | GAG | E116N | AAT |
| P107R | CGG | D110L | CTT | H113V | GTG | E116P | CCG |
| P107Y | TAT | D110V | GTG | H113Y | TAT | E116Q | CAG |
| P107M | ATG | D110T | ACG | H113F | TTT | E116R | AGG |
| P107V | GTG | D110S | TCG | H113D | GAT | E116S | TCT |
| P107D | GAT | D110Y | TAT | H113W | TGG | K117H | CAT |
| P107A | GCG | D110G | GGT | H113G | GGG | K117T | ACG |
| P107C | TGT | D110C | TGT | H113P | CCG | K117Q | CAG |
| P107K | AAG | D110A | GCG | A114E | GAG | K117E | GAG |
| P107F | TTT | V111E | GAG | A114S | TCG | K117A | GCG |
| P107I | ATT | V111A | GCT | A114I | ATT | K117F | TTT |
| P107G | GGT | V111S | TCT | A114P | CCT | K117D | GAT |
| R108P | CCT | V111W | TGG | A114N | AAT | K117N | AAT |
| R108G | GGT | V111G | GGT | A114L | CTT | K117G | GGT |
| R108T | ACG | V111Y | TAT | A114T | ACT | K117W | TGG |
| R108E | GAG | V111P | CCG | A114F | TTT | K117Y | TAT |
| R108A | GCG | V111L | CTG | A114V | GTT | K117L | TTG |
| R108Y | TAT | V111D | GAT | A114G | GGT | K117S | AGT |
| R108K | AAG | V111K | AAG | A114C | TGT | K117P | CCG |
| R108C | TGT | V111T | ACT | A114M | ATG | K117R | AGG |
| R108S | TCT | V111Q | CAG | A114R | AGG | A118G | GGG |
| R108F | TTT | V111I | ATT | A114W | TGG | A118R | CGT |
| R108W | TGG | V111C | TGT | A114Q | CAG | A118W | TGG |
| R108I | ATT | V111R | CGT | I115F | TTT | A118K | AAG |
| R108L | CTT | D112A | GCG | I115T | ACT | A118P | CCT |
| R108N | AAT | D112M | ATG | I115H | CAT | A118V | GTG |
| R108V | GTT | D112V | GTT | I115G | GGT | A118L | TTG |
| A109S | TCG | D112R | CGG | I115K | AAG | A118D | GAT |
| A109R | CGG | D112K | AAG | I115E | GAG | A118S | AGT |
| A109T | ACG | D112P | CCT | I1155 | AGT | A118F | TTT |
| A109W | TGG | D112Q | CAG | I115P | CCT | A118I | ATT |
| A109I | ATT | D112F | TTT | I115C | TGT | A118H | CAT |
| A109Q | CAG | D112G | GGG | I115L | CTT | A118E | GAG |
| A109N | AAT | D112C | TGT | I115Q | CAG | A118Q | CAG |
| A109Y | TAT | D112W | TGG | I115R | CGG | A118T | ACT |
| A109G | GGG | D112T | ACT | I115W | TGG | F119G | GGG |
| A109M | ATG | D112H | CAT | I115V | GTT | F119T | ACT |
| A109D | GAT | D112S | TCT | I115D | GAT | F119R | CGG |
| F119L | TTG | W122G | GGG | V125T | ACG | L128A | GCG |
| F119N | AAT | W122S | TCG | V125A | GCT | L128D | GAT |
| F119S | AGT | W122V | GTT | V125C | TGT | L128V | GTG |
| F119C | TGT | W122H | CAT | V125D | GAT | L128W | TGG |
| F119P | CCG | W122F | TTT | V125W | TGG | L128C | TGT |
| F119W | TGG | W122Y | TAT | V125R | CGG | L128K | AAG |
| F119K | AAG | W122K | AAG | V125E | GAA | T129G | GGT |
| F119H | CAT | W122Q | CAG | V125F | TTT | T129A | GCT |
| F119A | GCG | W122E | GAG | V125H | CAT | T129C | TGT |
| F119V | GTT | S123D | GAT | T126K | AAG | T129K | AAG |
| F119Y | TAT | S123L | TTG | T126V | GTG | T129F | TTT |
| F119E | GAG | S123A | GCT | T126G | GGG | T129Y | TAT |
| Q120K | AAG | S123C | TGT | T126R | CGG | T129S | TCG |
| Q120N | AAT | S123I | ATT | T126L | TTG | T129R | CGC |
| Q120A | GCG | S123K | AAG | T126H | CAT | T129V | GTT |
| Q120V | GTG | S123N | AAT | T126M | ATG | T129L | CTT |
| Q120D | GAT | S123F | TTT | T126P | CCG | T129H | CAT |
| Q120R | CGG | S123Y | TAT | T126A | GCG | T129P | CCT |
| Q120P | CCT | S123M | ATG | T126N | AAT | T129E | GAG |
| Q120W | TGG | S123H | CAT | T126E | GAG | T129I | ATT |
| Q120Y | TAT | S123R | CGG | T126F | TTT | T129M | ATG |
| Q120C | TGT | S123W | TGG | T126W | TGG | F130L | CTG |
| Q120H | CAT | S123T | ACG | T126Q | CAG | F130P | CCT |
| Q120T | ACT | S123P | CCT | T126S | AGT | F130C | TGT |
| Q120M | ATG | S123G | GGG | P127C | TGT | F130R | CGG |
| Q120E | GAG | S123Q | CAG | P127F | TTT | F130Y | TAT |
| Q120G | GGT | S123V | GTT | P127T | ACG | F130H | CAT |
| L121E | GAG | N124G | GGT | P127E | GAG | F130I | ATT |
| L121Q | CAG | N124C | TGT | P127W | TGG | F130V | GTT |
| L121P | CCT | N124V | GTG | P127A | GCT | F130K | AAC |
| L121R | CGG | N124L | CTT | P127S | AGT | F130T | ACT |
| L121C | TGT | N124T | ACG | P127H | CAT | F130E | GAG |
| L121G | GGG | N124R | CGT | P127Q | CAG | F130A | GCG |
| L121K | AAG | N124M | ATG | P127K | AAG | F130N | AAT |
| L121F | TTT | N124S | TCG | P127R | CGG | F130G | GGT |
| L121I | ATT | N124P | CCT | P127I | ATT | F130S | AGT |
| L121S | TCG | N124A | GCG | P127V | GTG | T131F | TTT |
| L121V | GTT | N124K | AAG | P127L | CTG | T131P | CCC |
| L12IH | CAT | N124F | TTT | P127M | ATG | T131A | GCG |
| L121T | ACT | N124W | TGG | L128F | TTT | T131S | TCT |
| L121A | GCT | N124I | ATT | L128M | ATG | T131G | GGT |
| L121N | AAT | N124D | GAT | L128T | ACT | T131I | ATT |
| W122R | CGT | V125G | GGG | L128R | CGT | T131L | CTT |
| W122A | GCG | V125Q | CAG | L128S | TCG | T131H | CAT |
| W122N | AAT | V125S | TCG | L128G | GGT | T131Q | CAG |
| W122P | CCC | V125P | CCG | L128I | ATT | T131D | GAT |
| W122T | ACG | V125M | ATG | L128Q | CAG | T131E | GAG |
| W122L | CTT | V125Y | TAT | L128P | CCT | T131C | TGT |
| T131R | CGT | E135V | GTT | A138C | TGT | M141S | AGT |
| T131Y | TAT | E135M | ATG | A138T | ACG | M141C | TGT |
| T131M | ATG | E13SS | TCG | A138S | TCT | M141L | CTG |
| K132G | GGT | E135D | GAT | A138R | CGT | M141A | GCG |
| K132V | GTG | E135T | ACG | A138G | GGG | M141D | GAT |
| K132L | TTG | E135L | CTG | A138E | GAG | M141W | TGG |
| K132A | GCT | E135A | GCG | A138H | CAT | M141G | GGT |
| K132P | CCG | E135W | TGG | A138M | ATG | M141H | CAT |
| K132F | TTT | E135F | TTT | A138Q | CAG | M141Y | TAT |
| K132R | CGG | E135P | CCG | A138T | ATT | M141N | AAT |
| K132I | ATT | E135R | CGG | A138D | GAT | I142L | CTG |
| K132H | CAT | E135N | AAT | A138W | TGG | I142M | ATG |
| K132S | TCT | E135H | CAT | D139R | CGT | I142G | GGT |
| K132M | ATG | E135Q | CAG | D139V | GTT | I142K | AAG |
| K132D | GAT | E135I | ATT | D139M | ATG | I142A | GCT |
| K132T | ACT | G136V | GTG | D139C | TGT | I142N | AAT |
| K132Y | TAT | G136W | TGG | D139P | CCT | I142W | TGG |
| K132E | GAG | G136D | GAT | D139S | TCT | I142P | CCG |
| V133G | GGG | G136M | ATG | D139L | CTT | I142Q | CAG |
| V133E | GAG | G136N | AAT | D139I | ATT | I142Y | TAT |
| V133T | ACT | G136A | GCG | D139H | CAT | I142V | GTG |
| V133N | AAT | G136L | TTG | D139A | GCG | I142T | ACT |
| V133A | GCG | G136C | TGT | D139G | GGG | I142R | CGG |
| V133H | CAT | G136P | CCG | D139F | TTT | I142S | AGT |
| V133P | CCG | G136T | ACG | D139N | AAT | I142F | TTT |
| V133K | AAG | G136R | CGT | D139W | TGG | S143P | CCG |
| V133R | CGG | G136S | TCG | D139Y | TAT | S143C | TGT |
| V133L | CTT | G136I | ATT | D139E | GAG | S143E | GAG |
| V133W | TGG | G136H | CAT | I140D | GAT | S143G | GGT |
| V133C | TGT | G136E | GAG | I140K | AAG | S143H | CAT |
| V133D | GAT | Q137A | GCT | I140A | GCT | S143R | CGT |
| V133M | ATG | Q137R | CGG | I140G | GGG | S143L | TTG |
| V133S | AGT | Q137Q | GGG | I140C | TGT | S143Q | GAG |
| S134V | GTT | Q137K | AAG | I140Y | TAT | S143N | AAT |
| S134H | CAT | Q137H | CAT | I140V | GTT | S143W | TGG |
| S134P | CCT | Q137P | CCT | I140W | TGG | S143A | GCT |
| S134G | GGG | Q137S | TCG | I140F | TTT | S143T | ACT |
| S134N | AAT | Q137L | CTG | I140H | CAT | S143Y | TAT |
| S134R | CGT | Q137W | TGG | I140L | CTG | S143M | ATG |
| S134L | CTG | Q137F | TTT | I140R | CGG | S143I | ATT |
| S134Q | CAG | Q137T | ACG | I140E | GAG | F144K | AAG |
| S134E | GAG | Q137C | TGT | I140M | ATG | F144M | ATG |
| S134Y | TAT | Q137Y | TAT | I140T | ACT | F144E | GAG |
| S134A | GCG | Q137N | AAT | M141E | GAG | F144S | AGT |
| S134K | AAG | Q137E | GAG | M141I | ATT | F144L | CTG |
| S134D | GAT | A138V | GTT | M141R | CGG | F144W | TGG |
| S134T | ACG | A138L | CTT | M141T | ACG | F144P | CCG |
| S134C | TGT | A138P | CCG | M141P | CCG | F144R | CGG |
| F144N | AAT | G147V | GTT | R150H | CAT | P154L | CTT |
| F144C | TGT | G147Q | CAG | D151R | CGT | P154C | TGT |
| F144G | GGT | G147M | ATG | D151F | TTT | P154S | TCT |
| F144T | ACT | G147P | CCT | D151P | CCG | P154K | AAG |
| F144Q | CAG | D148R | CGG | D151W | TGG | P154I | ATT |
| F144H | CAT | D148I | ATT | D151Q | GAG | P154A | GCT |
| F144V | GTG | D148T | ACG | D151L | CTT | P154T | ACG |
| V145A | GCG | D148G | GGT | D151S | TCG | P154H | CAT |
| V145T | ACG | D148L | CTG | D151G | GGT | P154Y | TAT |
| V145L | CTG | D148V | GTT | D151A | GCT | P154N | AAT |
| V145P | CCG | D148A | GCG | D151N | AAT | P154F | TTT |
| V145K | AAG | D148W | TGG | D151K | AAG | P154R | CGT |
| V145N | AAT | D148P | CCG | D151Y | TAT | P154Q | CAG |
| V145D | GAT | D148S | TCG | D151V | GTT | F155S | TCT |
| V145H | CAT | D148K | AAG | D151T | ACT | F155T | ACT |
| V145R | CGG | D148E | GAG | D151M | ATG | F155G | GGT |
| V145Q | CAG | D148M | ATG | N152G | GGG | F155N | AAT |
| V145S | TCT | D148N | AAT | N152C | TGT | F155R | CGG |
| V145G | GGG | D148C | TGT | N152F | TTT | F155W | TGG |
| V145W | TGG | H149W | TGG | N152L | TTG | F155L | CTG |
| V145C | TGT | H149A | GCG | N152P | CCG | F155Q | CAG |
| V145E | GAG | H149L | TTG | N152R | CGG | F155M | ATG |
| R146T | ACG | H149C | TGT | N152H | CAT | F155E | GAG |
| R146L | CTG | H149Q | CAG | N152T | ACG | F155A | GCG |
| R146N | AAT | H149T | ACT | N152Y | TAT | F155P | CCT |
| R146H | CAT | H149Y | TAT | N152K | AAG | F155V | GTT |
| R146Q | CAG | H149P | CCG | N152D | GAT | F155H | CAT |
| R146K | AAG | H149V | GTT | N152W | TGG | F155Y | TAT |
| R146C | TGT | H149R | CGG | N152I | ATT | D156H | CAT |
| R146S | AGT | H149G | GGT | N152A | GCG | D156L | CTT |
| R146D | GAT | H149E | GAG | N152S | TCT | D156E | GAG |
| R146A | GCT | H149S | AGT | S153I | ATT | D156A | GCT |
| R146Y | TAT | H149I | ATT | S153R | CGG | D156W | TGG |
| R146P | CCT | H149N | AAT | S153K | AAG | D156C | TGT |
| R146V | GTT | R150S | TCG | S153C | TGT | D156P | CCT |
| R146E | GAG | R150E | GAG | S153G | GGG | D156V | GTT |
| R146F | TTT | R150G | GGG | S153H | CAT | D156K | AAG |
| G147R | CGT | R150M | ATG | S153L | CTT | D156S | TCT |
| G147F | TTT | R150P | CCC | S153V | GTT | D156G | GGG |
| G147I | ATT | R150T | ACG | S153T | ACG | D156T | ACT |
| G147L | CTG | R150W | TGG | S153P | CCT | D156Y | TAT |
| G147A | GCG | R150A | GCG | S153A | GCG | D156R | CGT |
| G147E | GAG | R150N | AAT | S153F | TTT | D156M | ATG |
| G147H | CAT | R150K | AAG | S153D | GAT | G157K | AAG |
| G147W | TGG | R150L | TTG | S153Q | CAG | G157D | GAT |
| G147T | ACG | R150V | GTT | S153Y | TAT | G157F | TTT |
| G147C | TGT | R150D | GAT | P154V | GTT | G157R | CGT |
| G147S | TCT | R150I | ATT | P154W | TGG | G157H | CAT |
| G157L | TTG | G160M | ATG | A163E | GAG | F166C | TGT |
| G157N | AAT | G160C | TGT | A163T | ACG | F166E | GAG |
| G157Y | TAT | G160Q | CAG | A163Q | CAG | Q167D | GAT |
| G157S | TCG | G160V | GTT | A163I | ATT | Q167R | CGG |
| G157T | ACG | G160S | AGT | A163N | AAT | Q167A | GCG |
| G157A | GCT | G160E | GAG | H164L | CTT | Q167S | AGT |
| G157Q | CAG | G160L | CTT | H164M | ATG | Q167F | TTT |
| G157P | CCG | G160T | ACG | H164K | AAG | Q167Y | TAT |
| G157V | GTG | N161S | AGT | H164P | CCG | Q167P | CCG |
| G157M | ATG | N161C | TGT | H164C | TGT | Q167T | ACT |
| P158S | TCT | N161L | TTG | H164R | CGT | Q167V | GTG |
| P158Y | TAT | N161R | CGT | H164A | GCG | Q167L | CTG |
| P158R | CGG | N161G | GGT | H164V | GTG | Q167M | ATG |
| P158L | CTT | N161W | TGG | H164S | TCG | Q167N | AAT |
| P158V | GTG | N161Y | TAT | H164N | AAT | Q167G | GGG |
| P158C | TGT | N161E | GAG | H164G | GGG | Q167K | AAG |
| P158A | GCG | N161P | CCT | H164F | TTT | Q167E | GAG |
| P158W | TGG | N161T | ACG | H164Y | TAT | P168N | AAT |
| P158I | ATT | N161H | CAT | H164Q | CAG | P168F | TTT |
| P158F | TTT | N161I | ATT | H164E | GAG | P168R | CGG |
| P158Q | CAG | N161V | GTG | A165W | TGG | P168W | TGG |
| P158T | ACT | N161F | TTT | A165V | GTT | P168A | GCT |
| P158G | GGT | N161Q | CAG | A165G | GGG | P168T | ACG |
| P158K | AAG | L162A | GCT | A165K | AAG | P168V | GTT |
| P158N | AAT | L162G | GGG | A165L | TTG | P168G | GGG |
| P158D | GAT | L162C | TGT | A16SP | CCT | P168C | TGT |
| G159R | CGG | L162P | CCG | A165Q | CAG | P168M | ATG |
| G159S | AGT | L162R | CGG | A165D | GAT | P168H | CAT |
| G159Q | CAG | L162I | ATT | A165H | CAT | P168L | CTT |
| G159P | CCT | L162S | TCT | A165F | TTT | P168S | AGT |
| G159V | GTG | L162D | GAT | A165S | AGT | P168I | ATT |
| G159K | AAG | L162M | ATG | A165T | ACT | P168D | GAT |
| G159A | GCG | L162E | GAG | A165R | CGG | G169H | CAT |
| G159Y | TAT | L162T | ACT | A165N | AAT | G169A | GCG |
| G159E | GAG | L162Y | TAT | A165M | ATG | G169E | GAG |
| G159T | ACG | L162F | TTT | F166G | GGG | G169C | TGT |
| G159M | ATG | L162W | TGG | F166S | TCG | G169S | TCG |
| G159I | ATT | L162Q | CAG | F166L | CTT | G169L | CTG |
| G159W | TGG | A163R | CGT | F166V | GTG | G169V | GTT |
| G159L | CTG | A163G | GGG | F166P | CCT | G169T | ACG |
| G159C | TGT | A163Y | TAT | F166N | AAT | G169R | CGG |
| G160A | GCG | A163P | CCT | F166R | CGT | G169W | TGG |
| G160H | CAT | A163S | AGT | F166A | GCG | G169M | ATG |
| G160N | AAT | A163L | CTT | F166K | AAG | G169I | ATT |
| G160W | TGG | A163C | TGT | F166H | CAT | G169P | CCG |
| G160R | CGG | A163K | AAG | F166W | TGG | G169D | GAT |
| G160P | CCG | A163V | GTG | F166I | ATT | G169Q | CAG |
| G160I | ATT | A163F | TTT | F166M | ATG | P170L | CTT |
| P170R | CGG | G173S | AGT | A176L | CTG | D179I | ATT |
| P170I | ATT | G173A | GCG | A176P | CCT | D179R | CGT |
| P170T | ACG | G173R | AGG | A176N | AAT | D179N | AAT |
| P170F | TTT | G173N | AAT | A176G | GGT | D179W | TGG |
| P170Q | CAG | G173T | ACG | A176S | TCT | D179Q | CAG |
| P170G | GGG | G173D | GAT | A176R | CGT | D179V | GTG |
| P170S | TCT | G173V | GTT | A176K | AAG | D179C | TGT |
| P170H | CAT | G173F | TTT | A176D | GAT | E180M | ATG |
| P170C | TGT | G173M | ATG | A176W | TGG | E180P | CCT |
| P170M | ATG | G173Y | TAT | H177T | ACG | E180K | AAG |
| P170K | AAG | G173P | CCG | H177P | CCG | E180Y | TAT |
| P170W | TGG | G174R | CGT | H177Q | CAG | E180Q | CAG |
| P170D | GAT | G174A | GCG | H177A | GCG | E180R | CGG |
| P170A | GCG | G174E | GAG | H177S | TCG | E180A | GCG |
| G171S | TCT | G174F | TTT | H177G | GGG | E180T | ACT |
| G171M | ATG | G174H | CAT | H177W | TGG | E180I | ATT |
| G171N | AAT | G174T | ACT | H177L | CTG | E180F | TTT |
| G171P | CCT | G174D | GAT | H177V | GTT | E180C | TGT |
| G171R | CGG | G174S | AGT | H177I | ATT | E180G | GGG |
| G171Y | TAT | G174P | CCG | H177R | CGG | E180S | TCG |
| G171A | GCT | G174W | TGG | H177N | AAT | E180N | AAT |
| G171Q | CAG | G174V | GTT | H177Y | TAT | E180D | GAT |
| G171H | CAT | G174N | AAT | H177C | TGT | D181S | TCG |
| G171L | CTT | G174Y | TAT | H177D | GAT | D181Q | GAG |
| G171W | TGG | G174M | ATG | F178G | GGT | D181P | CCT |
| G171C | TGT | G174L | CTT | F178C | TGT | D181Y | TAT |
| G171K | AAG | D175I | ATT | F178W | TGG | D181R | CGT |
| G171E | GAG | D175T | ACG | F178R | CGG | D181V | GTT |
| G171D | GAT | D175N | AAT | F178K | AAG | D181F | TTT |
| I172Y | TAT | D175V | GTT | F178S | AGT | D181A | GCT |
| I172T | ACG | D175S | TCG | F178H | CAT | D181T | ACG |
| I172P | CCT | D175R | CGG | F178P | CCT | D181L | TTG |
| I172A | GCG | D175G | GGG | F178V | GTT | D18IE | GAG |
| I172L | CTT | DI75A | GCG | F178A | GCT | D181K | AAG |
| I172Q | CAG | D175F | TTT | F178Q | CAG | D181M | ATG |
| I172E | GAG | D175C | TGT | F178Y | TAT | D181C | TGT |
| I172C | TGT | D175Q | CAG | F178I | ATT | D181G | GGT |
| I172M | ATG | D175Y | TAT | F178T | ACT | E182C | TGT |
| I172D | GAT | D175L | CTG | F178L | CTG | E182P | CCT |
| I172V | GTT | D175H | CAT | F178E | GAG | E182S | AGT |
| I172R | CGT | D175P | CCG | D179P | CCT | E182T | ACC |
| I172G | GGG | D175E | GAG | D179L | TTG | E182R | CGG |
| I172W | TGG | A176F | TTT | D179E | GAG | E182D | GAT |
| I172N | AAT | A176Q | CAG | D179G | GGG | E182A | GCT |
| G173C | TGT | A176V | GTG | D179S | AGT | E182F | TTT |
| G173L | CTG | A176E | GAG | D179A | GCT | E182L | CTT |
| G173K | AAG | A176T | ACT | D179K | AAG | E182I | ATT |
| G173W | TGG | A176C | TGT | D179T | ACT | E182Y | TAT |
| E182Q | CAG | T185D | GAT | R189K | AAG | N192S | TCG |
| E182W | TGG | N186G | GGG | R189P | CCG | N192W | TGG |
| E182M | ATG | N186A | GCT | R189E | GAG | N192G | GGG |
| E182G | GGT | N186T | ACT | R189V | GTT | N192D | CAT |
| R183P | CCT | N186R | CGT | R189D | GAT | N192V | GTG |
| R183K | AAG | N186L | TTG | R189Y | TAT | N192A | GCT |
| R183W | TGG | N186P | CCG | R189C | TGT | N192T | ACT |
| R183E | GAG | N186S | AGT | R189A | GCT | N192K | AAG |
| R183A | GCT | N186V | GTG | R189H | CAT | N192C | TGT |
| R183T | ACG | N186Q | CAG | R189W | TGG | N192M | ATG |
| R183L | CTT | N186H | CAT | R189N | AAT | L193P | CCG |
| R183N | AAT | N186C | TGT | R189T | ACT | L193G | GGG |
| R183H | CAT | N186E | GAG | R189Q | CAG | L193F | TTT |
| R183V | GTG | N186F | TTT | E190A | GCG | L193S | TCG |
| R183C | TGT | N186Y | TAT | E190H | CAT | L193W | TGG |
| R183M | ATG | N186D | GAT | E190V | GTG | L193A | GCT |
| R183I | ATT | N187R | CGG | E190P | CCC | L193R | CGT |
| R183G | GGT | N187M | ATG | E190C | TGT | L193Q | CAG |
| R183S | TCT | N187S | TCT | E190G | GGT | L193E | GAG |
| W184G | GGG | N187T | ACG | E190R | CGG | L193K | AAG |
| W184H | CAT | N187L | CTG | E190I | ATT | L193N | AAT |
| W184L | CTG | N187W | TGG | E190S | TCG | L193I | ATT |
| W184E | GAG | N187F | TTT | E190T | ACT | L193T | ACT |
| W184P | CCT | N187K | AAG | E190M | ATG | L193D | CAT |
| W184N | AAT | N187I | ATT | E190L | TTG | L193Y | TAT |
| W184A | GCG | N187A | GCT | E190K | AAG | H194S | AGT |
| W184T | ACT | N187P | CCG | E190Y | TAT | H194E | GAG |
| W184R | CGG | N187D | GAT | E190D | GAT | H194K | AAG |
| W184Q | CAG | N187G | GGG | Y191T | ACT | H194Q | CAG |
| W184V | GTG | N187C | TGT | Y191H | CAT | H194V | GTT |
| W184S | TCT | N187H | CAT | Y191G | GGG | H194T | ACT |
| W184M | ATG | F188P | CCG | Y191L | TTG | H194L | CTG |
| W184I | ATT | F188I | ATT | Y191P | CCT | H194Y | TAT |
| W184F | TTT | F188N | AAT | Y191Q | CAG | H194F | TTT |
| T185R | CGT | F188S | AGT | Y191K | AAG | H194G | GGT |
| T185Y | TAT | F188Q | CAG | Y191D | GAT | H194I | ATT |
| T185W | TGG | F188K | AAG | Y191A | GCG | H194W | TGG |
| T185H | CAT | F188G | GGG | Y191W | TGG | H194M | ATG |
| T185G | GGG | F188W | TGG | Y191S | TCT | H194A | GCT |
| T185P | CCT | F188E | GAG | Y191V | GTT | H194P | CCT |
| T185S | TCG | F188H | CAT | Y191E | GAG | R195C | TGT |
| T185V | GTT | F188D | GAT | Y191R | CGT | R195F | TTT |
| T185Q | CAG | F188A | GCG | Y191C | TGT | R195W | TGG |
| T185N | AAT | F188L | CTT | N192R | CGG | R195T | ACT |
| T185C | TGT | F188R | CGT | N192L | CTG | R195L | CTG |
| T185L | CTT | F188V | GTT | N192Q | CAG | R195G | GGT |
| T185A | GCG | R189L | TTG | N192P | CCT | R195Q | CAG |
| T185E | GAG | R189G | GGG | N192H | CAT | R195K | AAG |
| R195S | TCT | A198F | TTT | L201N | AAT | L205S | TCT |
| R195A | GCT | A198W | TGG | G202T | ACG | L205G | GGT |
| R195D | GAT | A198Y | TAT | G202Y | TAT | L205P | CCT |
| R195P | CCT | A198D | GAT | G202E | GAG | L205E | GAG |
| R195Y | TAT | H199I | ATT | G202V | GTG | L205V | GTG |
| R195E | GAG | H199P | CCG | G202S | TCT | L205M | ATG |
| R195V | GTG | H199G | GGT | G202L | CTG | L205N | AAT |
| V196T | ACG | H199N | AAT | G202I | ATT | L205C | TGT |
| V196D | GAT | H199S | TCG | G202M | ATG | L205I | ATT |
| V196G | GGG | H199L | TTG | G202H | CAT | L205A | GCG |
| V196E | GAG | H199M | ATG | G202C | TGT | L205R | CGG |
| V196A | GCG | H199A | GCG | G202R | CGT | L205W | TGG |
| V196S | AGT | H199C | TGT | G202P | CCT | L205Q | CAG |
| V196Q | CAG | H199K | AAG | G202A | GCT | G206I | ATT |
| V196P | CCG | H199R | CGT | G202K | AAG | G206V | GTG |
| V196R | CGT | H199V | GTG | G202D | GAT | G206A | GCG |
| V196H | CAT | H199W | TGG | H203Y | TAT | G206C | TGT |
| V196Y | TAT | H199T | ACT | H203E | GAG | G206S | TCG |
| V196I | ATT | H199E | GAG | H203R | CGG | G206P | CCG |
| V196L | CTG | E200P | CCG | H203Q | CAG | G206L | TTG |
| V196K | AAG | E200G | GGG | H203P | CCG | G206D | GAT |
| V196M | ATG | E200A | GCT | H203G | GGG | G206M | ATG |
| A197G | GGT | E200T | ACG | H203T | ACT | G206R | CGG |
| A197S | AGT | E200I | ATT | H203D | GAT | G206Q | CAG |
| A197L | CTT | E200W | TGG | H203L | TTG | G206E | GAG |
| A197P | CCG | E200R | CGG | H203N | AAT | G206H | CAT |
| A197V | GTG | E200F | TTT | H203A | GCT | G206T | ACG |
| A197Y | TAT | E200M | ATG | H203S | TCT | G206W | TGG |
| A197Q | CAG | E200D | GAT | H203V | GTT | L207S | TCT |
| A197R | CGG | E200V | GTG | H203I | ATT | L207Y | TAT |
| A197T | ACT | E200C | TGT | H203C | TGT | L207A | GCG |
| A197I | ATT | E200S | TCT | S204R | CGG | L207R | CGT |
| A197H | CAT | E200Y | TAT | S204N | AAT | L207P | CCG |
| A197E | GAG | E200N | AAT | S204A | GCG | L207Q | CAG |
| A197W | TGG | L201A | GCG | S204T | ACT | L207N | AAT |
| A197N | AAT | L201R | CGG | S204Y | TAT | L207K | AAG |
| A197C | TGT | L201E | GAG | S204V | GTG | L207M | ATG |
| A198T | ACG | L201P | CCT | S204L | CTT | L207W | TGG |
| A198K | AAG | L201G | GGT | S204H | CAT | L207H | CAT |
| A198S | TCG | L201V | GTT | S204D | GAT | L207D | GAT |
| A198H | CAT | L201T | ACG | S204Q | CAG | L207V | GTT |
| A198G | GGT | L201I | ATT | S204G | GGG | L207I | ATT |
| A198E | GAG | L201S | TCT | S204W | TGG | L207G | GGT |
| A198P | CCG | L201W | TGG | S204I | ATT | S208D | GAT |
| A198L | TTG | L201Q | CAG | S204K | AAG | S208V | GTT |
| A198R | CGT | L201D | GAT | S204P | CCT | S208P | CCT |
| A198V | GTT | L201M | ATG | L205T | ACG | S208G | GGT |
| A198M | ATG | L201K | AAG | L205D | GAT | S208A | GCG |
| S208K | AAG | T211Q | CAG | G214A | GCT | M217A | GCG |
| S208N | AAT | T211S | TCG | G214D | GAT | M217H | CAT |
| S208F | TTT | T211A | GCG | G214F | TTT | M217I | ATT |
| S208Q | CAG | T211F | TTT | G214Y | TAT | M217D | GAT |
| S208W | TGG | T211D | GAT | G214M | ATG | Y218C | TGT |
| S20ST | ACG | T211W | TGG | G214C | TGT | Y218F | TTT |
| S208E | GAG | T211L | CTG | A215L | CTG | Y218W | TGG |
| S208C | TGT | D212E | GAG | A215Q | CAG | Y218L | CTG |
| S208R | CGT | D212A | GCG | A215M | ATG | Y218A | GCG |
| S208L | CTT | D212K | AAG | A215G | GGT | Y218P | CCG |
| H209T | ACG | D212R | CGG | A215W | TGG | Y218R | CGG |
| H209Y | TAT | D212T | ACG | A215S | AGT | Y218N | AAT |
| H209R | CGG | D212N | AAT | A215T | ACG | Y218V | GTG |
| H209Q | CAG | D212G | GGG | A215V | GTT | Y218Q | CAG |
| H209A | GCT | D212S | TCT | A215N | AAT | Y218I | ATT |
| H209G | GGG | D212P | CCG | A215P | CCG | Y218D | GAT |
| H209N | AAT | D212Q | CAG | A215H | CAT | Y218S | TCG |
| H209P | CCT | D212V | GTT | A215K | AAG | Y218G | GGG |
| H209W | TGG | D212L | TTG | A215I | ATT | Y218E | GAG |
| H209V | GTT | D212F | TTT | A215R | CGT | P219L | TTG |
| H209D | GAT | D212H | CAT | A215C | TGT | P219C | TGT |
| H209S | AGT | D212Y | TAT | A215D | GAT | P219V | GTG |
| H209F | TTT | I213Q | CAG | L216A | GCT | P219D | GAT |
| H209L | CTG | I213T | ACT | L216C | TGT | P219F | TTT |
| H209C | TGT | I213C | TGT | L216D | GAT | P219A | GCG |
| S210C | TGT | I213P | CCT | L216E | GAG | P219T | ACT |
| S210G | GGT | I213H | CAT | L216G | GGG | P219E | GAG |
| S210I | ATT | I213A | GCG | L216I | ATT | P219Q | CAG |
| S210R | CGT | I213V | GTT | L216K | AAG | P219R | CGG |
| S210L | CTG | I213G | GGG | L216M | ATG | P219H | CAT |
| S210V | GTG | I213N | AAT | L216P | CCT | P219G | GGG |
| S210H | CAT | I213L | CTT | L216Q | CAG | P219K | AAG |
| S210N | AAT | 1213S | AGT | L216R | CGG | P219S | TCG |
| S210F | TTT | 1213M | ATG | L216S | TCT | P219W | TGG |
| S210P | CCG | 1213R | CGG | L216T | ACT | S220R | CGT |
| S210W | TGG | 1213K | AAG | L216V | GTG | S220A | GCG |
| S210Q | CAG | 1213F | TTT | L216W | TGG | S220Q | CAG |
| S210T | ACG | 1213D | GAT | M217P | CCT | S220T | ACT |
| S210K | AAG | 1213E | GAG | M217Y | TAT | S220L | CTT |
| S210A | GCG | G214L | TTG | M217T | ACG | S220K | AAG |
| T211P | CCG | G214Q | CAG | M217C | TGT | S220G | GGG |
| T211R | CGT | G214S | TCT | M217S | AGT | S220H | CAT |
| T211K | AAG | G214T | ACT | M217L | CTG | S220E | GAG |
| T211G | GGG | G214V | GTG | M217N | AAT | S220M | ATG |
| T211M | ATG | G214I | ATT | M217R | CGG | S220V | GTT |
| T211N | AAT | G214R | CGT | M217Q | CAG | S220P | CCG |
| T211V | GTG | G214P | CCG | M217K | AAG | S220I | ATT |
| T211H | CAT | G214E | GAG | M217G | GGG | S220F | TTT |
| S229N | AAT | S224T | ACG | V227K | AAG | A230S | TCG |
| Y221W | TGG | S224Q | CAG | V227L | CTG | A230C | TGT |
| Y221K | AAG | S224R | CGG | V227P | CCT | A230V | GTT |
| Y221Q | CAG | S224P | CCG | V227S | TCT | A230T | ACT |
| Y221C | TGT | S224I | ATT | V227T | ACT | A230Y | TAT |
| Y221N | AAT | S224V | GTT | V227W | TGG | A230M | ATG |
| Y221P | CCT | S224L | TTG | V227Y | TAT | A230N | AAT |
| Y221V | GTT | S224C | TGT | V227G | GGG | A230H | CAT |
| Y221A | GCG | S224K | AAG | V227H | CAT | Q231I | ATT |
| Y221G | GGG | S224D | GAT | V227Q | CAG | Q231A | GCT |
| Y221R | CGG | S224H | CAT | V227R | CGT | Q231F | TTT |
| Y221S | TCG | S224M | ATG | Q228A | GCT | Q231P | CCT |
| Y221M | ATG | S224A | GCT | Q228D | GAT | Q231Y | TAT |
| Y221T | ACG | S224W | TGG | Q228E | GAG | Q231R | CGT |
| Y221L | CTT | G225D | GAT | Q228G | GGT | Q231L | CTG |
| Y221E | GAG | G225R | CGT | Q228H | CAT | Q231D | GAT |
| T222L | TTG | G225Q | CAG | Q228K | AAG | Q231G | GGT |
| T222Y | TAT | G225M | ATG | Q228L | CTG | Q231V | GTT |
| T222R | CGT | G225P | CCT | Q228M | ATG | Q231W | TGG |
| T222V | GTT | G225W | TGG | Q228N | AAT | Q231S | AGT |
| T222P | CCT | G225S | TCT | Q228P | CCG | Q231H | CAT |
| T222S | AGT | G225E | GAG | Q228R | CGG | Q231C | TGT |
| T222A | GCT | G225V | GTT | Q228S | TCT | Q231M | ATG |
| T222H | CAT | G225T | ACG | Q228T | ACG | D232H | CAT |
| T222G | GGG | G225K | AAG | Q228W | TGG | D232G | GGG |
| T222M | ATG | G225N | AAT | Q228Y | TAT | D232R | CGT |
| T222F | TTT | G225C | TGT | L229R | CGG | D232P | CCT |
| T222C | TGT | G225H | CAT | L229A | GCG | D232Y | TAT |
| T222I | ATT | G225A | GCG | L229T | ACG | D232N | AAT |
| T222N | AAT | D226S | TCT | L229Q | CAG | D232S | TCG |
| T222W | TGG | D226W | TGG | L229P | CCT | D232F | TTT |
| T222D | GAT | D226R | CGG | L229E | GAG | D232V | GTG |
| F223L | TTG | D226A | GCT | L229W | TGG | D232K | AAG |
| F223T | ACG | D226N | AAT | L229M | ATG | D232W | TGG |
| F223C | TGT | D226T | ACT | L229I | ATT | D232Q | CAG |
| F223R | CGT | D226E | GAG | L229G | GGT | D232E | GAG |
| F223N | AAT | D226L | CTT | L229C | TGT | D232T | ACT |
| F223P | CCT | D226P | CCT | L229Y | TAT | D232L | CTG |
| F223E | GAG | D226H | CAT | L229D | GAT | D233Q | CAG |
| F223G | GGG | D226G | GGT | L229H | CAT | D233P | CCG |
| F223Q | CAG | D226I | ATT | L229V | GTG | D233S | TCT |
| F223A | GCG | D226M | ATG | A230L | TTG | D233T | ACG |
| F223S | TCT | D226V | GTG | A230G | GGT | D233A | GCG |
| F223V | TAT | D226C | TGT | A230W | TGG | D233W | TGG |
| F223H | CAT | V227A | GCT | A230P | CCG | D233G | GGT |
| F223K | AAG | V227C | TGT | A230D | GAT | D233R | CGT |
| F223M | ATG | V227D | GAT | A230R | CGT | D233E | GAG |
| S224G | GGG | V227E | GAG | A230I | ATT | D233N | AAT |
| D233V | GTG | G236N | AAT | I240G | GGG | R243L | CTT |
| D233M | ATG | G236F | TTT | I240Q | CAG | R243A | GCG |
| D233L | CTG | I237S | TCG | I240P | CCG | R243H | CAT |
| D233K | AAG | I237L | CTG | I240R | CGG | R243Q | CAG |
| D233I | ATT | I237R | CGT | I240S | TCG | R243S | AGT |
| I234A | GCT | I237Q | CAG | I240K | AAG | R243I | ATT |
| I234T | ACG | I237K | AAG | I240V | GTG | R243C | TGT |
| I234V | GTT | I237D | GAT | I240D | GAT | R243N | AAT |
| I234W | TGG | I237A | GCG | I240A | GCG | R243Y | TAT |
| I234E | GAG | I237T | ACG | I240C | TGT | R243G | GGG |
| I234G | GGT | I237E | GAG | I240L | CTT | R243D | GAT |
| I234L | CTT | I237C | TGT | I240F | TTT | R243V | GTG |
| I234H | CAT | I237G | GGG | I240Y | TAT | S244P | CCG |
| I234M | ATG | I237P | CCT | I240M | ATG | S244L | CTT |
| I234N | AAT | I237Y | TAT | I240T | ACG | S244W | TGG |
| I234Y | TAT | I237W | TGG | Y241V | GTT | S244M | ATG |
| I234P | CCT | I237N | AAT | Y241A | GCT | S244V | GTT |
| I234D | GAT | Q238G | GGG | Y241G | GGG | S244Q | CAG |
| I234Q | CAG | Q238H | CAT | Y241H | CAT | S244D | GAT |
| I234C | TGT | Q238S | TCG | Y241R | CGG | S244E | GAG |
| D235H | CAT | Q238Y | TAT | Y241P | CCG | S244T | ACG |
| D235G | GGG | Q238F | TTT | Y241Q | CAG | S244H | CAT |
| D235A | GCG | Q238E | GAG | Y241L | TTG | S244G | GGT |
| D235P | CCG | Q238L | TTG | Y241T | ACG | S244A | GCT |
| D235L | CTT | Q238W | TGG | Y241S | AGT | S244F | TTT |
| D235V | GTG | Q238P | CCG | Y241W | TGG | S244Y | TAT |
| D235E | GAG | Q238R | AGG | Y241N | AAT | S244R | CGT |
| D235R | CGT | Q238C | TGT | Y241M | ATG | Q245P | CCT |
| D235Q | CAG | Q238N | AAT | Y241I | ATT | Q245I | ATT |
| D235T | ACG | Q238I | ATT | Y241D | GAT | Q245F | TTT |
| D235C | TGT | Q238T | ACG | G242A | GCG | Q245V | GTT |
| D235S | TCG | Q238K | AAG | G242F | TTT | Q245M | ATG |
| D235N | AAT | A239S | TCT | G242L | CTT | Q245T | ACT |
| D235Y | TAT | A239Q | CAG | G242N | AAT | Q245E | GAG |
| D235I | ATT | A239T | ACG | G242P | CCT | Q245S | TCG |
| G236M | ATG | A239P | CCT | G242W | TGG | Q245R | CGG |
| G236R | CGG | A239V | GTG | G242T | ACG | Q245G | GGT |
| G236D | GAT | A239L | CTG | G242R | CGT | Q245H | CAT |
| G236S | TCT | A239Y | TAT | G242V | GTT | Q245L | CTT |
| G236T | ACT | A239I | ATT | G242S | TCG | Q245K | AAG |
| G236C | TGT | A239C | TGT | G242I | ATT | Q245W | TGG |
| G236K | AAG | A239G | GGG | G242Y | TAT | Q245C | TGT |
| G236E | GAG | A239W | TGG | G242H | CAT | N246W | TGG |
| G236P | CCG | A239F | TTT | G242E | GAG | N246R | CGG |
| G236I | ATT | A239K | AAG | G242K | AAG | N246A | GCG |
| G236Y | TAT | A239H | CAT | R243P | CCG | N246F | TTT |
| G236L | CTG | A239R | CGT | R243K | AAG | N246G | GGT |
| G236V | GTT | A239D | GAT | R243T | ACG | N246P | CCT |
| N246V | GTT | Q249G | GGT | G252P | CCT | T255L | TTG |
| N246Q | CAG | Q249N | AAT | G252H | CAT | T255H | CAT |
| N246Y | TAT | Q249K | AAG | G252C | TGT | P256S | AGT |
| N246C | TGT | Q249I | ATT | G252V | GTT | P256V | GTG |
| N246I | ATT | Q249Y | TAT | G252I | ATT | P256F | TTT |
| N246L | TTG | Q249V | GTG | P253C | TGT | P256Y | TAT |
| N246S | TCT | Q249L | TTG | P253G | GGT | P256I | ATT |
| N246T | ACT | Q249H | CAT | P253Q | CAG | P256A | GCT |
| N246K | AAG | P250L | CTG | P253I | ATT | P256L | CTT |
| N246D | GAT | P250S | TCG | P253L | CTG | P256G | GGT |
| P247A | GCG | P250R | CGG | P253R | CGG | P256N | AAT |
| P247D | GAT | P250Y | TAT | P253A | GCT | P256R | CGG |
| P247E | GAG | P250M | ATG | P253E | GAG | P256Q | CAG |
| P247F | TTT | P250F | TTT | P253Y | TAT | P256E | GAG |
| P247G | GGG | P250A | GCT | P253W | TGG | P256K | AAG |
| P247H | CAT | P250K | AAG | P253M | ATG | P256M | ATG |
| P247I | ATT | P250G | GGT | P253V | GTG | P256C | TGT |
| P247K | AAG | P250N | AAT | P253T | ACT | K257C | TGT |
| P247L | CTG | P250T | ACT | P253K | AAG | K257M | ATG |
| P247N | AAT | P250W | TGG | P253N | AAT | K257V | GTT |
| P247Q | CAG | P250D | GAT | Q254R | CGT | K257A | GCT |
| P247R | CGT | P250V | GTG | Q254G | GGG | K257E | GAG |
| P247S | TCG | P250Q | CAG | Q254W | TGG | K257S | TCT |
| P247T | ACG | I251A | GCG | Q254T | ACT | K257L | CTT |
| P247V | GTT | I251Q | CAG | Q254A | GCT | K257I | ATT |
| V248W | TGG | I251G | GGG | Q254F | TTT | K257G | GGG |
| V248L | CTG | I251L | CTG | Q254D | GAT | K257N | AAT |
| V248Q | CAG | I251K | AAG | Q254P | CCG | K257F | TTT |
| V248M | ATG | I251R | CGT | Q254L | CTG | K257W | TGG |
| V248Y | TAT | I251E | GAG | Q254C | TGT | K257R | CGG |
| V248G | GGG | I251D | GAT | Q254Y | TAT | K257P | CCG |
| V248C | TGT | I251T | ACG | Q254I | ATT | K257T | ACT |
| V248R | CGG | I251C | TGT | Q254E | GAG | A258Q | CAG |
| V248A | GCG | I251Y | TAT | Q254V | GTG | A258Y | TAT |
| V248H | CAT | I251P | CCT | Q254S | TCT | A258W | TGG |
| V248I | ATT | I251S | TCT | T255I | ATT | A258G | GGG |
| V248T | ACT | 1251W | TGG | T255Q | CAG | A258L | TTG |
| V248K | AAG | 1251V | GTT | T255P | CCG | A258F | TTT |
| V248S | TCG | G252F | TTT | T255R | CGT | A258M | ATG |
| V248F | TTT | G252W | TGG | T255C | TGT | A258N | AAT |
| V248E | GAG | G252A | GCG | T255N | AAT | A258V | GTG |
| Q249T | ACT | G252R | CGG | T255S | AGT | A258T | ACG |
| Q249W | TGG | G252L | CTT | T255V | GTG | A258I | ATT |
| Q249R | CGG | G252E | GAG | T255E | GAG | A258D | GAT |
| Q249E | GAG | G252D | GAT | T255G | GGG | A258R | CGT |
| Q249A | GCT | G252K | AAG | T255K | AAG | A258E | GAG |
| Q249P | CCG | G252S | TCG | T255A | GCT | A258P | CCG |
| Q249C | TGT | G252T | ACG | T255F | TTT | T255L | TTG |

1. Expression

The DNA encoding each individual library member was generated according to standard DNA synthesis protocols and protein was expressed using routine molecular biology techniques. Briefly, the DNA was ligated into vector pET303CTHis (Invitrogen, SEQ ID NO:3466) using routine molecular biology techniques. Plasmid containing one individual hMMP-1 Imutant was transformed into BL21 (DE3) E. coli cells (Tigen, Beiging, China) using manufacturers recommendations. The process was repeated for all library members. The transformation culture was used to inoculate 1 mL LB medium containing ampicillin additives. The culture was grown at 37° C. with shaking for 16 hours. Protein expression was induced by the addition of 1 mM isopropyl-β-D-thiogalactaside (IPTG) and the culture was incubated at 25° C. with shaking. After 6 hours, the cells were pelleted by centrifugation at 6,000 g for 10 minutes and the supernatant was removed. The periplasmic protein was enriched by incubating the cells in 50 μl OS buffer (200 mM Tris-HCl, pH 7.5, 20% sucrose, 1 mM EDTA) with 4 μl DNAse (10 μg/ml), 4 μl RNAse (10 μg/ml), and 4 μl lysozyme (10 μg/ml) for 10 minutes at 25° C. 50 μl of water was added to each well followed by centrifugation at 6000 g for 10 minutes to remove cell debris. The supernatant, containing the hMMP-1 protein, was stored at −20° C. Activity of supernatants were screened as described in the following examples.

B. Cloning and Expression of Wildtype hMMP-1

In this example, wildtype hMMP-1 was individually expressed in both E. coli and CHO-S cells.

1. Expression in E. coli

Wildtype hMMP-1 (clone BAP006_{—}10, having a sequence of nucleotides set forth as nucleotides in SEQ ID NO:706 and containing a pel B signal sequence set forth in SEQ ID NO:3547) was cloned into vector pET303CTHis (Invitrogen, SEQ ID NO:3466) and grown in BL21(DE3) E. coli. The pET303CTHis vector contained a C-terminal His tag (SEQ ID NO:3465). Protein expression was induced upon the addition of 1 mM isopropyl-β-D-thiogalactoside (IPTG) as described above. Following expression, the protein was enriched as described in Example 1A, and subsequently purified using a HiTrap Ni²⁺ column (GE Healthcare) according to standard molecular biology protocols. Expression and purification were monitored by SDS/PAGE and Western blot analysis.

2. Expression in CHO-S Cells

Wildtype hMMP-1 (clone BAPOO6_{—}2, having a sequence of nucleotides set forth as nucleotides 72-1478 in SEQ ID NO:708 and a sequence encoding a C-terminal His tag) was expressed in CHO-S cells and secreted into the medium. Transfected cells were cultured at 37° C. in CD-CHO serum free media (Invitrogen). The wildtype hMMP-1 protein was purified using a HiTrap Ni²⁺ column (GE Healthcare) according to standard molecular biology protocols

### Example 2

### Determination of Enzymatic Activity of the hMMP-1 Mutants Using a Fluorogenic Peptide Substrate

In this example, the hMMP-1 mutant library, generated in Example 1, was screened using a high throughput fluorescence activity assay to identify temperature sensitive hMMP-1 mutants. To screen for temporally sensitive hMMP-1 mutants, the enzymatic activity of each individual mutant was determined at 25° C. and 37° C. and/or 34° C., using a commercially available fluorogenic substrate, peptide IX, designated as Mca-K-P-L-G-L-Dpa-A-R-NH₂ (SEQ ID NO:707; Mca=(7-Methoxycoumarin-4-yl)acetyl; Dpa=N-3-(2,4,-Dinitrophenyl)-L-2,3-diaminopropionyl; R&D Systems, Minneapolis, Minn., Cat# ES010). The peptide substrate contains a highly fluorescent 7-methoxycoumarin group that is quenched by resonance energy transfer to the 2,4-dinitrophenyl group. Activated hMMP-1 cleaves the amide bond between glycine and leucine resulting in an increase in released fluorescence. Reactions were initially performed in a 96-well assay and confirmed using a 14 ml tube format.

A. 96-Well Assay

Prior to assessing activity of the supernatants, supernatants were treated with a processing agent to activate the inactive zymogen form into an active enzyme. Briefly, 4 μl of each hMMP-1 mutant supernatant generated in Example 1 was added to 100 μl of TCNB (50 nM Tris, 10 mM CaCl₂, 150 mM NaCl, 0.05% Brij 35, pH 7.5) with 1 mM of the processing agent p-aminophenylmercuric acetate (APMA) in a 96-well plate. The solution was incubated at the reaction temperature (either 25° C. or 37° C.) for 2 hours. This activation step cleaves the pro-peptide and generates mature hMMP-1.

Following activation, 1.6 μl of TCNB containing 620 μM Mca-K-P-L-G-L-Dpa-A-R-NH₂ fluorescent substrate was added to each well to a final concentration of 10 μM, at the indicated reaction temperature (either 25° C. or 37° C.) for 1 hour. Fluorescence was detected by measuring fluorescence in a fluorescent plate reader at 320 nm exitation/405 nm emission. Relative fluorescence units (RFU) were determined. Supernatant from wildtype hMMP-1 and plasmid/vector transformed cells were used as positive and negative controls. Duplicate reactions were performed for each sample, reaction temperature, and positive and negative control.

The results of the initial screen of 2687 hMMP-1 mutants are shown in Table 9. The initial screen resulted in the identification of 199 putative primary hits (see Table 10) with reduced activity at 37° C. as compared to the activity at 25° C.

TABLE 9
Results of Initial Screen for Temperature Sensitive Mutants

| | | | | | | Res. | Res. |
|---|---|---|---|---|---|---|---|
| | | | | | Ratio | Act. | Act. |
| Temp. | hMMP-1 | SEQ ID | Avg. RFU | Avg. RFU | 25° C./ | Mut/wt | Mut/wt |
| Phenotype | mutation | NO | 25° C. | 37° C. | 37° C. | 25° C. | 37° C. |
| Down | F81C | 784 | 1740.62 | 3123.63 | 0.56 | 0.35 | 0.46 |
| Down | F81E | 780 | 871.51 | 1243.66 | 0.70 | 0.18 | 0.18 |
| Down | F811 | 793 | 4100.22 | 5376.62 | 0.76 | 0.83 | 0.79 |
| Neutral | F81L | 795 | 8890.68 | 7913.44 | 1.12 | 1.57 | 1.51 |
| Neutral | F81P | 797 | 1102.23 | 1043.87 | 1.06 | 0.19 | 0.20 |
| Neutral | F81S | 789 | 2527.30 | 2312.47 | 1.09 | 0.45 | 0.44 |
| Neutral | F81A | 796 | 8780.53 | 7784.51 | 1.13 | 1.55 | 1.48 |
| Neutral | F81M | 791 | 2545.25 | 3095.21 | 0.82 | 0.45 | 0.59 |
| Neutral | F81G | 790 | 8979.05 | 7773.71 | 1.16 | 1.59 | 1.48 |
| Neutral | F81T | 787 | 1564.49 | 1373.60 | 1.14 | 0.28 | 0.26 |
| Neutral | F81Q | 786 | 9225.28 | 7923.69 | 1.16 | 1.63 | 1.51 |
| Neutral | F81R | 783 | 8514.40 | 7454.74 | 1.14 | 1.50 | 1.42 |
| Neutral | F81W | 792 | 6078.70 | 5909.04 | 1.03 | 1.07 | 1.12 |
| Neutral | F81H | 781 | 8126.15 | 7360.21 | 1.10 | 1.44 | 1.40 |
| Neutral | F81V | 794 | 7263.15 | 6614.17 | 1.10 | 1.28 | 1.26 |
| Neutral | V821 | 813 | 535.78 | 548.02 | 0.98 | 0.06 | 0.06 |
| Down | V82C | 803 | 4177.57 | 6476.29 | 0.65 | 0.50 | 0.72 |
| Neutral | V82A | 815 | 9540.61 | 9240.92 | 1.03 | 1.14 | 1.03 |
| Neutral | V82P | 816 | 599.23 | 634.69 | 0.94 | 0.07 | 0.07 |
| Down | V82Y | 807 | 3295.59 | 6173.45 | 0.53 | 0.39 | 0.69 |
| Down | V82M | 811 | 6824.39 | 8606.64 | 0.79 | 0.82 | 0.96 |
| Neutral | V82Q | 805 | 581.51 | 652.74 | 0.89 | 0.07 | 0.07 |
| Neutral | V82F | 810 | 7233.54 | 8739.45 | 0.83 | 0.87 | 0.98 |
| Down | V82W | 812 | 6194.12 | 8397.19 | 0.74 | 0.74 | 0.94 |
| Neutral | V82N | 804 | 9421.72 | 8759.51 | 1.08 | 1.13 | 0.98 |
| Down | V82R | 802 | 603.22 | 781.77 | 0.77 | 0.07 | 0.09 |
| Neutral | V82G | 809 | 8298.42 | 8911.04 | 0.93 | 0.99 | 0.99 |
| Neutral | V82S | 808 | 8293.03 | 9022.13 | 0.92 | 0.99 | 1.01 |
| Down | V82L | 814 | 6951.75 | 8694.05 | 0.80 | 0.83 | 0.97 |
| Neutral | V82T | 806 | 7993.81 | 8975.05 | 0.89 | 0.96 | 1.00 |
| Neutral | L83A | 834 | 8629.03 | 9023.51 | 0.96 | 1.03 | 1.01 |
| Neutral | L83C | 822 | 554.26 | 567.87 | 0.98 | 0.07 | 0.06 |
| Neutral | L83D | 817 | 8705.34 | 8957.38 | 0.97 | 1.04 | 1.00 |
| Neutral | L83E | 818 | 9212.48 | 9265.02 | 0.99 | 1.10 | 1.03 |
| Neutral | L83G | 828 | 7713.92 | 9073.74 | 0.85 | 0.92 | 1.01 |
| Neutral | L83H | 819 | 6449.24 | 7800.76 | 0.83 | 0.77 | 0.87 |
| Down | L831 | 832 | 4575.76 | 6963.24 | 0.66 | 0.55 | 0.78 |
| Down | L83M | 830 | 5921.65 | 8064.61 | 0.73 | 0.71 | 0.90 |
| Neutral | L83P | 835 | 7794.15 | 8608.36 | 0.91 | 0.93 | 0.96 |
| Neutral | L83Q | 824 | 7291.24 | 8673.39 | 0.84 | 0.87 | 0.97 |
| Neutral | L83R | 821 | 8509.58 | 8988.62 | 0.95 | 1.02 | 1.00 |
| Neutral | L83S | 827 | 9261.79 | 9205.93 | 1.01 | 1.11 | 1.03 |
| Neutral | L83T | 825 | 7549.73 | 8580.54 | 0.88 | 0.90 | 0.96 |
| Down | L83W | 831 | 4193.18 | 6044.52 | 0.69 | 0.50 | 0.67 |
| Neutral | L83Y | 826 | 7968.79 | 9051.39 | 0.88 | 0.95 | 1.01 |
| Down | T84V | 851 | 3169.35 | 4931.29 | 0.64 | 0.64 | 0.72 |
| Down | T84E | 837 | 498.18 | 627.84 | 0.79 | 0.10 | 0.09 |
| Neutral | T84H | 838 | 7046.83 | 6974.20 | 1.01 | 1.24 | 1.33 |
| Neutral | T84L | 852 | 7687.84 | 6946.59 | 1.11 | 1.36 | 1.32 |
| Neutral | T84D | 836 | 7972.32 | 7331.43 | 1.09 | 1.41 | 1.39 |
| Neutral | T84R | 840 | 7298.49 | 6880.17 | 1.06 | 1.29 | 1.31 |
| Neutral | T841 | 850 | 6508.69 | 5860.75 | 1.11 | 1.15 | 1.11 |
| Neutral | T84S | 845 | 6073.28 | 5981.85 | 1.02 | 1.07 | 1.14 |
| Neutral | T84G | 846 | 8087.79 | 7200.99 | 1.12 | 1.43 | 1.37 |
| Neutral | T84Q | 843 | 6275.12 | 6690.38 | 0.94 | 1.11 | 1.27 |
| Neutral | T84P | 854 | 3528.37 | 3832.34 | 0.92 | 0.62 | 0.73 |
| Neutral | T84A | 853 | 8718.27 | 7840.72 | 1.11 | 1.54 | 1.49 |
| Neutral | T84C | 841 | 5177.89 | 5107.57 | 1.01 | 0.91 | 0.97 |
| Neutral | T84Y | 844 | 4768.51 | 4818.30 | 0.99 | 0.84 | 0.92 |
| Neutral | T84F | 847 | 6312.72 | 6453.46 | 0.98 | 1.10 | 1.27 |
| Down | E85L | 871 | 1633.29 | 2148.43 | 0.76 | 0.33 | 0.31 |
| Down | E85Q | 861 | 2834.50 | 4068.60 | 0.70 | 0.57 | 0.59 |
| Neutral | E85P | 873 | 2855.52 | 3389.51 | 0.84 | 0.58 | 0.50 |
| Neutral | E85T | 862 | 401.26 | 382.58 | 1.05 | 0.08 | 0.06 |
| Down | E85K | 857 | 2293.84 | 3049.87 | 0.75 | 0.46 | 0.45 |
| Down | E85M | 867 | 2158.30 | 2821.39 | 0.76 | 0.44 | 0.41 |
| Neutral | E85G | 865 | 1767.69 | 1734.31 | 1.02 | 0.31 | 0.33 |
| Down | E85R | 858 | 912.46 | 7286.41 | 0.13 | 0.16 | 1.39 |
| Neutral | E85S | 864 | 7811.54 | 7488.09 | 1.04 | 1.38 | 1.42 |
| Neutral | E85C | 859 | 6027.10 | 5938.05 | 1.01 | 1.06 | 1.13 |
| Neutral | E85Y | 863 | 4449.33 | 3909.71 | 1.14 | 0.79 | 0.74 |
| Neutral | E85A | 872 | 5552.19 | 5461.08 | 1.02 | 0.98 | 1.04 |
| Down | E85N | 860 | 522.81 | 7634.45 | 0.07 | 0.09 | 1.45 |
| Neutral | E85V | 870 | 7152.74 | 7011.60 | 1.02 | 1.26 | 1.33 |
| Neutral | E85F | 866 | 6092.47 | 6362.37 | 0.96 | 1.06 | 1.26 |
| Down | G86L | 890 | 2452.10 | 3232.22 | 0.76 | 0.50 | 0.47 |
| Down | G86P | 892 | 2117.46 | 5219.90 | 0.41 | 0.43 | 0.76 |
| Neutral | G861 | 888 | 1888.26 | 2293.71 | 0.82 | 0.38 | 0.34 |
| Neutral | G86T | 882 | 363.85 | 380.61 | 0.96 | 0.07 | 0.06 |
| Neutral | G86H | 876 | 389.15 | 372.78 | 1.04 | 0.08 | 0.05 |
| Neutral | G86D | 874 | 415.45 | 406.81 | 1.02 | 0.08 | 0.06 |
| Down | G86N | 880 | 2612.85 | 3755.02 | 0.70 | 0.53 | 0.55 |
| Neutral | G86S | 884 | 8500.13 | 7717.19 | 1.10 | 1.50 | 1.47 |
| Neutral | G86K | 877 | 1660.95 | 2002.39 | 0.83 | 0.29 | 0.38 |
| Neutral | G86W | 887 | 1570.85 | 1690.05 | 0.93 | 0.28 | 0.32 |
| Neutral | G86Y | 883 | 1829.24 | 2126.68 | 0.86 | 0.32 | 0.40 |
| Neutral | G86V | 889 | 1830.80 | 2092.69 | 0.87 | 0.32 | 0.40 |
| Neutral | G86C | 879 | 1784.05 | 2091.03 | 0.85 | 0.32 | 0.40 |
| Neutral | G86M | 886 | 1687.28 | 2025.99 | 0.83 | 0.30 | 0.39 |
| Up | G86F | 885 | 1897.87 | 1483.82 | 1.28 | 0.34 | 0.28 |
| Neutral | N87M | 905 | 418.35 | 412.23 | 1.01 | 0.08 | 0.06 |
| Down | N87L | 909 | 3385.42 | 4941.20 | 0.69 | 0.69 | 0.72 |
| Neutral | N87P | 911 | 8762.48 | 8941.20 | 0.98 | 1.55 | 1.70 |
| Neutral | N87V | 908 | 6199.21 | 7269.38 | 0.85 | 1.09 | 1.38 |
| Neutral | N87R | 897 | 7761.00 | 8810.25 | 0.88 | 1.37 | 1.68 |
| Up | N87F | 904 | 6882.19 | 4428.08 | 1.55 | 1.22 | 0.84 |
| Down | N87S | 902 | 2083.05 | 3304.46 | 0.63 | 0.37 | 0.63 |
| Neutral | N871 | 907 | 7572.66 | 8090.13 | 0.94 | 1.34 | 1.54 |
| Neutral | N87C | 898 | 3291.22 | 3945.40 | 0.83 | 0.58 | 0.75 |
| Down | N87A | 910 | 5482.33 | 6869.11 | 0.80 | 0.97 | 1.31 |
| Neutral | N87G | 903 | 8060.01 | 8916.11 | 0.90 | 1.42 | 1.70 |
| Down | N87Y | 901 | 4397.56 | 5611.87 | 0.78 | 0.78 | 1.07 |
| Up | N87E | 894 | 5876.33 | 4763.86 | 1.23 | 1.04 | 0.91 |
| Down | N87H | 895 | 5013.05 | 7306.33 | 0.69 | 0.89 | 1.39 |
| Neutral | N87Q | 899 | 8559.37 | 9021.72 | 0.95 | 1.51 | 1.72 |
| Down | P88C | 917 | 1255.12 | 2197.65 | 0.57 | 0.15 | 0.25 |
| Neutral | P88K | 915 | 6857.61 | 8492.90 | 0.81 | 0.82 | 0.95 |
| Down | P88W | 926 | 664.95 | 845.70 | 0.79 | 0.08 | 0.09 |
| Down | P88G | 923 | 1694.96 | 3159.20 | 0.54 | 0.20 | 0.35 |
| Down | P88L | 929 | 2562.59 | 3576.95 | 0.72 | 0.31 | 0.40 |
| Down | P88Q | 919 | 4499.52 | 7270.91 | 0.62 | 0.54 | 0.81 |
| Neutral | P88A | 930 | 6549.92 | 8130.83 | 0.81 | 0.78 | 0.91 |
| Neutral | P88T | 920 | 6576.99 | 8126.45 | 0.81 | 0.79 | 0.91 |
| Down | P88Y | 921 | 5515.19 | 7868.29 | 0.70 | 0.66 | 0.88 |
| Down | P88R | 916 | 4209.25 | 6681.38 | 0.63 | 0.50 | 0.75 |
| Down | P88H | 914 | 2580.97 | 4465.31 | 0.58 | 0.31 | 0.50 |
| Down | P881 | 927 | 841.81 | 1249.17 | 0.67 | 0.10 | 0.14 |
| Neutral | P88V | 928 | 1666.69 | 1915.49 | 0.87 | 0.20 | 0.21 |
| Down | P88E | 913 | 971.61 | 1460.63 | 0.67 | 0.12 | 0.16 |
| Down | P88D | 912 | 1300.22 | 1911.83 | 0.68 | 0.16 | 0.21 |
| Down | R89V | 946 | 163.86 | 2620.05 | 0.44 | 0.24 | 0.38 |
| Down | R89W | 944 | 1252.89 | 1744.18 | 0.72 | 0.25 | 0.25 |
| Neutral | R89M | 943 | 402.00 | 386.98 | 1.04 | 0.08 | 0.06 |
| Neutral | R89A | 948 | 7883.15 | 8954.83 | 0.88 | 1.39 | 1.70 |
| Neutral | R89T | 938 | 6791.27 | 6752.46 | 1.01 | 1.20 | 1.28 |
| Neutral | R89G | 941 | 8957.06 | 8693.72 | 1.03 | 1.58 | 1.65 |
| Up | R89S | 940 | 7342.24 | 4138.54 | 1.77 | 1.30 | 0.79 |
| Neutral | R89K | 934 | 7679.02 | 8254.00 | 0.93 | 1.36 | 1.57 |
| Neutral | R89F | 942 | 4764.35 | 5589.97 | 0.85 | 0.84 | 1.06 |
| Neutral | R89Y | 939 | 5614.23 | 5949.31 | 0.94 | 0.99 | 1.13 |
| Up | R89N | 936 | 3502.08 | 1995.86 | 1.75 | 0.62 | 0.38 |
| Neutral | R89H | 933 | 3611.69 | 4222.47 | 0.86 | 0.64 | 0.80 |
| Neutral | R89L | 947 | 3123.66 | 3332.30 | 0.94 | 0.55 | 0.63 |
| Neutral | R89E | 932 | 1490.93 | 1265.89 | 1.18 | 0.26 | 0.24 |
| Down | R89P | 949 | 2659.02 | 3342.56 | 0.80 | 0.47 | 0.64 |
| Neutral | W90L | 966 | 394.24 | 411.82 | 0.96 | 0.08 | 0.06 |
| Neutral | W90G | 961 | 448.08 | 427.28 | 1.05 | 0.09 | 0.06 |
| Neutral | W90P | 968 | 444.72 | 442.65 | 1.00 | 0.09 | 0.06 |
| Neutral | W90T | 958 | 397.42 | 365.04 | 1.09 | 0.08 | 0.05 |
| Neutral | W90S | 960 | 443.43 | 442.72 | 1.00 | 0.09 | 0.06 |
| Neutral | W90V | 965 | 384.57 | 385.18 | 1.00 | 0.08 | 0.06 |
| Neutral | W901 | 964 | 443.81 | 432.28 | 1.03 | 0.09 | 0.06 |
| Neutral | W90A | 967 | 497.94 | 554.07 | 0.90 | 0.10 | 0.08 |
| Neutral | W90F | 962 | 730.98 | 656.84 | 1.11 | 0.15 | 0.10 |
| Neutral | W90H | 952 | 498.51 | 493.15 | 1.01 | 0.10 | 0.07 |
| Neutral | W90M | 963 | 512.18 | 508.03 | 1.01 | 0.10 | 0.07 |
| Neutral | W90R | 954 | 1974.98 | 1695.11 | 1.17 | 0.23 | 0.19 |
| Up | W90E | 951 | 1537.84 | 1076.32 | 1.43 | 0.18 | 0.12 |
| Up | W90N | 956 | 1308.11 | 1001.91 | 1.31 | 0.15 | 0.11 |
| U p | W90Q | 957 | 1392.58 | 1015.03 | 1.37 | 0.16 | 0.12 |
| Down | E91N | 974 | 4746.43 | 6166.37 | 0.77 | 0.96 | 0.90 |
| Down | E91R | 972 | 2760.48 | 3810.12 | 0.72 | 0.56 | 0.56 |
| Down | E91W | 982 | 2595.35 | 5651.48 | 0.46 | 0.53 | 0.83 |
| Down | E91G | 979 | 4826.02 | 6684.79 | 0.72 | 0.98 | 0.98 |
| Neutral | E91V | 984 | 454.87 | 459.17 | 0.99 | 0.09 | 0.07 |
| Neutral | E91Y | 977 | 4885.18 | 5469.16 | 0.89 | 0.99 | 0.80 |
| Down | E91C | 973 | 3525.68 | 5567.75 | 0.63 | 0.71 | 0.81 |
| Down | E91H | 970 | 5114.86 | 6610.88 | 0.77 | 1.04 | 0.97 |
| Neutral | E91T | 976 | 442.21 | 427.42 | 1.03 | 0.09 | 0.06 |
| Neutral | E91S | 978 | 8147.93 | 7696.77 | 1.06 | 0.94 | 0.87 |
| Neutral | E91A | 986 | 1140.60 | 1252.34 | 0.91 | 0.13 | 0.14 |
| Neutral | E911 | 983 | 8414.79 | 8744.30 | 0.96 | 0.97 | 0.99 |
| Neutral | E91D | 969 | 8482.61 | 8681.73 | 0.98 | 0.98 | 0.98 |
| Neutral | E91F | 980 | 1159.80 | 1117.15 | 1.04 | 0.13 | 0.13 |
| Neutral | E91L | 985 | 2012.22 | 1956.07 | 1.03 | 0.23 | 0.22 |
| Down | Q92V | 1003 | 3748.94 | 5787.25 | 0.65 | 0.76 | 0.85 |
| Down | Q92Y | 996 | 2141.40 | 5383.55 | 0.40 | 0.43 | 0.79 |
| Down | Q92L | 1004 | 2422.01 | 3765.30 | 0.64 | 0.49 | 0.55 |
| Neutral | Q92N | 994 | 8685.91 | 8183.03 | 1.06 | 1.00 | 0.93 |
| Neutral | Q92E | 989 | 8489.89 | 8972.33 | 0.95 | 0.98 | 1.02 |
| Neutral | Q921 | 1002 | 7791.35 | 8518.64 | 0.91 | 0.90 | 0.97 |
| Neutral | Q92T | 995 | 8289.96 | 8916.74 | 0.93 | 0.96 | 1.01 |
| Neutral | Q92G | 998 | 7218.56 | 8372.74 | 0.86 | 0.83 | 0.95 |
| Neutral | Q92P | 1006 | 3678.59 | 4021.57 | 0.91 | 0.43 | 0.46 |
| Neutral | Q92W | 1001 | 7277.76 | 8042.96 | 0.90 | 0.84 | 0.91 |
| Neutral | Q92F | 999 | 8216.00 | 8989.59 | 0.91 | 0.95 | 1.02 |
| Neutral | Q92S | 997 | 8760.81 | 9254.16 | 0.95 | 1.01 | 1.05 |
| Neutral | Q92R | 992 | 8566.65 | 8894.65 | 0.96 | 0.99 | 1.01 |
| Neutral | Q92K | 991 | 8790.93 | 9239.36 | 0.95 | 1.02 | 1.05 |
| Neutral | Q92A | 1005 | 8138.84 | 9037.58 | 0.90 | 0.94 | 1.02 |
| Down | T93A | 1024 | 2321.71 | 5447.47 | 0.43 | 0.47 | 0.80 |
| Neutral | T93L | 1023 | 541.85 | 545.89 | 0.99 | 0.11 | 0.08 |
| Down | T93M | 1019 | 5256.54 | 7088.24 | 0.74 | 1.07 | 1.04 |
| Neutral | T93N | 1013 | 5852.91 | 7141.52 | 0.82 | 1.19 | 1.04 |
| Neutral | T93V | 1022 | 7976.61 | 8668.81 | 0.92 | 0.92 | 0.98 |
| Neutral | T931 | 1021 | 9015.76 | 9426.26 | 0.96 | 1.04 | 1.07 |
| Neutral | T93D | 1007 | 8742.88 | 9032.61 | 0.97 | 1.01 | 1.02 |
| Neutral | T93S | 1016 | 8832.30 | 8978.09 | 0.98 | 1.02 | 1.02 |
| Neutral | T93R | 1011 | 8802.98 | 8782.70 | 1.00 | 1.02 | 1.00 |
| Neutral | T93W | 1020 | 7872.73 | 8474.20 | 0.93 | 0.91 | 0.96 |
| Down | T93F | 1018 | 4307.35 | 5656.46 | 0.76 | 0.50 | 0.64 |
| Neutral | T93P | 1025 | 8315.28 | 8629.67 | 0.96 | 0.96 | 0.98 |
| Down | T93G | 1017 | 4926.38 | 6453.11 | 0.76 | 0.57 | 0.73 |
| Neutral | T93K | 1010 | 8581.02 | 8663.14 | 0.99 | 0.99 | 0.98 |
| Neutral | T93E | 1008 | 8081.66 | 8373.46 | 0.97 | 0.93 | 0.95 |
| Neutral | H94L | 1042 | 509.44 | 507.83 | 1.00 | 0.10 | 0.07 |
| Down | H94S | 1035 | 3442.98 | 5184.16 | 0.66 | 0.70 | 0.76 |
| Neutral | H94M | 1038 | 7388.19 | 8302.74 | 0.89 | 0.85 | 0.94 |
| Neutral | H94R | 1029 | 7237.77 | 7718.22 | 0.94 | 0.84 | 0.87 |
| Neutral | H94E | 1027 | 8375.45 | 8466.04 | 0.99 | 0.97 | 0.96 |
| Neutral | H94I | 1040 | 6326.35 | 7655.54 | 0.83 | 0.73 | 0.87 |
| Neutral | H94D | 1026 | 7358.29 | 8057.05 | 0.91 | 0.85 | 0.91 |
| Neutral | H94P | 1044 | 2892.06 | 3183.37 | 0.91 | 0.33 | 0.36 |
| Neutral | H94A | 1043 | 8285.72 | 8772.41 | 0.94 | 0.96 | 0.99 |
| Neutral | H94N | 1031 | 8497.48 | 8732.16 | 0.97 | 0.98 | 0.99 |
| Down | H94F | 1037 | 6046.02 | 7839.76 | 0.77 | 0.70 | 0.89 |
| Neutral | H94G | 1036 | 7671.85 | 7912.46 | 0.97 | 0.89 | 0.90 |
| Neutral | H94T | 1033 | 7121.14 | 8100.48 | 0.88 | 0.82 | 0.92 |
| Neutral | H94V | 1041 | 7941.67 | 8381.81 | 0.95 | 0.92 | 0.95 |
| Neutral | H94W | 1039 | 6520.52 | 7583.08 | 0.86 | 0.75 | 0.86 |
| Down | L95E | 4 | 4165.44 | 5381.52 | 0.77 | 0.48 | 0.61 |
| Neutral | L95Y | 12 | 1044.63 | 1118.92 | 0.93 | 0.12 | 0.13 |
| Neutral | L9SR | 7 | 1328.79 | 1312.13 | 1.01 | 0.15 | 0.15 |
| Neutral | L95A | 20 | 1262.99 | 1297.06 | 0.97 | 0.15 | 0.15 |
| Neutral | L95G | 14 | 1090.24 | 1183.93 | 0.92 | 0.13 | 0.13 |
| Up | L95K | 6 | 1333.28 | 1191.46 | 1.12 | 0.15 | 0.14 |
| Neutral | L95S | 13 | 1077.02 | 1117.02 | 0.96 | 0.12 | 0.13 |
| Neutral | L95T | 11 | 1407.58 | 1310.18 | 1.07 | 0.16 | 0.15 |
| Neutral | L95H | 5 | 1270.21 | 1086.69 | 1.17 | 0.15 | 0.12 |
| Neutral | L95W | 17 | 1133.63 | 1041.65 | 1.09 | 0.13 | 0.12 |
| Neutral | L95V | 19 | 8390.57 | 8371.68 | 1.00 | 0.97 | 0.95 |
| Neutral | L95C | 8 | 2189.50 | 2519.34 | 0.87 | 0.25 | 0.29 |
| Neutral | L95P | 21 | 1084.88 | 1147.69 | 0.95 | 0.13 | 0.13 |
| Neutral | L95D | 3 | 909.41 | 933.49 | 0.97 | 0.11 | 0.11 |
| Down | L951 | 18 | 1707.98 | 2294.02 | 0.74 | 0.30 | 0.45 |
| Neutral | T96E | 1046 | 415.05 | 397.38 | 1.04 | 0.07 | 0.06 |
| Neutral | T96R | 1049 | 478.81 | 441.86 | 1.08 | 0.08 | 0.07 |
| Neutral | T96P | 1063 | 589.64 | 692.90 | 0.85 | 0.09 | 0.11 |
| Down | T96S | 1054 | 3055.53 | 4011.47 | 0.76 | 0.49 | 0.64 |
| Neutral | T96A | 1062 | 1873.45 | 2254.28 | 0.83 | 0.30 | 0.36 |
| Down | T96L | 1061 | 2337.67 | 3156.45 | 0.74 | 0.37 | 0.51 |
| Down | T96W | 1058 | 1194.79 | 1631.19 | 0.73 | 0.19 | 0.26 |
| Down | T96N | 1051 | 2674.35 | 3874.07 | 0.69 | 0.43 | 0.62 |
| Neutral | T96G | 1055 | 415.04 | 387.45 | 1.07 | 0.07 | 0.06 |
| Down | T96F | 1056 | 2640.74 | 3897.10 | 0.68 | 0.42 | 0.62 |
| Down | T96Q | 1052 | 1865.64 | 2509.48 | 0.74 | 0.30 | 0.40 |
| Down | T96H | 1047 | 1294.29 | 1620.22 | 0.80 | 0.21 | 0.26 |
| Down | T96V | 1060 | 1904.14 | 2730.27 | 0.70 | 0.30 | 0.44 |
| Down | T961 | 1059 | 1814.91 | 2921.26 | 0.62 | 0.29 | 0.47 |
| Neutral | T96C | 1050 | 701.03 | 774.48 | 0.91 | 0.11 | 0.12 |
| Neutral | Y97R | 1068 | 447.48 | 449.81 | 0.99 | 0.14 | 0.09 |
| Neutral | Y97V | 1079 | 637.70 | 789.90 | 0.81 | 0.20 | 0.16 |
| Neutral | Y97A | 1081 | 507.18 | 504.63 | 1.01 | 0.16 | 0.10 |
| Neutral | Y97P | 1082 | 488.40 | 452.67 | 1.08 | 0.15 | 0.09 |
| Neutral | Y97L | 1080 | 510.25 | 549.53 | 0.93 | 0.16 | 0.11 |
| Neutral | Y97T | 1072 | 538.83 | 600.97 | 0.90 | 0.17 | 0.12 |
| Up | Y97K | 1067 | 469.55 | 390.08 | 1.20 | 0.15 | 0.08 |
| Down | Y97W | 1077 | 3115.45 | 4974.99 | 0.63 | 0.98 | 1.01 |
| Down | Y97H | 1066 | 685.71 | 879.64 | 0.78 | 0.22 | 0.18 |
| Neutral | Y97S | 1073 | 482.94 | 471.85 | 1.02 | 0.15 | 0.10 |
| Neutral | Y97E | 1065 | 435.12 | 432.07 | 1.01 | 0.14 | 0.09 |
| Neutral | Y97D | 1064 | 466.89 | 455.39 | 1.03 | 0.15 | 0.09 |
| Neutral | Y97N | 1070 | 486.98 | 490.84 | 0.99 | 0.15 | 0.10 |
| Neutral | Y97G | 1074 | 521.34 | 516.64 | 1.01 | 0.11 | 0.08 |
| Neutral | Y97Q | 1071 | 567.66 | 575.73 | 0.99 | 0.12 | 0.08 |
| Down | R9811 | 1085 | 1456.51 | 3257.97 | 0.45 | 0.46 | 0.66 |
| Down | R98K | 1086 | 2994.82 | 4670.17 | 0.64 | 0.95 | 0.95 |
| Neutral | R98C | 1087 | 761.34 | 938.43 | 0.81 | 0.24 | 0.19 |
| Down | R98L | 1099 | 3592.72 | 5087.24 | 0.71 | 1.14 | 1.03 |
| Down | R98M | 1095 | 3551.60 | 5834.31 | 0.61 | 1.12 | 1.18 |
| Down | R98F | 1094 | 2925.55 | 4988.31 | 0.59 | 0.92 | 1.01 |
| Down | R98W | 1096 | 833.68 | 1098.48 | 0.76 | 0.26 | 0.22 |
| Neutral | R98Y | 1091 | 505.91 | 479.02 | 1.06 | 0.16 | 0.10 |
| Down | R98P | 1101 | 2306.44 | 3388.72 | 0.68 | 0.73 | 0.69 |
| Down | R98E | 1084 | 1812.72 | 2769.07 | 0.65 | 0.57 | 0.56 |
| Down | R98A | 1100 | 3006.35 | 4371.72 | 0.69 | 0.95 | 0.89 |
| Down | R98G | 1093 | 1525.20 | 2367.66 | 0.64 | 0.48 | 0.48 |
| Down | R98V | 1098 | 1298.78 | 3330.10 | 0.39 | 0.26 | 0.49 |
| Down | R98S | 1092 | 4646.88 | 6142.58 | 0.76 | 0.94 | 0.90 |
| Down | R98D | 1083 | 2905.96 | 3867.31 | 0.75 | 0.33 | 0.47 |
| Neutral | I99C | 1107 | 514.61 | 514.66 | 1.00 | 0.16 | 0.10 |
| Neutral | I99E | 1103 | 550.80 | 548.33 | 1.00 | 0.17 | 0.11 |
| Neutral | I99G | 1113 | 588.17 | 598.60 | 0.98 | 0.19 | 0.12 |
| Neutral | I99H | 1104 | 749.01 | 834.15 | 0.90 | 0.24 | 0.17 |
| Neutral | I99N | 1108 | 691.55 | 805.73 | 0.86 | 0.22 | 0.16 |
| Neutral | I99P | 1120 | 567.03 | 526.02 | 1.08 | 0.18 | 0.11 |
| Down | I99T | 1110 | 1087.94 | 1583.58 | 0.69 | 0.34 | 0.32 |
| Down | I99V | 1117 | 2373.86 | 3390.37 | 0.70 | 0.75 | 0.69 |
| Neutral | I99A | 1119 | 654.22 | 809.57 | 0.81 | 0.13 | 0.12 |
| Down | I99F | 1114 | 2098.09 | 2958.45 | 0.71 | 0.43 | 0.43 |
| Down | I99L | 1118 | 2592.09 | 4336.89 | 0.60 | 0.53 | 0.63 |
| Neutral | I99R | 1106 | 561.16 | 555.21 | 1.01 | 0.11 | 0.08 |
| Neutral | I99S | 1112 | 616.13 | 673.46 | 0.91 | 0.12 | 0.10 |
| Down | I99Q | 1109 | 3318.21 | 4623.91 | 0.72 | 0.37 | 0.56 |
| Neutral | I99W | 1116 | 509.03 | 492.00 | 1.04 | 0.06 | 0.06 |
| Neutral | I99Y | 1111 | 690.55 | 700.48 | 0.99 | 0.08 | 0.09 |
| Down | E100V | 512 | 3980.72 | 5009.20 | 0.79 | 1.26 | 1.01 |
| Neutral | E100P | 515 | 727.82 | 785.14 | 0.93 | 0.23 | 0.16 |
| Down | E100L | 513 | 3370.21 | 4726.28 | 0.71 | 1.06 | 0.96 |
| Down | E100H | 498 | 1484.00 | 2354.50 | 0.63 | 0.47 | 0.48 |
| Down | E100D | 497 | 1886.86 | 3049.67 | 0.62 | 0.60 | 0.62 |
| Down | E100M | 509 | 3046.42 | 4566.62 | 0.67 | 0.96 | 0.92 |
| Neutral | E100G | 507 | 541.78 | 567.31 | 0.95 | 0.11 | 0.08 |
| Down | E100W | 510 | 1544.77 | 3766.06 | 0.41 | 0.31 | 0.55 |
| Down | E100Y | 505 | 2885.60 | 4167.75 | 0.69 | 0.58 | 0.61 |
| Neutral | E100R | 500 | 7410.11 | 7964.52 | 0.93 | 0.83 | 0.96 |
| Neutral | E100S | 506 | 3768.09 | 4664.58 | 0.81 | 0.42 | 0.56 |
| Neutral | E100T | 504 | 6985.28 | 7478.12 | 0.93 | 0.79 | 0.90 |
| Neutral | E100F | 508 | 6709.27 | 7436.60 | 0.90 | 0.75 | 0.90 |
| Neutral | E1001 | 511 | 8824.19 | 8458.79 | 1.04 | 0.99 | 1.02 |
| Neutral | E100N | 502 | 8809.68 | 8215.63 | 1.07 | 0.99 | 0.99 |
| Neutral | N101M | 1133 | 7907.75 | 7930.91 | 1.00 | 0.89 | 0.96 |
| Neutral | N101F | 1132 | 5045.4 | 5244.47 | 0.96 | 0.57 | 0.63 |
| Neutral | N101L | 1i37 | 6427.09 | 6656.60 | 0.97 | 0.72 | 0.80 |
| Neutral | N101V | 1136 | 8153.10 | 7605.57 | 1.07 | 0.92 | 0.92 |
| Neutral | N101H | 1123 | 8863.48 | 8197.03 | 1.08 | 1.00 | 0.99 |
| Neutral | N101R | 1125 | 8050.92 | 7576.48 | 1.06 | 0.91 | 0.91 |
| Down | N101C | 1126 | 2651.70 | 3359.06 | 0.79 | 0.30 | 0.41 |
| Neutral | N101T | 1128 | 9660.15 | 8437.52 | 1.14 | 1.09 | 1.02 |
| Neutral | N101P | 1139 | 8232.08 | 7996.53 | 1.03 | 0.93 | 0.96 |
| Neutral | N101W | 1134 | 3302.54 | 3773.04 | 0.88 | 0.37 | 0.46 |
| Neutral | N101K | 1124 | 7396.60 | 7283.35 | 1.02 | 0.83 | 0.88 |
| Neutral | N101S | 1130 | 8913.81 | 8187.44 | 1.09 | 1.00 | 0.99 |
| Neutral | N101D | 1121 | 5424.50 | 5676.43 | 0.96 | 0.61 | 0.68 |
| Neutral | N101A | 1138 | 6371.04 | 6423.71 | 0.99 | 0.72 | 0.78 |
| Neutral | N101Y | 1129 | 3878.66 | 3898.52 | 0.99 | 0.44 | 0.47 |
| Neutral | Y102R | 1144 | 9027.37 | 8170.55 | 1.10 | 1.02 | 0.99 |
| Neutral | Y102K | 1143 | 5806.60 | 5157.24 | 1.13 | 0.65 | 0.62 |
| Neutral | Y102V | 1155 | 6412.64 | 6500.28 | 0.99 | 0.72 | 0.78 |
| Neutral | Y102M | 1152 | 6668.55 | 6964.91 | 0.96 | 0.75 | 0.84 |
| Neutral | Y102P | 1158 | 4670.45 | 4431.46 | 1.05 | 0.53 | 0.53 |
| Neutral | Y102N | 1146 | 4618.85 | 4579.81 | 1.01 | 0.52 | 0.55 |
| Neutral | Y102G | 1150 | 7272.09 | 6976.34 | 1.04 | 0.82 | 0.84 |
| Neutral | Y102L | 1156 | 3323.14 | 3802.15 | 0.87 | 0.37 | 0.46 |
| Neutral | Y102D | 1140 | 5174.42 | 4862.10 | 1.06 | 0.58 | 0.59 |
| Neutral | Y102S | 1149 | 8744.32 | 8244.11 | 1.06 | 0.98 | 0.99 |
| Neutral | Y102F | 1151 | 7629.25 | 8362.12 | 0.91 | 0.86 | 1.01 |
| Neutral | Y102A | 1157 | 7177.10 | 7304.92 | 0.98 | 0.81 | 0.88 |
| Neutral | Y102E | 1141 | 3375.30 | 3325.21 | 1.02 | 0.38 | 0.40 |
| Neutral | Y102Q | 1147 | 5644.96 | 5711.44 | 0.99 | 0.64 | 0.69 |
| Neutral | Y102C | 1145 | 1544.43 | 1842.83 | 0.84 | 0.17 | 0.22 |
| Neutral | T103E | 517 | 619.06 | 617.97 | 1.00 | 0.20 | 0.13 |
| Neutral | T103D | 516 | 848.44 | 877.46 | 0.97 | 0.27 | 0.18 |
| Neutral | T103S | 525 | 761.49 | 855.80 | 0.89 | 0.24 | 0.17 |
| Up | T103L | 532 | 855.65 | 650.61 | 1.32 | 0.27 | 0.13 |
| Neutral | T103V | 531 | 822.60 | 1017.88 | 0.81 | 0.26 | 0.21 |
| Neutral | T103R | 520 | 674.37 | 652.99 | 1.03 | 0.21 | 0.13 |
| Neutral | T103Y | 524 | 1181.09 | 1423.76 | 0.83 | 0.37 | 0.29 |
| Down | T103N | 522 | 3131.62 | 4822.91 | 0.65 | 0.99 | 0.98 |
| Neutral | T103C | 521 | 628.62 | 604.98 | 1.04 | 0.20 | 0.12 |
| Up | T1O3Q | 523 | 791.61 | 624.86 | 1.27 | 0.25 | 0.13 |
| Neutral | T103W | 529 | 513.42 | 548.41 | 0.94 | 0.10 | 0.08 |
| Neutral | T103P | 534 | 513.57 | 526.91 | 0.97 | 0.10 | 0.08 |
| Neutral | T103A | 533 | 1058.92 | 950.05 | 1.11 | 0.21 | 0.14 |
| Neutral | T103G | 526 | 749.67 | 656.69 | 1.14 | 0.15 | 0.10 |
| Neutral | T103K | 519 | 884.09 | 777.94 | 1.14 | 0.18 | 0.11 |
| Neutral | P104G | 1170 | 602.57 | 620.78 | 0.97 | 0.19 | 0.13 |
| Down | P104E | 1160 | 4330.78 | 6029.01 | 0.72 | 1.37 | 1.22 |
| Down | P104T | 1167 | 3213.10 | 4681.67 | 0.69 | 1.02 | 0.95 |
| Neutral | P104F | 1171 | 2191.45 | 1923.19 | 1.14 | 0.69 | 0.39 |
| Down | P104R | 1163 | 591.46 | 5625.37 | 0.11 | 0.19 | 1.14 |
| Down | P104D | 1159 | 4022.87 | 5896.28 | 0.68 | 1.27 | 1.19 |
| Neutral | P104C | 1164 | 779.25 | 879.87 | 0.89 | 0.25 | 0.18 |
| Down | P104Q | 1166 | 4140.44 | 5971.62 | 0.69 | 1.31 | 1.21 |
| Down | P104V | 1175 | 2675.96 | 4161.77 | 0.64 | 0.85 | 0.84 |
| Down | P104Y | 1168 | 1907.52 | 2912.52 | 0.65 | 0.60 | 0.59 |
| Down | P104H | 1161 | 3404.74 | 5009.27 | 0.68 | 1.08 | 1.01 |
| Down | P104L | 1176 | 2981.52 | 4000.85 | 0.75 | 0.60 | 0.58 |
| Down | P104S | 1169 | 1205.11 | 2392.05 | 0.50 | 0.24 | 0.35 |
| Neutral | P104A | 1177 | 8861.30 | 8360.82 | 1.06 | 1.00 | 1.01 |
| Up | P104M | 1172 | 6709.44 | 7118.65 | 0.94 | 0.88 | 0.75 |
| Up | D105A | 39 | 2674.16 | 1227.06 | 2.18 | 0.65 | 0.24 |
| Neutral | D105C | 26 | 871.16 | 737.92 | 1.18 | 0.21 | 0.15 |
| Up | D105F | 33 | 2009.56 | 1221.58 | 1.65 | 0.49 | 0.24 |
| UP | D105G | 32 | 2407.89 | 1686.68 | 1.43 | 0.58 | 0.34 |
| Up | D105I | 36 | 1732.38 | 1105.99 | 1.57 | 0.42 | 0.22 |
| Up | D105L | 38 | 1563.61 | 859.56 | 1.82 | 0.38 | 0.17 |
| Neutral | D105M | 34 | 2703.51 | 2920.93 | 0.93 | 0.65 | 0.58 |
| Up | D105N | 27 | 3766.72 | 1475.08 | 2.55 | 0.91 | 0.29 |
| Up | D105P | 40 | 856.02 | 604.56 | 1.42 | 0.21 | 0.12 |
| Up | D105R | 25 | 3892.02 | 2016.90 | 1.93 | 0.94 | 0.40 |
| Up | D105S | 31 | 3646.49 | 2727.22 | 1.34 | 0.88 | 0.54 |
| Up | D105T | 29 | 2513.64 | 1729.46 | 1.45 | 0.61 | 0.34 |
| Neutral | D105V | 37 | 5824.43 | 6784.65 | 0.86 | 1.41 | 1.35 |
| Up | D105W | 35 | 2565.93 | 1855.05 | 1.38 | 0.62 | 0.37 |
| Neutral | D105E | 22 | 4000.92 | 3366.64 | 1.19 | 0.59 | 0.45 |
| Up | L106P | 1196 | 793.18 | 480.45 | 1.65 | 0.16 | 0.10 |
| Neutral | L106D | 1178 | 455.97 | 436.46 | 1.04 | 0.09 | 0.09 |
| Neutral | L106N | 1184 | 579.84 | 499.77 | 1.16 | 0.12 | 0.10 |
| Up | L106G | 1189 | 778.24 | 578.12 | 1.35 | 0.16 | 0.12 |
| Down | L106M | 1191 | 2299.74 | 3704.96 | 0.62 | 0.48 | 0.74 |
| Down | L106A | 1195 | 3604.47 | 5633.39 | 0.64 | 0.75 | 1.12 |
| Neutral | L106R | 1182 | 658.60 | 552.82 | 1.19 | 0.14 | 0.11 |
| Neutral | L106Y | 1187 | 4761.33 | 5769.09 | 0.83 | 0.99 | 1.15 |
| Neutral | L106T | 1186 | 1604.22 | 1508.31 | 1.06 | 0.33 | 0.30 |
| Neutral | L106V | 1194 | 8561.50 | 8230.68 | 1.04 | 1.77 | 1.64 |
| Neutral | L106H | 1180 | 644.13 | 641.84 | 1.00 | 0.13 | 0.13 |
| Down | L106F | 1190 | 1776.88 | 2525.65 | 0.70 | 0.36 | 0.37 |
| Down | L1061 | 1193 | 2787.16 | 4408.75 | 0.63 | 0.56 | 0.64 |
| Neutral | L106C | 1183 | 2995.56 | 3678.33 | 0.81 | 0.34 | 0.44 |
| Neutral | L106S | 1188 | 2730.64 | 2899.36 | 0.94 | 0.31 | 0.35 |
| Neutral | P107L | 1214 | 3183.54 | 3874.49 | 0.82 | 0.77 | 0.75 |
| Neutral | P107W | 1211 | 1255.79 | 1303.70 | 0.96 | 0.30 | 0.25 |
| Neutral | P107T | 1205 | 5673.07 | 6084.28 | 0.93 | 1.37 | 1.18 |
| Neutral | P107S | 1207 | 5865.31 | 6191.65 | 0.95 | 1.42 | 1.20 |
| Neutral | P107R | 1201 | 2981.87 | 3300.34 | 0.90 | 0.72 | 0.64 |
| Neutral | P107Y | 1206 | 2005.11 | 2383.15 | 0.84 | 0.48 | 0.46 |
| Neutral | P107M | 1210 | 3551.42 | 4031.55 | 0.88 | 0.86 | 0.78 |
| Neutral | P107V | 1213 | 3499.60 | 4142.87 | 0.84 | 0.85 | 0.80 |
| Neutral | P107D | 1197 | 3531.02 | 4095.17 | 0.86 | 0.85 | 0.80 |
| Neutral | P107A | 1215 | 5661.84 | 6316.88 | 0.90 | 1.37 | 1.23 |
| Neutral | P107C | 1202 | 786.68 | 776.20 | 1.01 | 0.19 | 0.15 |
| Neutral | P107K | 1200 | 3176.89 | 3653.27 | 0.87 | 0.77 | 0.71 |
| Neutral | P107F | 1209 | 1603.40 | 1832.50 | 0.87 | 0.39 | 0.36 |
| Neutral | P107I | 1212 | 2003.91 | 2369.36 | 0.85 | 0.48 | 0.46 |
| Neutral | P107G | 1208 | 2694.02 | 3272.63 | 0.82 | 0.65 | 0.64 |
| Up | R108P | 1234 | 4652.14 | 3388.90 | 1.37 | 0.96 | 0.67 |
| Down | R108G | 1226 | 4168.56 | 6204.10 | 0.67 | 0.86 | 1.24 |
| Neutral | R108T | 1223 | 1360.40 | 1652.92 | 0.82 | 0.28 | 0.33 |
| Down | R108E | 1217 | 5311.31 | 6829.34 | 0.78 | 1.10 | 1.36 |
| Down | R108A | 1233 | 5676.42 | 7183.19 | 0.79 | 1.18 | 1.43 |
| Down | R108Y | 1224 | 1527.69 | 2690.78 | 0.57 | 0.32 | 0.54 |
| Down | R108K | 1219 | 7212.78 | 9049.80 | 0.80 | 1.49 | 1.80 |
| Down | R108C | 1220 | 2092.15 | 2852.47 | 0.73 | 0.43 | 0.57 |
| Neutral | R108S | 1225 | 8515.31 | 8202.68 | 1.04 | 1.76 | 1.63 |
| Neutral | R108F | 1227 | 4264.07 | 5199.96 | 0.82 | 0.88 | 1.04 |
| Down | R108W | 1229 | 1522.39 | 2152.20 | 0.71 | 0.31 | 0.31 |
| Down | R1081 | 1230 | 2968.84 | 4628.28 | 0.64 | 0.60 | 0.68 |
| Down | R108L | 1232 | 2200.90 | 3462.10 | 0.64 | 0.45 | 0.51 |
| Down | R108N | 1221 | 2820.25 | 4415.19 | 0.64 | 0.57 | 0.65 |
| Neutral | R108V | 1231 | 571.77 | 618.30 | 0.92 | 0.12 | 0.09 |
| Neutral | A109S | 1245 | 6193.70 | 7627.42 | 0.81 | 1.28 | 1.52 |
| Down | A109R | 1239 | 4933.84 | 9751.06 | 0.51 | 1.02 | 1.94 |
| Down | A109T | 1243 | 4678.95 | 6089.37 | 0.77 | 0.97 | 1.21 |
| Down | A109W | 1249 | 5152.58 | 6447.41 | 0.80 | 1.07 | 1.28 |
| Down | A109I | 1250 | 2587.03 | 4255.55 | 0.61 | 0.54 | 0.85 |
| Down | A109Q | 1242 | 3475.21 | 4698.87 | 0.74 | 0.72 | 0.94 |
| Up | A109N | 1241 | 6266.66 | 4399.73 | 1.42 | 1.30 | 0.88 |
| Up | A109Y | 1244 | 1880.37 | 1444.85 | 1.30 | 0.39 | 0.29 |
| Down | A109G | 1246 | 5864.62 | 12111.28 | 0.48 | 1.21 | 2.41 |
| Neutral | A109M | 1248 | 7784.21 | 8628.31 | 0.90 | 1.61 | 1.72 |
| Down | A109D | 1235 | 4410.30 | 6431.60 | 0.69 | 0.91 | 1.28 |
| Neutral | A109V | 1251 | 8073.90 | 8388.34 | 0.96 | 1.67 | 1.67 |
| Down | A109E | 1236 | 2859.74 | 7453.25 | 0.38 | 0.58 | 1.09 |
| Down | A109L | 1252 | 3649.92 | 5241.27 | 0.70 | 0.74 | 0.77 |
| Neutral | A109H | 1237 | 7206.01 | 7536.96 | 0.96 | 0.81 | 0.91 |
| Down | D110P | 1272 | 691.78 | 937.18 | 0.74 | 0.14 | 0.19 |
| Down | D110F | 1265 | 2469.89 | 3158.71 | 0.78 | 0.51 | 0.63 |
| Down | D110Q | 1260 | 3028.40 | 4201.99 | 0.72 | 0.63 | 0.84 |
| Down | D110R | 1257 | 756.25 | 1109.97 | 0.68 | 0.16 | 0.22 |
| Neutral | D110M | 1266 | 1094.79 | 917.81 | 1.19 | 0.23 | 0.18 |
| Down | D110H | 1255 | 3327.99 | 6569.83 | 0.51 | 0.69 | 1.31 |
| Down | D1101 | 1268 | 1457.92 | 2219.69 | 0.66 | 0.30 | 0.44 |
| Down | D110L | 1270 | 1494.01 | 1991.44 | 0.75 | 0.31 | 0.40 |
| Down | D110V | 1269 | 2494.40 | 3413.88 | 0.73 | 0.52 | 0.68 |
| Down | D110T | 1261 | 2731.23 | 4170.98 | 0.65 | 0.57 | 0.83 |
| Down | D110S | 1263 | 1262.77 | 1714.94 | 0.74 | 0.26 | 0.34 |
| Down | D110Y | 1262 | 2764.78 | 5378.21 | 0.51 | 0.57 | 1.07 |
| Neutral | D110G | 1264 | 510.14 | 537.48 | 0.95 | 0.10 | 0.08 |
| Neutral | D110G | 1258 | 827.23 | 996.83 | 0.83 | 0.17 | 0.15 |
| Neutral | D110A | 1271 | 4179.59 | 5112.44 | 0.82 | 0.47 | 0.62 |
| Down | V111E | 1274 | 779.81 | 1134.36 | 0.69 | 0.16 | 0.23 |
| Down | V111A | 1290 | 1964.87 | 2890.23 | 0.68 | 0.41 | 0.58 |
| Down | V111S | 1283 | 2947.29 | 4188.33 | 0.70 | 0.61 | 0.83 |
| Neutral | V111W | 1287 | 601.19 | 580.46 | 1.04 | 0.12 | 0.12 |
| Neutral | V111G | 1284 | 833.15 | 974.76 | 0.85 | 0.17 | 0.19 |
| Neutral | V111Y | 1282 | 813.12 | 942.64 | 0.86 | 0.17 | 0.19 |
| Up | V111P | 1291 | 923.36 | 696.55 | 1.33 | 0.19 | 0.14 |
| Down | V111L | 1289 | 1070.50 | 1565.39 | 0.68 | 0.22 | 0.31 |
| Neutral | V111D | 1273 | 591.79 | 576.36 | 1.03 | 0.12 | 0.11 |
| Down | V111K | 1276 | 1017.96 | 1328.59 | 0.77 | 0.21 | 0.26 |
| Down | V111T | 1281 | 3551.97 | 4859.95 | 0.73 | 0.74 | 0.97 |
| Down | V111Q | 1280 | 1546.82 | 2061.98 | 0.75 | 0.32 | 0.41 |
| Down | V111I | 1288 | 4959.51 | 6699.66 | 0.74 | 1.03 | 1.33 |
| Neutral | V111C | 1278 | 843.17 | 943.89 | 0.89 | 0.17 | 0.14 |
| Neutral | V111R | 1277 | 2401.69 | 2925.16 | 0.82 | 0.27 | 0.35 |
| Down | D112A | 1309 | 1419.86 | 2167.48 | 0.66 | 0.29 | 0.43 |
| Down | D112M | 1304 | 1668.58 | 2249.91 | 0.74 | 0.35 | 0.45 |
| Down | D112V | 1307 | 2683.45 | 3699.41 | 0.73 | 0.56 | 0.74 |
| Down | D112R | 1295 | 1072.27 | 1395.54 | 0.77 | 0.22 | 0.28 |
| Down | D112K | 1294 | 967.53 | 1261.79 | 0.77 | 0.20 | 0.25 |
| Neutral | D112P | 1310 | 565.23 | 589.06 | 0.96 | 0.12 | 0.12 |
| Down | D112Q | 1298 | 4681.31 | 8975.21 | 0.52 | 0.97 | 1.79 |
| Down | D112F | 1303 | 1148.89 | 1477.74 | 0.78 | 0.24 | 0.29 |
| Down | D112G | 1302 | 1824.01 | 2601.95 | 0.70 | 0.38 | 0.52 |
| Neutral | D112C | 1296 | 866.83 | 1034.64 | 0.84 | 0.18 | 0.21 |
| Down | D112W | 1305 | 937.80 | 1277.50 | 0.73 | 0.19 | 0.25 |
| Neutral | D112T | 1299 | 2538.82 | 2941.38 | 0.86 | 0.53 | 0.59 |
| Neutral | D112H | 1293 | 480.11 | 467.40 | 1.03 | 0.10 | 0.07 |
| Neutral | D112S | 1301 | 7203.69 | 7600.93 | 0.95 | 0.81 | 0.92 |
| Down | D1121 | 1306 | 4020.53 | 5498.90 | 0.73 | 0.44 | 0.72 |
| Down | D112Y | 1300 | 2132.97 | 2869.86 | 0.74 | 0.23 | 0.38 |
| Down | D112L | 1308 | 2626.71 | 4159.92 | 0.63 | 0.29 | 0.55 |
| Neutral | H113T | 1318 | 9107.72 | 8278.01 | 1.10 | 1.03 | 1.00 |
| Neutral | H113L | 1327 | 9479.59 | 8454.16 | 1.12 | 1.07 | 1.02 |
| Neutral | H113M | 1323 | 9463.40 | 8759.43 | 1.08 | 1.07 | 1.06 |
| Neutral | H113S | 1320 | 9278.22 | 9159.47 | 1.01 | 1.04 | 1.11 |
| Neutral | H113N | 1316 | 8609.35 | 8502.46 | 1.01 | 0.97 | 1.03 |
| Neutral | H113R | 1314 | 7702.30 | 7852.46 | 0.98 | 0.87 | 0.95 |
| Neutral | H113A | 1328 | 8505.43 | 8090.18 | 1.05 | 0.96 | 0.98 |
| Neutral | H113E | 1312 | 9118.02 | 8443.69 | 1.08 | 1.03 | 1.02 |
| Neutral | H113V | 1326 | 9183.53 | 8450.30 | 1.09 | 1.03 | 1.02 |
| Neutral | H113Y | 1319 | 9688.60 | 8548.83 | 1.13 | 1.09 | 1.03 |
| Neutral | H113F | 1322 | 9472.51 | 8729.41 | 1.09 | 1.07 | 1.05 |
| Up | H113D | 1311 | 9304.42 | 4925.78 | 1.89 | 1.05 | 0.59 |
| Up | H113W | 1324 | 8683.10 | 5775.24 | 1.50 | 0.98 | 0.70 |
| Neutral | H113G | 1321 | 8953.60 | 8320.09 | 1.08 | 1.01 | 1.00 |
| Neutral | H113P | 1329 | 2987.12 | 3102.32 | 0.96 | 0.34 | 0.37 |
| Neutral | A114E | 1331 | 7136.25 | 7924.97 | 0.90 | 0.80 | 0.96 |
| Neutral | A114S | 1340 | 9211.05 | 8794.50 | 1.05 | 1.04 | 1.06 |
| Neutral | A114I | 1345 | 7073.18 | 7475.79 | 0.95 | 0.80 | 0.90 |
| Up | A114P | 1348 | 1691.05 | 1357.51 | 1.25 | 0.19 | 0.16 |
| Neutral | A114N | 1336 | 9250.51 | 8746.70 | 1.06 | 1.04 | 1.06 |
| Neutral | A114L | 1347 | 7749.61 | 8007.88 | 0.97 | 0.87 | 0.97 |
| Neutral | A114T | 1338 | 6242.22 | 6974.59 | 0.89 | 0.70 | 0.84 |
| Neutral | A114F | 1342 | 605.35 | 675.10 | 0.90 | 0.07 | 0.08 |
| Neutral | A114V | 1346 | 5527.85 | 6054.48 | 0.91 | 0.62 | 0.73 |
| Neutral | A114G | 1341 | 7663.26 | 7892.13 | 0.97 | 0.86 | 0.95 |
| Neutral | A114C | 1335 | 2412.52 | 3005.83 | 0.80 | 0.27 | 0.36 |
| Neutral | A114M | 1343 | 5287.05 | 5931.99 | 0.89 | 0.60 | 0.72 |
| Neutral | A114R | 1334 | 4454.65 | 3915.86 | 1.14 | 0.50 | 0.47 |
| Neutral | A114W | 1344 | 4654.58 | 5477.95 | 0.85 | 0.52 | 0.66 |
| Neutral | A114Q | 1337 | 8094.57 | 8337.94 | 0.97 | 0.91 | 1.01 |
| Neutral | I115F | 1361 | 9634.62 | 9011.34 | 1.07 | 1.08 | 1.09 |
| Neutral | I115T | 1357 | 1935.83 | 2379.92 | 0.81 | 0.22 | 0.29 |
| Neutral | I115H | 1351 | 805.66 | 825.35 | 0.98 | 0.09 | 0.10 |
| Neutral | I115G | 1360 | 725.85 | 626.12 | 1.16 | 0.08 | 0.08 |
| Down | I115K | 1352 | 642.87 | 920.32 | 0.70 | 0.07 | 0.11 |
| Neutral | I115E | 1350 | 1276.09 | 1211.16 | 1.05 | 0.14 | 0.15 |
| Neutral | I115S | 1359 | 796.93 | 780.25 | 1.02 | 0.09 | 0.09 |
| Neutral | I115P | 1367 | 626.77 | 597.01 | 1.05 | 0.07 | 0.07 |
| Neutral | I115C | 1354 | 1021.21 | 982.43 | 1.04 | 0.11 | 0.12 |
| Neutral | I115L | 1365 | 8869.57 | 8467.55 | 1.05 | 1.00 | 1.02 |
| Neutral | I115Q | 1356 | 732.25 | 652.35 | 1.12 | 0.08 | 0.08 |
| Up | I115R | 1353 | 750.11 | 575.36 | 1.30 | 0.08 | 0.07 |
| Neutral | I115W | 1363 | 2203.68 | 2304.27 | 0.96 | 0.25 | 0.28 |
| Neutral | I115V | 1364 | 9365.90 | 8785.30 | 1.07 | 1.05 | 1.06 |
| Neutral | I115D | 1349 | 694.17 | 641.97 | 1.08 | 0.08 | 0.08 |
| Neutral | E116A | 1385 | 9273.62 | 9051.39 | 1.02 | 1.04 | 1.09 |
| Neutral | E116C | 1372 | 5022.73 | 5732.42 | 0.88 | 0.57 | 0.69 |
| Neutral | E116D | 1368 | 9114.14 | 8594.45 | 1.06 | 1.03 | 1.04 |
| Neutral | E116F | 1379 | 8569.56 | 8473.84 | 1.01 | 0.96 | 1.02 |
| Neutral | E116G | 1378 | 8305.07 | 8358.04 | 0.99 | 0.94 | 1.01 |
| Neutral | E116H | 1369 | 8630.15 | 8386.63 | 1.03 | 0.97 | 1.01 |
| Neutral | E116I | 1382 | 9386.17 | 8740.84 | 1.07 | 1.06 | 1.05 |
| Neutral | E116K | 1370 | 9320.21 | 8760.44 | 1.06 | 1.05 | 1.06 |
| Neutral | E116L | 1384 | 8997.58 | 8736.18 | 1.03 | 1.01 | 1.05 |
| Neutral | E116M | 1380 | 9046.33 | 8478.98 | 1.07 | 1.02 | 1.02 |
| Neutral | E116N | 1373 | 8629.15 | 8503.39 | 1.01 | 0.97 | 1.03 |
| Neutral | E116P | 1386 | 852.91 | 806.00 | 1.06 | 0.10 | 0.10 |
| Neutral | E116Q | 1374 | 9480.67 | 8716.72 | 1.09 | 1.07 | 1.05 |
| Neutral | E116R | 1371 | 8871.32 | 8479.40 | 1.05 | 1.00 | 1.02 |
| Neutral | E116S | 1377 | 9714.89 | 8843.17 | 1.10 | 1.09 | 1.07 |
| Neutral | K117H | 1389 | 4516.51 | 4612.42 | 0.98 | 0.52 | 0.55 |
| Neutral | K117T | 1394 | 6149.94 | 6317.74 | 0.97 | 0.71 | 0.75 |
| Neutral | K117Q | 1393 | 6602.62 | 6024.00 | 1.10 | 0.77 | 0.72 |
| Neutral | K117E | 1388 | 668.03 | 667.32 | 1.00 | 0.08 | 0.08 |
| Neutral | K117A | 1404 | 7727.36 | 7375.30 | 1.05 | 0.90 | 0.88 |
| Neutral | K117F | 1398 | 4020.90 | 4038.83 | 1.00 | 0.47 | 0.48 |
| Neutral | K117D | 1387 | 5330.37 | 5924.02 | 0.90 | 0.62 | 0.70 |
| Down | K117N | 1392 | 4666.40 | 7745.69 | 0.60 | 0.54 | 0.92 |
| Neutral | K117G | 1397 | 7619.16 | 7218.94 | 1.06 | 0.88 | 0.86 |
| Neutral | K117W | 1400 | 5440.86 | 4780.56 | 1.14 | 0.63 | 0.57 |
| Neutral | K117Y | 1395 | 5047.23 | 4760.05 | 1.06 | 0.59 | 0.57 |
| Neutral | K117L | 1403 | 5277.39 | 5328.70 | 0.99 | 0.61 | 0.63 |
| Neutral | K117S | 1396 | 7278.89 | 6995.65 | 1.04 | 0.85 | 0.83 |
| Down | K117P | 1405 | 737.96 | 1153.03 | 0.64 | 0.09 | 0.14 |
| Neutral | K117R | 1390 | 8236.16 | 7677.40 | 1.07 | 0.96 | 0.91 |
| Down | A118G | 1417 | 2782.31 | 6427.69 | 0.43 | 0.32 | 0.76 |
| Neutral | A118R | 1410 | 4889.61 | 5639.79 | 0.87 | 0.57 | 0.67 |
| Up | A118W | 1420 | 652.55 | 465.07 | 1.40 | 0.08 | 0.06 |
| Neutral | A118K | 1409 | 584.59 | 543.84 | 1.07 | 0.07 | 0.06 |
| Neutral | A118P | 1424 | 883.04 | 810.72 | 1.09 | 0.10 | 0.10 |
| Neutral | A118V | 1422 | 869.06 | 754.10 | 1.15 | 0.10 | 0.09 |
| Neutral | A118L | 1423 | 543.99 | 523.84 | 1.04 | 0.06 | 0.06 |
| UP | A118D | 1406 | 617.40 | 468.39 | 1.32 | 0.07 | 0.06 |
| Down | A118S | 1416 | 5502.11 | 8251.39 | 0.67 | 0.64 | 0.98 |
| Neutral | A118F | 1418 | 7092.17 | 7315.30 | 0.97 | 0.82 | 0.87 |
| Up | A1181 | 1421 | 556.24 | 456.62 | 1.22 | 0.06 | 0.05 |
| Neutral | A118H | 1408 | 482.33 | 466.40 | 1.03 | 0.06 | 0.06 |
| Up | A118E | 1407 | 560.55 | 406.52 | 1.38 | 0.07 | 0.05 |
| Neutral | A118Q | 1413 | 517.05 | 477.18 | 1.08 | 0.06 | 0.06 |
| Neutral | A118T | 1414 | 745.83 | 665.63 | 1.12 | 0.13 | 0.13 |
| Down | F119G | 1436 | 2058.01 | 3284.43 | 0.63 | 0.24 | 0.39 |
| Down | F119T | 1433 | 4492.83 | 8234.56 | 0.55 | 0.52 | 0.98 |
| Neutral | F119R | 1429 | 648.01 | 665.21 | 0.97 | 0.08 | 0.08 |
| Neutral | F119L | 1441 | 8529.66 | 7666.52 | 1.11 | 0.99 | 0.91 |
| Neutral | F119N | 1431 | 1298.98 | 1614.30 | 0.80 | 0.15 | 0.19 |
| Down | F119S | 1435 | 3021.31 | 4383.36 | 0.69 | 0.35 | 0.52 |
| Neutral | F119C | 1430 | 2921.13 | 3375.91 | 0.87 | 0.34 | 0.40 |
| Neutral | F119P | 1443 | 567.10 | 665.80 | 0.85 | 0.07 | 0.08 |
| Neutral | F119W | 1438 | 4474.41 | 4610.60 | 0.97 | 0.52 | 0.55 |
| Neutral | F119K | 1428 | 679.32 | 762.81 | 0.89 | 0.08 | 0.09 |
| Down | F119H | 1427 | 2479.46 | 3939.67 | 0.63 | 0.29 | 0.47 |
| Neutral | F119A | 1442 | 7345.58 | 7881.39 | 0.93 | 0.85 | 0.94 |
| Neutral | F119V | 1440 | 7388.01 | 7712.75 | 0.96 | 0.86 | 0.92 |
| Neutral | F119Y | 1434 | 5832.62 | 6222.88 | 0.94 | 0.68 | 0.74 |
| Down | F119E | 1426 | 1044.34 | 1357.80 | 0.77 | 0.12 | 0.16 |
| Neutral | Q120K | 1447 | 8732.08 | 8385.78 | 1.04 | 1.01 | 1.00 |
| Neutral | Q120N | 1450 | 9186.34 | 8785.94 | 1.05 | 1.07 | 1.05 |
| Down | Q120A | 1461 | 613.72 | 979.68 | 0.63 | 0.07 | 0.12 |
| Neutral | Q120V | 1459 | 8711.16 | 8484.49 | 1.03 | 1.01 | 1.01 |
| Down | Q120D | 1444 | 5912.84 | 8887.98 | 0.67 | 0.69 | 1.06 |
| Neutral | Q120R | 1448 | 8845.48 | 8351.59 | 1.06 | 1.03 | 0.99 |
| Neutral | Q120P | 1462 | 1083.78 | 1186.17 | 0.91 | 0.13 | 0.14 |
| Neutral | Q120W | 1457 | 9339.94 | 7899.14 | 1.18 | 1.08 | 0.94 |
| Down | Q120Y | 1452 | 4891.24 | 8236.87 | 0.59 | 0.57 | 0.98 |
| Neutral | Q120C | 1449 | 5241.83 | 5502.66 | 0.95 | 0.61 | 0.65 |
| Neutral | Q120H | 1446 | 9155.83 | 8431.63 | 1.09 | 1.06 | 1.00 |
| Neutral | Q120T | 1451 | 9413.75 | 8645.01 | 1.09 | 1.09 | 1.03 |
| Down | Q120M | 1456 | 5740.33 | 8861.16 | 0.65 | 0.67 | 1.05 |
| Neutral | Q120E | 1445 | 8896.83 | 8424.76 | 1.06 | 1.03 | 1.00 |
| Neutral | Q120G | 1454 | 9176.97 | 8435.97 | 1.09 | 1.07 | 1.00 |
| Up | L121E | 1464 | 3183.74 | 2155.16 | 1.48 | 0.37 | 0.26 |
| Down | L121Q | 1470 | 2122.18 | 3128.11 | 0.68 | 0.25 | 0.37 |
| Neutral | L121P | 1481 | 1446.80 | 1342.92 | 1.08 | 0.17 | 0.16 |
| Up | L121R | 1467 | 1129.37 | 875.68 | 1.29 | 0.13 | 0.10 |
| Up | L121C | 1468 | 1592.13 | 1145.83 | 1.39 | 0.18 | 0.14 |
| Down | L121G | 1474 | 2613.21 | 4720.78 | 0.55 | 0.30 | 0.56 |
| Neutral | L121K | 1466 | 4678.64 | 4882.50 | 0.96 | 0.54 | 0.58 |
| Up | L121F | 1475 | 1227.52 | 957.08 | 1.28 | 0.14 | 0.11 |
| Neutral | L121I | 1478 | 7406.57 | 6937.72 | 1.07 | 0.86 | 0.83 |
| Down | L121S | 1473 | 2463.24 | 3614.68 | 0.68 | 0.29 | 0.43 |
| Neutral | L121V | 1479 | 7973.31 | 7244.01 | 1.10 | 0.93 | 0.86 |
| Up | L121H | 1465 | 3156.18 | 2605.48 | 1.21 | 0.37 | 0.31 |
| Neutral | L121T | 1471 | 7283.06 | 7372.13 | 0.99 | 0.85 | 0.88 |
| Down | L121A | 1480 | 3311.41 | 4989.77 | 0.66 | 0.38 | 0.59 |
| Neutral | L121N | 1469 | 6619.84 | 6504.79 | 1.02 | 0.77 | 0.77 |
| Neutral | W122R | 1486 | 651.20 | 598.41 | 1.09 | 0.08 | 0.07 |
| Neutral | W122A | 1499 | 699.90 | 617.84 | 1.13 | 0.08 | 0.07 |
| Neutral | W122N | 1488 | 484.17 | 598.30 | 0.81 | 0.06 | 0.07 |
| Neutral | W122P | 1500 | 619.39 | 605.42 | 1.02 | 0.07 | 0.07 |
| Neutral | W122T | 1490 | 621.86 | 570.65 | 1.09 | 0.07 | 0.07 |
| Neutral | W122L | 1498 | 580.35 | 563.09 | 1.03 | 0.07 | 0.07 |
| Neutral | W122G | 1493 | 602.75 | 646.94 | 0.93 | 0.07 | 0.08 |
| Neutral | W122S | 1492 | 602.28 | 564.94 | 1.07 | 0.07 | 0.07 |
| Neutral | W122V | 1497 | 607.75 | 532.36 | 1.14 | 0.07 | 0.06 |
| Neutral | W122H | 1484 | 596.81 | 545.92 | 1.09 | 0.07 | 0.06 |
| Down | W122F | 1494 | 2018.83 | 3056.56 | 0.66 | 0.23 | 0.36 |
| Neutral | W122Y | 1491 | 667.50 | 661.98 | 1.01 | 0.08 | 0.08 |
| Neutral | W122K | 1485 | 2724.60 | 2334.11 | 1.17 | 0.32 | 0.28 |
| Neutral | W122Q | 1489 | 576.75 | 528.48 | 1.09 | 0.07 | 0.06 |
| Neutral | W122E | 1483 | 564.38 | 580.16 | 0.97 | 0.07 | 0.07 |
| Neutral | S123D | 1501 | 9453.37 | 8830.71 | 1.07 | 0.92 | 0.94 |
| Neutral | S123L | 1517 | 9912.51 | 9431.98 | 1.05 | 0.97 | 1.01 |
| Neutral | S123A | 1518 | 9881.07 | 9237.14 | 1.07 | 0.97 | 0.99 |
| Neutral | S123C | 1506 | 10654.40 | 8973.60 | 1.19 | 1.04 | 0.96 |
| Neutral | S1231 | 1515 | 9679.91 | 8521.19 | 1.14 | 0.95 | 0.91 |
| Neutral | S123K | 1504 | 10567.78 | 9024.26 | 1.17 | 1.03 | 0.96 |
| Neutral | S123N | 1507 | 6481.00 | 5911.32 | 1.10 | 0.63 | 0.63 |
| Neutral | S123F | 1512 | 7485.79 | 8458.67 | 0.88 | 0.73 | 0.90 |
| Neutral | S123Y | 1510 | 7667.20 | 8806.19 | 0.87 | 0.75 | 0.94 |
| Neutral | S123M | 1513 | 9800.43 | 9159.15 | 1.07 | 0.96 | 0.98 |
| Neutral | S123H | 1503 | 10038.71 | 9099.05 | 1.10 | 0.98 | 0.97 |
| Down | S123R | 1505 | 5290.53 | 9248.50 | 0.57 | 0.52 | 0.99 |
| Down | S123W | 1514 | 2039.75 | 5970.03 | 0.34 | 0.20 | 0.64 |
| Down | S123T | 1509 | 5042.33 | 9146.80 | 0.55 | 0.49 | 0.98 |
| Neutral | S123P | 1519 | 884.66 | 799.56 | 1.11 | 0.09 | 0.09 |
| Neutral | S123G | 1511 | 10847.89 | 9512.32 | 1.14 | 1.06 | 1.02 |
| Neutral | S123Q | 1508 | 10841.56 | 9551.30 | 1.14 | 1.06 | 1.02 |
| Down | S123V | 1516 | 3220.29 | 4504.25 | 0.71 | 0.41 | 0.60 |
| Neutral | N124G | 1530 | 5601.70 | 6396.41 | 0.88 | 1.35 | 1.27 |
| Neutral | N124C | 1525 | 2241.39 | 2691.13 | 0.83 | 0.54 | 0.53 |
| Neutral | N124V | 1535 | 2966.25 | 3399.34 | 0.87 | 0.72 | 0.68 |
| Neutral | N124L | 1536 | 2342.22 | 2849.98 | 0.82 | 0.57 | 0.57 |
| Neutral | N124T | 1527 | 3872.37 | 4747.39 | 0.82 | 0.94 | 0.94 |
| Neutral | N124R | 1524 | 3795.95 | 4479.74 | 0.85 | 0.92 | 0.89 |
| Neutral | N124M | 1532 | 2818.81 | 3511.81 | 0.80 | 0.68 | 0.70 |
| Neutral | N124S | 1529 | 4245.94 | 5151.63 | 0.82 | 1.03 | 1.02 |
| Down | N124P | 1538 | 3825.40 | 5084.71 | 0.75 | 0.92 | 1.01 |
| Neutral | N124A | 1537 | 4174.53 | 4857.21 | 0.86 | 1.01 | 0.96 |
| Neutral | N124K | 1523 | 5006.93 | 5514.55 | 0.91 | 1.21 | 1.10 |
| Neutral | N124F | 1531 | 3681.53 | 4406.27 | 0.84 | 0.89 | 0.88 |
| Neutral | N124W | 1533 | 1506.21 | 1714.90 | 0.88 | 0.36 | 0.34 |
| Neutral | N1241 | 1534 | 1663.57 | 1830.11 | 0.91 | 0.40 | 0.36 |
| Neutral | N124D | 1520 | 6218.73 | 6620.92 | 0.94 | 0.92 | 0.88 |
| Neutral | V12SG | 1550 | 532.18 | 540.26 | 0.99 | 0.09 | 0.07 |
| Down | V125Q | 1546 | 1480.08 | 1883.56 | 0.79 | 0.26 | 0.25 |
| Down | V125S | 1549 | 2153.87 | 2966.73 | 0.73 | 0.38 | 0.39 |
| Down | VI25P | 1557 | 1410.46 | 1873.09 | 0.75 | 0.25 | 0.24 |
| Neutral | V125M | 1552 | 1056.84 | 1118.42 | 0.94 | 0.19 | 0.15 |
| Down | V12SY | 1548 | 1484.83 | 2214.89 | 0.67 | 0.26 | 0.29 |
| Down | V125T | 1547 | 1444.16 | 1850.94 | 0.78 | 0.25 | 0.24 |
| Down | V125A | 1556 | 3246.01 | 5558.20 | 0.58 | 0.57 | 0.73 |
| Up | V125C | 1544 | 892.38 | 690.11 | 1.29 | 0.16 | 0.09 |
| Neutral | V125D | 1539 | 727.50 | 723.91 | 1.00 | 0.13 | 0.09 |
| Neutral | V125W | 1553 | 1537.82 | 1638.09 | 0.94 | 0.27 | 0.21 |
| Neutral | V125R | 1543 | 1087.69 | 1057.82 | 1.03 | 0.19 | 0.14 |
| Neutral | V125E | 1540 | 1324.10 | 1545.82 | 0.86 | 0.23 | 0.20 |
| Down | V125F | 1551 | 1360.07 | 2068.15 | 0.66 | 0.24 | 0.27 |
| Neutral | V125H | 1541 | 2227.75 | 2720.50 | 0.82 | 0.39 | 0.36 |
| Neutral | T126K | 1561 | 646.69 | 546.31 | 1.18 | 0.16 | 0.11 |
| Down | T126V | 1573 | 3034.58 | 4559.28 | 0.67 | 0.73 | 0.91 |
| Neutral | T126G | 1568 | 970.67 | 820.16 | 1.18 | 0.23 | 0.16 |
| Neutral | T126R | 1562 | 692.68 | 612.62 | 1.13 | 0.17 | 0.12 |
| Neutral | T126L | 1574 | 1084.98 | 970.83 | 1.12 | 0.26 | 0.19 |
| Neutral | T126H | 1560 | 648.90 | 592.08 | 1.10 | 0.16 | 0.12 |
| Neutral | T126M | 1570 | 1168.66 | 1078.26 | 1.08 | 0.28 | 0.21 |
| Neutral | T126P | 1576 | 684.23 | 614.07 | 1.11 | 0.17 | 0.12 |
| Neutral | T126A | 1575 | 2433.37 | 2923.43 | 0.83 | 0.59 | 0.58 |
| Neutral | T126N | 1564 | 1449.19 | 1384.47 | 1.05 | 0.35 | 0.28 |
| Up | T126E | 1559 | 697.86 | 580.78 | 1.20 | 0.17 | 0.12 |
| Neutral | T126F | 1569 | 642.61 | 550.97 | 1.17 | 0.16 | 0.11 |
| Neutral | T126W | 1571 | 632.89 | 564.16 | 1.12 | 0.15 | 0.11 |
| Neutral | T126Q | 1565 | 664.00 | 591.91 | 1.12 | 0.16 | 0.12 |
| Neutral | T126S | 1567 | 7114.42 | 6856.69 | 1.04 | 1.06 | 0.91 |
| Neutral | P127C | 1582 | 1713.51 | 1846.56 | 0.93 | 0.41 | 0.37 |
| Neutral | P127F | 1589 | 1444.31 | 1603.37 | 0.90 | 0.35 | 0.32 |
| Neutral | P127T | 1585 | 2193.26 | 2519.16 | 0.87 | 0.53 | 0.50 |
| Down | P127E | 1578 | 2480.57 | 3177.56 | 0.78 | 0.60 | 0.63 |
| Neutral | P127W | 1591 | 1399.71 | 1476.35 | 0.95 | 0.34 | 0.29 |
| Neutral | P127A | 1595 | 1751.82 | 1662.47 | 1.05 | 0.42 | 0.33 |
| Neutral | P127S | 1587 | 2842.19 | 3070.41 | 0.93 | 0.69 | 0.61 |
| Up | P127H | 1579 | 2151.26 | 1693.77 | 1.27 | 0.52 | 0.34 |
| Neutral | P127Q | 1584 | 1729.40 | 1882.54 | 0.92 | 0.42 | 0.37 |
| Neutral | P127K | 1580 | 729.23 | 657.19 | 1.11 | 0.18 | 0.13 |
| Neutral | P127R | 1581 | 1590.44 | 1491.10 | 1.07 | 0.38 | 0.30 |
| Neutral | P1271 | 1592 | 1432.03 | 1464.78 | 0.98 | 0.35 | 0.29 |
| Neutral | P127V | 1593 | 1214.79 | 1401.27 | 0.87 | 0.29 | 0.28 |
| Neutral | P127L | 1594 | 1536.18 | 1604.60 | 0.96 | 0.37 | 0.32 |
| Neutral | P127M | 1590 | 2950.98 | 3052.79 | 0.97 | 0.71 | 0.61 |
| Neutral | L128F | 1608 | 1165.63 | 1269.01 | 0.92 | 0.28 | 0.25 |
| Neutral | L128M | 1609 | 1898.38 | 2135.63 | 0.89 | 0.46 | 0.42 |
| Neutral | L128T | 1604 | 756.63 | 698.21 | 1.08 | 0.18 | 0.14 |
| Neutral | L128R | 1600 | 919.42 | 960.28 | 0.96 | 0.22 | 0.19 |
| Neutral | L128S | 1606 | 764.28 | 672.98 | 1.14 | 0.18 | 0.13 |
| Neutral | L128G | 1607 | 738.26 | 694.65 | 1.06 | 0.18 | 0.14 |
| Neutral | L128I | 1611 | 1482.67 | 1715.03 | 0.86 | 0.36 | 0.34 |
| Neutral | L128Q | 1603 | 1042.55 | 936.43 | 1.11 | 0.25 | 0.19 |
| Neutral | L128P | 1614 | 792.57 | 760.45 | 1.04 | 0.19 | 0.15 |
| Neutral | L128A | 1613 | 769.15 | 712.50 | 1.08 | 0.19 | 0.14 |
| Neutral | L128D | 1596 | 682.02 | 642.58 | 1.06 | 0.16 | 0.13 |
| Down | L128V | 1612 | 1285.36 | 1696.88 | 0.76 | 0.31 | 0.34 |
| Neutral | L128W | 1610 | 776.89 | 664.61 | 1.17 | 0.19 | 0.13 |
| Neutral | L128C | 1601 | 856.43 | 770.10 | 1.11 | 0.21 | 0.15 |
| Neutral | L128K | 1599 | 858.27 | 846.02 | 1.01 | 0.21 | 0.17 |
| Neutral | T129G | 1625 | 4435.98 | 5356.41 | 0.83 | 1.07 | 1.06 |
| Down | T129A | 1632 | 2021.18 | 2774.35 | 0.73 | 0.49 | 0.55 |
| Neutral | T129C | 1620 | 1057.55 | 1033.96 | 1.02 | 0.26 | 0.21 |
| Neutral | T129K | 1618 | 3686.95 | 4446.15 | 0.83 | 0.89 | 0.88 |
| Down | T129F | 1626 | 2980.80 | 3803.12 | 0.78 | 0.72 | 0.76 |
| Neutral | T129Y | 1623 | 2527.88 | 2885.14 | 0.88 | 0.61 | 0.57 |
| Neutral | T129S | 1624 | 1649.34 | 1529.13 | 1.08 | 0.40 | 0.30 |
| Neutral | T129R | 1619 | 3334.95 | 3827.40 | 0.87 | 0.81 | 0.76 |
| Neutral | T129V | 1630 | 4967.86 | 5698.42 | 0.87 | 1.20 | 1.13 |
| Neutral | T129L | 1631 | 1649.57 | 1692.51 | 0.97 | 0.40 | 0.34 |
| Down | T129H | 1617 | 3019.81 | 3803.29 | 0.79 | 0.73 | 0.76 |
| Neutral | T129P | 1633 | 647.52 | 619.28 | 1.05 | 0.16 | 0.12 |
| Neutral | T129E | 1616 | 3205.16 | 3919.94 | 0.82 | 0.77 | 0.78 |
| Neutral | T1291 | 1629 | 3967.14 | 4452.39 | 0.89 | 0.96 | 0.88 |
| Down | T129M | 1627 | 4118.98 | 5214.21 | 0.79 | 1.00 | 1.04 |
| Neutral | F130L | 1650 | 1452.17 | 1651.58 | 0.88 | 0.25 | 0.23 |
| Neutral | F130P | 1652 | 703.36 | 797.39 | 0.88 | 0.12 | 0.11 |
| Neutral | F130C | 1639 | 803.88 | 939.50 | 0.86 | 0.14 | 0.13 |
| Neutral | F130R | 1638 | 613.42 | 687.58 | 0.89 | 0.10 | 0.09 |
| Down | F130Y | 1643 | 2355.45 | 3604.50 | 0.65 | 0.40 | 0.49 |
| Down | F130H | 1636 | 1209.72 | 1960.40 | 0.62 | 0.21 | 0.27 |
| Down | F130I | 1648 | 4480.99 | 5648.72 | 0.79 | 0.76 | 0.77 |
| Down | F130V | 1649 | 3403.91 | 4744.33 | 0.72 | 0.58 | 0.65 |
| Neutral | F130K | 1637 | 581.97 | 670.87 | 0.87 | 0.10 | 0.09 |
| Down | F130T | 1642 | 1529.21 | 2157.46 | 0.71 | 0.26 | 0.30 |
| Neutral | F130E | 1635 | 571.41 | 648.15 | 0.88 | 0.10 | 0.09 |
| Down | F130A | 1651 | 1414.89 | 1990.16 | 0.71 | 0.24 | 0.27 |
| Neutral | F130N | 1640 | 616.91 | 710.45 | 0.87 | 0.10 | 0.10 |
| Neutral | F130G | 1645 | 1553.35 | 1726.90 | 0.90 | 0.26 | 0.24 |
| Down | F130S | 1644 | 793.40 | 1055.93 | 0.75 | 0.13 | 0.14 |
| Down | T131F | 1664 | 2738.64 | 4500.49 | 0.61 | 0.48 | 0.59 |
| Neutral | T131P | 1671 | 540.49 | 640.19 | 0.84 | 0.10 | 0.08 |
| Down | T131A | 1670 | 3622.28 | 6028.39 | 0.60 | 0.64 | 0.79 |
| Down | T131S | 1662 | 3644.14 | 5779.25 | 0.63 | 0.64 | 0.75 |
| Down | T131G | 1663 | 3345.71 | 5523.72 | 0.61 | 0.59 | 0.72 |
| Down | T131I | 1667 | 2987.26 | 4570.78 | 0.65 | 0.53 | 0.60 |
| Down | T131L | 1669 | 3081.92 | 4518.80 | 0.68 | 0.54 | 0.59 |
| Down | T131H | 1655 | 4201.01 | 5298.03 | 0.79 | 0.74 | 0.69 |
| Down | TL31Q | 1660 | 6169.43 | 8400.64 | 0.73 | 1.08 | 1.10 |
| Neutral | TI31D | 1653 | 8629.30 | 9616.48 | 0.90 | 1.52 | 1.26 |
| Down | T131E | 1654 | 4396.59 | 6846.70 | 0.64 | 0.77 | 0.89 |
| Down | T131C | 1658 | 2232.15 | 3514.32 | 0.64 | 0.39 | 0.46 |
| Down | T131R | 1657 | 4325.73 | 6209.92 | 0.70 | 0.76 | 0.81 |
| Down | T131Y | 1661 | 2684.82 | 3916.43 | 0.69 | 0.47 | 0.51 |
| Down | T131M | 1665 | 3101.25 | 4674.29 | 0.66 | 0.55 | 0.61 |
| Down | K132G | 1682 | 3779.04 | 5835.32 | 0.65 | 0.64 | 0.80 |
| Down | K132V | 1687 | 3181.94 | 4834.70 | 0.66 | 0.54 | 0.66 |
| Down | K132L | 1688 | 2407.98 | 3744.64 | 0.64 | 0.41 | 0.51 |
| Down | K132A | 1689 | 5397.96 | 7468.39 | 0.72 | 0.92 | 1.02 |
| Down | K132P | 1690 | 4062.71 | 5742.05 | 0.71 | 0.69 | 0.79 |
| Down | K132F | 1683 | 2012.87 | 2934.12 | 0.69 | 0.34 | 0.40 |
| Neutral | K132R | 1675 | 7317.48 | 8467.31 | 0.86 | 1.24 | 1.16 |
| Down | K132I | 1686 | 1811.13 | 2747.29 | 0.66 | 0.31 | 0.38 |
| Down | K132H | 1674 | 3291.99 | 4588.07 | 0.72 | 0.56 | 0.63 |
| Neutral | K132S | 1681 | 4947.26 | 4913.96 | 1.01 | 0.84 | 0.67 |
| Down | K132M | 1684 | 4521.82 | 6773.06 | 0.67 | 0.77 | 0.93 |
| Down | K132D | 1672 | 2079.75 | 3166.80 | 0.66 | 0.35 | 0.43 |
| Down | K132T | 1679 | 2515.58 | 4096.35 | 0.61 | 0.43 | 0.56 |
| Down | K132Y | 1680 | 2363.32 | 3794.19 | 0.62 | 0.40 | 0.52 |
| Down | K132E | 1673 | 3617.16 | 5597.32 | 0.65 | 0.61 | 0.77 |
| Down | V133G | 1702 | 3203.88 | 5198.66 | 0.62 | 0.54 | 0.71 |
| Down | V133E | 1692 | 3621.55 | 5211.22 | 0.69 | 0.62 | 0.71 |
| Neutral | V133T | 1699 | 7931.49 | 8920.49 | 0.89 | 1.35 | 1.22 |
| Down | V133N | 1697 | 4321.90 | 6145.40 | 0.70 | 0.73 | 0.84 |
| Down | V133A | 1708 | 4764.29 | 6847.44 | 0.70 | 0.81 | 0.94 |
| Down | V133H | 1693 | 3351.37 | 4739.59 | 0.71 | 0.57 | 0.65 |
| Down | V133P | 1709 | 1405.33 | 2047.83 | 0.69 | 0.24 | 0.28 |
| Down | V133K | 1694 | 5737.27 | 7514.38 | 0.76 | 0.97 | 1.03 |
| Down | V133R | 1695 | 5773.82 | 7252.24 | 0.80 | 0.98 | 0.99 |
| Neutral | V133L | 1707 | 7039.08 | 8445.43 | 0.83 | 1.20 | 1.16 |
| Down | V133W | 1705 | 2475.35 | 3564.76 | 0.69 | 0.42 | 0.49 |
| Down | V133C | 1696 | 1863.63 | 2666.24 | 0.70 | 0.32 | 0.37 |
| Down | V133D | 1691 | 1792.46 | 2630.40 | 0.68 | 0.30 | 0.36 |
| Down | V133M | 1704 | 4618.45 | 6302.34 | 0.73 | 0.78 | 0.86 |
| Down | V133S | 1701 | 3077.53 | 4401.10 | 0.70 | 0.52 | 0.60 |
| Down | S134V | 1725 | 4041.51 | 5701.67 | 0.71 | 0.69 | 0.78 |
| Neutral | S134H | 1712 | 6079.44 | 7343.68 | 0.83 | 1.03 | 1.01 |
| Down | S134P | 1728 | 4779.98 | 6326.78 | 0.76 | 0.81 | 0.87 |
| Down | S134G | 1720 | 5540.33 | 7442.86 | 0.74 | 0.94 | 1.02 |
| Neutral | S134N | 1716 | 6292.89 | 7595.95 | 0.83 | 1.07 | 1.04 |
| Down | S134R | 1714 | 5129.73 | 6824.16 | 0.75 | 0.87 | 0.94 |
| Down | S134L | 1726 | 6015.18 | 8101.31 | 0.74 | 1.02 | 1.11 |
| Down | S134Q | 1717 | 4325.14 | 6159.02 | 0.70 | 0.73 | 0.84 |
| Neutral | S134E | 1711 | 7105.19 | 8577.53 | 0.83 | 1.21 | 1.18 |
| Down | 8134Y | 1719 | 5061.86 | 6645.94 | 0.76 | 0.86 | 0.91 |
| Down | S134A | 1727 | 5179.47 | 6920.72 | 0.75 | 0.88 | 0.95 |
| Down | S134K | 1713 | 5768.02 | 7876.85 | 0.73 | 0.98 | 1.08 |
| Neutral | S134D | 1710 | 6958.26 | 8316.41 | 0.84 | 1.18 | 1.14 |
| Down | S134T | 1718 | 5585.98 | 7301.80 | 0.77 | 0.95 | 1.00 |
| Down | S134C | 1715 | 1950.58 | 2625.09 | 0.74 | 0.33 | 0.36 |
| Down | E13SV | 1744 | 4036.77 | 5545.23 | 0.73 | 0.69 | 0.76 |
| Neutral | E135M | 1741 | 8700.42 | 9297.63 | 0.94 | 1.48 | 1.27 |
| Down | E135S | 1738 | 3895.80 | 5128.41 | 0.76 | 0.66 | 0.70 |
| Down | E135D | 1729 | 4858.77 | 6640.34 | 0.73 | 0.83 | 0.91 |
| Down | E13ST | 1736 | 4870.41 | 6518.41 | 0.75 | 0.83 | 0.89 |
| Down | E135L | 1745 | 3276.24 | 4342.02 | 0.75 | 0.56 | 0.59 |
| Down | E135A | 1746 | 5143.68 | 7429.20 | 0.69 | 0.87 | 1.02 |
| Down | E135W | 1742 | 3407.93 | 4761.19 | 0.72 | 0.58 | 0.65 |
| Down | E135F | 1740 | 3206.26 | 4561.41 | 0.70 | 0.54 | 0.63 |
| Down | E135P | 1747 | 1077.62 | 1567.43 | 0.69 | 0.18 | 0.21 |
| Neutral | E135R | 1732 | 815.91 | 921.41 | 0.89 | 0.14 | 0.13 |
| Down | E13SN | 1734 | 4626.44 | 6661.57 | 0.69 | 0.79 | 0.91 |
| Neutral | E135H | 1730 | 6074.22 | 7339.12 | 0.83 | 1.03 | 1.01 |
| Down | E135Q | 1735 | 5656.70 | 7144.49 | 0.79 | 0.96 | 0.98 |
| Down | E135I | 1743 | 2140.04 | 5232.08 | 0.41 | 0.36 | 0.72 |
| Down | G136V | 1763 | 1813.26 | 2616.78 | 0.69 | 0.31 | 0.36 |
| Down | G136W | 1761 | 993.10 | 1243.43 | 0.80 | 0.17 | 0.17 |
| Down | G136D | 1748 | 3591.52 | 5274.69 | 0.68 | 0.61 | 0.72 |
| Down | G136M | 1760 | 3515.40 | 5367.37 | 0.65 | 0.60 | 0.74 |
| Down | G136N | 1754 | 3503.65 | 5155.57 | 0.68 | 0.60 | 0.71 |
| Down | G136A | 1765 | 3559.58 | 5813.34 | 0.61 | 0.60 | 0.80 |
| Down | G136L | 1764 | 2187.68 | 3866.01 | 0.57 | 0.37 | 0.53 |
| Down | G136C | 1753 | 905.15 | 1515.22 | 0.60 | 0.15 | 0.21 |
| Down | G136P | 1766 | 3234.60 | 4934.89 | 0.66 | 0.55 | 0.68 |
| Down | G136T | 1756 | 2555.79 | 3746.36 | 0.68 | 0.43 | 0.51 |
| Down | G136R | 1752 | 2716.62 | 4398.06 | 0.62 | 0.46 | 0.60 |
| Down | G136S | 1758 | 3375.11 | 4670.21 | 0.72 | 0.57 | 0.64 |
| Down | G136I | 1762 | 2006.39 | 3604.58 | 0.56 | 0.34 | 0.49 |
| Down | G136H | 1750 | 3564.72 | 4804.54 | 0.74 | 0.61 | 0.66 |
| Down | G136E | 1749 | 5583.49 | 7289.45 | 0.77 | 0.95 | 1.00 |
| Down | Q137A | 1784 | 3966.25 | 6312.83 | 0.63 | 0.75 | 0.85 |
| Down | Q137R | 1771 | 3671.71 | 6256.44 | 0.59 | 0.69 | 0.84 |
| Down | Q137G | 1777 | 4573.46 | 6739.55 | 0.68 | 0.86 | 0.91 |
| Down | Q137K | 1770 | 6317.49 | 8133.50 | 0.78 | 1.19 | 1.09 |
| Down | Q137H | 1769 | 5645.75 | 7063.96 | 0.80 | 1.06 | 0.95 |
| Down | Q137P | 1785 | 5676.99 | 7744.50 | 0.73 | 1.07 | 1.04 |
| Down | Q137S | 1776 | 5384.46 | 7395.46 | 0.73 | 1.01 | 1.00 |
| Down | Q137L | 1783 | 5870.03 | 8003.53 | 0.73 | 1.10 | 1.08 |
| Down | Q137W | 1780 | 3200.77 | 5519.86 | 0.58 | 0.60 | 0.74 |
| Down | Q137F | 1778 | 3505.51 | 5883.52 | 0.60 | 0.66 | 0.79 |
| Down | Q137T | 1774 | 6636.95 | 8394.25 | 0.79 | 1.25 | 1.13 |
| Down | Q137C | 1772 | 1924.03 | 2898.49 | 0.66 | 0.36 | 0.39 |
| Down | Q137Y | 1775 | 5307.87 | 7091.79 | 0.75 | 1.00 | 0.95 |
| Down | Q137N | 1773 | 5369.70 | 7661.25 | 0.70 | 1.01 | 1.03 |
| Down | Q137E | 1768 | 5683.74 | 7333.42 | 0.78 | 1.07 | 0.99 |
| Down | A138V | 1802 | 1926.65 | 3043.75 | 0.63 | 0.36 | 0.41 |
| Neutral | A138L | 1803 | 594.07 | 675.23 | 0.88 | 0.11 | 0.09 |
| Down | A138P | 1804 | 1481.25 | 2478.85 | 0.60 | 0.28 | 0.33 |
| Down | A138C | 1791 | 1603.91 | 2981.97 | 0.54 | 0.30 | 0.40 |
| Down | A138T | 1794 | 1740.56 | 2785.40 | 0.62 | 0.33 | 0.37 |
| Down | A138S | 1796 | 2042.67 | 2909.34 | 0.70 | 0.38 | 0.39 |
| Down | A138R | 1790 | 759.61 | 962.30 | 0.79 | 0.14 | 0.13 |
| Down | A138G | 1797 | 3108.95 | 4692.85 | 0.66 | 0.58 | 0.63 |
| Down | A138E | 1787 | 1450.57 | 2644.44 | 0.55 | 0.27 | 0.36 |
| Down | A138H | 1788 | 667.58 | 839.37 | 0.80 | 0.13 | 0.11 |
| Neutral | A138M | 1799 | 626.50 | 749.79 | 0.84 | 0.12 | 0.10 |
| Down | A138Q | 1793 | 1747.09 | 2754.75 | 0.63 | 0.33 | 0.37 |
| Down | A138I | 1801 | 1984.57 | 3124.09 | 0.64 | 0.37 | 0.42 |
| Neutral | A138D | 1786 | 601.71 | 673.39 | 0.89 | 0.11 | 0.09 |
| Neutral | A138W | 1800 | 595.15 | 681.04 | 0.87 | 0.11 | 0.09 |
| Neutral | D139R | 1808 | 639.90 | 725.74 | 0.88 | 0.12 | 0.10 |
| Neutral | D139V | 1820 | 733.94 | 879.96 | 0.83 | 0.14 | 0.12 |
| Down | D139M | 1817 | 820.44 | 1093.19 | 0.75 | 0.15 | 0.15 |
| Neutral | D139C | 1809 | 763.17 | 886.67 | 0.86 | 0.14 | 0.12 |
| Down | D139P | 1823 | 794.89 | 1048.41 | 0.76 | 0.15 | 0.14 |
| Down | D139S | 1814 | 1060.53 | 1350.12 | 0.79 | 0.20 | 0.18 |
| Neutral | D139L | 1821 | 802.45 | 923.25 | 0.87 | 0.15 | 0.12 |
| Neutral | D1391 | 1819 | 759.45 | 884.92 | 0.86 | 0.14 | 0.12 |
| Down | D139H | 1806 | 1442.05 | 1944.83 | 0.74 | 0.27 | 0.26 |
| Down | D139A | 1822 | 899.50 | 1179.38 | 0.76 | 0.17 | 0.16 |
| Neutral | D139G | 1815 | 667.47 | 801.55 | 0.83 | 0.13 | 0.11 |
| Neutral | DI39F | 1816 | 670.14 | 828.73 | 0.81 | 0.13 | 0.11 |
| Down | D139N | 1810 | 1743.46 | 2795.27 | 0.62 | 0.33 | 0.38 |
| Neutral | D139W | 1818 | 641.04 | 769.42 | 0.83 | 0.12 | 0.10 |
| Neutral | D139Y | 1813 | 643.83 | 701.07 | 0.92 | 0.12 | 0.09 |
| Down | D139E | 1805 | 4365.22 | 7664.89 | 0.57 | 0.48 | 1.01 |
| Neutral | I140D | 1824 | 447.31 | 470.26 | 0.95 | 0.08 | 0.06 |
| Neutral | I140K | 1827 | 470.80 | 510.28 | 0.92 | 0.08 | 0.07 |
| Neutral | I140A | 1841 | 521.91 | 583.80 | 0.89 | 0.09 | 0.08 |
| Neutral | I140G | 1835 | 514.54 | 519.88 | 0.99 | 0.09 | 0.07 |
| Neutral | I140C | 1829 | 552.10 | 550.46 | 1.00 | 0.10 | 0.07 |
| Neutral | I140Y | 1833 | 476.67 | 511.05 | 0.93 | 0.08 | 0.07 |
| Down | I140V | 1839 | 1483.60 | 2240.10 | 0.66 | 0.26 | 0.29 |
| Neutral | I140W | 1838 | 541.30 | 540.55 | 1.00 | 0.10 | 0.07 |
| Neutral | I140F | 1836 | 671.22 | 710.61 | 0.94 | 0.12 | 0.09 |
| Neutral | I140H | 1826 | 568.54 | 584.67 | 0.97 | 0.10 | 0.08 |
| Down | I140L | 1840 | 4551.18 | 6862.42 | 0.66 | 0.80 | 0.90 |
| Neutral | I140R | 1828 | 479.35 | 467.38 | 1.03 | 0.08 | 0.06 |
| Neutral | I140E | 1825 | 480.00 | 481.92 | 1.00 | 0.08 | 0.06 |
| Down | I140M | 1837 | 1888.65 | 2695.29 | 0.70 | 0.33 | 0.35 |
| Neutral | I140T | 1832 | 493.59 | 505.63 | 0.98 | 0.09 | 0.07 |
| Down | M141E | 1844 | 2661.78 | 3381.08 | 0.79 | 0.64 | 0.66 |
| Neutral | M141I | 1857 | 3206.64 | 3834.28 | 0.84 | 0.77 | 0.74 |
| Neutral | M141R | 1847 | 3645.85 | 4050.91 | 0.90 | 0.88 | 0.79 |
| Neutral | M141T | 1851 | 1916.97 | 2186.65 | 0.88 | 0.46 | 0.42 |
| Neutral | M141P | 1861 | 957.33 | 1027.91 | 0.93 | 0.23 | 0.20 |
| Neutral | M141S | 1853 | 2578.82 | 3190.11 | 0.81 | 0.62 | 0.62 |
| Neutral | M141C | 1848 | 1162.41 | 1355.08 | 0.86 | 0.28 | 0.26 |
| Down | M141L | 1859 | 3257.69 | 4218.46 | 0.77 | 0.79 | 0.82 |
| Down | M141A | 1860 | 2798.52 | 3561.95 | 0.79 | 0.68 | 0.69 |
| Down | M141D | 1843 | 1943.58 | 2586.12 | 0.75 | 0.47 | 0.50 |
| Neutral | M141W | 1856 | 3860.87 | 4822.22 | 0.80 | 0.93 | 0.94 |
| Neutral | M141G | 1854 | 1252.97 | 1525.59 | 0.82 | 0.30 | 0.30 |
| Neutral | M141H | 1845 | 2221.73 | 2624.41 | 0.85 | 0.54 | 0.51 |
| Neutral | M141Y | 1852 | 2117.90 | 2326.92 | 0.91 | 0.51 | 0.45 |
| Neutral | M141N | 1849 | 3446.02 | 4175.18 | 0.83 | 0.83 | 0.81 |
| Down | I142L | 1878 | 4079.65 | 6338.98 | 0.64 | 0.72 | 0.83 |
| Down | I142M | 1875 | 2514.92 | 3872.66 | 0.65 | 0.44 | 0.51 |
| Neutral | I142G | 1873 | 590.45 | 573.21 | 1.03 | 0.10 | 0.07 |
| Neutral | I142K | 1865 | 567.58 | 566.13 | 1.00 | 0.10 | 0.07 |
| Down | I142A | 1879 | 1102.77 | 1776.73 | 0.62 | 0.19 | 0.23 |
| Neutral | I142N | 1868 | 544.78 | 582.84 | 0.93 | 0.10 | 0.08 |
| Neutral | I142W | 1876 | 614.88 | 660.20 | 0.93 | 0.11 | 0.09 |
| Neutral | I142P | 1880 | 517.98 | 553.16 | 0.94 | 0.09 | 0.07 |
| Neutral | I142Q | 1869 | 561.05 | 579.03 | 0.97 | 0.10 | 0.08 |
| Neutral | I142Y | 1871 | 535.36 | 568.63 | 0.94 | 0.09 | 0.07 |
| Down | I142V | 1877 | 2412.99 | 3835.99 | 0.63 | 0.42 | 0.50 |
| Neutral | I142T | 1870 | 619.92 | 700.51 | 0.88 | 0.11 | 0.09 |
| Neutral | I142R | 1866 | 592.22 | 631.35 | 0.94 | 0.10 | 0.08 |
| Neutral | I142S | 1872 | 560.11 | 608.47 | 0.92 | 0.10 | 0.08 |
| Down | I142F | 1874 | 988.49 | 1616.93 | 0.61 | 0.17 | 0.21 |
| Neutral | S143P | 1899 | 681.44 | 714.91 | 0.95 | 0.13 | 0.10 |
| Down | S143C | 1886 | 1242.25 | 1638.96 | 0.76 | 0.23 | 0.22 |
| Neutral | S143E | 1882 | 679.14 | 698.14 | 0.97 | 0.13 | 0.09 |
| Down | S143G | 1891 | 2178.31 | 3221.72 | 0.68 | 0.41 | 0.43 |
| Down | S143H | 1883 | 1946.43 | 3055.74 | 0.64 | 0.37 | 0.41 |
| Down | S143R | 1885 | 5284.60 | 7026.38 | 0.75 | 0.99 | 0.95 |
| Down | S143L | 1897 | 1855.11 | 3143.09 | 0.59 | 0.35 | 0.42 |
| Down | S143Q | 1888 | 4008.07 | 5922.69 | 0.68 | 0.75 | 0.80 |
| Down | S143N | 1887 | 3447.12 | 4827.78 | 0.71 | 0.65 | 0.65 |
| Neutral | S143W | 1894 | 1164.49 | 1414.47 | 0.82 | 0.22 | 0.19 |
| Down | S143A | 1898 | 4862.16 | 6797.05 | 0.72 | 0.91 | 0.91 |
| Down | S143T | 1889 | 3510.83 | 4873.23 | 0.72 | 0.66 | 0.66 |
| Down | S143Y | 1890 | 2566.36 | 3755.42 | 0.68 | 0.48 | 0.51 |
| Down | S143M | 1893 | 3680.60 | 6112.93 | 0.60 | 0.69 | 0.82 |
| Neutral | S1431 | 1895 | 6798.46 | 8447.06 | 0.80 | 1.28 | 1.14 |
| Neutral | F144K | 1903 | 683.92 | 723.08 | 0.95 | 0.13 | 0.10 |
| Neutral | F144M | 1912 | 727.58 | 785.61 | 0.93 | 0.14 | 0.11 |
| Neutral | F144E | 1901 | 684.55 | 697.38 | 0.98 | 0.13 | 0.09 |
| Neutral | F1445 | 1910 | 711.27 | 779.76 | 0.91 | 0.13 | 0.10 |
| Neutral | F144L | 1916 | 668.14 | 725.88 | 0.92 | 0.13 | 0.10 |
| Down | F144W | 1913 | 3272.56 | 4375.61 | 0.75 | 0.62 | 0.59 |
| Neutral | F144P | 1918 | 658.07 | 729.61 | 0.90 | 0.12 | 0.10 |
| Neutral | F144R | 1904 | 633.36 | 704.49 | 0.90 | 0.12 | 0.09 |
| Neutral | F144N | 1906 | 648.28 | 686.56 | 0.94 | 0.12 | 0.09 |
| Neutral | F144C | 1905 | 656.21 | 698.34 | 0.94 | 0.12 | 0.09 |
| Neutral | F144G | 1911 | 641.29 | 662.20 | 0.97 | 0.12 | 0.09 |
| Neutral | F144T | 1908 | 704.60 | 804.24 | 0.88 | 0.13 | 0.11 |
| Neutral | F144Q | 1907 | 679.28 | 759.34 | 0.89 | 0.13 | 0.10 |
| Neutral | F144H | 1902 | 766.54 | 861.04 | 0.89 | 0.14 | 0.12 |
| Neutral | F144V | 1915 | 664.73 | 737.66 | 0.90 | 0.12 | 0.10 |
| Down | V145A | 1936 | 5042.62 | 7103.17 | 0.71 | 0.89 | 0.93 |
| Down | V145T | 1927 | 3518.22 | 5408.09 | 0.65 | 0.62 | 0.71 |
| Down | V145L | 1935 | 4048.83 | 6522.67 | 0.62 | 0.71 | 0.85 |
| Down | V145P | 1937 | 2148.04 | 3271.70 | 0.66 | 0.38 | 0.43 |
| Down | V145K | 1922 | 4566.52 | 6542.14 | 0.70 | 0.80 | 0.85 |
| Down | V145N | 1925 | 5756.42 | 8553.91 | 0.67 | 1.01 | 1.12 |
| Down | V145D | 1919 | 3249.52 | 5915.18 | 0.55 | 0.57 | 0.77 |
| Down | V145H | 1921 | 3868.79 | 6370.16 | 0.61 | 0.68 | 0.83 |
| Down | V145R | 1923 | 5093.69 | 7494.19 | 0.68 | 0.90 | 0.98 |
| Down | V145Q | 1926 | 4550.79 | 6385.09 | 0.71 | 0.80 | 0.83 |
| Down | V145S | 1929 | 5229.00 | 7486.54 | 0.70 | 0.92 | 0.98 |
| Down | V145G | 1930 | 2139.70 | 3072.06 | 0.70 | 0.38 | 0.40 |
| Down | V145W | 1933 | 1735.30 | 3046.73 | 0.57 | 0.31 | 0.40 |
| Down | V145C | 1924 | 1652.16 | 3231.89 | 0.51 | 0.29 | 0.42 |
| Down | V145E | 1920 | 4086.60 | 6893.09 | 0.59 | 0.72 | 0.90 |
| Down | R146T | 1945 | 4145.84 | 6737.53 | 0.62 | 0.78 | 0.91 |
| Down | R146L | 1954 | 2149.16 | 3444.38 | 0.62 | 0.40 | 0.46 |
| Down | R146N | 1943 | 4441.83 | 6346.03 | 0.70 | 0.83 | 0.85 |
| Neutral | R146H | 1940 | 2791.26 | 3298.03 | 0.85 | 0.52 | 0.44 |
| Down | R146Q | 1944 | 4232.35 | 6620.08 | 0.64 | 0.80 | 0.89 |
| Down | R146K | 1941 | 5360.91 | 7104.88 | 0.75 | 1.01 | 0.96 |
| Neutral | R146C | 1942 | 776.54 | 868.97 | 0.89 | 0.15 | 0.12 |
| Neutral | R146S | 1947 | 8627.00 | 9288.66 | 0.93 | 1.62 | 1.25 |
| Down | R146D | 1938 | 3803.07 | 5389.79 | 0.71 | 0.71 | 0.73 |
| Down | R146A | 1955 | 4939.79 | 6859.12 | 0.72 | 0.93 | 0.92 |
| Down | R146Y | 1946 | 3175.81 | 5447.89 | 0.58 | 0.60 | 0.73 |
| Neutral | R146P | 1956 | 3019.88 | 2923.15 | 1.03 | 0.57 | 0.39 |
| Down | R146V | 1953 | 3662.01 | 6193.78 | 0.59 | 0.69 | 0.83 |
| Down | R146E | 1939 | 2538.71 | 3832.21 | 0.66 | 0.48 | 0.52 |
| Down | R146F | 1949 | 1272.11 | 2074.69 | 0.61 | 0.24 | 0.28 |
| Neutral | G147R | 1961 | 7744.82 | 8412.88 | 0.92 | 1.45 | 1.25 |
| Neutral | G147F | 1968 | 7821.91 | 7899.45 | 0.99 | 1.47 | 1.18 |
| Neutral | G1471 | 1971 | 1451.75 | 1461.30 | 0.99 | 0.27 | 0.22 |
| Down | G147L | 1973 | 1325.33 | 1787.45 | 0.74 | 0.25 | 0.27 |
| Neutral | G147A | 1974 | 2288.45 | 2655.58 | 0.86 | 0.43 | 0.40 |
| Down | G147E | 1958 | 1802.97 | 2340.36 | 0.77 | 0.34 | 0.35 |
| Neutral | G147H | 1959 | 6234.09 | 7432.44 | 0.84 | 1.17 | 1.11 |
| Down | G147W | 1970 | 5140.21 | 6807.71 | 0.76 | 0.96 | 1.01 |
| Down | G147T | 1965 | 5531.13 | 7240.25 | 0.76 | 1.04 | 1.08 |
| Neutral | G147C | 1962 | 6950.51 | 7385.23 | 0.94 | 1.30 | 1.10 |
| Down | G147S | 1967 | 3071.28 | 3887.91 | 0.79 | 0.58 | 0.58 |
| Neutral | G147V | 1972 | 4516.19 | 5576.27 | 0.81 | 0.85 | 0.83 |
| Neutral | G147Q | 1964 | 6879.67 | 7686.98 | 0.89 | 1.29 | 1.14 |
| Neutral | G147M | 1969 | 6059.54 | 7381.69 | 0.82 | 1.14 | 1.10 |
| Up | G147P | 1975 | 494.94 | 392.93 | 1.26 | 0.07 | 0.05 |
| Neutral | D148R | 1979 | 5874.44 | 7069.98 | 0.83 | 1.10 | 1.05 |
| Down | D148I | 1990 | 4934.66 | 6621.95 | 0.75 | 0.93 | 0.99 |
| Neutral | D148T | 1983 | 5534.68 | 6527.74 | 0.85 | 1.04 | 0.97 |
| Neutral | D148G | 1986 | 5545.91 | 6487.49 | 0.85 | 1.04 | 0.97 |
| Neutral | D148L | 1992 | 1738.87 | 2136.70 | 0.81 | 0.33 | 0.32 |
| Neutral | D148V | 1991 | 4521.62 | 5307.74 | 0.85 | 0.85 | 0.79 |
| Neutral | D148A | 1993 | 7276.18 | 7955.06 | 0.91 | 1.36 | 1.18 |
| Down | D148W | 1989 | 3622.09 | 4894.24 | 0.74 | 0.68 | 0.73 |
| Neutral | D148P | 1994 | 7311.56 | 7404.67 | 0.99 | 1.37 | 1.10 |
| Neutral | D148S | 1985 | 3190.82 | 3936.89 | 0.81 | 0.60 | 0.59 |
| Down | D148K | 1978 | 2414.59 | 3115.25 | 0.78 | 0.45 | 0.46 |
| Down | D148E | 1976 | 2457.68 | 3171.30 | 0.77 | 0.46 | 0.47 |
| Neutral | D148M | 1988 | 928.12 | 1156.79 | 0.80 | 0.17 | 0.17 |
| Neutral | D148N | 1981 | 5136.96 | 5810.99 | 0.88 | 0.96 | 0.87 |
| Neutral | D148C | 1980 | 2617.98 | 3259.70 | 0.80 | 0.49 | 0.49 |
| Neutral | H149W | 2008 | 578.88 | 610.12 | 0.95 | 0.10 | 0.08 |
| Neutral | H149A | 2012 | 574.51 | 606.50 | 0.95 | 0.10 | 0.08 |
| Neutral | H149L | 2011 | 562.23 | 585.57 | 0.96 | 0.10 | 0.08 |
| Neutral | H149C | 1999 | 532.13 | 536.84 | 0.99 | 0.09 | 0.07 |
| Neutral | H149Q | 2001 | 547.46 | 565.60 | 0.97 | 0.10 | 0.07 |
| Neutral | H149T | 2002 | 545.99 | 567.99 | 0.96 | 0.10 | 0.07 |
| Neutral | H149Y | 2003 | 553.52 | 575.93 | 0.96 | 0.10 | 0.08 |
| Neutral | H149P | 2013 | 502.45 | 522.17 | 0.96 | 0.09 | 0.07 |
| Neutral | H149V | 2010 | 515.00 | 521.68 | 0.99 | 0.09 | 0.07 |
| Neutral | H149R | 1998 | 481.87 | 534.48 | 0.90 | 0.08 | 0.07 |
| Neutral | H149G | 2005 | 492.47 | 525.75 | 0.94 | 0.09 | 0.07 |
| Neutral | H149E | 1996 | 476.14 | 472.99 | 1.01 | 0.08 | 0.06 |
| Neutral | H149S | 2004 | 481.76 | 508.54 | 0.95 | 0.08 | 0.07 |
| Neutral | H149I | 2009 | 510.38 | 533.47 | 0.96 | 0.09 | 0.07 |
| Neutral | H149N | 2000 | 542.00 | 555.29 | 0.98 | 0.10 | 0.07 |
| Neutral | R150S | 50 | 4221.17 | 4687.08 | 0.90 | 0.58 | 0.66 |
| Neutral | R150E | 42 | 9557.47 | 8282.03 | 1.15 | 1.31 | 1.17 |
| Neutral | R150G | 51 | 10002.15 | 8470.68 | 1.18 | 1.37 | 1.19 |
| Neutral | R150M | 53 | 8614.46 | 8306.99 | 1.04 | 1.18 | 1.17 |
| Up | R150P | 59 | 2291.14 | 828.28 | 2.77 | 0.31 | 0.12 |
| Neutral | R150T | 48 | 9808.17 | 8294.42 | 1.18 | 1.35 | 1.17 |
| Neutral | R150W | 54 | 8373.53 | 7574.51 | 1.11 | 1.15 | 1.07 |
| Neutral | R150A | 58 | 10175.13 | 8554.82 | 1.19 | 1.40 | 1.20 |
| Neutral | R150N | 46 | 10191.05 | 8571.32 | 1.19 | 1.40 | 1.21 |
| Neutral | R150K | 44 | 9471.29 | 8346.99 | 1.13 | 1.30 | 1.18 |
| Neutral | R150L | 57 | 9751.98 | 8444.63 | 1.15 | 1.34 | 1.19 |
| Neutral | R150V | 56 | 6869.28 | 6604.61 | 1.04 | 1.20 | 1.30 |
| Neutral | R150D | 41 | 7230.41 | 6033.28 | 1.20 | 1.26 | 1.19 |
| Down | R150I | 55 | 3120.05 | 4082.34 | 0.76 | 0.39 | 0.55 |
| Neutral | R150H | 43 | 8281.04 | 8056.17 | 1.03 | 1.05 | 1.08 |
| Neutral | D150R | 63 | 576.24 | 545.21 | 1.06 | 0.11 | 0.08 |
| Neutral | D151F | 71 | 626.76 | 601.08 | 1.04 | 0.12 | 0.09 |
| Neutral | D151P | 78 | 670.23 | 610.90 | 1.10 | 0.13 | 0.09 |
| Neutral | D151W | 73 | 691.38 | 656.86 | 1.05 | 0.13 | 0.10 |
| Neutral | D151Q | 66 | 634.58 | 619.91 | 1.02 | 0.12 | 0.09 |
| Neutral | D151L | 76 | 638.24 | 627.06 | 1.02 | 0.12 | 0.09 |
| Neutral | D151S | 69 | 612.74 | 579.48 | 1.06 | 0.11 | 0.09 |
| Up | D151G | 70 | 1073.32 | 733.89 | 1.46 | 0.20 | 0.11 |
| Neutral | D151A | 77 | 635.33 | 608.12 | 1.04 | 0.12 | 0.09 |
| Neutral | D151N | 65 | 631.72 | 612.41 | 1.03 | 0.12 | 0.09 |
| Neutral | D151K | 62 | 648.63 | 635.47 | 1.02 | 0.12 | 0.09 |
| Neutral | D151Y | 68 | 744.90 | 724.43 | 1.03 | 0.14 | 0.11 |
| Neutral | D151V | 75 | 586.23 | 585.19 | 1.00 | 0.11 | 0.09 |
| Neutral | D151T | 67 | 589.61 | 587.04 | 1.00 | 0.11 | 0.09 |
| Up | D151M | 72 | 2945.18 | 605.81 | 4.86 | 0.55 | 0.09 |
| Neutral | N152G | 2024 | 9852.70 | 8326.15 | 1.18 | 1.35 | 1.17 |
| Neutral | N152C | 2019 | 6322.44 | 7849.36 | 0.81 | 0.87 | 1.11 |
| Neutral | N152F | 2025 | 9762.67 | 8643.34 | 1.13 | 1.34 | 1.22 |
| Neutral | N152L | 2030 | 9176.14 | 8510.93 | 1.08 | 1.26 | 1.20 |
| Neutral | N152P | 2032 | 7767.98 | 7907.66 | 0.98 | 1.07 | 1.11 |
| Neutral | N152R | 2018 | 8348.81 | 7893.25 | 1.06 | 1.14 | 1.11 |
| Down | N152H | 2016 | 3318.01 | 4257.74 | 0.78 | 0.46 | 0.60 |
| Neutral | N152T | 2021 | 7155.46 | 7180.94 | 1.00 | 0.98 | 1.01 |
| Neutral | N152Y | 2022 | 8343.23 | 7992.79 | 1.04 | 1.14 | 1.13 |
| Neutral | N152K | 2017 | 7868.59 | 7956.82 | 0.99 | 1.08 | 1.12 |
| Neutral | N152D | 2014 | 10221.41 | 8616.74 | 1.19 | 1.40 | 1.21 |
| Neutral | N152W | 2027 | 5717.25 | 7086.82 | 0.81 | 0.78 | 1.00 |
| Neutral | N152I | 2028 | 10161.44 | 8648.89 | 1.17 | 1.39 | 1.22 |
| Neutral | N152A | 2031 | 6669.94 | 5660.16 | 1.18 | 1.17 | 1.12 |
| Down | N152S | 2023 | 4607.85 | 8096.31 | 0.57 | 0.58 | 1.08 |
| Neutral | S153I | 549 | 2873.12 | 2619.74 | 1.10 | 0.39 | 0.37 |
| Neutral | S153R | 539 | 4799.14 | 4905.35 | 0.98 | 0.66 | 0.69 |
| Neutral | S153K | 538 | 1002.00 | 1199.78 | 0.84 | 0.14 | 0.17 |
| Down | S153C | 540 | 1934.36 | 3181.56 | 0.61 | 0.27 | 0.45 |
| Neutral | S153G | 545 | 6175.12 | 6148.70 | 1.00 | 0.85 | 0.87 |
| Neutral | S153H | 537 | 9759.94 | 8837.02 | 1.10 | 1.34 | 1.24 |
| Neutral | S153L | 551 | 1285.63 | 1575.63 | 0.82 | 0.18 | 0.22 |
| Neutral | S153V | 550 | 8993.77 | 8047.48 | 1.12 | 1.23 | 1.13 |
| Neutral | S153T | 543 | 10530.07 | 8798.72 | 1.20 | 1.44 | 1.24 |
| Neutral | S153P | 553 | 9442.29 | 8513.31 | 1.11 | 1.29 | 1.20 |
| Neutral | S153A | 552 | 644.02 | 569.42 | 1.13 | 0.09 | 0.08 |
| Neutral | S153F | 546 | 10583.60 | 8979.56 | 1.18 | 1.45 | 1.26 |
| Neutral | S153D | 535 | 8477.40 | 8662.71 | 0.98 | 1.16 | 1.22 |
| Neutral | S153Q | 542 | 6654.12 | 7947.98 | 0.84 | 0.91 | 1.12 |
| Neutral | S153Y | 544 | 10164.62 | 8758.66 | 1.16 | 1.39 | 1.23 |
| Neutral | P154V | 2049 | 1257.75 | 1273.72 | 0.99 | 0.24 | 0.19 |
| Up | P154W | 2047 | 3838.51 | 2992.41 | 1.28 | 0.72 | 0.45 |
| Neutral | P154L | 2050 | 5826.55 | 6782.07 | 0.86 | 1.09 | 1.01 |
| Neutral | P154C | 2038 | 3097.69 | 3692.51 | 0.84 | 0.58 | 0.55 |
| Neutral | PIS4S | 2043 | 7417.09 | 8143.14 | 0.91 | 1.39 | 1.21 |
| Up | P154K | 2036 | 2407.68 | 1639.28 | 1.47 | 0.45 | 0.24 |
| Neutral | P1541 | 2048 | 7298.30 | 7549.63 | 0.97 | 1.37 | 1.12 |
| Down | P154A | 2051 | 2043.76 | 2680.62 | 0.76 | 0.38 | 0.40 |
| Neutral | P1S4T | 2041 | 1763.73 | 2075.38 | 0.85 | 0.33 | 0.31 |
| Neutral | P154H | 2035 | 1072.51 | 1021.04 | 1.05 | 0.20 | 0.15 |
| Neutral | P154Y | 2042 | 946.74 | 834.91 | 1.13 | 0.18 | 0.12 |
| Neutral | P154N | 2039 | 1122.04 | 1229.36 | 0.91 | 0.21 | 0.18 |
| Neutral | P154F | 2045 | 845.38 | 757.86 | 1.12 | 0.16 | 0.11 |
| Neutral | P154R | 2037 | 1975.77 | 1915.36 | 1.03 | 0.37 | 0.29 |
| Neutral | P154Q | 2040 | 2228.56 | 2374.11 | 0.94 | 0.42 | 0.35 |
| Neutral | F155S | 89 | 894.19 | 833.57 | 1.07 | 0.17 | 0.12 |
| Neutral | F155T | 87 | 1137.71 | 1084.01 | 1.05 | 0.21 | 0.16 |
| Neutral | F155G | 90 | 807.68 | 718.82 | 1.12 | 0.15 | 0.11 |
| Neutral | F155N | 85 | 715.78 | 688.72 | 1.04 | 0.13 | 0.10 |
| Neutral | F155R | 83 | 702.49 | 695.27 | 1.01 | 0.13 | 0.10 |
| Neutral | F155W | 92 | 715.40 | 693.53 | 1.03 | 0.13 | 0.10 |
| Up | F155L | 95 | 1322.13 | 864.19 | 1.53 | 0.25 | 0.13 |
| Neutral | F155Q | 86 | 731.28 | 738.56 | 0.99 | 0.14 | 0.11 |
| Neutral | F155M | 91 | 8252.43 | 8163.55 | 1.01 | 1.55 | 1.22 |
| Neutral | F155E | 80 | 685.90 | 683.12 | 1.00 | 0.13 | 0.10 |
| Up | F155A | 96 | 1250.93 | 760.12 | 1.65 | 0.23 | 0.11 |
| Neutral | F155P | 97 | 666.89 | 658.85 | 1.01 | 0.13 | 0.10 |
| Neutral | F155V | 94 | 681.25 | 679.13 | 1.00 | 0.13 | 0.10 |
| Neutral | F155H | 81 | 696.34 | 683.06 | 1.02 | 0.13 | 0.10 |
| Neutral | F155Y | 88 | 676.73 | 629.34 | 1.08 | 0.13 | 0.09 |
| Up | D156H | 99 | 2722.09 | 2081.55 | 1.31 | 0.51 | 0.31 |
| Up | D156L | 114 | 2548.30 | 1597.53 | 1.60 | 0.48 | 0.24 |
| Neutral | D156E | 98 | 6300.50 | 6871.25 | 0.92 | 1.18 | 1.02 |
| Up | D156A | 115 | 2679.29 | 1734.45 | 1.54 | 0.50 | 0.26 |
| Up | D156W | 111 | 1575.39 | 1268.36 | 1.24 | 0.30 | 0.19 |
| Neutral | D156C | 102 | 2842.85 | 2704.37 | 1.05 | 0.53 | 0.40 |
| Neutral | D156P | 116 | 1002.13 | 998.80 | 1.00 | 0.19 | 0.15 |
| Up | D156V | 113 | 1400.88 | 766.80 | 1.83 | 0.26 | 0.11 |
| Up | D156K | 100 | 1292.89 | 966.62 | 1.34 | 0.24 | 0.14 |
| Neutral | D156S | 107 | 969.70 | 837.57 | 1.16 | 0.18 | 0.12 |
| Neutral | D156G | 108 | 794.14 | 709.60 | 1.12 | 0.15 | 0.11 |
| Up | D156T | 105 | 2871.09 | 1843.03 | 1.56 | 0.54 | 0.27 |
| Neutral | D156Y | 106 | 3406.50 | 3113.95 | 1.09 | 0.64 | 0.46 |
| Up | D156R | 101 | 2431.23 | 1545.89 | 1.57 | 0.46 | 0.23 |
| Up | D156M | 110 | 817.96 | 502.82 | 1.63 | 0.12 | 0.07 |
| Up | G157K | 2055 | 677.09 | 562.66 | 1.20 | 0.09 | 0.08 |
| Neutral | G157D | 2052 | 603.28 | 513.64 | 1.17 | 0.08 | 0.07 |
| Neutral | G157F | 2064 | 9535.19 | 8450.24 | 1.13 | 1.31 | 1.19 |
| Up | G157R | 2056 | 704.56 | 540.98 | 1.30 | 0.10 | 0.08 |
| Neutral | G157H | 2054 | 608.42 | 567.38 | 1.07 | 0.08 | 0.08 |
| Up | G157L | 2069 | 582.39 | 476.09 | 1.22 | 0.08 | 0.07 |
| Up | G157N | 2059 | 721.55 | 534.46 | 1.35 | 0.10 | 0.08 |
| Up | G157Y | 2062 | 654.13 | 541.41 | 1.21 | 0.09 | 0.08 |
| Up | G157S | 2063 | 924.62 | 596.70 | 1.55 | 0.13 | 0.08 |
| Up | G157T | 2061 | 669.55 | 551.99 | 1.21 | 0.09 | 0.08 |
| Up | G1S7A | 2070 | 861.29 | 552.54 | 1.56 | 0.12 | 0.08 |
| Up | G157Q | 2060 | 655.49 | 522.72 | 1.25 | 0.09 | 0.07 |
| Neutral | G157P | 2071 | 635.63 | 591.49 | 1.07 | 0.09 | 0.08 |
| Neutral | G157V | 2068 | 654.45 | 573.19 | 1.14 | 0.09 | 0.08 |
| Neutral | G157M | 2065 | 716.52 | 615.65 | 1.16 | 0.10 | 0.09 |
| Neutral | P158S | 2082 | 7974.07 | 7118.62 | 1.12 | 1.09 | 1.00 |
| Neutral | P158Y | 2081 | 7544.63 | 6885.81 | 1.10 | 1.03 | 0.97 |
| Neutral | P158R | 2076 | 7142.54 | 6214.55 | 1.15 | 0.98 | 0.88 |
| Up | P158L | 2089 | 9290.77 | 6775.04 | 1.37 | 1.27 | 0.95 |
| Neutral | P158V | 2088 | 10642.99 | 8919.30 | 1.19 | 1.46 | 1.26 |
| Up | P158C | 2077 | 6284.97 | 4792.49 | 1.31 | 0.86 | 0.67 |
| Neutral | P158A | 2090 | 9579.29 | 8514.00 | 1.13 | 1.31 | 1.20 |
| Up | P158W | 2086 | 5175.38 | 3078.22 | 1.68 | 0.71 | 0.43 |
| Neutral | P1581 | 2087 | 10312.96 | 8597.26 | 1.20 | 1.41 | 1.21 |
| Up | P158F | 2084 | 6595.54 | 4090.71 | 1.61 | 0.90 | 0.58 |
| Up | P158Q | 2079 | 10928.51 | 8709.20 | 1.25 | 1.50 | 1.23 |
| Neutral | P158T | 2080 | 4204.23 | 3507.76 | 1.20 | 0.53 | 0.47 |
| Neutral | P158G | 2083 | 6277.86 | 549627 | 1.14 | 0.79 | 0.73 |
| Neutral | P158K | 2075 | 6860.82 | 6680.30 | 1.03 | 0.87 | 0.89 |
| Neutral | P158N | 2078 | 3656.04 | 3874.48 | 0.94 | 0.46 | 0.52 |
| Up | P158D | 2072 | 8959.02 | 7355.10 | 1.22 | 0.98 | 0.96 |
| Neutral | G159R | 121 | 6441.49 | 5914.02 | 1.09 | 0.88 | 0.83 |
| Neutral | G159S | 127 | 6594.46 | 6573.14 | 1.00 | 0.90 | 0.93 |
| Neutral | G159Q | 124 | 3996.96 | 4391.10 | 0.91 | 0.55 | 0.62 |
| Neutral | G159P | 135 | 596.30 | 564.24 | 1.06 | 0.08 | 0.08 |
| Up | G159V | 132 | 2453.98 | 732.46 | 3.35 | 0.34 | 0.10 |
| Neutral | G159K | 120 | 554.74 | 515.44 | 1.08 | 0.08 | 0.07 |
| Neutral | G159A | 134 | 5157.14 | 4685.20 | 1.10 | 0.71 | 0.66 |
| Up | G159Y | 126 | 1029.19 | 752.76 | 1.37 | 0.14 | 0.11 |
| Neutral | G159E | 118 | 4327.74 | 4027.23 | 1.07 | 0.59 | 0.57 |
| Up | G159T | 125 | 5059.91 | 1734.12 | 2.92 | 0.69 | 0.24 |
| Up | G159M | 129 | 5905.06 | 4874.00 | 1.21 | 0.75 | 0.65 |
| Neutral | G159I | 131 | 5725.99 | 5357.20 | 1.07 | 0.72 | 0.72 |
| Neutral | G1S9W | 130 | 6787.40 | 6287.71 | 1.08 | 0.86 | 0.84 |
| Neutral | G159L | 133 | 8231.62 | 7638.64 | 1.08 | 1.04 | 1.02 |
| Neutral | G159C | 122 | 2897.77 | 3053.86 | 0.95 | 0.37 | 0.41 |
| Down | G160A | 2108 | 2080.01 | 2823.12 | 0.74 | 0.60 | 0.58 |
| Down | G160H | 2093 | 2001.10 | 3085.22 | 0.65 | 0.57 | 0.63 |
| Down | G160N | 2097 | 3546.69 | 5339.11 | 0.66 | 1.02 | 1.09 |
| Neutral | G160W | 2104 | 4334.72 | 3946.12 | 1.10 | 1.24 | 0.80 |
| Down | G160R | 2095 | 2347.11 | 3791.36 | 0.62 | 0.67 | 0.77 |
| Neutral | G160P | 2109 | 1047.77 | 929.25 | 1.13 | 0.30 | 0.19 |
| Neutral | G160I | 2105 | 1794.48 | 1596.11 | 1.12 | 0.51 | 0.33 |
| Down | G160M | 2103 | 2506.95 | 3576.91 | 0.70 | 0.72 | 0.73 |
| Neutral | G160C | 2096 | 580.68 | 627.99 | 0.92 | 0.17 | 0.13 |
| Down | G160Q | 2098 | 4740.98 | 6839.68 | 0.69 | 1.36 | 1.39 |
| Neutral | G160V | 2106 | 3284.36 | 3030.37 | 1.08 | 0.94 | 0.62 |
| Down | G160S | 2101 | 2991.02 | 4281.08 | 0.70 | 0.86 | 0.87 |
| Neutral | G160E | 2092 | 3899.28 | 4071.63 | 0.96 | 1.12 | 0.83 |
| Down | G160L | 2107 | 3396.11 | 4411.61 | 0.77 | 0.97 | 0.90 |
| Neutral | G160T | 2099 | 3844.32 | 3943.21 | 0.97 | 1.10 | 0.80 |
| Down | N161S | 2119 | 1251.34 | 2417.50 | 0.52 | 0.36 | 0.49 |
| Down | N161C | 2115 | 1591.15 | 2710.58 | 0.59 | 0.46 | 0.55 |
| Down | N161L | 2126 | 4840.46 | 7275.68 | 0.67 | 1.39 | 1.48 |
| Neutral | N161R | 2114 | 6437.08 | 7915.53 | 0.81 | 1.85 | 1.61 |
| Down | N161G | 2120 | 3755.93 | 6728.88 | 0.56 | 1.08 | 1.37 |
| Down | N161W | 2123 | 3135.58 | 3994.07 | 0.79 | 0.90 | 0.81 |
| Down | N161Y | 2118 | 5507.52 | 7319.79 | 0.75 | 1.58 | 1.49 |
| Down | N161E | 2111 | 5410.44 | 8192.46 | 0.66 | 1.55 | 1.67 |
| Down | N161P | 2128 | 1231.02 | 1593.36 | 0.77 | 0.35 | 0.32 |
| Neutral | N161T | 2117 | 7464.29 | 9082.12 | 0.82 | 2.14 | 1.85 |
| Down | N161H | 2112 | 2727.71 | 5034.74 | 0.54 | 0.78 | 1.03 |
| Neutral | N161I | 2124 | 8070.21 | 9914.56 | 0.81 | 2.31 | 2.02 |
| Down | N161V | 2125 | 5238.40 | 7429.22 | 0.71 | 1.50 | 1.51 |
| Down | N161F | 2121 | 3890.45 | 6624.80 | 0.59 | 1.12 | 1.35 |
| Down | N161Q | 2116 | 4690.72 | 7412.73 | 0.63 | 1.35 | 1.51 |
| Neutral | L162A | 2146 | 584.17 | 505.94 | 1.15 | 0.17 | 0.10 |
| Neutral | L162G | 2140 | 582.48 | 602.57 | 0.97 | 0.17 | 0.12 |
| Neutral | L162C | 2134 | 475.91 | 466.85 | 1.02 | 0.14 | 0.10 |
| Neutral | L162P | 2147 | 514.26 | 519.71 | 0.99 | 0.15 | 0.11 |
| Neutral | L162R | 2133 | 492.19 | 498.99 | 0.99 | 0.14 | 0.10 |
| Down | L162I | 2144 | 2948.11 | 4018.68 | 0.73 | 0.85 | 0.82 |
| Neutral | L162S | 2139 | 473.63 | 459.28 | 1.03 | 0.14 | 0.09 |
| Neutral | L162D | 2129 | 512.72 | 487.18 | 1.05 | 0.15 | 0.10 |
| Neutral | L162M | 2142 | 1013.31 | 1138.86 | 0.89 | 0.29 | 0.23 |
| Neutral | L162E | 2i30 | 563.63 | 631.85 | 0.89 | 0.16 | 0.13 |
| Neutral | L162T | 2137 | 473.46 | 477.00 | 0.99 | 0.14 | 0.10 |
| Neutral | L162Y | 2138 | 484.26 | 519.58 | 0.93 | 0.14 | 0.11 |
| Neutral | L162F | 2141 | 484.37 | 469.30 | 1.03 | 0.14 | 0.10 |
| Neutral | L162W | 2143 | 463.12 | 457.12 | 1.01 | 0.13 | 0.09 |
| Neutral | L162Q | 2136 | 480.75 | 481.03 | 1.00 | 0.14 | 0.10 |
| Neutral | A163R | 2152 | 562.68 | 563.06 | 1.00 | 0.16 | 0.11 |
| Neutral | A163G | 2159 | 819.22 | 999.88 | 0.82 | 0.23 | 0.20 |
| Neutral | A163Y | 2157 | 562.12 | 549.69 | 1.02 | 0.16 | 0.11 |
| Neutral | A163P | 2166 | 557.10 | 559.72 | 1.00 | 0.16 | 0.11 |
| Neutral | A163S | 2158 | 572.70 | 542.75 | 1.06 | 0.16 | 0.11 |
| Neutral | A163L | 2165 | 532.98 | 539.88 | 0.99 | 0.15 | 0.11 |
| Neutral | A163C | 2153 | 528.01 | 546.64 | 0.97 | 0.15 | 0.11 |
| Neutral | A163K | 2151 | 510.99 | 502.63 | 1.02 | 0.15 | 0.10 |
| Neutral | A163V | 2164 | 567.33 | 572.66 | 0.99 | 0.16 | 0.12 |
| Down | A163F | 2160 | 931.85 | 1182.48 | 0.79 | 0.27 | 0.24 |
| Neutral | A163E | 2149 | 560.80 | 539.21 | 1.04 | 0.16 | 0.11 |
| Neutral | A163T | 2156 | 538.98 | 537.66 | 1.00 | 0.15 | 0.11 |
| Neutral | A163Q | 2155 | 586.94 | 586.24 | 1.00 | 0.17 | 0.12 |
| Neutral | A163I | 2163 | 554.29 | 579.47 | 0.96 | 0.16 | 0.12 |
| Neutral | A163N | 2154 | 575.87 | 580.49 | 0.99 | 0.17 | 0.12 |
| Neutral | H164L | 2183 | 547.21 | 565.25 | 0.97 | 0.16 | 0.12 |
| Neutral | H164M | 2179 | 552.91 | 590.51 | 0.94 | 0.16 | 0.12 |
| Neutral | H164K | 2169 | 575.53 | 589.33 | 0.98 | 0.17 | 0.12 |
| Neutral | H164P | 2185 | 573.34 | 570.59 | 1.00 | 0.16 | 0.12 |
| Neutral | H164C | 2171 | 551.45 | 576.69 | 0.96 | 0.16 | 0.12 |
| Neutral | H164R | 2170 | 558.91 | 553.87 | 1.01 | 0.16 | 0.11 |
| Neutral | H164A | 2184 | 549.93 | 598.96 | 0.92 | 0.16 | 0.12 |
| Neutral | H164V | 2182 | 567.08 | 579.35 | 0.98 | 0.16 | 0.12 |
| Down | H164S | 2176 | 4849.81 | 6939.34 | 0.70 | 1.39 | 1.41 |
| Down | H164N | 2172 | 437.45 | 585.42 | 0.75 | 0.13 | 0.12 |
| Neutral | H164G | 2177 | 545.54 | 547.89 | 1.00 | 0.16 | 0.11 |
| Neutral | H164F | 2178 | 540.67 | 537.69 | 1.01 | 0.16 | 0.11 |
| Neutral | H164Y | 2175 | 558.66 | 548.15 | 1.02 | 0.16 | 0.11 |
| Neutral | H164Q | 2173 | 566.62 | 555.39 | 1.02 | 0.16 | 0.11 |
| Neutral | H164E | 2168 | 569.92 | 612.16 | 0.93 | 0.16 | 0.12 |
| Neutral | A165W | 2200 | 583.56 | 591.99 | 0.99 | 0.17 | 0.12 |
| Neutral | A165V | 2202 | 560.38 | 564.19 | 0.99 | 0.16 | 0.11 |
| Down | A165G | 2197 | 445.09 | 575.94 | 0.77 | 0.13 | 0.12 |
| Neutral | A165K | 2189 | 537.18 | 537.57 | 1.00 | 0.15 | 0.11 |
| Neutral | A165L | 2203 | 552.58 | 553.45 | 1.00 | 0.16 | 0.11 |
| Neutral | A165P | 2204 | 535.50 | 554.41 | 0.97 | 0.15 | 0.11 |
| Down | A165Q | 2193 | 983.84 | 1344.06 | 0.73 | 0.28 | 0.27 |
| Neutral | A165D | 2186 | 534.17 | 577.13 | 0.93 | 0.15 | 0.12 |
| Neutral | A165H | 2188 | 515.90 | 536.85 | 0.96 | 0.15 | 0.11 |
| Neutral | A165F | 2198 | 493.42 | 496.39 | 0.99 | 0.14 | 0.10 |
| Down | A165S | 2196 | 390.18 | 578.00 | 0.68 | 0.11 | 0.12 |
| Neutral | A165T | 2194 | 506.15 | 502.78 | 1.01 | 0.15 | 0.10 |
| Neutral | A165R | 2190 | 485.08 | 477.19 | 1.02 | 0.14 | 0.10 |
| Neutral | A165N | 2192 | 509.08 | 499.01 | 1.02 | 0.15 | 0.10 |
| Neutral | A165M | 2199 | 473.24 | 523.60 | 0.90 | 0.14 | 0.11 |
| Neutral | F166G | 2216 | 623.89 | 586.56 | 1.06 | 0.11 | 0.08 |
| Neutral | F166S | 2215 | 724.53 | 695.67 | 1.04 | 0.12 | 0.10 |
| Neutral | F166L | 2221 | 760.25 | 829.02 | 0.92 | 0.13 | 0.12 |
| Neutral | F166V | 2220 | 552.68 | 564.25 | 0.98 | 0.09 | 0.08 |
| Neutral | F166P | 2223 | 530.80 | 562.94 | 0.94 | 0.09 | 0.08 |
| Neutral | F166N | 2211 | 613.07 | 589.89 | 1.04 | 0.10 | 0.08 |
| Neutral | F166R | 2209 | 534.62 | 543.15 | 0.98 | 0.09 | 0.08 |
| Neutral | F166A | 2222 | 638.77 | 712.81 | 0.90 | 0.11 | 0.10 |
| Neutral | F166K | 2208 | 598.42 | 615.59 | 0.97 | 0.10 | 0.09 |
| Neutral | F166H | 2207 | 2770.43 | 2606.89 | 1.06 | 0.47 | 0.37 |
| Neutral | F166W | 2218 | 8234.80 | 8549.89 | 0.96 | 1.40 | 1.20 |
| Neutral | F166I | 2219 | 617.86 | 613.36 | 1.01 | 0.10 | 0.09 |
| Neutral | F166M | 2217 | 537.05 | 571.21 | 0.94 | 0.09 | 0.08 |
| Neutral | F166C | 2210 | 661.10 | 639.33 | 1.03 | 0.11 | 0.09 |
| Neutral | F166E | 2206 | 616.99 | 582.48 | 1.06 | 0.10 | 0.08 |
| Neutral | Q167D | 2224 | 4883.56 | 4579.11 | 1.07 | 0.83 | 0.64 |
| Neutral | Q167R | 2228 | 7660.88 | 8025.72 | 0.95 | 1.30 | 1.12 |
| Neutral | Q167A | 2241 | 8466.37 | 8182.70 | 1.03 | 1.44 | 1.15 |
| Neutral | Q167S | 2233 | 7915.08 | 8512.14 | 0.93 | 1.34 | 1.19 |
| Neutral | Q167F | 2235 | 8209.07 | 8535.93 | 0.96 | 1.39 | 1.20 |
| Down | Q167Y | 2232 | 5687.17 | 7642.43 | 0.74 | 0.96 | 1.07 |
| Neutral | Q167P | 2242 | 7513.70 | 8011.11 | 0.94 | 1.27 | 1.12 |
| Neutral | Q167L | 2231 | 7772.59 | 8173.09 | 0.95 | 1.32 | 1.15 |
| Neutral | Q167V | 2239 | 7867.97 | 8191.44 | 0.96 | 1.33 | 1.15 |
| Neutral | Q167L | 2240 | 7174.37 | 7937.04 | 0.90 | 1.22 | 1.11 |
| Neutral | Q167M | 2236 | 8005.59 | 8974.74 | 0.89 | 1.36 | 1.26 |
| Down | Q167N | 2230 | 3612.25 | 5004.93 | 0.72 | 0.61 | 0.70 |
| Neutral | Q167G | 2234 | 6671.99 | 7782.93 | 0.86 | 1.13 | 1.09 |
| Neutral | Q167K | 2227 | 6453.33 | 7646.48 | 0.84 | 1.09 | 1.07 |
| Down | Q167E | 2225 | 5009.36 | 6388.75 | 0.78 | 0.85 | 0.90 |
| Neutral | P168N | 2249 | 638.46 | 590.71 | 1.08 | 0.11 | 0.08 |
| Neutral | P168F | 2255 | 673.09 | 638.53 | 1.05 | 0.11 | 0.09 |
| Neutral | P168R | 2247 | 7038.51 | 7902.50 | 0.89 | 1.19 | 1.11 |
| Neutral | P168W | 2257 | 737.53 | 666.57 | 1.11 | 0.13 | 0.09 |
| Neutral | P168A | 2261 | 833.88 | 736.90 | 1.13 | 0.14 | 0.10 |
| Neutral | P168T | 2251 | 646.42 | 645.28 | 1.00 | 0.11 | 0.09 |
| Neutral | P168V | 2259 | 499.02 | 557.37 | 0.90 | 0.08 | 0.08 |
| Neutral | P1680 | 2254 | 686.51 | 644.44 | 1.07 | 0.12 | 0.09 |
| Neutral | P168C | 2248 | 568.42 | 598.90 | 0.95 | 0.10 | 0.08 |
| Neutral | P168M | 2256 | 734.84 | 652.57 | 1.13 | 0.12 | 0.09 |
| Neutral | P168H | 2245 | 590.54 | 588.07 | 1.00 | 0.10 | 0.08 |
| Neutral | P168L | 2260 | 715.20 | 706.06 | 1.01 | 0.12 | 0.10 |
| Neutral | P168S | 2253 | 641.79 | 605.44 | 1.06 | 0.11 | 0.08 |
| Neutral | P168I | 2258 | 560.15 | 568.90 | 0.98 | 0.09 | 0.08 |
| Neutral | P168D | 2243 | 530.69 | 575.63 | 0.92 | 0.09 | 0.08 |
| Neutral | G169H | 2264 | 791.08 | 828.63 | 0.95 | 0.13 | 0.12 |
| Down | G169A | 2279 | 1556.29 | 2632.37 | 0.59 | 0.26 | 0.37 |
| Neutral | G169E | 2263 | 789.82 | 829.24 | 0.95 | 0.13 | 0.12 |
| Neutral | G169C | 2267 | 714.55 | 744.33 | 0.96 | 0.12 | 0.10 |
| Neutral | G169S | 2272 | 1196.93 | 1427.18 | 0.84 | 0.20 | 0.20 |
| Neutral | G169L | 2278 | 450.44 | 534.57 | 0.84 | 0.08 | 0.07 |
| Neutral | G169V | 2277 | 703.56 | 675.20 | 1.04 | 0.12 | 0.09 |
| Neutral | G169T | 2270 | 676.59 | 685.16 | 0.99 | 0.11 | 0.10 |
| Neutral | G169R | 2266 | 1119.16 | 1166.00 | 0.96 | 0.19 | 0.16 |
| Neutral | G169W | 2275 | 802.02 | 921.44 | 0.87 | 0.14 | 0.13 |
| Neutral | G169M | 2274 | 962.20 | 1133.87 | 0.85 | 0.16 | 0.16 |
| Neutral | G169I | 2276 | 671.79 | 677.10 | 0.99 | 0.11 | 0.09 |
| Neutral | G169P | 2280 | 671.60 | 683.22 | 0.98 | 0.11 | 0.10 |
| Neutral | G169D | 2262 | 714.59 | 766.96 | 0.93 | 0.12 | 0.11 |
| Neutral | G169Q | 2269 | 977.05 | 901.01 | 1.08 | 0.17 | 0.13 |
| Down | P170L | 2298 | 5969.84 | 7995.99 | 0.75 | 1.01 | 1.12 |
| Down | P17OR | 2285 | 3566.07 | 5876.72 | 0.61 | 0.60 | 0.82 |
| Down | P1701 | 2296 | 5073.27 | 7150.78 | 0.71 | 0.86 | 1.00 |
| Neutral | P170T | 2289 | 6734.46 | 8153.81 | 0.83 | 1.14 | 1.14 |
| Down | P170F | 2293 | 2114.36 | 3365.04 | 0.63 | 0.36 | 0.47 |
| Down | P170Q | 2288 | 4204.94 | 6162.63 | 0.68 | 0.71 | 0.86 |
| Down | P170G | 2292 | 5005.05 | 6924.03 | 0.72 | 0.85 | 0.97 |
| Down | P170S | 2291 | 4526.99 | 6064.79 | 0.75 | 0.77 | 0.85 |
| Down | P170H | 2283 | 4569.14 | 6879.10 | 0.66 | 0.77 | 0.96 |
| Down | P170C | 2286 | 931.84 | 2355.40 | 0.40 | 0.16 | 0.33 |
| Down | P170M | 2294 | 3323.56 | 6318.16 | 0.53 | 0.56 | 0.89 |
| Down | P170K | 2284 | 4379.75 | 6206.45 | 0.71 | 0.74 | 0.87 |
| Down | P170W | 2295 | 1794.33 | 2781.05 | 0.65 | 0.30 | 0.39 |
| Neutral | P170D | 2281 | 1434.38 | 1462.91 | 0.98 | 0.25 | 0.29 |
| Neutral | P170A | 2299 | 2733.72 | 2793.24 | 0.98 | 0.48 | 0.55 |
| Down | G171S | 564 | 2129.39 | 3316.87 | 0.64 | 0.36 | 0.46 |
| Down | G171M | 566 | 2104.33 | 3308.36 | 0.64 | 0.36 | 0.46 |
| Down | G171N | 560 | 4674.81 | 6965.17 | 0.67 | 0.79 | 0.98 |
| Up | G171P | 572 | 1570.74 | 1204.39 | 1.30 | 0.27 | 0.17 |
| Down | G171R | 558 | 1604.06 | 2486.74 | 0.65 | 0.27 | 0.35 |
| Down | G171Y | 563 | 1519.56 | 2342.05 | 0.65 | 0.26 | 0.33 |
| Down | G171A | 571 | 3517.89 | 5269.99 | 0.67 | 0.60 | 0.74 |
| Down | G171Q | 561 | 2361.29 | 3915.33 | 0.60 | 0.40 | 0.55 |
| Down | G171H | 556 | 1662.65 | 2616.63 | 0.64 | 0.28 | 0.37 |
| Down | G171L | 570 | 1551.95 | 2516.17 | 0.62 | 0.26 | 0.35 |
| Down | G171W | 567 | 1068.10 | 1663.46 | 0.64 | 0.18 | 0.23 |
| Down | G171C | 559 | 1982.45 | 3409.68 | 0.58 | 0.34 | 0.48 |
| Down | G171K | 557 | 1324.98 | 1867.15 | 0.71 | 0.22 | 0.26 |
| Neutral | G171E | 555 | 1154.96 | 1199.65 | 0.96 | 0.20 | 0.24 |
| Neutral | G171D | 554 | 791.81 | 690.33 | 1.15 | 0.14 | 0.14 |
| Neutral | I172Y | 2309 | 7427.25 | 7893.50 | 0.94 | 0.93 | 0.95 |
| Neutral | I172T | 2308 | 5861.67 | 6776.91 | 0.86 | 0.73 | 0.81 |
| Neutral | I172P | 2318 | 6297.28 | 7073.49 | 0.89 | 0.79 | 0.85 |
| Down | I172A | 2317 | 4666.76 | 6048.64 | 0.77 | 0.58 | 0.73 |
| Neutral | I172L | 2316 | 9324.48 | 8876.38 | 1.05 | 1.17 | 1.07 |
| Neutral | I172Q | 2307 | 6906.03 | 7743.66 | 0.89 | 0.87 | 0.93 |
| Down | I172E | 2301 | 3517.33 | 4567.20 | 0.77 | 0.44 | 0.55 |
| Down | I172C | 2305 | 1784.43 | 2422.99 | 0.74 | 0.22 | 0.29 |
| Neutral | I172M | 2313 | 9859.60 | 9096.03 | 1.08 | 1.24 | 1.09 |
| Neutral | I172D | 2300 | 4276.25 | 4281.60 | 1.00 | 0.54 | 0.51 |
| Neutral | I172V | 2315 | 9541.02 | 9174.91 | 1.04 | 1.20 | 1.10 |
| Neutral | I172R | 2304 | 7010.86 | 7581.48 | 0.92 | 0.88 | 0.91 |
| Neutral | I172G | 2311 | 2169.59 | 2350.53 | 0.92 | 0.27 | 0.28 |
| Neutral | I172W | 2314 | 5870.25 | 7244.46 | 0.81 | 0.74 | 0.87 |
| Neutral | I172N | 2306 | 5235.67 | 6253.19 | 0.84 | 0.66 | 0.75 |
| Down | G173C | 2324 | 816.12 | 1115.07 | 0.73 | 0.10 | 0.13 |
| Neutral | G173L | 2325 | 454.21 | 401.73 | 1.13 | 0.06 | 0.05 |
| Neutral | G173K | 2322 | 741.76 | 685.79 | 1.08 | 0.09 | 0.08 |
| Neutral | G173W | 2332 | 1278.44 | 1132.78 | 1.13 | 0.16 | 0.14 |
| Neutral | G173S | 2329 | 865.76 | 793.10 | 1.09 | 0.11 | 0.10 |
| Neutral | G173A | 2326 | 1041.22 | 1038.66 | 1.00 | 0.13 | 0.12 |
| Neutral | G173R | 2323 | 973.70 | 877.71 | 1.11 | 0.12 | 0.11 |
| Neutral | G173N | 2325 | 818.38 | 931.59 | 0.88 | 0.10 | 0.11 |
| Neutral | G173T | 2327 | 481.41 | 485.39 | 0.99 | 0.06 | 0.06 |
| Neutral | G173D | 2319 | 474.45 | 424.47 | 1.12 | 0.06 | 0.05 |
| Neutral | G173V | 2324 | 505.39 | 476.04 | 1.06 | 0.06 | 0.06 |
| Neutral | G173F | 2330 | 670.10 | 610.91 | 1.10 | 0.08 | 0.07 |
| Neutral | G173M | 2331 | 1085.74 | 1324.87 | 0.82 | 0.14 | 0.16 |
| Neutral | G173Y | 2328 | 1390.14 | 1296.93 | 1.07 | 0.17 | 0.16 |
| Neutral | G173P | 2327 | 458.79 | 435.59 | 1.05 | 0.06 | 0.05 |
| Neutral | G174R | 2342 | 452.76 | 437.41 | 1.04 | 0.06 | 0.05 |
| Neutral | G174A | 2355 | 491.35 | 460.84 | 1.07 | 0.06 | 0.06 |
| Neutral | G174E | 2339 | 489.49 | 459.57 | 1.07 | 0.06 | 0.06 |
| Neutral | G174F | 2349 | 598.90 | 520.03 | 1.15 | 0.08 | 0.06 |
| Neutral | G174H | 2340 | 577.19 | 518.90 | 1.11 | 0.07 | 0.06 |
| Neutral | G174T | 2346 | 505.38 | 483.64 | 1.04 | 0.06 | 0.06 |
| Neutral | G174D | 2338 | 476.79 | 454.13 | 1.05 | 0.06 | 0.05 |
| Neutral | G174S | 2348 | 641.62 | 580.36 | 1.11 | 0.08 | 0.07 |
| Neutral | G174P | 2356 | 525.07 | 505.41 | 1.04 | 0.07 | 0.06 |
| Neutral | G174W | 2351 | 538.38 | 502.50 | 1.07 | 0.07 | 0.06 |
| Neutral | G174V | 2353 | 504.50 | 425.17 | 1.19 | 0.06 | 0.05 |
| Neutral | G174N | 2344 | 506.54 | 460.95 | 1.10 | 0.06 | 0.06 |
| Up | G174Y | 2347 | 722.10 | 525.97 | 1.37 | 0.09 | 0.06 |
| Neutral | G174M | 2350 | 516.58 | 464.32 | 1.11 | 0.06 | 0.06 |
| Neutral | G174L | 2354 | 474.83 | 436.08 | 1.09 | 0.06 | 0.05 |
| Neutral | D1751 | 2371 | 697.29 | 708.65 | 0.98 | 0.09 | 0.09 |
| Neutral | D175T | 2364 | 601.82 | 560.11 | 1.07 | 0.08 | 0.07 |
| Neutral | D175N | 2362 | 1495.98 | 1413.58 | 1.06 | 0.19 | 0.17 |
| Neutral | D175V | 2372 | 694.28 | 652.82 | 1.06 | 0.09 | 0.08 |
| Neutral | D175S | 2366 | 664.04 | 579.78 | 1.15 | 0.08 | 0.07 |
| Neutral | D175R | 2360 | 593.04 | 505.98 | 1.17 | 0.07 | 0.06 |
| Down | D175G | 2367 | 3147.62 | 4207.02 | 0.75 | 0.39 | 0.51 |
| Up | D175A | 2374 | 633.65 | 519.61 | 1.22 | 0.08 | 0.06 |
| Neutral | D175F | 2368 | 768.41 | 804.63 | 0.95 | 0.10 | 0.10 |
| Neutral | D175C | 2361 | 535.75 | 498.44 | 1.07 | 0.07 | 0.06 |
| Neutral | D175Q | 2363 | 702.74 | 633.64 | 1.11 | 0.09 | 0.08 |
| Neutral | D175Y | 2365 | 574.71 | 539.79 | 1.06 | 0.07 | 0.06 |
| Neutral | D175L | 2373 | 591.05 | 549.49 | 1.08 | 0.07 | 0.07 |
| Up | D175H | 2358 | 830.09 | 564.37 | 1.47 | 0.10 | 0.07 |
| Neutral | D175P | 2375 | 635.08 | 610.89 | 1.04 | 0.08 | 0.07 |
| Up | D175E | 2357 | 959.14 | 495.19 | 1.94 | 0.10 | 0.07 |
| Up | A176F | 148 | 10486.82 | 6516.31 | 1.61 | 1.31 | 0.78 |
| Neutral | A176Q | 143 | 6410.52 | 6665.27 | 0.96 | 0.80 | 0.80 |
| Neutral | A176V | 152 | 8890.53 | 8780.42 | 1.01 | 1.11 | 1.05 |
| Neutral | A176E | 137 | 589.82 | 546.54 | 1.08 | 0.07 | 0.07 |
| Neutral | A176T | 144 | 8471.98 | 8213.74 | 1.03 | 1.06 | 0.99 |
| Neutral | A176C | 141 | 6777.92 | 5924.96 | 1.14 | 0.85 | 0.71 |
| Neutral | A176L | 153 | 7190.01 | 6291.31 | 1.14 | 0.90 | 0.76 |
| Neutral | A176P | 154 | 639.90 | 596.40 | 1.07 | 0.08 | 0.07 |
| Neutral | A176N | 142 | 1351.92 | 1250.94 | 1.08 | 0.17 | 0.15 |
| Neutral | A176G | 147 | 2185.25 | 2395.33 | 0.91 | 0.27 | 0.29 |
| Down | A176S | 146 | 3003.29 | 3887.12 | 0.77 | 0.38 | 0.47 |
| Neutral | A176R | 140 | 919.15 | 792.44 | 1.16 | 0.12 | 0.10 |
| Neutral | A176K | 139 | 561.66 | 522.94 | 1.07 | 0.07 | 0.06 |
| Neutral | A176D | 136 | 863.82 | 792.98 | 1.09 | 0.11 | 0.10 |
| Neutral | A176W | 150 | 482.38 | 414.85 | 1.16 | 0.06 | 0.06 |
| Neutral | H177T | 2383 | 600.03 | 570.23 | 1.05 | 0.08 | 0.07 |
| Neutral | H177P | 2394 | 579.96 | 544.77 | 1.06 | 0.07 | 0.07 |
| Neutral | H177Q | 2382 | 593.68 | 549.35 | 1.08 | 0.07 | 0.07 |
| Neutral | H177A | 2393 | 536.01 | 523.32 | 1.02 | 0.07 | 0.06 |
| Neutral | H177S | 2385 | 561.60 | 524.64 | 1.07 | 0.07 | 0.06 |
| Neutral | H177G | 2386 | 559.31 | 519.85 | 1.08 | 0.07 | 0.06 |
| Neutral | H177W | 2389 | 547.21 | 520.18 | 1.05 | 0.07 | 0.06 |
| Neutral | H177L | 2392 | 486.50 | 433.60 | 1.12 | 0.06 | 0.05 |
| Neutral | H177V | 2391 | 508.58 | 447.50 | 1.14 | 0.06 | 0.05 |
| Neutral | H1771 | 2390 | 489.45 | 455.90 | 1.07 | 0.06 | 0.05 |
| Down | H177R | 2379 | 1913.95 | 2460.77 | 0.78 | 0.24 | 0.30 |
| Neutral | H177N | 2381 | 504.44 | 478.07 | 1.06 | 0.06 | 0.06 |
| Neutral | H177Y | 2384 | 519.99 | 467.72 | 1.11 | 0.07 | 0.06 |
| Neutral | H177C | 2380 | 521.67 | 489.93 | 1.06 | 0.07 | 0.06 |
| Neutral | H177D | 2376 | 534.87 | 505.07 | 1.06 | 0.07 | 0.06 |
| Neutral | F178G | 2406 | 451.96 | 391.96 | 1.15 | 0.05 | 0.05 |
| Up | F178C | 2400 | 491.76 | 403.57 | 1.22 | 0.05 | 0.05 |
| Neutral | F178W | 2408 | 488.21 | 441.35 | 1.11 | 0.05 | 0.05 |
| Neutral | F178R | 2399 | 492.40 | 411.21 | 1.20 | 0.05 | 0.05 |
| Neutral | F178K | 2398 | 490.87 | 494.09 | 0.99 | 0.05 | 0.06 |
| Neutral | F178S | 2405 | 489.84 | 507.26 | 0.97 | 0.05 | 0.06 |
| Neutral | F178H | 2397 | 525.63 | 500.02 | 1.05 | 0.06 | 0.06 |
| Neutral | F178P | 2413 | 441.78 | 397.05 | 1.11 | 0.05 | 0.05 |
| Neutral | F178V | 2410 | 742.61 | 814.06 | 0.91 | 0.08 | 0.10 |
| Neutral | F178A | 2412 | 421.25 | 367.26 | 1.15 | 0.05 | 0.04 |
| Neutral | F178Q | 2402 | 409.62 | 360.29 | 1.14 | 0.04 | 0.04 |
| Neutral | F178Y | 2404 | 861.20 | 830.80 | 1.04 | 0.09 | 0.10 |
| Neutral | F178I | 2409 | 1118.23 | 1329.96 | 0.84 | 0.12 | 0.16 |
| Neutral | F178T | 2403 | 560.54 | 487.01 | 1.15 | 0.10 | 0.10 |
| Up | F178L | 2411 | 1788.95 | 1314.38 | 1.36 | 0.31 | 0.26 |
| Neutral | F178E | 2396 | 524.72 | 515.62 | 1.02 | 0.06 | 0.07 |
| Neutral | D179P | 173 | 526.54 | 527.98 | 1.00 | 0.06 | 0.06 |
| Neutral | D179L | 171 | 444.31 | 410.22 | 1.08 | 0.05 | 0.05 |
| Neutral | D179E | 155 | 520.82 | 438.27 | 1.19 | 0.06 | 0.05 |
| Neutral | D179G | 165 | 470.21 | 426.64 | 1.10 | 0.05 | 0.05 |
| Neutral | D179S | 164 | 461.51 | 421.09 | 1.10 | 0.05 | 0.05 |
| Neutral | D179A | 172 | 464.49 | 431.24 | 1.08 | 0.05 | 0.05 |
| Neutral | D179K | 157 | 483.67 | 456.75 | 1.06 | 0.05 | 0.05 |
| Neutral | D179T | 162 | 451.18 | 419.76 | 1.07 | 0.05 | 0.05 |
| Neutral | D179I | 169 | 425.91 | 372.56 | 1.14 | 0.05 | 0.04 |
| Neutral | D179R | 158 | 473.21 | 450.10 | 1.05 | 0.05 | 0.05 |
| Up | D179N | 160 | 2433.73 | 812.01 | 3.00 | 0.26 | 0.10 |
| Neutral | D179W | 168 | 465.31 | 423.56 | 1.10 | 0.05 | 0.05 |
| Neutral | D179Q | 161 | 446.51 | 414.79 | 1.08 | 0.05 | 0.05 |
| Up | D179V | 170 | 604.63 | 490.35 | 1.23 | 0.11 | 0.10 |
| Up | D179C | 159 | 613.81 | 503.76 | 1.22 | 0.11 | 0.10 |
| Neutral | E180M | 186 | 9630.23 | 8513.41 | 1.13 | 1.04 | 1.00 |
| Neutral | E180P | 192 | 523.92 | 492.75 | 1.06 | 0.06 | 0.06 |
| Neutral | E180K | 176 | 4017.43 | 3889.45 | 1.03 | 0.43 | 0.46 |
| Up | E180Y | 182 | 6655.19 | 5379.42 | 1.24 | 0.72 | 0.63 |
| Neutral | E180Q | 180 | 5146.93 | 4568.90 | 1.13 | 0.56 | 0.54 |
| Neutral | E180R | 177 | 6932.51 | 6309.81 | 1.10 | 0.75 | 0.74 |
| Neutral | E180A | 191 | 9562.37 | 8450.18 | 1.13 | 1.04 | 1.00 |
| Up | E180T | 181 | 3718.16 | 2425.13 | 1.53 | 0.40 | 0.29 |
| Neutral | E1801 | 188 | 9126.95 | 7770.14 | 1.17 | 0.99 | 0.92 |
| Up | E180F | 185 | 7014.78 | 5382.78 | 1.30 | 0.76 | 0.63 |
| Neutral | E180C | 178 | 2926.15 | 2569.75 | 1.14 | 0.32 | 0.30 |
| Up | E180G | 184 | 5952.65 | 4547.28 | 1.31 | 1.04 | 0.90 |
| Up | E180S | 183 | 5217.80 | 3977.60 | 1.31 | 0.91 | 0.78 |
| Up | E180N | 179 | 6534.65 | 4843.84 | 1.35 | 1.14 | 0.96 |
| Up | E180D | 174 | 7738.70 | 6277.22 | 1.23 | 1.35 | 1.24 |
| Neutral | D181S | 202 | 9064.64 | 8368.97 | 1.08 | 0.98 | 0.99 |
| Neutral | D181Q | 199 | 7875.57 | 7127.19 | 1.11 | 0.85 | 0.84 |
| Neutral | D181P | 211 | 753.20 | 639.76 | 1.18 | 0.08 | 0.08 |
| Up | D181Y | 201 | 1137.94 | 716.86 | 1.59 | 0.12 | 0.08 |
| Up | D181R | 196 | 997.11 | 712.77 | 1.40 | 0.11 | 0.08 |
| Up | D181V | 208 | 945.65 | 721.77 | 1.31 | 0.10 | 0.09 |
| UP | D181F | 204 | 933.48 | 670.40 | 1.39 | 0.10 | 0.08 |
| Neutral | D181A | 210 | 7936.89 | 7854.96 | 1.01 | 0.86 | 0.93 |
| Neutral | D181T | 200 | 6867.00 | 6057.09 | 1.13 | 0.74 | 0.71 |
| Up | D181L | 209 | 1727.20 | 1274.09 | 1.36 | 0.19 | 0.15 |
| Neutral | D181E | 193 | 8647.28 | 8246.36 | 1.05 | 0.94 | 0.97 |
| Up | D181K | 195 | 1087.36 | 696.83 | 1.56 | 0.12 | 0.08 |
| Up | D181M | 205 | 3805.65 | 2986.75 | 1.27 | 0.41 | 0.35 |
| Up | D181C | 197 | 549.29 | 447.40 | 1.23 | 0.10 | 0.09 |
| Up | D181G | 203 | 2764.20 | 2056.56 | 1.34 | 0.48 | 0.41 |
| Neutral | E182C | 216 | 601.45 | 561.21 | 1.07 | 0.07 | 0.07 |
| Neutral | E182P | 230 | 606.01 | 574.24 | 1.06 | 0.07 | 0.07 |
| Up | E182S | 221 | 967.49 | 642.27 | 1.51 | 0.10 | 0.08 |
| Up | E182T | 219 | 2995.97 | 1779.42 | 1.68 | 0.32 | 0.21 |
| Neutral | E182R | 215 | 661.10 | 622.75 | 1.06 | 0.07 | 0.07 |
| Neutral | E182D | 212 | 2078.47 | 2140.28 | 0.97 | 0.23 | 0.25 |
| Neutral | E182A | 229 | 619.23 | 531.55 | 1.16 | 0.07 | 0.06 |
| Neutral | E182F | 223 | 1484.85 | 1677.82 | 0.88 | 0.16 | 0.20 |
| Neutral | E182L | 228 | 569.35 | 524.25 | 1.09 | 0.06 | 0.06 |
| Neutral | E182I | 226 | 606.88 | 519.75 | 1.17 | 0.07 | 0.06 |
| Neutral | E182Y | 220 | 593.61 | 561.88 | 1.06 | 0.06 | 0.07 |
| Up | E182Q | 218 | 1393.28 | 804.84 | 1.73 | 0.15 | 0.09 |
| Neutral | E182W | 225 | 556.78 | 536.32 | 1.04 | 0.06 | 0.06 |
| Up | E182M | 224 | 649.73 | 524.43 | 1.24 | 0.11 | 0.10 |
| Neutral | E182G | 222 | 604.92 | 543.78 | 1.11 | 0.11 | 0.11 |
| Neutral | R183P | 2432 | 9143.00 | 8148.29 | 1.12 | 0.99 | 0.96 |
| Neutral | R183K | 2417 | 9843.98 | 8685.25 | 1.13 | 1.07 | 1.02 |
| Neutral | R183W | 2427 | 8144.07 | 7669.02 | 1.06 | 0.88 | 0.90 |
| Neutral | R183E | 2415 | 9873.25 | 8403.44 | 1.17 | 1.07 | 0.99 |
| Neutral | R183A | 2431 | 9386.14 | 8368.29 | 1.12 | 1.02 | 0.99 |
| Down | R183T | 2421 | 4841.94 | 8385.09 | 0.58 | 0.52 | 0.99 |
| Neutral | R183L | 2430 | 517.07 | 532.72 | 0.97 | 0.06 | 0.06 |
| Neutral | R183N | 2419 | 10062.02 | 8456.13 | 1.19 | 1.09 | 1.00 |
| Neutral | R183H | 2416 | 9434.01 | 8295.55 | 1.14 | 1.02 | 0.98 |
| Neutral | R183V | 2429 | 9252.08 | 7954.42 | 1.16 | 1.00 | 0.94 |
| Neutral | R183C | 2418 | 6603.93 | 6597.30 | 1.00 | 0.72 | 0.78 |
| Neutral | R183M | 2426 | 9679.52 | 8250.27 | 1.17 | 1.05 | 0.97 |
| Down | R183I | 2428 | 495.34 | 8009.63 | 0.06 | 0.05 | 0.94 |
| Up | R183G | 2424 | 7326.36 | 6021.39 | 1.22 | 1.28 | 1.19 |
| Up | R183S | 2423 | 7896.17 | 6240.74 | 1.27 | 1.38 | 1.23 |
| Neutral | W184G | 2444 | 430.62 | 391.79 | 1.10 | 0.05 | 0.05 |
| Neutral | W184H | 2435 | 440.24 | 428.35 | 1.03 | 0.05 | 0.05 |
| Neutral | W184L | 2449 | 476.77 | 428.90 | 1.11 | 0.06 | 0.05 |
| Neutral | W184E | 2434 | 463.88 | 438.97 | 1.06 | 0.05 | 0.05 |
| Neutral | W184P | 2451 | 437.57 | 387.71 | 1.13 | 0.05 | 0.05 |
| Neutral | W184N | 2439 | 467.91 | 468.52 | 1.00 | 0.06 | 0.06 |
| Neutral | W184A | 2450 | 452.63 | 451.66 | 1.00 | 0.05 | 0.06 |
| Neutral | W184T | 2441 | 421.08 | 419.51 | 1.00 | 0.05 | 0.05 |
| Neutral | W184R | 2437 | 457.42 | 390.02 | 1.17 | 0.05 | 0.05 |
| Neutral | W184Q | 2440 | 450.92 | 448.33 | 1.01 | 0.05 | 0.05 |
| Neutral | W184V | 2448 | 454.60 | 407.30 | 1.12 | 0.05 | 0.05 |
| Neutral | W184S | 2443 | 486.70 | 485.16 | 1.00 | 0.06 | 0.06 |
| Neutral | W184M | 2446 | 447.30 | 395.61 | 1.13 | 0.05 | 0.05 |
| Neutral | W184I | 2447 | 478.17 | 503.24 | 0.95 | 0.06 | 0.06 |
| Neutral | W184F | 2445 | 455.86 | 427.51 | 1.07 | 0.05 | 0.05 |
| Up | T185R | 235 | 1728.04 | 851.07 | 2.03 | 0.20 | 0.10 |
| Up | T185Y | 239 | 937.75 | 540.66 | 1.73 | 0.11 | 0.07 |
| Neutral | T185W | 244 | 577.54 | 501.10 | 1.15 | 0.07 | 0.06 |
| Up | T185H | 233 | 1448.04 | 783.89 | 1.85 | 0.17 | 0.10 |
| Up | T185G | 241 | 3922.30 | 1990.15 | 1.97 | 0.46 | 0.24 |
| Neutral | T185P | 249 | 1773.27 | 1542.44 | 1.15 | 0.21 | 0.19 |
| Neutral | T185S | 240 | 9554.77 | 8267.62 | 1.16 | 1.12 | 1.01 |
| Up | T185V | 246 | 1648.14 | 897.66 | 1.84 | 0.19 | 0.11 |
| Up | T185Q | 238 | 1594.81 | 583.93 | 2.73 | 0.19 | 0.07 |
| Up | T185N | 237 | 790.61 | 546.44 | 1.45 | 0.09 | 0.07 |
| Up | T185C | 236 | 1554.42 | 1248.58 | 1.24 | 0.18 | 0.15 |
| Neutral | T185L | 247 | 483.25 | 463.52 | 1.04 | 0.06 | 0.06 |
| Up | T185A | 248 | 1599.64 | 711.08 | 2.25 | 0.19 | 0.09 |
| Up | TI85E | 232 | 1324.02 | 703.76 | 1.88 | 0.16 | 0.09 |
| Neutral | T185D | 231 | 485.86 | 418.67 | 1.16 | 0.06 | 0.06 |
| Neutral | N186G | 2462 | 7592.31 | 6944.43 | 1.09 | 0.89 | 0.85 |
| Neutral | N186A | 2469 | 7466.07 | 7519.13 | 0.99 | 0.88 | 0.92 |
| Neutral | N186T | 2459 | 8897.05 | 8063.02 | 1.10 | 1.05 | 0.98 |
| Neutral | N186R | 2456 | 3212.69 | 3085.21 | 1.04 | 0.38 | 0.38 |
| Neutral | N186L | 2468 | 8097.42 | 7286.32 | 1.11 | 0.95 | 0.89 |
| Neutral | N186P | 2470 | 2173.37 | 1948.86 | 1.12 | 0.26 | 0.24 |
| Neutral | N186S | 2461 | 6854.56 | 6735.79 | 1.02 | 0.81 | 0.82 |
| Neutral | N186V | 2467 | 6303.91 | 6575.96 | 0.96 | 0.74 | 0.80 |
| Neutral | N186Q | 2458 | 4834.56 | 4621.18 | 1.05 | 0.57 | 0.56 |
| Neutral | NI86H | 2454 | 3390.53 | 3309.97 | 1.02 | 0.40 | 0.40 |
| Neutral | N186C | 2457 | 3139.47 | 3113.35 | 1.01 | 0.37 | 0.38 |
| Neutral | N186E | 2453 | 3801.36 | 3332.52 | 1.14 | 0.45 | 0.41 |
| Neutral | N186F | 2463 | 3794.65 | 3316.48 | 1.14 | 0.45 | 0.40 |
| Neutral | N186Y | 2460 | 6301.09 | 7570.59 | 0.83 | 0.74 | 0.92 |
| Neutral | N186D | 2452 | 6853.09 | 6333.37 | 1.08 | 0.81 | 0.77 |
| Up | N187R | 254 | 1042.36 | 709.74 | 1.47 | 0.12 | 0.09 |
| Up | N187M | 262 | 1731.67 | 995.07 | 1.74 | 0.20 | 0.12 |
| Neutral | N187S | 259 | 9538.59 | 8971.12 | 1.06 | 1.12 | 1.10 |
| Neutral | N187T | 257 | 9856.38 | 8855.58 | 1.11 | 1.16 | 1.08 |
| Neutral | N187L | 266 | 505.93 | 464.62 | 1.09 | 0.06 | 0.06 |
| Neutral | N187W | 263 | 1694.86 | 1425.68 | 1.19 | 0.20 | 0.17 |
| Up | N187F | 261 | 1240.41 | 731.98 | 1.69 | 0.15 | 0.09 |
| Up | N187K | 253 | 2331.93 | 1140.19 | 2.05 | 0.27 | 0.14 |
| Up | N187I | 264 | 1444.98 | 683.03 | 2.12 | 0.17 | 0.08 |
| Up | N187A | 267 | 4379.80 | 2616.49 | 1.67 | 0.52 | 0.32 |
| Neutral | N187P | 268 | 644.27 | 572.98 | 1.12 | 0.08 | 0.07 |
| Neutral | N187D | 250 | 9843.65 | 8801.57 | 1.12 | 1.16 | 1.07 |
| Neutral | N187G | 260 | 535.06 | 514.10 | 1.04 | 0.07 | 0.07 |
| Neutral | N187C | 255 | 1804.28 | 1860.67 | 0.97 | 0.23 | 0.25 |
| Neutral | N187H | 252 | 1143.07 | 1071.67 | 1.07 | 0.14 | 0.14 |
| Neutral | F188P | 2489 | 10012.21 | 8943.91 | 1.12 | 1.18 | 1.09 |
| Neutral | F188I | 2485 | 7342.21 | 6782.40 | 1.08 | 0.86 | 0.83 |
| Neutral | F188N | 2477 | 10024.22 | 8961.63 | 1.12 | 1.18 | 1.09 |
| Neutral | F188S | 2481 | 9564.51 | 8841.98 | 1.08 | 1.13 | 1.08 |
| Neutral | F188Q | 2478 | 9591.39 | 8664.63 | 1.11 | 1.13 | 1.06 |
| Neutral | P188K | 2474 | 8347.12 | 7497.38 | 1.11 | 0.98 | 0.92 |
| Neutral | F188G | 2482 | 9891.61 | 9065.43 | 1.09 | 1.16 | 1.11 |
| Neutral | F188W | 2484 | 9389.97 | 8774.36 | 1.07 | 1.10 | 1.07 |
| Neutral | F188E | 2472 | 10235.38 | 8984.46 | 1.14 | 1.20 | 1.10 |
| Neutral | F188H | 2473 | 2065.12 | 1901.41 | 1.09 | 0.24 | 0.23 |
| Neutral | F188D | 2471 | 10087.61 | 8889.75 | 1.13 | 1.19 | 1.09 |
| Up | F188A | 2488 | 1502.70 | 1231.99 | 1.22 | 0.18 | 0.15 |
| Neutral | F188L | 2487 | 8309.64 | 7501.30 | 1.11 | 0.98 | 0.92 |
| Neutral | F188R | 2475 | 8182.64 | 7750.05 | 1.06 | 0.96 | 0.95 |
| Up | F188V | 2486 | 7116.29 | 5860.00 | 1.21 | 1.24 | 1.16 |
| Neutral | R189L | 2506 | 9236.08 | 8947.54 | 1.03 | 1.09 | 1.09 |
| Neutral | R189G | 2500 | 10307.88 | 9096.35 | 1.13 | 1.21 | 1.11 |
| Neutral | R189K | 2493 | 9365.15 | 9033.15 | 1.04 | 1.10 | 1.10 |
| Neutral | R189P | 2508 | 3200.68 | 3533.96 | 0.91 | 0.38 | 0.43 |
| Neutral | R189E | 2491 | 9552.57 | 8789.28 | 1.09 | 1.12 | 1.07 |
| Neutral | R189V | 2505 | 9150.17 | 8088.39 | 1.13 | 1.08 | 0.99 |
| Neutral | R189D | 2490 | 9506.16 | 8933.41 | 1.06 | 1.12 | 1.09 |
| Neutral | R189Y | 2498 | 9893.14 | 8946.52 | 1.11 | 1.16 | 1.09 |
| Neutral | R189C | 2494 | 5318.06 | 5457.78 | 0.97 | 0.63 | 0.67 |
| Neutral | R189A | 2507 | 9718.09 | 8798.13 | 1.10 | 1.14 | 1.07 |
| Neutral | R189H | 2492 | 1360.90 | 1350.33 | 1.01 | 0.16 | 0.16 |
| Neutral | R189W | 2503 | 7657.12 | 7070.92 | 1.08 | 0.90 | 0.86 |
| Neutral | R189N | 2495 | 7842.39 | 6675.36 | 1.17 | 1.37 | 1.32 |
| Neutral | R189T | 2497 | 7610.10 | 6459.94 | 1.18 | 1.33 | 1.27 |
| Neutral | R189Q | 2496 | 7465.37 | 6396.79 | 1.17 | 1.30 | 1.26 |
| Down | E190A | 590 | 1510.06 | 2116.94 | 0.71 | 0.21 | 0.23 |
| Neutral | E190H | 574 | 6276.13 | 7564.04 | 0.83 | 0.88 | 0.83 |
| Down | E190V | 588 | 643.37 | 1658.11 | 0.39 | 0.09 | 0.18 |
| Up | E190P | 591 | 2420.68 | 1767.43 | 1.37 | 0.34 | 0.19 |
| Neutral | E190C | 577 | 1827.25 | 2083.88 | 0.88 | 0.26 | 0.23 |
| Up | E190G | 583 | 5313.99 | 4365.93 | 1.22 | 0.75 | 0.48 |
| Down | E190R | 576 | 1185.26 | 1810.53 | 0.65 | 0.17 | 0.20 |
| Down | E1901 | 587 | 1880.80 | 2886.28 | 0.65 | 0.27 | 0.32 |
| Down | E190S | 582 | 4542.61 | 5987.33 | 0.76 | 0.64 | 0.66 |
| Down | E190T | 580 | 2293.47 | 4444.68 | 0.52 | 0.32 | 0.49 |
| Up | E190M | 585 | 2557.21 | 1317.73 | 1.94 | 0.36 | 0.15 |
| Neutral | E190L | 589 | 2542.38 | 2986.91 | 0.85 | 0.36 | 0.33 |
| Down | E190K | 575 | 2960.37 | 4343.12 | 0.68 | 0.42 | 0.48 |
| Up | E190Y | 581 | 7243.54 | 5742.33 | 1.26 | 1.27 | 1.13 |
| Up | E190D | 573 | 7910.21 | 6468.78 | 1.22 | 1.38 | 1.28 |
| Neutral | Y191T | 600 | 611.75 | 535.95 | 1.14 | 0.07 | 0.06 |
| Neutral | Y191H | 594 | 2333.85 | 2191.64 | 1.06 | 0.28 | 0.24 |
| Neutral | Y191G | 602 | 428.17 | 432.65 | 0.99 | 0.05 | 0.05 |
| Neutral | Y191L | 608 | 379.02 | 357.82 | 1.06 | 0.05 | 0.04 |
| Up | Y191P | 610 | 1359.30 | 1046.33 | 1.30 | 0.16 | 0.12 |
| Neutral | Y191Q | 599 | 451.92 | 403.46 | 1.12 | 0.05 | 0.05 |
| Neutral | Y191K | 595 | 464.62 | 464.52 | 1.14 | 0.06 | 0.05 |
| Neutral | Y191D | 592 | 392.24 | 370.67 | 1.06 | 0.05 | 0.04 |
| Neutral | Y191A | 609 | 452.13 | 418.53 | 1.08 | 0.05 | 0.05 |
| Neutral | Y191W | 605 | 395.63 | 411.91 | 0.96 | 0.05 | 0.05 |
| Neutral | Y191S | 601 | 530.80 | 447.13 | 1.19 | 0.06 | 0.05 |
| Up | Y191V | 607 | 1553.58 | 1254.11 | 1.24 | 0.19 | 0.14 |
| Neutral | Y191E | 593 | 395.04 | 407.49 | 0.97 | 0.05 | 0.05 |
| Neutral | Y191R | 596 | 652.95 | 725.68 | 0.90 | 0.08 | 0.08 |
| Neutral | Y191C | 597 | 530.42 | 463.90 | 1.14 | 0.06 | 0.05 |
| Up | N192R | 615 | 640.72 | 482.61 | 1.33 | 0.09 | 0.05 |
| Neutral | N192L | 627 | 591.92 | 571.56 | 1.04 | 0.08 | 0.06 |
| Neutral | N192Q | 617 | 1089.41 | 1020.23 | 1.07 | 0.15 | 0.11 |
| Neutral | N192P | 629 | 685.62 | 856.11 | 0.80 | 0.10 | 0.09 |
| Up | N192H | 613 | 2274.24 | 1058.80 | 2.15 | 0.32 | 0.12 |
| Up | N192S | 620 | 2043.65 | 1630.74 | 1.25 | 0.29 | 0.18 |
| Neutral | N192W | 624 | 548.30 | 538.86 | 1.02 | 0.08 | 0.06 |
| Up | N192G | 621 | 899.47 | 659.29 | 1.36 | 0.13 | 0.07 |
| Up | N192D | 611 | 4213.33 | 2216.40 | 1.90 | 0.59 | 0.24 |
| Neutral | N192V | 626 | 588.02 | 537.64 | 1.09 | 0.08 | 0.06 |
| Neutral | N192A | 628 | 574.26 | 543.66 | 1.06 | 0.08 | 0.06 |
| Neutral | N192T | 618 | 536.50 | 576.21 | 0.93 | 0.08 | 0.06 |
| Neutral | N192K | 614 | 685.26 | 633.89 | 1.08 | 0.10 | 0.07 |
| Up | N192C | 616 | 1310.46 | 987.31 | 1.33 | 0.18 | 0.11 |
| Neutral | N192M | 623 | 547.98 | 537.29 | 1.02 | 0.08 | 0.06 |
| Neutral | L193P | 2527 | 388.57 | 381.15 | 1.02 | 0.05 | 0.04 |
| Neutral | L193G | 2520 | 437.84 | 478.44 | 0.92 | 0.05 | 0.05 |
| Neutral | L193F | 2521 | 481.49 | 491.33 | 0.98 | 0.06 | 0.05 |
| Neutral | L193S | 2519 | 448.35 | 449.03 | 1.00 | 0.05 | 0.05 |
| Neutral | L193W | 2523 | 481.79 | 460.74 | 1.05 | 0.06 | 0.05 |
| Neutral | L193A | 2526 | 510.96 | 468.83 | 1.09 | 0.06 | 0.05 |
| Neutral | L193R | 2513 | 481.08 | 477.55 | 1.01 | 0.06 | 0.05 |
| Neutral | L193Q | 2516 | 417.53 | 412.01 | 1.01 | 0.05 | 0.05 |
| Neutral | L193E | 2510 | 401.70 | 409.23 | 0.98 | 0.05 | 0.05 |
| Neutral | L193K | 2512 | 417.39 | 426.26 | 0.98 | 0.05 | 0.05 |
| Neutral | L193N | 2515 | 432.35 | 434.95 | 0.99 | 0.05 | 0.05 |
| Down | L193I | 2524 | 2767.03 | 3467.03 | 0.80 | 0.33 | 0.39 |
| Neutral | L193T | 2517 | 679.48 | 638.88 | 1.06 | 0.08 | 0.07 |
| Neutral | L193D | 2509 | 419.37 | 424.41 | 0.99 | 0.05 | 0.05 |
| Neutral | L193Y | 2518 | 3022.00 | 2706.28 | 1.12 | 0.36 | 0.30 |
| Neutral | H194S | 639 | 5518.27 | 6112.38 | 0.90 | 0.78 | 0.67 |
| Neutral | H194E | 631 | 7667.53 | 8295.22 | 0.92 | 1.08 | 0.91 |
| Neutral | H194K | 632 | 5130.62 | 6124.27 | 0.84 | 0.72 | 0.67 |
| Neutral | H194Q | 636 | 6399.62 | 7113.56 | 0.90 | 0.90 | 0.78 |
| Down | H194V | 645 | 1611.06 | 5696.43 | 0.28 | 0.23 | 0.63 |
| Up | H194T | 637 | 3884.64 | 2598.12 | 1.50 | 0.55 | 0.29 |
| Neutral | H194L | 646 | 5710.11 | 6872.56 | 0.83 | 0.80 | 0.76 |
| Neutral | H194Y | 638 | 4922.31 | 5688.29 | 0.87 | 0.69 | 0.63 |
| Down | H194F | 641 | 3787.65 | 5388.18 | 0.70 | 0.53 | 0.59 |
| Neutral | H194G | 640 | 4636.22 | 5437.23 | 0.85 | 0.65 | 0.60 |
| Down | H1941 | 644 | 2901.13 | 3777.68 | 0.77 | 0.41 | 0.42 |
| Down | H194W | 643 | 5434.60 | 7448.23 | 0.73 | 0.77 | 0.82 |
| Down | H194M | 642 | 2941.85 | 9057.43 | 0.32 | 0.41 | 1.00 |
| Up | H194A | 647 | 4681.45 | 2746.90 | 1.70 | 0.66 | 0.30 |
| Neutral | H194P | 648 | 5264.79 | 5058.19 | 1.04 | 0.74 | 0.56 |
| Up | R195C | 273 | 4231.32 | 1853.20 | 2.28 | 0.60 | 0.20 |
| Neutral | R195F | 280 | 687.70 | 720.42 | 0.95 | 0.10 | 0.08 |
| Neutral | R195W | 282 | 5099.23 | 4524.84 | 1.13 | 0.72 | 0.50 |
| Neutral | R195T | 276 | 1101.98 | 1175.85 | 0.94 | 0.16 | 0.13 |
| Neutral | R195L | 285 | 5073.57 | 4520.73 | 1.12 | 0.72 | 0.50 |
| Up | R195G | 279 | 5269.21 | 3025.93 | 1.74 | 0.74 | 0.33 |
| Up | R195Q | 275 | 1958.69 | 1361.83 | 1.44 | 0.28 | 0.15 |
| Down | R195K | 272 | 3839.86 | 7080.78 | 0.54 | 0.54 | 0.78 |
| Neutral | R195S | 278 | 642.14 | 649.21 | 0.99 | 0.09 | 0.07 |
| Up | R195A | 286 | 5605.90 | 3852.81 | 1.46 | 0.79 | 0.42 |
| Up | R195D | 269 | 2724.53 | 1907.81 | 1.43 | 0.38 | 0.21 |
| Neutral | R195P | 287 | 571.50 | 615.21 | 0.93 | 0.08 | 0.07 |
| Neutral | R195Y | 277 | 763.31 | 794.42 | 0.96 | 0.11 | 0.09 |
| Neutral | R195E | 270 | 7597.55 | 8468.35 | 0.90 | 1.07 | 0.93 |
| Up | R19SV | 284 | 1711.48 | 1037.62 | 1.65 | 0.24 | 0.11 |
| Neutral | V196T | 2536 | 1040.90 | 1268.04 | 0.82 | 0.12 | 0.14 |
| Neutral | V196D | 2528 | 443.04 | 446.39 | 0.99 | 0.05 | 0.05 |
| Neutral | V196G | 2539 | 490.83 | 494.67 | 0.99 | 0.06 | 0.06 |
| Neutral | V196E | 2529 | 488.55 | 489.74 | 1.00 | 0.06 | 0.05 |
| Neutral | V196A | 2545 | 452.36 | 452.12 | 1.00 | 0.05 | 0.05 |
| Up | V196S | 2538 | 1186.52 | 949.52 | 1.25 | 0.14 | 0.11 |
| Neutral | V196Q | 2535 | 412.17 | 430.91 | 0.96 | 0.05 | 0.05 |
| Neutral | V196P | 2546 | 576.83 | 620.88 | 0.93 | 0.07 | 0.07 |
| Neutral | V196R | 2532 | 493.29 | 474.38 | 1.04 | 0.06 | 0.05 |
| Neutral | V196H | 2530 | 465.64 | 479.66 | 0.97 | 0.06 | 0.05 |
| Neutral | V196Y | 2537 | 462.28 | 474.94 | 0.97 | 0.06 | 0.05 |
| Neutral | V196I | 2543 | 1125.67 | 1229.87 | 0.92 | 0.13 | 0.14 |
| Neutral | V196L | 2544 | 464.80 | 491.01 | 0.95 | 0.06 | 0.05 |
| Neutral | V196K | 2531 | 455.84 | 482.44 | 0.94 | 0.05 | 0.05 |
| Neutral | V196M | 2541 | 479.36 | 518.00 | 0.93 | 0.06 | 0.06 |
| Down | A197G | 2558 | 1238.39 | 2552.91 | 0.49 | 0.17 | 0.28 |
| Up | A197S | 2557 | 3959.39 | 2633.91 | 1.50 | 0.56 | 0.29 |
| Up | A197L | 2564 | 1013.13 | 809.32 | 1.25 | 0.14 | 0.09 |
| Neutral | A197P | 2565 | 857.06 | 933.29 | 0.92 | 0.12 | 0.10 |
| Down | A197V | 2563 | 2549.12 | 4355.20 | 0.59 | 0.36 | 0.48 |
| Neutral | A197Y | 2556 | 650.21 | 722.49 | 0.90 | 0.09 | 0.08 |
| Neutral | A197Q | 2554 | 658.64 | 652.52 | 1.01 | 0.09 | 0.07 |
| Neutral | A197R | 2551 | 635.08 | 640.91 | 0.99 | 0.09 | 0.07 |
| Down | A197T | 2555 | 1933.94 | 4482.59 | 0.43 | 0.27 | 0.49 |
| Up | A197I | 2562 | 1440.69 | 1060.51 | 1.36 | 0.20 | 0.12 |
| Neutral | A197H | 2549 | 604.11 | 638.63 | 0.95 | 0.09 | 0.07 |
| Neutral | A197E | 2548 | 686.96 | 624.22 | 1.10 | 0.10 | 0.07 |
| Down | A197W | 2561 | 1448.83 | 2588.64 | 0.56 | 0.20 | 0.29 |
| Down | A197N | 2553 | 623.17 | 840.56 | 0.74 | 0.09 | 0.09 |
| Up | A197C | 2552 | 4012.80 | 3140.52 | 1.28 | 0.70 | 0.62 |
| Neutral | A198T | 296 | 761.19 | 700.22 | 1.09 | 0.11 | 0.08 |
| Down | A198K | 291 | 490.20 | 1179.92 | 0.42 | 0.07 | 0.13 |
| Up | A198S | 298 | 4061.28 | 3136.65 | 1.29 | 0.57 | 0.35 |
| Neutral | A198H | 290 | 581.41 | 575.73 | 1.01 | 0.08 | 0.06 |
| Neutral | A198G | 299 | 2610.82 | 2368.26 | 1.10 | 0.37 | 0.26 |
| Down | A198E | 289 | 485.45 | 662.62 | 0.73 | 0.07 | 0.07 |
| Neutral | A198P | 306 | 656.48 | 580.71 | 1.13 | 0.09 | 0.06 |
| Up | A198L | 305 | 1339.94 | 726.74 | 1.84 | 0.19 | 0.08 |
| Neutral | A198R | 292 | 570.33 | 565.56 | 1.01 | 0.08 | 0.06 |
| Down | A198V | 304 | 3026.36 | 7305.85 | 0.41 | 0.43 | 0.80 |
| Up | A198M | 301 | 1384.46 | 999.55 | 1.39 | 0.20 | 0.11 |
| Neutral | A198F | 300 | 572.48 | 559.57 | 1.02 | 0.08 | 0.06 |
| Neutral | A198W | 302 | 560.48 | 547.72 | 1.02 | 0.08 | 0.06 |
| Down | A198Y | 297 | 486.57 | 612.28 | 0.79 | 0.07 | 0.07 |
| Up | A198D | 288 | 633.49 | 474.50 | 1.34 | 0.09 | 0.05 |
| Neutral | H199I | 2580 | 520.35 | 496.48 | 1.05 | 0.08 | 0.07 |
| Neutral | H199P | 2584 | 437.57 | 404.21 | 1.08 | 0.07 | 0.05 |
| Neutral | H199G | 2576 | 436.53 | 392.94 | 1.11 | 0.07 | 0.05 |
| Neutral | H199N | 2571 | 420.26 | 375.18 | 1.12 | 0.07 | 0.05 |
| Neutral | H199S | 2575 | 411.09 | 377.36 | 1.09 | 0.06 | 0.05 |
| Neutral | H199L | 2582 | 531.61 | 530.53 | 1.00 | 0.08 | 0.07 |
| Neutral | H199M | 2578 | 413.37 | 384.23 | 1.08 | 0.07 | 0.05 |
| Neutral | H199A | 2583 | 391.56 | 381.36 | 1.03 | 0.06 | 0.05 |
| Neutral | H199C | 2570 | 404.49 | 366.35 | 1.10 | 0.06 | 0.05 |
| Neutral | H199K | 2568 | 402.34 | 383.95 | 1.05 | 0.06 | 0.05 |
| Neutral | H199R | 2569 | 422.19 | 387.94 | 1.09 | 0.07 | 0.05 |
| Neutral | H199V | 2581 | 421.16 | 378.71 | 1.11 | 0.07 | 0.05 |
| Neutral | H199W | 2579 | 377.01 | 345.02 | 1.09 | 0.06 | 0.05 |
| Neutral | H199T | 2573 | 399.21 | 382.65 | 1.04 | 0.06 | 0.05 |
| Neutral | H199E | 2567 | 399.49 | 385.83 | 1.04 | 0.06 | 0.05 |
| Neutral | E200P | 2603 | 414.11 | 409.55 | 1.01 | 0.07 | 0.06 |
| Neutral | E200G | 2595 | 440.94 | 402.85 | 1.09 | 0.07 | 0.05 |
| Neutral | E200A | 2602 | 448.41 | 413.61 | 1.08 | 0.07 | 0.06 |
| Neutral | E200T | 2592 | 461.19 | 418.51 | 1.10 | 0.07 | 0.06 |
| Neutral | E200I | 2599 | 457.88 | 419.19 | 1.09 | 0.07 | 0.06 |
| Neutral | E200W | 2598 | 418.40 | 403.05 | 1.04 | 0.07 | 0.05 |
| Neutral | E200R | 2588 | 449.83 | 425.86 | 1.06 | 0.07 | 0.06 |
| Neutral | E200F | 2596 | 446.49 | 417.58 | 1.07 | 0.07 | 0.06 |
| Neutral | E200M | 2597 | 448.32 | 428.16 | 1.05 | 0.07 | 0.06 |
| Neutral | E200D | 2585 | 428.91 | 401.64 | 1.07 | 0.07 | 0.05 |
| Neutral | E200V | 2600 | 426.45 | 407.13 | 1.05 | 0.07 | 0.06 |
| Neutral | E200C | 2589 | 413.11 | 384.79 | 1.07 | 0.07 | 0.05 |
| Neutral | E200S | 2594 | 422.57 | 391.02 | 1.08 | 0.07 | 0.05 |
| Neutral | E200Y | 2593 | 412.07 | 393.97 | 1.05 | 0.07 | 0.05 |
| Neutral | E200N | 2590 | 430.94 | 412.07 | 1.05 | 0.07 | 0.06 |
| Down | L201A | 2621 | 754.66 | 957.77 | 0.79 | 0.12 | 0.13 |
| Neutral | L201R | 2608 | 442.35 | 442.57 | 1.00 | 0.07 | 0.06 |
| Neutral | L201E | 2605 | 464.22 | 443.89 | 1.05 | 0.07 | 0.06 |
| Neutral | L201P | 2622 | 494.97 | 471.92 | 1.05 | 0.08 | 0.06 |
| Neutral | L201G | 2615 | 574.82 | 590.26 | 0.97 | 0.09 | 0.08 |
| Down | L201V | 2620 | 3359.21 | 4623.67 | 0.73 | 0.53 | 0.62 |
| Down | L201T | 2612 | 1509.22 | 2175.97 | 0.69 | 0.24 | 0.29 |
| Up | L201I | 2619 | 2861.66 | 2231.87 | 1.28 | 0.45 | 0.30 |
| Neutral | L201S | 2614 | 859.79 | 964.65 | 0.89 | 0.14 | 0.13 |
| Neutral | L201W | 2618 | 1258.36 | 1335.56 | 0.94 | 0.20 | 0.18 |
| Neutral | L201Q | 2611 | 657.51 | 749.98 | 0.88 | 0.10 | 0.10 |
| Neutral | L201D | 2604 | 486.09 | 471.81 | 1.03 | 0.08 | 0.06 |
| Down | L201M | 2617 | 5637.84 | 7147.36 | 0.79 | 0.89 | 0.97 |
| Neutral | L201K | 2607 | 484.89 | 467.23 | 1.04 | 0.08 | 0.06 |
| Neutral | L201N | 2610 | 440.03 | 432.42 | 1.02 | 0.07 | 0.06 |
| Neutral | G202T | 2631 | 556.53 | 546.05 | 1.02 | 0.09 | 0.07 |
| Neutral | G202Y | 2632 | 533.64 | 530.73 | 1.01 | 0.08 | 0.07 |
| Neutral | G202E | 2624 | 558.69 | 543.68 | 1.03 | 0.09 | 0.07 |
| Neutral | G202V | 2638 | 569.22 | 572.58 | 0.99 | 0.09 | 0.08 |
| Neutral | G202S | 2633 | 512.82 | 503.35 | 1.02 | 0.08 | 0.07 |
| Neutral | G202L | 2639 | 513.71 | 508.34 | 1.01 | 0.08 | 0.07 |
| Neutral | G202I | 2637 | 535.96 | 516.37 | 1.04 | 0.08 | 0.07 |
| Neutral | G202M | 2635 | 507.94 | 500.04 | 1.02 | 0.08 | 0.07 |
| Neutral | G202H | 2625 | 567.88 | 547.25 | 1.04 | 0.09 | 0.07 |
| Neutral | G202C | 2628 | 508.19 | 499.05 | 1.02 | 0.08 | 0.07 |
| Neutral | G202R | 2627 | 537.10 | 511.28 | 1.05 | 0.08 | 0.07 |
| Neutral | G202P | 2641 | 544.39 | 535.24 | 1.02 | 0.09 | 0.07 |
| Neutral | G202A | 2640 | 580.75 | 571.95 | 1.02 | 0.09 | 0.08 |
| Neutral | G202K | 2626 | 531.07 | 520.45 | 1.02 | 0.08 | 0.07 |
| Neutral | G202D | 2623 | 559.64 | 544.50 | 1.03 | 0.09 | 0.07 |
| Down | H203Y | 2650 | 910.26 | 1218.02 | 0.75 | 0.14 | 0.16 |
| Neutral | H203E | 2643 | 7284.23 | 7937.91 | 0.92 | 1.15 | 1.07 |
| Neutral | H203R | 2645 | 545.70 | 545.36 | 1.04 | 0.09 | 0.07 |
| Neutral | H203Q | 2648 | 570.55 | 541.42 | 1.05 | 0.09 | 0.07 |
| Neutral | H203P | 2660 | 547.00 | 527.67 | 1.04 | 0.09 | 0.07 |
| Neutral | H203G | 2652 | 558.88 | 576.91 | 0.97 | 0.09 | 0.08 |
| Neutral | H203T | 2649 | 534.16 | 535.33 | 1.00 | 0.08 | 0.07 |
| Neutral | H203D | 2642 | 542.85 | 530.31 | 1.02 | 0.09 | 0.07 |
| Down | H203L | 2658 | 1224.67 | 1746.14 | 0.70 | 0.19 | 0.24 |
| Neutral | H203N | 2647 | 547.92 | 532.11 | 1.03 | 0.09 | 0.07 |
| Neutral | H203A | 2659 | 513.18 | 515.49 | 1.00 | 0.08 | 0.07 |
| Neutral | H203S | 2651 | 534.50 | 507.56 | 1.05 | 0.08 | 0.07 |
| Neutral | H203V | 2657 | 565.64 | 554.43 | 1.02 | 0.09 | 0.07 |
| Neutral | H203I | 2656 | 568.56 | 613.73 | 0.93 | 0.09 | 0.08 |
| Neutral | H203C | 2646 | 504.41 | 522.69 | 0.97 | 0.08 | 0.07 |
| Neutral | S204R | 2665 | 557.42 | 544.69 | 1.02 | 0.09 | 0.07 |
| Neutral | S204N | 2667 | 733.30 | 754.34 | 0.97 | 0.12 | 0.10 |
| Neutral | S204A | 2678 | 3654.83 | 3972.28 | 0.92 | 0.58 | 0.54 |
| Down | S204T | 2669 | 1697.49 | 3586.11 | 0.47 | 0.27 | 0.48 |
| Neutral | S204Y | 2670 | 550.01 | 538.07 | 1.02 | 0.09 | 0.07 |
| Up | S204V | 2676 | 3063.02 | 1827.71 | 1.68 | 0.48 | 0.25 |
| Neutral | S204L | 2677 | 501.10 | 594.44 | 0.84 | 0.08 | 0.08 |
| Neutral | S204H | 2663 | 486.78 | 508.13 | 0.96 | 0.08 | 0.07 |
| Neutral | S204D | 2661 | 507.05 | 489.81 | 1.04 | 0.08 | 0.07 |
| Neutral | S204Q | 2668 | 530.67 | 472.92 | 1.12 | 0.08 | 0.06 |
| Neutral | S204G | 2671 | 1483.41 | 1333.79 | 1.11 | 0.23 | 0.18 |
| Neutral | S204W | 2674 | 487.01 | 504.11 | 0.97 | 0.08 | 0.07 |
| Up | S2041 | 2675 | 634.82 | 516.30 | 1.23 | 0.10 | 0.07 |
| Neutral | S204K | 2664 | 484.92 | 471.83 | 1.03 | 0.08 | 0.06 |
| Neutral | S204P | 2679 | 483.87 | 506.90 | 0.95 | 0.08 | 0.07 |
| Neutral | L205T | 2688 | 1304.89 | 1099.25 | 1.19 | 0.13 | 0.12 |
| Neutral | L205D | 2680 | 774.29 | 830.37 | 0.93 | 0.08 | 0.09 |
| Neutral | L205S | 2690 | 686.11 | 601.35 | 1.14 | 0.07 | 0.07 |
| Neutral | L205G | 2691 | 792.45 | 790.93 | 1.00 | 0.08 | 0.09 |
| Neutral | L205P | 2698 | 592.15 | 673.32 | 0.88 | 0.06 | 0.07 |
| Neutral | L205E | 2681 | 473.89 | 446.64 | 1.06 | 0.05 | 0.05 |
| Down | L20SV | 2696 | 5589.64 | 7308.12 | 0.76 | 0.57 | 0.80 |
| Neutral | L205M | 2693 | 8334.85 | 8229.20 | 1.01 | 0.85 | 0.90 |
| Neutral | L25N | 2686 | 1426.11 | 1322.80 | 1.08 | 0.15 | 0.15 |
| Down | L205C | 2685 | 1903.14 | 2394.15 | 0.79 | 0.20 | 0.26 |
| Down | L205I | 2695 | 5644.28 | 7817.06 | 0.72 | 0.58 | 0.86 |
| Neutral | L205A | 2697 | 1796.22 | 1704.85 | 1.05 | 0.18 | 0.19 |
| Neutral | L20SR | 2684 | 508.62 | 575.22 | 0.88 | 0.05 | 0.06 |
| Neutral | L205W | 2694 | 497.92 | 427.60 | 1.16 | 0.05 | 0.05 |
| Neutral | L205Q | 2687 | 2191.83 | 2399.54 | 0.91 | 0.22 | 0.26 |
| Neutral | G206I | 321 | 467.21 | 460.72 | 1.01 | 0.05 | 0.05 |
| Neutral | G206V | 322 | 619.10 | 682.58 | 0.91 | 0.06 | 0.07 |
| Up | G206A | 324 | 4554.61 | 2702.11 | 1.69 | 0.47 | 0.30 |
| Neutral | G206C | 312 | 491.44 | 469.90 | 1.05 | 0.05 | 0.05 |
| Up | G206S | 317 | 1226.37 | 919.66 | 1.33 | 0.13 | 0.10 |
| Neutral | G206P | 325 | 503.21 | 497.87 | 1.01 | 0.05 | 0.05 |
| Neutral | G206L | 323 | 499.74 | 469.53 | 1.06 | 0.05 | 0.05 |
| Neutral | G206D | 307 | 490.08 | 451.61 | 1.09 | 0.05 | 0.05 |
| Neutral | G206M | 319 | 478.55 | 451.47 | 1.06 | 0.05 | 0.05 |
| Neutral | G206R | 311 | 677.07 | 831.95 | 0.81 | 0.07 | 0.09 |
| Neutral | G206Q | 314 | 805.32 | 851.38 | 0.95 | 0.08 | 0.09 |
| Neutral | G206E | 308 | 469.86 | 447.60 | 1.05 | 0.05 | 0.05 |
| Neutral | G206H | 309 | 463.25 | 437.73 | 1.06 | 0.05 | 0.05 |
| Neutral | G206T | 315 | 475.20 | 491.10 | 0.97 | 0.05 | 0.05 |
| Neutral | G206W | 320 | 472.91 | 437.66 | 1.08 | 0.05 | 0.05 |
| Up | L207S | 659 | 657.07 | 501.03 | 1.31 | 0.07 | 0.05 |
| Neutral | L207Y | 658 | 1032.96 | 1142.52 | 0.90 | 0.11 | 0.13 |
| Neutral | L207A | 666 | 6302.90 | 5614.64 | 1.12 | 0.65 | 0.62 |
| Up | 1207R | 653 | 3476.88 | 1332.44 | 2.61 | 0.36 | 0.15 |
| Neutral | L207P | 667 | 528.87 | 508.95 | 1.04 | 0.05 | 0.06 |
| Up | L207Q | 656 | 671.72 | 518.36 | 1.30 | 0.07 | 0.06 |
| Neutral | L207N | 655 | 551.03 | 476.05 | 1.16 | 0.06 | 0.05 |
| Up | L207K | 652 | 860.90 | 594.92 | 1.45 | 0.09 | 0.07 |
| Neutral | L207M | 662 | 11903.05 | 12984.69 | 0.92 | 1.22 | 1.42 |
| Neutral | L207W | 663 | 509.40 | 470.26 | 1.08 | 0.05 | 0.05 |
| Neutral | L207H | 651 | 620.20 | 595.55 | 1.04 | 0.06 | 0.07 |
| Neutral | L207D | 649 | 523.82 | 473.80 | 1.11 | 0.05 | 0.05 |
| Neutral | L207V | 665 | 656.95 | 550.54 | 1.19 | 0.08 | 0.07 |
| Neutral | L2071 | 664 | 645.37 | 550.32 | 1.17 | 0.08 | 0.07 |
| Up | L207G | 660 | 610.01 | 484.35 | 1.26 | 0.08 | 0.06 |
| Neutral | S208D | 2699 | 10064.82 | 9325.26 | 1.08 | 1.03 | 1.02 |
| Neutral | S208V | 2714 | 10469.49 | 9334.16 | 1.12 | 1.07 | 1.02 |
| Neutral | S208P | 2717 | 9922.26 | 9236.91 | 1.07 | 1.02 | 1.01 |
| Neutral | S208G | 2709 | 10452.64 | 9295.93 | 1.12 | 1.07 | 1.02 |
| Neutral | S208A | 2716 | 10553.22 | 9517.15 | 1.11 | 1.08 | 1.04 |
| Neutral | S208K | 2702 | 22659.58 | 19984.18 | 1.13 | 2.32 | 2.19 |
| Neutral | S208N | 2705 | 9993.85 | 9327.07 | 1.07 | 1.02 | 1.02 |
| Neutral | S208F | 2710 | 8826.28 | 9040.21 | 0.98 | 0.91 | 0.99 |
| Neutral | S208Q | 2706 | 10196.89 | 9183.58 | 1.11 | 1.05 | 1.01 |
| Neutral | S208W | 2712 | 9229.04 | 9226.75 | 1.00 | 0.95 | 1.01 |
| Neutral | S208T | 2707 | 9241.73 | 8912.77 | 1.04 | 0.95 | 0.98 |
| Neutral | S208E | 2700 | 10198.81 | 9401.75 | 1.08 | 1.05 | 1.03 |
| Down | S208C | 2704 | 10497.72 | 16287.64 | 0.64 | 1.08 | 1.79 |
| Neutral | S208R | 2703 | 7639.06 | 6465.10 | 1.18 | 1.34 | 1.28 |
| Up | S208L | 2715 | 7811.78 | 6354.14 | 1.23 | 1.37 | 1.25 |
| Neutral | H2Q9T | 2725 | 466.30 | 415.72 | 1.12 | 0.11 | 0.08 |
| Neutral | H209Y | 2726 | 471.70 | 455.15 | 1.04 | 0.11 | 0.09 |
| Neutral | H209R | 2721 | 489.49 | 463.09 | 1.06 | 0.12 | 0.09 |
| Neutral | H209Q | 2724 | 513.42 | 476.96 | 1.08 | 0.12 | 0.09 |
| Neutral | H209A | 2735 | 511.91 | 469.64 | 1.09 | 0.12 | 0.09 |
| Neutral | H209G | 2728 | 495.58 | 466.25 | 1.06 | 0.12 | 0.09 |
| Neutral | H209N | 2723 | 455.09 | 424.90 | 1.07 | 0.11 | 0.08 |
| Neutral | H209P | 2736 | 526.85 | 480.73 | 1.10 | 0.13 | 0.09 |
| Neutral | H209W | 2731 | 516.05 | 484.16 | 1.07 | 0.12 | 0.09 |
| Neutral | H209V | 2733 | 499.35 | 465.99 | 1.07 | 0.12 | 0.09 |
| Neutral | H209D | 2718 | 479.48 | 442.06 | 1.08 | 0.12 | 0.09 |
| Neutral | H209S | 2727 | 490.77 | 438.98 | 1.12 | 0.12 | 0.09 |
| Neutral | H209F | 2729 | 490.42 | 437.68 | 1.12 | 0.12 | 0.09 |
| Neutral | H209L | 2734 | 491.46 | 441.89 | 1.11 | 0.12 | 0.09 |
| Neutral | H209C | 2722 | 471.56 | 420.60 | 1.12 | 0.11 | 0.08 |
| Neutral | S210C | 331 | 634.06 | 565.38 | 1.12 | 0.15 | 0.11 |
| Neutral | S210G | 336 | 643.08 | 581.11 | 1.11 | 0.16 | 0.11 |
| Up | S210I | 340 | 778.38 | 625.00 | 1.25 | 0.19 | 0.12 |
| Neutral | S210R | 330 | 644.74 | 565.67 | 1.14 | 0.16 | 0.11 |
| Neutral | S210L | 342 | 737.60 | 623.25 | 1.18 | 0.18 | 0.12 |
| Up | S210V | 341 | 1190.35 | 856.63 | 1.39 | 0.29 | 0.17 |
| Neutral | S210H | 328 | 605.43 | 521.90 | 1.16 | 0.15 | 0.10 |
| Neutral | S210N | 332 | 615.29 | 556.38 | 1.11 | 0.15 | 0.11 |
| Neutral | S210F | 337 | 529.93 | 487.42 | 1.09 | 0.13 | 0.09 |
| Neutral | S210P | 344 | 544.94 | 513.59 | 1.06 | 0.13 | 0.10 |
| Neutral | S210W | 339 | 527.32 | 486.97 | 1.08 | 0.13 | 0.09 |
| Neutral | S210Q | 333 | 593.74 | 548.93 | 1.08 | 0.14 | 0.11 |
| Neutral | S210T | 334 | 2977.61 | 3427.71 | 0.87 | 0.72 | 0.67 |
| Neutral | S210K | 329 | 625.14 | 573.41 | 1.09 | 0.15 | 0.11 |
| Neutral | S210A | 343 | 1682.05 | 1546.97 | 1.09 | 0.25 | 0.21 |
| Neutral | T211P | 2755 | 3493.13 | 3774.82 | 0.93 | 0.84 | 0.73 |
| Neutral | T211R | 2741 | 4636.24 | 5429.67 | 0.85 | 1.12 | 1.05 |
| Neutral | T211K | 2740 | 4457.25 | 5411.31 | 0.82 | 1.08 | 1.05 |
| Neutral | T211G | 2747 | 3443.93 | 3543.72 | 0.97 | 0.83 | 0.69 |
| Down | T211M | 2749 | 3806.80 | 4871.37 | 0.78 | 0.92 | 0.95 |
| Neutral | T211N | 2743 | 5924.95 | 6170.25 | 0.96 | 1.43 | 1.20 |
| Neutral | T21JV | 2752 | 5095.76 | 5335.63 | 0.96 | 1.23 | 1.04 |
| Neutral | T211H | 2739 | 1885.69 | 1829.82 | 1.03 | 0.46 | 0.36 |
| Neutral | T211Q | 2744 | 4868.86 | 5772.70 | 0.84 | 1.18 | 1.12 |
| Neutral | T211S | 2746 | 4641.02 | 4565.80 | 1.02 | 1.12 | 0.89 |
| Neutral | T211A | 2754 | 2696.88 | 2830.43 | 0.95 | 0.65 | 0.55 |
| Neutral | T211F | 2748 | 1412.47 | 1277.53 | 1.11 | 0.34 | 0.25 |
| Neutral | T211D | 2737 | 2442.24 | 2154.48 | 1.13 | 0.59 | 0.42 |
| Neutral | T211W | 2750 | 1362.99 | 1207.40 | 1.13 | 0.33 | 0.23 |
| Neutral | T211L | 2753 | 2376.23 | 2102.07 | 1.13 | 0.35 | 0.28 |
| Neutral | D212E | 668 | 4877.68 | 4473.75 | 1.09 | 1.18 | 0.87 |
| Neutral | D212A | 685 | 2710.82 | 2417.65 | 1.12 | 0.66 | 0.47 |
| Neutral | D212K | 670 | 2296.16 | 2049.97 | 1.12 | 0.55 | 0.40 |
| Neutral | D212R | 671 | 2273.87 | 2004.06 | 1.13 | 0.55 | 0.39 |
| Neutral | D212T | 675 | 2923.39 | 2699.75 | 1.08 | 0.71 | 0.52 |
| Neutral | D212N | 673 | 4575.59 | 5229.75 | 0.87 | 1.11 | 1.02 |
| Up | D212G | 678 | 1011.62 | 657.28 | 1.54 | 0.24 | 0.13 |
| Neutral | D212S | 677 | 5035.28 | 4894.92 | 1.03 | 1.22 | 0.95 |
| Neutral | D212P | 686 | 3270.81 | 2918.36 | 1.12 | 0.79 | 0.57 |
| Neutral | D212Q | 674 | 2823.54 | 2576.63 | 1.10 | 0.68 | 0.50 |
| Neutral | D212V | 683 | 2000.60 | 1876.86 | 1.07 | 0.48 | 0.36 |
| Neutral | D212L | 684 | 517.72 | 497.60 | 1.04 | 0.13 | 0.10 |
| Neutral | D212F | 679 | 2378.07 | 2185.27 | 1.09 | 0.57 | 0.42 |
| Neutral | D212H | 669 | 4696.49 | 4001.41 | 1.17 | 0.70 | 0.53 |
| Neutral | D212Y | 676 | 5489.99 | 5319.27 | 1.03 | 0.49 | 0.55 |
| Neutral | I213Q | 2763 | 9326.77 | 8702.80 | 1.07 | 0.96 | 0.95 |
| Neutral | I213T | 2764 | 9396.39 | 8742.82 | 1.07 | 0.96 | 0.96 |
| Neutral | I213C | 2761 | 9396.24 | 8859.11 | 1.06 | 0.96 | 0.97 |
| Neutral | I213P | 2774 | 10248.90 | 9319.88 | 1.10 | 1.05 | 1.02 |
| Neutral | I213H | 2758 | 9826.58 | 9076.23 | 1.08 | 1.01 | 1.00 |
| Neutral | I213A | 2773 | 10044.30 | 9249.07 | 1.09 | 1.03 | 1.01 |
| Neutral | I213V | 2771 | 10260.18 | 9459.80 | 1.08 | 1.05 | 1.04 |
| Neutral | I213G | 2767 | 21327.14 | 19706.88 | 1.08 | 2.19 | 2.16 |
| Neutral | I213N | 2762 | 8790.33 | 7995.03 | 1.10 | 0.90 | 0.88 |
| Neutral | I213L | 2772 | 9974.73 | 9208.92 | 1.08 | 1.02 | 1.01 |
| Neutral | I213S | 2766 | 9599.72 | 9004.65 | 1.07 | 0.98 | 0.99 |
| Neutral | I213M | 2769 | 9987.31 | 9083.77 | 1.10 | 1.02 | 1.00 |
| Neutral | I213R | 2760 | 9253.06 | 8997.34 | 1.03 | 0.95 | 0.99 |
| Neutral | I213K | 2759 | 9682.80 | 9286.32 | 1.04 | 0.99 | 1.02 |
| Neutral | I213F | 2768 | 9368.13 | 8940.38 | 1.05 | 0.96 | 0.98 |
| Neutral | I213D | 2756 | 7017.06 | 7368.43 | 0.95 | 0.77 | 0.97 |
| Neutral | I213E | 2757 | 8169.74 | 7234.77 | 1.13 | 0.90 | 0.95 |
| Neutral | G214L | 2791 | 13500.23 | 13135.46 | 1.03 | 1.38 | 1.44 |
| Neutral | G214Q | 2782 | 9914.48 | 9182.63 | 1.08 | 1.02 | 1.01 |
| Neutral | G214S | 2785 | 9503.68 | 9036.70 | 1.05 | 0.97 | 0.99 |
| Neutral | G214T | 2783 | 9940.86 | 9214.25 | 1.08 | 1.02 | 1.01 |
| Neutral | G214V | 2790 | 8185.36 | 7785.72 | 1.05 | 0.84 | 0.85 |
| Neutral | G2141 | 2789 | 6068.79 | 5773.01 | 1.05 | 0.62 | 0.63 |
| Neutral | G214R | 2779 | 9720.43 | 9083.04 | 1.07 | 1.00 | 1.00 |
| Neutral | G214P | 2793 | 8763.31 | 8875.24 | 0.99 | 0.90 | 0.97 |
| Neutral | G214E | 2776 | 21602.30 | 19851.77 | 1.09 | 2.22 | 2.18 |
| Neutral | G214A | 2792 | 10063.30 | 9154.93 | 1.10 | 1.03 | 1.00 |
| Neutral | G214D | 2775 | 9967.49 | 9121.71 | 1.09 | 1.02 | 1.00 |
| Neutral | G214F | 2786 | 9750.30 | 9157.75 | 1.06 | 1.00 | 1.00 |
| Neutral | G214Y | 2784 | 9886.62 | 9025.45 | 1.10 | 1.01 | 0.99 |
| Neutral | G214M | 2787 | 9472.77 | 8919.05 | 1.06 | 0.97 | 0.98 |
| Neutral | G214C | 2780 | 6716.52 | 7097.60 | 0.95 | 0.69 | 0.78 |
| Neutral | A215L | 2811 | 454.59 | 428.41 | 1.06 | 0.07 | 0.06 |
| Neutral | A215Q | 2801 | 765.06 | 739.09 | 1.04 | 0.12 | 0.10 |
| Neutral | A215M | 2807 | 672.22 | 624.41 | 1.08 | 0.10 | 0.09 |
| Down | A215G | 2805 | 4240.44 | 6854.29 | 0.62 | 0.66 | 0.96 |
| Neutral | A215W | 2808 | 377.79 | 348.04 | 1.09 | 0.06 | 0.05 |
| Neutral | A215S | 2804 | 559.99 | 538.20 | 1.04 | 0.09 | 0.08 |
| Neutral | A215T | 2802 | 664.02 | 711.35 | 0.93 | 0.10 | 0.10 |
| Neutral | A215V | 2810 | 473.67 | 492.63 | 0.96 | 0.07 | 0.07 |
| Neutral | A215N | 2800 | 4328.77 | 4488.89 | 0.96 | 0.67 | 0.63 |
| Neutral | A215P | 2812 | 638.50 | 596.48 | 1.07 | 0.10 | 0.08 |
| Neutral | A215H | 2796 | 3954.04 | 4447.65 | 0.89 | 0.61 | 0.62 |
| Neutral | A215K | 2797 | 420.46 | 402.71 | 1.04 | 0.07 | 0.06 |
| Neutral | A2151 | 2809 | 413.93 | 386.28 | 1.07 | 0.06 | 0.05 |
| Neutral | A215R | 2798 | 421.35 | 389.00 | 1.08 | 0.07 | 0.05 |
| Neutral | A215C | 2799 | 437.44 | 425.03 | 1.03 | 0.07 | 0.06 |
| Neutral | A215D | 2794 | 1031.48 | 913.25 | 1.13 | 0.11 | 0.12 |
| Neutral | L216A | 2830 | 808.93 | 759.54 | 1.07 | 0.13 | 0.12 |
| Neutral | L216C | 2818 | 473.05 | 462.23 | 1.02 | 0.08 | 0.07 |
| Neutral | L216D | 2813 | 497.61 | 457.15 | 1.09 | 0.08 | 0.07 |
| Neutral | L216E | 2814 | 480.72 | 458.21 | 1.05 | 0.08 | 0.07 |
| Neutral | L216G | 2824 | 473.61 | 452.00 | 1.05 | 0.08 | 0.07 |
| Neutral | L216I | 2828 | 7525.06 | 8586.88 | 0.88 | 1.20 | 1.37 |
| Neutral | L216K | 2816 | 478.52 | 460.66 | 1.04 | 0.08 | 0.07 |
| Neutral | L216M | 2826 | 4641.29 | 5160.67 | 0.90 | 0.74 | 0.83 |
| Neutral | L216P | 2831 | 466.46 | 475.96 | 0.98 | 0.07 | 0.08 |
| Neutral | L216Q | 2820 | 693.10 | 638.62 | 1.09 | 0.11 | 0.10 |
| Neutral | L216R | 2817 | 458.77 | 437.34 | 1.05 | 0.07 | 0.07 |
| Neutral | L216S | 2823 | 454.85 | 441.50 | 1.03 | 0.07 | 0.07 |
| Neutral | L216T | 2821 | 1484.74 | 1392.55 | 1.07 | 0.24 | 0.22 |
| Down | L216V | 2829 | 5022.20 | 6281.17 | 0.80 | 0.80 | 1.01 |
| Neutral | L216W | 2827 | 479.55 | 454.07 | 1.06 | 0.08 | 0.07 |
| Neutral | M217P | 2850 | 458.79 | 440.59 | 1.04 | 0.07 | 0.06 |
| Neutral | M217Y | 2841 | 459.96 | 427.87 | 1.07 | 0.07 | 0.06 |
| Neutral | M217T | 2840 | 699.27 | 663.16 | 1.05 | 0.11 | 0.09 |
| Down | M217C | 2837 | 5441.67 | 7486.86 | 0.73 | 0.85 | 1.05 |
| Neutral | M217S | 2842 | 470.92 | 424.03 | 1.11 | 0.07 | 0.06 |
| Neutral | M217L | 2848 | 443.33 | 403.49 | 1.10 | 0.07 | 0.06 |
| Neutral | M217N | 2838 | 462.12 | 424.21 | 1.09 | 0.07 | 0.06 |
| Neutral | M217R | 2836 | 454.58 | 442.20 | 1.03 | 0.07 | 0.06 |
| Neutral | M217Q | 2839 | 449.94 | 427.47 | 1.05 | 0.07 | 0.06 |
| Neutral | M217K | 2835 | 506.96 | 458.74 | 1.11 | 0.08 | 0.06 |
| Neutral | M217G | 2843 | 746.17 | 728.78 | 1.02 | 0.12 | 0.10 |
| Neutral | M217A | 2849 | 437.36 | 410.18 | 1.07 | 0.07 | 0.06 |
| Neutral | M217H | 2834 | 442.18 | 398.29 | 1.11 | 0.07 | 0.06 |
| Neutral | M217I | 2846 | 483.00 | 449.94 | 1.07 | 0.08 | 0.06 |
| Neutral | M217D | 2832 | 503.49 | 491.20 | 1.03 | 0.04 | 0.05 |
| Neutral | Y218C | 350 | 511.72 | 486.65 | 1.05 | 0.08 | 0.07 |
| Down | Y218F | 356 | 4555.92 | 6084.93 | 0.75 | 0.71 | 0.85 |
| Neutral | Y218W | 358 | 8521.86 | 9311.36 | 0.92 | 1.32 | 1.30 |
| Neutral | Y218L | 361 | 834.41 | 743.23 | 1.12 | 0.13 | 0.10 |
| Neutral | Y218A | 362 | 1935.94 | 1652.76 | 1.17 | 0.30 | 0.23 |
| Neutral | Y218P | 363 | 503.58 | 469.06 | 1.07 | 0.08 | 0.07 |
| Neutral | Y218R | 349 | 508.52 | 465.38 | 1.09 | 0.08 | 0.07 |
| Neutral | Y218N | 351 | 704.77 | 640.35 | 1.10 | 0.11 | 0.09 |
| Neutral | Y218V | 360 | 527.03 | 480.30 | 1.10 | 0.08 | 0.07 |
| Neutral | Y218Q | 352 | 513.25 | 468.66 | 1.10 | 0.08 | 0.07 |
| Up | Y218I | 359 | 698.50 | 542.67 | 1.29 | 0.11 | 0.08 |
| Neutral | Y218D | 345 | 835.29 | 885.84 | 0.94 | 0.13 | 0.12 |
| Up | Y218S | 354 | 3702.49 | 3099.73 | 1.19 | 0.58 | 0.43 |
| Neutral | Y218G | 355 | 504.65 | 460.32 | 1.10 | 0.08 | 0.06 |
| Neutral | Y218E | 346 | 511.24 | 471.64 | 1.08 | 0.08 | 0.07 |
| Neutral | P219L | 2868 | 578.24 | 550.18 | 1.05 | 0.09 | 0.08 |
| Neutral | P219C | 2856 | 622.59 | 613.51 | 1.01 | 0.10 | 0.09 |
| Neutral | P219V | 2867 | 586.82 | 583.21 | 1.01 | 0.09 | 0.08 |
| Neutral | P219D | 2851 | 819.59 | 881.94 | 0.93 | 0.13 | 0.12 |
| Neutral | P219F | 2863 | 571.45 | 542.25 | 1.05 | 0.09 | 0.08 |
| Neutral | P219A | 2869 | 1749.52 | 1799.14 | 0.97 | 0.27 | 0.25 |
| Neutral | P219T | 2859 | 870.52 | 853.07 | 1.02 | 0.14 | 0.12 |
| Neutral | P219E | 2852 | 895.73 | 858.50 | 1.04 | 0.14 | 0.12 |
| Neutral | P219Q | 2858 | 601.64 | 557.23 | 1.08 | 0.09 | 0.08 |
| Neutral | P219R | 2855 | 580.05 | 533.83 | 1.09 | 0.09 | 0.07 |
| Neutral | P219H | 2853 | 595.81 | 592.49 | 1.01 | 0.09 | 0.08 |
| Neutral | P219G | 2862 | 625.62 | 619.20 | 1.01 | 0.10 | 0.09 |
| Neutral | P219K | 2854 | 647.47 | 633.20 | 1.02 | 0.10 | 0.09 |
| Neutral | P219S | 2861 | 1549.48 | 1669.93 | 0.93 | 0.24 | 0.23 |
| Neutral | P219W | 2865 | 929.41 | 912.72 | 1.02 | 0.14 | 0.13 |
| Down | S220R | 2874 | 7949.20 | 10460.71 | 0.76 | 1.23 | 1.46 |
| Neutral | S220A | 2887 | 9804.98 | 9347.41 | 1.05 | 1.52 | 1.31 |
| Neutral | S220Q | 2878 | 9804.83 | 9328.79 | 1.05 | 1.52 | 1.30 |
| Neutral | S220T | 2877 | 9371.43 | 9378.23 | 1.00 | 1.46 | 1.31 |
| Down | S220L | 2886 | 1688.62 | 2607.71 | 0.65 | 0.26 | 0.36 |
| Down | S220K | 2873 | 2607.58 | 3704.87 | 0.70 | 0.40 | 0.52 |
| Neutral | S220G | 2880 | 9916.14 | 9356.60 | 1.06 | 1.54 | 1.31 |
| Down | S220H | 2872 | 1496.17 | 1874.12 | 0.80 | 0.23 | 0.26 |
| Neutral | S220E | 2871 | 3553.14 | 3992.18 | 0.89 | 0.55 | 0.56 |
| Neutral | S220M | 2882 | 7913.94 | 8545.54 | 0.93 | 1.23 | 1.20 |
| Neutral | S220V | 2885 | 10179.81 | 9414.03 | 1.08 | 1.58 | 1.32 |
| Neutral | S220P | 2888 | 592.32 | 587.51 | 1.01 | 0.09 | 0.08 |
| Down | S220I | 2884 | 6596.23 | 8678.69 | 0.76 | 1.02 | 1.21 |
| Down | S220F | 2881 | 2458.37 | 3612.28 | 0.68 | 0.38 | 0.51 |
| Neutral | S220N | 2876 | 10548.94 | 9399.76 | 1.12 | 1.64 | 1.31 |
| Up | Y221W | 2902 | 1201.23 | 891.46 | 1.35 | 0.19 | 0.12 |
| Neutral | Y221K | 2892 | 595.72 | 575.31 | 1.04 | 0.09 | 0.08 |
| Neutral | Y221Q | 2896 | 592.45 | 568.96 | 1.04 | 0.09 | 0.08 |
| Neutral | Y221C | 2894 | 583.88 | 558.96 | 1.04 | 0.09 | 0.08 |
| Neutral | Y221N | 2895 | 607.96 | 599.09 | 1.01 | 0.09 | 0.08 |
| Neutral | Y221P | 2907 | 575.23 | 546.02 | 1.05 | 0.09 | 0.08 |
| Neutral | Y221V | 2904 | 600.84 | 608.45 | 0.99 | 0.09 | 0.09 |
| Neutral | Y221A | 2906 | 613.20 | 571.57 | 1.07 | 0.10 | 0.08 |
| Neutral | Y221G | 2899 | 558.30 | 544.78 | 1.02 | 0.09 | 0.08 |
| Neutral | Y221R | 2893 | 508.18 | 483.59 | 1.05 | 0.08 | 0.07 |
| Neutral | Y221S | 2898 | 551.66 | 511.82 | 1.08 | 0.09 | 0.07 |
| Up | Y221M | 2901 | 733.99 | 576.28 | 1.27 | 0.11 | 0.08 |
| Neutral | Y221T | 2897 | 552.92 | 554.37 | 1.00 | 0.09 | 0.08 |
| Neutral | Y221L | 2905 | 600.40 | 544.47 | 1.10 | 0.09 | 0.08 |
| Neutral | Y221E | 2890 | 585.19 | 609.28 | 0.96 | 0.09 | 0.09 |
| Down | T222L | 2924 | 1251.44 | 1749.83 | 0.72 | 0.21 | 0.25 |
| Down | T222Y | 2916 | 3088.86 | 4344.09 | 0.71 | 0.52 | 0.61 |
| Neutral | T222R | 2912 | 7857.83 | 8130.34 | 0.97 | 1.33 | 1.14 |
| Neutral | T222V | 2923 | 6050.08 | 7520.37 | 0.80 | 1.03 | 1.06 |
| Neutral | T222P | 2926 | 9566.57 | 8477.71 | 1.13 | 1.62 | 1.19 |
| Neutral | T222S | 2917 | 8669.64 | 8464.76 | 1.02 | 1.47 | 1.19 |
| Neutral | T222A | 2925 | 5927.34 | 6623.26 | 0.89 | 1.00 | 0.93 |
| Down | T222H | 2910 | 4207.02 | 5413.49 | 0.78 | 0.71 | 0.76 |
| Neutral | T222G | 2918 | 7265.81 | 7630.73 | 0.95 | 1.23 | 1.07 |
| Neutral | T222M | 2920 | 4765.98 | 5354.04 | 0.89 | 0.81 | 0.75 |
| Neutral | T222F | 2919 | 8084.32 | 8023.96 | 1.01 | 1.37 | 1.13 |
| Neutral | T222C | 2913 | 1134.10 | 1047.66 | 1.08 | 0.19 | 0.15 |
| Neutral | T222I | 2922 | 489.93 | 514.09 | 0.95 | 0.08 | 0.07 |
| Neutral | T222N | 2914 | 8082.92 | 8215.81 | 0.98 | 1.37 | 1.15 |
| Down | T222W | 2921 | 4390.84 | 5903.99 | 0.74 | 0.74 | 0.83 |
| Neutral | T222D | 2908 | 4859.28 | 5584.93 | 0.87 | 0.53 | 0.73 |
| Neutral | F223L | 380 | 2776.43 | 3305.29 | 0.84 | 0.47 | 0.46 |
| Neutral | F223T | 372 | 7801.97 | 7792.62 | 1.00 | 1.32 | 1.09 |
| Up | F223C | 369 | 3115.11 | 2488.91 | 1.25 | 0.53 | 0.35 |
| Neutral | F223R | 368 | 5508.50 | 5094.99 | 1.08 | 0.93 | 0.71 |
| Neutral | F223N | 370 | 7434.46 | 6650.94 | 1.12 | 1.26 | 0.93 |
| Neutral | F223P | 382 | 8466.83 | 7678.71 | 1.10 | 1.43 | 1.08 |
| Up | F223E | 365 | 7194.34 | 5884.03 | 1.22 | 1.22 | 0.83 |
| Up | F223G | 375 | 3236.56 | 2599.04 | 1.25 | 0.55 | 0.36 |
| Neutral | F223Q | 371 | 8100.68 | 7468.16 | 1.08 | 1.37 | 1.05 |
| Up | F223A | 381 | 5226.86 | 3982.92 | 1.31 | 0.89 | 0.56 |
| Up | F223S | 374 | 6006.80 | 4916.07 | 1.22 | 1.02 | 0.69 |
| Neutral | F223Y | 373 | 9072.25 | 8479.33 | 1.07 | 1.54 | 1.19 |
| Neutral | F223H | 366 | 8573.59 | 8056.97 | 1.06 | 1.45 | 1.13 |
| Neutral | F223K | 367 | 4021.97 | 3712.91 | 1.08 | 0.60 | 0.49 |
| Neutral | F223M | 376 | 525.66 | 441.29 | 1.19 | 0.08 | 0.06 |
| Neutral | S224G | 2937 | 5580.59 | 6030.81 | 0.93 | 0.89 | 0.97 |
| Neutral | S224T | 2935 | 6189.79 | 7398.93 | 0.84 | 0.99 | 1.18 |
| Neutral | S224Q | 2934 | 7258.89 | 8221.79 | 0.88 | 1.16 | 1.32 |
| Neutral | S224R | 2931 | 4718.67 | 4984.94 | 0.95 | 0.76 | 0.80 |
| Neutral | S224P | 2945 | 475.19 | 459.57 | 1.03 | 0.08 | 0.07 |
| Neutral | S224I | 2941 | 5653.45 | 6319.33 | 0.89 | 0.90 | 1.01 |
| Neutral | S224V | 2942 | 4074.45 | 5042.87 | 0.81 | 0.65 | 0.81 |
| Down | S224L | 2943 | 4272.54 | 5590.35 | 0.76 | 0.68 | 0.89 |
| Neutral | S224C | 2932 | 4057.16 | 4912.59 | 0.83 | 0.65 | 0.79 |
| Neutral | S224K | 2930 | 7286.24 | 8122.32 | 0.90 | 1.17 | 1.30 |
| Neutral | S224D | 2927 | 7201.97 | 8490.41 | 0.85 | 1.15 | 1.36 |
| Neutral | S224H | 2929 | 5928.85 | 6787.33 | 0.87 | 0.95 | 1.09 |
| Neutral | S224M | 2939 | 5967.51 | 6770.07 | 0.88 | 0.95 | 1.08 |
| Neutral | S224A | 2944 | 469.39 | 427.21 | 1.10 | 0.08 | 0.07 |
| Down | S224W | 2940 | 4323.69 | 5971.55 | 0.72 | 0.69 | 0.96 |
| Neutral | G225D | 2946 | 4925.13 | 4615.89 | 1.07 | 0.83 | 0.65 |
| Neutral | G225R | 2950 | 6317.32 | 6775.84 | 0.93 | 1.07 | 0.95 |
| Neutral | G225Q | 2953 | 8693.50 | 8267.06 | 1.05 | 1.47 | 1.16 |
| Neutral | G225M | 2958 | 3626.70 | 3585.88 | 1.01 | 0.61 | 0.50 |
| Neutral | G225P | 2964 | 4775.00 | 4558.87 | 1.05 | 0.81 | 0.64 |
| Neutral | G225W | 2959 | 6452.91 | 7515.31 | 0.86 | 1.09 | 1.05 |
| Neutral | G225S | 2956 | 4811.54 | 4789.30 | 1.00 | 0.82 | 0.67 |
| Neutral | G225E | 2947 | 9174.21 | 8356.85 | 1.10 | 1.55 | 1.17 |
| Neutral | G225V | 2961 | 3525.03 | 3330.02 | 1.06 | 0.60 | 0.47 |
| Neutral | G225T | 2954 | 7463.15 | 7841.71 | 0.95 | 1.26 | 1.10 |
| Neutral | G225K | 2949 | 7135.01 | 7721.15 | 0.92 | 1.21 | 1.08 |
| Neutral | G225N | 2952 | 5858.96 | 5807.35 | 1.01 | 0.99 | 0.81 |
| Neutral | G225C | 2951 | 1631.86 | 1835.77 | 0.89 | 0.28 | 0.26 |
| Neutral | G225H | 2948 | 8719.92 | 8448.61 | 1.03 | 1.48 | 1.19 |
| Neutral | G225A | 2963 | 6048.29 | 5768.91 | 1.05 | 1.03 | 0.81 |
| Neutral | D226S | 2974 | 8608.26 | 8605.77 | 1.00 | 1.46 | 1.21 |
| Neutral | D226W | 2978 | 1817.34 | 2172.37 | 0.84 | 0.31 | 0.30 |
| Down | D226R | 2968 | 5584.63 | 7070.57 | 0.79 | 0.95 | 0.99 |
| Neutral | D226A | 2982 | 6987.67 | 7786.46 | 0.90 | 1.18 | 1.09 |
| Neutral | D226N | 2970 | 6464.01 | 7314.30 | 0.88 | 1.10 | 1.03 |
| Neutral | D226T | 2972 | 3450.45 | 4219.45 | 0.82 | 0.58 | 0.59 |
| Neutral | D226E | 2965 | 9308.62 | 8744.05 | 1.06 | 1.58 | 1.23 |
| Neutral | D226L | 2981 | 3411.80 | 4254.22 | 0.80 | 0.58 | 0.60 |
| Neutral | D226P | 2983 | 8574.77 | 8325.60 | 1.03 | 1.45 | 1.17 |
| Neutral | D226H | 2966 | 4217.71 | 4180.90 | 1.01 | 0.71 | 0.59 |
| Neutral | D226G | 2975 | 6320.31 | 7359.33 | 0.86 | 1.07 | 1.03 |
| Neutral | D226I | 2979 | 7753.45 | 8016.92 | 0.97 | 1.31 | 1.12 |
| Neutral | D226M | 2977 | 6501.53 | 7210.62 | 0.90 | 1.10 | 1.01 |
| Neutral | D226V | 2980 | 3680.55 | 4504.86 | 0.82 | 0.62 | 0.63 |
| Neutral | D226C | 2969 | 7227.15 | 7735.09 | 0.93 | 1.22 | 1.09 |
| Neutral | V227A | 400 | 5109.18 | 5056.19 | 1.01 | 0.87 | 0.71 |
| Up | V227C | 388 | 4040.96 | 3278.65 | 1.23 | 0.68 | 0.46 |
| Up | V227D | 383 | 1190.09 | 731.34 | 1.63 | 0.20 | 0.10 |
| Up | V227E | 384 | 5381.63 | 2605.20 | 2.07 | 0.91 | 0.37 |
| Neutral | V227K | 386 | 580.24 | 550.24 | 1.05 | 0.10 | 0.08 |
| Up | V227L | 399 | 4883.98 | 4000.68 | 1.22 | 0.83 | 0.56 |
| Neutral | V227P | 401 | 3682.22 | 3644.94 | 1.01 | 0.62 | 0.51 |
| Up | V227S | 393 | 3863.33 | 3131.47 | 1.23 | 0.65 | 0.44 |
| Neutral | V227T | 391 | 9817.63 | 8523.33 | 1.15 | 1.66 | 1.20 |
| Up | V227W | 397 | 1845.46 | 1374.06 | 1.34 | 0.31 | 0.19 |
| kn | V227Y | 392 | 657.68 | 542.07 | 1.21 | 0.11 | 0.08 |
| Neutral | V227G | 394 | 1040.74 | 883.01 | 1.18 | 0.15 | 0.12 |
| Up | V227H | 385 | 689.20 | 504.65 | 1.37 | 0.10 | 0.07 |
| Up | V227Q | 390 | 696.97 | 506.11 | 1.38 | 0.10 | 0.07 |
| Neutral | V227R | 387 | 664.31 | 561.06 | 1.18 | 0.10 | 0.07 |
| Neutral | Q228A | 419 | 9710.68 | 9175.62 | 1.06 | 4.50 | 3.13 |
| Neutral | Q228D | 402 | 10931.89 | 9274.00 | 1.18 | 5.06 | 3.16 |
| Neutral | Q228E | 403 | 9825.63 | 9396.31 | 1.05 | 4.55 | 3.20 |
| Neutral | Q228G | 412 | 9400.23 | 9058.34 | 1.04 | 4.35 | 3.09 |
| Neutral | Q228H | 404 | 9748.08 | 9288.74 | 1.05 | 4.51 | 3.17 |
| Neutral | Q228K | 405 | 9999.23 | 9262.11 | 1.08 | 4.63 | 3.16 |
| Neutral | Q228L | 418 | 9199.01 | 8900.02 | 1.03 | 4.26 | 3.03 |
| Neutral | Q228M | 414 | 9510.07 | 8915.79 | 1.07 | 4.40 | 3.04 |
| Neutral | Q228N | 408 | 8774.26 | 8679.09 | 1.01 | 4.06 | 2.96 |
| Up | Q228P | 420 | 2862.74 | 1291.55 | 2.22 | 1.33 | 0.44 |
| Neutral | Q228R | 406 | 7443.02 | 8091.71 | 0.92 | 3.45 | 2.76 |
| Neutral | Q228S | 411 | 8188.30 | 8162.27 | 1.00 | 3.79 | 2.78 |
| Neutral | Q228T | 409 | 4335.26 | 5179.47 | 0.84 | 2.01 | 1.77 |
| Neutral | Q228W | 415 | 6169.39 | 6508.89 | 0.95 | 2.86 | 2.22 |
| Neutral | Q228Y | 410 | 7426.87 | 7840.08 | 0.95 | 3.44 | 2.67 |
| Neutral | L229R | 425 | 485.95 | 489.74 | 0.99 | 0.22 | 0.17 |
| Up | L229A | 438 | 2627.78 | 2118.07 | 1.24 | 1.22 | 0.72 |
| Up | L229T | 429 | 3780.54 | 1464.25 | 2.58 | 1.75 | 0.50 |
| Neutral | L229Q | 428 | 5328.09 | 5303.89 | 1.00 | 2.47 | 1.81 |
| Neutral | L229P | 439 | 4795.14 | 5009.73 | 0.96 | 2.22 | 1.71 |
| Neutral | L229E | 422 | 737.30 | 657.34 | 1.12 | 0.34 | 0.22 |
| Neutral | L229W | 435 | 577.28 | 520.84 | 1.11 | 0.27 | 0.18 |
| Neutral | L229M | 434 | 3207.73 | 2829.20 | 1.13 | 1.49 | 0.96 |
| Up | L229I | 436 | 1158.56 | 828.94 | 1.40 | 0.54 | 0.28 |
| Neutral | L229G | 432 | 552.22 | 520.30 | 1.06 | 0.26 | 0.18 |
| U | L229C | 426 | 633.99 | 516.61 | 1.23 | 0.29 | 0.18 |
| Neutral | L229Y | 430 | 549.90 | 504.60 | 1.09 | 0.25 | 0.17 |
| Neutral | L229D | 421 | 498.06 | 485.50 | 1.03 | 0.23 | 0.17 |
| Neutral | L229H | 423 | 551.54 | 501.77 | 1.10 | 0.26 | 0.17 |
| Neutral | L229V | 437 | 6249.58 | 6487.57 | 0.96 | 2.89 | 2.21 |
| Up | A230L | 704 | 3437.91 | 2154.62 | 1.60 | 1.59 | 0.73 |
| Neutral | A230G | 698 | 6804.87 | 8304.63 | 0.82 | 3.15 | 2.83 |
| Neutral | A230W | 701 | 4773.24 | 5118.69 | 0.93 | 2.21 | 1.74 |
| Up | A230P | 705 | 699.78 | 568.62 | 1.23 | 0.32 | 0.19 |
| Neutral | A230D | 687 | 7281.83 | 8033.59 | 0.91 | 3.37 | 2.74 |
| Up | A230R | 691 | 2986.52 | 2304.10 | 1.30 | 1.38 | 0.79 |
| Up | A2301 | 702 | 4609.64 | 3490.44 | 1.32 | 2.13 | 1.19 |
| Neutral | A230S | 697 | 9181.71 | 8982.15 | 1.02 | 4.25 | 3.06 |
| Neutral | A230C | 692 | 5061.18 | 5781.86 | 0.88 | 2.34 | 1.97 |
| Up | A230V | 703 | 5030.94 | 3433.18 | 1.47 | 2.33 | 1.17 |
| Neutral | A230T | 695 | 8822.74 | 9169.52 | 0.96 | 4.08 | 3.13 |
| Neutral | A230Y | 696 | 3327.47 | 2858.53 | 1.16 | 1.54 | 0.97 |
| Neutral | A230M | 700 | 9543.01 | 9520.33 | 1.00 | 4.42 | 3.25 |
| Neutral | A230N | 693 | 9217.40 | 9384.02 | 0.98 | 4.27 | 3.20 |
| Up | A230H | 689 | 8514.47 | 6763.46 | 1.26 | 3.94 | 2.31 |
| Neutral | Q231I | 2998 | 4161.47 | 3993.24 | 1.04 | 1.93 | 1.36 |
| Neutral | Q231A | 3001 | 6899.50 | 6650.48 | 1.04 | 3.19 | 2.27 |
| Neutral | Q231F | 2995 | 4049.29 | 4120.56 | 0.98 | 1.87 | 1.40 |
| Neutral | Q231P | 3002 | 613.73 | 591.34 | 1.04 | 0.28 | 0.20 |
| Neutral | Q231Y | 2992 | 2460.04 | 2960.32 | 0.83 | 1.14 | 1.01 |
| Down | Q231R | 2988 | 366.16 | 1013.08 | 0.36 | 0.17 | 0.35 |
| Up | Q231L | 3000 | 3744.09 | 2834.26 | 1.32 | 1.73 | 0.97 |
| Neutral | Q231D | 2984 | 7507.92 | 7957.26 | 0.94 | 3.48 | 2.71 |
| Neutral | Q231G | 2994 | 5743.70 | 6012.88 | 0.96 | 2.66 | 2.05 |
| Neutral | Q231V | 2999 | 6114.28 | 6172.88 | 0.99 | 2.83 | 2.10 |
| Neutral | Q231W | 2997 | 4910.80 | 4767.26 | 1.03 | 2.27 | 1.62 |
| Neutral | Q231S | 2993 | 6593.10 | 7180.32 | 0.92 | 3.05 | 2.45 |
| Neutral | Q231H | 2986 | 4961.12 | 5622.05 | 0.88 | 2.30 | 1.92 |
| Up | Q231C | 2989 | 970.74 | 697.37 | 1.39 | 0.45 | 0.24 |
| Down | Q231M | 2996 | 3314.86 | 4166.20 | 0.80 | 1.53 | 1.42 |
| Neutral | D232H | 3004 | 6046.51 | 7174.55 | 0.84 | 2.80 | 2.45 |
| Down | D232G | 3013 | 5492.29 | 7079.04 | 0.78 | 2.54 | 2.41 |
| Neutral | D232R | 3006 | 5077.01 | 5692.86 | 0.89 | 2.35 | 1.94 |
| Neutral | D232P | 3021 | 7665.95 | 8291.54 | 0.92 | 3.55 | 2.83 |
| Neutral | D232Y | 3011 | 3001.62 | 3628.15 | 0.83 | 1.39 | 1.24 |
| Neutral | D232N | 3008 | 825.42 | 739.97 | 1.12 | 0.38 | 0.25 |
| Up | D232S | 3012 | 14389.74 | 5104.26 | 2.82 | 6.66 | 1.74 |
| Neutral | D232F | 3014 | 3599.26 | 3719.64 | 0.97 | 1.67 | 1.27 |
| Neutral | D232V | 3018 | 7938.31 | 9176.75 | 0.87 | 3.68 | 3.13 |
| Neutral | D232K | 3005 | 4844.31 | 5829.58 | 0.83 | 2.24 | 1.99 |
| Neutral | D232W | 3016 | 8404.13 | 9037.60 | 0.93 | 3.89 | 3.08 |
| Neutral | D232Q | 3009 | 7550.58 | 8008.46 | 0.94 | 3.50 | 2.73 |
| Neutral | D232E | 3003 | 9294.39 | 9251.91 | 1.00 | 4.30 | 3.15 |
| Neutral | D232T | 3010 | 9434.20 | 9583.53 | 0.98 | 4.37 | 3.27 |
| Up | D232L | 3019 | 7603.68 | 4213.70 | 1.80 | 3.52 | 1.44 |
| Neutral | D233Q | 446 | 653.34 | 640.95 | 1.02 | 0.30 | 0.22 |
| Neutral | D233P | 458 | 629.51 | 626.42 | 1.00 | 0.29 | 0.21 |
| Neutral | D233S | 449 | 637.89 | 623.07 | 1.02 | 0.30 | 0.21 |
| Neutral | D233T | 447 | 621.24 | 615.06 | 1.01 | 0.29 | 0.21 |
| Neutral | D233A | 457 | 650.58 | 634.46 | 1.03 | 0.30 | 0.22 |
| Neutral | D233W | 453 | 644.19 | 649.94 | 0.99 | 0.30 | 0.22 |
| Neutral | D233G | 450 | 657.96 | 666.27 | 0.99 | 0.30 | 0.23 |
| Up | D233R | 443 | 715.14 | 467.03 | 1.53 | 0.33 | 0.16 |
| Up | D233E | 440 | 2881.17 | 1918.57 | 1.50 | 1.33 | 0.65 |
| Neutral | D233N | 445 | 580.50 | 572.32 | 1.01 | 0.27 | 0.20 |
| Neutral | D233V | 455 | 609.36 | 603.42 | 1.01 | 0.28 | 0.21 |
| Neutral | D233M | 452 | 581.45 | 593.79 | 0.98 | 0.27 | 0.20 |
| Neutral | D233L | 456 | 584.42 | 597.47 | 0.98 | 0.27 | 0.20 |
| Neutral | D233K | 442 | 608.53 | 615.71 | 0.99 | 0.28 | 0.21 |
| Neutral | D2331 | 454 | 682.66 | 661.78 | 1.03 | 0.32 | 0.23 |
| Up | I234A | 476 | 1458.10 | 1018.50 | 1.43 | 0.31 | 0.18 |
| Up | I234T | 467 | 1451.51 | 1188.67 | 1.22 | 0.31 | 0.21 |
| Down | I234V | 474 | 3474.82 | 5245.94 | 0.66 | 0.73 | 0.91 |
| Up | I234W | 473 | 743.35 | 570.02 | 1.30 | 0.16 | 0.10 |
| Up | I234E | 460 | 1301.06 | 840.09 | 1.55 | 0.27 | 0.15 |
| Neutral | I234G | 470 | 498.38 | 467.78 | 1.07 | 0.10 | 0.08 |
| Down | I234L | 475 | 2584.47 | 3312.61 | 0.78 | 0.54 | 0.57 |
| Up | I234H | 461 | 684.95 | 503.63 | 1.36 | 0.14 | 0.09 |
| Down | I234M | 472 | 3478.87 | 4732.62 | 0.74 | 0.73 | 0.82 |
| Up | I234N | 465 | 633.30 | 513.69 | 1.23 | 0.13 | 0.09 |
| Neutral | I234Y | 468 | 749.28 | 930.94 | 0.80 | 0.16 | 0.16 |
| Neutral | I234P | 477 | 470.41 | 431.33 | 1.09 | 0.10 | 0.07 |
| Neutral | I234D | 459 | 428.28 | 397.06 | 1.08 | 0.09 | 0.07 |
| Up | I234Q | 466 | 1095.18 | 837.53 | 1.31 | 0.23 | 0.15 |
| Up | I234C | 464 | 702.09 | 483.22 | 1.45 | 0.15 | 0.08 |
| Neutral | D235H | 3023 | 5217.44 | 6443.75 | 0.81 | 1.10 | 1.12 |
| Neutral | D235G | 3032 | 5966.03 | 6875.44 | 0.87 | 1.26 | 1.19 |
| Down | D235A | 3039 | 5874.20 | 9191.04 | 0.64 | 1.24 | 1.59 |
| Neutral | D235P | 3040 | 488.90 | 464.91 | 1.05 | 0.10 | 0.08 |
| Neutral | D235L | 3038 | 6353.97 | 6868.10 | 0.93 | 1.34 | 1.19 |
| Down | D235V | 3037 | 4167.59 | 6418.00 | 0.65 | 0.88 | 1.11 |
| Neutral | D235E | 3022 | 8377.14 | 8154.10 | 1.03 | 1.76 | 1.41 |
| Neutral | D235R | 3025 | 7249.34 | 7013.16 | 1.03 | 1.53 | 1.22 |
| Neutral | D235Q | 3028 | 6969.55 | 7752.26 | 0.90 | 1.47 | 1.34 |
| Neutral | D235T | 3029 | 6608.45 | 7282.18 | 0.91 | 1.39 | 1.26 |
| Down | D235C | 3026 | 3805.25 | 5237.38 | 0.73 | 0.80 | 0.91 |
| Down | D235S | 3031 | 3798.36 | 6310.00 | 0.60 | 0.80 | 1.09 |
| Neutral | D235N | 3027 | 6427.98 | 6780.43 | 0.95 | 1.35 | 1.18 |
| Neutral | D235Y | 3030 | 3539.06 | 3728.70 | 0.95 | 0.75 | 0.65 |
| Neutral | D2351 | 3036 | 5390.78 | 5499.20 | 0.98 | 1.14 | 0.95 |
| Neutral | G236M | 3053 | 8157.95 | 7452.06 | 1.09 | 1.72 | 1.29 |
| Neutral | G236R | 3045 | 8890.85 | 8115.23 | 1.10 | 1.87 | 1.41 |
| Neutral | G236D | 3041 | 3820.99 | 4576.94 | 0.83 | 0.80 | 0.79 |
| Neutral | G236S | 3051 | 9887.69 | 8558.48 | 1.16 | 2.08 | 1.48 |
| Down | G236T | 3049 | 6244.13 | 7949.24 | 0.79 | 1.32 | 1.38 |
| Neutral | G236C | 3046 | 8441.80 | 7993.25 | 1.06 | 1.78 | 1.39 |
| Neutral | G236K | 3044 | 9473.18 | 8370.29 | 1.13 | 2.00 | 1.45 |
| Neutral | G236E | 3042 | 7240.97 | 7573.43 | 0.96 | 1.53 | 1.31 |
| U | G236P | 3059 | 969.12 | 668.23 | 1.45 | 0.20 | 0.12 |
| Neutral | G236I | 3055 | 5356.96 | 5189.19 | 1.03 | 1.13 | 0.90 |
| Down | G236Y | 3050 | 4511.53 | 5725.61 | 0.79 | 0.95 | 0.99 |
| Neutral | G236L | 3057 | 8099.87 | 7699.72 | 1.05 | 1.71 | 1.34 |
| Down | G236V | 3056 | 4448.72 | 6422.58 | 0.69 | 0.94 | 1.11 |
| Neutral | G236N | 3047 | 8477.40 | 8088.17 | 1.05 | 1.79 | 1.40 |
| Neutral | G236F | 3052 | 5761.86 | 5560.83 | 1.04 | 1.21 | 0.96 |
| Neutral | I237S | 3070 | 587.31 | 536.19 | 1.10 | 0.12 | 0.09 |
| Up | I237L | 3076 | 2880.14 | 2240.61 | 1.29 | 0.61 | 0.39 |
| Neutral | I237R | 3064 | 572.70 | 548.66 | 1.04 | 0.12 | 0.10 |
| Neutral | I237Q | 3067 | 552.21 | 543.69 | 1.02 | 0.12 | 0.09 |
| Neutral | I237K | 3063 | 571.56 | 531.05 | 1.08 | 0.12 | 0.09 |
| Neutral | I237D | 3060 | 567.39 | 512.64 | 1.11 | 0.12 | 0.09 |
| Down | I237A | 3077 | 1512.93 | 2138.58 | 0.71 | 0.32 | 0.37 |
| Neutral | I237T | 3068 | 572.40 | 524.74 | 1.09 | 0.12 | 0.09 |
| Neutral | I237E | 3061 | 555.97 | 535.94 | 1.04 | 0.12 | 0.09 |
| Neutral | I237C | 3065 | 565.11 | 532.36 | 1.06 | 0.12 | 0.09 |
| Neutral | I237G | 3071 | 620.23 | 586.38 | 1.06 | 0.13 | 0.10 |
| Neutral | I237P | 3078 | 554.04 | 497.24 | 1.11 | 0.12 | 0.09 |
| Neutral | I237Y | 3069 | 688.92 | 602.65 | 1.14 | 0.15 | 0.10 |
| Down | I237W | 3074 | 4188.38 | 5663.94 | 0.74 | 0.62 | 0.75 |
| Neutral | I237N | 3066 | 5368.49 | 6271.59 | 0.86 | 0.80 | 0.83 |
| Down | Q238G | 3089 | 1382.45 | 2524.64 | 0.55 | 0.29 | 0.44 |
| Down | Q238H | 3081 | 3150.20 | 5045.01 | 0.62 | 0.66 | 0.88 |
| Down | Q238S | 3088 | 3298.60 | 4524.89 | 0.73 | 0.69 | 0.78 |
| Down | Q238Y | 3087 | 2078.90 | 2953.44 | 0.70 | 0.44 | 0.51 |
| Down | Q238F | 3090 | 1342.33 | 1916.87 | 0.70 | 0.28 | 0.33 |
| Down | Q238E | 3080 | 4075.95 | 5719.51 | 0.71 | 0.86 | 0.99 |
| Down | Q238L | 3095 | 3030.44 | 4771.52 | 0.64 | 0.64 | 0.83 |
| Neutral | Q238W | 3092 | 3649.81 | 4317.00 | 0.85 | 0.77 | 0.75 |
| Neutral | Q238P | 3097 | 568.68 | 548.50 | 1.04 | 0.12 | 0.10 |
| Down | Q238R | 3083 | 4199.78 | 5952.76 | 0.71 | 0.88 | 1.03 |
| Down | Q238C | 3084 | 3179.60 | 4072.46 | 0.78 | 0.67 | 0.71 |
| Neutral | Q238N | 3085 | 3119.13 | 3894.89 | 0.80 | 0.66 | 0.68 |
| Down | Q238I | 3093 | 3863.21 | 5191.15 | 0.74 | 0.81 | 0.90 |
| Neutral | Q238T | 3086 | 7425.48 | 7998.67 | 0.93 | 1.56 | 1.39 |
| Down | Q238K | 3082 | 4717.87 | 5952.01 | 0.79 | 0.99 | 1.03 |
| Neutral | A239S | 3108 | 7235.02 | 7630.43 | 0.95 | 1.52 | 1.32 |
| Down | A239Q | 3105 | 4817.25 | 8226.13 | 0.59 | 1.01 | 1.43 |
| Down | A239T | 3106 | 1232.13 | 2351.52 | 0.52 | 0.26 | 0.41 |
| Neutral | A239P | 3116 | 606.39 | 564.14 | 1.07 | 0.13 | 0.10 |
| Neutral | A239V | 3114 | 6075.94 | 6919.38 | 0.88 | 1.28 | 1.20 |
| Neutral | A239L | 3115 | 7174.28 | 7878.58 | 0.91 | 1.51 | 1.37 |
| Neutral | A239Y | 3107 | 5570.87 | 6668.58 | 0.84 | 1.17 | 1.16 |
| Neutral | A239I | 3113 | 6821.36 | 7628.60 | 0.89 | 1.44 | 1.32 |
| Neutral | A239C | 3103 | 4986.74 | 5916.10 | 0.84 | 1.05 | 1.03 |
| Neutral | A239G | 3109 | 6430.98 | 7617.20 | 0.84 | 1.35 | 1.32 |
| Down | A239W | 3112 | 2215.28 | 4554.04 | 0.49 | 0.47 | 0.79 |
| Neutral | A239F | 3110 | 719.92 | 750.33 | 0.96 | 0.15 | 0.13 |
| Neutral | A239K | 3101 | 8365.39 | 8161.53 | 1.02 | 1.76 | 1.42 |
| Neutral | A239H | 3100 | 6013.93 | 6892.53 | 0.87 | 1.27 | 1.20 |
| Neutral | A239R | 3102 | 8860.20 | 8322.26 | 1.06 | 1.87 | 1.44 |
| Neutral | A239D | 3098 | 9256.74 | 8197.40 | 1.13 | 1.02 | 1.08 |
| Up | 1240G | 489 | 550.59 | 455.20 | 1.21 | 0.09 | 0.06 |
| Neutral | 1240Q | 485 | 1050.40 | 921.68 | 1.14 | 0.16 | 0.12 |
| Down | 1240P | 496 | 2259.38 | 3251.71 | 0.69 | 0.35 | 0.42 |
| Down | 1240R | 482 | 2771.00 | 3465.26 | 0.80 | 0.43 | 0.44 |
| Up | 1240S | 488 | 2033.91 | 1204.66 | 1.69 | 0.32 | 0.15 |
| Neutral | 1240K | 481 | 5557.21 | 6183.54 | 0.90 | 0.87 | 0.79 |
| Down | 1240V | 493 | 4682.76 | 6307.59 | 0.74 | 0.73 | 0.81 |
| Neutral | 1240D | 478 | 480.83 | 456.39 | 1.05 | 0.08 | 0.06 |
| Neutral | 1240A | 495 | 2099.13 | 1776.41 | 1.18 | 0.33 | 0.23 |
| Up | 1240C | 483 | 970.78 | 650.04 | 1.49 | 0.15 | 0.08 |
| Neutral | 1240L | 494 | 8303.04 | 8506.66 | 0.98 | 1.30 | 1.09 |
| Down | 1240F | 490 | 1345.29 | 2090.14 | 0.64 | 0.21 | 0.27 |
| Up | 1240Y | 487 | 1910.61 | 1482.53 | 1.29 | 0.30 | 0.19 |
| Neutral | 1240M | 491 | 8056.10 | 7463.56 | 1.08 | 1.26 | 0.95 |
| Neutral | 1240T | 486 | 2147.14 | 1862.29 | 1.15 | 0.34 | 0.24 |
| Neutral | Y241V | 3132 | 568.18 | 567.15 | 1.00 | 0.09 | 0.07 |
| Neutral | Y241A | 3134 | 514.00 | 498.25 | 1.03 | 0.08 | 0.06 |
| Neutral | Y241G | 3127 | 484.83 | 493.78 | 0.98 | 0.08 | 0.06 |
| Neutral | Y241H | 3119 | 555.41 | 547.86 | 1.01 | 0.09 | 0.07 |
| Neutral | Y241R | 3121 | 479.35 | 491.61 | 0.98 | 0.08 | 0.06 |
| Neutral | Y241P | 3135 | 542.62 | 468.74 | 1.16 | 0.09 | 0.06 |
| Neutral | Y241Q | 3124 | 494.42 | 468.65 | 1.05 | 0.08 | 0.06 |
| Neutral | Y241L | 3133 | 486.82 | 484.95 | 1.00 | 0.08 | 0.06 |
| Neutral | Y241T | 3125 | 574.08 | 548.16 | 1.05 | 0.09 | 0.07 |
| Neutral | Y241S | 3126 | 512.62 | 498.70 | 1.03 | 0.08 | 0.06 |
| Neutral | Y241W | 3130 | 592.83 | 556.32 | 1.07 | 0.09 | 0.07 |
| Neutral | Y241N | 3123 | 438.44 | 443.51 | 0.99 | 0.07 | 0.06 |
| Neutral | Y241M | 3129 | 488.24 | 448.13 | 1.09 | 0.08 | 0.06 |
| Neutral | Y241I | 3131 | 469.78 | 446.31 | 1.05 | 0.07 | 0.06 |
| Neutral | Y241D | 3117 | 454.99 | 443.57 | 1.03 | 0.07 | 0.06 |
| Neutral | G242A | 3153 | 1948.43 | 1965.86 | 0.99 | 0.31 | 0.25 |
| Neutral | G242F | 3147 | 2367.13 | 2861.31 | 0.83 | 0.37 | 0.37 |
| Down | G242L | 3152 | 4261.36 | 6043.02 | 0.71 | 0.67 | 0.77 |
| Neutral | G242N | 3142 | 1712.51 | 1893.88 | 0.90 | 0.27 | 0.24 |
| Neutral | C242P | 3154 | 2683.19 | 3311.87 | 0.81 | 0.42 | 0.42 |
| Down | C242W | 3149 | 1200.76 | 1522.34 | 0.79 | 0.19 | 0.19 |
| Neutral | G242T | 3144 | 1845.15 | 1926.23 | 0.96 | 0.29 | 0.25 |
| Neutral | G242R | 3140 | 1425.79 | 1462.84 | 0.97 | 0.22 | 0.19 |
| Neutral | G242V | 3151 | 1864.42 | 2075.51 | 0.90 | 0.29 | 0.27 |
| Down | G242S | 3146 | 3463.26 | 4491.54 | 0.77 | 0.54 | 0.57 |
| Down | G242I | 3150 | 881.04 | 2441.87 | 0.36 | 0.14 | 0.31 |
| Neutral | G242Y | 3145 | 895.61 | 928.34 | 0.96 | 0.14 | 0.12 |
| Neutral | G242H | 3138 | 1038.60 | 1063.68 | 0.98 | 0.16 | 0.14 |
| Neutral | G242E | 3137 | 1039.40 | 1200.19 | 0.87 | 0.16 | 0.15 |
| Neutral | G242K | 3139 | 1259.85 | 1404.80 | 0.90 | 0.20 | 0.18 |
| Down | R243P | 3173 | 3936.04 | 7438.61 | 0.53 | 0.62 | 0.95 |
| Neutral | R243K | 3158 | 8397.44 | 8514.77 | 0.99 | 1.32 | 1.09 |
| Neutral | R243T | 3162 | 7451.28 | 7306.32 | 1.02 | 1.17 | 0.93 |
| Neutral | R243L | 3171 | 6953.44 | 7458.28 | 0.93 | 1.09 | 0.95 |
| Neutral | R243A | 3172 | 8253.02 | 8378.15 | 0.99 | 1.29 | 1.07 |
| Neutral | R243H | 3157 | 6757.06 | 7710.25 | 0.88 | 1.06 | 0.99 |
| Neutral | R243Q | 3161 | 7563.55 | 8367.33 | 0.90 | 1.19 | 1.07 |
| Neutral | R243S | 3164 | 7872.26 | 8367.98 | 0.94 | 1.23 | 1.07 |
| Down | R243I | 3169 | 4421.12 | 8337.68 | 0.53 | 0.69 | 1.07 |
| Neutral | R243C | 3159 | 6128.24 | 6907.63 | 0.89 | 0.96 | 0.88 |
| Neutral | R243N | 3160 | 7064.71 | 7808.36 | 0.90 | 1.11 | 1.00 |
| Neutral | R243Y | 3163 | 6415.10 | 7427.20 | 0.86 | 1.01 | 0.95 |
| Neutral | R243G | 3165 | 9279.36 | 8697.39 | 1.07 | 1.46 | 1.11 |
| Neutral | R243D | 3155 | 5769.78 | 6318.28 | 0.91 | 0.90 | 0.81 |
| Neutral | R243V | 3170 | 6349.88 | 7531.41 | 0.84 | 1.00 | 0.96 |
| Neutral | S244P | 3192 | 7394.47 | 7844.16 | 0.94 | 1.16 | 1.00 |
| Down | S244L | 3190 | 3480.19 | 7154.31 | 0.49 | 0.55 | 0.91 |
| Neutral | S244W | 3187 | 10346.96 | 9035.98 | 1.15 | 1.62 | 1.15 |
| Neutral | S244M | 3186 | 728.14 | 748.02 | 0.97 | 0.11 | 0.10 |
| Neutral | S244V | 3189 | 6842.04 | 7456.02 | 0.92 | 1.07 | 0.95 |
| Neutral | S244Q | 3181 | 9318.66 | 8746.49 | 1.07 | 1.46 | 1.12 |
| Neutral | S244D | 3174 | 6915.01 | 7609.44 | 0.91 | 1.08 | 0.97 |
| Neutral | S244E | 3175 | 8814.39 | 8156.16 | 1.08 | 1.38 | 1.04 |
| Neutral | S244T | 3182 | 7442.34 | 8205.48 | 0.91 | 1.17 | 1.05 |
| Down | S244H | 3176 | 5019.42 | 8528.78 | 0.59 | 0.79 | 1.09 |
| Neutral | S244G | 3184 | 888.96 | 801.21 | 1.11 | 0.14 | 0.10 |
| Neutral | S244A | 3191 | 9174.68 | 8474.43 | 1.08 | 1.44 | 1.08 |
| Neutral | S244F | 3185 | 962.91 | 1017.49 | 0.95 | 0.15 | 0.13 |
| Neutral | S244Y | 3183 | 6333.20 | 6595.86 | 0.96 | 0.99 | 0.84 |
| Neutral | S244R | 3178 | 10483.23 | 9488.64 | 1.10 | 0.93 | 0.99 |
| Neutral | Q245P | 3211 | 8046.62 | 8690.91 | 0.93 | 1.26 | 1.11 |
| Neutral | Q245I | 3207 | 7611.43 | 8270.47 | 0.92 | 1.19 | 1.06 |
| Down | Q245F | 3204 | 3940.03 | 8048.83 | 0.49 | 0.62 | 1.03 |
| Neutral | Q245V | 3208 | 7785.27 | 8186.90 | 0.95 | 1.22 | 1.05 |
| Up | Q245M | 3205 | 494.18 | 323.62 | 1.53 | 0.08 | 0.04 |
| Neutral | Q245T | 3200 | 8684.29 | 8676.53 | 1.00 | 1.36 | 1.11 |
| Neutral | Q245E | 3194 | 10044.47 | 8646.78 | 1.16 | 1.58 | 1.10 |
| Neutral | Q245S | 3202 | 8700.39 | 8695.56 | 1.00 | 1.36 | 1.11 |
| Neutral | Q245R | 3197 | 8323.06 | 8629.37 | 0.96 | 1.31 | 1.10 |
| Neutral | Q245G | 3203 | 8495.47 | 8561.87 | 0.99 | 1.33 | 1.09 |
| Neutral | Q245H | 3195 | 8236.63 | 8640.32 | 0.95 | 1.29 | 1.10 |
| Neutral | Q245L | 3209 | 6762.99 | 6774.37 | 1.00 | 1.06 | 0.87 |
| Neutral | Q245K | 3196 | 347.86 | 290.28 | 1.20 | 0.05 | 0.04 |
| Neutral | Q245W | 3206 | 7517.93 | 8157.63 | 0.92 | 1.18 | 1.04 |
| Neutral | Q245C | 3198 | 7377.19 | 7707.00 | 0.96 | 1.16 | 0.98 |
| Down | N246W | 3225 | 3998.57 | 5256.41 | 0.76 | 0.55 | 0.68 |
| Neutral | N246R | 3216 | 6324.43 | 7263.78 | 0.87 | 0.87 | 0.93 |
| Neutral | N246A | 3229 | 7162.60 | 7821.64 | 0.92 | 0.98 | 1.00 |
| Neutral | N246F | 3223 | 5961.78 | 6704.16 | 0.89 | 0.82 | 0.86 |
| Neutral | N246G | 3222 | 7132.52 | 7954.86 | 0.90 | 0.98 | 1.02 |
| Neutral | N246P | 3230 | 5753.82 | 6382.30 | 0.90 | 0.79 | 0.82 |
| Neutral | N246V | 327 | 7113.82 | 7563.12 | 0.94 | 0.98 | 0.97 |
| Neutral | N246Q | 3218 | 8249.22 | 7962.93 | 1.04 | 1.13 | 1.02 |
| Neutral | N246Y | 3220 | 6460.04 | 7091.47 | 0.91 | 0.89 | 0.91 |
| Neutral | N246C | 3217 | 3668.07 | 4131.97 | 0.89 | 0.50 | 0.53 |
| Neutral | N246I | 3226 | 6761.84 | 6684.68 | 1.01 | 0.93 | 0.86 |
| Neutral | N246L | 3228 | 7470.87 | 7558.25 | 0.99 | 1.03 | 0.97 |
| Neutral | N246S | 3221 | 7681.59 | 7872.41 | 0.98 | 1.05 | 1.01 |
| Neutral | N246T | 3219 | 7476.51 | 7504.31 | 1.00 | 1.03 | 0.96 |
| Neutral | N246K | 3215 | 8008.77 | 7820.31 | 1.02 | 1.10 | 1.00 |
| Neutral | N246D | 3212 | 7062.38 | 7827.99 | 0.90 | 0.78 | 1.03 |
| Neutral | P247A | 3249 | 8242.00 | 7947.96 | 1.04 | 1.13 | 1.02 |
| Neutral | P247D | 3231 | 6640.01 | 7179.97 | 0.92 | 0.91 | 0.92 |
| Neutral | P247E | 3232 | 8181.45 | 7231.43 | 1.13 | 1.12 | 0.93 |
| Neutral | P247F | 3243 | 8964.42 | 7929.76 | 1.13 | 1.23 | 1.02 |
| Neutral | P247G | 3242 | 7256.65 | 7455.20 | 0.97 | 1.00 | 0.96 |
| Neutral | P247H | 3233 | 8093.84 | 7667.72 | 1.06 | 1.11 | 0.99 |
| Neutral | P247I | 3246 | 7375.24 | 7729.05 | 0.95 | 1.01 | 0.99 |
| Neutral | P247K | 3234 | 8454.74 | 7912.16 | 1.07 | 1.16 | 1.02 |
| Neutral | P247L | 3248 | 8316.29 | 8009.70 | 1.04 | 1.14 | 1.03 |
| Neutral | P247N | 3237 | 8142.72 | 8006.73 | 1.02 | 1.12 | 1.03 |
| Neutral | P247Q | 3238 | 8231.43 | 7739.72 | 1.06 | 1.13 | 0.99 |
| Neutral | P247R | 3235 | 7029.19 | 7443.10 | 0.94 | 0.96 | 0.96 |
| Neutral | P247S | 3241 | 8040.91 | 7895.89 | 1.02 | 1.10 | 1.01 |
| Neutral | P247T | 3239 | 7243.03 | 7527.94 | 0.96 | 0.99 | 0.97 |
| Neutral | P247V | 3247 | 7907.19 | 7717.20 | 1.02 | 1.08 | 0.99 |
| Neutral | V248W | 3264 | 6631.29 | 6916.87 | 0.96 | 0.91 | 0.89 |
| Neutral | V248L | 3266 | 8767.88 | 8252.54 | 1.06 | 1.20 | 1.06 |
| Neutral | V248Q | 3257 | 6709.44 | 6735.33 | 1.00 | 0.92 | 0.87 |
| Neutral | V248M | 3263 | 7437.73 | 7338.43 | 1.01 | 1.02 | 0.94 |
| Neutral | V248Y | 3259 | 6509.63 | 6927.69 | 0.94 | 0.89 | 0.89 |
| Neutral | V248G | 3261 | 6438.97 | 6744.48 | 0.95 | 0.88 | 0.87 |
| Neutral | V248C | 3255 | 3692.16 | 3816.99 | 0.97 | 0.51 | 0.49 |
| Neutral | V248R | 3254 | 7253.81 | 7153.37 | 1.01 | 1.00 | 0.92 |
| Neutral | V248A | 3267 | 8420.73 | 7739.03 | 1.09 | 1.16 | 0.99 |
| Neutral | V248H | 3252 | 8578.28 | 7867.88 | 1.09 | 1.18 | 1.01 |
| Neutral | V248I | 3265 | 8473.10 | 8209.35 | 1.03 | 1.16 | 1.05 |
| Neutral | V248T | 3258 | 8100.54 | 7751.03 | 1.05 | 1.11 | 1.00 |
| Neutral | V248K | 3253 | 7147.53 | 7653.87 | 0.93 | 0.98 | 0.98 |
| Neutral | V248S | 3260 | 6899.65 | 6729.67 | 1.03 | 0.95 | 0.86 |
| Neutral | V248F | 3262 | 6736.68 | 6651.01 | 1.01 | 0.92 | 0.85 |
| Neutral | V248E | 3251 | 9210.34 | 8235.44 | 1.12 | 1.01 | 1.08 |
| Neutral | Q249T | 3276 | 6909.47 | 7370.27 | 0.94 | 0.95 | 0.95 |
| Neutral | Q249W | 3282 | 10145.55 | 8691.89 | 1.17 | 1.39 | 1.12 |
| Neutral | Q249R | 373 | 7102.73 | 7103.74 | 1.00 | 0.97 | 0.91 |
| Down | Q249E | 3270 | 3987.22 | 7583.88 | 0.53 | 0.55 | 0.97 |
| Neutral | Q249A | 3286 | 8992.77 | 8414.65 | 1.07 | 1.23 | 1.08 |
| Neutral | Q249P | 3287 | 8376.56 | 8108.11 | 1.03 | 1.15 | 1.04 |
| Neutral | Q249C | 3274 | 5978.93 | 5496.03 | 1.09 | 0.82 | 0.71 |
| Neutral | Q249G | 3279 | 7612.71 | 7662.25 | 0.99 | 1.04 | 0.98 |
| Neutral | Q249N | 3275 | 7180.54 | 7257.66 | 0.99 | 0.99 | 0.93 |
| Neutral | Q249K | 3272 | 7772.72 | 7296.54 | 1.07 | 1.07 | 0.94 |
| Neutral | Q249I | 3283 | 7262.56 | 7159.06 | 1.01 | 1.00 | 0.92 |
| Neutral | Q249Y | 3277 | 6047.16 | 6053.08 | 1.00 | 0.83 | 0.78 |
| Neutral | Q249V | 3284 | 8717.93 | 8059.04 | 1.08 | 1.20 | 1.04 |
| Neutral | Q249L | 3285 | 6532.65 | 6824.78 | 0.96 | 0.90 | 0.88 |
| Neutral | Q249H | 3271 | 8441.70 | 7557.69 | 1.12 | 1.16 | 0.97 |
| Neutral | P250L | 3305 | 9455.47 | 8580.12 | 1.10 | 1.30 | 1.10 |
| Neutral | P250S | 3298 | 7684.90 | 7513.77 | 1.02 | 1.05 | 0.97 |
| Neutral | P250R | 3292 | 7701.81 | 7566.23 | 1.02 | 1.06 | 0.97 |
| Neutral | P2S0Y | 3297 | 7886.68 | 7534.22 | 1.05 | 1.08 | 0.97 |
| Neutral | P2S0M | 3301 | 8416.52 | 8221.73 | 1.02 | 1.15 | 1.06 |
| Neutral | P2S0F | 3300 | 8150.35 | 7703.24 | 1.06 | 1.12 | 0.99 |
| Neutral | P250A | 3306 | 8963.20 | 8460.94 | 1.06 | 1.23 | 1.09 |
| Neutral | P250K | 3291 | 7830.18 | 7732.04 | 1.01 | 1.07 | 0.99 |
| Neutral | P250G | 3299 | 7623.88 | 7834.34 | 0.97 | 1.05 | 1.01 |
| Neutral | P250N | 3294 | 7600.44 | 7961.88 | 0.95 | 1.04 | 1.02 |
| Down | P250T | 3296 | 1147.37 | 1489.99 | 0.77 | 0.16 | 0.19 |
| Neutral | P250W | 3302 | 7431.76 | 7755.84 | 0.96 | 1.02 | 1.00 |
| Neutral | P2S0D | 3288 | 7767.77 | 7525.09 | 1.03 | 1.07 | 0.97 |
| Neutral | P2S0V | 3304 | 7355.32 | 7719.82 | 0.95 | 1.01 | 0.99 |
| Neutral | P250Q | 3295 | 7797.52 | 8203.80 | 0.95 | 1.07 | 1.05 |
| Down | I251A | 3324 | 4953.41 | 8984.79 | 0.55 | 0.48 | 0.96 |
| Neutral | I251Q | 3314 | 10910.92 | 9221.40 | 1.18 | 1.07 | 0.98 |
| Neutral | I251G | 3318 | 11041.83 | 9640.57 | 1.15 | 1.08 | 1.03 |
| Neutral | I251L | 3323 | 11028.53 | 9408.72 | 1.17 | 1.08 | 1.00 |
| Neutral | I251K | 3310 | 11050.61 | 9421.73 | 1.17 | 1.08 | 1.01 |
| Neutral | I251R | 3311 | 10950.25 | 9220.62 | 1.19 | 1.07 | 0.98 |
| Neutral | I251E | 3308 | 10262.05 | 9115.62 | 1.13 | 1.00 | 0.97 |
| Neutral | I251D | 3307 | 10582.82 | 9557.71 | 1.11 | 1.04 | 1.02 |
| Neutral | I251T | 3315 | 10884.22 | 9485.20 | 1.15 | 1.06 | 1.01 |
| Neutral | I251C | 3312 | 10348.04 | 9428.04 | 1.10 | 1.01 | 1.01 |
| Neutral | I251Y | 3316 | 10319.00 | 9450.22 | 1.09 | 1.01 | 1.01 |
| Neutral | I251P | 3325 | 10762.38 | 9410.57 | 1.14 | 1.05 | 1.00 |
| Neutral | I251S | 3317 | 8445.88 | 7160.96 | 1.18 | 1.07 | 0.96 |
| Neutral | I251W | 3321 | 7305.95 | 6974.26 | 1.05 | 0.92 | 0.93 |
| Neutral | I251V | 3322 | 8343.83 | 7350.61 | 1.14 | 0.91 | 0.98 |
| Neutral | G252F | 3337 | 7921.80 | 7529.24 | 1.05 | 1.09 | 0.97 |
| Neutral | G252W | 3339 | 6989.36 | 7313.18 | 0.96 | 0.96 | 0.94 |
| Neutral | G252A | 3343 | 8567.46 | 8300.90 | 1.03 | 1.18 | 1.07 |
| Neutral | G252R | 3330 | 7756.55 | 7447.08 | 1.04 | 1.06 | 0.96 |
| Neutral | G252L | 3342 | 8684.63 | 8094.21 | 1.07 | 1.19 | 1.04 |
| Neutral | G252E | 3327 | 7651.86 | 7211.52 | 1.06 | 1.05 | 0.93 |
| Neutral | G252D | 3326 | 7977.50 | 7049.47 | 1.13 | 1.09 | 0.91 |
| Neutral | G252K | 3329 | 9685.27 | 8502.04 | 1.14 | 1.33 | 1.09 |
| Neutral | G252S | 3336 | 7596.71 | 6986.94 | 1.09 | 1.04 | 0.90 |
| Neutral | G252T | 3334 | 7242.98 | 7147.95 | 1.01 | 0.99 | 0.92 |
| Neutral | G252P | 3344 | 8175.79 | 8226.12 | 0.99 | 1.12 | 1.06 |
| Neutral | G252H | 3328 | 8030.53 | 7802.24 | 1.03 | 1.10 | 1.00 |
| Neutral | G252C | 3331 | 5540.29 | 5421.44 | 1.02 | 0.76 | 0.70 |
| Neutral | G252V | 3341 | 7910.50 | 7997.71 | 0.99 | 1.09 | 1.03 |
| Neutral | G252I | 3340 | 7702.75 | 7964.05 | 0.97 | 1.06 | 1.02 |
| Neutral | P253C | 3350 | 7906.13 | 8213.73 | 0.96 | 0.99 | 1.04 |
| Neutral | P253G | 3356 | 9640.00 | 8446.66 | 1.14 | 1.20 | 1.07 |
| Neutral | P253Q | 3352 | 9482.36 | 8631.24 | 1.10 | 1.18 | 1.09 |
| Neutral | P253I | 3360 | 6906.18 | 7721.21 | 0.89 | 0.86 | 0.97 |
| Neutral | P253L | 3362 | 8851.11 | 8489.29 | 1.04 | 1.10 | 1.07 |
| Neutral | P253R | 3349 | 9020.78 | 8580.86 | 1.05 | 1.12 | 1.08 |
| Neutral | P253A | 3363 | 8697.23 | 8410.29 | 1.03 | 1.08 | 1.06 |
| Neutral | P253E | 3346 | 9074.45 | 8476.99 | 1.07 | 1.13 | 1.07 |
| Neutral | P253Y | 3354 | 7935.28 | 8171.53 | 0.97 | 0.99 | 1.03 |
| Neutral | P253W | 3359 | 6635.85 | 7293.26 | 0.91 | 0.83 | 0.92 |
| Neutral | P253M | 3358 | 6895.66 | 7648.23 | 0.90 | 0.86 | 0.96 |
| Neutral | P253V | 3361 | 7058.87 | 7756.04 | 0.91 | 0.88 | 0.98 |
| Neutral | P253T | 3353 | 6728.25 | 7541.00 | 0.89 | 0.84 | 0.95 |
| Neutral | P253K | 3348 | 6929.49 | 7400.65 | 0.94 | 0.86 | 0.93 |
| Neutral | P253N | 3351 | 7354.73 | 7533.05 | 0.98 | 0.92 | 0.95 |
| Neutral | Q254R | 3368 | 9454.92 | 8474.29 | 1.12 | 1.18 | 1.07 |
| Neutral | Q254G | 3374 | 3549.45 | 3806.63 | 0.93 | 0.44 | 0.48 |
| Neutral | Q254W | 3377 | 3389.45 | 3326.38 | 1.02 | 0.42 | 0.42 |
| Neutral | Q254T | 3371 | 7491.28 | 7853.86 | 0.95 | 0.93 | 0.99 |
| Neutral | Q254A | 3381 | 7226.25 | 7451.70 | 0.97 | 0.90 | 0.94 |
| Neutral | Q254F | 3375 | 6263.95 | 6007.53 | 1.04 | 0.78 | 0.76 |
| Neutral | Q254D | 3364 | 9098.08 | 8154.92 | 1.12 | 1.13 | 1.03 |
| Neutral | Q254P | 3382 | 6827.99 | 7340.40 | 0.93 | 0.85 | 0.93 |
| Neutral | Q254L | 3380 | 7602.15 | 7940.64 | 0.96 | 0.95 | 1.00 |
| Neutral | Q254C | 3369 | 9284.18 | 8479.77 | 1.09 | 1.16 | 1.07 |
| Neutral | Q254Y | 3372 | 8847.02 | 7831.28 | 1.13 | 1.10 | 0.99 |
| Neutral | Q254I | 3378 | 9340.36 | 8662.75 | 1.08 | 1.16 | 1.09 |
| Neutral | Q254E | 3365 | 9466.76 | 8516.08 | 1.11 | 1.18 | 1.07 |
| Neutral | Q254V | 3379 | 9803.92 | 8575.31 | 1.14 | 1.22 | 1.08 |
| Neutral | Q254S | 3373 | 7768.13 | 8801.19 | 0.88 | 1.15 | 1.17 |
| Neutral | T255I | 3397 | 9880.58 | 8415.65 | 1.17 | 1.23 | 1.06 |
| Neutral | T255Q | 3390 | 9537.20 | 8410.86 | 1.13 | 1.19 | 1.06 |
| Neutral | T255P | 3401 | 7468.08 | 7296.37 | 1.02 | 0.93 | 0.92 |
| Neutral | T255R | 3387 | 5740.42 | 4974.50 | 1.15 | 0.72 | 0.63 |
| Neutral | T255C | 3388 | 2626.79 | 2503.21 | 1.05 | 0.33 | 0.32 |
| Neutral | T255N | 3389 | 5128.08 | 4479.75 | 1.14 | 0.64 | 0.57 |
| Neutral | T255S | 3392 | 7334.60 | 6905.71 | 1.06 | 0.91 | 0.87 |
| Neutral | T255V | 3398 | 5463.42 | 5187.78 | 1.05 | 0.68 | 0.65 |
| Neutral | T255E | 3384 | 7691.31 | 7194.23 | 1.07 | 0.96 | 0.91 |
| Neutral | T255G | 3393 | 8166.77 | 7682.14 | 1.06 | 1.02 | 0.97 |
| Neutral | T255K | 3386 | 6636.15 | 5647.18 | 1.18 | 0.83 | 0.71 |
| Neutral | T255A | 3400 | 4436.98 | 4322.98 | 1.03 | 0.55 | 0.55 |
| Neutral | T255F | 3394 | 3562.89 | 3107.64 | 1.15 | 0.44 | 0.39 |
| Neutral | T255L | 3399 | 4904.06 | 4266.71 | 1.15 | 0.61 | 0.54 |
| Neutral | T255H | 3385 | 8243.01 | 7352.60 | 1.12 | 1.22 | 0.98 |
| Up | P256S | 3412 | 10876.81 | 9018.60 | 1.21 | 1.36 | 1.14 |
| Up | P256V | 3418 | 10408.68 | 8594.11 | 1.21 | 1.30 | 1.08 |
| Neutral | P256F | 3414 | 6020.49 | 5181.94 | 1.16 | 0.75 | 0.65 |
| Neutral | P256Y | 3411 | 10270.90 | 8699.77 | 1.18 | 1.28 | 1.10 |
| Neutral | P2561 | 3417 | 9089.54 | 7980.23 | 1.14 | 1.13 | 1.01 |
| Neutral | P256A | 3420 | 9426.67 | 8868.67 | 1.06 | 1.18 | 1.12 |
| Neutral | P256L | 3419 | 8342.08 | 7217.69 | 1.16 | 1.04 | 0.91 |
| Neutral | P256G | 3413 | 4631.84 | 4679.24 | 0.99 | 0.58 | 0.59 |
| Neutral | P256N | 3408 | 4406.75 | 3946.90 | 1.12 | 0.55 | 0.50 |
| Neutral | P256R | 3406 | 4975.17 | 4155.27 | 1.20 | 0.62 | 0.52 |
| Neutral | P256Q | 3409 | 6177.77 | 5546.92 | 1.11 | 0.77 | 0.70 |
| Neutral | P256E | 3403 | 9266.75 | 8366.07 | 1.11 | 1.16 | 1.06 |
| Neutral | P256K | 3405 | 5919.72 | 5928.31 | 1.00 | 0.74 | 0.75 |
| Neutral | P256M | 3415 | 8787.02 | 8554.52 | 1.03 | 1.10 | 1.08 |
| Neutral | P256C | 3407 | 4674.45 | 4633.67 | 1.01 | 0.69 | 0.62 |
| Neutral | K257C | 3425 | 4327.83 | 4267.45 | 1.01 | 0.54 | 0.54 |
| Neutral | K257M | 3433 | 5985.85 | 5236.20 | 1.14 | 0.75 | 0.66 |
| Neutral | K257V | 3436 | 7316.42 | 7115.65 | 1.03 | 0.91 | 0.90 |
| Neutral | K257A | 3438 | 9355.23 | 8528.52 | 1.10 | 1.17 | 1.08 |
| Neutral | K257E | 3422 | 10237.19 | 9141.73 | 1.12 | 1.28 | 1.15 |
| Neutral | K257S | 3430 | 9952.97 | 8464.79 | 1.18 | 1.24 | 1.07 |
| Neutral | K257L | 3437 | 10053.73 | 8711.61 | 1.15 | 1.25 | 1.10 |
| Neutral | K257I | 3435 | 8609.80 | 7806.76 | 1.10 | 1.07 | 0.98 |
| Neutral | K257G | 3431 | 8280.79 | 7718.36 | 1.07 | 1.03 | 0.97 |
| Neutral | K257N | 3426 | 8528.22 | 7707.49 | 1.11 | 1.06 | 0.97 |
| Neutral | K257F | 3432 | 7720.51 | 6633.90 | 1.16 | 0.96 | 0.84 |
| Neutral | K257W | 3434 | 7039.69 | 7120.56 | 0.99 | 0.88 | 0.90 |
| Neutral | K257R | 3424 | 9688.77 | 9114.18 | 1.06 | 1.21 | 1.15 |
| Neutral | K257P | 3439 | 8039.60 | 7464.88 | 1.08 | 1.19 | 0.99 |
| Neutral | K257T | 3428 | 9346.88 | 8849.42 | 1.06 | 1.39 | 1.18 |
| Neutral | A258Q | 3447 | 7000.31 | 6977.33 | 1.00 | 0.87 | 0.88 |
| Neutral | A258Y | 3449 | 6636.02 | 5998.83 | 1.11 | 0.83 | 0.76 |
| Neutral | A258W | 3454 | 9438.05 | 8527.86 | 1.11 | 1.18 | 1.08 |
| Neutral | A258G | 3451 | 7204.05 | 7778.97 | 0.93 | 0.90 | 0.98 |
| Neutral | A258L | 3457 | 1222.62 | 1226.40 | 1.00 | 0.15 | 0.15 |
| Neutral | A258F | 3452 | 9548.91 | 8531.04 | 1.12 | 1.19 | 1.08 |
| Neutral | A258M | 3453 | 8161.79 | 8061.20 | 1.01 | 1.02 | 1.02 |
| Neutral | A258N | 3446 | 7808.83 | 6968.56 | 1.12 | 0.97 | 0.88 |
| Neutral | A258V | 3456 | 8395.80 | 8391.43 | 1.00 | 1.05 | 1.06 |
| Neutral | A258T | 3448 | 8674.71 | 7958.00 | 1.09 | 1.08 | 1.00 |
| Neutral | A258I | 3455 | 8452.43 | 7509.34 | 1.13 | 1.05 | 0.95 |
| Up | A258D | 3440 | 7741.51 | 6346.88 | 1.22 | 0.97 | 0.80 |
| Neutral | A258R | 3444 | 9008.56 | 7908.51 | 1.14 | 1.12 | 1.00 |
| Neutral | A258E | 3441 | 10198.40 | 8709.16 | 1.17 | 1.27 | 1.10 |
| Neutral | A258P | 3458 | 10414.06 | 9178.82 | 1.13 | 1.55 | 1.22 |

The 199 hMMP-1 putative hit mutants were rescreened, using the same assay, and 104 primary hits were confirmed (see Table 11, below). hMMP-1 mutants that were active at 25° C. and had at least a 16% decrease in activity at 37° C. (e.g., the ratio of the activities at 25° C. or 37° C. (25° C./37° C.) is greater than or equal to 1.2) were deemed to be confirmed primary temperature sensitive hits.

Table 10, below, lists the hMMP-1 mutation, the average RFU at 25° C. and 37° C., and the ratio of the activities (25° C./37° C.). The Table also lists the temperature phenotype: DOWN, indicates the ratio (25° C./37° C.) of the activity of the mutant is decreased compared to the ratio (25° C./37° C.) of the activity of the wildtype, i.e. decreased greater than 16% the activity of the wildtype; NEUTRAL, indicates the ratio (25° C./37° C.) of the activity of the mutant is similar to the ratio (25° C./37° C.) of the activity of wildtype, i.e. within 16% of the activity of the wildtype; and UP, indicates the ratio (25° C./37° C.) of the activity of the mutant is increased compared to the ratio (25° C./37° C.) of the activity of the wildtype, i.e. increased more than 16% the activity of the wildtype.

Table 10, below, also lists the residual activities at 25° C. and 37° C., as compared to wild type hMMP-1. The residual activity is the ratio of the hMMP-1 mutant activity versus the wildtype hMMP-1 activity at the indicated temperature, either 25° C. or 37° C. A ratio of less than one indicates that a given mutant has less activity than the wildtype at the indicated temperature and a ratio of greater than one indicates that the mutant has more activity than the wildtype. Several of the hMMP-1 primary hit mutants exhibited activities that were comparable to or greater than wildtype hMMP-1 at 25° C. All of the hMMP-1 confirmed as primary hits exhibited decreated activities at 37° C., thereby reconfirming their decreased activity at elevated temperatures.

TABLE 10
Results of Initial Screen for Temperature Sensitive hMMP-1 mutants

| | | | | | | Res. | |
|---|---|---|---|---|---|---|---|
| Temp. | | | Avg. | Avg. | Ratio | Act. | Res. Act. |
| Pheno- | hMMP-1 | | RFU | RFU | 25° C./ | Mut/wt | Mut/wt |
| type | mutation | SEQ ID NO | 25° C. | 37° C. | 37° C. | 25° C. | 37° C. |
| Neutral | T84F | 847 | 6312.72 | 6453.46 | 0.98 | 1.10 | 1.27 |
| Neutral | E85F | 866 | 6092.47 | 6362.37 | 0.96 | 1.06 | 1.26 |
| Up | L95K | 6 | 1333.28 | 1191.46 | 1.12 | 0.15 | 0.14 |
| Down | L95I | 18 | 1707.98 | 2294.02 | 0.74 | 0.30 | 0.45 |
| Down | R98D | 1083 | 2905.96 | 3867.31 | 0.75 | 0.33 | 0.47 |
| Down | 199Q | 1109 | 3318.21 | 4623.91 | 0.72 | 0.37 | 0.56 |
| Down | E100V | 512 | 3980.72 | 5009.20 | 0.79 | 1.26 | 1.01 |
| Neutral | E100R | 500 | 7410.11 | 7964.52 | 0.93 | 0.83 | 0.96 |
| Neutral | E100S | 506 | 3768.09 | 4664.58 | 0.81 | 0.42 | 0.56 |
| Neutral | E100T | 504 | 6985.28 | 7478.12 | 0.93 | 0.79 | 0.90 |
| Neutral | E100F | 508 | 6709.27 | 7436.60 | 0.90 | 0.75 | 0.90 |
| Neutral | E100L | 511 | 8824.19 | 8458.79 | 1.04 | 0.99 | 1.02 |
| Neutral | E100N | 502 | 8809.68 | 8215.63 | 1.07 | 0.99 | 0.99 |
| Neutral | T103Y | 524 | 1181.09 | 1423.76 | 0.83 | 0.37 | 0.29 |
| Neutral | P104A | 1177 | 8861.30 | 8360.82 | 1.06 | 1.00 | 1.01 |
| Up | P104M | 1172 | 6709.44 | 7118.65 | 0.94 | 0.88 | 0.75 |
| Up | D105A | 39 | 2674.16 | 1227.06 | 2.18 | 0.65 | 0.24 |
| Up | D105E | 33 | 2009.56 | 1221.58 | 1.65 | 0.49 | 0.24 |
| Up | D105G | 32 | 2407.89 | 1686.68 | 1.43 | 0.58 | 0.34 |
| Up | D105I | 36 | 1732.38 | 1105.99 | 1.57 | 0.42 | 0.22 |
| Up | D105L | 38 | 1563.61 | 859.56 | 1.82 | 0.38 | 0.17 |
| Up | D105N | 27 | 3766.72 | 1475.08 | 2.55 | 0.91 | 0.29 |
| Up | D105R | 25 | 3892.02 | 2016.90 | 1.93 | 0.94 | 0.40 |
| Up | D105S | 31 | 3646.49 | 2727.22 | 1.34 | 0.88 | 0.54 |
| Up | D105T | 29 | 2513.64 | 1729.46 | 1.45 | 0.61 | 0.34 |
| Up | D105W | 35 | 2565.93 | 1855.05 | 1.38 | 0.62 | 0.37 |
| Neutral | D105E | 22 | 4000.92 | 3366.64 | 1.19 | 0.59 | 0.45 |
| Neutral | L106C | 1183 | 2995.56 | 3678.33 | 0.81 | 0.34 | 0.44 |
| Neutral | L106S | 1188 | 2730.64 | 2899.36 | 0.94 | 0.31 | 0.35 |
| Neutral | A109H | 1237 | 7206.01 | 7536.96 | 0.96 | 0.81 | 0.91 |
| Neutral | D110A | 1271 | 4179.59 | 5112.44 | 0.82 | 0.47 | 0.62 |
| Neutral | V111R | 1277 | 2401.69 | 2925.16 | 0.82 | 0.27 | 0.35 |
| Neutral | D112S | 1301 | 7203.69 | 7600.93 | 0.95 | 0.81 | 0.92 |
| Neutral | A118T | 1414 | 745.83 | 665.63 | 1.12 | 0.13 | 0.13 |
| Down | S123V | 1516 | 3220.29 | 4504.25 | 0.71 | 0.41 | 0.60 |
| Neutral | N124D | 1520 | 6218.73 | 6620.92 | 0.94 | 0.92 | 0.88 |
| Neutral | T126S | 1567 | 7114.42 | 6856.69 | 1.04 | 1.06 | 0.91 |
| Up | G147P | 1975 | 494.94 | 392.93 | 1.26 | 0.07 | 0.05 |
| Up | R150P | 59 | 2291.14 | 828.28 | 2.77 | 0.31 | 0.12 |
| Neutral | R150V | 56 | 6829.28 | 6604.61 | 1.05 | 1.20 | 1.30 |
| Neutral | R150D | 41 | 7230.41 | 6033.28 | 1.20 | 1.26 | 1.19 |
| Down | R150I | 55 | 3120.05 | 4082.34 | 0.76 | 0.39 | 0.55 |
| Neutral | R150H | 43 | 8281.04 | 8056.17 | 1.03 | 1.05 | 1.08 |
| Up | D151G | 70 | 1073.32 | 733.89 | 1.46 | 0.20 | 0.11 |
| Neutral | N152A | 2031 | 6669.94 | 5660.16 | 1.18 | 1.17 | 1.12 |
| Down | N152S | 2023 | 4607.85 | 8096.31 | 0.57 | 0.58 | 1.08 |
| Neutral | S153T | 543 | 10530.07 | 8798.72 | 1.20 | 1.44 | 1.24 |
| Up | F155L | 95 | 1322.13 | 864.19 | 1.53 | 0.25 | 0.13 |
| Up | F155A | 96 | 1250.93 | 760.12 | 1.65 | 0.23 | 0.11 |
| Up | D156H | 99 | 2722.09 | 2081.55 | 1.31 | 0.51 | 0.31 |
| Up | D156L | 114 | 2548.30 | 1597.53 | 1.60 | 0.48 | 0.24 |
| Up | D156A | 115 | 2679.29 | 1734.45 | 1.54 | 0.50 | 0.26 |
| Up | D156W | 111 | 1575.39 | 1268.36 | 1.24 | 0.30 | 0.19 |
| Up | D156V | 113 | 1400.88 | 766.80 | 1.83 | 0.26 | 0.11 |
| Up | D156K | 100 | 1292.89 | 966.62 | 1.34 | 0.24 | 0.14 |
| Up | D156T | 105 | 2871.09 | 1843.03 | 1.56 | 0.54 | 0.27 |
| Up | D156R | 101 | 2431.23 | 1545.89 | 1.57 | 0.46 | 0.23 |
| Up | D156M | 110 | 817.96 | 502.82 | 1.63 | 0.12 | 0.07 |
| Neutral | P158T | 2080 | 4204.23 | 3507.76 | 1.20 | 0.53 | 0.47 |
| Neutral | P158G | 2083 | 6277.86 | 5496.27 | 1.14 | 0.79 | 0.73 |
| Neutral | P158K | 2075 | 6860.82 | 6680.30 | 1.03 | 0.87 | 0.89 |
| Neutral | P158N | 2078 | 3656.04 | 3874.48 | 0.94 | 0.46 | 0.52 |
| Up | G159V | 132 | 2453.98 | 732.46 | 3.35 | 0.34 | 0.10 |
| Up | G1S9T | 125 | 5059.91 | 1734.12 | 2.92 | 0.69 | 0.24 |
| Up | G159M | 129 | 5905.06 | 4874.00 | 1.21 | 0.75 | 0.65 |
| Neutral | G1591 | 131 | 5725.99 | 5357.20 | 1.07 | 0.72 | 0.72 |
| Neutral | G1S9W | 130 | 6787.40 | 6287.71 | 1.08 | 0.86 | 0.84 |
| Neutral | G159L | 133 | 8231.62 | 7638.64 | 1.08 | 1.04 | 1.02 |
| Neutral | G159C | 122 | 2897.77 | 3053.86 | 0.95 | 0.37 | 0.41 |
| Neutral | P170D | 2281 | 1434.38 | 1462.91 | 0.98 | 0.25 | 0.29 |
| Neutral | P170A | 2299 | 2733.72 | 2793.24 | 0.98 | 0.48 | 0.55 |
| Up | G171P | 572 | 1570.74 | 1204.39 | 1.30 | 0.27 | 0.17 |
| Neutral | G171E | 555 | 1154.96 | 1199.65 | 0.96 | 0.20 | 0.24 |
| Neutral | G171D | 554 | 791.81 | 690.33 | 1.15 | 0.14 | 0.14 |
| Up | A176F | 148 | 10486.82 | 6516.31 | 1.61 | 1.31 | 0.78 |
| Neutral | A176W | 150 | 482.38 | 414.85 | 1.16 | 0.06 | 0.06 |
| Neutral | F178T | 2403 | 560.54 | 487.01 | 1.15 | 0.10 | 0.10 |
| Up | F178L | 2411 | 1788.95 | 1314.38 | 1.36 | 0.31 | 0.26 |
| Up | D179N | 160 | 2433.73 | 812.01 | 3.00 | 0.26 | 0.10 |
| Up | D179V | 170 | 604.63 | 490.35 | 1.23 | 0.11 | 0.10 |
| Up | D179C | 159 | 613.81 | 503.76 | 1.22 | 0.11 | 0.10 |
| Up | E180Y | 182 | 6655.19 | 5379.42 | 1.24 | 0.72 | 0.63 |
| Neutral | E180R | 177 | 6932.51 | 6309.81 | 1.10 | 0.75 | 0.74 |
| Up | E180T | 181 | 3718.16 | 2425.13 | 1.53 | 0.40 | 0.29 |
| Up | E180F | 185 | 7014.78 | 5382.78 | 1.30 | 0.76 | 0.63 |
| Up | E180G | 184 | 5952.65 | 4547.28 | 1.31 | 1.04 | 0.90 |
| Up | E180S | 183 | 5217.80 | 3977.60 | 1.31 | 0.91 | 0.78 |
| Up | E180N | 179 | 6534.65 | 4843.84 | 1.35 | 1.14 | 0.96 |
| Up | E180D | 174 | 7738.70 | 6277.22 | 1.23 | 1.35 | 1.24 |
| Neutral | D181T | 200 | 6867.00 | 6057.09 | 1.13 | 0.74 | 0.71 |
| Up | D181L | 209 | 1727.20 | 1274.09 | 1.36 | 0.19 | 0.15 |
| Up | D181K | 195 | 1087.36 | 696.83 | 1.56 | 0.12 | 0.08 |
| Up | D181C | 197 | 549.29 | 447.40 | 1.23 | 0.10 | 0.09 |
| Up | D181G | 203 | 2764.20 | 2056.56 | 1.34 | 0.48 | 0.41 |
| Up | E182T | 219 | 2995.97 | 1779.42 | 1.68 | 0.32 | 0.21 |
| Up | E182Q | 218 | 1393.28 | 804.84 | 1.73 | 0.15 | 0.09 |
| Up | E182M | 224 | 649.73 | 524.43 | 1.24 | 0.11 | 0.10 |
| Neutral | E182G | 222 | 604.92 | 543.78 | 1.11 | 0.11 | 0.11 |
| Up | R183G | 2424 | 7326.36 | 6021.39 | 1.22 | 1.28 | 1.19 |
| Up | R183S | 2423 | 7896.17 | 6240.74 | 1.27 | 1.38 | 1.23 |
| Up | T185R | 235 | 1728.04 | 851.07 | 2.03 | 0.20 | 0.10 |
| Up | T185Y | 239 | 937.75 | 540.66 | 1.73 | 0.11 | 0.07 |
| Up | T185H | 233 | 1448.0 | 4783.89 | 1.85 | 0.17 | 0.10 |
| Up | T185G | 241 | 3922.30 | 1990.15 | 1.97 | 0.46 | 0.24 |
| Up | T185V | 246 | 1648.14 | 897.66 | 1.84 | 0.19 | 0.11 |
| Up | T185Q | 238 | 1594.81 | 583.93 | 2.73 | 0.19 | 0.07 |
| Up | T185A | 248 | 1599.64 | 711.08 | 2.25 | 0.19 | 0.09 |
| Up | T185E | 232 | 1324.02 | 703.76 | 1.88 | 0.16 | 0.09 |
| Neutral | T185D | 231 | 485.86 | 418.67 | 1.16 | 0.06 | 0.06 |
| p | N187R | 254 | 1042.36 | 709.74 | 1.47 | 0.12 | 0.09 |
| p | N187M | 262 | 1731.67 | 995.07 | 1.74 | 0.20 | 0.12 |
| Neutral | N187W | 263 | 1694.86 | 1425.68 | 1.19 | 0.20 | 0.17 |
| Up | N187F | 261 | 1240.41 | 731.98 | 1.69 | 0.15 | 0.09 |
| Up | N187K | 253 | 2331.93 | 1140.19 | 2.05 | 0.27 | 0.14 |
| Up | N1871 | 264 | 1444.98 | 683.03 | 2.12 | 0.17 | 0.08 |
| Up | N187A | 267 | 4379.80 | 2616.49 | 1.67 | 0.52 | 0.32 |
| Neutral | N187G | 260 | 535.06 | 514.10 | 1.04 | 0.07 | 0.07 |
| Neutral | N187C | 255 | 1804.28 | 1860.67 | 0.97 | 0.23 | 0.25 |
| Neutral | N187H | 252 | 1143.07 | 1071.67 | 1.07 | 0.14 | 0.14 |
| Up | F188V | 2486 | 7116.29 | 5860.00 | 1.21 | 1.24 | 1.16 |
| Neutral | R189N | 2495 | 7842.39 | 6675.36 | 1.17 | 1.37 | 1.32 |
| Neutral | R189T | 2497 | 7610.10 | 6459.94 | 1.18 | 1.33 | 1.27 |
| Neutral | R189Q | 2496 | 7465.37 | 6396.79 | 1.17 | 1.30 | 1.26 |
| Up | E190G | 583 | 5313.99 | 4365.93 | 1.22 | 0.75 | 0.48 |
| Up | E190Y | 581 | 7243.54 | 5742.33 | 1.26 | 1.27 | 1.13 |
| Up | E190D | 573 | 7910.21 | 6468.78 | 1.22 | 1.38 | 1.28 |
| Up | Y191V | 607 | 1553.58 | 1254.11 | 1.24 | 0.19 | 0.14 |
| Up | N192H | 613 | 2274.24 | 1058.80 | 2.15 | 0.32 | 0.12 |
| Up | N192S | 620 | 2043.65 | 1630.74 | 1.25 | 0.29 | 0.18 |
| Up | N192D | 611 | 4213.33 | 2216.40 | 1.90 | 0.59 | 0.24 |
| Up | N192C | 616 | 1310.46 | 987.31 | 1.33 | 0.18 | 0.11 |
| Neutral | H194P | 648 | 5264.79 | 5058.19 | 1.04 | 0.74 | 0.56 |
| Up | R195C | 273 | 4231.32 | 1853.20 | 2.28 | 0.60 | 0.20 |
| Neutral | R195W | 282 | 5099.23 | 4524.84 | 1.13 | 0.72 | 0.50 |
| Neutral | R195L | 285 | 5073.57 | 4520.73 | 1.12 | 0.72 | 0.50 |
| Up | R195G | 279 | 5269.21 | 3025.93 | 1.74 | 0.74 | 0.33 |
| Up | R195Q | 275 | 1958.69 | 1361.83 | 1.44 | 0.28 | 0.15 |
| Up | R195A | 286 | 5605.90 | 3852.81 | 1.46 | 0.79 | 0.42 |
| Up | R195D | 269 | 2724.53 | 1907.81 | 1.43 | 0.38 | 0.21 |
| Up | R19SV | 284 | 1711.48 | 1037.62 | 1.65 | 0.24 | 0.11 |
| Up | A197C | 2552 | 4012.80 | 3140.52 | 1.28 | 0.70 | 0.62 |
| Neutral | A198G | 299 | 2610.82 | 2368.26 | 1.10 | 0.37 | 0.26 |
| Up | A198L | 305 | 1339.94 | 726.74 | 1.84 | 0.19 | 0.08 |
| Up | A198M | 301 | 1384.46 | 999.55 | 1.39 | 0.20 | 0.11 |
| Up | G206A | 324 | 4554.61 | 2702.11 | 1.69 | 0.47 | 0.30 |
| Up | G206S | 317 | 1226.37 | 919.66 | 1.33 | 0.13 | 0.10 |
| Up | L207R | 653 | 3476.88 | 1332.44 | 2.61 | 0.36 | 0.15 |
| Neutral | L207V | 665 | 656.95 | 550.54 | 1.19 | 0.08 | 0.07 |
| Neutral | L2071 | 664 | 645.37 | 550.32 | 1.17 | 0.08 | 0.07 |
| Up | L207G | 660 | 610.01 | 484.35 | 1.26 | 0.08 | 0.06 |
| Neutral | S208R | 2703 | 7639.06 | 6465.10 | 1.18 | 1.34 | 1.28 |
| Up | S208L | 2715 | 7811.78 | 6354.14 | 1.23 | 1.37 | 1.25 |
| Up | S210V | 341 | 1190.35 | 856.63 | 1.39 | 0.29 | 0.17 |
| Neutral | S210A | 343 | 1682.05 | 1546.97 | 1.09 | 0.25 | 0.21 |
| Neutral | T211L | 2753 | 2376.23 | 2102.07 | 1.13 | 0.35 | 0.28 |
| Up | D212G | 678 | 1011.62 | 657.28 | 1.54 | 0.24 | 0.13 |
| Neutral | D212H | 669 | 4696.49 | 4001.41 | 1.17 | 0.70 | 0.53 |
| Up | Y218S | 354 | 3702.49 | 3099.73 | 1.19 | 0.58 | 0.43 |
| Up | F223C | 369 | 3115.11 | 2488.91 | 1.25 | 0.53 | 0.35 |
| Up | F223E | 365 | 7194.34 | 5884.03 | 1.22 | 1.22 | 0.83 |
| Up | F223G | 375 | 3236.56 | 2599.04 | 1.25 | 0.55 | 0.36 |
| Up | F223A | 381 | 5226.86 | 3982.92 | 1.31 | 0.89 | 0.56 |
| Up | F223S | 374 | 6006.80 | 4916.07 | 1.22 | 1.02 | 0.69 |
| Neutral | F223K | 367 | 4021.97 | 3712.91 | 1.08 | 0.60 | 0.49 |
| Neutral | F223M | 376 | 525.66 | 441.29 | 1.19 | 0.08 | 0.06 |
| Up | V227C | 388 | 4040.96 | 3278.65 | 1.23 | 0.68 | 0.46 |
| Up | V227D | 383 | 1190.09 | 731.34 | 1.63 | 0.20 | 0.10 |
| Up | V227E | 384 | 5381.63 | 2605.20 | 2.07 | 0.91 | 0.37 |
| Up | V227L | 399 | 4883.98 | 4000.68 | 1.22 | 0.83 | 0.56 |
| Up | V227S | 393 | 3863.33 | 3131.47 | 1.23 | 0.65 | 0.44 |
| Up | V227W | 397 | 1845.46 | 1374.06 | 1.34 | 0.31 | 0.19 |
| Neutral | V227G | 394 | 1040.74 | 883.01 | 1.18 | 0.15 | 0.12 |
| Up | V227H | 385 | 689.20 | 504.65 | 1.37 | 0.10 | 0.07 |
| Up | V227Q | 390 | 696.97 | 506.11 | 1.38 | 0.10 | 0.07 |
| Neutral | V227R | 387 | 664.31 | 561.06 | 1.18 | 0.10 | 0.07 |
| Up | Q228P | 420 | 2862.74 | 1291.55 | 2.22 | 1.33 | 0.44 |
| Up | L229A | 438 | 2627.78 | 2118.07 | 1.24 | 1.22 | 0.72 |
| Up | L229T | 429 | 3780.54 | 1464.25 | 2.58 | 1.75 | 0.50 |
| Up | L2291 | 436 | 1158.56 | 828.94 | 1.40 | 0.54 | 0.28 |
| Up | A230V | 703 | 5030.94 | 3433.18 | 1.47 | 2.33 | 1.17 |
| Up | D233E | 440 | 2881.17 | 1918.57 | 1.50 | 1.33 | 0.65 |
| Up | 1234A | 476 | 1458.10 | 1018.50 | 1.43 | 0.31 | 0.18 |
| Up | 1234T | 467 | 1451.51 | 1188.67 | 1.22 | 0.31 | 0.21 |
| Up | 1234E | 460 | 1301.06 | 840.09 | 1.55 | 0.27 | 0.15 |
| Up | 1234Q | 466 | 1095.18 | 837.53 | 1.31 | 0.23 | 0.15 |
| Up | 1237L | 475 | 2880.14 | 2240.61 | 1.29 | 0.61 | 0.39 |
| Down | 1237W | 3074 | 4188.38 | 5663.94 | 0.74 | 0.62 | 0.75 |
| Neutral | 1237N | 3066 | 5368.49 | 6271.59 | 0.86 | 0.80 | 0.83 |
| Up | 1240S | 488 | 2033.91 | 1204.66 | 1.69 | 0.32 | 0.15 |
| Neutral | 1240A | 495 | 2099.13 | 1776.41 | 1.18 | 0.23 | 0.23 |
| Up | 1240C | 483 | 970.78 | 650.04 | 1.49 | 0.15 | 0.08 |
| Neutral | 1251S | 3317 | 8445.88 | 7160.96 | 1.18 | 1.07 | 0.96 |
| Neutral | 1251W | 3321 | 7305.95 | 6974.26 | 1.05 | 0.92 | 0.93 |
| Neutral | Q254S | 3373 | 7768.13 | 8801.19 | 0.88 | 1.15 | 1.17 |
| Neutral | T2SSH | 3385 | 8243.01 | 7352.60 | 1.12 | 1.22 | 0.98 |
| Neutral | P256C | 3407 | 4674.45 | 4633.67 | 1.01 | 0.69 | 0.62 |
| Neutral | K257P | 3439 | 8039.60 | 7464.88 | 1.08 | 1.19 | 0.99 |
| Neutral | K257T | 3428 | 9346.88 | 8849.42 | 1.06 | 1.39 | 1.18 |
| Neutral | FA258P | 3458 | 10414.06 | 9178.82 | 1.13 | 1.55 | 1.22 |

TABLE 11
Reconfirmed HITs

| Temperature | hMMP-1 | SEQ ID |
|---|---|---|
| Phenotype | mutation | NO |
| Up | L95K | 6 |
| Down | E100V | 512 |
| Neutral | T103Y | 524 |
| Up | D105A | 39 |
| Up | D105F | 33 |
| Up | D105G | 32 |
| Up | D1051 | 36 |
| Up | D105L | 38 |
| Up | D105N | 27 |
| Up | D105R | 25 |
| Up | D105S | 31 |
| Up | D105T | 29 |
| Up | D105W | 35 |
| Up | R150P | 59 |
| Up | D151G | 70 |
| Neutral | S153T | 543 |
| Up | F155L | 95 |
| Up | F155A | 96 |
| Up | D156H | 99 |
| Up | D156L | 114 |
| Up | D156A | 115 |
| Up | D156W | 111 |
| Up | D156V | 113 |
| Up | D156K | 100 |
| Up | D156T | 105 |
| Up | D156R | 101 |
| Up | G159V | 132 |
| Up | G159T | 125 |
| Up | G171P | 572 |
| Up | A176F | 148 |
| Up | D179N | 160 |
| Up | E180Y | 182 |
| Neutral | E180R | 177 |
| Up | E180T | 181 |
| Up | E180F | 185 |
| Neutral | D181T | 200 |
| Up | D181L | 209 |
| Up | D151K | 195 |
| Up | E182T | 219 |
| Up | E182Q | 218 |
| Up | T185R | 235 |
| Up | T185Y | 239 |
| Up | T185H | 233 |
| Up | T185G | 241 |
| Up | T185V | 246 |
| Up | T185Q | 238 |
| Up | T185A | 248 |
| Up | T185E | 232 |
| Up | N187R | 254 |
| Up | N187M | 262 |
| Neutral | N187W | 263 |
| Up | N187F | 261 |
| Up | N187K | 253 |
| Up | N1871 | 264 |
| Up | N187A | 267 |
| Up | E190G | 583 |
| Up | Y191V | 607 |
| Up | N192H | 613 |
| Up | N192S | 620 |
| Up | N192D | 611 |
| Up | N192C | 616 |
| Neutral | H194P | 648 |
| Up | R195C | 273 |
| Neutral | R195W | 282 |
| Neutral | R195L | 285 |
| Up | R195G | 279 |
| Up | R195Q | 275 |
| Up | R195A | 286 |
| Up | R195D | 269 |
| Up | R195V | 284 |
| Neutral | A198G | 299 |
| Up | A198L | 305 |
| Up | A198M | 301 |
| Up | G206A | 324 |
| Up | G206S | 317 |
| Up | L207R | 653 |
| Up | S210V | 341 |
| Up | D212G | 678 |
| Up | Y218S | 354 |
| Up | F223C | 369 |
| Up | F223E | 365 |
| Up | F223G | 375 |
| Up | F223A | 381 |
| Up | F223S | 374 |
| Up | V227C | 388 |
| Up | V227D | 383 |
| Up | V227E | 384 |
| Up | V227L | 399 |
| Up | V227S | 393 |
| Up | V227W | 397 |
| Up | Q228P | 420 |
| Up | L229A | 438 |
| Up | L229T | 429 |
| Up | L2291 | 436 |
| Up | A230V | 703 |
| Up | D233E | 440 |
| Up | I234A | 476 |
| Up | I234T | 467 |
| Up | I234E | 460 |
| Up | I234Q | 466 |
| Up | I237L | 475 |
| Up | I240S | 488 |
| Neutral | I240A | 495 |
| Up | I240C | 483 |

B. 14-mL Protein Expression

In this example, the hMMP-1 mutants that were identified as temperature sensitive primary hits in Example 2 were expressed in 14 ml culture tubes and their enzymatic activity was measured at 25° C., 34° C. and 37° C. for 1 hour, 2 hours or overnight in order to verify the desired phenotype of decreased activity at elevated temperatures. Protein was expressed and purified as in Example 1 with the exception that the expression was performed in 14 ml tubes rather than a 96-well plate.

Four (4) μl of each hMMP-1 mutant supernatant was transferred to a 96-well microplate. Supernatants were activated with APMA as described in Example 2A above, except that the solution was incubated at the reaction temperature of 25° C., 34° C., or 37° C. for 2 hours. As above, following activation, 100 μl of TCNB containing 10 μM Mca-K-P-L-G-L-Dpa-A-R-NH₂ fluorescent substrate was added to each tube at the indicated reaction temperature (25° C., 34° C. or 37° C.) for one hour. Wildtype hMMP-1 was used as a positive control and supernatant from cells transformed with the vector was used as a negative control. Fluorescence was detected by measuring fluorescence in a fluorescent plate reader at 320 nm exitation/405 nm emission. Relative fluorescence units (RFU) were determined. Duplicate reactions were performed for each sample, reaction temperature, and positive and negative control.

The data is shown in Table 12A (1 hour incubation); Table 12B (2 hour incubations) and Table 12C (overnight incubation), below. Mutants that were active at 25° C. but demonstrated at least a 33% decreased activity at 34° C. or 37° C. (i.e. had a ratio of activity at 25° C. and 34° C. or a ratio of activity of 25° C. and 37° C. equal to or greater than 1.5 under any of the time point conditions tested were identified as temperature sensitive hits. Tables 12A-12C, below, list the hMMP-1 mutation, the RFU at 25° C., 34° C. and 37° C., and the ratio of the activities (at both 25° C./34° C. and 25° C./37° C.) of 64 hMMP-1 mutants whose decreased enzymatic activity at elevated temperatures were confirmed. Some of the hMMP-1 mutants, were noticeably more active at 25° C. than at an elevated temperature. For example, hMMP-1 mutant D179N (SEQ ID NO:160) was 87.5% more active at 25° C. than 37° C. after an overnight incubation (see e.g. Table 12C). Additionally, although expression levels, and therefore overall RFU values, varied in different experiments, the ratios of the activities remained the same. For example, mutant D156T was tested twice (see Table 12C below) and although each test gave different data RFU values the ratio of the values were similar and consistently within the 1.5 ratio parameter.

TABLE 12A
Temperature Sensitive hMMP-1 Mutants, 1 hour incubation

| | | | | | Ratio | Ratio |
|---|---|---|---|---|---|---|
| hMMP-1 | SEQ ID | RFU | RFU | RFU | 25° C./ | 25° C./ |
| mutation | NO | 25° C. | 34° C. | 37° C. | 34° C. | 37° C. |
| L95K | 6 | 2677.64 | 553.00 | 572.70 | 4.84 | 4.68 |
| D105A | 39 | 3496.48 | 697.79 | 1119.92 | 5.01 | 3.12 |
| D105F | 33 | 1749.85 | 554.69 | 685.49 | 3.15 | 2.55 |
| D105G | 32 | 7450.35 | 2196.32 | 3514.50 | 3.39 | 2.12 |
| D105I | 36 | 4720.96 | 638.42 | 943.44 | 7.39 | 5.00 |
| D105L | 38 | 2636.80 | 490.04 | 552.90 | 5.38 | 4.77 |
| D105N | 27 | 7487.95 | 776.33 | 1513.73 | 9.65 | 4.95 |
| D105R | 25 | 1732.70 | 641.23 | 736.92 | 2.70 | 2.35 |
| D105S | 31 | 8637.40 | 3782.36 | 6510.05 | 2.28 | 1.33 |
| D105W | 35 | 4263.51 | 1321.69 | 2422.77 | 3.23 | 1.76 |
| D105ST | 29 | 2666.45 | 770.72 | 1685.33 | 3.46 | 1.58 |
| R150P | 59 | 7568.19 | 1678.59 | 2010.33 | 4.51 | 3.76 |
| D151G | 70 | 973.47 | 517.98 | 595.63 | 1.88 | 1.63 |
| F155A | 96 | 1800.92 | 592.07 | 596.31 | 3.04 | 3.02 |
| D156K | 100 | 8718.91 | 1733.90 | 1839.60 | 5.03 | 4.74 |
| D156T | 105 | 8034.06 | 2216.02 | 2255.25 | 3.63 | 3.56 |
| D156L | 114 | 1825.01 | 528.43 | 619.10 | 3.45 | 2.95 |
| D156A | 115 | 1495.21 | 450.17 | 496.04 | 3.32 | 3.01 |
| D156W | 111 | 1006.97 | 463.48 | 493.84 | 2.17 | 2.04 |
| D156V | 113 | 1140.60 | 484.30 | 504.38 | 2.36 | 2.26 |
| D156T | 105 | 2796.00 | 581.90 | 743.53 | 4.80 | 3.76 |
| D156H | 99 | 3489.60 | 578.59 | 711.59 | 6.03 | 4.90 |
| D156R | 101 | 4983.67 | 678.23 | 734.95 | 7.35 | 6.78 |
| G159V | 132 | 3416.77 | 705.80 | 739.87 | 4.84 | 4.62 |
| G159T | 125 | 4081.99 | 1732.63 | 1865.15 | 2.36 | 2.19 |
| A176F | 148 | 967.31 | 539.31 | 517.16 | 1.79 | 1.87 |
| D179N | 160 | 4105.85 | 492.00 | 513.37 | 8.35 | 8.00 |
| E180Y | 182 | 8803.90 | 3904.31 | 5268.18 | 2.25 | 1.67 |
| E180T | 181 | 5957.38 | 1155.89 | 1430.72 | 5.15 | 4.16 |
| E180F | 185 | 7484.41 | 2677.89 | 3141.69 | 2.79 | 2.38 |
| D181L | 209 | 1629.22 | 559.04 | 549.09 | 2.91 | 2.97 |
| D181K | 195 | 844.40 | 570.98 | 569.44 | 1.48 | 1.48 |
| E182T | 219 | 2244.96 | 653.93 | 668.01 | 3.43 | 3.36 |
| E182Q | 218 | 1066.68 | 583.87 | 582.84 | 1.83 | 1.83 |
| T18SR | 235 | 1599.19 | 867.00 | 872.66 | 1.84 | 1.83 |
| T185H | 233 | 3616.30 | 1601.20 | 1842.01 | 2.26 | 1.96 |
| T18SQ | 238 | 4365.21 | 1512.02 | 1899.46 | 2.89 | 2.30 |
| T185A | 248 | 1374.00 | 567.04 | 608.05 | 2.42 | 2.26 |
| T185E | 232 | 2145.28 | 1263.20 | 1399.76 | 1.70 | 1.53 |
| N187R | 254 | 1659.90 | 955.75 | 1054.91 | 1.74 | 1.57 |
| N187M | 262 | 2842.50 | 1343.95 | 1464.36 | 2.12 | 1.94 |
| N187F | 261 | 1846.10 | 716.62 | 786.07 | 2.58 | 2.35 |
| N187K | 253 | 2428.31 | 1703.73 | 1914.84 | 1.43 | 1.27 |
| N187I | 264 | 2455.44 | 717.51 | 773.59 | 3.42 | 3.17 |
| R195V | 284 | 3121.02 | 1947.80 | 2132.94 | 1.60 | 1.46 |
| A198L | 305 | 4547.61 | 1570.19 | 2061.87 | 2.90 | 2.21 |
| A198M | 301 | 1948.92 | 1101.86 | 1535.22 | 1.77 | 1.27 |
| G206A | 324 | 667.50 | 543.90 | 540.79 | 1.23 | 1.23 |
| G206S | 317 | 608.46 | 427.44 | 412.07 | 1.42 | 1.48 |
| S210V | 341 | 1952.12 | 961.54 | 1791.55 | 2.03 | 1.09 |
| Y218S | 354 | 1674.47 | 1531.03 | 1573.00 | 1.09 | 1.06 |
| F223E | 365 | 5837.16 | 2747.99 | 4955.08 | 2.12 | 1.18 |
| V227C | 388 | 1138.96 | 684.05 | 722.68 | 1.67 | 1.58 |
| V227E | 384 | 5892.76 | 653.81 | 803.12 | 9.01 | 7.34 |
| V227W | 397 | 716.50 | 607.92 | 646.75 | 1.18 | 1.11 |
| Q228P | 420 | 676.11 | 488.99 | 495.88 | 1.38 | 1.36 |
| L229T | 429 | 768.59 | 492.66 | 491.49 | 1.56 | 1.56 |
| L229I | 436 | 1470.04 | 753.87 | 1231.17 | 1.95 | 1.19 |
| D233E | 440 | 1195.07 | 959.25 | 1056.45 | 1.25 | 1.13 |
| 1234A | 476 | 1402.15 | 1014.61 | 1127.63 | 1.38 | 1.24 |
| 1234T | 467 | 857.79 | 644.52 | 712.49 | 1.33 | 1.20 |
| 1234E | 460 | 2281.82 | 591.10 | 762.52 | 3.86 | 2.99 |
| 1240S | 488 | 2678.36 | 776.88 | 1314.40 | 3.45 | 2.04 |
| 1240C | 483 | 1540.91 | 474.82 | 666.63 | 3.25 | 2.31 |

TABLE 12B
Sensitive hMMP-1 Mutants, 2 hours incubation

| | SEQ | | | | Ratio | Ratio |
|---|---|---|---|---|---|---|
| hMMP-1 | ID | RFU | RFU | RFU | 25° C./ | 25° C./ |
| mutation | NO | 25° C. | 34° C. | 37° C. | 34° C. | 37° C. |
| L95K | 6 | 4650.42 | 748.29 | 746.89 | 6.21 | 6.23 |
| D105A | 39 | 5669.31 | 824.07 | 1336.14 | 6.88 | 4.24 |
| D105F | 33 | 2980.00 | 623.89 | 818.63 | 4.78 | 3.64 |
| D105G | 32 | 8821.81 | 2759.24 | 4313.40 | 3.20 | 2.05 |
| D105I | 36 | 6832.34 | 780.32 | 1110.07 | 8.76 | 6.15 |
| D105L | 38 | 4206.38 | 534.24 | 607.46 | 7.87 | 6.92 |
| D105N | 27 | 8920.05 | 918.13 | 1727.44 | 9.72 | 5.16 |
| D105R | 25 | 2821.20 | 722.46 | 813.68 | 3.90 | 3.47 |
| D105S | 31 | 9355.63 | 4607.18 | 7274.97 | 2.03 | 1.29 |
| D105W | 35 | 6663.80 | 1690.93 | 3081.59 | 3.94 | 2.16 |
| D105T | 29 | 4457.16 | 974.63 | 2220.03 | 4.57 | 2.01 |
| R150P | 59 | 8750.30 | 2315.11 | 2497.86 | 3.78 | 3.50 |
| D151G | 70 | 1264.62 | 589.27 | 616.51 | 2.15 | 2.05 |
| F155A | 96 | 2824.01 | 779.72 | 746.59 | 3.62 | 3.78 |
| D156K | 100 | 8576.47 | 2210.63 | 2310.30 | 3.88 | 3.71 |
| D156T | 105 | 8727.27 | 2679.17 | 2752.35 | 3.26 | 3.17 |
| D156L | 114 | 2916.24 | 576.84 | 688.08 | 5.06 | 4.24 |
| D156A | 115 | 2299.63 | 533.68 | 554.21 | 4.31 | 4.15 |
| D156W | 111 | 1502.86 | 539.74 | 575.12 | 2.78 | 2.61 |
| D156V | 113 | 1593.06 | 534.71 | 542.36 | 2.98 | 2.94 |
| D156T | 105 | 4469.68 | 690.87 | 848.14 | 6.47 | 5.27 |
| D156H | 99 | 5387.79 | 698.77 | 819.82 | 7.71 | 6.57 |
| D156R | 101 | 7020.81 | 793.83 | 872.40 | 8.84 | 8.05 |
| G159V | 132 | 4673.44 | 856.78 | 838.46 | 5.45 | 5.57 |
| G159T | 125 | 6704.95 | 2294.40 | 2347.74 | 2.92 | 2.86 |
| A176F | 148 | 1609.85 | 654.43 | 618.72 | 2.46 | 2.60 |
| D179N | 160 | 5660.69 | 644.51 | 656.31 | 8.78 | 8.63 |
| E180Y | 182 | 8557.09 | 4979.24 | 6079.36 | 1.72 | 1.41 |
| E180T | 181 | 7870.99 | 1532.35 | 1794.15 | 5.14 | 4.39 |
| E180F | 185 | 8508.13 | 3597.75 | 3975.22 | 2.36 | 2.14 |
| D181L | 209 | 2710.97 | 619.39 | 611.92 | 4.38 | 4.43 |
| D181K | 195 | 1130.63 | 625.01 | 608.68 | 1.81 | 1.86 |
| E182T | 219 | 3702.08 | 791.23 | 826.28 | 4.68 | 4.48 |
| E182Q | 218 | 1331.50 | 639.84 | 623.11 | 2.08 | 2.14 |
| T185R | 235 | 2637.31 | 1187.63 | 1183.37 | 2.22 | 2.23 |
| T185H | 233 | 5593.77 | 2278.26 | 2534.15 | 2.46 | 2.21 |
| T185Q | 238 | 7006.87 | 2250.58 | 2642.74 | 3.11 | 2.65 |
| T185A | 248 | 2474.96 | 663.82 | 707.09 | 3.73 | 3.50 |
| T185E | 232 | 3948.43 | 2088.15 | 2091.32 | 1.89 | 1.89 |
| N187R | 254 | 3006.08 | 1352.97 | 1421.87 | 2.22 | 2.11 |
| N187M | 262 | 4934.44 | 1811.35 | 1893.07 | 2.72 | 2.61 |
| N187F | 261 | 3227.96 | 877.21 | 931.04 | 3.68 | 3.47 |
| N187K | 253 | 4182.49 | 2425.34 | 2652.79 | 1.72 | 1.58 |
| N187I | 264 | 4218.55 | 849.11 | 887.80 | 4.97 | 4.75 |
| R195V | 284 | 4847.81 | 2724.92 | 2984.10 | 1.78 | 1.62 |
| A198L | 305 | 6756.76 | 2056.50 | 2642.76 | 3.29 | 2.56 |
| A198M | 301 | 3777.50 | 1708.61 | 2155.58 | 2.21 | 1.75 |
| G206A | 324 | 872.27 | 603.01 | 586.57 | 1.45 | 1.49 |
| G206S | 317 | 932.69 | 492.65 | 463.60 | 1.89 | 2.01 |
| S210V | 341 | 3349.95 | 1249.47 | 2314.86 | 2.68 | 1.45 |
| Y218S | 354 | 2878.50 | 2373.98 | 2350.27 | 1.21 | 1.22 |
| F223E | 365 | 8318.70 | 3685.68 | 6209.93 | 2.26 | 1.34 |
| V227C | 388 | 1998.67 | 950.01 | 992.19 | 2.10 | 2.01 |
| V227E | 384 | 7904.54 | 839.00 | 1015.12 | 9.42 | 7.79 |
| V227W | 397 | 996.55 | 729.20 | 787.87 | 1.37 | 1.26 |
| Q228P | 420 | 1082.56 | 607.78 | 586.63 | 1.78 | 1.85 |
| L229T | 429 | 1221.05 | 580.15 | 564.49 | 2.10 | 2.16 |
| L229I | 436 | 2790.27 | 1050.86 | 1803.44 | 2.66 | 1.55 |
| D233E | 440 | 2195.02 | 1393.95 | 1454.71 | 1.57 | 1.51 |
| I234A | 476 | 2375.42 | 1473.70 | 1594.08 | 1.61 | 1.49 |
| I234T | 467 | 1199.18 | 713.83 | 796.81 | 1.68 | 1.50 |
| I234E | 460 | 3920.02 | 705.86 | 923.57 | 5.55 | 4.24 |
| I240S | 488 | 3867.71 | 973.97 | 1575.05 | 3.97 | 2.46 |
| I240C | 483 | 2688.75 | 561.91 | 853.66 | 4.78 | 3.15 |

TABLE 12C
Temperature Sensitive hMMP-1 Mutants, Overnight
incubation

| | SEQ | | | | Ratio | Ratio |
|---|---|---|---|---|---|---|
| hMMP-1 | ID | RFU | RFU | RFU | 25° C./ | 25° C./ |
| mutation | NO | 25° C. | 34° C. | 37° C. | 34° C. | 37° C. |
| L95K | 6 | 7744.34 | 1803.12 | 1677.96 | 4.29 | 4.62 |
| D105A | 39 | 8466.62 | 1302.84 | 1931.17 | 6.50 | 4.38 |
| D105F | 33 | 6725.59 | 938.60 | 1173.23 | 7.17 | 5.73 |
| D105G | 32 | 8940.06 | 3560.75 | 5390.32 | 2.51 | 1.66 |
| D105I | 36 | 8394.32 | 1614.57 | 1958.96 | 5.20 | 4.29 |
| D105L | 38 | 6546.78 | 957.95 | 1070.51 | 6.83 | 6.12 |
| D105N | 27 | 9119.04 | 1459.16 | 2347.74 | 6.25 | 3.88 |
| D105R | 25 | 5775.25 | 1407.06 | 1499.57 | 4.10 | 3.85 |
| D105S | 31 | 9300.85 | 5584.70 | 8234.95 | 1.67 | 1.13 |
| D105W | 35 | 8617.36 | 2851.22 | 4593.06 | 3.02 | 1.88 |
| D105T | 29 | 7910.47 | 1899.25 | 3292.01 | 4.17 | 2.40 |
| R150P | 59 | 9011.11 | 3533.16 | 3559.66 | 2.55 | 2.53 |
| D151G | 70 | 1956.65 | 959.80 | 1097.68 | 2.04 | 1.78 |
| F155A | 96 | 4891.89 | 2016.76 | 1843.31 | 2.43 | 2.65 |
| D156K | 100 | 8696.27 | 3968.92 | 3858.90 | 2.19 | 2.25 |
| D156T | 105 | 8972.20 | 3971.43 | 3854.84 | 2.26 | 2.33 |
| D156L | 114 | 5254.55 | 972.64 | 1232.94 | 5.40 | 4.26 |
| D156A | 115 | 3585.37 | 1098.25 | 1110.73 | 3.26 | 3.23 |
| D156W | 111 | 2570.24 | 1091.27 | 1206.22 | 2.36 | 2.13 |
| D156V | 113 | 2208.99 | 954.21 | 997.64 | 2.31 | 2.21 |
| D156T | 105 | 7229.28 | 1256.02 | 1540.11 | 5.76 | 4.69 |
| D156H | 99 | 7587.19 | 1451.49 | 1763.27 | 5.23 | 4.30 |
| D156R | 101 | 8622.23 | 1735.02 | 1846.71 | 4.97 | 4.67 |
| G159V | 132 | 6555.27 | 1821.53 | 1683.20 | 3.60 | 3.89 |
| G159T | 125 | 9105.95 | 3210.57 | 3160.07 | 2.84 | 2.88 |
| A176F | 148 | 4191.69 | 1414.21 | 1336.32 | 2.96 | 3.14 |
| D179N | 160 | 7317.57 | 1504.84 | 1485.28 | 4.86 | 4.93 |
| E180Y | 182 | 9281.77 | 6080.89 | 6894.61 | 1.53 | 1.35 |
| E180T | 181 | 8475.04 | 2585.89 | 2809.15 | 3.28 | 3.02 |
| E180F | 185 | 9360.74 | 5183.25 | 5335.15 | 1.81 | 1.75 |
| D181L | 209 | 4534.34 | 1078.98 | 1000.80 | 4.20 | 4.53 |
| D181K | 195 | 1869.47 | 946.27 | 928.55 | 1.98 | 2.01 |
| E182T | 219 | 6752.25 | 1483.52 | 1496.55 | 4.55 | 4.51 |
| E182Q | 218 | 2212.75 | 1065.07 | 1035.24 | 2.08 | 2.14 |
| T185R | 235 | 6281.97 | 2425.71 | 2300.61 | 2.59 | 2.73 |
| T185H | 233 | 8531.85 | 3164.69 | 3515.59 | 2.70 | 2.43 |
| T185Q | 238 | 9044.23 | 3639.00 | 4012.93 | 2.49 | 2.25 |
| T185A | 248 | 6156.97 | 1110.68 | 1059.61 | 5.54 | 5.81 |
| T185E | 232 | 8479.18 | 3868.06 | 3892.33 | 2.19 | 2.18 |
| N187R | 254 | 7593.11 | 2415.63 | 2370.01 | 3.14 | 3.20 |
| N187M | 262 | 8605.76 | 2769.52 | 2720.28 | 3.11 | 3.16 |
| N187F | 261 | 7352.85 | 1612.23 | 1704.23 | 4.56 | 4.31 |
| N187K | 253 | 8667.36 | 3458.94 | 3709.62 | 2.51 | 2.34 |
| N187I | 264 | 8306.40 | 1459.25 | 1465.77 | 5.69 | 5.67 |
| R195V | 284 | 8634.05 | 4648.03 | 4960.91 | 1.86 | 1.74 |
| A198L | 305 | 8795.36 | 3469.36 | 4181.78 | 2.54 | 2.10 |
| A198M | 301 | 8352.73 | 3215.69 | 3637.79 | 2.60 | 2.30 |
| G206A | 324 | 2492.53 | 1038.14 | 974.96 | 2.40 | 2.56 |
| G206S | 317 | 2845.84 | 908.82 | 808.42 | 3.13 | 3.52 |
| S210V | 341 | 7104.17 | 2441.96 | 3939.90 | 2.91 | 1.80 |
| Y218S | 354 | 7740.61 | 4057.37 | 4093.29 | 1.91 | 1.89 |
| F223E | 365 | 9650.44 | 4849.58 | 7645.34 | 1.99 | 1.26 |
| V227C | 388 | 5833.84 | 2207.20 | 2432.82 | 2.64 | 2.40 |
| V227E | 384 | 8630.90 | 2283.07 | 2152.81 | 3.78 | 4.01 |
| V227W | 397 | 3070.92 | 1370.13 | 1456.45 | 2.24 | 2.11 |
| Q228P | 420 | 3673.33 | 1162.95 | 1081.32 | 3.16 | 3.40 |
| L229T | 429 | 3543.75 | 1103.34 | 1030.05 | 3.21 | 3.44 |
| L229I | 436 | 7333.92 | 1832.18 | 3268.93 | 4.00 | 2.24 |
| D233E | 440 | 6694.93 | 2570.71 | 2661.43 | 2.60 | 2.52 |
| I234A | 476 | 6250.56 | 3890.90 | 4043.80 | 1.61 | 1.55 |
| I234T | 467 | 3507.08 | 1099.58 | 1228.23 | 3.19 | 2.86 |
| I234E | 460 | 7541.73 | 1365.08 | 1901.96 | 5.52 | 3.97 |
| I240S | 488 | 4376.99 | 2108.15 | 2592.19 | 2.08 | 1.69 |
| I240C | 483 | 6170.51 | 1174.96 | 2223.23 | 5.25 | 2.78 |

Table 13 below depicts the residual activity (the ratio of hMMP-1 mutant RFU/wt hMMP-1 RFU) of the hMMP-1 mutants following overnight incubation with the fluorescent peptide. The activity of mutants at 25° C., 34° C., or 37° C. were compared to the activity of wildtype hMMP-1 at the respective temperatures. At 25° C., five hMMP-1 mutants (E180F, E180Y, D156T, D156K, R150P) were more active than wildtype hMMP-1 as indicated by a residual activity >1. At elevated temperatures, all of the hMMP-1 mutants exhibited an overall decrease in activity when compared to wildtype hMMP-1 at the same temperature, thus confirming the phenotype of the hMMP-1 mutants as temperature sensitive mutants.

TABLE 13
Residual Activity of hMMP-1 Temperature Sensitive
Mutants, Overnight Incubation

| | | Residual | Residual | Residual |
|---|---|---|---|---|
| hMMP-1 | SEQ ID | Activity | Activity | Activity |
| mutation | NO | 25° C. | 34° C. | 37° C. |
| L95K | 6 | 0.80 | 0.20 | 0.20 |
| D105A | 39 | 0.93 | 0.15 | 0.22 |
| D105F | 33 | 0.74 | 0.11 | 0.13 |
| D105G | 32 | 0.99 | 0.42 | 0.60 |
| D1051 | 36 | 0.93 | 0.19 | 0.22 |
| D105L | 38 | 0.72 | 0.11 | 0.12 |
| D105N | 27 | 1.01 | 0.17 | 0.26 |
| D105R | 25 | 0.64 | 0.16 | 0.17 |
| D1055 | 31 | 1.03 | 0.65 | 0.92 |
| D105W | 35 | 0.95 | 0.33 | 0.51 |
| D105T | 29 | 0.87 | 0.22 | 0.37 |
| R150P | 59 | 0.99 | 0.41 | 0.44 |
| D151G | 70 | 0.22 | 0.11 | 0.12 |
| F155A | 96 | 0.51 | 0.22 | 0.22 |
| D156K | 100 | 0.97 | 0.46 | 0.46 |
| D156T | 105 | 1.00 | 0.46 | 0.46 |
| D156H | 99 | 0.84 | 0.17 | 0.20 |
| D156R | 101 | 0.95 | 0.20 | 0.21 |
| G159V | 132 | 0.73 | 0.21 | 0.20 |
| G159T | 125 | 1.00 | 0.37 | 0.39 |
| A176F | 148 | 0.43 | 0.16 | 0.16 |
| D179N | 160 | 0.81 | 0.17 | 0.18 |
| E180Y | 182 | 1.02 | 0.70 | 0.85 |
| E180T | 181 | 0.93 | 0.30 | 0.35 |
| E180F | 185 | 1.03 | 0.60 | 0.66 |
| D181L | 209 | 0.50 | 0.12 | 0.12 |
| D181K | 195 | 0.21 | 0.11 | 0.11 |
| E182T | 219 | 0.74 | 0.17 | 0.18 |
| E182Q | 218 | 0.24 | 0.12 | 0.13 |
| T185R | 235 | 0.69 | 0.28 | 0.28 |
| T185H | 233 | 0.94 | 0.37 | 0.43 |
| T185Q | 238 | 1.00 | 0.42 | 0.49 |
| T185A | 248 | 0.68 | 0.13 | 0.13 |
| T185E | 232 | 0.93 | 0.45 | 0.48 |
| N187R | 254 | 0.84 | 0.28 | 0.29 |
| N187M | 262 | 0.95 | 0.32 | 0.33 |
| N187F | 261 | 0.81 | 0.19 | 0.21 |
| N187K | 253 | 0.95 | 0.40 | 0.46 |
| N187I | 264 | 0.92 | 0.17 | 0.18 |
| R195V | 284 | 0.96 | 0.54 | 0.59 |
| A198L | 305 | 0.98 | 0.40 | 0.49 |
| A198M | 301 | 0.87 | 0.36 | 0.42 |
| G206A | 324 | 0.27 | 0.12 | 0.12 |
| G206S | 317 | 0.31 | 0.10 | 0.10 |
| S210V | 341 | 0.78 | 0.29 | 0.44 |
| Y218S | 354 | 0.85 | 0.47 | 0.50 |
| F223E | 365 | 1.07 | 0.57 | 0.86 |
| V227C | 388 | 0.64 | 0.26 | 0.27 |
| V227E | 384 | 0.95 | 0.27 | 0.24 |
| V227W | 397 | 0.34 | 0.16 | 0.16 |
| Q228P | 420 | 0.38 | 0.13 | 0.13 |
| L229T | 429 | 0.37 | 0.12 | 0.12 |
| L229I | 436 | 0.76 | 0.20 | 0.38 |
| D233E | 440 | 0.69 | 0.28 | 0.31 |
| I234A | 476 | 0.69 | 0.45 | 0.45 |
| I234T | 467 | 0.39 | 0.13 | 0.14 |
| I234E | 460 | 0.83 | 0.16 | 0.21 |
| I240S | 488 | 0.48 | 0.25 | 0.29 |
| I240C | 483 | 0.68 | 0.14 | 0.25 |

C. hMMP-1 Top Mutant Hits

Fourteen (14) positions were identified as top hit positions: 95, 105, 150, 156, 159, 179, 180, 182, 185, 187, 198, 227, 234 and 240. Twenty three (23) hMMP-1 mutants at 14 positions were selected as top hits based on two criteria, including: 1) the ratio of the activities (25° C. to 37° C. and 25° C. to 34° C.); and 2) the activity (in RFUs). All of the mutants listed in Table 14 below had an activity greater than 2000 and a ratio of 25° C. to 37° C. greater than 2. The eleven hits identified with a ** are the hits that ranked high for both the ratio or activities and the activity level, and were used to develop a combinatorial library as described in Example 3.

TABLE 14
Top Hits

| | | | |
|---|---|---|---|
| L9SK** | D1051 | D105N** | D105L |
| D105A | D105G | R150P** | D156R |
| D156H | D156K** | D156T** | G159V** |
| G159T | D179N** | E180T** | E180F |
| E182T | T185Q | N187I | A198L** |
| V227E** | I234E | I240S** | |

### Example 3

### Combinatorial hMMP-1 Variant Libraryanapec

51. Generation

In this example, a combinatorial hMMP-1 variant library was generated from the mutants selected in Example 2C and shown in Table 14 with a double asterix (**) Mutants at positions 182, 185 and 187 were excluded in the generation of the combinatorial library because of the importance of these positions for hMMP-1 catalytic activity. The library was generated to contain every possible combination of amino acid variants for each of the selected mutants. Table 15 depicts all mutant combinations theoretically contained in the library. The theoretical diversity of the library is 1536 mutants, which includes wild type, the 11 single mutants and all possible combinations of the mutants. The positions indicated are with respect to positions corresponding to amino acid residues of hMMP-1 set forth in SEQ ID NO:2. Each row and column indicates one polypeptide containing the noted mutations. For example, 156K 179N 227E, refers to a polypeptide containing three amino acid replacements at positions corresponding to positions set forth in SEQ ID NO:2: D by K at position 156, D by N at position 179 and V by E at position 227. The library was generated and expressed as described in Example 1.

The constructed library (designated CPS library) contained a total of 1238 mutants, including the wildtype and 9 individual hits. The distribution of the number of mutations in the library was determined. The constructed and screened library contained 81% of the maximal diversity.

TABLE 15
Combinatorial Library Mutants

| | | | | | | | | | |
|---|---|---|---|---|---|---|---|---|---|
| 95K | | | | | | | | | |
| 105N | | | | | | | | | |
| 150P | | | | | | | | | |
| 156K | | | | | | | | | |
| 156T | | | | | | | | | |
| 159V | | | | | | | | | |
| 179N | | | | | | | | | |
| 180T | | | | | | | | | |
| 198L | | | | | | | | | |
| 227E | | | | | | | | | |
| 240S | | | | | | | | | |
| 227E | 240S | | | | | | | | |
| 198L | 240S | | | | | | | | |
| 198L | 227E | | | | | | | | |
| 180T | 240S | | | | | | | | |
| 180T | 227E | | | | | | | | |
| 180T | 198L | | | | | | | | |
| 179N | 240S | | | | | | | | |
| 179N | 227E | | | | | | | | |
| 179N | 198L | | | | | | | | |
| 179N | 180T | | | | | | | | |
| 159V | 240S | | | | | | | | |
| 159V | 227E | | | | | | | | |
| 159V | 198L | | | | | | | | |
| 159V | 180T | | | | | | | | |
| 159V | 179N | | | | | | | | |
| 156K | 240S | | | | | | | | |
| 156K | 227E | | | | | | | | |
| 156K | 198L | | | | | | | | |
| 156K | 180T | | | | | | | | |
| 156K | 179N | | | | | | | | |
| 156K | 159V | | | | | | | | |
| 156T | 240S | | | | | | | | |
| 156T | 227E | | | | | | | | |
| 156T | 198L | | | | | | | | |
| 156T | 180T | | | | | | | | |
| 156T | 179N | | | | | | | | |
| 156T | 159V | | | | | | | | |
| 150P | 240S | | | | | | | | |
| 150P | 227E | | | | | | | | |
| 150P | 198L | | | | | | | | |
| 150P | 180T | | | | | | | | |
| 150P | 179N | | | | | | | | |
| 150P | 156K | | | | | | | | |
| 150P | 159V | | | | | | | | |
| 150P | 156T | | | | | | | | |
| 105N | 240S | | | | | | | | |
| 105N | 227E | | | | | | | | |
| 105N | 198L | | | | | | | | |
| 105N | 180T | | | | | | | | |
| 105N | 179N | | | | | | | | |
| 105N | 159V | | | | | | | | |
| 105N | 156K | | | | | | | | |
| 105N | 156T | | | | | | | | |
| 105N | 150P | | | | | | | | |
| 95K | 240S | | | | | | | | |
| 95K | 227E | | | | | | | | |
| 95K | 198L | | | | | | | | |
| 95K | 180T | | | | | | | | |
| 95K | 179N | | | | | | | | |
| 95K | 159V | | | | | | | | |
| 95K | 156T | | | | | | | | |
| 95K | 150P | | | | | | | | |
| 95K | 105N | | | | | | | | |
| 95K | 156K | | | | | | | | |
| 180T | 227E | 240S | | | | | | | |
| 180T | 198L | 240S | | | | | | | |
| 180T | 198L | 227E | | | | | | | |
| 179N | 227E | 240S | | | | | | | |
| 179N | 198L | 240S | | | | | | | |
| 179N | 198L | 227E | | | | | | | |
| 179N | 180T | 240S | | | | | | | |
| 179N | 180T | 227E | | | | | | | |
| 179N | 180T | 198L | | | | | | | |
| 159V | 227E | 240S | | | | | | | |
| 159V | 198L | 240S | | | | | | | |
| 159V | 198L | 227E | | | | | | | |
| 159V | 180T | 240S | | | | | | | |
| 159V | 180T | 227E | | | | | | | |
| 159V | 180T | 198L | | | | | | | |
| 159V | 179N | 240S | | | | | | | |
| 159V | 179N | 227E | | | | | | | |
| 159V | 179N | 198L | | | | | | | |
| 159V | 179N | 180T | | | | | | | |
| 156K | 227E | 240S | | | | | | | |
| 156K | 198L | 240S | | | | | | | |
| 156K | 198L | 227E | | | | | | | |
| 156K | 180T | 240S | | | | | | | |
| 156K | 180T | 227E | | | | | | | |
| 156K | 179N | 240S | | | | | | | |
| 156K | 179N | 227E | | | | | | | |
| 156K | 179N | 198L | | | | | | | |
| 156K | 179N | 180T | | | | | | | |
| 156K | 159V | 240S | | | | | | | |
| 156K | 159V | 227E | | | | | | | |
| 156K | 159V | 198L | | | | | | | |
| 156K | 159V | 180T | | | | | | | |
| 156K | 159V | 179N | | | | | | | |
| 156T | 227E | 240S | | | | | | | |
| 156T | 198L | 240S | | | | | | | |
| 156T | 198L | 227E | | | | | | | |
| 156T | 180T | 240S | | | | | | | |
| 156T | 180T | 227E | | | | | | | |
| 156T | 180T | 198L | | | | | | | |
| 156T | 179N | 240S | | | | | | | |
| 156T | 179N | 227E | | | | | | | |
| 156T | 179N | 198L | | | | | | | |
| 156T | 179N | 180T | | | | | | | |
| 156T | 159V | 240S | | | | | | | |
| 156T | 159V | 227E | | | | | | | |
| 156T | 159V | 198L | | | | | | | |
| 156T | 159V | 180T | | | | | | | |
| 156T | 159V | 179N | | | | | | | |
| 150P | 227E | 240S | | | | | | | |
| 198L | 227E | 240S | | | | | | | |
| 150P | 198L | 240S | | | | | | | |
| 150P | 198L | 227E | | | | | | | |
| 150P | 180T | 240S | | | | | | | |
| 150P | 180T | 227E | | | | | | | |
| 150P | 180T | 198L | | | | | | | |
| 150P | 179N | 240S | | | | | | | |
| 150P | 179N | 227E | | | | | | | |
| 150P | 179N | 198L | | | | | | | |
| 150P | 179N | 180T | | | | | | | |
| 150P | 159V | 240S | | | | | | | |
| 150P | 159V | 227E | | | | | | | |
| 150P | 159V | 198L | | | | | | | |
| 150P | 159V | 180T | | | | | | | |
| 150P | 159V | 179N | | | | | | | |
| 150P | 156K | 240S | | | | | | | |
| 150P | 156K | 227E | | | | | | | |
| 150P | 156K | 198L | | | | | | | |
| 150P | 156K | 180T | | | | | | | |
| 156K | 180T | 198L | | | | | | | |
| 150P | 156K | 159V | | | | | | | |
| 150P | 156T | 240S | | | | | | | |
| 150P | 156T | 227E | | | | | | | |
| 150P | 156T | 198L | | | | | | | |
| 150P | 156T | 180T | | | | | | | |
| 150P | 156T | 179N | | | | | | | |
| 150P | 156T | 159V | | | | | | | |
| 105N | 227E | 240S | | | | | | | |
| 105N | 198L | 240S | | | | | | | |
| 105N | 198L | 227E | | | | | | | |
| 105N | 180T | 240S | | | | | | | |
| 105N | 180T | 227E | | | | | | | |
| 105N | 180T | 198L | | | | | | | |
| 105N | 179N | 240S | | | | | | | |
| 105N | 179N | 227E | | | | | | | |
| 105N | 179N | 198L | | | | | | | |
| 105N | 179N | 180T | | | | | | | |
| 105N | 159V | 240S | | | | | | | |
| 105N | 159V | 227E | | | | | | | |
| 105N | 159V | 198L | | | | | | | |
| 105N | 159V | 180T | | | | | | | |
| 105N | 159V | 179N | | | | | | | |
| 105N | 156K | 240S | | | | | | | |
| 105N | 156K | 227E | | | | | | | |
| 105N | 156K | 198L | | | | | | | |
| 105N | 156K | 180T | | | | | | | |
| 105N | 156K | 179N | | | | | | | |
| 105N | 156K | 159V | | | | | | | |
| 105N | 156T | 240S | | | | | | | |
| 105N | 156T | 227E | | | | | | | |
| 105N | 156T | 198L | | | | | | | |
| 105N | 156T | 180T | | | | | | | |
| 105N | 156T | 179N | | | | | | | |
| 105N | 156T | 159V | | | | | | | |
| 105N | 150P | 240S | | | | | | | |
| 105N | 150P | 227E | | | | | | | |
| 105N | 150P | 198L | | | | | | | |
| 10SN | 150P | 180T | | | | | | | |
| 105N | 150P | 179N | | | | | | | |
| 105N | 150P | 159V | | | | | | | |
| 105N | 150P | 156K | | | | | | | |
| 105N | 150P | 156T | | | | | | | |
| 95K | 227E | 240S | | | | | | | |
| 95K | 198L | 240S | | | | | | | |
| 150P | 156K | 179N | | | | | | | |
| 95K | 180T | 240S | | | | | | | |
| 95K | 180T | 227E | | | | | | | |
| 95K | 180T | 198L | | | | | | | |
| 95K | 179N | 240S | | | | | | | |
| 95K | 179N | 227E | | | | | | | |
| 95K | 179N | 198L | | | | | | | |
| 95K | 179N | 180T | | | | | | | |
| 95K | 159V | 240S | | | | | | | |
| 95K | 159V | 227E | | | | | | | |
| 95K | 159V | 198L | | | | | | | |
| 95K | 159V | 180T | | | | | | | |
| 95K | 159V | 179N | | | | | | | |
| 95K | 156K | 240S | | | | | | | |
| 95K | 156K | 227E | | | | | | | |
| 95K | 156K | 198L | | | | | | | |
| 95K | 156K | 180T | | | | | | | |
| 95K | 156K | 179N | | | | | | | |
| 95K | 156K | 159V | | | | | | | |
| 95K | 198L | 227E | | | | | | | |
| 95K | 156T | 240S | | | | | | | |
| 95K | 156T | 227E | | | | | | | |
| 95K | 156T | 198L | | | | | | | |
| 95K | 156T | 180T | | | | | | | |
| 95K | 156T | 179N | | | | | | | |
| 95K | 156T | 159V | | | | | | | |
| 95K | 150P | 240S | | | | | | | |
| 95K | 150P | 227E | | | | | | | |
| 95K | 150P | 198L | | | | | | | |
| 95K | 150P | 180T | | | | | | | |
| 95K | 150P | 179N | | | | | | | |
| 95K | 150P | 159V | | | | | | | |
| 95K | 150P | 156K | | | | | | | |
| 95K | 150P | 156T | | | | | | | |
| 95K | 105N | 240S | | | | | | | |
| 95K | 10SN | 227E | | | | | | | |
| 95K | 10SN | 198L | | | | | | | |
| 95K | 105N | 180T | | | | | | | |
| 95K | 105N | 179N | | | | | | | |
| 95K | 105N | 159V | | | | | | | |
| 95K | 105N | 156K | | | | | | | |
| 95K | 105N | 156T | | | | | | | |
| 95K | 105N | 150P | | | | | | | |
| 180T | 198L | 227E | 240S | | | | | | |
| 179N | 198L | 227E | 240S | | | | | | |
| 179N | 180T | 227E | 240S | | | | | | |
| 150P | 156T | 180T | 227E | | | | | | |
| 150P | 156T | 180T | 198L | | | | | | |
| 150P | 156T | 179N | 240S | | | | | | |
| 150P | 156T | 179N | 227E | | | | | | |
| 150P | 156T | 179N | 198L | | | | | | |
| 150P | 156T | 179N | 180T | | | | | | |
| 150P | 156T | 159V | 240S | | | | | | |
| 150P | 156T | 159V | 227E | | | | | | |
| 150P | 156T | 159V | 198L | | | | | | |
| 150P | 156T | 159V | 180T | | | | | | |
| 150P | 156T | 159V | 179N | | | | | | |
| 105N | 198L | 227E | 240S | | | | | | |
| 105N | 180T | 227E | 240S | | | | | | |
| 105N | 180T | 198L | 240S | | | | | | |
| 105N | 180T | 198L | 227E | | | | | | |
| 105N | 179N | 227E | 240S | | | | | | |
| 105N | 179N | 198L | 240S | | | | | | |
| 105N | 179N | 198L | 227E | | | | | | |
| 105N | 179N | 180T | 240S | | | | | | |
| 105N | 179N | 180T | 227E | | | | | | |
| 105N | 179N | 180T | 198L | | | | | | |
| 105N | 159V | 227E | 240S | | | | | | |
| 105N | 159V | 198L | 240S | | | | | | |
| 105N | 159V | 198L | 227E | | | | | | |
| 105N | 159V | 180T | 240S | | | | | | |
| 105N | 159V | 180T | 227E | | | | | | |
| 105N | 159V | 180T | 198L | | | | | | |
| 105N | 159V | 179N | 240S | | | | | | |
| 105N | 159V | 179N | 227E | | | | | | |
| 105N | 159V | 179N | 198L | | | | | | |
| 105N | 159V | 179N | 180T | | | | | | |
| 105N | 156K | 227E | 240S | | | | | | |
| 105N | 156K | 198L | 240S | | | | | | |
| 105N | 156K | 198L | 227E | | | | | | |
| 105N | 156K | 180T | 240S | | | | | | |
| 150P | 156K | 159V | 198L | | | | | | |
| 150P | 156K | 159V | 180T | | | | | | |
| 150P | 156K | 159V | 179N | | | | | | |
| 105N | 156K | 159V | 240S | | | | | | |
| 105N | 156K | 159V | 227E | | | | | | |
| 105N | 156K | 159V | 198L | | | | | | |
| 105N | 156K | 159V | 180T | | | | | | |
| 105N | 156K | 159V | 179N | | | | | | |
| 105N | 156T | 227E | 240S | | | | | | |
| 105N | 156T | 198L | 227E | | | | | | |
| 105N | 156T | 180T | 240S | | | | | | |
| 179N | 180T | 198L | 240S | | | | | | |
| 179N | 180T | 198L | 227E | | | | | | |
| 159V | 198L | 227E | 240S | | | | | | |
| 159V | 180T | 227E | 240S | | | | | | |
| 159V | 180T | 198L | 240S | | | | | | |
| 159V | 180T | 198L | 227E | | | | | | |
| 159V | 179N | 227E | 240S | | | | | | |
| 159V | 179N | 198L | 240S | | | | | | |
| 159V | 179N | 198L | 227E | | | | | | |
| 159V | 179N | 180T | 240S | | | | | | |
| 159V | 179N | 180T | 227E | | | | | | |
| 159V | 179N | 180T | 198L | | | | | | |
| 156K | 198L | 227E | 240S | | | | | | |
| 156K | 180T | 227E | 240S | | | | | | |
| 156K | 180T | 198L | 240S | | | | | | |
| 156K | 180T | 198L | 227E | | | | | | |
| 156K | 179N | 227E | 240S | | | | | | |
| 156K | 179N | 198L | 240S | | | | | | |
| 156K | 179N | 198L | 227E | | | | | | |
| 156K | 179N | 180T | 240S | | | | | | |
| 156K | 179N | 180T | 227E | | | | | | |
| 156K | 179N | 180T | 198L | | | | | | |
| 156K | 159V | 227E | 240S | | | | | | |
| 156K | 159V | 198L | 240S | | | | | | |
| 156K | 159V | 198L | 227E | | | | | | |
| 156K | 159V | 180T | 240S | | | | | | |
| 156K | 159V | 180T | 227E | | | | | | |
| 156K | 159V | 180T | 198L | | | | | | |
| 156K | 159V | 179N | 240S | | | | | | |
| 156K | 159V | 179N | 227E | | | | | | |
| 156K | 159V | 179N | 198L | | | | | | |
| 156K | 159V | 179N | 180T | | | | | | |
| 156T | 198L | 227E | 240S | | | | | | |
| 156T | 180T | 227E | 240S | | | | | | |
| 156T | 180T | 198L | 240S | | | | | | |
| 156T | 180T | 198L | 227E | | | | | | |
| 156T | 179N | 227E | 240S | | | | | | |
| 156T | 179N | 198L | 240S | | | | | | |
| 156T | 179N | 198L | 227E | | | | | | |
| 156T | 159V | 227E | 240S | | | | | | |
| 156T | 159V | 198L | 240S | | | | | | |
| 156T | 159V | 198L | 227E | | | | | | |
| 156T | 159V | 180T | 240S | | | | | | |
| 156T | 159V | 180T | 227E | | | | | | |
| 105N | 156T | 198L | 240S | | | | | | |
| 105N | 156T | 180T | 227E | | | | | | |
| 105N | 156T | 180T | 198L | | | | | | |
| 105N | 156T | 179N | 240S | | | | | | |
| 105N | 156T | 179N | 227E | | | | | | |
| 105N | 156T | 179N | 198L | | | | | | |
| 105N | 156T | 179N | 180T | | | | | | |
| 105N | 156T | 159V | 240S | | | | | | |
| 105N | 156T | 159V | 227E | | | | | | |
| 105N | 156T | 159V | 198L | | | | | | |
| 105N | 156T | 159V | 180T | | | | | | |
| 105N | 156T | 159V | 179N | | | | | | |
| 105N | 150P | 227E | 240S | | | | | | |
| 105N | 150P | 198L | 240S | | | | | | |
| 105N | 150P | 198L | 227E | | | | | | |
| 105N | 150P | 180T | 240S | | | | | | |
| 105N | 150P | 180T | 227E | | | | | | |
| 105N | 150P | 180T | 198L | | | | | | |
| 105N | 150P | 179N | 240S | | | | | | |
| 105N | 150P | 179N | 227E | | | | | | |
| 105N | 150P | 179N | 198L | | | | | | |
| 105N | 150P | 179N | 180T | | | | | | |
| 105N | 150P | 159V | 240S | | | | | | |
| 105N | 150P | 159V | 227E | | | | | | |
| 105N | 150P | 159V | 198L | | | | | | |
| 105N | 150P | 159V | 180T | | | | | | |
| 105N | 150P | 159V | 179N | | | | | | |
| 105N | 150P | 156K | 240S | | | | | | |
| 105N | 150P | 156K | 227E | | | | | | |
| 105N | 150P | 156K | 198L | | | | | | |
| 105N | 150P | 156K | 180T | | | | | | |
| 105N | 156K | 180T | 227E | | | | | | |
| 105N | 156K | 180T | 198L | | | | | | |
| 105N | 156K | 179N | 240S | | | | | | |
| 105N | 156K | 179N | 227E | | | | | | |
| 105N | 156K | 179N | 198L | | | | | | |
| 105N | 156K | 179N | 180T | | | | | | |
| 105N | 150P | 156T | 179N | | | | | | |
| 105N | 150P | 156T | 159V | | | | | | |
| 95K | 198L | 227E | 240S | | | | | | |
| 95K | 180T | 227E | 240S | | | | | | |
| 95K | 180T | 198L | 240S | | | | | | |
| 95K | 179N | 227E | 240S | | | | | | |
| 95K | 179N | 198L | 240S | | | | | | |
| 95K | 179N | 198L | 227E | | | | | | |
| 95K | 179N | 180T | 240S | | | | | | |
| 156T | 159V | 180T | 198L | | | | | | |
| 156T | 159V | 179N | 240S | | | | | | |
| 156T | 159V | 179N | 227E | | | | | | |
| 156T | 159V | 179N | 198L | | | | | | |
| 156T | 159V | 179N | 180T | | | | | | |
| 150P | 198L | 227E | 240S | | | | | | |
| 150P | 180T | 227E | 240S | | | | | | |
| 150P | 180T | 198L | 240S | | | | | | |
| 150P | 180T | 198L | 227E | | | | | | |
| 150P | 179N | 227E | 240S | | | | | | |
| 150P | 179N | 198L | 240S | | | | | | |
| 150P | 179N | 198L | 227E | | | | | | |
| 150P | 179N | 180T | 240S | | | | | | |
| 150P | 179N | 180T | 227E | | | | | | |
| 150P | 179N | 180T | 198L | | | | | | |
| 150P | 159V | 227E | 240S | | | | | | |
| 150P | 159V | 198L | 240S | | | | | | |
| 150P | 159V | 198L | 227E | | | | | | |
| 150P | 159V | 180T | 240S | | | | | | |
| 150P | 159V | 180T | 227E | | | | | | |
| 150P | 159V | 180T | 198L | | | | | | |
| 150P | 159V | 179N | 240S | | | | | | |
| 150P | 159V | 179N | 227E | | | | | | |
| 150P | 159V | 179N | 198L | | | | | | |
| 150P | 159V | 179N | 180T | | | | | | |
| 150P | 156K | 227E | 240S | | | | | | |
| 150P | 156K | 198L | 240S | | | | | | |
| 150P | 156K | 198L | 227E | | | | | | |
| 150P | 156K | 180T | 240S | | | | | | |
| 150P | 156K | 180T | 227E | | | | | | |
| 150P | 156K | 180T | 198L | | | | | | |
| 150P | 156K | 179N | 240S | | | | | | |
| 150P | 156K | 179N | 227E | | | | | | |
| 150P | 156K | 179N | 198L | | | | | | |
| 150P | 156K | 179N | 180T | | | | | | |
| 150P | 156K | 159V | 240S | | | | | | |
| 150P | 156K | 159V | 227E | | | | | | |
| 156T | 179N | 180T | 240S | | | | | | |
| 156T | 179N | 180T | 227E | | | | | | |
| 156T | 179N | 180T | 198L | | | | | | |
| 150P | 156T | 227E | 240S | | | | | | |
| 150P | 156T | 198L | 240S | | | | | | |
| 150P | 156T | 198L | 227E | | | | | | |
| 150P | 156T | 180T | 240S | | | | | | |
| 95K | 180T | 198L | 227E | | | | | | |
| 95K | 179N | 180T | 227E | | | | | | |
| 95K | 179N | 180T | 198L | | | | | | |
| 95K | 159V | 227E | 240S | | | | | | |
| 95K | 159V | 198L | 240S | | | | | | |
| 95K | 159V | 198L | 227E | | | | | | |
| 95K | 159V | 180T | 240S | | | | | | |
| 95K | 159V | 180T | 227E | | | | | | |
| 95K | 159V | 180T | 198L | | | | | | |
| 95K | 159V | 179N | 240S | | | | | | |
| 95K | 159V | 179N | 227E | | | | | | |
| 95K | 159V | 179N | 198L | | | | | | |
| 95K | 159V | 179N | 180T | | | | | | |
| 95K | 156K | 227E | 240S | | | | | | |
| 95K | 156K | 198L | 240S | | | | | | |
| 95K | 156K | 198L | 227E | | | | | | |
| 95K | 156K | 180T | 240S | | | | | | |
| 95K | 156K | 180T | 227E | | | | | | |
| 95K | 156K | 180T | 198L | | | | | | |
| 95K | 156K | 179N | 240S | | | | | | |
| 95K | 156K | 179N | 227E | | | | | | |
| 95K | 156K | 179N | 198L | | | | | | |
| 95K | 156K | 179N | 180T | | | | | | |
| 95K | 156K | 159V | 240S | | | | | | |
| 95K | 156K | 159V | 227E | | | | | | |
| 95K | 156K | 159V | 198L | | | | | | |
| 95K | 156K | 159V | 180T | | | | | | |
| 95K | 156K | 159V | 179N | | | | | | |
| 95K | 156T | 227E | 240S | | | | | | |
| 95K | 156T | 198L | 240S | | | | | | |
| 95K | 156T | 198L | 227E | | | | | | |
| 95K | 156T | 180T | 240S | | | | | | |
| 105N | 150P | 156K | 179N | | | | | | |
| 105N | 150P | 156K | 159V | | | | | | |
| 105N | 150P | 156T | 240S | | | | | | |
| 105N | 150P | 156T | 227E | | | | | | |
| 105N | 150P | 156T | 198L | | | | | | |
| 105N | 150P | 156T | 180T | | | | | | |
| 95K | 156T | 179N | 198L | | | | | | |
| 95K | 156T | 179N | 180T | | | | | | |
| 95K | 156T | 159V | 240S | | | | | | |
| 95K | 156T | 159V | 227E | | | | | | |
| 95K | 156T | 159V | 198L | | | | | | |
| 95K | 156T | 159V | 180T | | | | | | |
| 95K | 156T | 159V | 179N | | | | | | |
| 95K | 150P | 227E | 240S | | | | | | |
| 95K | 150P | 198L | 240S | | | | | | |
| 95K | 150P | 198L | 227E | | | | | | |
| 95K | 150P | 180T | 240S | | | | | | |
| 95K | 150P | 180T | 227E | | | | | | |
| 95K | 150P | 180T | 198L | | | | | | |
| 95K | 150P | 179N | 240S | | | | | | |
| 95K | 150P | 179N | 227E | | | | | | |
| 95K | 150P | 179N | 198L | | | | | | |
| 95K | 150P | 179N | 180T | | | | | | |
| 95K | 150P | 159V | 240S | | | | | | |
| 95K | 150P | 159V | 227E | | | | | | |
| 95K | 150P | 159V | 198L | | | | | | |
| 95K | 150P | 159V | 180T | | | | | | |
| 95K | 150P | 159V | 179N | | | | | | |
| 95K | 150P | 156K | 240S | | | | | | |
| 95K | 150P | 156K | 227E | | | | | | |
| 95K | 150P | 156K | 198L | | | | | | |
| 95K | 150P | 156K | 180T | | | | | | |
| 95K | 150P | 156K | 179N | | | | | | |
| 95K | 150P | 156K | 159V | | | | | | |
| 95K | 105N | 156K | 179N | | | | | | |
| 95K | 105N | 156K | 159V | | | | | | |
| 95K | 105N | 156T | 240S | | | | | | |
| 95K | 105N | 156T | 227E | | | | | | |
| 95K | 105N | 156T | 198L | | | | | | |
| 95K | 105N | 156T | 180T | | | | | | |
| 95K | 105N | 156T | 179N | | | | | | |
| 95K | 105N | 156T | 159V | | | | | | |
| 95K | 105N | 156T | 240S | | | | | | |
| 95K | 105N | 150P | 227E | | | | | | |
| 95K | 105N | 150P | 198L | | | | | | |
| 95K | 105N | 150P | 180T | | | | | | |
| 95K | 105N | 150P | 179N | | | | | | |
| 95K | 105N | 150P | 159V | | | | | | |
| 95K | 105N | 150P | 156K | | | | | | |
| 95K | 105N | 150P | 156T | | | | | | |
| 95K | 105N | 179N | 227E | | | | | | |
| 95K | 105N | 179N | 198L | | | | | | |
| 95K | 105N | 179N | 180T | | | | | | |
| 95K | 105N | 159V | 240S | | | | | | |
| 95K | 150P | 156T | 240S | | | | | | |
| 95K | 150P | 156T | 227E | | | | | | |
| 95K | 150P | 156T | 198L | | | | | | |
| 95K | 150P | 156T | 180T | | | | | | |
| 95K | 150P | 156T | 179N | | | | | | |
| 95K | 150P | 156T | 159V | | | | | | |
| 95K | 105N | 227E | 240S | | | | | | |
| 95K | 105N | 198L | 240S | | | | | | |
| 95K | 105N | 198L | 227E | | | | | | |
| 95K | 105N | 180T | 240S | | | | | | |
| 95K | 105N | 180T | 227E | | | | | | |
| 95K | 105N | 180T | 198L | | | | | | |
| 95K | 105N | 179N | 240S | | | | | | |
| 95K | 156T | 180T | 227E | | | | | | |
| 95K | 156T | 180T | 198L | | | | | | |
| 95K | 156T | 179N | 240S | | | | | | |
| 95K | 156T | 179N | 227E | | | | | | |
| 95K | 105N | 159V | 227E | | | | | | |
| 95K | 105N | 159V | 198L | | | | | | |
| 95K | 105N | 159V | 180T | | | | | | |
| 95K | 105N | 159V | 179N | | | | | | |
| 95K | 105N | 156K | 240S | | | | | | |
| 95K | 105N | 156K | 227E | | | | | | |
| 95K | 105N | 156K | 198L | | | | | | |
| 95K | 105N | 156K | 180T | | | | | | |
| 156T | 179N | 180T | 198L | 240S | | | | | |
| 156T | 179N | 180T | 198L | 227E | | | | | |
| 156T | 159V | 198L | 227E | 240S | | | | | |
| 156T | 159V | 180T | 227E | 240S | | | | | |
| 156T | 159V | 180T | 198L | 240S | | | | | |
| 156T | 159V | 180T | 198L | 227E | | | | | |
| 156T | 159V | 179N | 227E | 240S | | | | | |
| 156T | 159V | 179N | 198L | 240S | | | | | |
| 156T | 159V | 179N | 198L | 227E | | | | | |
| 156T | 159V | 179N | 180T | 240S | | | | | |
| 156T | 159V | 179N | 180T | 227E | | | | | |
| 156T | 159V | 179N | 180T | 198L | | | | | |
| 150P | 180T | 198L | 227E | 240S | | | | | |
| 150P | 179N | 198L | 227E | 240S | | | | | |
| 150P | 179N | 180T | 227E | 240S | | | | | |
| 150P | 179N | 180T | 198L | 240S | | | | | |
| 150P | 179N | 180T | 198L | 227E | | | | | |
| 150P | 159V | 198L | 227E | 240S | | | | | |
| 150P | 159V | 180T | 227E | 240S | | | | | |
| 150P | 159V | 180T | 198L | 240S | | | | | |
| 150P | 159V | 179N | 227E | 240S | | | | | |
| 150P | 159V | 179N | 198L | 240S | | | | | |
| 150P | 159V | 179N | 198L | 227E | | | | | |
| 150P | 159V | 179N | 180T | 240S | | | | | |
| 150P | 159V | 179N | 180T | 227E | | | | | |
| 150P | 159V | 179N | 180T | 198L | | | | | |
| 150P | 156K | 198L | 227E | 240S | | | | | |
| 150P | 156K | 180T | 227E | 240S | | | | | |
| 150P | 156K | 180T | 198L | 240S | | | | | |
| 150P | 156K | 180T | 198L | 227E | | | | | |
| 150P | 156K | 179N | 227E | 240S | | | | | |
| 150P | 156K | 179N | 198L | 240S | | | | | |
| 150P | 156K | 179N | 198L | 227E | | | | | |
| 150P | 156K | 179N | 180T | 240S | | | | | |
| 150P | 156K | 179N | 180T | 227E | | | | | |
| 150P | 156K | 179N | 180T | 198L | | | | | |
| 150P | 156K | 159V | 227E | 240S | | | | | |
| 150P | 156K | 159V | 198L | 240S | | | | | |
| 105N | 156K | 180T | 198L | 240S | | | | | |
| 150P | 156K | 159V | 180T | 240S | | | | | |
| 150P | 156K | 159V | 180T | 227E | | | | | |
| 150P | 156K | 159V | 180T | 198L | | | | | |
| 150P | 156K | 159V | 179N | 240S | | | | | |
| 150P | 156K | 159V | 179N | 227E | | | | | |
| 150P | 156K | 159V | 179N | 198L | | | | | |
| 105N | 156K | 179N | 180T | 198L | | | | | |
| 105N | 156K | 159V | 227E | 240S | | | | | |
| 105N | 156K | 159V | 198L | 240S | | | | | |
| 105N | 156K | 159V | 198L | 227E | | | | | |
| 10SN | 156K | 159V | 180T | 240S | | | | | |
| 105N | 156K | 159V | 180T | 227E | | | | | |
| 105N | 156K | 159V | 180T | 198L | | | | | |
| 105N | 156K | 159V | 179N | 240S | | | | | |
| 105N | 156K | 159V | 179N | 227E | | | | | |
| 105N | 156K | 159V | 179N | 198L | | | | | |
| 105N | 156K | 159V | 179N | 180T | | | | | |
| 105N | 156T | 198L | 227E | 240S | | | | | |
| 105N | 156T | 180T | 227E | 240S | | | | | |
| 105N | 156T | 180T | 198L | 240S | | | | | |
| 105N | 156T | 180T | 198L | 227E | | | | | |
| 105N | 156T | 179N | 227E | 240S | | | | | |
| 105N | 156T | 179N | 198L | 240S | | | | | |
| 105N | 156T | 179N | 198L | 227E | | | | | |
| 105N | 156T | 179N | 180T | 240S | | | | | |
| 150P | 156T | 159V | 179N | 198L | | | | | |
| 105N | 156T | 179N | 180T | 198L | | | | | |
| 105N | 156T | 159V | 227E | 240S | | | | | |
| 105N | 156T | 159V | 198L | 240S | | | | | |
| 105N | 156T | 159V | 198L | 227E | | | | | |
| 105N | 156T | 159V | 180T | 240S | | | | | |
| 105N | 156T | 159V | 180T | 227E | | | | | |
| 105N | 156T | 159V | 180T | 198L | | | | | |
| 105N | 156T | 159V | 179N | 240S | | | | | |
| 105N | 156T | 159V | 179N | 227E | | | | | |
| 105N | 156T | 159V | 179N | 198L | | | | | |
| 105N | 156T | 159V | 179N | 180T | | | | | |
| 105N | 150P | 198L | 227E | 240S | | | | | |
| 105N | 150P | 180T | 227E | 240S | | | | | |
| 105N | 150P | 180T | 198L | 240S | | | | | |
| 105N | 150P | 180T | 198L | 227E | | | | | |
| 105N | 150P | 179N | 227E | 240S | | | | | |
| 105N | 150P | 179N | 198L | 240S | | | | | |
| 105N | 150P | 179N | 198L | 227E | | | | | |
| 95K | 179N | 180T | 227E | 240S | | | | | |
| 105N | 150P | 179N | 180T | 227E | | | | | |
| 105N | 150P | 179N | 180T | 198L | | | | | |
| 105N | 150P | 159V | 227E | 240S | | | | | |
| 105N | 150P | 159V | 198L | 240S | | | | | |
| 105N | 150P | 159V | 198L | 227E | | | | | |
| 105N | 150P | 159V | 180T | 240S | | | | | |
| 95K | 159V | 179N | 227E | 240S | | | | | |
| 95K | 159V | 179N | 198L | 240S | | | | | |
| 95K | 159V | 179N | 198L | 227E | | | | | |
| 95K | 159V | 179N | 180T | 240S | | | | | |
| 95K | 159V | 179N | 180T | 227E | | | | | |
| 95K | 159V | 179N | 180T | 198L | | | | | |
| 95K | 156K | 198L | 227E | 240S | | | | | |
| 95K | 156K | 180T | 227E | 240S | | | | | |
| 95K | 156K | 180T | 198L | 240S | | | | | |
| 95K | 156K | 180T | 198L | 227E | | | | | |
| 95K | 156K | 179N | 227E | 240S | | | | | |
| 95K | 156K | 179N | 198L | 240S | | | | | |
| 95K | 156K | 179N | 198L | 227E | | | | | |
| 95K | 156K | 179N | 180T | 240S | | | | | |
| 95K | 156K | 179N | 180T | 227E | | | | | |
| 95K | 156K | 179N | 180T | 198L | | | | | |
| 95K | 156K | 159V | 227E | 240S | | | | | |
| 95K | 156K | 159V | 198L | 240S | | | | | |
| 95K | 156K | 159V | 198L | 227E | | | | | |
| 105N | 150P | 156K | 159V | 180T | | | | | |
| 95K | 156K | 159V | 180T | 227E | | | | | |
| 95K | 156K | 159V | 180T | 198L | | | | | |
| 95K | 156K | 159V | 179N | 240S | | | | | |
| 95K | 156K | 159V | 179N | 227E | | | | | |
| 95K | 156K | 159V | 179N | 198L | | | | | |
| 95K | 156K | 159V | 179N | 180T | | | | | |
| 95K | 156T | 198L | 227E | 240S | | | | | |
| 95K | 156T | 180T | 227E | 240S | | | | | |
| 95K | 156T | 180T | 198L | 240S | | | | | |
| 95K | 156T | 180T | 198L | 227E | | | | | |
| 95K | 156T | 179N | 227E | 240S | | | | | |
| 95K | 156T | 179N | 198L | 240S | | | | | |
| 95K | 156T | 179N | 198L | 227E | | | | | |
| 95K | 156T | 179N | 180T | 240S | | | | | |
| 95K | 156T | 179N | 180T | 227E | | | | | |
| 95K | 156T | 179N | 180T | 198L | | | | | |
| 95K | 156T | 159V | 227E | 240S | | | | | |
| 95K | 156T | 159V | 198L | 240S | | | | | |
| 95K | 150P | 156T | 198L | 227E | | | | | |
| 95K | 156T | 159V | 180T | 240S | | | | | |
| 95K | 156T | 159V | 180T | 227E | | | | | |
| 95K | 156T | 159V | 180T | 198L | | | | | |
| 95K | 156T | 159V | 179N | 240S | | | | | |
| 95K | 156T | 159V | 179N | 227E | | | | | |
| 95K | 156T | 159V | 179N | 198L | | | | | |
| 95K | 150P | 156T | 179N | 180T | | | | | |
| 95K | 150P | 156T | 159V | 240S | | | | | |
| 95K | 150P | 156T | 159V | 227E | | | | | |
| 95K | 150P | 156T | 159V | 198L | | | | | |
| 95K | 150P | 156T | 159V | 180T | | | | | |
| 95K | 150P | 156T | 159V | 179N | | | | | |
| 95K | 105N | 198L | 227E | 240S | | | | | |
| 95K | 105N | 180T | 227E | 240S | | | | | |
| 95K | 105N | 180T | 198L | 240S | | | | | |
| 95K | 105N | 180T | 198L | 227E | | | | | |
| 95K | 105N | 179N | 227E | 240S | | | | | |
| 95K | 105N | 179N | 198L | 240S | | | | | |
| 95K | 105N | 179N | 198L | 227E | | | | | |
| 95K | 105N | 179N | 180T | 240S | | | | | |
| 95K | 105N | 179N | 180T | 227E | | | | | |
| 95K | 105N | 179N | 180T | 198L | | | | | |
| 95K | 105N | 159V | 227E | 240S | | | | | |
| 95K | 105N | 159V | 198L | 240S | | | | | |
| 95K | 105N | 159V | 198L | 227E | | | | | |
| 95K | 150P | 159V | 179N | 198L | | | | | |
| 95K | 105N | 159V | 180T | 227E | | | | | |
| 95K | 105N | 159V | 180T | 198L | | | | | |
| 95K | 105N | 159V | 179N | 240S | | | | | |
| 95K | 105N | 159V | 179N | 227E | | | | | |
| 95K | 105N | 159V | 179N | 198L | | | | | |
| 95K | 105N | 159V | 179N | 180T | | | | | |
| 95K | 105N | 156K | 227E | 240S | | | | | |
| 95K | 105N | 156K | 198L | 240S | | | | | |
| 95K | 105N | 156K | 198L | 227E | | | | | |
| 95K | 105N | 156K | 180T | 240S | | | | | |
| 95K | 105N | 156K | 180T | 227E | | | | | |
| 95K | 105N | 156K | 180T | 198L | | | | | |
| 95K | 105N | 156K | 179N | 240S | | | | | |
| 95K | 105N | 156K | 179N | 227E | | | | | |
| 95K | 105N | 156K | 179N | 198L | | | | | |
| 95K | 105N | 156K | 179N | 180T | | | | | |
| 95K | 105N | 156K | 159V | 240S | | | | | |
| 95K | 105N | 156K | 159V | 227E | | | | | |
| 156K | 159V | 179N | 198L | 227E | | | | | |
| 179N | 180T | 198L | 227E | 240S | | | | | |
| 159V | 180T | 198L | 227E | 240S | | | | | |
| 159V | 179N | 198L | 227E | 240S | | | | | |
| 159V | 179N | 180T | 227E | 240S | | | | | |
| 159V | 179N | 180T | 198L | 240S | | | | | |
| 159V | 179N | 180T | 198L | 227E | | | | | |
| 156K | 180T | 198L | 227E | 240S | | | | | |
| 156K | 179N | 198L | 227E | 240S | | | | | |
| 156K | 179N | 180T | 227E | 240S | | | | | |
| 156K | 179N | 180T | 198L | 240S | | | | | |
| 156K | 179N | 180T | 198L | 227E | | | | | |
| 156K | 159V | 198L | 227E | 240S | | | | | |
| 156K | 159V | 180T | 227E | 240S | | | | | |
| 156K | 159V | 180T | 198L | 240S | | | | | |
| 156K | 159V | 180T | 198L | 227E | | | | | |
| 156K | 159V | 179N | 227E | 240S | | | | | |
| 156K | 159V | 179N | 198L | 240S | | | | | |
| 150P | 156K | 159V | 198L | 227E | | | | | |
| 156K | 159V | 179N | 180T | 240S | | | | | |
| 156K | 159V | 179N | 180T | 227E | | | | | |
| 156K | 159V | 179N | 180T | 198L | | | | | |
| 156T | 180T | 198L | 227E | 240S | | | | | |
| 156T | 179N | 198L | 227E | 240S | | | | | |
| 156T | 179N | 180T | 227E | 240S | | | | | |
| 150P | 156K | 159V | 179N | 180T | | | | | |
| 150P | 156T | 198L | 227E | 240S | | | | | |
| 150P | 156T | 180T | 227E | 240S | | | | | |
| 150P | 156T | 180T | 198L | 240S | | | | | |
| 150P | 156T | 180T | 198L | 227E | | | | | |
| 150P | 156T | 179N | 227E | 240S | | | | | |
| 150P | 156T | 179N | 198L | 240S | | | | | |
| 150P | 156T | 179N | 198L | 227E | | | | | |
| 150P | 156T | 179N | 180T | 240S | | | | | |
| 150P | 156T | 179N | 180T | 227E | | | | | |
| 150P | 156T | 179N | 180T | 198L | | | | | |
| 150P | 156T | 159V | 227E | 240S | | | | | |
| 150P | 156T | 159V | 198L | 240S | | | | | |
| 150P | 156T | 159V | 198L | 227E | | | | | |
| 150P | 156T | 159V | 180T | 240S | | | | | |
| 150P | 156T | 159V | 180T | 227E | | | | | |
| 150P | 156T | 159V | 180T | 198L | | | | | |
| 150P | 156T | 159V | 179N | 240S | | | | | |
| 150P | 156T | 159V | 179N | 227E | | | | | |
| 150P | 159V | 180T | 198L | 227E | | | | | |
| 150P | 156T | 159V | 179N | 180T | | | | | |
| 105N | 180T | 198L | 227E | 240S | | | | | |
| 105N | 179N | 198L | 227E | 240S | | | | | |
| 105N | 179N | 180T | 227E | 240S | | | | | |
| 105N | 179N | 180T | 198L | 240S | | | | | |
| 105N | 179N | 180T | 198L | 227E | | | | | |
| 105N | 159V | 198L | 227E | 240S | | | | | |
| 105N | 159V | 180T | 227E | 240S | | | | | |
| 105N | 159V | 180T | 198L | 240S | | | | | |
| 105N | 159V | 180T | 198L | 227E | | | | | |
| 105N | 159V | 179N | 227E | 240S | | | | | |
| 105N | 159V | 179N | 198L | 240S | | | | | |
| 105N | 159V | 179N | 198L | 227E | | | | | |
| 105N | 159V | 179N | 180T | 240S | | | | | |
| 105N | 159V | 179N | 180T | 227E | | | | | |
| 105N | 159V | 179N | 180T | 198L | | | | | |
| 105N | 156K | 198L | 227E | 240S | | | | | |
| 105N | 156K | 180T | 227E | 240S | | | | | |
| 105N | 150P | 179N | 180T | 240S | | | | | |
| 105N | 156K | 180T | 198L | 227E | | | | | |
| 105N | 156K | 179N | 227E | 240S | | | | | |
| 105N | 156K | 179N | 198L | 240S | | | | | |
| 105N | 156K | 179N | 198L | 227E | | | | | |
| 105N | 156K | 179N | 180T | 240S | | | | | |
| 105N | 156K | 179N | 180T | 227E | | | | | |
| 105N | 150P | 159V | 180T | 227E | | | | | |
| 105N | 150P | 159V | 180T | 198L | | | | | |
| 105N | 150P | 159V | 179N | 240S | | | | | |
| 105N | 150P | 159V | 179N | 227E | | | | | |
| 105N | 150P | 159V | 179N | 198L | | | | | |
| 105N | 150P | 159V | 179N | 180T | | | | | |
| 105N | 150P | 156K | 227E | 240S | | | | | |
| 105N | 150P | 156K | 198L | 240S | | | | | |
| 105N | 150P | 156K | 198L | 227E | | | | | |
| 105N | 150P | 156K | 180T | 240S | | | | | |
| 105N | 150P | 156K | 180T | 227E | | | | | |
| 105N | 150P | 156K | 180T | 198L | | | | | |
| 105N | 150P | 156K | 179N | 240S | | | | | |
| 105N | 150P | 156K | 179N | 227E | | | | | |
| 105N | 150P | 156K | 179N | 198L | | | | | |
| 105N | 150P | 156K | 179N | 180T | | | | | |
| 105N | 150P | 156K | 159V | 240S | | | | | |
| 105N | 150P | 156K | 159V | 227E | | | | | |
| 105N | 150P | 156K | 159V | 198L | | | | | |
| 105N | 156T | 179N | 180T | 227E | | | | | |
| 105N | 150P | 156K | 159V | 179N | | | | | |
| 105N | 150P | 156T | 227E | 240S | | | | | |
| 105N | 150P | 156T | 198L | 240S | | | | | |
| 105N | 150P | 156T | 198L | 227E | | | | | |
| 105N | 150P | 156T | 180T | 240S | | | | | |
| 105N | 150P | 156T | 180T | 227E | | | | | |
| 105N | 150P | 156T | 180T | 198L | | | | | |
| 105N | 150P | 156T | 179N | 240S | | | | | |
| 105N | 150P | 156T | 179N | 227E | | | | | |
| 105N | 150P | 156T | 179N | 198L | | | | | |
| 105N | 150P | 156T | 179N | 180T | | | | | |
| 105N | 150P | 156T | 159V | 240S | | | | | |
| 105N | 150P | 156T | 159V | 227E | | | | | |
| 105N | 150P | 156T | 159V | 198L | | | | | |
| 105N | 150P | 156T | 159V | 180T | | | | | |
| 105N | 150P | 156T | 159V | 179N | | | | | |
| 95K | 180T | 198L | 227E | 240S | | | | | |
| 95K | 179N | 198L | 227E | 240S | | | | | |
| 95K | 156T | 159V | 198L | 227E | | | | | |
| 95K | 179N | 180T | 198L | 240S | | | | | |
| 95K | 179N | 180T | 198L | 227E | | | | | |
| 95K | 159V | 198L | 227E | 240S | | | | | |
| 95K | 159V | 180T | 227E | 240S | | | | | |
| 95K | 159V | 180T | 198L | 240S | | | | | |
| 95K | 159V | 180T | 198L | 227E | | | | | |
| 95K | 156T | 159V | 179N | 180T | | | | | |
| 95K | 150P | 198L | 227E | 240S | | | | | |
| 95K | 150P | 180T | 227E | 240S | | | | | |
| 95K | 150P | 180T | 198L | 240S | | | | | |
| 95K | 150P | 180T | 198L | 227E | | | | | |
| 95K | 150P | 179N | 227E | 240S | | | | | |
| 95K | 150P | 179N | 198L | 240S | | | | | |
| 95K | 150P | 179N | 198L | 227E | | | | | |
| 95K | 150P | 179N | 180T | 240S | | | | | |
| 95K | 150P | 179N | 180T | 227E | | | | | |
| 95K | 150P | 179N | 180T | 198L | | | | | |
| 95K | 150P | 159V | 227E | 240S | | | | | |
| 95K | 150P | 159V | 198L | 240S | | | | | |
| 95K | 150P | 159V | 198L | 227E | | | | | |
| 95K | 150P | 159V | 180T | 240S | | | | | |
| 95K | 150P | 159V | 180T | 227E | | | | | |
| 95K | 150P | 159V | 180T | 198L | | | | | |
| 95K | 150P | 159V | 179N | 240S | | | | | |
| 95K | 150P | 159V | 179N | 227E | | | | | |
| 95K | 156K | 159V | 180T | 240S | | | | | |
| 95K | 150P | 159V | 179N | 180T | | | | | |
| 95K | 150P | 156K | 227E | 240S | | | | | |
| 95K | 150P | 156K | 198L | 240S | | | | | |
| 95K | 150P | 156K | 198L | 227E | | | | | |
| 95K | 150P | 156K | 180T | 240S | | | | | |
| 95K | 150P | 156K | 180T | 227E | | | | | |
| 95K | 150P | 156K | 180T | 198L | | | | | |
| 95K | 150P | 156K | 179N | 240S | | | | | |
| 95K | 150P | 156K | 179N | 227E | | | | | |
| 95K | 150P | 156K | 179N | 198L | | | | | |
| 95K | 150P | 156K | 179N | 180T | | | | | |
| 95K | 150P | 156K | 159V | 240S | | | | | |
| 95K | 150P | 156K | 159V | 227E | | | | | |
| 95K | 150P | 156K | 159V | 198L | | | | | |
| 95K | 150P | 156K | 159V | 180T | | | | | |
| 95K | 150P | 156K | 159V | 179N | | | | | |
| 95K | 150P | 156T | 227E | 240S | | | | | |
| 95K | 150P | 156T | 198L | 240S | | | | | |
| 95K | 105N | 156K | 159V | 198L | | | | | |
| 95K | 150P | 156T | 180T | 240S | | | | | |
| 95K | 150P | 156T | 180T | 227E | | | | | |
| 95K | 150P | 156T | 180T | 198L | | | | | |
| 95K | 150P | 156T | 179N | 240S | | | | | |
| 95K | 150P | 156T | 179N | 227E | | | | | |
| 95K | 150P | 156T | 179N | 198L | | | | | |
| 95K | 105N | 156T | 180T | 227E | | | | | |
| 95K | 105N | 156T | 180T | 198L | | | | | |
| 95K | 105N | 156T | 179N | 240S | | | | | |
| 95K | 105N | 156T | 179N | 227E | | | | | |
| 95K | 105N | 156T | 179N | 198L | | | | | |
| 95K | 105N | 156T | 179N | 180T | | | | | |
| 95K | 105N | 156T | 159V | 240S | | | | | |
| 95K | 105N | 156T | 159V | 227E | | | | | |
| 95K | 105N | 156T | 159V | 198L | | | | | |
| 95K | 105N | 156T | 159V | 180T | | | | | |
| 95K | 105N | 156T | 159V | 179N | | | | | |
| 95K | 105N | 150P | 227E | 240S | | | | | |
| 95K | 105N | 150P | 198L | 240S | | | | | |
| 95K | 105N | 150P | 198L | 227E | | | | | |
| 95K | 105N | 150P | 180T | 240S | | | | | |
| 95K | 105N | 150P | 180T | 227E | | | | | |
| 95K | 105N | 150P | 180T | 198L | | | | | |
| 95K | 105N | 150P | 179N | 240S | | | | | |
| 95K | 105N | 150P | 179N | 227E | | | | | |
| 95K | 105N | 159V | 180T | 240S | | | | | |
| 95K | 105N | 150P | 179N | 180T | | | | | |
| 95K | 105N | 150P | 159V | 240S | | | | | |
| 95K | 105N | 150P | 159V | 227E | | | | | |
| 95K | 105N | 150P | 159V | 198L | | | | | |
| 95K | 105N | 150P | 159V | 180T | | | | | |
| 95K | 105N | 150P | 159V | 179N | | | | | |
| 95K | 105N | 150P | 156K | 240S | | | | | |
| 95K | 105N | 150P | 156K | 227E | | | | | |
| 95K | 105N | 150P | 156K | 198L | | | | | |
| 95K | 105N | 150P | 156K | 180T | | | | | |
| 95K | 105N | 150P | 156K | 179N | | | | | |
| 95K | 105N | 150P | 156K | 159V | | | | | |
| 95K | 105N | 150P | 156T | 240S | | | | | |
| 95K | 105N | 150P | 156T | 227E | | | | | |
| 95K | 105N | 150P | 156T | 198L | | | | | |
| 95K | 105N | 150P | 156T | 180T | | | | | |
| 95K | 105N | 150P | 156T | 179N | | | | | |
| 95K | 105N | 150P | 156T | 159V | | | | | |
| 95K | 105N | 150P | 179N | 198L | | | | | |
| 95K | 105N | 156K | 159V | 180T | | | | | |
| 95K | 105N | 156K | 159V | 179N | | | | | |
| 95K | 105N | 156T | 227E | 240S | | | | | |
| 95K | 105N | 156T | 198L | 240S | | | | | |
| 95K | 105N | 156T | 198L | 227E | | | | | |
| 95K | 105N | 156T | 180T | 240S | | | | | |
| 150P | 156T | 159V | 179N | 198L | 227E | | | | |
| 150P | 156T | 159V | 179N | 180T | 240S | | | | |
| 150P | 156T | 159V | 179N | 180T | 227E | | | | |
| 150P | 156T | 159V | 179N | 180T | 198L | | | | |
| 105N | 179N | 180T | 198L | 227E | 240S | | | | |
| 105N | 159V | 180T | 198L | 227E | 240S | | | | |
| 156K | 159V | 179N | 180T | 198L | 227E | | | | |
| 156T | 179N | 180T | 198L | 227E | 240S | | | | |
| 156T | 159V | 180T | 198L | 227E | 240S | | | | |
| 156T | 159V | 179N | 198L | 227E | 240S | | | | |
| 156T | 159V | 179N | 180T | 227E | 240S | | | | |
| 156T | 159V | 179N | 180T | 198L | 240S | | | | |
| 156T | 159V | 179N | 180T | 198L | 227E | | | | |
| 150P | 179N | 180T | 198L | 227E | 240S | | | | |
| 150P | 159V | 180T | 198L | 227E | 240S | | | | |
| 150P | 159V | 179N | 198L | 227E | 240S | | | | |
| 150P | 159V | 179N | 180T | 227E | 240S | | | | |
| 150P | 159V | 179N | 180T | 198L | 240S | | | | |
| 150P | 159V | 179N | 180T | 198L | 227E | | | | |
| 150P | 156K | 180T | 198L | 227E | 240S | | | | |
| 150P | 156K | 179N | 198L | 227E | 240S | | | | |
| 150P | 156K | 179N | 180T | 227E | 240S | | | | |
| 150P | 156K | 179N | 180T | 198L | 240S | | | | |
| 150P | 156K | 179N | 180T | 198L | 227E | | | | |
| 150P | 156K | 159V | 198L | 227E | 240S | | | | |
| 150P | 156K | 159V | 180T | 227E | 240S | | | | |
| 150P | 156K | 159V | 180T | 198L | 240S | | | | |
| 150P | 156K | 159V | 180T | 198L | 227E | | | | |
| 150P | 156K | 159V | 179N | 227E | 240S | | | | |
| 150P | 156K | 159V | 179N | 198L | 240S | | | | |
| 150P | 156K | 159V | 179N | 198L | 227E | | | | |
| 150P | 156K | 159V | 179N | 180T | 240S | | | | |
| 150P | 156K | 159V | 179N | 180T | 227E | | | | |
| 150P | 156K | 159V | 179N | 180T | 198L | | | | |
| 150P | 156T | 180T | 198L | 227E | 240S | | | | |
| 150P | 156T | 179N | 198L | 227E | 240S | | | | |
| 150P | 156T | 179N | 180T | 227E | 240S | | | | |
| 150P | 156T | 179N | 180T | 198L | 240S | | | | |
| 150P | 156T | 179N | 180T | 198L | 227E | | | | |
| 150P | 156T | 159V | 198L | 227E | 240S | | | | |
| 150P | 156T | 159V | 180T | 227E | 240S | | | | |
| 150P | 156T | 159V | 180T | 198L | 240S | | | | |
| 150P | 156T | 159V | 180T | 198L | 227E | | | | |
| 150P | 156T | 159V | 179N | 227E | 240S | | | | |
| 105N | 150P | 179N | 180T | 198L | 227E | | | | |
| 105N | 150P | 156K | 198L | 227E | 240S | | | | |
| 105N | 150P | 156K | 180T | 227E | 240S | | | | |
| 105N | 150P | 156K | 180T | 198L | 240S | | | | |
| 105N | 150P | 156K | 180T | 198L | 227E | | | | |
| 105N | 150P | 156K | 179N | 227E | 240S | | | | |
| 105N | 150P | 156K | 179N | 198L | 240S | | | | |
| 105N | 150P | 156K | 179N | 198L | 227E | | | | |
| 105N | 150P | 156K | 179N | 180T | 240S | | | | |
| 105N | 150P | 156K | 179N | 180T | 227E | | | | |
| 105N | 150P | 156K | 179N | 180T | 198L | | | | |
| 105N | 150P | 156K | 159V | 227E | 240S | | | | |
| 105N | 150P | 156K | 159V | 198L | 240S | | | | |
| 105N | 150P | 156K | 159V | 198L | 227E | | | | |
| 105N | 150P | 156K | 159V | 180T | 240S | | | | |
| 105N | 150P | 156K | 159V | 180T | 227E | | | | |
| 105N | 150P | 156K | 159V | 180T | 198L | | | | |
| 105N | 150P | 156K | 159V | 179N | 240S | | | | |
| 105N | 150P | 156K | 159V | 179N | 227E | | | | |
| 105N | 150P | 156K | 159V | 179N | 198L | | | | |
| 105N | 150P | 156K | 159V | 179N | 180T | | | | |
| 105N | 150P | 159V | 198L | 227E | 240S | | | | |
| 105N | 150P | 159V | 180T | 227E | 240S | | | | |
| 105N | 150P | 159V | 180T | 198L | 240S | | | | |
| 105N | 150P | 159V | 180T | 198L | 227E | | | | |
| 105N | 150P | 156T | 198L | 227E | 240S | | | | |
| 105N | 150P | 156T | 180T | 227E | 240S | | | | |
| 105N | 150P | 156T | 180T | 198L | 240S | | | | |
| 105N | 150P | 156T | 180T | 198L | 227E | | | | |
| 105N | 150P | 156T | 179N | 227E | 240S | | | | |
| 105N | 150P | 156T | 179N | 198L | 240S | | | | |
| 105N | 150P | 156T | 179N | 198L | 227E | | | | |
| 105N | 150P | 156T | 179N | 180T | 240S | | | | |
| 105N | 150P | 156T | 179N | 180T | 227E | | | | |
| 105N | 150P | 156T | 179N | 180T | 198L | | | | |
| 105N | 150P | 156T | 159V | 227E | 240S | | | | |
| 105N | 150P | 156T | 159V | 198L | 240S | | | | |
| 105N | 150P | 156T | 159V | 198L | 227E | | | | |
| 105N | 150P | 156T | 159V | 180T | 240S | | | | |
| 95K | 150P | 179N | 180T | 198L | 240S | | | | |
| 105N | 150P | 156T | 159V | 180T | 198L | | | | |
| 105N | 150P | 156T | 159V | 179N | 240S | | | | |
| 105N | 150P | 156T | 159V | 179N | 227E | | | | |
| 105N | 150P | 156T | 159V | 179N | 198L | | | | |
| 105N | 150P | 156T | 159V | 179N | 180T | | | | |
| 95K | 179N | 180T | 198L | 227E | 240S | | | | |
| 95K | 150P | 159V | 179N | 198L | 240S | | | | |
| 95K | 150P | 159V | 179N | 198L | 227E | | | | |
| 95K | 150P | 159V | 179N | 180T | 240S | | | | |
| 95K | 150P | 159V | 179N | 180T | 227E | | | | |
| 95K | 150P | 159V | 179N | 180T | 198L | | | | |
| 95K | 150P | 156K | 198L | 227E | 240S | | | | |
| 95K | 150P | 156K | 180T | 227E | 240S | | | | |
| 95K | 150P | 156K | 180T | 198L | 240S | | | | |
| 95K | 150P | 156K | 180T | 198L | 227E | | | | |
| 95K | 150P | 156K | 179N | 227E | 240S | | | | |
| 95K | 150P | 156K | 179N | 198L | 240S | | | | |
| 95K | 150P | 156K | 179N | 198L | 227E | | | | |
| 95K | 150P | 156K | 179N | 180T | 240S | | | | |
| 95K | 150P | 156K | 179N | 180T | 227E | | | | |
| 95K | 150P | 156K | 179N | 180T | 198L | | | | |
| 95K | 150P | 156K | 159V | 227E | 240S | | | | |
| 95K | 150P | 156K | 159V | 198L | 240S | | | | |
| 95K | 150P | 156K | 159V | 198L | 227E | | | | |
| 95K | 150P | 156K | 159V | 180T | 240S | | | | |
| 95K | 150P | 156K | 159V | 180T | 227E | | | | |
| 95K | 150P | 156K | 159V | 180T | 198L | | | | |
| 95K | 150P | 156K | 159V | 179N | 240S | | | | |
| 95K | 150P | 156K | 159V | 179N | 227E | | | | |
| 95K | 150P | 156K | 159V | 179N | 198L | | | | |
| 95K | 150P | 156K | 159V | 179N | 180T | | | | |
| 95K | 150P | 156T | 198L | 227E | 240S | | | | |
| 95K | 150P | 156T | 180T | 227E | 240S | | | | |
| 95K | 150P | 156T | 180T | 198L | 240S | | | | |
| 95K | 150P | 156T | 180T | 198L | 227E | | | | |
| 95K | 150P | 156T | 179N | 227E | 240S | | | | |
| 95K | 150P | 156T | 179N | 198L | 240S | | | | |
| 95K | 150P | 156T | 179N | 198L | 227E | | | | |
| 95K | 150P | 156T | 179N | 180T | 240S | | | | |
| 95K | 150P | 156T | 179N | 180T | 227E | | | | |
| 95K | 150P | 156T | 179N | 180T | 198L | | | | |
| 95K | 150P | 156T | 159V | 227E | 240S | | | | |
| 95K | 150P | 156T | 159V | 198L | 240S | | | | |
| 95K | 150P | 156T | 159V | 198L | 227E | | | | |
| 95K | 150P | 156T | 159V | 180T | 227E | | | | |
| 95K | 150P | 156T | 159V | 180T | 198L | | | | |
| 95K | 150P | 156T | 159V | 179N | 240S | | | | |
| 95K | 150P | 156T | 159V | 179N | 227E | | | | |
| 95K | 150P | 156T | 159V | 179N | 198L | | | | |
| 95K | 150P | 156T | 159V | 179N | 180T | | | | |
| 95K | 105N | 180T | 198L | 227E | 240S | | | | |
| 150P | 156T | 159V | 179N | 198L | 240S | | | | |
| 159V | 179N | 180T | 198L | 227E | 240S | | | | |
| 156K | 179N | 180T | 198L | 227E | 240S | | | | |
| 156K | 159V | 180T | 198L | 227E | 240S | | | | |
| 156K | 159V | 179N | 198L | 227E | 240S | | | | |
| 156K | 159V | 179N | 180T | 227E | 240S | | | | |
| 156K | 159V | 179N | 180T | 198L | 240S | | | | |
| 105N | 159V | 179N | 198L | 227E | 240S | | | | |
| 105N | 159V | 179N | 180T | 227E | 240S | | | | |
| 105N | 159V | 179N | 180T | 198L | 240S | | | | |
| 105N | 159V | 179N | 180T | 198L | 227E | | | | |
| 105N | 156K | 180T | 198L | 227E | 240S | | | | |
| 105N | 156K | 179N | 198L | 227E | 240S | | | | |
| 105N | 156K | 179N | 180T | 227E | 240S | | | | |
| 105N | 156K | 179N | 180T | 198L | 240S | | | | |
| 105N | 156K | 179N | 180T | 198L | 227E | | | | |
| 105N | 156K | 159V | 198L | 227E | 240S | | | | |
| 105N | 156K | 159V | 180T | 227E | 240S | | | | |
| 105N | 156K | 159V | 180T | 198L | 240S | | | | |
| 105N | 156K | 159V | 180T | 198L | 227E | | | | |
| 105N | 156K | 159V | 179N | 227E | 240S | | | | |
| 105N | 156K | 159V | 179N | 198L | 240S | | | | |
| 105N | 156K | 159V | 179N | 198L | 227E | | | | |
| 105N | 156K | 159V | 179N | 180T | 240S | | | | |
| 105N | 156K | 159V | 179N | 180T | 227E | | | | |
| 105N | 156K | 159V | 179N | 180T | 198L | | | | |
| 105N | 156T | 180T | 198L | 227E | 240S | | | | |
| 105N | 156T | 179N | 198L | 227E | 240S | | | | |
| 105N | 156T | 179N | 180T | 227E | 240S | | | | |
| 105N | 156T | 179N | 180T | 198L | 240S | | | | |
| 105N | 156T | 179N | 180T | 198L | 227E | | | | |
| 105N | 156T | 159V | 198L | 227E | 240S | | | | |
| 105N | 156T | 159V | 180T | 227E | 240S | | | | |
| 105N | 156T | 159V | 180T | 198L | 240S | | | | |
| 105N | 156T | 159V | 180T | 198L | 227E | | | | |
| 105N | 156T | 159V | 179N | 227E | 240S | | | | |
| 105N | 156T | 159V | 179N | 198L | 240S | | | | |
| 105N | 156T | 159V | 179N | 198L | 227E | | | | |
| 105N | 156T | 159V | 179N | 180T | 240S | | | | |
| 105N | 156T | 159V | 179N | 180T | 227E | | | | |
| 105N | 156T | 159V | 179N | 180T | 198L | | | | |
| 105N | 150P | 180T | 198L | 227E | 240S | | | | |
| 105N | 150P | 179N | 198L | 227E | 240S | | | | |
| 105N | 150P | 179N | 180T | 227E | 240S | | | | |
| 105N | 150P | 179N | 180T | 198L | 240S | | | | |
| 105N | 150P | 156T | 159V | 180T | 227E | | | | |
| 105N | 150P | 159V | 179N | 227E | 240S | | | | |
| 105N | 150P | 159V | 179N | 198L | 240S | | | | |
| 105N | 150P | 159V | 179N | 198L | 227E | | | | |
| 105N | 150P | 159V | 179N | 180T | 240S | | | | |
| 105N | 150P | 159V | 179N | 180T | 227E | | | | |
| 105N | 150P | 159V | 179N | 180T | 198L | | | | |
| 95K | 159V | 180T | 198L | 227E | 240S | | | | |
| 95K | 159V | 179N | 198L | 227E | 240S | | | | |
| 95K | 159V | 179N | 180T | 227E | 240S | | | | |
| 95K | 159V | 179N | 180T | 198L | 240S | | | | |
| 95K | 159V | 179N | 180T | 198L | 227E | | | | |
| 95K | 156K | 180T | 198L | 227E | 240S | | | | |
| 95K | 156K | 179N | 198L | 227E | 240S | | | | |
| 95K | 156K | 179N | 180T | 227E | 240S | | | | |
| 95K | 156K | 179N | 180T | 198L | 240S | | | | |
| 95K | 156K | 179N | 180T | 198L | 227E | | | | |
| 95K | 156K | 159V | 198L | 227E | 240S | | | | |
| 95K | 156K | 159V | 180T | 227E | 240S | | | | |
| 95K | 156K | 159V | 180T | 198L | 240S | | | | |
| 95K | 156K | 159V | 180T | 198L | 227E | | | | |
| 95K | 156K | 159V | 179N | 227E | 240S | | | | |
| 95K | 156K | 159V | 179N | 198L | 240S | | | | |
| 95K | 156K | 159V | 179N | 198L | 227E | | | | |
| 95K | 156K | 159V | 179N | 180T | 240S | | | | |
| 95K | 156K | 159V | 179N | 180T | 227E | | | | |
| 95K | 156K | 159V | 179N | 180T | 198L | | | | |
| 95K | 156T | 180T | 198L | 227E | 240S | | | | |
| 95K | 156T | 179N | 198L | 227E | 240S | | | | |
| 95K | 156T | 179N | 180T | 227E | 240S | | | | |
| 95K | 156T | 179N | 180T | 198L | 240S | | | | |
| 95K | 156T | 179N | 180T | 198L | 227E | | | | |
| 95K | 156T | 159V | 198L | 227E | 240S | | | | |
| 95K | 156T | 159V | 180T | 227E | 240S | | | | |
| 95K | 156T | 159V | 180T | 198L | 240S | | | | |
| 95K | 156T | 159V | 180T | 198L | 227E | | | | |
| 95K | 156T | 159V | 179N | 227E | 240S | | | | |
| 95K | 156T | 159V | 179N | 198L | 240S | | | | |
| 95K | 156T | 159V | 179N | 198L | 227E | | | | |
| 95K | 156T | 159V | 179N | 180T | 240S | | | | |
| 95K | 156T | 159V | 179N | 180T | 227E | | | | |
| 95K | 156T | 159V | 179N | 180T | 198L | | | | |
| 95K | 150P | 180T | 198L | 227E | 240S | | | | |
| 95K | 150P | 179N | 198L | 227E | 240S | | | | |
| 95K | 150P | 179N | 180T | 227E | 240S | | | | |
| 95K | 150P | 156T | 159V | 180T | 240S | | | | |
| 95K | 150P | 179N | 180T | 198L | 227E | | | | |
| 95K | 150P | 159V | 198L | 227E | 240S | | | | |
| 95K | 150P | 159V | 180T | 227E | 240S | | | | |
| 95K | 150P | 159V | 180T | 198L | 240S | | | | |
| 95K | 150P | 159V | 180T | 198L | 227E | | | | |
| 95K | 150P | 159V | 179N | 227E | 240S | | | | |
| 95K | 105N | 179N | 198L | 227E | 240S | | | | |
| 95K | 105N | 179N | 180T | 227E | 240S | | | | |
| 95K | 105N | 179N | 180T | 198L | 240S | | | | |
| 95K | 105N | 179N | 180T | 198L | 227E | | | | |
| 95K | 105N | 159V | 198L | 227E | 240S | | | | |
| 95K | 105N | 159V | 180T | 227E | 240S | | | | |
| 95K | 105N | 159V | 180T | 198L | 240S | | | | |
| 95K | 105N | 159V | 180T | 198L | 227E | | | | |
| 95K | 105N | 159V | 179N | 227E | 240S | | | | |
| 95K | 105N | 159V | 179N | 198L | 240S | | | | |
| 95K | 105N | 159V | 179N | 198L | 227E | | | | |
| 95K | 105N | 159V | 179N | 180T | 240S | | | | |
| 95K | 105N | 159V | 179N | 180T | 227E | | | | |
| 95K | 105N | 159V | 179N | 180T | 198L | | | | |
| 95K | 105N | 156K | 198L | 227E | 240S | | | | |
| 95K | 105N | 156K | 180T | 227E | 240S | | | | |
| 95K | 105N | 156K | 180T | 198L | 240S | | | | |
| 95K | 105N | 156K | 180T | 198L | 227E | | | | |
| 95K | 105N | 156K | 179N | 227E | 240S | | | | |
| 95K | 105N | 156K | 179N | 198L | 240S | | | | |
| 95K | 105N | 156K | 179N | 198L | 227E | | | | |
| 95K | 105N | 156K | 179N | 180T | 240S | | | | |
| 95K | 105N | 156K | 179N | 180T | 227E | | | | |
| 95K | 105N | 156K | 179N | 180T | 198L | | | | |
| 95K | 105N | 156K | 159V | 227E | 240S | | | | |
| 95K | 105N | 156K | 159V | 198L | 240S | | | | |
| 95K | 105N | 156K | 159V | 198L | 227E | | | | |
| 95K | 105N | 156K | 159V | 180T | 240S | | | | |
| 95K | 105N | 156K | 159V | 180T | 227E | | | | |
| 95K | 105N | 156K | 159V | 180T | 198L | | | | |
| 95K | 105N | 156K | 159V | 179N | 240S | | | | |
| 95K | 105N | 156K | 159V | 179N | 227E | | | | |
| 95K | 105N | 156K | 159V | 179N | 198L | | | | |
| 95K | 105N | 156K | 159V | 179N | 180T | | | | |
| 95K | 105N | 156T | 198L | 227E | 240S | | | | |
| 95K | 105N | 156T | 180T | 227E | 240S | | | | |
| 95K | 105N | 156T | 180T | 198L | 240S | | | | |
| 95K | 105N | 156T | 179N | 227E | 240S | | | | |
| 95K | 105N | 156T | 179N | 198L | 240S | | | | |
| 95K | 105N | 156T | 179N | 198L | 227E | | | | |
| 95K | 105N | 156T | 179N | 180T | 240S | | | | |
| 95K | 105N | 156T | 179N | 180T | 227E | | | | |
| 95K | 105N | 156T | 179N | 180T | 198L | | | | |
| 95K | 105N | 156T | 159V | 227E | 240S | | | | |
| 95K | 105N | 156T | 159V | 198L | 240S | | | | |
| 95K | 105N | 156T | 159V | 198L | 227E | | | | |
| 95K | 105N | 156T | 159V | 180T | 240S | | | | |
| 95K | 105N | 156T | 159V | 180T | 227E | | | | |
| 95K | 105N | 156T | 159V | 180T | 198L | | | | |
| 95K | 105N | 156T | 159V | 179N | 240S | | | | |
| 95K | 105N | 156T | 159V | 179N | 227E | | | | |
| 95K | 105N | 156T | 159V | 179N | 198L | | | | |
| 95K | 105N | 156T | 159V | 179N | 180T | | | | |
| 95K | 105N | 150P | 198L | 227E | 240S | | | | |
| 95K | 105N | 150P | 180T | 227E | 240S | | | | |
| 95K | 105N | 150P | 180T | 198L | 240S | | | | |
| 95K | 105N | 150P | 180T | 198L | 227E | | | | |
| 95K | 105N | 150P | 179N | 227E | 240S | | | | |
| 95K | 105N | 150P | 179N | 198L | 240S | | | | |
| 95K | 105N | 150P | 179N | 198L | 227E | | | | |
| 95K | 105N | 150P | 179N | 180T | 240S | | | | |
| 95K | 105N | 150P | 179N | 180T | 227E | | | | |
| 95K | 105N | 150P | 179N | 180T | 198L | | | | |
| 95K | 105N | 150P | 159V | 227E | 240S | | | | |
| 95K | 105N | 150P | 159V | 198L | 240S | | | | |
| 95K | 105N | 150P | 159V | 198L | 227E | | | | |
| 95K | 105N | 150P | 159V | 180T | 240S | | | | |
| 95K | 105N | 150P | 159V | 180T | 227E | | | | |
| 95K | 105N | 150P | 159V | 180T | 198L | | | | |
| 95K | 105N | 150P | 159V | 179N | 240S | | | | |
| 95K | 105N | 150P | 159V | 179N | 227E | | | | |
| 95K | 105N | 150P | 156K | 179N | 198L | | | | |
| 95K | 105N | 150P | 156K | 179N | 180T | | | | |
| 95K | 105N | 150P | 156K | 159V | 240S | | | | |
| 95K | 105N | 150P | 156K | 159V | 227E | | | | |
| 95K | 105N | 150P | 156K | 159V | 198L | | | | |
| 95K | 105N | 150P | 156K | 159V | 180T | | | | |
| 95K | 105N | 150P | 156K | 159V | 179N | | | | |
| 95K | 105N | 150P | 159V | 179N | 198L | | | | |
| 95K | 105N | 150P | 159V | 179N | 180T | | | | |
| 95K | 105N | 150P | 156K | 227E | 240S | | | | |
| 95K | 105N | 150P | 156K | 198L | 240S | | | | |
| 95K | 105N | 150P | 156K | 198L | 227E | | | | |
| 95K | 105N | 150P | 156K | 180T | 240S | | | | |
| 95K | 105N | 150P | 156K | 180T | 227E | | | | |
| 95K | 105N | 150P | 156K | 180T | 198L | | | | |
| 95K | 105N | 150P | 156T | 227E | 240S | | | | |
| 95K | 105N | 150P | 156T | 198L | 240S | | | | |
| 95K | 105N | 150P | 156T | 198L | 227E | | | | |
| 95K | 105N | 150P | 156T | 180T | 240S | | | | |
| 95K | 105N | 150P | 156T | 180T | 227E | | | | |
| 95K | 105N | 150P | 156T | 180T | 198L | | | | |
| 95K | 105N | 150P | 156T | 179N | 240S | | | | |
| 95K | 105N | 150P | 156T | 179N | 227E | | | | |
| 95K | 105N | 150P | 156T | 179N | 198L | | | | |
| 95K | 105N | 150P | 156T | 179N | 180T | | | | |
| 95K | 105N | 150P | 156T | 159V | 240S | | | | |
| 95K | 105N | 150P | 156T | 159V | 227E | | | | |
| 95K | 105N | 150P | 156T | 159V | 198L | | | | |
| 95K | 105N | 150P | 156T | 159V | 180T | | | | |
| 95K | 105N | 150P | 156T | 159V | 179N | | | | |
| 95K | 105N | 150P | 156K | 179N | 240S | | | | |
| 95K | 105N | 150P | 156K | 179N | 227E | | | | |
| 95K | 105N | 156T | 180T | 198L | 227E | | | | |
| 156K | 159V | 179N | 180T | 198L | 227E | 240S | | | |
| 156T | 159V | 179N | 180T | 198L | 227E | 240S | | | |
| 150P | 159V | 179N | 180T | 198L | 227E | 240S | | | |
| 150P | 156K | 179N | 180T | 198L | 227E | 240S | | | |
| 150P | 156K | 159V | 180T | 198L | 227E | 240S | | | |
| 150P | 156K | 159V | 179N | 198L | 227E | 240S | | | |
| 150P | 156K | 159V | 179N | 180T | 227E | 240S | | | |
| 150P | 156K | 159V | 179N | 180T | 198L | 240S | | | |
| 150P | 156K | 159V | 179N | 180T | 198L | 227E | | | |
| 150P | 156T | 179N | 180T | 198L | 227E | 240S | | | |
| 150P | 156T | 159V | 180T | 198L | 227E | 240S | | | |
| 150P | 156T | 159V | 179N | 198L | 227E | 240S | | | |
| 150P | 156T | 159V | 179N | 180T | 227E | 240S | | | |
| 150P | 156T | 159V | 179N | 180T | 198L | 240S | | | |
| 150P | 156T | 159V | 179N | 180T | 198L | 227E | | | |
| 105N | 156K | 179N | 180T | 198L | 227E | 240S | | | |
| 105N | 156K | 159V | 180T | 198L | 227E | 240S | | | |
| 105N | 156K | 159V | 179N | 198L | 227E | 240S | | | |
| 105N | 156K | 159V | 179N | 180T | 198L | 240S | | | |
| 105N | 156K | 159V | 179N | 180T | 198L | 227E | | | |
| 105N | 159V | 179N | 180T | 198L | 227E | 240S | | | |
| 105N | 156K | 159V | 179N | 180T | 227E | 240S | | | |
| 105N | 156T | 159V | 179N | 180T | 198L | 227E | | | |
| 105N | 156T | 179N | 180T | 198L | 227E | 240S | | | |
| 105N | 150P | 179N | 180T | 198L | 227E | 240S | | | |
| 105N | 150P | 159V | 180T | 198L | 227E | 240S | | | |
| 105N | 150P | 159V | 179N | 198L | 227E | 240S | | | |
| 105N | 150P | 159V | 179N | 180T | 227E | 240S | | | |
| 105N | 150P | 159V | 179N | 180T | 198L | 240S | | | |
| 105N | 150P | 159V | 179N | 180T | 198L | 227E | | | |
| 105N | 150P | 156K | 180T | 198L | 227E | 240S | | | |
| 105N | 150P | 156K | 179N | 198L | 227E | 240S | | | |
| 105N | 150P | 156K | 179N | 180T | 227E | 240S | | | |
| 105N | 150P | 156K | 179N | 180T | 198L | 240S | | | |
| 105N | 150P | 156K | 179N | 180T | 198L | 227E | | | |
| 105N | 150P | 156K | 159V | 198L | 227E | 240S | | | |
| 105N | 150P | 156K | 159V | 180T | 227E | 240S | | | |
| 105N | 150P | 156K | 159V | 180T | 198L | 240S | | | |
| 105N | 150P | 156K | 159V | 180T | 198L | 227E | | | |
| 105N | 150P | 156K | 159V | 179N | 227E | 240S | | | |
| 105N | 150P | 156K | 159V | 179N | 198L | 240S | | | |
| 105N | 150P | 156K | 159V | 179N | 198L | 227E | | | |
| 105N | 150P | 156K | 159V | 179N | 180T | 240S | | | |
| 105N | 150P | 156K | 159V | 179N | 180T | 227E | | | |
| 105N | 150P | 156K | 159V | 179N | 180T | 198L | | | |
| 105N | 150P | 156T | 179N | 198L | 227E | 240S | | | |
| 105N | 150P | 156T | 179N | 180T | 227E | 240S | | | |
| 105N | 150P | 156T | 179N | 180T | 198L | 240S | | | |
| 105N | 150P | 156T | 179N | 180T | 198L | 227E | | | |
| 105N | 150P | 156T | 159V | 198L | 227E | 240S | | | |
| 105N | 150P | 156T | 159V | 180T | 227E | 240S | | | |
| 105N | 150P | 156T | 159V | 180T | 198L | 240S | | | |
| 105N | 150P | 156T | 159V | 180T | 198L | 227E | | | |
| 105N | 150P | 156T | 159V | 179N | 198L | 240S | | | |
| 105N | 150P | 156T | 159V | 179N | 198L | 227E | | | |
| 105N | 150P | 156T | 180T | 198L | 227E | 240S | | | |
| 105N | 156T | 159V | 179N | 198L | 227E | 240S | | | |
| 105N | 156T | 159V | 179N | 180T | 198L | 240S | | | |
| 105N | 156T | 159V | 179N | 180T | 227E | 240S | | | |
| 105N | 156T | 159V | 180T | 198L | 227E | 240S | | | |
| 95K | 156T | 159V | 179N | 198L | 227E | 240S | | | |
| 95K | 156T | 159V | 179N | 180T | 227E | 240S | | | |
| 95K | 156T | 159V | 179N | 180T | 198L | 240S | | | |
| 95K | 156T | 159V | 179N | 180T | 198L | 227E | | | |
| 95K | 150P | 179N | 180T | 198L | 227E | 240S | | | |
| 95K | 150P | 159V | 180T | 198L | 227E | 240S | | | |
| 95K | 150P | 159V | 179N | 198L | 227E | 240S | | | |
| 95K | 150P | 159V | 179N | 180T | 227E | 240S | | | |
| 95K | 150P | 159V | 179N | 180T | 198L | 240S | | | |
| 95K | 150P | 159V | 179N | 180T | 198L | 227E | | | |
| 105N | 150P | 156T | 159V | 179N | 227E | 240S | | | |
| 105N | 150P | 156T | 159V | 179N | 180T | 240S | | | |
| 105N | 150P | 156T | 159V | 179N | 180T | 227E | | | |
| 105N | 150P | 156T | 159V | 179N | 180T | 198L | | | |
| 95K | 159V | 179N | 180T | 198L | 227E | 240S | | | |
| 95K | 156K | 179N | 180T | 198L | 227E | 240S | | | |
| 95K | 156K | 159V | 180T | 198L | 227E | 240S | | | |
| 95K | 156K | 159V | 179N | 198L | 227E | 240S | | | |
| 95K | 156K | 159V | 179N | 180T | 227E | 240S | | | |
| 95K | 156K | 159V | 179N | 180T | 198L | 240S | | | |
| 95K | 156K | 159V | 179N | 180T | 198L | 227E | | | |
| 95K | 156T | 179N | 180T | 198L | 227E | 240S | | | |
| 95K | 156T | 159V | 180T | 198L | 227E | 240S | | | |
| 95K | 150P | 156K | 159V | 179N | 180T | 227E | | | |
| 95K | 150P | 156K | 159V | 179N | 180T | 198L | | | |
| 95K | 150P | 156T | 180T | 198L | 227E | 240S | | | |
| 95K | 150P | 156T | 179N | 198L | 227E | 240S | | | |
| 95K | 150P | 156T | 179N | 180T | 227E | 240S | | | |
| 95K | 150P | 156T | 179N | 180T | 198L | 240S | | | |
| 95K | 150P | 156T | 179N | 180T | 198L | 227E | | | |
| 95K | 150P | 156T | 159V | 198L | 227E | 240S | | | |
| 95K | 150P | 156T | 159V | 180T | 227E | 240S | | | |
| 95K | 150P | 156T | 159V | 180T | 198L | 240S | | | |
| 95K | 150P | 156T | 159V | 180T | 198L | 227E | | | |
| 95K | 150P | 156T | 159V | 179N | 227E | 240S | | | |
| 95K | 150P | 156T | 159V | 179N | 198L | 240S | | | |
| 95K | 150P | 156T | 159V | 179N | 198L | 227E | | | |
| 95K | 150P | 156T | 159V | 179N | 180T | 240S | | | |
| 95K | 150P | 156T | 159V | 179N | 180T | 227E | | | |
| 95K | 150P | 156K | 180T | 198L | 227E | 240S | | | |
| 95K | 150P | 156K | 179N | 198L | 227E | 240S | | | |
| 95K | 150P | 156K | 179N | 180T | 227E | 240S | | | |
| 95K | 150P | 156K | 179N | 180T | 198L | 240S | | | |
| 95K | 150P | 156K | 179N | 180T | 198L | 227E | | | |
| 95K | 150P | 156K | 159V | 198L | 227E | 240S | | | |
| 95K | 150P | 156K | 159V | 180T | 227E | 240S | | | |
| 95K | 150P | 156K | 159V | 180T | 198L | 240S | | | |
| 95K | 150P | 156K | 159V | 180T | 198L | 227E | | | |
| 95K | 150P | 156K | 159V | 179N | 227E | 240S | | | |
| 95K | 150P | 156K | 159V | 179N | 198L | 240S | | | |
| 95K | 150P | 156K | 159V | 179N | 198L | 227E | | | |
| 95K | 150P | 156K | 159V | 179N | 180T | 240S | | | |
| 95K | 105N | 156K | 180T | 198L | 227E | 240S | | | |
| 95K | 105N | 156K | 179N | 198L | 227E | 240S | | | |
| 95K | 105N | 156K | 179N | 180T | 227E | 240S | | | |
| 95K | 105N | 156K | 179N | 180T | 198L | 240S | | | |
| 95K | 105N | 156K | 179N | 180T | 198L | 227E | | | |
| 95K | 105N | 156K | 159V | 198L | 227E | 240S | | | |
| 95K | 105N | 156K | 159V | 180T | 227E | 240S | | | |
| 95K | 105N | 156K | 159V | 180T | 198L | 240S | | | |
| 95K | 105N | 156K | 159V | 180T | 198L | 227E | | | |
| 95K | 105N | 156K | 159V | 179N | 227E | 240S | | | |
| 95K | 105N | 156K | 159V | 179N | 198L | 240S | | | |
| 95K | 105N | 156K | 159V | 179N | 198L | 227E | | | |
| 95K | 105N | 156K | 159V | 179N | 180T | 240S | | | |
| 95K | 105N | 156K | 159V | 179N | 180T | 227E | | | |
| 95K | 105N | 156K | 159V | 179N | 180T | 198L | | | |
| 95K | 105N | 156T | 180T | 198L | 227E | 240S | | | |
| 95K | 150P | 156T | 159V | 179N | 180T | 198L | | | |
| 95K | 105N | 179N | 180T | 198L | 227E | 240S | | | |
| 95K | 105N | 159V | 180T | 198L | 227E | 240S | | | |
| 95K | 105N | 159V | 179N | 198L | 227E | 240S | | | |
| 95K | 105N | 159V | 179N | 180T | 227E | 240S | | | |
| 95K | 105N | 159V | 179N | 180T | 198L | 240S | | | |
| 95K | 105N | 159V | 179N | 180T | 198L | 227E | | | |
| 95K | 105N | 156T | 159V | 180T | 198L | 227E | | | |
| 95K | 105N | 156T | 159V | 179N | 227E | 240S | | | |
| 95K | 105N | 156T | 159V | 179N | 198L | 240S | | | |
| 95K | 105N | 156T | 159V | 179N | 198L | 227E | | | |
| 95K | 105N | 156T | 159V | 179N | 180T | 240S | | | |
| 95K | 105N | 156T | 159V | 179N | 180T | 227E | | | |
| 95K | 105N | 156T | 159V | 179N | 180T | 198L | | | |
| 95K | 105N | 150P | 180T | 198L | 227E | 240S | | | |
| 95K | 105N | 150P | 179N | 198L | 227E | 240S | | | |
| 95K | 105N | 150P | 179N | 180T | 227E | 240S | | | |
| 95K | 105N | 150P | 179N | 180T | 198L | 240S | | | |
| 95K | 105N | 150P | 179N | 180T | 198L | 227E | | | |
| 95K | 105N | 150P | 159V | 198L | 227E | 240S | | | |
| 95K | 105N | 150P | 159V | 180T | 227E | 240S | | | |
| 95K | 105N | 150P | 159V | 180T | 198L | 240S | | | |
| 95K | 105N | 150P | 159V | 180T | 198L | 227E | | | |
| 95K | 105N | 150P | 159V | 179N | 227E | 240S | | | |
| 95K | 105N | 150P | 159V | 179N | 198L | 240S | | | |
| 95K | 105N | 150P | 159V | 179N | 198L | 227E | | | |
| 95K | 105N | 150P | 159V | 179N | 180T | 240S | | | |
| 95K | 105N | 150P | 159V | 179N | 180T | 227E | | | |
| 95K | 105N | 150P | 159V | 179N | 180T | 198L | | | |
| 95K | 105N | 150P | 156K | 198L | 227E | 240S | | | |
| 95K | 105N | 150P | 156T | 179N | 227E | 240S | | | |
| 95K | 105N | 150P | 156T | 179N | 198L | 240S | | | |
| 95K | 105N | 150P | 156T | 179N | 198L | 227E | | | |
| 95K | 105N | 150P | 156T | 179N | 180T | 240S | | | |
| 95K | 105N | 150P | 156T | 179N | 180T | 227E | | | |
| 95K | 105N | 150P | 156T | 179N | 180T | 198L | | | |
| 95K | 105N | 150P | 156T | 159V | 227E | 240S | | | |
| 95K | 105N | 150P | 156T | 159V | 198L | 240S | | | |
| 95K | 105N | 150P | 156T | 159V | 198L | 227E | | | |
| 95K | 105N | 150P | 156T | 159V | 180T | 240S | | | |
| 95K | 105N | 150P | 156T | 159V | 180T | 227E | | | |
| 95K | 105N | 150P | 156T | 159V | 180T | 198L | | | |
| 95K | 105N | 150P | 156T | 159V | 179N | 240S | | | |
| 95K | 105N | 150P | 156T | 159V | 179N | 227E | | | |
| 95K | 105N | 150P | 156T | 159V | 179N | 198L | | | |
| 95K | 105N | 150P | 156T | 159V | 179N | 180T | | | |
| 95K | 105N | 156T | 179N | 198L | 227E | 240S | | | |
| 95K | 105N | 156T | 179N | 180T | 227E | 240S | | | |
| 95K | 105N | 156T | 179N | 180T | 198L | 240S | | | |
| 95K | 105N | 156T | 179N | 180T | 198L | 227E | | | |
| 95K | 105N | 156T | 159V | 198L | 227E | 240S | | | |
| 95K | 105N | 156T | 159V | 180T | 227E | 240S | | | |
| 95K | 105N | 156T | 159V | 180T | 198L | 240S | | | |
| 95K | 105N | 150P | 156K | 180T | 227E | 240S | | | |
| 95K | 105N | 150P | 156K | 180T | 198L | 240S | | | |
| 95K | 105N | 150P | 156K | 180T | 198L | 227E | | | |
| 95K | 105N | 150P | 156K | 179N | 227E | 240S | | | |
| 95K | 105N | 150P | 156K | 179N | 198L | 240S | | | |
| 95K | 105N | 150P | 156K | 179N | 198L | 227E | | | |
| 95K | 105N | 150P | 156K | 179N | 180T | 240S | | | |
| 95K | 105N | 150P | 156K | 179N | 180T | 227E | | | |
| 95K | 105N | 150P | 156K | 179N | 180T | 198L | | | |
| 95K | 105N | 150P | 156K | 159V | 227E | 240S | | | |
| 95K | 105N | 150P | 156K | 159V | 198L | 240S | | | |
| 95K | 105N | 150P | 156K | 159V | 198L | 227E | | | |
| 95K | 105N | 150P | 156K | 159V | 180T | 240S | | | |
| 95K | 105N | 150P | 156K | 159V | 180T | 227E | | | |
| 95K | 105N | 150P | 156K | 159V | 180T | 198L | | | |
| 95K | 105N | 150P | 156K | 159V | 179N | 240S | | | |
| 95K | 105N | 150P | 156K | 159V | 179N | 227E | | | |
| 95K | 105N | 150P | 156K | 159V | 179N | 198L | | | |
| 95K | 105N | 150P | 156K | 159V | 179N | 180T | | | |
| 95K | 105N | 150P | 156T | 198L | 227E | 240S | | | |
| 95K | 105N | 150P | 156T | 180T | 227E | 240S | | | |
| 95K | 105N | 150P | 156T | 180T | 198L | 240S | | | |
| 95K | 105N | 150P | 156T | 180T | 198L | 227E | | | |
| 105N | 150P | 156K | 159V | 179N | 198L | 227E | 240S | | |
| 105N | 150P | 156K | 159V | 179N | 180T | 227E | 240S | | |
| 105N | 150P | 156K | 159V | 179N | 180T | 198L | 240S | | |
| 105N | 150P | 156K | 159V | 179N | 180T | 198L | 227E | | |
| 105N | 150P | 156T | 179N | 180T | 198L | 227E | 240S | | |
| 105N | 150P | 156T | 159V | 180T | 198L | 227E | 240S | | |
| 105N | 150P | 156T | 159V | 179N | 198L | 227E | 240S | | |
| 105N | 150P | 156T | 159V | 179N | 180T | 227E | 240S | | |
| 105N | 150P | 156T | 159V | 179N | 180T | 198L | 240S | | |
| 105N | 150P | 156T | 159V | 179N | 180T | 198L | 227E | | |
| 95K | 150P | 156T | 159V | 179N | 180T | 198L | 240S | | |
| 95K | 150P | 156T | 159V | 179N | 180T | 198L | 227E | | |
| 95K | 105N | 159V | 179N | 180T | 198L | 227E | 240S | | |
| 95K | 105N | 156K | 179N | 180T | 198L | 227E | 240S | | |
| 95K | 105N | 156K | 159V | 180T | 198L | 227E | 240S | | |
| 95K | 105N | 156K | 159V | 179N | 198L | 227E | 240S | | |
| 95K | 105N | 156K | 159V | 179N | 180T | 227E | 240S | | |
| 95K | 105N | 156K | 159V | 179N | 180T | 198L | 240S | | |
| 95K | 105N | 156K | 159V | 179N | 180T | 198L | 227E | | |
| 95K | 105N | 156T | 179N | 180T | 198L | 227E | 240S | | |
| 95K | 156K | 159V | 179N | 180T | 198L | 227E | 240S | | |
| 95K | 156T | 159V | 179N | 180T | 198L | 227E | 240S | | |
| 95K | 150P | 159V | 179N | 180T | 198L | 227E | 240S | | |
| 95K | 150P | 156K | 179N | 180T | 198L | 227E | 240S | | |
| 95K | 150P | 156K | 159V | 180T | 198L | 227E | 240S | | |
| 95K | 150P | 156K | 159V | 179N | 198L | 227E | 240S | | |
| 95K | 105N | 150P | 159V | 180T | 198L | 227E | 240S | | |
| 95K | 105N | 150P | 156T | 179N | 180T | 198L | 227E | | |
| 95K | 105N | 150P | 156T | 159V | 198L | 227E | 240S | | |
| 95K | 105N | 150P | 156T | 159V | 180T | 227E | 240S | | |
| 95K | 105N | 150P | 156T | 159V | 180T | 198L | 240S | | |
| 95K | 105N | 150P | 156T | 159V | 180T | 198L | 227E | | |
| 95K | 105N | 150P | 156T | 159V | 179N | 180T | 227E | | |
| 105N | 150P | 156K | 159V | 180T | 198L | 227E | 240S | | |
| 105N | 150P | 156K | 179N | 180T | 198L | 227E | 240S | | |
| 105N | 150P | 159V | 179N | 180T | 198L | 227E | 240S | | |
| 105N | 156K | 159V | 179N | 180T | 198L | 227E | 240S | | |
| 105N | 156T | 159V | 179N | 180T | 198L | 227E | 240S | | |
| 150P | 156K | 159V | 179N | 180T | 198L | 227E | 240S | | |
| 150P | 156T | 159V | 179N | 180T | 198L | 227E | 240S | | |
| 95K | 105N | 150P | 156T | 179N | 180T | 198L | 240S | | |
| 95K | 105N | 150P | 179N | 180T | 198L | 227E | 240S | | |
| 95K | 105N | 156T | 159V | 179N | 180T | 198L | 240S | | |
| 95K | 105N | 156T | 159V | 179N | 180T | 198L | 227E | | |
| 95K | 105N | 156T | 159V | 179N | 180T | 227E | 240S | | |
| 95K | 105N | 156T | 159V | 179N | 198L | 227E | 240S | | |
| 95K | 105N | 156T | 159V | 180T | 198L | 227E | 240S | | |
| 95K | 150P | 156K | 159V | 179N | 180T | 198L | 240S | | |
| 95K | 150P | 156K | 159V | 179N | 180T | 198L | 227E | | |
| 95K | 150P | 156K | 159V | 179N | 180T | 227E | 240S | | |
| 95K | 150P | 156T | 159V | 179N | 180T | 227E | 240S | | |
| 95K | 150P | 156T | 159V | 179N | 198L | 227E | 240S | | |
| 95K | 150P | 156T | 159V | 180T | 198L | 227E | 240S | | |
| 95K | 150P | 156T | 179N | 180T | 198L | 227E | 240S | | |
| 95K | 105N | 150P | 156T | 159V | 179N | 180T | 198L | | |
| 95K | 105N | 150P | 159V | 179N | 198L | 227E | 240S | | |
| 95K | 105N | 150P | 159V | 179N | 180T | 227E | 240S | | |
| 95K | 105N | 150P | 159V | 179N | 180T | 198L | 240S | | |
| 95K | 105N | 150P | 159V | 179N | 180T | 198L | 227E | | |
| 95K | 105N | 150P | 156K | 180T | 198L | 227E | 240S | | |
| 95K | 105N | 150P | 156K | 179N | 198L | 227E | 240S | | |
| 95K | 105N | 150P | 156K | 179N | 180T | 227E | 240S | | |
| 95K | 105N | 150P | 156K | 179N | 180T | 198L | 240S | | |
| 95K | 105N | 150P | 156K | 179N | 180T | 198L | 227E | | |
| 95K | 105N | 150P | 156K | 159V | 198L | 227E | 240S | | |
| 95K | 105N | 150P | 156K | 159V | 180T | 227E | 240S | | |
| 95K | 105N | 150P | 156K | 159V | 180T | 198L | 240S | | |
| 95K | 105N | 150P | 156K | 159V | 180T | 198L | 227E | | |
| 95K | 105N | 150P | 156K | 159V | 179N | 227E | 240S | | |
| 95K | 105N | 150P | 156K | 159V | 179N | 198L | 240S | | |
| 95K | 105N | 150P | 156K | 159V | 179N | 198L | 227E | | |
| 95K | 105N | 150P | 156K | 159V | 179N | 180T | 240S | | |
| 95K | 105N | 150P | 156K | 159V | 179N | 180T | 227E | | |
| 95K | 105N | 150P | 156K | 159V | 179N | 180T | 198L | | |
| 95K | 10SN | 150P | 156T | 180T | 198L | 227E | 240S | | |
| 95K | 105N | 150P | 156T | 179N | 198L | 227E | 240S | | |
| 95K | 105N | 150P | 156T | 179N | 180T | 227E | 240S | | |
| 95K | 105N | 150P | 156T | 159V | 179N | 227E | 240S | | |
| 95K | 105N | 150P | 156T | 159V | 179N | 198L | 240S | | |
| 95K | 105N | 150P | 156T | 159V | 179N | 198L | 227E | | |
| 95K | 105N | 150P | 156T | 159V | 179N | 180T | 240S | | |
| 105N | 150P | 156K | 159V | 179N | 180T | 198L | 227E | 240S | |
| 105N | 150P | 156T | 159V | 179N | 180T | 198L | 227E | 240S | |
| 95K | 150P | 156K | 159V | 179N | 180T | 198L | 227E | 240S | |
| 95K | 150P | 156T | 159V | 179N | 180T | 198L | 227E | 240S | |
| 95K | 105N | 156K | 159V | 179N | 180T | 198L | 227E | 240S | |
| 95K | 105N | 156T | 159V | 179N | 180T | 198L | 227E | 240S | |
| 95K | 105N | 150P | 159V | 179N | 180T | 198L | 227E | 240S | |
| 95K | 105N | 150P | 156K | 179N | 180T | 198L | 227E | 240S | |
| 95K | 105N | 150P | 156K | 159V | 180T | 198L | 227E | 240S | |
| 95K | 105N | 150P | 156K | 159V | 179N | 198L | 227E | 240S | |
| 95K | 105N | 150P | 156K | 159V | 179N | 180T | 227E | 240S | |
| 95K | 105N | 150P | 156K | 159V | 179N | 180T | 198L | 240S | |
| 95K | 105N | 150P | 156K | 159V | 179N | 180T | 198L | 227E | |
| 95K | 105N | 150P | 156T | 179N | 180T | 198L | 227E | 240S | |
| 95K | 105N | 150P | 156T | 159V | 180T | 198L | 227E | 240S | |
| 95K | 105N | 150P | 156T | 159V | 179N | 198L | 227E | 240S | |
| 95K | 105N | 150P | 156T | 159V | 179N | 180T | 227E | 240S | |
| 95K | 105N | 150P | 156T | 159V | 179N | 180T | 198L | 240S | |
| 95K | 105N | 150P | 156T | 159V | 179N | 180T | 198L | 227E | |
| 95K | 105N | 150P | 156K | 159V | 179N | 180T | 198L | 227E | 240S |
| 95K | 105N | 150P | 156T | 159V | 179N | 181T | 198L | 227E | 240S |

2. Screening

E. coli BL21 cells were transforred with the 1238 hMMP-1 CPS mutants in 96-well format and induced with IPTG at 25° C. as described in Example 1. The proteolytic activity of the hMMP-1 mutants were measured at 25° C. and 37° C. using fluorogenic peptide IX (R&D Systems, Minneapolis, Minn., Cat# ES010) as described in Example 2. Mutants that were active at 25° C. but showed minimal activity at 37° C. were identified as putative hits. The identified hits are set forth in Table 15B. The hits are sorted from the mutant with the lowest activity at 37° C. to the mutant with the highest activity at 37° C., i.e. wildtype. The results showed that many of the mutants, including combination mutants, exhibited 10% or less the activity of wildtype hMMP-1 at 25° C. The low activity was not due to problems with expression, since many of the mutants with no or low activities at 25° C. were expressed well. Several mutants (single and combination) exhibited substantial activity at 25° C. and also showed the best temperature profile (25° C./37° C.).

TABLE 15B
Temperature Profiles of Combination Mutants

| | | | | | 25° C.: | 37° C.: |
|---|---|---|---|---|---|---|
| | | | | Ratio | % Act. | % Act. |
| | | Avg. RFU | Avg. RFU | (25° C./ | of wt | of wt |
| Variant | SEQ ID NO | 25° C. | 37° C. | 37° C.) | 25° C. | 25° C. |
| D156K/G159V/D179N | 3507 | 1261.31 | 786.28 | 1.60 | 4.73 | 2.95 |
| R150P/V227E | 3508 | 1801.03 | 859.01 | 2.10 | 6.44 | 3.07 |
| D156T/V227E | 3509 | 2021.29 | 864.71 | 2.34 | 7.22 | 3.09 |
| G159V/A198L | 3510 | 1684.53 | 863.78 | 1.95 | 6.06 | 3.11 |
| D105N/A198L | 3511 | 1422.45 | 919.80 | 1.55 | 5.34 | 3.45 |
| L95K | 6 | 1389.81 | 969.67 | 1.43 | 5.00 | 3.49 |
| D179N/V227E | 3512 | 1446.86 | 948.41 | 1.53 | 5.43 | 3.56 |
| A198L/V227E | 3513 | 2740.04 | 1036.69 | 2.64 | 9.79 | 3.70 |
| EI80T/V227E | 3514 | 2549.76 | 1038.44 | 2.46 | 9.11 | 3.71 |
| D179N/A198L | 3515 | 1411.89 | 968.14 | 1.46 | 5.45 | 3.74 |
| D156K/D179N | 3516 | 1227.63 | 973.51 | 1.26 | 4.74 | 3.76 |
| D105N/R150P/ | 3517 | 1668.82 | 1002.65 | 1.66 | 6.26 | 3.76 |
| DIS6K/G159V/ | | | | | | |
| D179N/E180T | | | | | | |
| D105N/R150P/E180T | 3518 | 1846.75 | 1003.36 | 1.84 | 6.93 | 3.76 |
| G159V/I240S | 3519 | 2565.48 | 1031.27 | 2.49 | 9.45 | 3.80 |
| D156T/D1797N/I240S | 3520 | 1326.33 | 774.68 | 1.71 | 6.53 | 3.81 |
| D156T/G159V | 3521 | 1521.88 | 1048.30 | 1.45 | 5.71 | 3.93 |
| R150P/E180T | 3522 | 1636.14 | 1112.37 | 1.47 | 5.85 | 3.98 |
| D156T/D179N | 3523 | 3855.30 | 1049.65 | 3.67 | 14.72 | 4.01 |
| D179N/I240S | 3524 | 1890.16 | 826.28 | 2.29 | 9.30 | 4.07 |
| L95K/D156T/D179N | 3525 | 2075.52 | 1194.20 | 1.74 | 7.79 | 4.48 |
| D156T | 105 | 5564.55 | 1304.31 | 4.27 | 26.15 | 6.13 |
| G159V | 132 | 6330.31 | 1716.35 | 3.49 | 24.17 | 6.94 |
| G159V/D179N | 3526 | 4741.70 | 1896.45 | 2.50 | 17.79 | 7.12 |
| A198L | 305 | 4888.05 | 1555.23 | 3.14 | 22.97 | 7.31 |
| L95K/D105N/E180T | 3527 | 3640.58 | 2177.79 | 1.67 | 13.66 | 8.17 |
| R150P/D156T/A198L | 3528 | 2554.33 | 1770.29 | 1.44 | 12.00 | 8.32 |
| V227E | 384 | 21170.85 | 2439.36 | 9.01 | 76.14 | 8.45 |
| I240S | 488 | 5525.59 | 1486.79 | 3.72 | 33.21 | 8.94 |
| L95K/D105N/R150P/ | 3529 | 2930.99 | 2217.79 | 1.32 | 14.58 | 11.03 |
| D156T/G159V/A198L/ | | | | | | |
| V227E/I240S | | | | | | |
| L95K/R150P | 3530 | 6360.67 | 3108.26 | 2.05 | 30.68 | 14.99 |
| D105N/E180T | 3531 | 13018.08 | 4994.85 | 2.61 | 46.52 | 17.85 |
| R150P | 59 | 11979.01 | 4261.20 | 2.81 | 56.29 | 20.02 |
| D105N | 27 | 12356.79 | 4628.13 | 2.67 | 58.06 | 21.75 |
| E180T | 181 | 26456.92 | 11205.01 | 2.36 | 94.55 | 40.04 |
| Wildtype | 2 | 26316.84 | 22348.45 | 1.18 | 94.64 | 80.37 |

The results showing low activity at 25° C. for many of the combination mutants suggested that the combination mutants were altering the protein, such that their optimal temperature for activity was shifted below 25° C. To test this, the proteolytic activity of some of the combination mutants against the fluorogenic peptide IX was tested at 20° C., 25° C. and 37° C. Included among the combination mutants that were tested were: G159V/A198L; D156T/D179N; G159V/D179N; D179N/V227E; A198L/V227E; D156K/D179N; 179/240; and D156T/D179N/I240S. The results showed that several of the combination mutants had slightly higher activity at 20° C. than at 25° C., and little activity at 37° C. All of the mutants tested exhibited less activity (only about 33% of the activity or less) than wildtype MMP-1 at the corresponding temperature. One of the mutants, D156T/D179N, was tested and exhibited higher activity at 18° C. than wildtype.

### Example 4

### Reversibility of Enzymatic Activity Following Decrease in Temperature

In this example, the temperature sensitive hMMP-1 mutants that were confirmed in Example 2B were further assayed to determine whether enzymatic activity at 25° C. was reversible or irreversible following subsequent exposure to elevated temperatures followed by a return to 25° C. The hMMP-1 mutants were expressed in 14 ml culture tubes, as described in Example 2B. The putative hits were tested for their activities under five conditions: at 25° C., 34° C. or 37° C., and at 34° C. or 37° C. and subsequent re-exposure to the requsite temperature of 25° C. (see Table 16 for reaction conditions). Mutants that were active at 25° C., showed decreased activity when raised to 34° C. or 37° C. (i.e. the ratio of the activities at 25° C./34° C. or 25° C./37° C. is equal to or greater than 1.5), and exhibited a baseline activity when lowered again to 25° C. were scored as “Reversible Hits.” Mutants that were active at 25° C., showed decreased activity when raised to 34° C. or 37° C. (i.e. the ratio of the activities at 25° C./34° C. or 25° C./37° C. is equal to or greater than 1.5), and exhibited the same amount of decreased activity when lowered again to 25° C. were scored as “Irreversible Hits.”

A. Reaction Conditions

The reversibility of enzymatic activity of each hMMP-1 mutant was determined using the previously described fluorescence assay as modified below. In short, the 4 μl of the supernatant of each hMMP-1 mutant was diluted in TCNB with 1 mM APMA and transferred to a 96-well plate. Five different wells were prepared for each hMMP-1 mutant as set forth in Table 16. The solution was incubated at the initial reaction temperature (25° C., 34° C., or 37° C.) for 2 hours. This activation step cleaves the pro-peptide and generates mature hMMP-1.

Following activation, 100 μl of TCNB with 10 μM Mca-K-P-L-G-L-Dpa-A-R—NH₂ fluorescent substrate was added to each well and reaction conditions were as summarized in Table 16, below. Briefly, each hMMP-1 mutant was exposed to each of the five reaction conditions by incubation of the hMMP-1 mutant in the presence of the fluorogenic substrate for an hour at the initial temperature. For each mutant, baseline activity at 25° C., 34° C., or 37° C. was assessed by incubation with the substrate for an additional 1 hour (2 hour condition) or overnight (overnight condition), followed by fluorescence measurement. To assess the reversibility/irreversibility of activity, samples incubated for an initial 1 hour at 34° C., or 37° C. were lowered to 25° C. and allowed to incubate for either an hour (2 hour condition) or 16 hours (overnight condition), followed by fluorescence measurement. Wildtype hMMP-1 was used as a positive control and supernatant from cells transformed with only vector was used as a negative control. Fluorescence was detected by measuring fluorescence in a fluorescent plate reader at 320 nm exitation/405 nm emission. Relative fluorescence units (RFU) were determined. Duplicate reactions were performed for each sample, reaction temperature, and positive and negative control.

TABLE 16
Reaction Conditions

| | Initial | Incubation at | | |
|---|---|---|---|---|
| Condition | Temperature | 25° C. | 2 Hours | Overnight |
| 25° C. | 25° C. | — | 2 hours | overnight |
| 34° C. | 34° C. | — | 2 hours | overnight |
| 34° C. to | 34° C. | 25° C. | a) 34° C. for 1 | a) 34° C. for 1 |
| 25° C. | | | hour | hour |
| | | | b) 25° C. for 1 | b) 25° C. for |
| | | | hour | 16 hours |
| 37° C. | 37° C. | — | 2 hours | overnight |
| 37° C. to | 37° C. | 25° C. | a) 37° C. for 1 | a) 37° C. for 1 |
| 25° C. | | | hour | hour |
| | | | b) 25° C. for 1 | b) 25° C. for |
| | | | hour | 16 hours |

B. Results: Partially Reversible hMMP-1 Mutants

Twenty six hMMP-T mutants were determined to be partially reversible. Although the activity (in RFU) did not return to baseline activity observed at 25° C., an overall increase in activity was observed when the temperature was returned to 25° C. compared to activity at 34° C. or 37° C. The results are shown in Tables 17-20 below, which list the activities (in RFUs) and the ratios of the activities. Tables 17 and 18 summarize the results of reversibility at 34° C. or 37° C., respectively, of the hMMP-1 partially reversible mutants under the 2 hour condition. Tables 19 and 20 TO summarize the results of reversibility at 34° C. or 37° C., respectively, of the partially reversible hMMP-1 mutants under the overnight condition. The results are similar under all reaction conditions, temperature and time. The activity at 34° C. or 37° C. overnight is lower than the activity when incubated at 34° C. or 37° C. for one hour then 25° C. overnight. For example, the activity of E180Y at 34° C. is 6080 RFU but its activity at 34° C. then overnight at 25° C. increased to 8570 RFU (see Table 19, below).

TABLE 17
Partially Reversible hMMP-1 mutants (2 Hours, 34° C.)

| | | | | | | Ratio |
|---|---|---|---|---|---|---|
| | | | | RFU | Ratio | 25° C./ |
| hMMP-1 | SEQ ID | RFU | RFU | 34 to | 25° C./ | 34 to |
| mutation | NO | 25° C. | 34° C. | 25° C. | 34° C. | 25° C. |
| D105A | 39 | 5669.31 | 824.07 | 922.97 | 6.88 | 6.14 |
| D105F | 33 | 2980.00 | 623.89 | 725.03 | 4.78 | 4.11 |
| D105G | 32 | 8821.81 | 2759.24 | 2966.37 | 3.20 | 2.97 |
| D105S | 31 | 9355.63 | 4607.18 | 6681.63 | 2.03 | 1.40 |
| D105T | 29 | 4457.16 | 974.63 | 1534.71 | 4.57 | 2.90 |
| R150P | 59 | 8750.30 | 2315.11 | 2506.15 | 3.78 | 3.49 |
| G159T | 125 | 6704.95 | 2294.40 | 2344.57 | 2.92 | 2.86 |
| E180Y | 182 | 8557.09 | 4979.24 | 6224.87 | 1.72 | 1.37 |
| E180T | 181 | 7870.99 | 1532.35 | 1852.46 | 5.14 | 4.25 |
| E180F | 185 | 8508.13 | 3597.75 | 3915.71 | 2.36 | 2.17 |
| T185H | 233 | 5593.77 | 2278.26 | 2429.05 | 2.46 | 2.30 |
| T185Q | 238 | 7006.87 | 2250.58 | 2397.60 | 3.11 | 2.92 |
| T185A | 248 | 2474.96 | 663.82 | 822.83 | 3.73 | 3.01 |
| T185E | 232 | 3948.43 | 2088.15 | 1862.83 | 1.89 | 2.12 |
| N187R | 254 | 3006.08 | 1352.97 | 1343.94 | 2.22 | 2.24 |
| N187M | 262 | 4934.44 | 1811.35 | 1793.14 | 2.72 | 2.75 |
| N187K | 253 | 4182.49 | 2425.34 | 2415.57 | 1.72 | 1.73 |
| R195V | 284 | 4847.81 | 2724.92 | 2517.49 | 1.78 | 1.93 |
| A198L | 305 | 6756.76 | 2056.50 | 2046.15 | 3.29 | 3.30 |
| A198M | 301 | 3777.50 | 1708.61 | 1725.14 | 2.21 | 2.19 |
| S210V | 341 | 3349.95 | 1249.47 | 1622.57 | 2.68 | 2.06 |
| Y218S | 354 | 2878.50 | 2373.98 | 2187.48 | 1.21 | 1.32 |
| F223E | 365 | 8318.70 | 3685.68 | 5283.08 | 2.26 | 1.57 |
| V227W | 397 | 996.55 | 729.20 | 834.38 | 1.37 | 1.19 |
| L229I | 436 | 2790.27 | 1050.86 | 1738.46 | 2.66 | 1.61 |
| I240C | 483 | 2688.75 | 561.91 | 884.15 | 4.78 | 3.04 |

TABLE 18
Partially Reversible hMMP-1 mutants (2 Hours, 37° C.

| | | | | | | Ratio |
|---|---|---|---|---|---|---|
| | | | | RFU | Ratio | 25° C./ |
| hMMP-1 | SEQ ID | RFU | RFU | 37 to | 25° C./ | 34 to |
| mutation | NO | 25° C. | 37° C. | 25° C. | 37° C. | 25° C. |
| D105A | 39 | 5669.31 | 1336.14 | 1509.52 | 4.24 | 3.76 |
| D105F | 33 | 2980.00 | 818.63 | 1004.23 | 3.64 | 2.97 |
| D105G | 32 | 8821.81 | 4313.40 | 4643.53 | 2.05 | 1.90 |
| D105S | 31 | 9355.63 | 7274.97 | 7453.42 | 1.29 | 1.26 |
| D105T | 29 | 4457.16 | 2220.03 | 2177.84 | 2.01 | 2.05 |
| R150P | 59 | 8750.30 | 2497.86 | 3115.73 | 3.50 | 2.81 |
| G159T | 125 | 6704.95 | 2347.74 | 2530.78 | 2.86 | 2.65 |
| E180Y | 182 | 8557.09 | 6079.36 | 6421.56 | 1.41 | 1.33 |
| E180T | 181 | 7870.99 | 1794.15 | 1824.99 | 4.39 | 4.31 |
| E180F | 185 | 8508.13 | 3975.22 | 3981.79 | 2.14 | 2.14 |
| T185H | 233 | 5593.77 | 2534.15 | 2693.25 | 2.21 | 2.08 |
| T185Q | 238 | 7006.87 | 2642.74 | 2589.77 | 2.65 | 2.71 |
| T185A | 248 | 2474.96 | 707.09 | 730.58 | 3.50 | 3.39 |
| T185E | 232 | 3948.43 | 2091.32 | 2106.55 | 1.89 | 1.87 |
| N187R | 254 | 3006.08 | 1421.87 | 1476.42 | 2.11 | 2.04 |
| N187M | 262 | 4934.44 | 1893.07 | 1998.97 | 2.61 | 2.47 |
| N187K | 253 | 4182.49 | 2652.79 | 2902.79 | 1.58 | 1.44 |
| R195V | 284 | 4847.81 | 2984.10 | 3555.03 | 1.62 | 1.36 |
| A198L | 305 | 6756.76 | 2642.76 | 2540.07 | 2.56 | 2.66 |
| A198M | 301 | 3777.50 | 2155.58 | 2802.78 | 1.75 | 1.35 |
| S210V | 341 | 3349.95 | 2314.86 | 2277.32 | 1.45 | 1.47 |
| Y218S | 354 | 2878.50 | 2350.27 | 2383.67 | 1.22 | 1.21 |
| F223E | 365 | 8318.70 | 6209.93 | 7415.02 | 1.34 | 1.12 |
| V227W | 397 | 996.55 | 787.87 | 850.67 | 1.26 | 1.17 |
| L229I | 436 | 2790.27 | 1803.44 | 2453.07 | 1.55 | 1.14 |
| I240C | 483 | 2688.75 | 853.66 | 872.62 | 3.15 | 3.08 |

TABLE 19
Partially Reversible hMMP-1 mutants (Overnight, 34° C.)

| | | | | | | Ratio |
|---|---|---|---|---|---|---|
| | | | | RFU | Ratio | 25° C./ |
| hMMP-1 | SEQ ID | RFU | RFU | 34 to | 25° C./ | 34 to |
| mutation | NO | 25° C. | 34° C. | 25° C. | 34° C. | 25° C. |
| D105A | 39 | 8466.62 | 1302.84 | 1532.38 | 6.50 | 5.53 |
| D105F | 33 | 6725.59 | 938.60 | 1172.86 | 7.17 | 5.73 |
| D105G | 32 | 8940.06 | 3560.75 | 5314.44 | 2.51 | 1.68 |
| D105S | 31 | 9300.85 | 5584.70 | 9413.56 | 1.67 | 0.99 |
| D105T | 29 | 7910.47 | 1899.25 | 3254.16 | 4.17 | 2.43 |
| R150P | 59 | 9011.11 | 3533.16 | 4443.96 | 2.55 | 2.03 |
| G159T | 125 | 9105.95 | 3210.57 | 4179.05 | 2.84 | 2.18 |
| E180Y | 182 | 9281.77 | 6080.89 | 8570.48 | 1.53 | 1.08 |
| E180T | 181 | 8475.04 | 2585.89 | 3901.87 | 3.28 | 2.17 |
| E180F | 185 | 9360.74 | 5183.25 | 7022.64 | 1.81 | 1.33 |
| T185H | 233 | 8531.85 | 3164.69 | 5520.76 | 2.70 | 1.55 |
| T185Q | 238 | 9044.23 | 3639.00 | 5467.27 | 2.49 | 1.65 |
| T185A | 248 | 6156.97 | 1110.68 | 1585.53 | 5.54 | 3.88 |
| T185E | 232 | 8479.18 | 3868.06 | 4836.97 | 2.19 | 1.75 |
| N187R | 254 | 7593.11 | 2415.63 | 3156.74 | 3.14 | 2.41 |
| N187M | 262 | 8605.76 | 2769.52 | 4008.68 | 3.11 | 2.15 |
| N187K | 253 | 8667.36 | 3458.94 | 5465.35 | 2.51 | 1.59 |
| R195V | 284 | 8634.05 | 4648.03 | 5966.81 | 1.86 | 1.45 |
| A198L | 305 | 8795.36 | 3469.36 | 5027.30 | 2.54 | 1.75 |
| A198M | 301 | 8352.73 | 3215.69 | 4220.51 | 2.60 | 1.98 |
| S210V | 341 | 7104.17 | 2441.96 | 3664.23 | 2.91 | 1.94 |
| Y218S | 354 | 7740.61 | 4057.37 | 5769.79 | 1.91 | 1.34 |
| F223E | 365 | 9650.44 | 4849.58 | 9311.40 | 1.99 | 1.04 |
| V227W | 397 | 3070.92 | 1370.13 | 1632.51 | 2.24 | 1.88 |
| L229I | 436 | 7333.92 | 1832.18 | 4427.24 | 4.00 | 1.66 |
| I240C | 483 | 6170.51 | 1174.96 | 2389.06 | 5.25 | 2.58 |

TABLE 20
Partially Reversible hMMP-1 mutants Overnight, 37° C.

| | | | | | | Ratio |
|---|---|---|---|---|---|---|
| | | | | RFU | Ratio | 25° C./ |
| hMMP-1 | SEQ ID | RFU | RFU | 37 to | 25° C./ | 37 to |
| mutation | NO | 25° C. | 37° C. | 25° C. | 37° C. | 25° C. |
| D105A | 39 | 8466.62 | 1931.17 | 2589.08 | 4.38 | 3.27 |
| D105F | 33 | 6725.59 | 1173.23 | 1759.31 | 5.73 | 3.82 |
| D105G | 32 | 8940.06 | 5390.31 | 7139.57 | 1.66 | 1.25 |
| D105S | 31 | 9300.85 | 8234.95 | 8615.33 | 1.13 | 1.08 |
| D105T | 29 | 7910.47 | 3292.01 | 4482.74 | 2.40 | 1.76 |
| R150P | 59 | 9011.11 | 3559.66 | 5181.30 | 2.53 | 1.74 |
| G159T | 125 | 9105.95 | 3160.07 | 4338.35 | 2.88 | 2.10 |
| E180Y | 182 | 9281.77 | 6894.61 | 8986.47 | 1.35 | 1.03 |
| E180T | 181 | 8475.04 | 2809.15 | 3649.72 | 3.02 | 2.32 |
| E180F | 185 | 9360.74 | 5335.15 | 7183.36 | 1.75 | 1.30 |
| T185H | 233 | 8531.85 | 3515.59 | 6101.91 | 2.43 | 1.40 |
| T185Q | 238 | 9044.23 | 4012.93 | 5623.60 | 2.25 | 1.61 |
| T185A | 248 | 6156.97 | 1059.61 | 1315.46 | 5.81 | 4.68 |
| T185E | 232 | 8479.18 | 3892.33 | 5330.81 | 2.18 | 1.59 |
| N187R | 254 | 7593.11 | 2370.01 | 3425.18 | 3.20 | 2.22 |
| N187M | 262 | 8605.76 | 2720.28 | 4400.27 | 3.16 | 1.96 |
| N187K | 253 | 8667.36 | 3709.62 | 6374.32 | 2.34 | 1.36 |
| R195V | 284 | 8634.05 | 4960.91 | 7212.05 | 1.74 | 1.20 |
| A198L | 305 | 8795.36 | 4181.78 | 5395.22 | 2.10 | 1.63 |
| A198M | 301 | 8352.73 | 3637.79 | 5914.49 | 2.30 | 1.41 |
| S210V | 341 | 7104.17 | 3939.90 | 4626.58 | 1.80 | 1.54 |
| Y218S | 354 | 7740.61 | 4093.29 | 6181.92 | 1.89 | 1.25 |
| F223E | 365 | 9650.44 | 7645.34 | 9149.09 | 1.26 | 1.05 |
| V227W | 397 | 3070.92 | 1456.45 | 1695.81 | 2.11 | 1.81 |
| L229I | 436 | 7333.92 | 3268.93 | 5729.00 | 2.24 | 1.28 |
| I240C | 483 | 6170.51 | 2223.23 | 2050.31 | 2.78 | 3.01 |

C. Results: Non Reversible hMMP-1 Mutants

Thirty eight hMMP-1 mutants were determined to be non reversible. The activity of these mutants at 34° C. or 37° C., which is decreased compared to the activity at 25° C., remained decreased when lowered to 25° C. The results are shown in Tables 21-24 below, which list the activities (in RFUs) and the ratios of the activities. Tables 21 and 22 summarize the results at 34° C. or 37° C., respectively, of the hMMP-1 irreversible mutants under the two hour condition. Tables 23 and 24 summarize the results of reversibility at 34° C. or 37° C., respectively, of the irreversible hMMP-1 mutants under the overnight condition. The results are similar under all reaction conditions, temperature and time. The activity at 34° C. or 37° C. overnight is the same or similar to the activity when incubated at 34° C. or 37° C. for one hour then 25° C. overnight. For example, the activity of D105R at 34° C. is 1407 RFU and its activity at 34° C. then overnight at 25° C. is 1424 RFU (see Table 23, below).

TABLE 21
Non Reversible hMMP-1 mutants (2 Hours, 34° C.)

| | | | | | | Ratio |
|---|---|---|---|---|---|---|
| | | | | RFU | Ratio | 25° C./ |
| hMMP-1 | SEQ ID | RFU | RFU | 34 to | 25° C./ | 34 to |
| mutation | NO | 25° C. | 34° C. | 25° C. | 34° C. | 25° C. |
| L95K | 6 | 4650.42 | 748.29 | 833.29 | 6.21 | 5.58 |
| D105I | 36 | 6832.34 | 780.32 | 908.39 | 8.76 | 7.52 |
| D105L | 38 | 4206.38 | 534.24 | 630.66 | 7.87 | 6.67 |
| D105N | 27 | 8920.05 | 918.13 | 1128.03 | 9.72 | 7.91 |
| D105R | 25 | 2821.20 | 722.46 | 843.19 | 3.90 | 3.35 |
| D105W | 35 | 6663.80 | 1690.93 | 2266.26 | 3.94 | 2.94 |
| D151G | 70 | 1264.62 | 589.27 | 664.86 | 2.15 | 1.90 |
| F155A | 96 | 2824.01 | 779.72 | 735.02 | 3.62 | 3.84 |
| D156K | 100 | 8576.47 | 2210.63 | 2318.28 | 3.88 | 3.70 |
| D156T | 105 | 8727.27 | 2679.17 | 2770.95 | 3.26 | 3.15 |
| D156L | 114 | 2916.24 | 576.84 | 655.46 | 5.06 | 4.45 |
| D156A | 115 | 2299.63 | 533.68 | 635.67 | 4.31 | 3.62 |
| D156W | 111 | 1502.86 | 539.74 | 637.12 | 2.78 | 2.36 |
| D156V | 113 | 1593.06 | 534.71 | 634.83 | 2.98 | 2.51 |
| D156H | 99 | 5387.79 | 698.77 | 784.55 | 7.71 | 6.87 |
| D156R | 101 | 7020.81 | 793.83 | 881.39 | 8.84 | 7.97 |
| G159V | 132 | 4673.44 | 856.78 | 789.92 | 5.45 | 5.92 |
| A176F | 148 | 1609.85 | 654.43 | 633.13 | 2.46 | 2.54 |
| D179N | 160 | 5660.69 | 644.51 | 644.98 | 8.78 | 8.78 |
| D181L | 209 | 2710.97 | 619.39 | 645.65 | 4.38 | 4.20 |
| D181K | 195 | 1130.63 | 625.01 | 609.58 | 1.81 | 1.85 |
| E182T | 219 | 3702.08 | 791.23 | 805.48 | 4.68 | 4.60 |
| E182Q | 218 | 1331.50 | 639.84 | 623.88 | 2.08 | 2.13 |
| T185R | 235 | 2637.31 | 1187.63 | 1158.47 | 2.22 | 2.28 |
| N187F | 261 | 3227.96 | 877.21 | 823.16 | 3.68 | 3.92 |
| N187I | 264 | 4218.55 | 849.11 | 869.19 | 4.97 | 4.85 |
| G206A | 324 | 872.27 | 603.01 | 592.13 | 1.45 | 1.47 |
| G206S | 317 | 932.69 | 492.65 | 507.75 | 1.89 | 1.84 |
| V227C | 388 | 1998.67 | 950.01 | 1115.17 | 2.10 | 1.79 |
| V227E | 384 | 7904.54 | 839.00 | 906.06 | 9.42 | 8.72 |
| Q228P | 420 | 1082.56 | 607.78 | 617.33 | 1.78 | 1.75 |
| L229T | 429 | 1221.05 | 580.15 | 605.83 | 2.10 | 2.02 |
| D233E | 440 | 2195.02 | 1393.95 | 1332.07 | 1.57 | 1.65 |
| I234A | 476 | 2375.42 | 1473.70 | 1456.58 | 1.61 | 1.63 |
| I234T | 467 | 1199.18 | 713.83 | 775.40 | 1.68 | 1.55 |
| I234E | 460 | 3920.02 | 705.86 | 829.15 | 5.55 | 4.73 |
| I249S | 488 | 3867.71 | 973.97 | 1027.84 | 3.97 | 3.76 |

TABLE 22
Non Reversible hMMP-1 mutants (2 Hours, 37° C.)

| | | | | | | Ratio |
|---|---|---|---|---|---|---|
| | | | | RFU | Ratio | 25° C./ |
| hMMP-1 | SEQ ID | RFU | RFU | 37 to | 25° C./ | 37 to |
| mutation | NO | 25° C. | 37° C. | 25° C. | 37° C. | 25° C. |
| L95K | 6 | 4650.42 | 746.89 | 1092.61 | 6.23 | 4.26 |
| D105I | 36 | 6832.34 | 1110.07 | 1104.96 | 6.15 | 6.18 |
| D105L | 38 | 4206.38 | 607.46 | 624.88 | 6.92 | 6.73 |
| D105N | 27 | 8920.05 | 1727.44 | 1820.97 | 5.16 | 4.90 |
| D105R | 25 | 2821.20 | 813.68 | 846.09 | 3.47 | 3.33 |
| D105W | 35 | 6663.80 | 3081.59 | 3123.49 | 2.16 | 2.13 |
| D151G | 70 | 1264.62 | 616.51 | 628.65 | 2.05 | 2.01 |
| F155A | 96 | 2824.01 | 746.59 | 867.76 | 3.78 | 3.25 |
| D156K | 100 | 8576.47 | 2310.30 | 2080.22 | 3.71 | 4.12 |
| D156T | 105 | 8727.27 | 2752.35 | 2251.21 | 3.17 | 3.88 |
| D156L | 114 | 2916.24 | 688.08 | 652.06 | 4.24 | 4.47 |
| D156A | 115 | 2299.63 | 554.21 | 606.45 | 4.15 | 3.79 |
| D156W | 111 | 1502.86 | 575.12 | 582.43 | 2.61 | 2.58 |
| D156V | 113 | 1593.06 | 542.36 | 544.49 | 2.94 | 2.93 |
| D156H | 99 | 5387.79 | 819.82 | 881.23 | 6.57 | 6.11 |
| D156R | 101 | 7020.81 | 872.40 | 944.17 | 8.05 | 7.44 |
| G159V | 132 | 4673.44 | 838.46 | 932.14 | 5.57 | 5.01 |
| A176F | 148 | 1609.85 | 618.72 | 741.21 | 2.60 | 2.17 |
| D179N | 160 | 5660.69 | 656.31 | 636.18 | 8.63 | 8.90 |
| D181L | 209 | 2710.97 | 611.92 | 668.31 | 4.43 | 4.06 |
| D181K | 195 | 1130.63 | 608.68 | 646.77 | 1.86 | 1.75 |
| E182T | 219 | 3702.08 | 826.28 | 746.25 | 4.48 | 4.96 |
| E182Q | 218 | 1331.50 | 623.11 | 629.01 | 2.14 | 2.12 |
| T185R | 235 | 2637.31 | 1183.37 | 1158.87 | 2.23 | 2.28 |
| N187F | 261 | 3227.96 | 931.04 | 856.03 | 3.47 | 3.77 |
| N187I | 264 | 4218.55 | 887.80 | 879.78 | 4.75 | 4.80 |
| G206A | 324 | 872.27 | 586.57 | 654.37 | 1.49 | 1.33 |
| G206S | 317 | 932.69 | 463.60 | 552.97 | 2.01 | 1.69 |
| V227C | 388 | 1998.67 | 992.19 | 1130.51 | 2.01 | 1.77 |
| V227E | 384 | 7904.54 | 1015.12 | 1127.74 | 7.79 | 7.01 |
| Q228P | 420 | 1082.56 | 586.63 | 777.28 | 1.85 | 1.39 |
| L229T | 429 | 1221.05 | 564.49 | 747.87 | 2.16 | 1.63 |
| D233E | 440 | 2195.02 | 1454.71 | 1976.42 | 1.51 | 1.11 |
| I234A | 476 | 2375.42 | 1594.08 | 1460.23 | 1.49 | 1.63 |
| I234T | 467 | 1199.18 | 796.81 | 833.55 | 1.50 | 1.44 |
| I234E | 460 | 3920.02 | 923.57 | 867.78 | 4.24 | 4.52 |
| I240S | 488 | 3867.71 | 1575.05 | 1594.10 | 2.46 | 2.43 |

TABLE 23
Non Reversible hMMP-1 mutants (Overnight, 34° C.)

| | | | | | | Ratio |
|---|---|---|---|---|---|---|
| | | | | RFU | Ratio | 25° C./ |
| hMMP-1 | SEQ ID | RFU | RFU | 34 to | 25° C./ | 34 to |
| mutation | NO | 25° C. | 34° C. | 25° C. | 34° C. | 25° C. |
| L95K | 6 | 7744.34 | 1803.12 | 1892.59 | 4.29 | 4.09 |
| D105I | 36 | 8394.32 | 1614.57 | 1736.52 | 5.20 | 4.83 |
| D105L | 38 | 6546.78 | 957.95 | 988.23 | 6.83 | 6.62 |
| D105N | 27 | 9119.04 | 1459.16 | 1822.40 | 6.25 | 5.00 |
| D105R | 25 | 5775.25 | 1407.06 | 1424.59 | 4.10 | 4.05 |
| D105W | 35 | 8617.36 | 2851.22 | 4709.94 | 3.02 | 1.83 |
| D151G | 70 | 1956.65 | 959.80 | 1013.03 | 2.04 | 1.93 |
| F155A | 96 | 4891.89 | 2016.76 | 1493.70 | 2.43 | 3.28 |
| D156K | 100 | 8696.27 | 3968.92 | 4371.25 | 2.19 | 1.99 |
| D156T | 105 | 8972.20 | 3971.43 | 4480.62 | 2.26 | 2.00 |
| D156L | 114 | 5254.55 | 972.64 | 1011.27 | 5.40 | 5.20 |
| D156A | 115 | 3585.37 | 1098.25 | 1057.84 | 3.26 | 3.39 |
| D156W | 111 | 2570.24 | 1091.27 | 1126.01 | 2.36 | 2.28 |
| D156V | 113 | 2208.99 | 954.21 | 954.54 | 2.31 | 2.31 |
| D156H | 99 | 7587.19 | 1451.49 | 1440.25 | 5.23 | 5.27 |
| D156R | 101 | 8622.23 | 1735.02 | 1760.60 | 4.97 | 4.90 |
| G159V | 132 | 6555.27 | 1821.53 | 1524.05 | 3.60 | 4.30 |
| A176F | 148 | 4191.69 | 1414.21 | 1181.99 | 2.96 | 3.55 |
| D179N | 160 | 7317.57 | 1504.84 | 1458.70 | 4.86 | 5.02 |
| D181L | 209 | 4534.34 | 1078.98 | 984.43 | 4.20 | 4.61 |
| D181K | 195 | 1869.47 | 946.27 | 841.77 | 1.98 | 2.22 |
| E182T | 219 | 6752.25 | 1483.52 | 1570.77 | 4.55 | 4.30 |
| E182Q | 218 | 2212.75 | 1065.07 | 929.49 | 2.08 | 2.38 |
| T185R | 235 | 6281.97 | 2425.71 | 2808.30 | 2.59 | 2.24 |
| N187F | 261 | 7352.85 | 1612.23 | 1533.32 | 4.56 | 4.80 |
| N187I | 264 | 8306.40 | 1459.25 | 1598.90 | 5.69 | 5.20 |
| G206A | 324 | 2492.53 | 1038.14 | 906.63 | 2.40 | 2.75 |
| G206S | 317 | 2845.84 | 908.82 | 816.00 | 3.13 | 3.49 |
| V227C | 388 | 5833.84 | 2207.20 | 2739.65 | 2.64 | 2.13 |
| V227E | 384 | 8630.90 | 2283.07 | 2096.30 | 3.78 | 4.12 |
| Q228P | 420 | 3673.33 | 1162.95 | 1213.48 | 3.16 | 3.03 |
| L229T | 429 | 3543.75 | 1103.34 | 1105.90 | 3.21 | 3.20 |
| D233E | 440 | 6694.93 | 2570.71 | 3171.20 | 2.60 | 2.11 |
| I234A | 476 | 6250.56 | 3890.90 | 3608.10 | 1.61 | 1.73 |
| I234T | 467 | 3507.08 | 1099.58 | 1194.99 | 3.19 | 2.93 |
| I234E | 460 | 7541.73 | 1365.08 | 1817.16 | 5.52 | 4.15 |
| I240S | 488 | 4376.99 | 2108.15 | 2290.56 | 2.08 | 1.91 |

TABLE 24
Non Reversible hMMP-1 mutants (Overnight, 37° C.)

| | | | | | | Ratio |
|---|---|---|---|---|---|---|
| | | | | RFU | Ratio | 25° C./ |
| hMMP-1 | SEQ ID | RFU | RFU | 37 to | 25° C./ | 37 to |
| mutation | NO | 25° C. | 37° C. | 25° C. | 37° C. | 25° C. |
| L95K | 6 | 7744.34 | 1677.96 | 2463.18 | 4.62 | 3.14 |
| D105I | 36 | 8394.32 | 1958.96 | 1925.73 | 4.29 | 4.36 |
| D105L | 38 | 6546.78 | 1070.51 | 939.53 | 6.12 | 6.97 |
| D105N | 27 | 9119.04 | 2347.74 | 2813.87 | 3.88 | 3.24 |
| D105R | 25 | 5775.25 | 1499.57 | 1312.01 | 3.85 | 4.40 |
| D105W | 35 | 8617.36 | 4593.06 | 5698.08 | 1.88 | 1.51 |
| D151G | 70 | 1956.65 | 1097.68 | 900.59 | 1.78 | 2.17 |
| F155A | 96 | 4891.89 | 1843.31 | 1882.95 | 2.65 | 2.60 |
| D156K | 100 | 8696.27 | 3858.90 | 4126.13 | 2.25 | 2.11 |
| D156T | 105 | 8972.20 | 3854.84 | 3990.29 | 2.33 | 2.25 |
| D156L | 114 | 5254.55 | 1232.94 | 1008.08 | 4.26 | 5.21 |
| D156A | 115 | 3585.37 | 1110.73 | 940.62 | 3.23 | 3.81 |
| D156W | 111 | 2570.24 | 1206.22 | 997.15 | 2.13 | 2.58 |
| D156V | 113 | 2208.99 | 997.64 | 777.35 | 2.21 | 2.84 |
| D156H | 99 | 7587.19 | 1763.27 | 1536.01 | 4.30 | 4.94 |
| D156R | 101 | 8622.23 | 1846.71 | 1764.13 | 4.67 | 4.89 |
| G159V | 132 | 6555.27 | 1683.20 | 1842.91 | 3.89 | 3.56 |
| A176F | 148 | 4191.69 | 1336.32 | 1553.01 | 3.14 | 2.70 |
| D179N | 160 | 7317.57 | 1485.28 | 1378.59 | 4.93 | 5.31 |
| D181L | 209 | 4534.34 | 1000.80 | 1020.08 | 4.53 | 4.45 |
| D181K | 195 | 1869.47 | 928.55 | 895.45 | 2.01 | 2.09 |
| E182T | 219 | 6752.25 | 1496.55 | 1319.53 | 4.51 | 5.12 |
| E182Q | 218 | 2212.75 | 1035.24 | 916.32 | 2.14 | 2.41 |
| T185R | 235 | 6281.97 | 2300.61 | 2829.34 | 2.73 | 2.22 |
| N187F | 261 | 7352.85 | 1704.23 | 1533.08 | 4.31 | 4.80 |
| N187I | 264 | 8306.40 | 1465.77 | 1560.83 | 5.67 | 5.32 |
| G206A | 324 | 2492.53 | 974.96 | 1057.32 | 2.56 | 2.36 |
| G206S | 317 | 2845.84 | 808.42 | 908.44 | 3.52 | 3.13 |
| V227C | 388 | 5833.84 | 2432.82 | 2707.71 | 2.40 | 2.15 |
| V227E | 384 | 8630.90 | 2152.81 | 2615.26 | 4.01 | 3.30 |
| Q228P | 420 | 3673.33 | 1081.32 | 1681.57 | 3.40 | 2.18 |
| L229T | 429 | 3543.75 | 1030.05 | 1488.58 | 3.44 | 2.38 |
| D233E | 440 | 6694.93 | 2661.43 | 4531.45 | 2.52 | 1.48 |
| I234A | 476 | 6250.56 | 4043.80 | 3433.03 | 1.55 | 1.82 |
| I234T | 467 | 3507.08 | 1228.23 | 1397.18 | 2.86 | 2.51 |
| I234E | 460 | 7541.73 | 1901.96 | 1783.16 | 3.97 | 4.23 |
| I240S | 488 | 4376.99 | 2592.19 | 3417.53 | 1.69 | 1.28 |

### Example 5

### Proteolytic Activity of hMMP-1 on Insoluble Collagen

In this example, the collagenase activity of hMMP-1 was assessed for the protein substrate collagen using SDS-PAGE analysis. Wildtype hMMP-1 cleaves insoluble collagen (α1(I) and α2(I) chains) into three-quarter and one-quarter length digestion products. In this assay, a fluorescein isothiocyanate (FITC)-conjugated collagen was used as the substrate and the reaction was monitored by SDS-PAGE of the reaction products. Cleavage of α1(I) and α2(I) collagen chains results in ¾ and ¼ length digestion products which are distinguishable from full length collagen by separation on SDS polyacrylamide gels. Alternatively, cleavage was assessed by fluorometric analysis. A similar assay can be used to assess the activity of mutant hMMPs for cleavage activity at 25° C. versus 34° C. or 37° C.

A. SDS-PAGE Analysis

In this Example, wild-type MMP-1 was tested for cleavage of insoluble collagen and assessed by SDS-PAGE. In short, 2 μg of hMMP-1 (purchased from R&D Systems, #901-MP; or BAP006_{—}2 and BAP006_{—}10 purified as described in Example 1.B) was diluted in TCNB containing 1 mM AMPA and incubated at the reaction temperature (25° C. or 37° C.) for 2 hours. This activation step cleaves the pro-peptide and generates mature hMMP-1. Subsequently, 6 μg of insoluble collagen conjugated to fluorescein isothiocyanate (FITC) (Anaspec #85111 or Sigma Collagen #C4361) in 20 μl TCNB was added to each activated hMMP-1 aliquot and the mixture was incubated at 25° C. or 37° C. for 24 hours or 6 days.

Cleavage of the insoluble collagen was observed by SDS/PAGE. The reaction mixture was separated on a 7.5% SDS polyacrylamide gel and visualized by staining with Coomassie Blue dye. SDS/PAGE results show that after 24 hours incubation at 25° C. or 37° C., hMMP-1 partially cleaved the α1(I) and α2(I) collagen chains into ¾ and ¼ length digestion products for all hMMP-1 proteins tested. After 6 days at 25° C., complete cleavage into ¾ and ¼ length digestion products was observed. After 6 days at 37° C., the collagen was digested completely. The ¾ and ¼ length collagen digestion products are thermally unstable at body temperature.

B. Fluorometric Analysis

Alternatively, collagenase activity was measured using a fluorescence assay. 5 μg hMMP-1 (purchased from R&D Systems, #901-MP; or BAP006_{—}2 and BAP006_{—}10 purified as described in Example 1.B) was diluted in TCNB containing 1 mM AMPA to a final concentration and incubated at 37° C. for 2 hours. The activity of hMMP-1 for FITC-labeled collagen (Sigma #C4361 or Elastin #CF308) was assessed using a protocol adapted from Baici A et al. (1980) Anal. Biochem., 108: 230-232). Briefly, hMMP-1 was incubated with the substrate for 144 hours at 37° C. As a negative control, the substrate was incubated with buffer only. Following incubation, the reaction mixture was first centrifuged to remove insoluble particles. Fluorescence of the supernatant was detected by measuring fluorescence in a fluorescent plate reader at 495 nm excitation/520 nm emission. Relative fluorescence units (RFU) were determined. Duplicate reactions were performed for each sample.

The results (see Tables 25 and 26 below) show that incubation of insoluble collagen with wildtype hMMP-1 at 37° C. for 144 hours resulted in cleavage of collagen as indicated by high RFU values compared to buffer only control. For example, for cleavage of collagen from Sigma, all hMMPs tested had an RFU between about 1000.00-1200.00 compared to buffer only with an RFU value of about 400.00. The activity of purified collagens from CHO-S (BAP006_{—}2) and BL21 cells (BAP006_{—}10) for cleavage of Sigma insoluble collagen was comparable to hMMP-1 purchased from R&D systems. For cleavage of Elastin collagen, the activity of recombinant hMMP-1 purchased from R&D and BAP006_{—}10 were about 3000.00 RFU, while the activity of BAP006_{—}2 was about 2000.00 RFU. Buffer only exhibited a background fluorescence for cleavage of Elastin collagen of about 1500.00 RFU.

TABLE 25
Cleavage of Collagen (Sigma Insoluble Substrate)

| hMMP-1 | 37° C. | 37° C. | Avg 37° C. | St Dev |
|---|---|---|---|---|
| R&D systems | 1163.17 | 1137.81 | 1150.49 | 17.93 |
| Buffer only | 481.49 | 490.57 | 486.03 | 6.42 |
| BAP006_2 (CHO) | 1265.61 | 1275.17 | 1270.39 | 6.76 |
| BAP006_10 (BL21) | 1292.36 | 1335.14 | 1313.75 | 30.25 |

TABLE 26
Cleavage of Collagen (Elastin Insoluble Substrate

| hMMP-l | 37° C. | 37° C. | Avg 37° C. | St Dev |
|---|---|---|---|---|
| R&D systems | 3488.224 | 2981.417 | 3235.32 | 357.66 |
| Buffer only | 1312.511 | 1807.479 | 1560.00 | 350.00 |
| BAP006_2 (CHO) | 1729.757 | 2297.573 | 2013.67 | 401.51 |
| BAP006_10 (BL21) | 2669.758 | 3056.381 | 2863.07 | 273.38 |

### Example 6

### Identification of Temperature Sensitive Mutants in the Hemopexin Binding Domain

A hMMP-1 mutant library was generated similar to Example 1 by introducing mutations in the parent human MMP-1 DNA to generate single amino acid variants of MMP- in the hemopexin domain at amino acid positions 259, 260, 261, 262, 263, 264, 301, 302, 303, 304, 305, 306, 441, 442, 443, 444, 445 and 446. The mutants were expressed as described in Example 1 and tested for enzymatic activity against a fluorogenic peptide substrate as described in Example 2. One mutant, C259Q (set forth in SEQ ID NO:3532), was identified as a hit with increased activity at 25° C. compared to 37° C.

Next, 11 double mutants were generated containing C259Q and one of L95K; D105N; R150P; D156K; D156T; G159V; D179N; E180T; A198L; V227E or I240S. These double mutants were expressed as described in Example 1 and tested for enzymatic activity against a fluoroaenic pttide substrate as described in Example 2. Five (5) double mutants were identified that were active at 25° C. but show decreased activity at 37° C. The identified double mutants were C259Q/D105N (SEQ ID NO:3533); C259Q/R150P (SEQ ID NO:3534); C259Q/G159V (SEQ ID NO:3535); C259Q/D179N (SEQ ID NO:3536); and C259Q/E180T (SEQ ID NO:3537). The mutants exhibited a ratio of activity (25° C./37° C.) of 10-fold to almost 25-fold, with the C259Q/D179N exhibiting the greatest ratio of activity at almost 25-fold.

### Example 7

### Generation of Activity & Temperature-Sensitive Combination Mutants

Three (3) hMMP-1 variant activity mutants (S208K set forth in SEQ ID NO:3538; I213G set forth in SEQ ID NO:3539; and G214E set forth in SEQ ID NO:3540), identified in Table 9 as having higher activity at 37° C. and 25° C., were used to generate double mutants with the temperature-sensitive hits set forth in Table 14. Each activity mutant was combined with each of the 11 temperature-sensitive hits set forth in Table 14 (**) to generate double mutants. Wildtype hMMP-1 and 31 double mutants were transformed into E. coli BL21 (DE3) competent cells in 14 mL tubes as described in Example 1. Protein was expressed as described in Example 1 upon the addition of 1 mM IPTG at 25° C. Cells were collected 6 hours post-induction. Periplasmic proteins were prepared by incubating the cells in OS buffer (200 mM Tris-HCl, pH 7.5, 20% sucrose, 1 mM EDTA) with DNAse, RNAse and lysozyme. After addition of H₂O to the cells in OS buffer, the cells were centrifuged. The supernatants which contain the periplasmic fractions were transferred to another tube. Supernatants were used to measure the proteolytic activity of hMMP-1 produced by BL21 cells transformed with the wildtype and the double mutants using the assay described in Example 2. The supernatants were incubated with APMA at 37° C. and 25° C. to activate the enzymes. Fluorogenic peptide IX was used as the substrate to determine the activity of hMMP-1. Fluorescence was measured using wavelengths of 320 nm (excitation) and 405 nm (emission) with a microtiter plate fluorescence reader. Duplicate reactions were done for each sample. The ratios were determined by dividing the activities at 25° C. to the activities at 37° C. The value of background activities were substrated from the activities of the wildtype and double mutants. The results showed that incorporation of the activity mutation did not increase the activity of the temperature-sensitive mutants at 25° C. Six (6) double mutants, however, were identified as exhibiting activity at 25° C., but show decreased activity at 37° C. These double mutants include: S208K/G159V (SEQ ID NO:3541); S208K/D179N (SEQ ID NO:3542); S208K/V227E (SEQ ID NO:3543); G214E/G159V (SEQ ID NO:3544); G214E/D179N (SEQ ID NO:3545); and I213G/D179N (SEQ ID NO:3546). The ratio of activity (25° C./37° C.) of the mutants were as follows: almost 14-fold for the S208K/G159V mutant; about 14-fold for the S208K/D179N mutant; about 13-fold for the S208K/C227E mutant; about 8-fold for the G214E/G159V mutant; almost 14-fold for the G214E/D179N mutant; and about 14-fold for the I213G/D179N mutant. As expected, wild-type hMMP-1 exhibited a ratio of activity of about 1-fold.

### Example 8

### Proteolytic Activity of hMMP-1 Variants on Collagens

Cleavage activity of wild-type and various mutant hMMP-1's for Collagen Type I and Type IV at 25° C. or 37° C. was tested by separation on SDS polyacrylamide gels and analysis of digestion products. Wild-type hMMP-1 used in these experiments included mammalian expressed purchased from R&D systems (R&D Systems, Catalog #901-MP; NSO cells) or E. coli expressed (BL21 cells) as described in Example 1B. hMMP-1 variants were expressed in E. coli BL21 cells as described in Example 1A, and E. coli supernatant lysates were further purified using Q-Fast Flow Resin (GE Healthcare) to remove some contaminating proteins as described in Example 1.

Briefly, 0.025 mL of wildtype hMMP-1 or hMMP-1 TS variant E. coli lysates were diluted into 0.175 mL TCNB buffer containing 1 mM APMA. The preparations were incubated for 2 hours at 25° C. to activate the MMP. Activation was confirmed by Western Blot, by a downward shift in MMP-1 molecular weight. The activated preparation was divided into 0.1 mL aliquots, then pre-incubated for another 2 hours at either 25° C. or 37° C. prior to addition to purified soluble or insoluble collagens. Then, 20 μg soluble Human Collagen Type I (BD Biosciences), 10 μg soluble Human Collagen Type IV (Millipore) after lyophilization to remove acetic acid, or 30 μg pH neutralized Gelled-Insoluble Rat Collagen Type I (BD Biosciences) were incubated in the presence of the activated and preincubated wildtype or variant hMMP-1's for 24 hours at 25° C. Digestion products were analyzed by SDS-PAGE. The results are depicted in Table 27. A (+) indicates that digestion products were present, while a (−) indicates that no digestion product of the collagen was observed. The results show that, as expected, each of the wildtype hMMP-1 tested digested Collagen I (both soluble and insoluble) whether preincubated at 25° C. or 37° C. In contrast, for the hMMP-1 variants, digestion products of collagen I were observed from both gelled and lyophilized collagen I, only when the variants were preincubated at 25° C. prior to exposure to collagen I. No collagen I digestion was observed, after 37° C. pre-incubation of the hMMP-1 variants. No Collagen IV digestion products were detected, confirming that, like wildtype hMMP-1, the variant hMMP-1's do not cleave collagen IV.

TABLE 27
MMP-1 Digestion of Purified Collagens

| | R&D | | | | | | |
|---|---|---|---|---|---|---|---|
| | Sys- | E.coli- | | | | | |
| | tems | ex- | | | | | |
| Pre- | WT | pressed | | | | | |
| incu- | MMP- | WT | | | | | |
| bation | 1 | MMP-1 | D179N | G159V | S208K/G159V | D156R/D179N | V227E |

Digestion of Collagen Type I Lyophilized

| | | | | | | | |
|---|---|---|---|---|---|---|---|
| 25° C. | + | + | + | + | + | + | + |
| 37° C. | + | + | − | − | − | − | − |

Digestion of Collagen Type IV Lyophilized

| | | | | | | | |
|---|---|---|---|---|---|---|---|
| 25° C. | − | − | − | − | − | − | − |
| 37° C. | − | − | − | − | − | − | − |

Digestion of Collagen Type I Gels

| | | | | | | | |
|---|---|---|---|---|---|---|---|
| 25° C. | + | + | + | + | + | + | + |
| 37° C. | + | + | − | − | − | − | − |

### Example 9

### Kinetic Assay of hMMP-Variant Enzymatic Activity

Activity of wildtype or variant hMMP-1's expressed from E. coli lysates (Example 1) or enriched by Q-Fast Flow Resin (GE Healthcare) to remove some contaminating proteins (Example 11) was measured in a kinetic assay for cleavage of its substrate from the linear portion of the kinetic curve. Wildtype MMP-1 purchased from AnaSpec also was tested (catalog No. 72004).

Briefly, 0.01 mL of wildtype or variant hMMP-1's were diluted into 0.19 mL TCNB buffer containing 1 mM APMA. The preparations were incubated for 2 hours at 25° C. to activate the MMP. The preparations were then split (into two 100 μl aliquots) and pre-incubated at either 25° C. or 37° C. for 2 hours. Then, activated and pre-incubated hMMP-1 samples were added to a 96-well microplate to which Mca-K-P-L-G-L-Dpa-A-R—NH₂ fluorescent substrate was added to wells of the microplate.

Kinetic analysis of enzymatic activity was performed in a SpectraMax® fluorescent microplate reader at 25° C. Readings were taken once every ˜23 seconds from 0 to 3600 seconds (1 hour), and analyzed using Softmax® Pro Software (Molecular Devices). Based on the extended substrate digestion times monitored for the amount of substrate added to wells, the maximal processable substrate observed to be released is about 17000 RFU. The half maximal substrate processed (about 8500 RFU), by the fastest enzyme, released the 8500 RFU after 500 seconds into the reaction; therefore, the timepoint of 500 seconds was used as endpoint to determine Vmax, just before half substrate was used. The maximum slope of the kinetic display of relative fluorescence units released versus time was calculated with SOFTmax PRO software and is reported as Vₘₐₓ units per second. Vmax units per second values at the 500 sec time point were used as end points for sample comparisons, which, as described above, is the timepoint where less than 50% of the substrate was utilized in the assay by all samples tested. Thus, the substrate has not become limiting in any well assayed. Higher Vₘₐₓ values correspond to an increased presence of the processed substrate.

Table 28 sets forth the results of the analysis for hMMP-1 and variants produced in E. coli lysates or Q-Ft Enriched E. coli lysates. The kinetic results confirm the temperature-sensitivity of the variants at 25° C. as measured by end-point methods for screening.

TABLE 28
Kinetic Assay

| | | | | Q-FT Enriched | | |
|---|---|---|---|---|---|---|
| | E. coli Lysates | | | Vmax per second | | |
| | Vmax per second | | Ratio | | | Ratio |
| | 25° C. | 37° C. | (25/37) | 25° C. | 37° C. | (25/37) |
| Ananspec | 7.208 | 8.879 | 0.8 | 8.042 | 9.177 | 0.9 |
| wildtype | 13.000 | 10.621 | 1.2 | 12.304 | 10.145 | 1.2 |
| D179N | 3.319 | 0.262 | 12.7 | 1.598 | 0.087 | 18.4 |
| G159V | 0.611 | 0.026 | 23.5 | 5.629 | 0.468 | 12.0 |
| S208K/G159V | 0.392 | 0.011 | 35.6 | 4.729 | 0.187 | 25.3 |
| D156T/D179N | 0.662 | 0.116 | 5.7 | 1.439 | 0.039 | 36.9 |
| V227E | 0.846 | 0.087 | 9.7 | 1.309 | 0.595 | 2.2 |

### Example 10

### Comparison of Expression and Specific Activity of hMMP-1 Variants with or without His Tag

hMMP-1 mutants were expressed in E. coli without a His tag using the pET base vector described in Example 1 A.1. The proteins were expressed in E. coli BL21 cells as described in Example 1 A.1. Expression of each mutant was assessed from Western blot analysis of periplasmic extracts of BL21 cells transformed with the constructs using a primary goat anti-hMMP1 antibody (R&D System) followed by detection with a secondary HRP-anti-goat IgG antibody (CalBioChem). The expression levels of each mutant with or without a His tag was normalized by dividing the value of their expression level by the value of the expression level of the wildtype hMMP-1 without a His tag. The normalized expression level of wild type hMMP-1 without a His tag is 1. The normalized expression level of the other tested proteins is set forth in Table 29.

TABLE 29
Normalized Expression Level

| Without a His tag | | With a His tag | |
|---|---|---|---|
| | Normalized to | | Normalized |
| Variant | wildtype | Variant | to wildtype |
| Wildtype | 1.00 | Wildtype-his | 0.51 |
| D105N | 0.57 | D105N-his | 0.77 |
| R1S0P | 0.39 | R150P-his | 0.46 |
| D156K | 0.68 | D156K-his | 2.52 |
| D156T | 0.66 | D156T-his | 0.75 |
| G159V | 0.30 | G159V-his | 0.11 |
| D179N | 1.21 | D179N-his | 1.22 |
| E180T | 0.98 | E180T-his | 1.05 |
| A198L | 0.07 | A198L-his | n/a |
| V227E | 0.20 | V227E-his | 0.04 |
| I240S | −0.01 | 1240S-his | 0.03 |

The normalized expression levels were used to determine the specific activity of the mutants. Activity was assessed similar to Example 2 using a fluorogenic substrate. Each mutant was activated at the indicated temperature (25° C. or 37° C.) added at 25° C. and incubated at the indicated temperature (25° C. or 37° C.) for four hours. Fluorescence was detected by measuring fluorescence in a fluorescent plate reader at 320 nm exitation/405 nm emission. Relative fluorescence units (RFU) were determined. Specific activities at 25° C. and 37° C. was determined by dividing the activities at 25° C. or 37° C. to the normalized expression level. Data was normalized to vector only and background RFU was subtracted. The therapeutic index (TI; ratio of normalized activity at 25° C./37° C.) was determined. The TI of wildtype with or without a His tag was about 1-fold.

The results show that the mutants without a His tag exhibited a TI ranging from almost 5-fold to about 30-fold. For example, the TI of variant D105N was about 5-fold; R150P was almost 5-fold; D156K was about 11-fold; D156K was about 10-fold; G159V was about 16-fold; D179N was about 30-fold; E180T was about 5-fold; A198L was about 10-fold; and V227E was almost 25-fold. The results show that the presence of the His tag had a decreasing effect on some of the mutants activity. For example, the results show that the mutants with a His tag exhibited a TI ranging from just greater than wild-type to about 10-fold. Most mutants with a His tag exhibited a TI that was less than 5-fold. The highest TI observed for the mutants containing a His tag was for D179N-his exhibiting a TI of about 10-fold compared to a TI of D179N without a His tag of about 30-fold.

The percentage of normalized activity of the variant MMPs without a His tag at the indicated temperature (25° C. or 37° C.) was compared to the activity of wildtype hMMP-1 without a His tag. For percentage of activity at 25° C., normalized activities of mutants activated and incubated with substrate at 25° C. were divided by the normalized activity of wildtype MMP-1 activated and incubated with substrate at 25° C. The results show that the mutants D105N, D156T, and E180T exhibited about 120% of the activity of wildtype; mutants G159V, S208K/G159V, V227E exhibited similar activity as wildtype, i.e. about 100% of the activity of wildtype; mutants D156T/D179N, R150P and D156K exhibited about 80% of the activity of wildtype; D179N exhibited about 50% of the activity of wildtype; and mutant D179N/I240S exhibited about 35% of the activity of wildtype.

For the percentage of activity at 37° C., normalized activities of mutants activated at 25° C., preincubated at 37° C. for 2 hours and incubated at 37° C. with substrate were divided by the normalized activity of wildtype MMP-1 activated at 25° C. and incubated with substrate at 25° C. The results show that mutants D179N, S208K/G159V, D156TID179N, and D179N/I240S exhibited less than 5% of the activity of wildtype; mutant G159V exhibitedjust over 5% of the activity of wildtype; mutants V227E, D105N, D156K and D156T exhibited about 10% to about 12% the activity of wildtype; mutants R150P exhibited about 20% the activity of wildtype; and mutant E180T exhibited almost 30% the activity of wildtype.

### Example 11

### 100 mL Scale Expression and Purification of hMMP-1 Mutants with Q-Sepharose Fast Flow Resin

hMMP-1 and variants were purified and enriched from periplasmic preparation using a Q-Sepharose Fast Flow (FF) Resin (GE Healthcare). Briefly, wildtype hMMP-1s and mutants were cloned into pET303CHis to either be expressed with or without a His tag using routine molecular biology techniques. The tested wildtype hMMP-1 included clone BAP006-09 (without a His tag; having a sequence of nucleotides set forth as nucleotides in SEQ ID NO:706 and containing a pel B signal sequence encoding amino acids set forth in SEQ ID NO:3547) and clone BAP006-10 (having a sequence of nucleotides set forth as nucleotides in SEQ ID NO:706 and containing a pel B signal sequence encoding amino acids set forth in SEQ ID NO:3547 and sequence encoding a C-terminal His tag as described in Example 1. B). Plasmids were transformed into BL21 (DE3) E. coli cells and the transformation culture was used to inoculate 15 mL LB medium containing ampicillin additives (in a 50 mL conical) and grown overnight at 37° C. LB without antibiotics was pre-warmed to 37° C. by incubating 100 mL LB medium (in a 500 mL or 100 mL Erlenmeyer flask) overnight. The OD₆₀₀ of the inoculated culture was measured the next morning until the OD₆₀₀ was 0.05-0.1. Ampicillin antibiotics were added to the pre-warmed LB. The 100 mL pre-warmed LB culture with antibiotics was inoculated with the 15 mL overnight culture. The OD₆₀₀ was measured after 60 and 120 minutes, and then every 30 minutes until the OD₆₀₀ reached about 0.6. At OD₆₀₀˜0.6, 1 mL was removed and spun down and periplasmic proteins were prepared as described below for use in analysis. The remaining culture was placed in a 25° C. incubator for 30 minutes (20° C. for combination mutants). The cultures were induced with IPTG at a final concentration of 1 mM and the culture was incubated at 25° C. (or 20° C.) with shaking for 6 hours. After 6 hours, the OD₆₀₀ was measured.

To prepare periplasmic proteins from the 100 mL culture, the induced culture was transferred to 250 mL conicals and the cells were spun down at 1500 g for 10 minutes at room temperature. An enzyme mix was prepared containing 10 mg DNAase and 10 mg lyzozyme dissolved in 1 mL RNAase (10 mg/mL). The mix was filter sterilized and stored at 4° C. Immediately before use, 50 μl of the enzyme mix was added to Buffer I (200 mM Tris/HcL pH7.5, 20% sucrose, 1 mM EDTA). From the cell culture, supernatant was removed and the pellet was carefully resuspended in 2.5 mL Buffer I/enzyme mixture per tube. The mixture was incubated at room temperature for 5 minutes. 2.5 mL of ice cold water tube was added, mixed by inversion, incubated on ice for 10 minutes, and centrifuged at 5000 g for 15 minutes at room temperature to spin down cell debris. Supernatant, containing the hMMP-1 proteins, was combined in a fresh tube as periplasmic proteins and stored in 500 μl aliquots at −20° C. or was purified further using Q Sepharose FF as described below.

Prior to further purifying the protein with Q Sepharose FF, the Q Sepharose FF material was prepared and equilibrated from the original stock by resuspending the contents of the entire bottle and then transferring 10 mL into a 50 mL conical and centrifuging at 4000 g for 3 minutes. The supernatant was discarded and the pellet was resuspended in 20 mL buffer Q-bind (100 mM Tris/HCl, pH 7.5, 10% sucrose, 10 mM CaCl₂, 0.5 mM EDTA). The mixture was centrifuged at 4000 g for 3 minutes and supernatant was removed. This was repeated two times, and after the final spin the pellet was resuspended in 10 mL buffer Q-bind.

To purify the periplasmic prep with Q-Sepharose, 1 mL of the equilibrated Q-Sepharose was centrifuged in a 1.5 mL Eppendorf tube in a microcentrifuge for 2 minutes at full speed. The supernatant was carefully removed. 25 μl 2 M NaCl and 10 μl 1 M CaCl₂ was added to 1 mL periplasmic prep. The 1 mL of perimplasmic prep was used to resuspend the Q-Sepharose, and the mixture was incubated on ice for 10 minutes with occasional mixing. The mixture was centrifuged in a microcentrifuge for 3 minutes at full speed, and the supernatant was transferred to a new tube and saved as “Q-FT 1.” The pellet was resuspended in 1 mL buffer Q bind, and the mixture was centrifuged in a microcentrifuge for 3 minutes at full speed. The supernatant was transferred to a new tube and saved as “Q-FT2.” The pellet was resuspended in 1 mL buffer Q bind, and the mixture was centrifuged in a microcentrifuge for 3 minutes at full speed. The supernatant was transferred to a new tube and saved as “Q-FT3.” The pellet was resuspended in 1 mL buffer Q Elute (100 mM Tris/HCl pH 7.5, 10% sucrose, 10 mM CaCl₂, 1 M NaCl, 0.5 mM EDTA) and the mixture was centrifuged in a microcentrifuge for 3 minutes at full speed. The supernatant was transferred to a new tube and saved as “Q-ET.”

The eluted supernatant was concentrated using an Amicon 30K spin filter (Millipore). The Amicon 30K filter was rinsed with 1 mL Q-bind buffer, and centrifuged at 3000 g in an SW rotor for 5 minutes at room temperature. The buffer was removed from both compartments. 800 μl of the Q-FT1 was added to the filter, and the filter was centrifuged at 3000 g in an SW rotor for 5 minutes at room temperature. The retentate (about 250 μl) was collected.

The various preparations and fractions were analyzed on SDS-PAGE for purity. The activities also were tested following activation by adding 4 μl of lysate, purified Q-FT1, or purified and concentrated Q-FT1 to 96 μl APMA in TCNB. The reaction mixture was incubated for 2 hours at 37° C. or 25° C., followed by the addition of 10 μM fluorescent peptide substrate and incubation for 4 hours at 37° C. or 25° C. In one experiment for the tested proteins (wildtype, D179N, and D156T/D179N), the results show that each of the proteins exhibited activity whether the lysate, purified protein or purified concentrated protein was tested. For wildtype and D179N, the activity of each was substantially the same whether the lysate, purified protein, or purified concentrated protein was tested. For the D156T/D179N double mutant, the activity of the lysate preparation was about half of the activity exhibited by the purified and concentrated preparation. For the Q-Sepharose purified and concentrated preparations, the activity of the D179N mutant and D156T/D179N double mutant at 25° C. was similar to wildtype with an RFU value of about 10,000.00 observed for each condition. The activity of the wildtype was similar at 37° C. or 25° C. In contrast, under all purification conditions tested, the D179N and D156T/D179N mutants exhibited greater activity at 25° C. (about 10,000.00 RFU) than at 37° C. (about 1000 RFU or less), thereby exhibiting greater than 40-fold activity at 25° C. compared to 37° C. Similar results were obtained for other tested mutants (D179N/I240S, G159V, S208K/G159V, V227E, D105N, R150P, D156K, D156T, E180T), with greater activity observed when the tested protein was Q-Sepharose purified compared to when the tested protein was a lysate preparation for many of the proteins tested. Thus, the results show that purification with Q-sepharose in the presence of 10 mM CaCl₂ retains the activities and temperature sensitive phenotype of the mutants.

Purification with Q-Sepharose FF was in the presence of 10 mM CaCl₂. There was no addition of ZnCl₂ in the purification process. If the purification was performed in the absence of 10 mM CaCl₂, the activity of the mutants was reduced.

### Example 12

### Bacterial Expression and Ni-NTA Purification

DNA encoding wildtype hMMP-1 or variants as described in Example 1 were cloned into vector pET-26b containing a C-terminal 6×-His tag (Catalog No. 69862-3, Novagen; SEQ ID NO:3548) at restriction sites NdeI and XhoI. The respective pET26b-hMMP1 vector was transformed into competent BL21(DE3) cells using standard molecular biology techniques and transformants were plated on Kan-LB-agar plates. Two colonies were picked and grown overnight in 50 mL LB media with Kanamycin (50 μg/mL, final concentration) at 37° C. overnight with shaking (200 rpm). For each overnight culture, 20-22 mL of culture was used to inoculate 800 mL of LB media in a 2 L flask (2 baffeled flasks per colony) containing 0.1% glucose, 0.0005% antifoam and 50 μg/mL Kanamycin. The culture was grown at 37° C. with shaking (200 rpm), and the OD₆₀₀ measured. When the OD₆₀₀ reached 0.8-1.3 (about 4.5 hours), the temperature was reduced to 25° C. and IPTG was added to a final concentration of 0.4 mM. Growth was continued overnight (about 12-15 hours) at 25° C. with shaking. Cells were harvested by centrifugation at 4000 g using a JA-5.3 rotor, at 4° C. for 20 minutes for generation of the periplasmic fraction as described below. To confirm protein induction, 1 mL of the culture was centrifuged and resuspended in 200 μl PBS and sonicated to lyse the bacteria. 6×SDS sample buffer containing β-mercaptoethanol (BME) was added to the lysed bacteria, boiled for 10 minutes, and 20 μl was loaded onto a 4-20% TG PAGE Gel. The gel was stained with Simply Blue (Invitrogen) to visualize protein and to determine the degree of protein induction.

For generation of the periplasmic fraction, the harvested cell pellet was re-suspended in 5 mL/gram of lysis buffer (0.5M NaCl, 50 mM Tris-HCL pH 7.9, 10 mM Imidazole, 10% glycerol). To every 40 mL of cell suspension, 1 mM EDTA, 0.5 mg/mL lysozyme, and 50 μl DNase I from a 1 mg/mL stock was added and the suspension was shaken at room temperature for one hour to lyse the bacteria. The cell debris was pelleted by centrifugation at 6000×g at 4° C. for 30 minutes. The supernatant was collected and transferred to new tubes for purification. The pellet was frozen at −80° C. for extraction/solubilization and re-folding of insoluble protein, if desired.

To purify the protein from the supernatant, 5 mL Ni-NTA SuperFlow resin (Qiagen, Cat. No. 30430; 60% slurry) was added to the clarified periplasmic fraction and stirred for 1 hour at 4° C. The mixture was passed through an Econo-column (Biorad) to retain beads and the flow through (FT) and 3 mL bed volume of Ni-NTA resin was collected. The Ni-NTA resin was washed in the column with 3×50 mL of 0.5 M NaCl, 20 mM Tris-HCl pH 7.9, and 10 mM Imidazole. The washes were saved and collected for SDS-PAGE analysis. The MMP-1 was eluted with sequential steps of elution from the column by washing with 6×3 ml of 0.3 M imidazole, 0.5 M NaCl, 20 mM Tris-HCL at pH 7.9, and then 4×3 mL of 1 M imidazole, 0.5 M NaCl, 20 mM Tris-HCl at pH 7.9. The resin was incubated for 5 minutes with each elution step before spinning down the resin. The supernatants after each wash were collected and saved. About 32 μl of each supernatant fraction was run on 4-20% TG PAGE gel as described above to analyze purification efficiency and yield. Also, the proteins were transferred to PVDF membrane using iBlot® (Invitrogen), and Western Blot was performed using goat anti-hMMP1 antibody (R & D Systems, 0.5 μg/mL) and HRP-anti-goat IgG μg/mL).

Based on the overall yield and purity determined by SDS-PAGE analysis, 300 mM imidazole eluents were combined into two pools (typically #2 and 3 and #4-6). Each sample was dialyzed using a 30-KDa Molecular weight cut-off (MWCO) slide-a-lyzer cassette against TCNB buffer in 2 L with one change (2×2 L) at 4° C. overnight. The collected and dialyzed material was stored at 4° C. or −80° C. for longer term storage. The protein concentration was determined by Bradford. For wildtype hMMP-1, typically, about 9-10 mg of protein was purified at about 80% purity obtained from 3.2 L culture.

### Example 13

### Effect of Zinc: Biochemical and Activity Analysis of Enriched MMP-1 Variants

Periplasmic preparations of wildtype hMMP-1 and variants were generated by hypotonic lysis as described in Example 1 with the addition of 3 freeze/thaw/probe sonication steps prior to bacterial debris removal by centrifugation. The clarified bacterial lysate produced with the additional freeze/thaw sonication steps was further purified using Q-Sepharose Fast Flow Resin as described in Example 11. The resulting proteins were further enriched using Mimetic Green I ligand affinity purification bead columns (ProMetic™ Biosciences; Cat. No. A6XL). MMP-1 proteins were eluted from the green mimetic affinity resin, in the presence or absence of 1 mM Zinc, by increasing the NaCl concentration in step elution buffers. Protein was resolved on a 4-20% TG PAGE Gel, and visualized using Simply Blue and by Western Blot.

Activity of the variant MMP-1s purified in the presence of 1 mM Zinc was assessed using a kinetic assay as described in Example 9. Vₘₐₓ units per second values at the 500 sec time point were used as end points for sample comparisons. The results are set forth in Table 30. The results show that there is no temperature sensitivity displayed by the variants when purified in the presence of zinc.

TABLE 30

| | Vmax per second | |
|---|---|---|
| | 25° C. | 37° C. |
| Ananspec | 4.450 | 5.387 |
| wildtype | 1.744 | 1.989 |
| D179N | 3.688 | 3.723 |
| D156T/D179N | 3.996 | 3.972 |
| V227E | 2.332 | 2.216 |

To restore activity, zinc was removed from variants purified in the presence of 1 mM Zinc by chelation with EDTA. P-30 gel filtration spin columns (exclusion 40,000 molecular weight; BioRad) were equilibrated by 4 washes with 0.5 mL 50 mM Tris pH 7.5, 150 mM NaCl, 10 mM CaCl2 and 0.05% Brij35. 0.1 mL of each enriched MMP-1 (purified in the presence of Zn) was mixed with 0.002 mL 500 mM EDTA, pH 8.0 and then loaded onto a buffer-equilibrated spin column. The column was centrifuged for 10-15 seconds at 2000×g and the flow-thru was assayed for MMP-1 activity. By the chelation, the zinc was removed and the NaCl was lowered from 1 M to 150 mM.

To assess activity, 0.01 mL of the flow-thru was added to 0.19 mL TNBC in the presence or absence of 1 mM APMA to activate the protein, and incubated at room temperature (20° C. to 25° C.) for 2 hours. The mixture was split into two with 0.1 mL of the sample removed to a new tube and incubated at 37° C. for 2 hours, with the remaining mixture remaining at room temperature. Then, 0.045 mL TNCB in the presence or absence of 1 mM APMA was added to wells of a 96-well black plate. 0.05 mL of activated/pre-incubated MMP-1 samples at the respective temperature was added to corresponding wells of the assay plate. 0.005 mL of fluorogenic peptide IX substrate was added to each well to initiate the assay. Vₘₐₓ units per second values at the 30 minute time point (1200 sec) were used as end points for sample comparisons. The results are set forth in Table 31. The results show that after EDTA and spin column treatment to chelate zinc, the temperature-sensitivity phenotype was restored.

TABLE 31

| | | | Ratio: |
|---|---|---|---|
| | Vmax per second | | 25° C./ |
| | 25° C. | 37° C. | 37° C. |
| wildtype | 5.401 | 5.115 | 1.05 |
| G159V | 1.309 | −0.003 | 1.3 |
| R150P | 1.514 | 0.171 | 8.8 |
| V227E | 3.268 | 0.773 | 4.2 |
| D179N | 5.569 | 0.143 | 38.9 |
| D156T/D179N | 1.706 | 0.011 | 155.4 |

### Example 14

### Effects of Metals on the Activity of Mutants

In this example, the activity of wild-type MMP-1 and various mutants was tested in the presence of varying concentrations of ZnCl₂, CaCl₂, MgCl₂, and NaCl₂, and the optimal concentration of each for activity determined.

The effect of ZnCl₂ and CaCl₂ was assessed by testing the activity of wild-type MMP-1 and various mutants (D179N, G159V, D156T/D179N) after activating the enzyme by incubation with APMA for 2 hours at 20° C., 25° C. or 37° C. Specifically, 4 μl of periplasmic extract as described in Example 1 was added to 96 μl of APMA in the following solutions: 1) TCNB (50 mM Tris, 10 mM CaCl₂, 150 mM NaCl, 0.05% Brij 3 at pH 7.5); 2) TCNB with 1 mM ZnCl₂; 3) TNB (50 mM Tris, 150 mM NaCl, 0.05% Brij 35 at pH 7.5); or TNB with 1 mM ZnCl₂. After 2 hours, 10 μM fluorogenic peptide IX substrate was added to the reaction mixture and incubated for 4 hours at 20° C., 25° C. or 37° C. Fluorescence was detected by measuring fluorescence in a fluorescent plate reader at 320 nm exitation/405 nm emission. Relative fluorescence units (RFU) were determined. The results show that calcium was required for activity of all of the enzymes, with little to no activity observed under conditions where activation and substrate reaction occurred in TNB buffer. For wild-type MMP-1, the presence of ZnCl₂ slightly reduced activity, suggesting that there was residual zinc present in the periplasmic extracts and/or reaction buffer. For the temperature-sensitive mutants, the presence of 1 mM ZnCl₂ affected the temperature-sensitive phenotypes of the mutants. In the presence of 1 mM ZnCl₂, the ratio of activity at 20° C./37° C. or 25° C./37° C. was dramatically reduced, approaching wild-type levels of about 1.0.

To assess the optimal concentration of ZnCl₂ necessary to retain a temperature-sensitive phenotype, a titration experiment was performed for wild-type MMP-1 and mutant D179N in the presence of 0.001 mM, 0.01 mM, 0.1 mM or 1 mM ZnCl₂. Activity was assessed by adding 4 μl of periplasmic extract to 96 μl APMA in TCNB in the presence or absence of the indicated concentrations of ZnCl₂. The reaction mixture was incubated at 25° C. or 37° C. for 2 hours. After 2 hours, 10 μM fluorogenic peptide IX substrate was added to the reaction mixture and incubated for 4 hours at 25° C. or. Fluorescence was detected by measuring fluorescence in a fluorescent plate reader at 320 nm exitation/405 nm emission, and RFU determined. For wild-type, the results show that activity was substantially the same under all of the tested conditions, with slightly less activity observed at 37° C. than 25° C. Also, at 37° C., activity was slightly lower at 0.1 or 1 mM ZnCl₂ compared to lower concentrations. For the mutant D179N, the activity detected at 25° C. was greatest in the presence of zinc than if zinc was absent (about 9000 RFU in the presence of zinc, compared to about 4000 RFU in the absence of zinc). This activity of the mutant D179N at 25° C. was comparable to wild-type at 25° C., and also was the same in the presence of 0.001 mM, 0.01 mM, or 0.1 mM zinc. The activity of mutant D179N was reduced to about 6000 RFU in the presence of 1 mM zinc. The greatest temperature-sensitive phenotype was observed at 0.001 mM ZnCl₂ (about 13-fold 25° C./37° C. ratio of activity), with decreasing temperature sensitivity detected with increasing concentrations of zinc. In the presence of 0.1 mM and 1 mM ZnCl₂, the D179N exhibited no temperature sensitive phenotype (ratio 25° C./37° C. of about 1.0). Thus, the optimal ZnCl₂ concentration was observed to be at or about 0.001 mM.

A similar experiment was performed to determine the optimal concentration of CaCl₂ necessary to retain activity and a temperature-sensitive phenotype. Activity was assessed by adding 4 μl of periplasmic extract to 96 μl APMA in TCNB in the presence or absence of the indicated concentrations of CaCl₂. The reaction mixture was incubated at 25° C. or 37° C. for 2 hours. After 2 hours, 10 μM fluorogenic peptide IX substrate was added to the reaction mixture and incubated for 4 hours at 25° C. or. Fluorescence was detected by measuring fluorescence in a fluorescent plate reader at 320 nm exitation/405 nm emission, and RFU determined. For wild-type MMP-1. little activity was observed at calcium levels less than 1 mM. Activity was observed at 1 mM CaCl₂, but the activity was greatest at 10 mM CaCl₂ (9000-10,000 RFU). For the D179N MMP-1 variant, activity was only observed in the presence of 10 mM CaCl₂. The activity observed was less than for wild-type, although the sample that was tested was subjected to repeated freezing/thawing, which might affect the activity of the mutant lysate. Thus, the optimal CaCl₂ concentration was observed to be at or about 10 mM or greater than 10 mM.

Similar experiments as above also were performed in the presence or absence of MgCl₂ (0, 0.01 mM, 0.2 mM, 0.2 mM, 1 mM and 10 mM) or NaCl (0, 0.0625 M, 0.125 M, 0.25 M and 0.5M). The results showed that the tested concentrations had no effect on the activities or temperature sensitive phenotypes of the mutants.

Since modifications will be apparent to those of skill in this art, it is intended that this invention be limited only by the scope of the appended claims.

1. A modified matrix metalloprotease-1 (MMP-1), comprising an amino acid replacement in the sequence of amino acid residues of an MMP-1 polypeptide or a catalytically active fragment thereof, wherein:
amino acid replacement confers to the MMP-1 or the catalytically active fragment thereof, a ratio of enzymatic activity at a permissive temperature compared to at a nonpermissive temperature of at least 1.5, wherein:
the permissive temperature is 25° C.;
the non-permissive temperature is 37° C.; and
if the modified MMP-1 is a catalytically active fragment thereof, the active fragment exhibits the ratio of enzymatic activity.

2. (canceled)

3. The modified MMP-1 of claim 1, wherein the unmodified polypeptide comprises the sequence of amino acids set forth in SEQ ID NO: 1, or is an allelic or species variant thereof, a zymogen, a mature form, or a catalytically active fragment that contains the modification.

4. The modified MMP-1 polypeptide of claim 3, wherein the unmodified MMP-1 polypeptide comprises the sequence of amino acids set forth in SEQ ID NO:2, or is an allelic and species variant thereof, a mature form, or a catalytically fragment thereof that contains the modification.

5. The modified MMP-1 polypeptide of claim 3, wherein the catalytically active fragment comprises the catalytic domain or a catalytically active portion of the catalytic domain.

6. A modified MMP-1 of claim 1 that has lower activity at the nonpermissive temperature than the MMP-1 that does not include the modification has at the nonpermissive temperature.

7.-14. (canceled)

15. The modified MMP-1 polypeptide of claim 1 that contains only the catalytic domain of an MMP-1 or a catalytically active portion thereof, wherein the catalytic domain contains at least one of the amino acid replacements that confers the ratio of enzymatic activity.

16. A fusion protein, comprising the modified MMP-1 polypeptide of claim 15 with a second but different polypeptide that is not an MMP-1.

17. The modified MMP-1 polypeptide of claim 1 wherein:
the amino acid replacement is at a position corresponding to any one or more of positions 84, 85, 95, 98, 99, 100, 103, 104, 105, 106, 109, 110, 111, 112, 118, 123, 124, 126, 147, 150, 151, 152, 153, 155, 156, 158, 159, 170, 171, 176, 178, 179, 180, 181, 182, 183, 185, 187, 188, 189, 190, 191, 192, 194, 195, 197, 198, 206, 207, 208, 210, 211, 212, 218, 223, 227, 228, 229, 230, 233, 234, 237, 240, 251, 254, 255, 256, 257 and 258 in an MMP-1 polypeptide comprising the sequence of amino acids set forth in SEQ ID NO:2.

18. A modified MMP-1 polypeptide comprising an amino acid replacement selected from among T84F, E85F, L95K, L95I, R98D, I99Q, E100V, E100R, E100S, E100T, E100F, E100I, E100N, T103Y, P104A, P104M, D105A, D105F, D105G, D105I, D105L, D105N, D105R, D105S, D105T, D105W, D105E, L106C, L106S, A109H, D110A, V111R, D112S, A118T, S123V, N124D, T126S, G147P, R150P, R150V, R150D, R150I, R150H, D151G, N152A, N152S, S153T, F155L, F155A, D156H, D156L, D156A, D156W, D156V, D156K, D156T, D156R, D156M, P158T, P158G, P158K, P158N, G159V, G159T, G159M, G159I, G159W, G159L, G159C, P170D, P170A, G171P, G171E, G171D, A176F, A176W, F178T, F178L, D179N, D179V, D179C, E180Y, E180R, E180T, E180F, E180G, E180S, E180N, E180D, D181T, D181L, D181K, D181C, D181G, E182T, E182Q, E182M, E182G, E183G, R183S, T185R, T185Y, T185H, T185G, T185V, T185Q, T185A, T185E, T185D, N187R, N187M, N187W, N187F, N187K, N187I, N187A, N187G, N187C, N187H, F188V, R189N, R189Q, E190G, E190Y, E190D, Y191V, N192H, N192S, N192D, N192C, H194P, R195C, R195W, R195L, R195G, R195Q, R195A, R195D, R195V, A197V, A197C, A198G, A198L, A198M, G206A, G206S, L207R, L207V, L207I, L207G, S208R, S208L, S210V, S210A, T211L, D212G, D212H, Y218S, F223C, F223E, F223G, F223A, F223S, F223K, F223M, V227C, V227D, V227E, V227L, V227S, V227W, V227G, V227H, V227Q, V227R, Q228P, L229A, L229T, L229I, A230V, D233E, I234A, I234T, I234E, I234Q, I237L, I237W, I237N, I240S, I240A, I240C, I251 S, I251 W, Q254S, T255H, P256C, K257P, K257T and A258P.

19. The modified MMP-1 polypeptide of claim 1 wherein:
the amino acid replacement is at a position corresponding to any one or more of positions 95, 105, 150, 151, 155, 156, 159, 176, 179, 180, 181, 182, 185, 187, 195, 198, 206, 210, 212, 218, 223, 227, 228, 229, 230, 233, 234, and 240 in an MMP-1 polypeptide having a sequence of amino acids set forth in SEQ ID NO:2; and
the amino acid replacement confers to the MMP-1, allelic or species variant thereof or an active fragment thereof, a ratio of enzymatic activity at a permissive temperature compared to at a nonpermissive temperature of at least 1.5.

20. The modified MMP-1 polypeptide of claim 19, wherein the amino acid replacement is selected from among L95K, D105A, D105F, D105G, D105I, D105L, D105N, D105R, D105S, D105T, D105W, R150P, D151G, F155A, D156K, D156T, D156L, D156A, D156W, D156V, D156H, D156R, G159V, G159T, A176F, D179N, E180Y, E180T, E180F, D181L, D181K, E182T, E182Q, T185R, T185H, T185Q, T185A, T185E, N187R, N187M, N187F, N187K, N187I, R195V, A198L, A198M, G206A, G206S, S210V, Y218S, F223E, V227C, V227E, V227W, Q228P, L229T, L229I, D233E, I234A, I234T, I234E, I240S, and I240C.

21. The modified MMP-1 polypeptide of claim 1, wherein the polypeptide retains the activity of the unmodified MMP-1 at the permissive temperature.

22. The modified MMP-1 polypeptide of claim 1, wherein the activity of the polypeptide, following exposure to the nonpermissive temperature, is reversible upon exposure to the permissive temperature.

23. The modified MMP-1 polypeptide of claim 22, wherein the amino acid replacement is selected from among D105A, D105F, D105G, D105S, D105T, R150P, G159T, E180Y, E180T, E180F, T185H, T185Q, T185A, T185E, N187R, N187M, N187K, R195V, A198L, A198M, S210V, Y218S, F223E, V227W, L229I and I240C.

24. The modified MMP-1 polypeptide of claim 1, wherein the activity of the polypeptide is irreversibly inactive upon exposure to the nonpermissive temperature.

25. The modified MMP-1 polypeptide of claim 24, wherein the amino acid replacement is selected from among L95K, D105I, D105L, D105N, D105R, D105W, D151G, F155A, D156K, D156T, D156L, D156A, D156W, D156V, D156H, D156R, G159V, A176F, D179N, D181L, D181K, E182T, E182Q, T185R, N187F, N187I, G206A, G206S, V227C, V227E, Q228E, L229T, D233E, I234A, I234T, I234E and I240S.

26. A modified MMP-1 polypeptide that has a sequence of amino acids set forth in any of SEQ ID NOS: 3-705, 779-3458 3507-3531 and 3541-3546.

27. The modified MMP-1 polypeptide of claim 1, wherein the polypeptide comprises two or more amino acid replacements and the replacements are at a position corresponding to any two or more of positions 95, 105, 150, 156, 159, 179, 180, 182, 185, 187, 198, 227, 234 and 240 in an MMP-1 polypeptide having a sequence of amino acids set forth in SEQ ID NO:2.

28. The modified MMP-1 polypeptide of claim 27, wherein the two or more modifications are selected from among L95K, D105N, R150P, D156K, D156T, G159V, D179N, E180T, A198L, V227E, and I240S.

29. The modified MMP-1 polypeptide of claim 28, wherein the modified MMP-1 polypeptide is selected from among a polypeptide having amino acid replacements D156K/G159V/D179N; R150P/V227E; D156T/V227E; G159V/A198L; D105N/A198L; D179N/V227E; A198L/V227E; E180T/V227E; D179N/A198L; D156K/D179N; D105N/R150P/D156K/G159V/D179N/E180T; D105N/R150P/E180T; G159V/I240S; D156T/D179N/I240S; D156T/G159V; R150P/E180T; D156T/D179N; D179N/I240S; L95K/D156T/D179N; G159V/D179N; L95K/D105N/E180T; R150P/D156T/A198L; L95K/D105N/R150P/D156T/G159V/A198L/V227E/I240S; L95K/R150P; and D105N/E180T.

30. The modified MMP-1 polypeptide of claim 1, further comprising at least one amino acid replacement(s) that confers increased activity compared to the MMP-1 polypeptide not containing the amino acid replacement(s).

31. The modified MMP-1 polypeptide of claim 30, wherein the amino acid replacement(s) is at a position corresponding to any one or more of positions 81, 84, 85, 86, 87, 89, 104, 105, 106, 107, 108, 109, 124, 131, 133, 134, 135, 143, 146, 147, 150, 152, 153, 154, 157, 158, 160, 161, 164, 166, 167, 180, 183, 189, 190, 207, 208, 211, 213, 214, 216, 218, 220, 222, 223, 224, 225, 226, 227, 228, 229, 230, 231, 232, 235, 236, 238, 239, 244, 249, 254, 256, 257 and 258 in an MMP-1 polypeptide comprising the sequence of amino acids set forth in SEQ ID NO:2.

32. The modified MMP-1 polypeptide of claim 31, wherein the amino acid replacement is selected from among F81L, F81A, F81G, F81Q, F81R, F81H, T84H, T84L, T84D, T84R, T84G, T84A, E85S, E85V, G86S, N87P, N87R, N87G, N87Q, R89A, R89T, R89G, R89K, P104E, P104D, P104Q, D105V, L106V, P107T, P107S, P107A, R108E, R108A, R108K, R108S, A109S, A109R, A109G, A109M, A109V, N124G, T131D, K132R, V133T, V133L, S134E, S134D, E135M, S143I, R146S, G147R, G147F, R150E, R150G, R150M, T150T, R150A, R150N, R150K, R150L, R150V, R150D, N152G, N152F, N152L, N152I, S153T, S153P, S153F, S153D, S153Y, P154S, P154I, G157F, P158V, P158I, G160Q, N161L, N161R, N161Y, N161E, N161T, N161I, N161V, N161F, N161Q, H164S, F166W, Q167R, Q167A, Q167S, Q167F, Q167P, Q167T, Q167V, Q167M, E180D, R183S, R189N, R189T, R189Q, E190D, L207M, S208K, S208R, S208L, T211N, I213G, G214L, G214E, L216I, Y218W, S220R, S220A, S220Q, S220T, S220G, S220M, S220V, S220N, T222R, T222P, T222S, T222F, T222N, F223Y, F223H, S224Q, S224K, S224D, G225Q, G225E, G225H, D226S, D226E, D226P, D226I, V227T, Q228A, Q228D, Q228E, Q228G, Q228H, Q228K, Q228L, Q228M, Q228N, Q228R, Q228S, Q228T, Q228W, Q228Y, L229Q, L229P, L229V, A230G, A230W, A230D, A230I, A230S, A230C, A230V, A230T, A230M, A230N, A230H, Q231I, Q231A, Q231F, Q231D, Q231G, Q231V, Q231W, Q231S, Q231H, Q231M, D232H, D232G, D232R, D232P, D232Y, D232S, D232F, D232V, D232K, D232W, D232Q, D232E, D232T, D232L, D235G, D235A, D235L, D235E, D235R, D235Q, D235T, D235N, G236M, G236R, G236S, G236T, G236C, G236K, G236E, G236L, G236N, Q238T, A239S, A239V, A239L, A239I, A239G, A239K, A239H, A239R, S244W, S244Q, Q249W, Q254S, P256S, K257E, K257R, and A258P.

33. The modified MMP-1 polypeptide of claim 32, wherein the modified MMP-1 polypeptide is selected from a polypeptide having amino acid replacements S208K/G159V; S208K/D179N; S208K/V227E; G214E/G159V; G214E/D179N; and I213G/D179N.

34.-35. (canceled)

36. The modified MMP-1 polypeptide of claim 1 that is a zymogen.

37. The modified MMP-1 polypeptide of claim 1 that is a mature enzyme.

38. The modified MMP-1 polypeptide of claim 1 that contains only the catalytically active domain or a catalytically active portion of the catalytic domain.

39. The modified MMP-1 polypeptide of claim 1 that lacks all or a portion of a proline rich linker and/or a hemopexin domain.

40. The modified MMP-1 polypeptide of claim 1 that comprises one or more additional modifications, wherein the one or more additional modifications confer increased stability, increased half-life, altered substrate specificity and/or increased resistance to inhibitors.

41. The modified MMP-1 polypeptide of claim 1 that is glycosylated or PEGylated.

42. The modified MMP-1 polypeptide of claim 1 that is a fusion protein.

43. The modified MMP-1 polypeptide of claim 42 that is fused to an Fc domain or other multimerization domain.

44. A nucleic acid molecule, comprising a sequence of nucleotides encoding a modified MMP polypeptide of claim 1.

45. A vector, comprising the nucleic acid molecule of claim 44.

46. The vector of claim 45, wherein the vector is a prokaryotic vector, viral vector or a eukaryotic vector.

47. The vector of claim 46, wherein the vector is a mammalian vector or a yeast vector.

48. A cell, comprising the vector of claim 45.

49. The cell of claim 48 that is a prokaryotic cell or a mammalian cell.

50. A method of producing a modified MMP-1 polypeptide, comprising:
culturing a cell of claim 48 under conditions whereby the cell expresses the modified MMP-1 polypeptide; and
purifying the MMP-1 polypeptide.

51. A pharmaceutical composition, comprising a modified MMP-polypeptide of claim 1.

52. A method of treating a disease or condition of the extracellular matrix (ECM), comprising administering to the ECM a pharmaceutical composition of claim 51, wherein:
the permissive temperature is below the normal temperature of the ECM; and
the MMP-1 is administered at or below the permissive temperature.

53. The method of claim 52, wherein the MMP-1 is provided in a composition that is at or below the permissive temperature.

54. The method of claim 52, wherein the MMP-1 is mixed with a composition that is at or below the permissive temperature immediately before administration.

55. The method of claim 52, wherein, prior to administration, the ECM is cooled to below the physiological temperature of the body.

56. The method of claim 52, wherein following administration, the ECM is maintained below the physiological temperature of the body for a predetermined time.

57.-61. (canceled)

62. The method of claim 52, wherein the MMP-1 is a zymogen and is processed before administration.

63. The method of claim 62, wherein the MMP-1 is processed by a processing agent.

64. The method of claim 63, wherein the processing agent is selected from among plasmin, plasma kallikrein, trypsin-1, trypsin-2, neutrophil elastase, cathepsin G, tryptase, chymase, proteinase-3, proteinase-3, furin, urinary plasminogen activator (uPA), an active MMP, 4-aminophenylmercuric acetate (AMPA), HgCl2, N-ethylmaleimide, sodium dodecyl sulfate (SDS), chaotropic agents, oxidized glutathione, reactive oxygen, Au(I) salts, acidic pH and heat.

65. The method of claim 64, wherein the active MMP is selected from among an MMP-1, MMP-2, MMP-3, MMP-7, MMP-10, MMP-26 and MT1-MMP.

66. The method of claim 64, wherein the processing agent is AMPA.

67. The method of claim 63, wherein the processing agent is purified away from the modified MMP-1 polypeptide before administration.

68. The method of claim 52, wherein the modified MMP-1 polypeptide is administered at a therapeutically effective amount to treat the disease or condition.

69. The method of claim 52, wherein administration is selected from among subcutaneous, intramuscular, intralesional, intradermal, topical, transdermal, intravenous, oral and rectal.

70. The method of claim 52, wherein administration is sub-epidermal administration.

71. The method of claim 52, wherein administration is subcutaneous administration.

72. The method of claim 52, further comprising administering a pharmacologic agent selected from among other biologics, small molecule compounds, dispersing agents, anesthetics and vasoconstrictors or combinations thereof.

73. The method of claim 72, wherein the dispersing agent is a hyaluronan-degrading enzyme.

74. The method of claim 73, wherein the hyaluronan degrading enzyme is a hyaluronidase.

75. The method of claim 72, wherein the other pharmacologic agent(s) is administered simultaneously, sequentially or intermittently from the MMP-1.

76. The method of claim 72, wherein the other agent(s) is administered prior to administration of the MMP-1.

77. The method of claim 52, wherein the disease or condition of the ECM is a collagen-mediated disease or condition.

78. The method of claim 77, wherein the collagen-mediated disease or condition is selected from among cellulite, Dupuytren's disease, Peyronie's disease, Ledderhose fibrosis, stiff joints, existing scars, scleroderma, lymphedema and coliagenous colitis.

79. The method of claim 78, wherein the collagen-mediated disease or condition is stiff joints that is frozen shoulder.

80. The method of claim 78, wherein the collagen-mediated disease or condition is existing scars that is selected from among surgical adhesions, keloids, hypertrophic scars and depressed scars.

81. The method of claim 52, wherein the ECM-mediated disease or condition is herniated protruding discs.

82. The modified MMP-1 polypeptide of claim 1 that has a sequence of amino acids set forth in any of SEQ ID NOS: 6, 25, 27, 29, 31, 32, 33, 35, 36, 38, 39, 59, 70, 95, 96, 99, 100, 101, 105, 111, 113-115, 125, 132, 148, 160, 181, 182, 185, 195, 209, 218, 219, 232, 233, 235, 238, 248, 253, 254, 261, 262, 264, 284, 301, 305, 317, 324, 341, 354, 365, 384, 388, 397, 420, 429, 436, 440, 460, 467, 476, 483 and 488.
